(19) United States
(12) Patent Application Publication
(10) Pub. No.: US 2026/0232827 A1
(43) Pub. Date: Aug. 13, 2026

(54) ANTIBODY-DRUG CONJUGATES TARGETING NAPI2B AND METHODS OF USE

(71) Applicant: Zymeworks BC Inc., Vancouver (CA)

(72) Inventors: Stuart Daniel Barnscher, Vancouver (CA); Dunja Urosev, Vancouver (CA); James R. Rich, Vancouver (CA); Grant Raymond Wickman, Vancouver (CA); Andrea Hernandez Rojas, Vancouver (CA); Eric ESCOBAR-CABRERA, Burnaby (CA); Robert William William, Vancouver (CA); Samuel Oliver Lawn, Vancouver (CA); Sukhbir Singh Kang, Vancouver (CA); Danny Chui, Vancouver (CA); Brandon Clavette, Vancouver (CA); Duncan Browman, Vancouver (CA); Gesa Volkers, Vancouver (CA)

(21) Appl. No.: 19/644,998

(22) Filed: Apr. 10, 2026

Related U.S. Application Data
(63) Continuation of application No. PCT/CA2024/051381, filed on Oct. 18, 2024.
(60) Provisional application No. 63/544,847, filed on Oct. 19, 2023.
(60) Provisional application No. 63/638,403, filed on Apr. 24, 2024.

(51) Int. Cl. A61K 47/68; A61P 35/00; C07K 16/28

(52) U.S. Cl. CPC A61K 47/68037; A61K 47/6849; A61K 47/6889; A61P 35/00; C07K 16/28

(57) ABSTRACT

Described herein are anti-NaPi2b antibody-drug conjugates (ADCs) comprising an antibody construct that binds human sodium-dependent phosphate transporter 2B (NaPi2b) conjugated to a camptothecin analogue of Formula (I). The antibody construct of the ADC comprises amino acid modifications in the Fc that knock-out binding to Fcγ receptors. ADCs are useful as therapeutics, in particular in the treatment of cancer.

### CROSS-REFERENCE

This application is a continuation of PCT/CA2024/051381, filed Oct. 18, 2024, which claims priority to and the benefit of U.S. Provisional Application No. 63/638,403, filed Apr. 24, 2024, and U.S. Provisional Application No. 63/544,847, filed Oct. 19, 2023, the entire contents of each of which are incorporated by reference herein for all purposes.

### SEQUENCE LISTING

The instant application contains a Sequence Listing which is hereby incorporated by reference in its entirety. Said XML copy, created on Oct. 24, 2024, is named ZYME101WO.xml, and is 91,638 bytes in size.

### FIELD

The present disclosure relates to the field of immunotherapeutics and, in particular, to anti-human sodium-dependent phosphate transporter 2B (hNaPi2b) antibody-drug conjugates.

### BACKGROUND

Sodium-dependent phosphate transporter 2B (NaPi2b) is a transmembrane protein encoded by the SLC34A2 gene. The NaPi2b polypeptide is 690 amino acids in length, with a limited extracellular domain of amino acids 188-361 exposed on the surface of cells. It is widely expressed in normal tissues and overexpressed in a variety of cancers including ovarian cancer, endometrial cancer, and lung cancer.

Given the overexpression of NaPi2b in certain types of cancers, NaPi2b-targeted agents have been studied in clinical trials for the treatment of cancer but have returned mixed results. A Phase I/II clinical trial to study upifitamab rilsodotin, an antibody-drug conjugate (ADC) of the NaPi2b-targeting antibody MX35 with an auristatin-F payload (Dolaflexin platform) in patients with platinum-resistant ovarian cancer or non-small cell lung cancer (NSCLC) was undertaken by Mersana Therapeutics. The NSCLC arm of the study was discontinued due to lack of efficacy, while upifitamab rilsodotin was granted Fast Track Designation for the treatment of platinum-resistant ovarian cancer patients who have received three to four prior lines of therapy. Mersana also completed a Phase I/II clinical trial of XMT-1592 in ovarian cancer; XMT-1592 is a site specific ADC, comprised of antibody MX35 conjugated to an auristatin-F payload using their Dolasynthen platform. The development of this ADC has been discontinued. Lifastuzumab vedotin, an ADC of lifastuzumab with an MMAE payload was studied in a clinical trial sponsored by Genentech in patients with ovarian cancer or NSCLC, but this trial has since been discontinued.

Camptothecin analogues have been developed as payloads for ADCs. Two such ADCs have been approved for treatment of cancer. Trastuzumab deruxtecan (Enhertu™) in which the camptothecin analogue, deruxtecan (Dxd), is conjugated to the anti-HER2 antibody, trastuzumab, via a cleavable tetrapeptide-based linker, and sacituzumab govitecan (Trodelvy™) in which the camptothecin analogue, SN-38, is conjugated to the anti-Trop-2 antibody, sacituzumab, via a hydrolysable, pH-sensitive linker.

Other camptothecin analogues and derivatives, as well as ADCs comprising them have been described. See, for example, International (PCT) Application Publication Nos. WO 2019/195665; WO 2019/236954; WO 2020/200880 and WO 2020/219287.

This background information is provided for the purpose of making known information believed by the applicant to be of possible relevance to the present disclosure available. No admission is necessarily intended, nor should it be construed, that any of the preceding information constitutes prior art against the claimed invention.

### SUMMARY

Described herein are anti-NaPi2b antibody-drug conjugates and methods of use.

A certain aspect of the present disclosure relates to an antibody-drug conjugate having the structure:

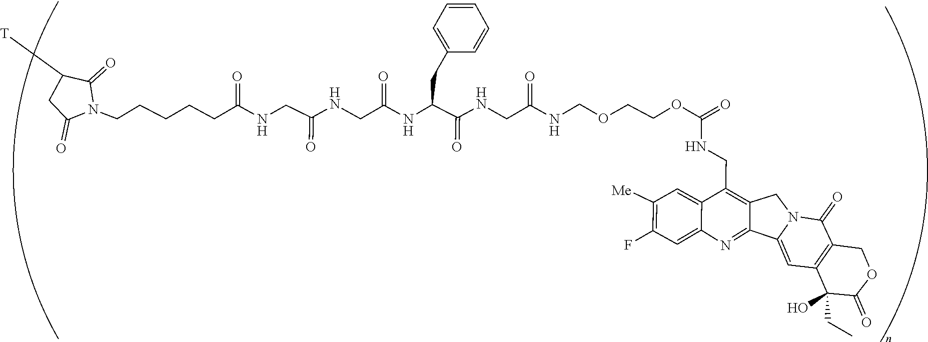

wherein n is between about 2 and about 6, and T is an anti-NaPi2b antibody construct as described herein, wherein T is an anti-NaPi2b antibody construct comprising two heavy chains comprising the sequence as set forth in SEQ ID NO:68, and two light chains comprising the sequence as set forth in SEQ ID NO:67, or wherein T is an anti-NaPi2b antibody construct comprising two heavy chains comprising the sequence as set forth in SEQ ID NO:66, and two light chains comprising the sequence as set forth in SEQ ID NO:67.

Another aspect of the present disclosure relates to a pharmaceutical composition comprising an antibody-drug conjugate as described herein, and a pharmaceutically acceptable carrier or diluent.

Another aspect of the present disclosure relates to a method of inhibiting the proliferation of cancer cells comprising contacting the cells with an effective amount of the antibody-drug conjugate as described herein.

Another aspect of the present disclosure relates to a method of killing cancer cells comprising contacting the cells with an effective amount of the antibody-drug conjugate as described herein.

Another aspect of the present disclosure relates to a method of treating cancer in a subject in need thereof comprising administering to the subject an effective amount of the antibody-drug conjugate as described herein.

Another aspect of the present disclosure relates to an antibody-drug conjugate as described herein for use in therapy.

Another aspect of the present disclosure relates to an antibody-drug conjugate as described herein for use in the treatment of cancer.

Another aspect of the present disclosure relates to a use of an antibody-drug conjugate as described herein in the manufacture of a medicament for the treatment of cancer.

Another aspect of the present disclosure relates to a kit comprising the antibody-drug conjugate as described herein and a label and/or package insert containing instructions for use.

### BRIEF DESCRIPTION OF THE DRAWINGS

FIG. 1A shows the sequence of the mouse heavy chain variable domain CDRs of the chimeric anti-NaPi2b antibody v23855 ported onto a human VH germline (IGHV1-46*03), and FIG. 1B shows the sequence of the mouse light chain variable domain CDRs of chimeric antibody v23855 ported onto a human VL framework (IGKVID-39*01). The CDRs were assigned with the AbM definition and are marked in bold and underlined.

FIG. 2A shows non-reducing (NR) SDS-PAGE profiles for all humanized variants and the parental chimeric variant 23855. FIG. 2B shows reducing (R) SDS-PAGE profiles for all humanized variants and parental chimeric variant 23855. FIG. 2C shows the UPLC-SEC profile for the parental mouse-human chimeric antibody v23855. FIG. 2D shows the UPLC-SEC profile for a representative humanized antibody, v29456.

FIG. 3A depicts binding of humanized antibody variant v29456, MX-35 (v18992) and lifastuzumab (v18993) to human NaPi2b. FIG. 3B depicts binding of humanized antibody variant v29456, MX-35 (v18992) and lifastuzumab (v18993) to cynomolgus NaPi2b. FIG. 3C depicts binding of humanized antibody variant v29456, MX-35 (v18992) and lifastuzumab (v18993) to mouse NaPi2b.

FIG. 4A depicts the N-curve analysis of binding to NaPi2b expressed on IGROV-1 cells for v29814. FIG. 4B depicts the N-curve analysis of binding to NaPi2b expressed on IGROV-1 cells for v36123. FIG. 4C depicts the N-curve analysis of binding to NaPi2b expressed on IGROV- 1 cells for v36124. For each panel, the right curve shows the data for 500 pM constant binding partner and the left curve shows the data for 50 pM constant binding partner.

FIG. 5A shows a comparison of the ability of v23855 (parental chimeric), v29456 (H1L2), v18992 (MX35), and v18993 (lifastuzumab) to internalize in HCC-78 cells. FIG. 5B shows a comparison of the ability of v23855 (parental chimeric), v29456 (H1L2), v18992 (MX35), and v18993 (lifastuzumab) to internalize in NCI-H441 cells.

FIG. 6 depicts the binding of parental chimeric antibody (v23855), humanized antibody variants v29452 and v29456, in addition to binding of MX35 and lifastuzumab ADCs to IGROV-1 cells.

FIG. 7 depicts the ability of v29456 ADCs to exert a bystander effect.

FIG. 8A depicts the cytotoxicity of v29456 ADCs in 2D monolayer cultures of HCC-78 cells. FIG. 8B depicts the cytotoxicity of v29456 ADCs in 2D monolayer cultures of IGROV-1 cells. FIG. 8C depicts the cytotoxicity of v29456 ADCs in 2D monolayer cultures of HCT116 cells.

FIG. 9A depicts the cytotoxicity of v29456 ADCs in 2D monolayer cultures of IGROV-1 cells. FIG. 9B depicts the cytotoxicity of v29456 ADCs in 2D monolayer cultures of TOV-21G cells.

FIG. 10A depicts the cytotoxicity of v29456 ADCs in 3D spheroids of HCC-78 cells. FIG. 10B depicts the cytotoxicity of v29456 ADCs in 3D spheroids of IGROV-1 cells.

FIG. 11A depicts the cytotoxicity of v29456 ADCs in 3D spheroids of IGROV-1 cells. FIG. 11B depicts the cytotoxicity of v29456 ADCs in 3D spheroids of TOV-21G cells.

FIG. 12 depicts the efficacy of v29456 ADCs in an OVCAR3 xenograft model of ovarian cancer.

FIG. 13 depicts the efficacy of v29456 conjugated to DXd1 in an NCI-H441 xenograft model of lung cancer.

FIG. 14A depicts the efficacy of v29456 ADCs in an NCI-H441 xenograft model of lung cancer when dosed at 0.3 mg/kg. FIG. 14B depicts the efficacy of v29456 ADCs in an NCI-H441 xenograft model of lung cancer when dosed at 1 mg/kg.

FIG. 15A depicts the efficacy of v29456 ADCs in a patient-derived (PDX) CTG-2025 model of ovarian cancer. FIG. 15B depicts the efficacy of v29456 ADCs in a patient-derived (PDX) CTG-0958 model of ovarian cancer.

FIG. 16 shows the PK profile of v29456 ADCs in Tg32 mice.

FIG. 17A shows the ability of an ADC of v29456 (H1L2) to internalize in OVCAR-3 cells compared to v18992 (MX35) and v18993 (lifastuzumab). FIG. 17B shows the ability of an ADC of v29456 (H1L2) to internalize in IGROV-1 cells compared to v18992 (MX35) and v18993 (lifastuzumab).

FIG. 18A depicts the cytotoxicity of a v29456 ADC in 3D spheroids of IGROV-1 cells. FIG. 18B depicts the cytotoxicity of a v29456 ADCs in 3D spheroids of NCI-H441 cells. FIG. 18C depicts the cytotoxicity of a v29456 ADCs in 3D spheroids of TOV-21G cells.

FIG. 19A depicts the result of the Membrane Proteome Array™ assay using v38591. FIG. 19B depicts validation data for CLDN3.

FIG. 20 depicts cellular binding of v38591 and v38591 ADCs on IGROV-1 and OVCAR-3 cells.

FIG. 21 depicts the cross-reactivity of v38591 and v38591 ADCs to cynomolgus monkey and mouse NaPi2b.

FIG. 22 shows the specificity v38591 and v38591 ADCs to human NaPi2b, NaPi2a, and NaPi2c.

FIG. 23 shows the internalization of an anti-NaPi2b ADC and naked antibody.

FIG. 24 depicts the bystander activity of anti-NaPi2b ADCs against a NaPi2b-negative EBC-1 cell line.

FIG. 25 shows the effect of DAR4 and DAR8 anti-NaPi2b ADCs on tumor growth in a CTG-0703 xenograft model.

FIG. 26 shows the effect of DAR4 and DAR8 anti-NaPi2b ADCs on tumor growth in a CTG-1301 xenograft model.

FIG. 27 shows the effect of DAR4 and DAR8 anti-NaPi2b ADCs on tumor growth in a CTG-3718 xenograft model.

FIG. 28 shows the effect of DAR4 and DAR8 anti-NaPi2b ADCs on tumor growth in a CTG-1703 xenograft model.

FIG. 29 shows the effect of DAR4 and DAR8 anti-NaPi2b ADCs on tumor growth in a CTG-2025 xenograft model.

FIG. 30 shows the effect of DAR4 and DAR8 anti-NaPi2b ADCs on tumor growth in a CTG-0958 xenograft model.

FIG. 31 depicts pharmacokinetic profiles for DAR4 and DAR8 anti-NaPi2b ADCs in cynomolgus monkeys.

FIG. 32A shows the effect of DAR4 and DAR8 anti-NaPi2b ADCs on tumor growth in a LU5213 lung PDX model. FIG. 32B shows the effect of DAR4 and DAR8 anti-NaPi2b ADCs on tumor growth in a LU5245 lung PDX model. FIG. 32C shows the effect of DAR4 and DAR8 anti-NaPi2b ADCs on tumor growth in a LU6802 lung PDX model. FIG. 32D shows the effect of DAR4 and DAR8 anti-NaPi2b ADCs on tumor growth in a LU6904 lung PDX model. FIG. 32E shows the effect of DAR4 and DAR8 anti-NaPi2b ADCs on tumor growth in a LU11692 lung PDX model. FIG. 32F shows the effect of DAR4 and DAR8 anti-NaPi2b ADCs on tumor growth in a LU11796 lung PDX model. FIG. 32G shows the effect of DAR4 and DAR8 anti-NaPi2b ADCs on tumor growth in a LU11870 lung PDX model. FIG. 32H shows the effect of DAR4 and DAR8 anti-NaPi2b ADCs on tumor growth in a LU11876 lung PDX model.

FIG. 33A shows the effect of DAR4 and DAR8 anti-NaPi2b ADCs on tumor growth in a UT14026 PDX model of endometrial cancer. FIG. 33B shows the effect of DAR4 and DAR8 anti-NaPi2b ADCs on tumor growth in a UT5318 PDX model of endometrial cancer. FIG. 33C shows the effect of DAR4 and DAR8 anti-NaPi2b ADCs on tumor growth in a UT5326 PDX model of endometrial cancer. FIG. 33D shows the effect of DAR4 and DAR8 anti-NaPi2b ADCs on tumor growth in a UT5321 PDX model of endometrial cancer.

FIG. 34 shows the binding of v38591 and v40502 (LALADS) ADCs, parental antibodies, and controls to IGROV-1, HCC-78, H441, and EBC-1 cells.

FIGS. 35A-C depict internalization of v38591 and v40502 (LALADS) antibodies and ADCs in NaPi2b-expressing cells, i.e., IGROV-1 (FIG. 35A), HCC-78 (FIG. 35B) and H441 (FIG. 35C).

FIG. 36 depicts cytotoxicity of v38591 and v40502 (LALADS) antibodies and ADCs in 3D spheroids of NaPi2b-expressing cells.

FIG. 37A depicts the ability of anti-NaPi2b ADCs including v40502 (LALADS)-MC-GGFG-AM-Compound 139 DAR 4 to mediate ADCC. FIG. 37B depicts the ability of anti-NaPi2b ADCs including v40502 (LALADS)-MC-GGFG-AM-Compound 139 DAR 4 to mediate ADCP.

FIG. 38 demonstrates the ability of anti-NaPi2b ADCs including v40502 (LALADS)-MC-GGFG-AM-Compound 139 DAR 4 to inhibit tumor growth in an H441 xenograft model of lung cancer.

FIG. 39 depicts the ability of anti-NaPi2b ADCs including v40502 (LALADS)-MC-GGFG-AM-Compound 139 DAR 4 to inhibit tumor growth in a LU6802 xenograft model of lung cancer.

FIG. 40A depicts the cytotoxic effect of anti-NaPi2b ADCs including v40502 (LALADS)-MC-GGFG-AM-Compound 139 DAR 4 on erythroid progenitor cells. FIG. 40B depicts the cytotoxic effect of anti-NaPi2b ADCs including v40502 (LALADS)-MC-GGFG-AM-Compound 139 DAR 4 on myeloid progenitor cells.

FIG. 41A shows the effects of v38591-MC-GGFG-AM-Compound 139 ADC with a wild-type Fc as well as that of the Fc silenced v40502 (LALADS)-MC-GGFG-AM-Compound 139 ADC on tumor growth in a UT5321 endometrial cancer PDX model in NOD/SCID mice. FIG. 41B shows the total antibody pharmacokinetics of the v38591-MC-GGFG-AM-Compound 139 ADC with a wild-type Fe and that of the Fc silenced v40502 (LALADS)-MC-GGFG-AM-Compound 139 ADC in a UT5321 endometrial cancer PDX model in NOD/SCID mice.

### DETAILED DESCRIPTION

The present disclosure relates to antibody-drug conjugates (ADCs) comprising an antibody construct that binds sodium-dependent phosphate transporter 2B (NaPi2b) (an anti-NaPi2b antibody construct) conjugated to a camptothecin analogue of Formula (I) as described herein. The ADCs of the present disclosure may find use, for example, as therapeutics, in particular in the treatment of cancer.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

Where a range of values is provided herein, for example where a value is defined as being “between” an upper limit value and a lower limit value, it is understood that the range encompasses both the upper limit value and the lower limit value as well as each intervening value.

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

A “complementarity determining region” or “CDR” is an amino acid sequence that contributes to antigen-binding specificity and affinity of an antibody construct described herein. “Framework” regions (FR) can aid in maintaining the proper conformation of the CDRs to promote binding between the antigen-binding region and an antigen. From N-terminus to C-terminus, both the light chain variable region (VL) and the heavy chain variable region (VH) of an antibody typically comprise the domains FR1, CDR1, FR2, CDR2, FR3, CDR3, and FR4. The three heavy chain CDRs are referred to herein as HCDR1, HCDR2, and HCDR3, and the three light chain CDRs are referred to as LCDR1, LCDR2, and LCDR3. CDRs provide the majority of contact residues for the binding of the antibody to the antigen or epitope. Often, the three heavy chain CDRs and the three light chain CDRs are required to bind antigen. However, in some instances, even a single variable domain can confer binding specificity to the antigen. Furthermore, as is known in the art, in some cases, antigen-binding may also occur through a combination of a minimum of one or more CDRs selected from the VH and/or VL domains, for example HCDR3.

A number of different definitions of the CDR sequences are in common use, including those described by Kabat et al. (1983, Sequences of Proteins of Immunological Interest, NIH Publication No. 369-847, Bethesda, MD), by Chothia et al. (1987, J Mol Biol, 196:901-917), as well as the IMGT, AbM (University of Bath) and Contact (MacCallum, et al., 1996, J Mol Biol, 262(5):732-745) definitions. By way of example, CDR definitions according to Kabat, Chothia, IMGT, AbM and Contact are provided in Table 1 below. Accordingly, as would be readily apparent to one skilled in the art, the exact numbering and placement of CDRs may differ based on the numbering system employed. However, it is to be understood that the disclosure herein of a VH includes the disclosure of the associated (inherent) heavy chain CDRs (HCDRs) as defined by any of the known numbering systems. Similarly, disclosure herein of a VL includes the disclosure of the associated (inherent) light chain CDRs (LCDRs) as defined by any of the known numbering systems.

TABLE 1
Common CDR Definitions¹

| | Heavy Chain | | | Light Chain | | |
|---|---|---|---|---|---|---|
| Definition | CDR1² | CDR2 | CDR3 | CDR1 | CDR2 | CDR3 |
| Kabat | H31-H35B | H50-H65 | H95-H102 | L24-L34 | L50-L56 | L89-L97 |
| Chothia | H26-H32, | H52-H56 | H95-H102 | L24-L34 | L50-L56 | L89-L97 |
| | H33 or H34 | | | | | |
| IMGT | H26-H33, | H51-H57 | H93-H102 | L27-L32 | L50-L52 | L89-L97 |
| | H34, H35, | | | | | |
| | H35A or H35B | | | | | |
| AbM | H26-H35B | H50-H58 | H95-H102 | L24-L34 | L50-L56 | L89-L97 |
| Contact | H30-H35B | H47-H58 | H93-H101 | L30-L36 | L46-L55 | L89-L96 |

¹Either the Kabat or Chothia numbering system may be used for HCDR2, HCDR3 and the light chain CDRs for all definitions except Contact, which uses Chothia numbering
²Using Kabat numbering. The position in the Kabat numbering scheme that demarcates the end of the Chothia and IMGT CDR-H1 loop varies depending on the length of the loop because Kabat places insertions outside of those CDR definitions at positions 35A and 35B. However, the IMGT and Chothia CDR-H1 loop can be unambiguously defined using Chothia numbering. CDR-H1 definitions using Chothia numbering: Kabat H31-H35, Chothia H26-H32, AbM H26-H35, IMGT H26-H33, Contact H30-H35.

The term “identical” in the context of two or more polynucleotide or polypeptide sequences, refers to two or more sequences or subsequences that are the same. Sequences are “substantially identical” if they have a percentage of amino acid residues or nucleotides that are the same (for example, about 80%, about 85%, about 90%, about 95%, or about 98% identity, over a specified region) when compared and aligned for maximum correspondence over a comparison window or over a designated region as measured using one of the commonly used sequence comparison algorithms as known to persons of ordinary skill in the art or by manual alignment and visual inspection. For sequence comparison, typically test sequences are compared to a designated reference sequence. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window” refers to a segment of a sequence comprising contiguous amino acid or nucleotide positions which may be, for example, from about 10 to 600 contiguous amino acid or nucleotide positions, or from about 10 to about 200, or from about 10 to about 150 contiguous amino acid or nucleotide positions over which a test sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, for example, by the local homology algorithm of Smith & Waterman, 1970, Adv. Appl. Math., 2:482c; by the homology alignment algorithm of Needleman & Wunsch, 1970, J. Mol. Biol., 48:443; by the search for similarity method of Pearson & Lipman, 1988, Proc. Natl. Acad. Sci. USA, 85:2444, or by computerized implementations of these algorithms (for example, GAP, BESTFIT, FASTA or TFASTA in the Wisconsin Genetics Software Package, Genetics Computer Group, Madison, WI), or by manual alignment and visual inspection (see, for example, Ausubel et al., Current Protocols in Molecular Biology, (1995 supplement), Cold Spring Harbor Laboratory Press). Examples of available algorithms suitable for determining percent sequence identity are the BLAST and BLAST 2.0 algorithms, which are described in Altschul et al., 1997, Nuc. Acids Res., 25:3389-3402, and Altschul et al., 1990, J. Mol. Biol., 215:403-410, respectively. Software for performing BLAST analyses is publicly available through the website for the National Center for Biotechnology Information (NCBI).

The term “acyl,” as used herein, refers to the group —C(O)R, where R is hydrogen, alkyl, aryl, heteroaryl, cycloalkyl or heterocycloalkyl.

The term “acyloxy” refers to the group —OC(O)R, where R is alkyl.

The term “alkoxy,” as used herein, refers to the group —OR, where R is alkyl, aryl, heteroaryl, cycloalkyl or cycloheteroalkyl.

The term “alkyl,” as used herein, refers to a straight chain or branched saturated hydrocarbon group containing the specified number of carbon atoms. Examples of alkyl include, but are not limited to, methyl, ethyl, n-propyl, isopropyl, n-butyl, sec-butyl, isobutyl, t-butyl, pentyl, isopentyl, t-pentyl, neo-pentyl, 1-methylbutyl, 2-methylbutyl, n-hexyl, and the like.

The term “alkylaminoaryl,” as used herein, refers to an alkyl group as defined herein substituted with one aminoaryl group as defined herein.

The term “alkylheterocycloalkyl,” as used herein, refers to an alkyl group as defined herein substituted with one heterocycloalkyl group as defined herein.

The term “alkylthio,” as used herein, refers to the group —SR, where R is an alkyl group.

The term “amido,” as used herein, refers to the group —C(O)NRR′, where R and R′ are independently hydrogen, alkyl, aryl, heteroaryl, cycloalkyl or heterocycloalkyl.

The term “amino,” as used herein, refers to the group —NRR′, where R and R′ are independently hydrogen, alkyl, aryl, heteroaryl, cycloalkyl or heterocycloalkyl.

The term “aminoalkyl,” as used herein, refers to an alkyl group as defined herein substituted with one or more amino groups, for example, one, two or three amino groups.

The term “aminoaryl,” as used herein, refers to an aryl group as defined herein substituted with one amino group.

The term “aryl,” as used herein, refers to a 6- to 12-membered mono- or bicyclic hydrocarbon ring system in which at least one ring aromatic. Examples of aryl include, but are not limited to, phenyl, naphthalenyl, 1,2,3,4-tetrahydro-naphthalenyl, 5,6,7,8-tetrahydro-naphthalenyl, indanyl, and the like.

The term “carboxy,” as used herein, refers to the group —C(O)OR, where R is H, alkyl, aryl, heteroaryl, cycloalkyl or cycloheteroalkyl.

The term “cyano,” as used herein, refers to the group —CN.

The term “cycloalkyl,” as used herein, refers to a mono- or bicyclic saturated hydrocarbon containing the specified number of carbon atoms. Examples of cycloalkyl include, but are not limited to, cyclopropyl, cyclobutyl, cyclopentyl, cyclohexyl, cycloheptane, bicyclo [2.2.1]heptane, bicyclo [3.1.1]heptane, and the like.

The term “haloalkyl,” as used herein, refers to an alkyl group as defined herein substituted with one or more halogen atoms.

The terms “halogen” and “halo,” as used herein refer to fluorine (F), bromine (Br), chlorine (Cl) and iodine (I).

The term “heteroaryl,” as used herein, refers to a 6- to 12-membered mono- or bicyclic ring system in which at least one ring atom is a heteroatom and at least one ring is aromatic. Examples of heteroatoms include, but are not limited to, O, S and N. Examples of heteroaryl include, but are not limited to: pyridyl, benzofuranyl, pyrazinyl, pyridazinyl, pyrimidinyl, triazinyl, quinolinyl, benzoxazolyl, benzothiazolyl, isoquinolinyl, quinazolinyl, quinoxalinyl, pyrrolyl, indolyl, and the like.

The term “heterocycloalkyl,” as used herein, refers to a mono- or bicyclic non-aromatic ring system containing the specified number of atoms and in which at least one ring atom is a heteroatom, for example, O, S or N. A heterocyclyl substituent can be attached via any of its available ring atoms, for example, a ring carbon, or a ring nitrogen. Examples of heterocycloalkyl include, but are not limited to, aziridinyl, azetidinyl, piperidinyl, morpholinyl, piperazinyl, pyrrolidinyl, and the like.

The terms “hydroxy” and “hydroxyl,” as used herein, refer to the group —OH.

The term “hydroxyalkyl,” as used herein, refers to an alkyl group as defined herein substituted with one or more hydroxy groups.

The term “nitro,” as used herein, refers to the group —NO₂.

The term “sulfonyl,” as used herein, refers to the group —S(O)₂R, where R is H, alkyl or aryl.

The term “sulfonamido,” as used herein, refers to the group —NH—S(O)₂R, where R is H, alkyl or aryl.

The terms “thio” and “thiol,” as used herein, refer to the group —SH.

Unless specifically stated as being “unsubstituted,” any alkyl, cycloalkyl, heterocycloalkyl, aryl or heteroaryl group referred to herein is understood to be “optionally substituted,” i.e. each such reference includes both unsubstituted and substituted versions of these groups. For example, reference to a “—C1-C6 alkyl” includes both unsubstituted —C1-C6 alkyl and —C1-C6 alkyl substituted with one or more substituents. Examples of substituents include, but are not limited to, halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl or heteroaryl. In certain embodiments, each alkyl, cycloalkyl, heterocycloalkyl, aryl or heteroaryl group referred to herein is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

A chemical group described herein as “substituted,” may include one substituent or a plurality of substituents up to the full valence of substitution for that group. For example, a methyl group may include 1, 2, or 3 substituents, and a phenyl group may include 1, 2, 3, 4, or 5 substituents. When a group is substituted with more than one substituent, the substituents may be the same or they may be different.

The term “subject,” as used herein, refers to an animal, in some embodiments a mammal, which is the object of treatment, observation or experiment. The animal may be a human, a non-human primate, a companion animal (for example, dog, cat, or the like), farm animal (for example, cow, sheep, pig, horse, or the like) or a laboratory animal (for example, rat, mouse, guinea pig, non-human primate, or the like). In certain embodiments, the subject is a human.

It is contemplated that any embodiment discussed herein can be implemented with respect to any method, use or composition disclosed herein, and vice versa.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Antibody-Drug Conjugates

The present disclosure relates to antibody-drug conjugates (ADCs) comprising an anti-NaPi2b antibody construct conjugated to a camptothecin analogue having Formula (I). In certain embodiments, the ADC has Formula (X):

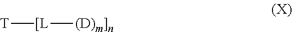

wherein:
T is an anti-NaPi2b antibody construct as described herein;
L is a linker;
D is a camptothecin analogue as described herein;
m is an integer between 1 and 4, and
n is an integer between 1 and 10.

Components of Formula (X) are described below.

In one embodiment, T is an anti-NaPi2b antibody construct having a silenced Fc region. ADCs comprising such Fc-silenced anti-NaPi2b antibodies are a subset of the ADCs of the present disclosure and are referred to herein as anti-NaPi2b Fc-silenced ADCs. Additional description of anti-NaPi2b Fc-silenced ADCs is found in Examples 39 to 58. In some embodiments, an anti-NaPi2b Fc-silenced ADC of the present disclosure comprises LALADS mutations in its Fc region, as further described herein.

### Anti-NaPi2b Antibody Constructs

The ADCs of the present disclosure comprise an anti-NaPi2b antibody construct. In this context, the term “antibody construct” refers to a polypeptide or a set of polypeptides that comprises one or more antigen-binding domains, where each of the one or more antigen-binding domains specifically binds to an epitope or antigen. Where the antibody construct comprises two or more antigen-binding domains, each of the antigen-binding domains may bind to the same epitope or antigen (i.e. the antibody construct is monospecific) or they may bind to different epitopes or antigens (i.e. the antibody construct is bispecific or multispecific). The antibody construct may further comprise a scaffold and the one or more antigen-binding domains can be fused or covalently attached to the scaffold, optionally via a linker. In some embodiments, the scaffold is an Fc region, e.g., a human IgG Fc region.

In accordance with the present disclosure, the anti-NaPi2b antibody construct of the ADC comprises at least one antigen-binding domain that specifically binds to human NaPi2b (hNaPi2b). By “specifically binds” to hNaPi2b, it is meant that the antibody construct binds to hNaPi2b but does not exhibit significant binding to NaPi2a or NaPi2c. In certain embodiments, the anti-NaPi2b antibody constructs of the present disclosure may be capable of binding to an NaPi2b from one or more non-human species. In certain embodiments, the anti-NaPi2b antibody constructs of the present disclosure are capable of binding to cynomolgus monkey NaPi2b.

Human NaPi2b is also known as human “solute carrier family 34 member 2” or “SLC34A2.” The protein sequences of hNaPi2b from various sources are known in the art and readily available from publicly accessible databases, such as GenBank or UniProtKB. Examples of hNaPi2b sequences include for example those provided under NCBI reference numbers NP_006415.3, NP_001171470.2, and NP_001171469.2. An exemplary hNaPi2b protein sequence is provided in Table 2 as SEQ ID NO:1 (UniProt ID: 095436). An exemplary cynomolgus monkey NaPi2b protein sequence is also provided in Table 2 (SEQ ID NO:2; UniProt ID: A0A2K5UHY1), as is an exemplary mouse NaPi2b protein sequence (SEQ ID NO:3; UniProt ID: Q9DBP0).

TABLE 2
Human, Cynomolgus Monkey and Mouse NaPi2b Protein Sequences

| | | SEQ |
|---|---|---|
| Organism | Sequence | ID NO |
| Human | MAPWPELGDAQPNPDKYLEGAAGQQPTAPDKSKETNKTDNTEAPV | 1 |
| | TKIELLPSYSTATLIDEPTEVDDPWNLPTLQDSGIKWSERDTKGKILC | |
| | FFQGIGRLILLLGFLYFFVCSLDILSSAFQLVGGKMAGQFFSNSSIMS | |
| | NPLLGLVIGVLVTVLVQSSSTSTSIVVSMVSSSLLTVRAAIPIIMGANI | |
| | GTSITNTIVALMQVGDRSEFRRAFAGATVHDFFNWLSVLVLLPVEV | |
| | ATHYLEIITQLIVESFHFKNGEDAPDLLKVITKPFTKLIVQLDKKVISQ | |
| | IAMNDEKAKNKSLVKIWCKTFTNKTQINVTVPSTANCTSPSLCWTD | |
| | GIQNWTMKNVTYKENIAKCQHIFVNFHLPDLAVGTILLILSLLVLCG | |
| | CLIMIVKILGSVLKGQVATVIKKTINTDFPFPFAWLTGYLAILVGAG | |
| | MTFIVQSSSVFTSALTPLIGIGVITIERAYPLTLGSNIGTTTTAILAALA | |
| | SPGNALRSSLQIALCHFFFNISGILLWYPIPFTRLPIRMAKGLGNISAK | |
| | YRWFAVFYLIIFFFLIPLTVFGLSLAGWRVLVGVGVPVVFIIILVLCLR | |
| | LLQSRCPRVLPKKLQNWNFLPLWMRSLKPWDAVVSKFTGCFQMR | |
| | CCCCCRVCCRACCLLCDCPKCCRCSKCCEDLEEAQEGQDVPVKAP | |
| | ETFDNITISREAQGEVPASDSKTECTAL | |
| Cynomolgus | MAPWPELGDAQPNPSKYLEGATSQQPITPDKSKETNKTDNTEVPVT | 2 |
| Monkey | KFELLPTYSTATLIEEPTEVDDPWNLPTLQDSGIKWSERDAKGKILC | |
| | VFQGIGRLILLLGFLYFFVCSLDVLSSAFQLVGGKMAGQFFSNSSIMS | |
| | NPLLGLVIGVLVTVFVQSSSTSTSIVVSMVASSLLTVRAAIPIIMGANI | |
| | GTSITNTIVALMQVGDRSEFRRAFAGATVHDFFNWLSVLVLLPVEV | |
| | ATHYLEVVTQLIVESFHFKNGEDAPDLLKVITKPFTKLIVQLDKKVIS | |
| | QIAMNDETAKNKSLVKIWCKTFTNMTQMNVTVPSMANCTSHSLC | |
| | WTDGIQIWTMKNVTYKENIAKCQHIFVNFHLPDLAVGIILLIISLLVL | |
| | CGCLIMIVKILGSVLKGQVATVIKKTINTDFPFPFAWLTGYLAILVGA | |
| | GMTFIVQSSSVFTSALTPLIGIGVITIERAYPLTLGSNIGTTTTAILAAL | |
| | ASPGNTLRSSLQIALCHFFFNISGILLWYPIPFTRLPIRMAKGLGNISA | |
| | KYRWFAVFYLIIFFFLIPLTVFGLSLAGWPVLVGVGVPVVFIIILVLCL | |
| | RLLQSRCPRVLPKKLQNWNFLPLCMHSLKPWDAVISKFTGPFQMPC | |
| | CCCCRVCCRVCCLLCGCPKCCRCSKCCQDLEEGQEAQGVPVKAPE | |
| | TFDNITISREAQGEVRAPDSKTECTAL | |
| Mouse | MAPWPELENAQPNPGKFIEGASGPQSSIPAKDKEASKTNDNGTPVA | 3 |
| | KTELLPSYSALVLIEEHPEGTDPWDLPELQDTGIKWSERDTKGKTLC | |
| | IFQGVGKFILLLGFLYLFVCSLDVLSSAFQLVGGKVAGQFFSNNSIM | |
| | SNPVAGLVIGVLVTVMVQSSSTSSSIIVSMVASSLLTVRAAIPIIMGA | |
| | NIGTSITNTIVALMQAGDRNEFRRAFAGATVHDFFNWLSVFVLLPLE | |
| | AATHYLEILTNLVLETFKFQNGEDAPDILKVITDPFTKLIIQLDKKVI | |
| | QQIAMGDSAAQNKSLIKIWCKSITNVTEMNVTVPSTDNCTSPSYCW | |
| | TDGIQTWTIQNVTQKENIAKCQHIFVNFSLPDLAVGIILLTVSLVVLC | |
| | GCLIMIVKLLGSVLRGQVATVIKKTLNTDFPFPFAWLTGYLAILVGA | |
| | GMTFIVQSSSVFTSAMTPLIGIGVISIERAYPLTLGSNIGTTTTAILAAL | |
| | ASPGNTLRSSLQIALCHFFFNISGILLWYPIPFTRLPIRLAKGLGNISAK | |
| | YRWFAVFYLIFFFFVTPLTVFGLSLAGWPVLVGVGVPIILLLLLVLCL | |
| | RMLQFRCPRILPLKLRDWNFLPLWMHSLKPWDNVISLATTCFQRRC | |
| | CCCCRVCCRVCCMVCGCKCCRCSKCCRDQGEEEEEKEQDIPVKAS | |
| | GAFDNAAMSKECQDEGKGQVEVLSMKALSNTTVF | |

Specific binding of an antigen-binding domain to a target antigen or epitope may be measured, for example, through an enzyme-linked immunosorbent assay (ELISA), a surface plasmon resonance (SPR) technique (employing, for example, a BIAcore instrument) (Liljeblad et al., 2000, Glyco J, 17:323-329), flow cytometry or a traditional binding assay (Heeley, 2002, Endocr Res, 28:217-229). In certain embodiments, specific binding may be defined as the extent of binding to a non-target protein (such as hNaPi2a or hNaPi2c) being less than about 5% to less than about 10% of the binding to hNaPi2b as measured by ELISA or flow cytometry, for example.

The term “dissociation constant (K_{D} or K_{d})” as used herein, is intended to refer to the equilibrium dissociation constant of a particular ligand-protein interaction. As used herein, ligand-protein interactions refer to, but are not limited to protein-protein interactions or antibody-antigen interactions. The K_{D} measures the propensity of two proteins complexed together (e.g. AB) to dissociate reversibly into constituent components (A+B), and is defined as the ratio of the rate constant of dissociation, also called the “off-rate (k_{off})”, to the association rate constant, or “on-rate (kₒₙ)”. Thus, K_{D} equals k_{off}/kₒₙ and is expressed as a molar concentration (M). It follows that the smaller the K_{D}, the stronger the affinity of binding, and thus a decrease in K_{D} indicates an increase in affinity. Therefore, a K_{D} of 1 mM indicates weak binding affinity compared to a K_{D} of 1 nM. Affinity is sometimes measured in terms of a K_{A} or Kₐ, which is the reciprocal of the K_{D} or K_{d}. K_{D} between antibody and its antigen can be determined using methods well established in the art. One method for determining such K_{D} is by using surface plasmon resonance (SPR), typically using a biosensor system such as a Biacore® system. Isothermal titration calorimetry (ITC) is another method that can be used to measure K_{D}. The Octet™ system may also be used to measure the affinity of antibodies for a target antigen.

In certain embodiments, specific binding of an antibody construct for NaPi2b may be defined by a dissociation constant (K_{d} or K_{D}) of ≤1 μM, for example, ≤500 nM, ≤250 nM, ≤100 nM, ≤50 nM, or ≤10 nM. In certain embodiments, specific binding of an antibody construct for a particular antigen or an epitope may be defined by a dissociation constant (K_{D}) of 10⁻⁶ M or less, for example, 10⁻⁷ M or less, or 10⁻⁸ M or less. In some embodiments, specific binding of an antibody construct for a particular antigen or an epitope may be defined by a dissociation constant (K_{D}) between 10⁶ M and 10⁻⁹ M, for example, between 10⁻⁷ M and 10⁻⁹ M. As is known in the art the numerical value of the dissociation constant obtained may vary depending on how it is tested. For example, the expression level of NaPi2b in the cell line, format of the antibody construct (i.e. monovalent or bivalent), and type of assay (i.e. ELISA or flow cytometry), may affect the numerical value of the dissociation constant when measured in a cell-based assay. The data provided in the Examples illustrate this general point, as shown in Examples 10, 11, and 16.

In some embodiments, the anti-NaPi2b antibody constructs of the present disclosure have a K_{d} that is lower than that of reference antibody lifastuzumab, and comparable to that of reference antibody MX35, when measured by flow cytometry in cells that express NaPi2b at high levels. Accordingly, in these embodiments, the anti-NaPi2b antibody constructs of the present disclosure comprise an antigen-binding domain having an affinity for human NaPi2b that is greater than that of reference antibody lifastuzumab and comparable to that of reference antibody MX35.

In certain embodiments, the anti-NaPi2b antibody constructs exhibit comparable levels of internalization to the reference antibody MX35 and exhibit greater levels of internalization compared to reference antibody lifastuzumab in high and mid NaPi2b-expressing cells. In some embodiments, internalization is measured after 4 hours, after 5 hours or after 24 hours of treatment.

Antibody internalization may be measured using art-known methods, for example, by a direct internalization method according to the protocol detailed in Schmidt, M. et al., 2008, Cancer Immunol. Immunother., 57:1879-1890, or using commercially available fluorescent dyes such as the pHAb Dyes (Promega Corporation, Madison, WI), pHrodo iFL and Deep Red Dyes (ThermoFisher Scientific Corporation, Waltham, MA) and Incucyte® Fabfluor-pH Antibody Labeling Reagent (Sartorius AG, Gottingen, Germany) and analysis techniques such as microscopy, FACS, high content imaging or other plate-based assays.

NaPi2b expression varies depending on cell type as indicated throughout the disclosure and the level of NaPi2b expression can be referred to herein as “high”, “mid,” “low” or “negative.” These terms are used for reference to describe levels of expression in general according to the designations shown in Table 15.1 in Example 15 and are not intended to be limited to the specific numerical values for average NaPi2b protein per cell included therein. Alternatively, expression level of NaPi2b in cells or tumors may be assessed by immunohistochemistry (IHC) according to methods known in the art. For example, IHC may be used to stain for NaPi2b in tumor tissue samples from xenograft models, cell-derived (CDX) or patient-derived (PDX). Tissue samples may be examined, and an H-score calculated as known in the art and described, for example in Example 35, herein. The higher the H-score, the higher the expression of NaPi2b in the tissue sample.

### Antigen-Binding Domains

The anti-NaPi2b antibody constructs of ADCs of the present disclosure comprise at least one antigen-binding domain that is capable of binding to hNaPi2b. The at least one antigen-binding domain capable of binding to hNaPi2b typically is an immunoglobulin-based binding domain, such as an antigen-binding antibody fragment. Examples of an antigen-binding antibody fragment include, but are not limited to, a Fab fragment, a Fab′ fragment, a single chain Fab (scFab), a single chain Fv (scFv) and a single domain antibody (sdAb).

A “Fab fragment” contains the constant domain of the light chain (CL) and the first constant domain of the heavy chain (CH1) along with the variable domains of the light and heavy chains (VL and VH, respectively). Fab′ fragments differ from Fab fragments by the addition of a few amino acid residues at the C-terminus of the heavy chain CHI domain, including one or more cysteines from the antibody hinge region. A Fab fragment may also be a single-chain Fab molecule, i.e. a Fab molecule in which the Fab light chain and the Fab heavy chain are connected by a peptide linker to form a single peptide chain. For example, the C-terminus of the Fab light chain may be connected to the N-terminus of the Fab heavy chain in the single-chain Fab molecule.

An “scFv” includes a heavy chain variable domain (VH) and alight chain variable domain (VL) of an antibody in a single polypeptide chain. The scFv may optionally further comprise a polypeptide linker between the VH and VL domains which enables the scFv to form a desired structure for antigen binding. For example, an scFv may include a VL connected from its C-terminus to the N-terminus of a VH by a polypeptide linker. Alternately, an scFv may comprise a VH connected through its C-terminus to the N-terminus of a VL by a polypeptide linker (see review in Pluckthun in The Pharmacology of Monoclonal Antibodies, vol. 113, Rosenburg and Moore eds., Springer-Verlag, New York, pp. 269-315 (1994)).

An “sdAb” format refers to a single immunoglobulin domain. The sdAb may be, for example, of camelid origin. Camelid antibodies lack light chains and their antigen-binding sites consist of a single domain, termed a “VHH.” An sdAb comprises three CDR/hypervariable loops that form the antigen-binding site: CDR1, CDR2 and CDR3. sdAbs are fairly stable and easy to express, for example, as a fusion with the Fc chain of an antibody (see, for example, Harmsen & De Haard, 2007, Appl. Microbiol Biotechnol., 77(1):13-22).

In those embodiments in which the anti-NaPi2b antibody constructs comprise two or more antigen-binding domains, each additional antigen-binding domain may independently be an immunoglobulin-based domain, such as an antigen-binding antibody fragment, or a non-immunoglobulin-based domain, such as a non-immunoglobulin-based antibody mimetic, or other polypeptide or small molecule capable of specifically binding to its target, for example, a natural or engineered ligand. Non-immunoglobulin-based antibody mimetic formats include, for example, anticalins, fynomers, affimers, alphabodies, DARPins and avimers.

The present disclosure describes herein the identification of a mouse antibody that specifically binds hNaPi2b; a mouse-human chimeric variant of this antibody is identified as variant 23855. The anti-NaPi2b antibody construct of the ADC of the present disclosure comprises an antigen-binding domain derived from this mouse antibody or humanized antibody variants of same. Representative humanized antibody variants (e.g., v29449, v29450, v29451, v29452, v29453, v29454, v29455, v29456, v29457, v29458, v29459, and v29460) of the mouse antibody are also described. In certain embodiments, the anti-NaPi2b antibody constructs described herein specifically bind human NaPi2b having the sequence as set forth in SEQ ID NO:1.

In certain embodiments, the anti-NaPi2b antibody construct of the ADC competes with any one of humanized antibody variants v29449, v29450, v29451, v29452, v29453, v29454, v29455, v29456, v29457, v29458, v29459, and v29460, or with parental chimeric antibody v23855, for binding to human NaPi2b. In assessing competition as described below, each of variants v29449, v29450, v29451, v29452, v29453, v29454, v29455, v29456, v29457, v29458, v29459, v29460, and v23855, are referred to as a competition reference antibody.

One skilled in the art can determine whether antibody constructs compete with variants v29449, v29450, v29451, v29452, v29453, v29454, v29455, v29456, v29457, v29458, v29459, and v29460, or parental chimeric antibody v23855 for binding to hNaPi2b using competition assays known in the art. For example, the competition reference antibody is first allowed to bind to hNaPi2b under saturating conditions and then the ability of the test antibody construct to bind to hNaPi2b is measured. If the test antibody construct is able to bind to hNaPi2b at the same time as the competition reference antibody, then the test antibody construct is considered to bind to a different epitope than the competition reference antibody. Conversely, if the test antibody construct is not able to bind to hNaPi2b at the same time as the competition reference antibody, then the test antibody construct is considered to bind to the same epitope, to an overlapping epitope, or to an epitope that is in close proximity to the epitope bound by the competition reference antibody. Such competition assays can be performed using techniques such as ELISA, radioimmunoassay, surface plasmon resonance (SPR), bio-layer interferometry, flow cytometry and the like. An “antibody that competes with” a competition reference antibody refers to an antibody that blocks binding of the reference antibody to its epitope in a competition assay by 50% or more.

In certain embodiments, the anti-NaPi2b antibody constructs of the present disclosure comprise at least one antigen-binding domain that specifically binds to hNaPi2b, where the antigen-binding domain comprises a set of CDRs based on the CDRs of parental chimeric antibody v23855 described herein. The CDR sequences of the parental chimeric antibody v23855 and representative humanized antibody variants are shown in. Table 3.

TABLE 3
CDR sequences of anti-NaPi2b antibody constructs

| Numbering | | | | | | |
|---|---|---|---|---|---|---|
| System | Sequence | | | | | |
| | | SEQ | | SEQ | | SEQ |
| | Heavy Chain | ID | Heavy Chain | ID | Heavy Chain | ID |
| | CDR1 | NO | CDR2 | NO | CDR3 | NO |
| IMGT | GFTFTSYN | 4 | ISPGNGDL | 5 | ARGRTGMGAVDY | 6 |
| Kabat | SYNVH | 7 | AISPGNGDLS | 8 | GRTGMGAVDY | 9 |
| | | | YNQRFKD | | | |
| Chothia | GFTFTSY | 10 | SPGNGD | 11 | GRTGMGAVDY | 9 |
| AbM | GFTFTSYNVH | 12 | AISPGNGDLS | 13 | GRTGMGAVDY | 9 |
| Contact | TSYNVH | 14 | WIAAISPGNG | 15 | ARGRTGMGAVD | 16 |
| | | | DLS | | | |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QDVYSY | 17 | YTS | — | QQYNIFPWT | 18 |
| Kabat | RASQDVYSYLN | 19 | YTSRLQS | 20 | QQYNIFPWT | 18 |
| Chothia | RASQDVYSYLN | 19 | YTSRLQS | 20 | QQYNIFPWT | 18 |
| AbM | RASQDVYSYLN | 19 | YTSRLQS | 20 | QQYNIFPWT | 18 |
| Contact | YSYLNWY | 21 | LLIYYTSRLQ | 22 | QQYNIFPW | 23 |

In certain embodiments, the anti-NaPi2b antibody constructs of the ADC of the present disclosure comprise an antigen-binding domain having heavy chain CDR amino acid sequences (HCDR1, HCDR2 and HCDR3) comprising the sequences as set forth in SEQ ID NOs: 7, 8, and 9, and light chain CDR amino acid sequences (LCDR1, LCDR2 and LCDR3) comprising the sequences as set forth in SEQ ID NOs: 19, 20, and 18.

In certain embodiments, the anti-NaPi2b antibody constructs of the ADC of the present disclosure comprise an antigen-binding domain having heavy chain CDR amino acid sequences (HCDR1, HCDR2 and HCDR3) comprising the sequences as set forth in SEQ ID NOs: 4, 5, and 6, and light chain CDR amino acid sequences (LCDR1 and LCDR3) comprising the sequences as set forth in SEQ ID NO: 17 and SEQ ID NO:18 and the LCDR2 sequence YTS.

In certain embodiments, the anti-NaPi2b antibody constructs of the ADC of the present disclosure comprise an antigen-binding domain having heavy chain CDR amino acid sequences (HCDR1, HCDR2 and HCDR3) comprising the sequences as set forth in SEQ ID NOs: 10, 11, and 9, and light chain CDR amino acid sequences (LCDR1, LCDR2 and LCDR3) comprising the sequences as set forth in SEQ ID NOs: 19, 20, and 18.

In certain embodiments, the anti-NaPi2b antibody constructs of the ADC of the present disclosure comprise an antigen-binding domain having heavy chain CDR amino acid sequences (HCDR1, HCDR2 and HCDR3) comprising the sequences as set forth in SEQ ID NOs: 12, 13, and 9, and light chain CDR amino acid sequences (LCDR1, LCDR2 and LCDR3) comprising the sequences as set forth in SEQ ID NOs: 19, 20, and 18.

In certain embodiments, the anti-NaPi2b antibody constructs of the ADC of the present disclosure comprise an antigen-binding domain having heavy chain CDR amino acid sequences (HCDR1, HCDR2 and HCDR3) comprising the sequences as set forth in SEQ ID NOs: 14, 15, and 16, and light chain CDR amino acid sequences (LCDR1, LCDR2 and LCDR3) comprising the sequences as set forth in SEQ ID NOs: 21, 22, and 23.

In certain embodiments, the anti-NaPi2b antibody constructs of the ADC of the present disclosure comprise an antigen-binding domain having:
(i) an HCDR1 amino acid sequence selected from the HCDR1 amino acid sequences of any one of variants v23855, v29449, v29450, v29451, v29452, v29453, v29454, v29455, v29456, v29457, v29458, v29459, or v29460; an HCDR2 amino acid sequence selected from the HCDR2 amino acid sequences of any one of variants v23855, v29449, v29450, v29451, v29452, v29453, v29454, v29455, v29456, v29457, v29458, v29459, or v29460, and an HCDR3 amino acid sequence selected from the HCDR3 amino acid sequences of any one of variants v23855, v29449, v29450, v29451, v29452, v29453, v29454, v29455, v29456, v29457, v29458, v29459, or v29460, and
(ii) an LCDR1 amino acid sequence selected from the LCDR1 amino acid sequences of any one of variants v23855, v29449, v29450, v29451, v29452, v29453, v29454, v29455, v29456, v29457, v29458, v29459, or v29460; an LCDR2 amino acid sequence selected from the LCDR2 amino acid sequences of any one of variants v23855, v29449, v29450, v29451, v29452, v29453, v29454, v29455, v29456, v29457, v29458, v29459, or v29460, and an LCDR3 amino acid sequence selected from the LCDR3 amino acid sequences of any one of variants v23855, v29449, v29450, v29451, v29452, v29453, v29454, v29455, v29456, v29457, v29458, v29459, or v29460,
where the CDR amino acid sequences are as defined by any one of the IMGT, Chothia, Kabat, Contact or AbM numbering systems (see, e.g., Table 3).

In certain embodiments, the anti-NaPi2b antibody constructs of the ADCs of the present disclosure comprise an antigen-binding domain having heavy chain CDR amino acid sequences (HCDR1, HCDR2 and HCDR3) selected from the heavy chain CDR amino acid sequences of any one of variants v23855, v29449, v29450, v29451, v29452, v29453, v29454, v29455, v29456, v29457, v29458, v29459, or v29460, as defined by any one of the IMGT, Chothia, Kabat, Contact or AbM numbering systems, and light chain CDR amino acid sequences (LCDR1, LCDR2 and LCDR3) selected from the light chain CDR amino acid sequences of any one of variants v23855, v29449, v29450, v29451, v29452, v29453, v29454, v29455, v29456, v29457, v29458, v29459, or v29460, as defined by any one of the IMGT, Chothia, Kabat, Contact or AbM numbering systems.

In certain embodiments, the anti-NaPi2b antibody constructs of ADCs of the present disclosure comprise an antigen-binding domain comprising heavy chain CDR amino acid sequences (HCDR1, HCDR2 and HCDR3) and light chain CDR amino acid sequences (LCDR1, LCDR2 and LCDR3) of any one of variants v23855, v29449, v29450, v29451, v29452, v29453, v29454, v29455, v29456, v29457, v29458, v29459, or v29460, as defined by any one of the IMGT, Chothia, Kabat, Contact or AbM numbering systems.

In certain embodiments, the anti-NaPi2b antibody constructs of ADCs of the present disclosure comprise an antigen-binding domain having a VH sequence comprising the CDR sequences of the VH sequence of any one of variants v23855, v29449, v29450, v29451, v29452, v29453, v29454, v29455, v29456, v29457, v29458, v29459, or v29460. In certain embodiments, the anti-NaPi2b antibody constructs of the present disclosure comprise an antigen-binding domain having a VL sequence comprising the CDR sequences of the VL sequence of any one of variants v23855, v29449, v29450, v29451, v29452, v29453, v29454, v29455, v29456, v29457, v29458, v29459, or v29460.

One skilled in the art will appreciate that a limited number of amino acid substitutions may be introduced into the CDR sequences or into the VH or VL sequences of known antibodies without the antibody losing its ability to bind its target. Candidate amino acid substitutions may be identified by computer modeling or by art-known techniques such as alanine scanning, with the resulting variants being tested for binding activity by standard techniques. Accordingly, in certain embodiments, the anti-NaPi2b antibody constructs of the present disclosure comprise an antigen-binding domain that comprises a set of CDRs (i.e. heavy chain HCDR1, HCDR2 and HCDR3, and light chain LCDR1, LCDR2 and LCDR3) that have 90% or greater, 95% or greater, 98% or greater, 99% or greater, or 100% sequence identity to a set of CDRs of any one of variants v23855, v29449, v29450, v29451, v29452, v29453, v29454, v29455, v29456, v29457, v29458, v29459, or v29460, where the % sequence identity is calculated across all six CDRs and where the antigen-binding domain retains the ability to bind hNaPi2b.

In certain embodiments, the anti-NaPi2b antibody constructs of the present disclosure comprise an antigen-binding domain that comprises a variant of the set of CDR sequences of any one of variants v23855, v29449, v29450, v29451, v29452, v29453, v29454, v29455, v29456, v29457, v29458, v29459, or v29460, where the variant comprises between 1 and 10 amino acid substitutions across the set of CDRs (i.e. the CDRs may be modified by up to 10 amino acid substitutions with any combination of the six CDRs being modified), and where the antigen-binding domain retains the ability to bind hNaPi2b. In some embodiments, the anti-NaPi2b antibody constructs of the present disclosure comprise an antigen-binding domain that comprises a variant of the set of CDR sequences of any one of variants v23855, v29449, v29450, v29451, v29452, v29453, v29454, v29455, v29456, v29457, v29458, v29459, or v29460, where the variant comprises between 1 and 7 amino acid substitutions, between 1 and 5 amino acid substitutions, between 1 and 4 amino acid substitutions, between 1 and 3 amino acid substitutions, between 1 and 2 amino acid substitutions, or 1 amino acid substitution, across the set of CDRs, and where the antigen-binding domain retains the ability to bind hNaPi2b.

In certain embodiments, the anti-NaPi2b antibody constructs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of any one of variants v23855, v29449, v29450, v29451, v29452, v29453, v29454, v29455, v29456, v29457, v29458, v29459, or v29460, where the antigen-binding domain retains the ability to bind hNaPi2b. In certain embodiments, the anti-NaPi2b antibody constructs of the present disclosure comprise an antigen-binding domain that comprises a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of any one of variants v23855, v29449, v29450, v29451, v29452, v29453, v29454, v29455, v29456, v29457, v29458, v29459, or v29460, where the antigen-binding domain retains the ability to bind hNaPi2b.

In certain embodiments, the anti-NaPi2b antibody constructs of the ADCs of the present disclosure comprise an antigen-binding domain comprising a VH amino acid sequence selected from the VH amino acid sequences of any one of variants v23855, v29449, v29450, v29451, v29452, v29453, v29454, v29455, v29456, v29457, v29458, v29459, or v29460. In certain embodiments, the anti-NaPi2b antibody constructs of the present disclosure comprise an antigen-binding domain comprising a VL amino acid sequence selected from the VL amino acid sequences of any one of variants v23855, v29449, v29450, v29451, v29452, v29453, v29454, v29455, v29456, v29457, v29458, v29459, or v29460.

In certain embodiments, the anti-NaPi2b antibody constructs of the ADCs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of v23855, and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of v23855, where the antigen-binding domain retains the ability to bind hNaPi2b.

In certain embodiments, the anti-NaPi2b antibody constructs of the ADCs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of v29456, and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of v29456, where the antigen-binding domain retains the ability to bind hNaPi2b.

In certain embodiments, the anti-NaPi2b antibody constructs of the ADCs of the present disclosure comprise an antigen-binding domain that comprises a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH sequence of v29452, and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL sequence of v29452, where the antigen-binding domain retains the ability to bind hNaPi2b.

In some embodiments, the anti-NaPi2b antibody construct of the ADCs of the present disclosure comprises the VH and VL sequences of any one of v23855, v29449, v29450, v29451, v29452, v29453, v29454, v29455, v29456, v29457, v29458, v29459, or v29460. The SEQ ID NOs: of the VH and VL sequences of these variants are provided below in Table 4. The sequences themselves are provided in Table 7.4 of the Examples.

TABLE 4
VH and VL sequences of parental
chimeric and humanized anti-NaPi2b
antibodies

| | VH sequence | VL sequence |
|---|---|---|
| Variant | (SEQ ID NO:) | (SEQ ID NO:) |
| 23855 | 31 | 32 |
| 29449 | 27 | 30 |
| 29450 | 26 | 30 |
| 29451 | 25 | 30 |
| 29452 | 24 | 30 |
| 29453 | 27 | 29 |
| 29454 | 26 | 29 |
| 29455 | 25 | 29 |
| 29456 | 24 | 29 |
| 29457 | 27 | 28 |
| 29458 | 26 | 28 |
| 29459 | 25 | 28 |
| 29460 | 24 | 28 |

In some embodiments, the anti-NaPi2b antibody construct of the ADC of the present disclosure comprises the VH sequence and the VL sequence of v29456. In some embodiments, the anti-NaPi2b antibody construct of the ADC of the present disclosure comprises the VH sequence and the VL sequence of v29452.

In certain embodiments, the anti-NaPi2b antibody construct of the ADC of the present disclosure comprises a) a VH sequence having the 3 HCDRs of v29456 and having at least at least 91%, at least 92%, at least 93%, at least 94%, at least 95%, at least 96%, at least 97%, at least 98%, or at least 99% identity to the VH sequence of v29456, and b) a VL sequence having the 3 LCDRs of v29456 and having at least at least 91%, at least 92%, at least 93%, at least 94%, at least 95%, at least 96%, at least 97%, at least 98%, or at least 99% identity to the VL sequence of v29456, wherein the HCDRs and LCDRs are defined by any one of the IMGT, Chothia, Kabat, Contact or AbM numbering systems.

In certain other embodiments, the anti-NaPi2b antibody construct of the ADC of the present disclosure comprise a) a VH sequence having the 3 HCDRs of v29452 and having at least at least 91%, at least 92%, at least 93%, at least 94%, at least 95%, at least 96%, at least 97%, at least 98%, or at least 99% identity to the VH sequence of v29452, and b) a VL sequence having the 3 LCDRs of v29452 and having at least at least 91%, at least 92%, at least 93%, at least 94%, at least 95%, at least 96%, at least 97%, at least 98%, or at least 99% identity to the VL sequence of v29452, wherein the HCDRs and LCDRs are defined by any one of the IMGT, Chothia, Kabat, Contact or AbM numbering systems.

In one embodiment, the anti-NaPi2b antibody construct of the ADCs of the present disclosure comprises two heavy chains having the amino acid sequence as set forth in SEQ ID NO:63 and two light chains having the amino acid sequence as set forth in SEQ ID NO:62. In one embodiment, the anti-NaPi2b antibody construct of the ADCs of the present disclosure comprises two heavy chains having the amino acid sequence as set forth in SEQ ID NO:61 and two light chains having the amino acid sequence as set forth in SEQ ID NO:62.

The anti-NaPi2b antibody construct of the ADCs of the present disclosure may comprise a silenced Fc region as described elsewhere herein (anti-NaPi2b Fc-silenced ADCs). In these embodiments, the anti-NaPi2b antibody construct of the ADCs of the present disclosure comprises two heavy chains having the amino acid sequence as set forth in SEQ ID NO:66 and two light chains having the amino acid sequence as set forth in SEQ ID NO:67. In yet other embodiments, the anti-NaPi2b antibody construct of the ADCs of the present disclosure comprises two heavy chains having the amino acid sequence as set forth in SEQ ID NO:68 and two light chains having the amino acid sequence as set forth in SEQ ID NO:67.

In additional embodiments, the anti-NaPi2b antibody construct of the anti-NaPi2b Fc-silenced ADC of the present disclosure comprises two heavy chains having (a) at least 90%, at least 91%, at least 92%, at least 93%, at least 94%, at least 95%, at least 96%, at least 97%, at least 98%, or at least 99% identity to the amino acid sequence as set forth in SEQ ID NO:66 and (b) two light chains having at least 90%, at least 91%, at least 92%, at least 93%, at least 94%, at least 95%, at least 96%, at least 97%, at least 98%, or at least 99% identity to the amino acid sequence as set forth in SEQ ID NO:67.

In still other embodiments, the anti-NaPi2b antibody construct of the anti-NaPi2b Fc-silenced ADC of the present disclosure comprises two heavy chains having (a) at least 91%, at least 92%, at least 93%, at least 94%, at least 95%, at least 96%, at least 97%, at least 98%, or at least 99% identity to the amino acid sequence as set forth in SEQ ID NO:68 and (b) two light chains having at least 90%, at least 91%, at least 92%, at least 93%, at least 94%, at least 95%, at least 96%, at least 97%, at least 98%, or at least 99% identity to the amino acid sequence as set forth in SEQ ID NO:67.

### Formats

The anti-NaPi2b antibody constructs of the ADC may have various formats. The minimal component of the anti-NaPi2b antibody construct is an antigen-binding domain that binds to hNaPi2b. The anti-NaPi2b antibody constructs may further optionally comprise one or more additional antigen-binding domains and/or a scaffold. In those embodiments in which the anti-NaPi2b antibody construct comprises two or more antigen-binding domains, each additional antigen-binding domain may bind to the same epitope within hNaPi2b, may bind to a different epitope within hNaPi2b, or may bind to a different antigen. Thus, the anti-NaPi2b antibody construct may be, for example, monospecific, biparatopic, bispecific or multispecific.

In certain embodiments, the anti-NaPi2b antibody construct comprises at least one antigen-binding domain that binds to hNaPi2b and a scaffold, where the antigen-binding domain is operably linked to the scaffold. The term “operably linked,” as used herein, means that the components described are in a relationship permitting them to function in their intended manner. Suitable scaffolds are described below.

In certain embodiments, the anti-NaPi2b antibody construct comprises two antigen-binding domains optionally operably linked to a scaffold. In some embodiments, the anti-NaPi2b antibody construct may comprise three or four antigen-binding domains and optionally a scaffold. In these formats, when comprising a scaffold, at least a first antigen-binding domain is operably linked to the scaffold and the remaining antigen-binding domain(s) may each independently be operably linked to the scaffold or to the first antigen-binding domain or, when more than two antigen-binding domains are present, to another antigen-binding domain.

Anti-NaPi2b antibody constructs that lack a scaffold may comprise a single antigen-binding domain in an appropriate format, such as an sdAb, or they may comprise two or more antigen-binding domains optionally operably linked by one or more linkers. In such anti-NaPi2b antibody constructs, the antigen-binding domains may be in the form of scFvs, Fabs, sdAbs, or a combination thereof. For example, using scFvs as the antigen-binding domains, formats such as a tandem scFv ((scFv)₂ or taFv) may be constructed, in which the scFvs are connected together by a flexible linker. scFvs may also be used to construct diabody formats, which comprise two scFvs connected by a short linker (usually about 5 amino acids in length). The restricted length of the linker results in dimerization of the scFvs in a head-to-tail manner. In any of the preceding formats, the scFvs may be further stabilized by inclusion of an interdomain disulfide bond. For example, a disulfide bond may be introduced between VL and VH through substitution of non-cysteine residues to cysteine residues in each chain (for example, at position 44 in VH and 100 in VL) (see, for example, Fitzgerald et al., 1997, Protein Engineering, 10:1221-1225), or a disulfide bond may be introduced between two VHs to provide a construct having a DART format (see, for example, Johnson et al., 2010, J Mol. Biol., 399:436-449).

Similarly, formats comprising two sdAbs, such as VHs or VHHs, connected together through a suitable linker may be employed in some embodiments. Other examples of anti-NaPi2b antibody construct formats that lack a scaffold include those based on Fab fragments, for example, Fab₂ and F(ab′)₂ formats, in which the Fab fragments are connected through a linker or an IgG hinge region.

Combinations of antigen-binding domains in different forms may also be employed to generate alternative scaffold-less formats. For example, an scFv or a sdAb may be fused to the C-terminus of either or both of the light and heavy chain of a Fab fragment resulting in a bivalent (Fab-scFv/sdAb) construct.

In certain embodiments, the anti-NaPi2b antibody construct may be in an antibody format that is based on an immunoglobulin (Ig). This type of format is referred to herein as a full-size antibody format (FSA) or Mab format and includes anti-NaPi2b antibody constructs that comprise two Ig heavy chains and two Ig light chains. In certain embodiments, the anti-NaPi2b antibody construct may be based on an IgG class immunoglobulin, for example, an IgG1, IgG2, IgG3 or IgG4 immunoglobulin. In some embodiments, the anti-NaPi2b antibody construct may be based on an IgG1 immunoglobulin. In the context of the present disclosure, when an anti-NaPi2b antibody construct is based on a specified immunoglobulin isotype, it is meant that the anti-NaPi2b antibody construct comprises all or a portion of the constant region of the specified immunoglobulin isotype. For example, an anti-NaPi2b antibody construct based on a given Ig isotype may comprise at least one antigen-binding domain operably linked to an Ig scaffold, where the scaffold comprises an Fc region from the given isotype and optionally an Ig hinge region from the same or a different isotype. It is to be understood that the anti-NaPi2b antibody constructs may also comprise hybrids of isotypes and/or subclasses in some embodiments. It is also to be understood that the Fc region and/or hinge region may optionally be modified to impart one or more desirable functional properties as is known in the art. Thus, in certain embodiments, the anti-NaPi2b antibody construct comprises a VH amino acid sequence fused to IgG1 constant domain amino acid sequences (i.e. CH1, hinge, CH2, CH3 amino acid sequences) and a VL amino acid sequence fused to kappa or lambda constant amino acid sequences domain (i.e. CL amino acid sequences). Exemplary amino acid sequences are provided in the Examples and Sequence Tables.

In some embodiments, the anti-NaPi2b antibody constructs may be derived from two or more immunoglobulins that are from different species, for example, the anti-NaPi2b antibody construct may be a chimeric antibody or a humanized antibody. The terms “chimeric antibody” and “humanized antibody” both refer generally to antibodies that combine immunoglobulin regions or domains from more than one species.

A “chimeric antibody” typically comprises at least one variable domain from a non-human antibody, such as a rabbit or rodent (for example, murine) antibody, and at least one constant domain from a human antibody. The human constant domain of a chimeric antibody need not be of the same isotype as the non-human constant domain it replaces. Chimeric antibodies are discussed, for example, in Morrison et al., 1984, Proc. Natl. Acad. Sci. USA, 81:6851-55, and U.S. Pat. No. 4,816,567.

A “humanized antibody” is a type of chimeric antibody that contains minimal sequence derived from a non-human antibody. Generally, humanized antibodies are human immunoglobulins (recipient antibody) in which residues from a hypervariable region of the recipient are replaced by residues from a hypervariable region of a non-human species (donor antibody), such as mouse, rat, rabbit or non-human primate, having the desired specificity and affinity for a target antigen. This technique for creating humanized antibodies is often referred to as “CDR grafting.”

In some instances, additional modifications are made to further refine antibody performance. For example, some framework region (FR) residues of the human immunoglobulin are replaced by corresponding non-human residues, or the humanized antibodies may comprise residues that are not found in either the recipient antibody or the donor antibody. In general, a variable domain in a humanized antibody will comprise all or substantially all of the hypervariable regions from a non-human immunoglobulin and all or substantially all of the FRs from a human immunoglobulin sequence. Humanized antibodies are described in more detail in Jones, et al., 1986, Nature, 321:522-525; Riechmann, et al., 1988, Nature, 332:323-329, and Presta, 1992, Curr. Op. Struct. Biol., 2:593-596, for example.

A number of approaches are known in the art for selecting the most appropriate human frameworks in which to graft the non-human CDRs. Early approaches used a limited subset of well-characterised human antibodies, irrespective of the sequence identity to the non-human antibody providing the CDRs (the “fixed frameworks” approach). More recent approaches have employed variable regions with high amino acid sequence identity to the variable regions of the non-human antibody providing the CDRs (“homology matching” or “best-fit” approach). An alternative approach is to select fragments of the framework sequences within each light or heavy chain variable region from several different human antibodies. CDR-grafting may in some cases result in a partial or complete loss of affinity of the grafted molecule for its target antigen. In such cases, affinity can be restored by back-mutating some of the residues of human origin to the corresponding non-human ones. Methods for preparing humanized antibodies by these approaches are well-known in the art (see, for example, Tsurushita & Vasquez, 2004, Humanization of Monoclonal Antibodies, Molecular Biology of B Cells, 533-545, Elsevier Science (USA); Jones et al., 1986, Nature, 321:522-525; Riechmann et al., 1988, Nature, 332:323-329; Presta et al., 1997, Cancer Res, 57(20):4593-4599).

Alternatively, or in addition to, these traditional approaches, more recent technologies may be employed to further reduce the immunogenicity of a CDR-grafted humanized antibody. For example, frameworks based on human germline sequences or consensus sequences may be employed as acceptor human frameworks rather than human frameworks with somatic mutation(s). Another technique that aims to reduce the potential immunogenicity of non-human CDRs is to graft only specificity-determining residues (SDRs). In this approach, only the minimum CDR residues required for antigen-binding activity (the “SDRs”) are grafted into a human germline framework. This method improves the “humanness” (i.e. the similarity to human germline sequence) of the humanized antibody and thus may help reduce the risk of immunogenicity of the variable region. These techniques have been described in various publications (see, for example, Almagro & Fransson, 2008, Front Biosci, 13:1619-1633; Tan, et al., 2002, J Immunol, 169:1119-1125; Hwang, et al., 2005, Methods, 36:35-42; Pelat, et al., 2008, J Mol Biol, 384:1400-1407; Tamura, et al., 2000, J Immunol, 164:1432-1441; Gonzales, et al., 2004, Mol Immunol, 1:863-872, and Kashmiri, et al., 2005, Methods, 36:25-34).

In certain embodiments, the anti-NaPi2b antibody construct of the present disclosure comprises humanized antibody sequences, for example, one or more humanized variable domains. In some embodiments, the anti-NaPi2b antibody construct can be a humanized antibody. Non-limiting examples of humanized antibodies based on the anti-NaPi2b antibody v23855 are described herein (see Examples and Sequence Tables and sequences for v29449, v29450, v29451, v29452, v29453, v29454, v29455, v29456, v29457, v29458, v29459, v29460, v38951, and v40502).

### Scaffolds

In certain embodiments, the anti-NaPi2b antibody constructs comprise one or more antigen-binding domains operably linked to a scaffold. The antigen-binding domain(s) may be in one or a combination of the forms described above (for example, scFvs, Fabs and/or sdAbs). Examples of suitable scaffolds are described in more detail below and include, but are not limited to, immunoglobulin Fc regions, albumin, albumin analogues and derivatives, heterodimerizing peptides (such as leucine zippers, heterodimer-forming “zipper” peptides derived from Jun and Fos, IgG CH1 and CL domains or barnase-barstar toxins), cytokines, chemokines or growth factors. Other examples include antibodies based on the DOCK-AND-LOCK™ (DNL™) technology developed by IBC Pharmaceuticals, Inc. and Immunomedics, Inc. (see, for example, Chang, et al., 2007, Clin. Cancer Res., 13:5586s-5591s).

A scaffold may be a peptide, polypeptide, polymer, nanoparticle or other chemical entity. Where the scaffold is a polypeptide, each antigen-binding domain of the anti-NaPi2b antibody construct may be linked to either the N- or C-terminus of the polypeptide scaffold. Anti-NaPi2b antibody construct comprising a polypeptide scaffold in which one or more of the antigen-binding polypeptide constructs are linked to a region other than the N- or C-terminus, for example, via the side chain of an amino acid with or without a linker, are also contemplated in certain embodiments.

In embodiments where the anti-NaPi2b antibody construct comprises a scaffold that is a peptide or polypeptide, the antigen-binding domain(s) may be linked to the scaffold by genetic fusion or chemical conjugation. Typically, when the scaffold is a peptide or polypeptide, the antigen-binding domain(s) are linked to the scaffold by genetic fusion. In some embodiments, where the scaffold is a polymer or nanoparticle, the antigen-binding domain(s) may be linked to the scaffold by chemical conjugation.

A number of protein domains are known in the art that comprise selective pairs of two different polypeptides and may be used to form a scaffold. An example is leucine zipper domains such as Fos and Jun that selectively pair together (Kostelny, et al., J Immunol, 148:1547-53 (1992); Wranik, et al., J. Biol. Chem., 287: 43331-43339 (2012)). Other selectively pairing molecular pairs include, for example, the barnase-barstar pair (Deyev, et al., Nat Biotechnol, 21:1486-1492 (2003)), DNA strand pairs (Chaudri, et al., FEBS Letters, 450(1-2):23-26 (1999)) and split fluorescent protein pairs (International Patent Application Publication No. WO 2011/135040).

Other examples of protein scaffolds include immunoglobulin Fc regions, albumin, albumin analogues and derivatives, toxins, cytokines, chemokines and growth factors. The use of protein scaffolds in combination with antigen-binding moieties has been described (see, for example, Müller et al., 2007, J. Biol. Chem., 282:12650-12660; McDonaugh et al., 2012, Mol. Cancer Ther., 11:582-593; Vallera et al., 2005, Clin. Cancer Res., 11:3879-3888; Song et al., 2006, Biotech. Appl. Biochem., 45:147-154, and U.S. Patent Application Publication No. 2009/0285816).

For example, fusing antigen-binding moieties such as scFvs, diabodies or single chain diabodies to albumin has been shown to improve the serum half-life of the antigen-binding moieties (Müller et al., ibid.). Antigen-binding moieties may be fused at the N- and/or C-termini of albumin, optionally via a linker.

Derivatives of albumin in the form of heteromultimers that comprise two transporter polypeptides obtained by segmentation of an albumin protein such that the transporter polypeptides self-assemble to form quasi-native albumin have been described (see International Patent Application Publication Nos. WO 2012/116453 and WO 2014/012082). As a result of the segmentation of albumin, the heteromultimer includes four termini and thus can be fused to up to four different antigen-binding moieties, optionally via linkers.

In certain embodiments, the anti-NaPi2b antibody construct may comprise a protein scaffold. In some embodiments, the anti-NaPi2b antibody construct may comprise a protein scaffold that is based on an immunoglobulin Fc region, an albumin or an albumin analogue or derivative. In some embodiments, the anti-NaPi2b antibody construct may comprise a protein scaffold that is based on an immunoglobulin Fc region, for example, an IgG Fc region.

### Fc Regions

The terms “Fc region,” “Fc” or “Fc domain” as used herein refer to a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, MD (1991).

In certain embodiments, the anti-NaPi2b antibody constructs may comprise a scaffold that is based on an immunoglobulin Fc region. The Fc region may be dimeric and composed of two Fc polypeptides or alternatively, the Fc region may be composed of a single polypeptide.

An “Fc polypeptide” in the context of a dimeric Fc refers to one of the two polypeptides forming the dimeric Fc domain, i.e. a polypeptide comprising one or more C-terminal constant regions of an immunoglobulin heavy chain that is capable of stable self-association. When referring to a dimeric Fc region, the terms “first Fc polypeptide” and “second Fc polypeptide” may be used interchangeably provided that the Fc region comprises one first Fc polypeptide and one second Fc polypeptide.

An Fc region may comprise a CH3 domain or it may comprise both a CH3 and a CH2 domain. For example, in certain embodiments, an Fc polypeptide of a dimeric IgG Fc region may comprise an IgG CH2 domain sequence and an IgG CH3 domain sequence. In such embodiments, the CH3 domain comprises two CH3 sequences, one from each of the two Fc polypeptides of the dimeric Fc region, and the CH2 domain comprises two CH2 sequences, one from each of the two Fc polypeptides of the dimeric Fc region.

In some embodiments, the anti-NaPi2b antibody construct may comprise a scaffold that is based on an IgG Fc region. In some embodiments, the anti-NaPi2b antibody construct may comprise a scaffold that is based on a human IgG Fc region. In some embodiments, the anti-NaPi2b antibody construct may comprise a scaffold based on an IgG1 Fc region. In some embodiments, the anti-NaPi2b antibody construct may comprise a scaffold based on a human IgG1 Fc region.

In certain embodiments, the anti-NaPi2b antibody construct may comprise a scaffold based on an IgG Fc region, which is a heterodimeric Fc region, comprising a first Fc polypeptide and a second Fc polypeptide, each comprising a CH3 sequence, and optionally a CH2 sequence and in which the first and second Fc polypeptides are different. In some embodiments, the anti-NaPi2b antibody construct may comprise a scaffold based on an Fc region which comprises two CH3 sequences, at least one of which comprises one or more amino acid modifications. In some embodiments, the anti-NaPi2b antibody construct may comprise a scaffold based on an Fc region which comprises two CH3 sequences and two CH2 sequences, at least one of the CH2 sequences comprising one or more amino acid modifications.

In some embodiments, the anti-NaPi2b antibody construct may comprise a heterodimeric Fc region comprising a modified CH3 domain, where the modified CH3 domain is an asymmetrically modified CH3 domain comprising one or more asymmetric amino acid modifications. As used herein, an “asymmetric amino acid modification” refers to a modification, such as a substitution or an insertion, in which an amino acid at a specific position on a first CH3 or CH2 sequence is different to the amino acid on a second CH3 or CH2 sequence at the same position. These asymmetric amino acid modifications can be a result of modification of only one of the two amino acids at the same respective amino acid position on each sequence, or different modifications of both amino acids on each sequence at the same respective position on each of the first and second CH3 or CH2 sequences. Each of the first and second CH3 or CH2 sequences of a heterodimeric Fc may comprise one or more than one asymmetric amino acid modification.

In some embodiments, the anti-NaPi2b antibody construct may comprise a heterodimeric Fc comprising a modified CH3 domain, where the modified CH3 domain comprises one or more amino acid modifications that promote formation of the heterodimeric Fc over formation of a homodimeric Fc. In some embodiments, one or more of the amino acid modifications are asymmetric amino acid modifications.

Amino acid modifications that may be made to the CH3 domain of an Fc in order to promote formation of a heterodimeric Fc are known in the art and include, for example, those described in International Publication No. WO 96/027011 (“knobs into holes”), Gunasekaran et al., 2010, J Biol Chem, 285, 19637-46 (“electrostatic steering”), Davis et al., 2010, Prot Eng Des Sel, 23(4):195-202 (strand exchange engineered domain (SEED) technology) and Labrijn et al., 2013, Proc Natl Acad Sci USA, 110(13):5145-50 (Fab-arm exchange). Other examples include approaches combining positive and negative design strategies to produce stable asymmetrically modified Fc regions as described in International Publication Nos. WO 2012/058768 and WO 2013/063702. In certain embodiments, the anti-NaPi2b antibody construct may comprise a scaffold based on a modified Fc region as described in International Publication No. WO 2012/058768 or WO 2013/063702.

Table 5 provides the amino acid sequence of the human IgG1 Fc sequence (SEQ ID NO:60), corresponding to amino acids 231 to 447 of the full-length human IgG1 heavy chain. The CH3 sequence comprises amino acids 341-447 of the full-length human IgG1 heavy chain. Also shown in Table 5 are CH3 domain amino acid modifications that promote formation of a heterodimeric Fc as described in in International Patent Application Publication Nos. WO 2012/058768 and WO 2013/063702.

In certain embodiments, the anti-NaPi2b antibody construct may comprise a heterodimeric Fc scaffold having a modified CH3 domain comprising the modifications of any one of Variant 1, Variant 2, Variant 3, Variant 4 or Variant 5, as shown in Table 5.

TABLE 5
Human IgG1 Fc Sequence¹ and CH3 Domain Amino Acid Modifications
Promoting Heterodimer Formation
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK (SEQ ID NO: 60)

| Variant No | Chain | Mutations |
|---|---|---|
| 1 | A | L351Y F405A Y407V |
| | B | T366L K392M T394W |
| 2 | A | L351Y F405A Y407V |
| | B | T366L K392L T394W |
| 3 | A | T350V L351Y F405A Y407V |
| | B | T350V T366L K392L T394W |
| 4 | A | T350V L351Y F405A Y407V |
| | B | T350V T366L K392M T394W |
| 5 | A | T350V L351Y S400E F405A Y407V |
| | B | T350V T366L N390R K392M T394W |

¹Sequence from positions 231-447 (EU numbering)

In some embodiments, the anti-NaPi2b antibody construct may comprise a scaffold based on an Fc region comprising two CH3 sequences and two CH2 sequences, at least one of the CH2 sequences comprising one or more amino acid modifications. Modifications in the CH2 domain can affect the binding of Fc receptors (FcRs) to the Fc, such as receptors of the FcγRI, FcγRII and FcγRIII subclasses.

In some embodiments, the anti-NaPi2b antibody construct comprises a scaffold based on an IgG Fc having a modified CH2 domain, wherein the modification of the CH2 domain results in altered binding to one or more of the FcγRI, FcγRII and FcγRIII receptors.

A number of amino acid modifications to the CH2 domain that selectively alter the affinity of the Fc for different Fcγ receptors are known in the art. Amino acid modifications that result in increased binding and amino acid modifications that result in decreased binding can each be useful in certain indications. For example, increasing binding affinity of an Fc for FcγRIIIa (an activating receptor) may result in increased antibody dependent cell-mediated cytotoxicity (ADCC), which in turn results in increased lysis of the target cell. Decreased binding to FcγRIIb (an inhibitory receptor) likewise may be beneficial in some circumstances. In certain indications, a decrease in, or elimination of, ADCC and complement-mediated cytotoxicity (CDC) may be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) may be useful.

In certain embodiments of the ADCs described herein, anti-NaPi2b antibody constructs having amino acid modifications in the Fc that ablate binding to Fcγ receptors may provide benefits related to decreasing toxicity of the ADC compared to ADCs having an anti-NaPi2b antibody construct with a wild-type Fc region (see Kumagai, et al., 2020, Cancer Science, 111:4636-4645).

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365(1-2):132-41); F243L/R292P/Y300L/V305I/P396L (increased affinity for FcγRIIIa) (Stavenhagen, et al., 2007, Cancer Res, 67(18):8882-90); F243L/R292P/Y300L/L235V/P396L (increased affinity for FcγRIIIa) (Nordstrom J L, et al., 2011, Breast Cancer Res, 13(6):R123); F243L (increased affinity for FcγRIIIa) (Stewart, et al., 2011, Protein Eng Des Sel., 24(9):671-8); S298A/E333A/K334A (increased affinity for FcγRIIIa) (Shields, et al., 2001, J Biol Chem, 276(9):6591-604); S239D/I332E/A330L and S239D/I332E (increased affinity for FcγRIIIa) (Lazar, et al., 2006, Proc Natl Acad Sci USA, 103(11):4005-10), and S239D/S267E and S267E/L328F (increased affinity for FcγRIIb) (Chu, et al., 2008, Mol Immunol, 45(15):3926-33). Various amino acid modifications to the CH2 domain that alter binding of the Fc by FcγRIIb are described in International Publication No. WO 2021/232162. Additional modifications that affect Fc binding to Fcγ receptors are described in Therapeutic Antibody Engineering (Strohl & Strohl, Woodhead Publishing series in Biomedicine No 11, ISBN 1 907568 37 9, Oct 2012, page 283).

In certain embodiments, the anti-NaPi2b antibody construct comprises a scaffold based on an IgG Fc having a modified CH2 domain, in which the modified CH2 domain comprises one or more amino acid modifications that result in decreased or eliminated binding of the Fc region to all of the Fcγ receptors (i.e. a “knock-out” variant).

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation, use of IgG2/IgG4 scaffolds, or the introduction of mutations in the hinge or CH2 domain of the Fc (see also, U.S. Patent Publication No. 2011/0212087, International Publication No. WO 2006/105338, U.S. Patent Publication No. 2012/0225058, U.S. Patent Publication No. 2012/0251531 and Strop et al., 2012, J. Mol. Biol., 420: 204-219).

Examples of mutations that may be introduced into the hinge or CH2 domain to produce a “knock-out” variant include the amino acid modifications L234A/L235A (LALA), and L234A/L235A/D265S (LALADS). LALA substitutions in IgG1 antibodies have been shown to reduce binding of the antibody to FcγRs (Wilkinson et al. PLoS One. 2021; 16(12)), thus disrupting, but not completely knocking out, the ability of these antibodies to interact with certain cells in tissues. In general, antibodies having an Fc region comprising the LALADS modifications are Fc-silenced since they do not exhibit ADCC or ADCP (antibody-dependent cellular phagocytosis), and do not bind C1q. The LALADS modifications are described in International Patent Publication No. WO 2014/012085.

In certain embodiments, the anti-NaPi2b antibody constructs described herein may comprise a scaffold based on an IgG Fc in which native glycosylation has been modified. As is known in the art, glycosylation of an Fc may be modified to increase or decrease effector function. For example, mutation of the conserved asparagine residue at position 297 to alanine, glutamine, lysine or histidine (i.e. N297A, Q, K or H) results in an aglycoslated Fc that lacks all effector function (Bolt et al., 1993, Eur. J Immunol., 23:403-411; Tao & Morrison, 1989, J. Immunol., 143:2595-2601).

Conversely, removal of fucose from heavy chain N297-linked oligosaccharides has been shown to enhance ADCC, based on improved binding to FcγRIIIa (see, for example, Shields et al., 2002, J Biol Chem., 277:26733-26740, and Niwa et al., 2005, J. Immunol. Methods, 306:151-160). Such low fucose antibodies may be produced, for example in knockout Chinese hamster ovary (CHO) cells lacking fucosyltransferase (FUT8) (Yamane-Ohnuki et al., 2004, Biotechnol. Bioeng., 87:614-622); in the variant CHO cell line, Lec 13, that has a reduced ability to attach fucose to N297-linked carbohydrates (International Publication No. WO 03/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogues to culture medium during antibody production to inhibit incorporation of fucose into the carbohydrate on the antibody.

Other methods of producing antibodies with little or no fucose on the Fc glycosylation site (N297) are well known in the art. For example, the GlymaX® technology (ProBioGen AG) (see von Horsten et al., 2010, Glycobiology, 20(12):1607-1618 and U.S. Pat. No. 8,409,572).

Other glycosylation variants include those with bisected oligosaccharides, for example, variants in which a biantennary oligosaccharide attached to the Fc region of the antibody is bisected by N-acetylglucosamine (GlcNAc). Such glycosylation variants may have reduced fucosylation and/or improved ADCC function (see, for example, International Publication No. WO 2003/011878, U.S. Pat. No. 6,602,684 and US Patent Application Publication No. US 2005/0123546). Useful glycosylation variants also include those having at least one galactose residue in the oligosaccharide attached to the Fc region, which may have improved CDC function (see, for example, International Publication Nos. WO 1997/030087, WO 1998/58964 and WO 1999/22764).

### Anti-NaPi2b Antibody Constructs Having a Silenced Fc

In one embodiment, the anti-NaPi2b antibody construct of an ADC of this disclosure is an Fc-silenced anti-NaPi2b antibody construct comprising LALADS amino acid substitutions in the Fc region. In some embodiments, the Fc-silenced anti-NaPi2b antibody construct comprising LALADS amino acid substitutions in the Fc region binds to NaPi2b-expressing cancer cells, and/or is internalized by NaPi2b-expressing cancer cells. In some embodiments, target-independent binding, i.e., the binding to cells that do not (or only minimally) express NaPi2b, such as the binding of the Fc region to one or more Fc receptors of non-target cells (e.g., macrophages), of the Fc-silenced anti-NaPi2b ADCs of the present disclosure is lower compared to the binding of comparable Fc WT anti-NaPi2b ADCs. In some embodiments, the Fc-silenced anti-NaPi2b antibody construct comprising LALADS amino acid substitutions in the Fc region exhibits reduced binding to one or more Fcγ receptors when compared to a reference anti-NaPi2b antibody construct that comprises a wildtype Fc region (e.g., an Fc region that does not comprise LALADS amino acid substitutions). In some embodiments, the Fc-silenced anti-NaPi2b antibody construct comprising LALADS amino acid substitutions in the Fc region exhibits reduced ADCC and/or ADCP when compared to a reference anti-NaPi2b antibody construct that comprises a wildtype Fc region (e.g., an Fc region that does not comprise LALADS amino acid substitutions). Without being bound by any theory, the reduced ADCC and/or ADCP as well as the reduced binding to NaPi2b-negative cells via the Fc region of the Fc-silenced anti-NaPi2b antibody construct comprising LALADS amino acid substitutions in the Fc region may indicate reduced off-target cell engagement and may translate into the potential for reduced toxicity in vivo. Hence, in some embodiments, Fc-silencing of an ADC of the present disclosure can have the potential to minimize toxicities driven by target-independent cellular uptake via FcγRs.

### Preparation of Anti-NaPi2b Antibody Constructs

The anti-NaPi2b antibody constructs described herein may be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

Typically, for recombinant production of an antibody construct, a polynucleotide or set of polynucleotides encoding the anti-NaPi2b antibody construct is generated and inserted into one or more vectors for further cloning and/or expression in a host cell. Polynucleotide(s) encoding the anti-NaPi2b antibody construct may be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the anti-NaPi2b antibody construct will be dependent on the format of the construct, including whether or not the antibody construct comprises a scaffold. For example, when an anti-NaPi2b antibody construct is in a monospecific mAb format or FSA format, two polynucleotides each encoding one polypeptide chain will be required. When multiple polynucleotides are required, they may be incorporated into one vector or into more than one vector.

Generally, for expression, the polynucleotide or set of polynucleotides is incorporated into an expression vector or vectors together with one or more regulatory elements, such as transcriptional elements, which are required for efficient transcription of the polynucleotide. Examples of such regulatory elements include, but are not limited to, promoters, enhancers, terminators, and polyadenylation signals. One skilled in the art will appreciate that the choice of regulatory elements is dependent on the host cell selected for expression of the antibody construct and that such regulatory elements may be derived from a variety of sources, including bacterial, fungal, viral, mammalian or insect genes. The expression vector may optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector may be an extrachromosomal vector or an integrating vector.

Suitable host cells for cloning or expression of the anti-NaPi2b antibody constructs include various prokaryotic or eukaryotic cells as known in the art. Eukaryotic host cells include, for example, mammalian cells, plant cells, insect cells and yeast cells (such as Saccharomyces or Pichia cells). Prokaryotic host cells include, for example, E. coli, A. salmonicida or B. subtilis cells.

In certain embodiments, the anti-NaPi2b antibody construct may be produced in bacteria, in particular when glycosylation and Fc effector function are not needed, as described for example in U.S. Pat. Nos. 5,648,237; 5,789,199, and 5,840,523, and in Charlton, Methods in Molecular Biology, Vol. 248, pp. 245-254, B. K. C. Lo, ed., Humana Press, Totowa, N.J., 2003.

Eukaryotic microbes such as filamentous fungi or yeast may be suitable expression host cells in certain embodiments, in particular fungi and yeast strains whose glycosylation pathways have been “humanized” resulting in the production of an antibody construct with a partially or fully human glycosylation pattern (see, for example, Gerngross, 2004., Nat. Biotech. 22:1409-1414, and Li et al., 2006, Nat. Biotech. 24:210-215).

Suitable host cells for the expression of glycosylated anti-NaPi2b antibody constructs are usually eukaryotic cells. For example, U.S. Pat. Nos. 5,959,177, 6,040,498, 6,420,548, 7,125,978 and 6,417,429 describe PLANTIBODIES™ technology for producing antigen-binding constructs in transgenic plants. Mammalian cell lines adapted to grow in suspension may be particularly useful for expression of antibody constructs. Examples include, but are not limited to, monkey kidney CV1 line transformed by SV40 (COS-7), human embryonic kidney (HEK) line 293 or 293 cells (see, for example, Graham et al., 1977, J. Gen Virol., 36:59), baby hamster kidney cells (BHK), mouse sertoli TM4 cells (see, for example, Mather, 1980, Biol Reprod, 23:243-251), monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR⁻ CHO cells, see Urlaub et al., 1980, Proc Natl Acad Sci USA, 77:4216), and myeloma cell lines (such as Y0, NS0 and Sp2/0). Exemplary mammalian host cell lines suitable for production of antibody constructs are reviewed in Yazaki & Wu, Methods in Molecular Biology, Vol. 248, pp. 255-268 (B. K. C. Lo, ed., Humana Press, Totowa, N.J., 2003).

In certain embodiments, the host cell may be a transient or stable higher eukaryotic cell line, such as a mammalian cell line. In some embodiments, the host cell may be a mammalian HEK293T, CHO, HeLa, NS0 or COS cell line, or a cell line derived from any one of these cell lines. In some embodiments, the host cell may be a stable cell line that allows for mature glycosylation of the antibody construct.

The host cells comprising the expression vector(s) encoding the anti-NaPi2b antibody construct may be cultured using routine methods to produce the anti-NaPi2b antibody construct. Alternatively, in some embodiments, host cells comprising the expression vector(s) encoding the anti-NaPi2b antibody construct may be used therapeutically or prophylactically to deliver the anti-NaPi2b antibody construct to a subject, or polynucleotides or expression vectors may be administered to a cell from a subject ex vivo and the cell then returned to the body of the subject.

Typically, the anti-NaPi2b antibody constructs are purified after expression. Proteins may be isolated or purified in a variety of ways known to those skilled in the art (see, for example, Protein Purification: Principles and Practice, 3^{rd} Ed., Scopes, Springer-Verlag, NY, 1994). Standard purification methods include chromatographic techniques, including ion exchange, hydrophobic interaction, affinity, sizing or gel filtration, and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, are also useful. As is well known in the art, a variety of natural proteins bind Fc and antibodies, and these proteins may be used for purification of certain antibody constructs. For example, the bacterial proteins A and G bind to the Fc region. Likewise, the bacterial protein L binds to the Fab region of some antibodies. Purification may also be enabled by a particular fusion partner. For example, antibodies may be purified using glutathione resin if a GST fusion is employed, Ni⁺² affinity chromatography if a His-tag is employed or immobilized anti-flag antibody if a flag-tag is used. The degree of purification necessary will vary depending on the use of the anti-NaPi2b antibody constructs. In some instances, no purification may be necessary.

In certain embodiments, the anti-NaPi2b antibody constructs are substantially pure. The term “substantially pure” (or “substantially purified”) when used in reference to an anti-NaPi2b antibody construct described herein, means that the antibody construct is substantially or essentially free of components that normally accompany or interact with the protein as found in its naturally occurring environment, such as a native cell, or a host cell in the case of recombinantly produced construct. In certain embodiments, an anti-NaPi2b antibody construct that is substantially pure is a protein preparation having less than about 30%, less than about 25%, less than about 20%, less than about 15%, less than about 10%, or less than about 5% (by dry weight) of contaminating protein.

Certain embodiments of the present disclosure relate to a method of making an anti-NaPi2b antibody construct comprising culturing a host cell into which one or more polynucleotides encoding the anti-NaPi2b antibody construct, or one or more expression vectors encoding the anti-NaPi2b antibody construct, have been introduced, under conditions suitable for expression of the anti-NaPi2b antibody construct, and optionally recovering the anti-NaPi2b antibody construct from the host cell (or from host cell culture medium).

### Post-Translational Modifications

In certain embodiments, the anti-NaPi2b antibody constructs described herein may comprise one or more post-translational modifications. Such post-translational modifications may occur in vivo, or they be conducted in vitro after isolation of the anti-NaPi2b antibody construct from the host cell.

Post-translational modifications include various modifications as are known in the art (see, for example, Proteins—Structure and Molecular Properties, 2nd Ed., T. E. Creighton, W. H. Freeman and Company, New York, 1993; Post-Translational Covalent Modification of Proteins, B. C. Johnson, Ed., Academic Press, New York, pgs. 1-12, 1983; Seifter et al., 1990, Meth. Enzymol., 182:626-646, and Rattan et al., 1992, Ann. N.Y. Acad. Sci., 663:48-62). In those embodiments in which the anti-NaPi2b antibody constructs comprise one or more post-translational modifications, the constructs may comprise the same type of modification at one or several sites, or it may comprise different modifications at different sites.

Examples of post-translational modifications include glycosylation, acetylation, phosphorylation, amidation, derivatization by known protecting/blocking groups, formylation, oxidation, reduction, proteolytic cleavage or specific chemical cleavage by cyanogen bromide, trypsin, chymotrypsin, papain, V8 protease or NaBH₄.

Other examples of post-translational modifications include, for example, addition or removal of N-linked or O-linked carbohydrate chains, chemical modifications of N-linked or O-linked carbohydrate chains, processing of N-terminal or C-terminal ends, attachment of chemical moieties to the amino acid backbone, and addition or deletion of an N-terminal methionine residue resulting from prokaryotic host cell expression. Post-translational modifications may also include modification with a detectable label, such as an enzymatic, fluorescent, luminescent, isotopic or affinity label to allow for detection and isolation of the protein. Examples of suitable enzyme labels include, but are not limited to, horseradish peroxidase, alkaline phosphatase, beta-galactosidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride and phycoerythrin. Examples of luminescent materials include luminol, and bioluminescent materials such as luciferase, luciferin and aequorin. Examples of suitable radioactive materials include iodine, carbon, sulfur, tritium, indium, technetium, thallium, gallium, palladium, molybdenum, xenon and fluorine.

Additional examples of post-translational modifications include acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, gamma-carboxylation, GPI anchor formation, hydroxylation, iodination, methylation, myristylation, pegylation, prenylation, racemization, selenoylation, sulfation, transfer-RNA mediated addition of amino acids to proteins such as arginylation, and ubiquitination.

### Camptothecin Analogues

The camptothecin analogue comprised by the ADCs of the present disclosure is a compound having Formula (I):

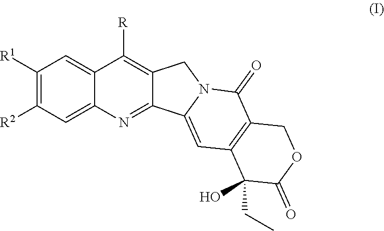

wherein:
R¹ is selected from: —H, —CH₃, —CHF₂, —CF₃, —F, —Br, —Cl, —OH, —OCH₃, —OCF₃ and —NH₂, and
R² is selected from: —H, —CH₃, —CF₃, —F, —Br, —Cl, —OH, —OCH₃ and —OCF₃, and wherein:
when R¹ is —NH₂, then R is R³ or R⁴, and when R¹ is other than —NH₂, then R is R⁴;
R³ is selected from: —H, —C₁-C₆ alkyl, —C₃-C₈ cycloalkyl, —(C₁-C₆ alkyl)-O—R⁵,

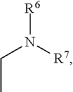

—CO₂R⁸, -aryl, -heteroaryl and —(C₁-C₆ alkyl)-aryl;
R⁴ is selected from:

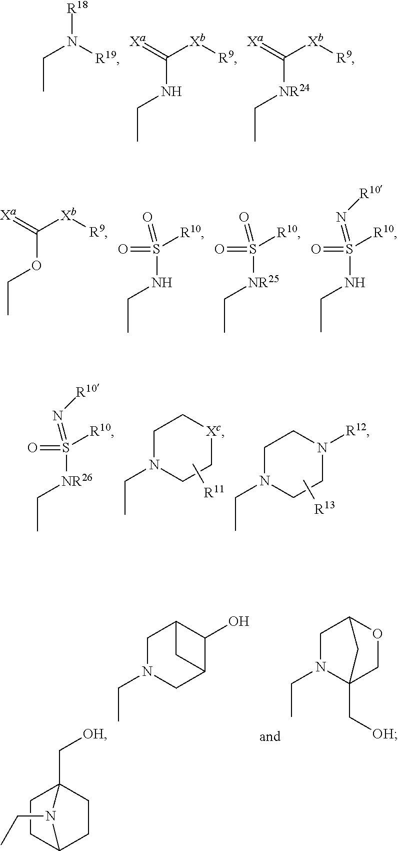

R⁵ is selected from: —H, —C₁-C₆ alkyl, —C₃-C₈ cycloalkyl, -aryl, -heteroaryl, -aryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
R^{10′} is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

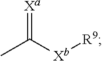

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from; O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

In some embodiments, the camptothecin analogues are compounds of Formula (I), with the proviso that when R¹ is NH₂, R² is other than H.

In some embodiments, in compounds of Formula (I), R¹ is selected from: —CH₃, —CF₃, —OCH₃, —OCF₃ and NH₂.

In some embodiments, in compounds of Formula (I), R¹ is NH₂.

In some embodiments, in compounds of Formula (I), R¹ is selected from: —H, —CH₃, —CF₃, —F, —Br, —Cl, —OH, —OCH₃ and —OCF₃.

In some embodiments, in compounds of Formula (I), R¹ is selected from: —CH₃, —CF₃, —OCH₃ and —OCF₃.

In some embodiments, in compounds of Formula (I), R² is selected from: —H, —CH₃, —CF₃, —F, —Cl, —OCH₃ and —OCF₃.

In some embodiments, in compounds of Formula (I), R² is selected from: —CH₃, —CF₃, —F, —Cl, —OCH₃ and —OCF₃.

In some embodiments, in compounds of Formula (I), R² is selected from: —H, —F, —Br and —Cl.

In some embodiments, in compounds of Formula (I), R² is selected from: —F, —Br and —Cl.

In some embodiments, in compounds of Formula (I), R³ is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

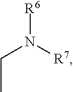

—CO₂R⁸, unsubstituted -aryl, -aminoaryl, -heteroaryl and —(C₁-C₆ alkyl)-aminoaryl.

In some embodiments, in compounds of Formula (I), R⁴ is selected from:

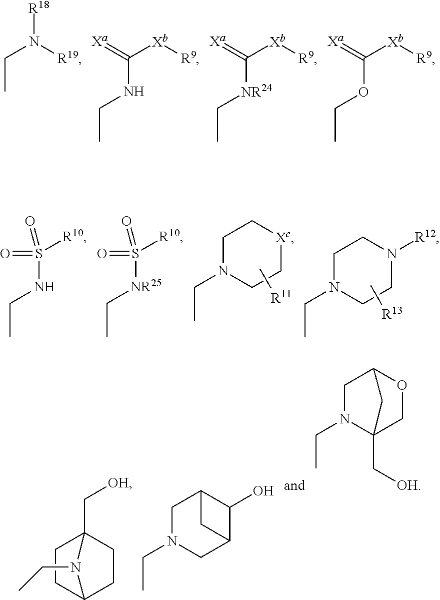

In some embodiments, in compounds of Formula (I), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (I), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (I), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (I), each R⁹ is independently selected from: —C₁-C₆ alkyl, —C₃-C₈ cycloalkyl, -aryl and —(C₁-C₆ alkyl)-aryl.

In some embodiments, in compounds of Formula (I), each R⁹ is independently selected from: —C₁-C₆ alkyl and —(C₁-C₆ alkyl)-aryl.

In some embodiments, in compounds of Formula (I), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (I), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (I), each R¹⁰ is independently selected from: —C₁-C₆ alkyl, —NR¹⁴R^{14′}, -aryl and —(C₁-C₆ alkyl)-aryl.

In some embodiments, in compounds of Formula (I), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (I), R^{10′} is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (I), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (I), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (I), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

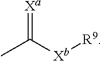

In some embodiments, in compounds of Formula (I), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (I), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (I), R¹⁶ is selected from: -aryl, -heteroaryl and —(C₁-C₆ alkyl)-aryl.

In some embodiments, in compounds of Formula (I), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted aryl, -aminoaryl, -heteroaryl and —(C₁-C₆ alkyl)-aminoaryl.

In some embodiments, in compounds of Formula (I), R¹⁷ is selected from: unsubstituted C₁-C₆ alkyl, —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted aryl, -hydroxyaryl, -aminoaryl, -heteroaryl and —(C₁-C₆ alkyl)-aminoaryl.

In some embodiments, in compounds of Formula (I), R¹⁸ and R¹⁹ taken together with the N atom to which they are bonded form a 4-, 5-, 6- or 7-membered ring having 0 to 3 substituents selected from: halogen, unsubstituted C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (I), X^{a} and X^{b} are each independently selected from: NH and O.

Combinations of any of the foregoing embodiments for compounds of Formula (I) are also contemplated and each combination forms a separate embodiment for the purposes of the present disclosure.

In certain embodiments, the compound of Formula (I) has Formula (II):

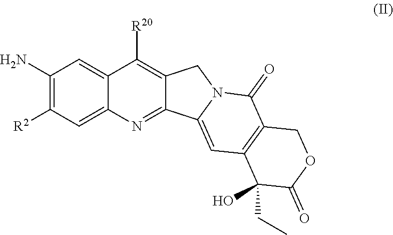

wherein:
R² is selected from: —H, —CH₃, —CF₃, —F, —Br, —Cl, —OH, —OCH₃ and —OCF₃;
R²⁰ is selected from: —H, —C₁-C₆ alkyl, —C₃-C₈ cycloalkyl, —(C₁-C₆ alkyl)-O—R⁵,

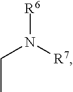

—CO₂R⁸, -aryl, -heteroaryl, —(C₁-C₆ alkyl)-aryl,

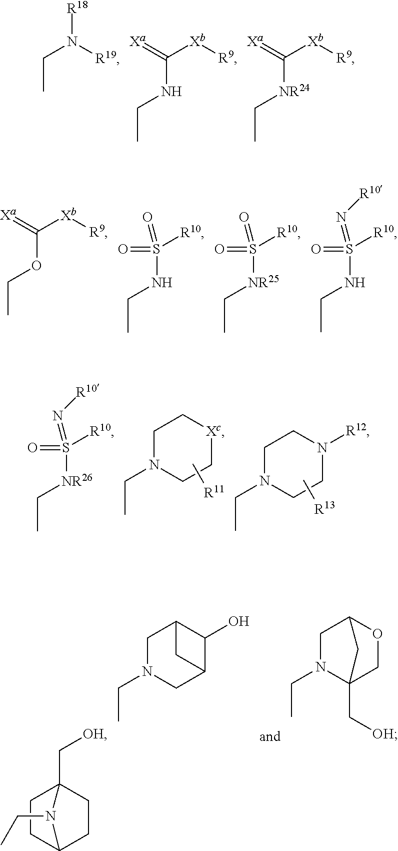

R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
R^{10′} is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

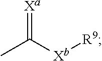

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from: O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

In some embodiments, in compounds of Formula (II), R² is selected from: —CH₃, —CF₃, —F, —Br, —Cl, —OH, —OCH₃ and —OCF₃.

In some embodiments, in compounds of Formula (II), R² is selected from: —CH₃, —CF₃, —F, —Cl, —OCH₃ and —OCF₃.

In some embodiments, in compounds of Formula (II), R² is selected from F and Cl.

In some embodiments, in compounds of Formula (II), R²⁰ is selected from: —H, —C₁-C₆ alkyl, —(C₁-C₆ alkyl)-O—R⁵,

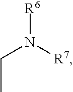

—(C₁-C₆ alkyl)-aryl,

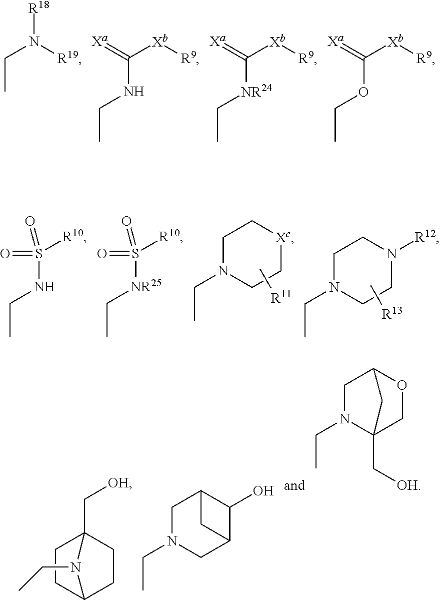

In some embodiments, in compounds of Formula (II), R²⁰ is selected from: —H, —C₁-C₆ alkyl, —(C₁-C₆ alkyl)-O—R⁵,

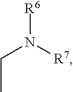

—(C₁-C₆ alkyl)-aryl,

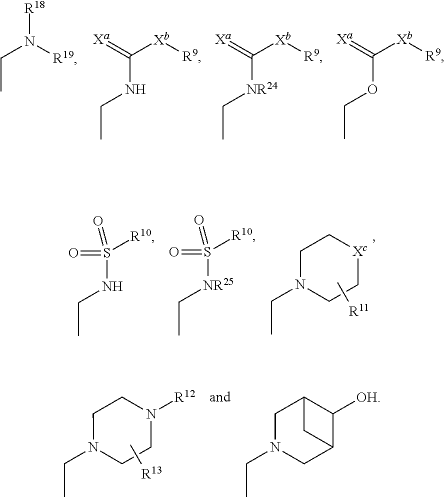

In some embodiments, in compounds of Formula (II), R²⁰ is selected from: —H, —C₁-C₆ alkyl, —(C₁-C₆ alkyl)-O—R⁵,

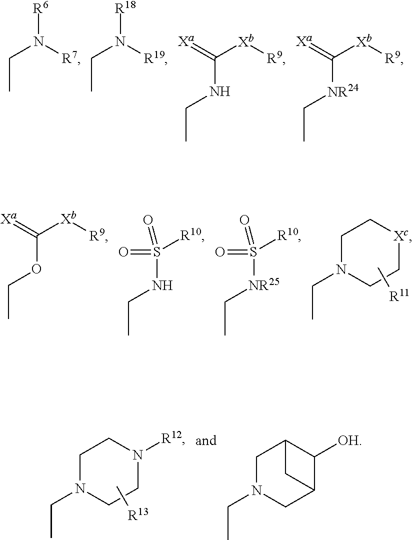

In some embodiments, in compounds of Formula (II), R²⁰ is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

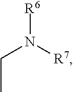

—CO₂R⁸, unsubstituted aryl, -aminoaryl, -heteroaryl, —(C₁-C₆ alkyl)-aminoaryl,

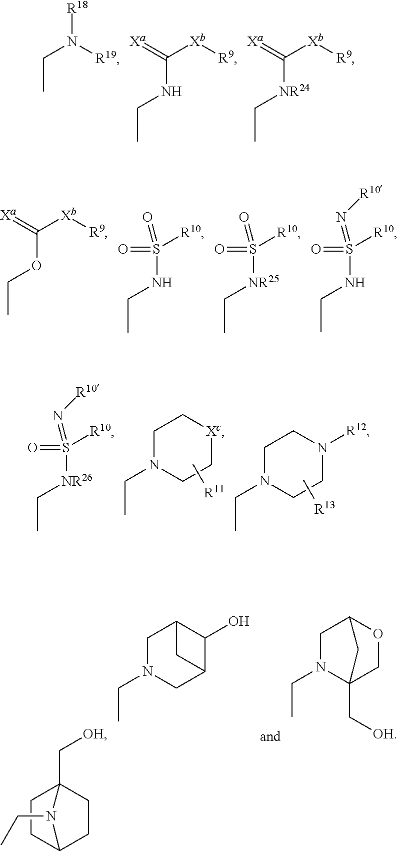

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—R⁵,

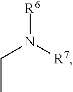

—(C₁-C₆ alkyl)-aryl,

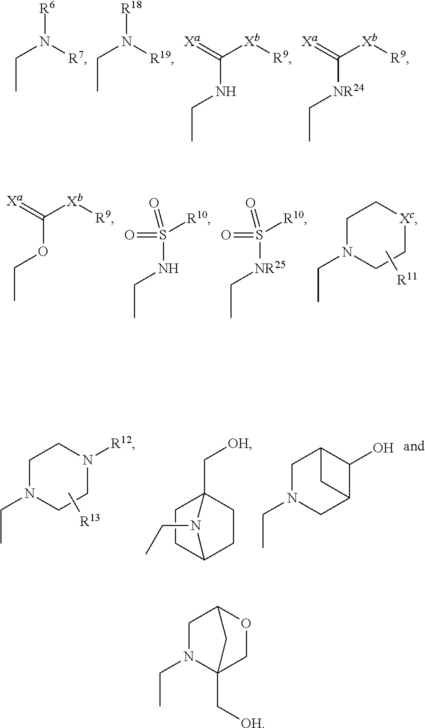

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—R⁵,

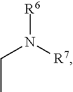

—(C₁-C₆ alkyl)-aryl,

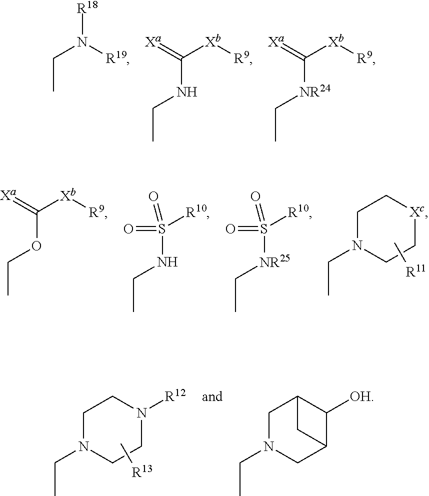

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—R⁵,

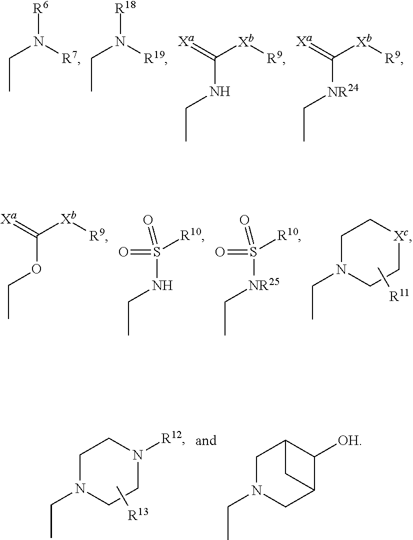

In some embodiments, in compounds of Formula (II), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (II), R⁶ and R⁷ are each independently selected from: —H, —C₁-C₆ alkyl, —C₃-C₈ cycloalkyl and —C(O)R¹⁷.

In some embodiments, in compounds of Formula (II), R⁶ is H, and R⁷ is selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (II), R⁶ is H, and R⁷ is selected from: —H, —C₁-C₆ alkyl, —C₃-C₈ cycloalkyl and —C(O)R¹⁷.

In some embodiments, in compounds of Formula (II), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (II), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (II), each R⁹ is independently selected from: —C₁-C₆ alkyl, —C₃-C₈ cycloalkyl, -aryl and —(C₁-C₆ alkyl)-aryl.

In some embodiments, in compounds of Formula (II), each R⁹ is independently selected from: —C₁-C₆ alkyl and —(C₁-C₆ alkyl)-aryl.

In some embodiments, in compounds of Formula (II), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (II), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (II), each R¹⁰ is independently selected from: —C₁-C₆ alkyl, —NR¹⁴R^{14′}, -aryl and —(C₁-C₆ alkyl)-aryl.

In some embodiments, in compounds of Formula (II), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (II), R^{10′} is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (II), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (II), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (II), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

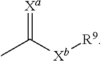

In some embodiments, in compounds of Formula (II), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (II), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (II), R¹⁶ is selected from: -aryl, -heteroaryl and —(C₁-C₆ alkyl)-aryl.

In some embodiments, in compounds of Formula (II), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (II), R¹⁷ is —C₁-C₆ alkyl.

In some embodiments, in compounds of Formula (II), R¹⁷ is selected from: unsubstituted C₁-C₆ alkyl, —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted aryl, -hydroxyaryl, -aminoaryl, -heteroaryl and —(C₁-C₆ alkyl)-aminoaryl.

In some embodiments, in compounds of Formula (II), R¹⁸ and R¹⁹ taken together with the N atom to which they are bonded form a 4-, 5-, 6- or 7-membered ring having 0 to 3 substituents selected from: halogen, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (II), X^{a} and X^{b} are each independently selected from: NH and O.

Combinations of any of the foregoing embodiments for compounds of Formula (II) are also contemplated and each combination forms a separate embodiment for the purposes of the present disclosure.

In certain embodiments, the compound of Formula (I) has Formula (III):

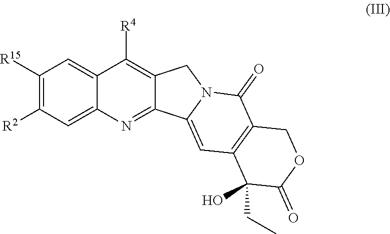

wherein:
R² is selected from: —H, —CH₃, —CF₃, —F, —Br, —Cl, —OH, —OCH₃ and —OCF₃;
R¹⁵ is selected from: —H, —CH₃, —CHF₂, —CF₃, —F, —Br, —Cl, —OH, —OCH₃ and —OCF₃;
R⁴ is selected from:

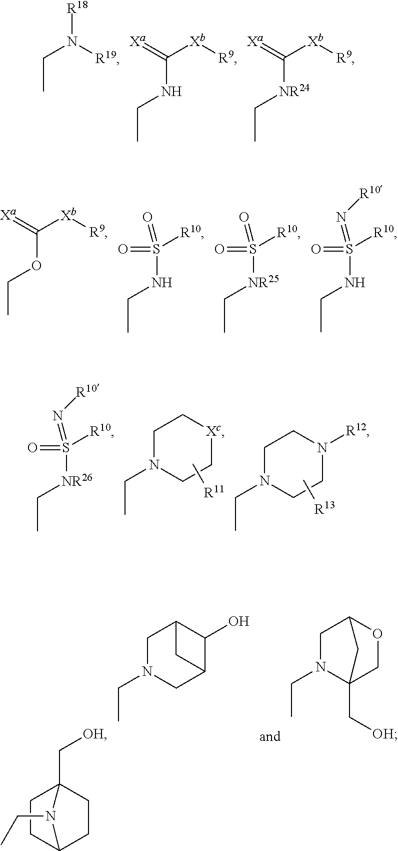

R⁵ is selected from: —H, —C₁-C₆ alkyl, —C₃-C₈ cycloalkyl, -aryl, -heteroaryl and —(C₁-C₆ alkyl)-aryl;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
R^{10′} is selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

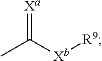

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from: O, S and S(O)₂.

In some embodiments, in compounds of Formula (III), R² is selected from: —H, —CH₃, —CF₃, —F, —Cl, —OCH₃ and —OCF₃.

In some embodiments, in compounds of Formula (III), R² is selected from: —H, —F and —Cl.

In some embodiments, in compounds of Formula (III), R¹⁵ is selected from: —CH₃, —CF₃, —OCH₃ and —OCF₃.

In some embodiments, in compounds of Formula (III), R¹⁵ is selected from: —CH₃ and —OCH₃.

In some embodiments, in compounds of Formula (III), R² is selected from: —H, —F and —Cl, and R¹⁵ is selected from: —CH₃, —CF₃, —OCH₃ and —OCF₃.

In some embodiments, in compounds of Formula (III), R² is selected from: —H, —F and —Cl, and R⁵ is selected from: —CH₃ and —OCH₃.

In some embodiments, in compounds of Formula (III), R⁴ is selected from:

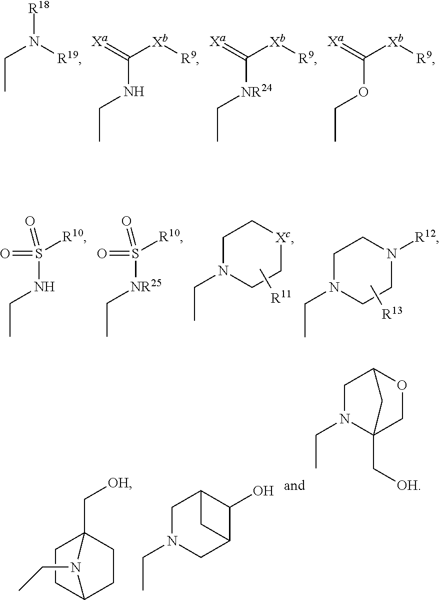

In some embodiments, in compounds of Formula (III), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (III), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (III), each R⁹ is independently selected from: —C₁-C₆ alkyl, —C₃-C₈ cycloalkyl, -aryl and —(C₁-C₆ alkyl)-aryl.

In some embodiments, in compounds of Formula (III), each R⁹ is independently selected from: —C₁-C₆ alkyl and —(C₁-C₆ alkyl)-aryl.

In some embodiments, in compounds of Formula (III), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (III), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (III), each R¹⁰ is independently selected from: —C₁-C₆ alkyl, —NR¹⁴R^{14′}, -aryl and —(C₁-C₆ alkyl)-aryl.

In some embodiments, in compounds of Formula (III), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (III), R^{10′} is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (III), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (III), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (III), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

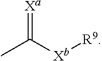

In some embodiments, in compounds of Formula (III), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (III), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (III), R¹⁶ is selected from: -aryl, -heteroaryl and —(C₁-C₆ alkyl)-aryl.

In some embodiments, in compounds of Formula (III), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (III), R¹⁸ and R¹⁹ taken together with the N atom to which they are bonded form a 4-, 5-, 6- or 7-membered ring having 0 to 3 substituents selected from: halogen, unsubstituted C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (III), X^{a} and X^{b} are each independently selected from: NH and O.

Combinations of any of the foregoing embodiments for compounds of Formula (III) are also contemplated and each combination forms a separate embodiment for the purposes of the present disclosure.

In certain embodiments, each alkyl, cycloalkyl, heterocycloalkyl, aryl and heteroaryl group as defined in any one of Formulae (I), (II) or (III) is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl. In some embodiments, each alkyl, cycloalkyl, heterocycloalkyl, aryl and heteroaryl group as defined in any one of Formulae (I), (II) or (III) is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

In certain embodiments, the camptothecin analogue comprised by the ADC according to the present disclosure is a compound having Formula (I) and is selected from the compounds shown in Tables 6 and 7.

In certain embodiments, the camptothecin analogue is a compound having Formula (II). In some embodiments, the camptothecin analogue is a compound having Formula (II), in which R² is F, and R²⁰ is H, —(C₁-C₆)—O—R⁵ or

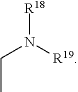

In some embodiments, the camptothecin analogue is a compound having Formula (II), in which R² is F; R²⁰ is H, —(C₁-C₆)—O—R⁵ or

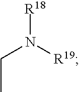

R⁵ is H, and R¹⁸ and R¹⁹ taken together with the N atom to which they are bonded form an unsubstituted 4-, 5-, 6-, or 7-membered ring. In some embodiments, the camptothecin analogue is a compound having Formula (II), in which R² is F; R²⁰ is —(C₁-C₆)—O—R⁵, and R⁵ is H. In certain embodiments, the camptothecin analogue is a compound having Formula (II) and is selected from the compounds shown in Table 6.

In certain embodiments, the camptothecin analogue is a compound having Formula (III). In certain embodiments, the camptothecin analogue is a compound having Formula (III), in which R² is F; R¹⁵ is —CH₃; R⁴ is

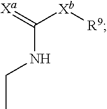

R⁹ is —C₁-C₆ hydroxyalkyl, and X^{a} and X^{b} are each O. In certain embodiments, the camptothecin analogue is a compound having Formula (III) and is selected from the compounds shown in Table 7.

In certain embodiments, the camptothecin analogue comprised by the ADC according to the present disclosure is Compound 139, Compound 140, Compound 141 or Compound 148. In some embodiments, the camptothecin analogue comprised by the ADC according to the present disclosure is Compound 139 or Compound 141.

TABLE 6
Exemplary Camptothecin Analogues of Formula (II)

| Structure | | |
|---|---|---|
| 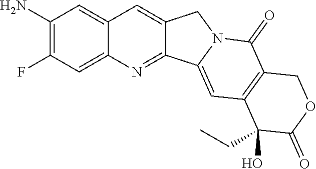 [SMILES: CC[C@@]1(O)C(=O)OCc2c1cc1n(c2=O)Cc2cc3cc(N)c(F)cc3nc2-1] | (S)-9-amino-4-ethyl-8-fluoro-4-hydroxy- 1,12-dihydro-14H- pyrano[3′,4′:6,7]indolizino[1,2- b]quinoline-3,14(4H)-dione | Compound 140 |
| 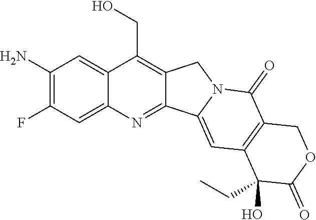 [SMILES: CC[C@@]1(O)C(=O)OCc2c1cc1n(c2=O)Cc2c-1nc1cc(F)c(N)cc1c2CO] | (S)-9-amino-4-ethyl-8-fluoro-4-hydroxy- 11-(hydroxymethyl)-1,12-dihydro-14H- pyrano[3′,4′:6,7]indolizino[1,2- b]quinoline-3,14(4H)-dione | Compound 141 |
| 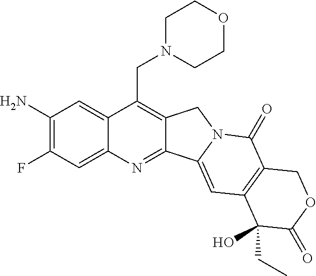 [SMILES: CC[C@@]1(O)C(=O)OCc2c1cc1n(c2=O)Cc2c-1nc1cc(F)c(N)cc1c2CN1CCOCC1] | (S)-9-amino-4-ethyl-8-fluoro-4-hydroxy- 11-(morpholinomethyl)-1,12-dihydro-14H- pyrano[3′,4′:6,7]indolizino[1,2- b]quinoline-3,14(4H)-dione | Compound 142 |
| 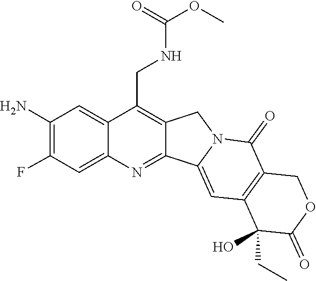 [SMILES: CC[C@@]1(O)C(=O)OCc2c1cc1n(c2=O)Cc2c-1nc1cc(F)c(N)cc1c2CNC(=O)OC] | methyl (S)-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)carbamate | Compound 143 |
| 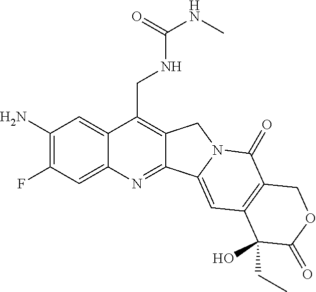 [SMILES: CC[C@@]1(O)C(=O)OCc2c1cc1n(c2=O)Cc2c-1nc1cc(F)c(N)cc1c2CNC(=O)NC] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-methylurea | Compound 144 |
| 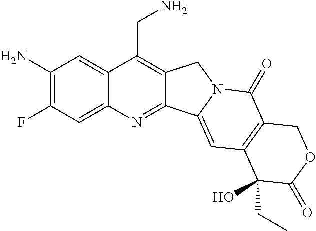 [SMILES: CC[C@@]1(O)C(=O)OCc2c1cc1n(c2=O)Cc2c-1nc1cc(F)c(N)cc1c2CN] | (S)-9-amino-11-(aminomethyl)-4-ethyl-8- fluoro-4-hydroxy-1,12-dihydro-14H- pyrano[3′,4′:6,7]indolizino[1,2- b]quinoline-3,14(4H)-dione | Compound 145 |
| 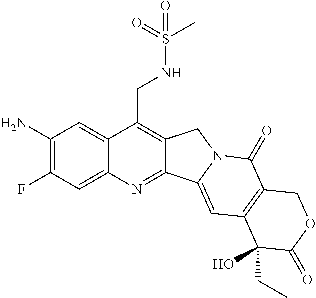 [SMILES: CC[C@@]1(O)C(=O)OCc2c1cc1n(c2=O)Cc2c-1nc1cc(F)c(N)cc1c2CNS(C)(=O)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11- yl)methyl)methanesulfonamide | Compound 146 |
| 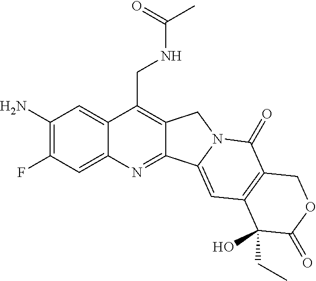 [SMILES: CC[C@@]1(O)C(=O)OCc2c1cc1n(c2=O)Cc2c-1nc1cc(F)c(N)cc1c2CNC(C)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)acetamide | Compound 147 |
| 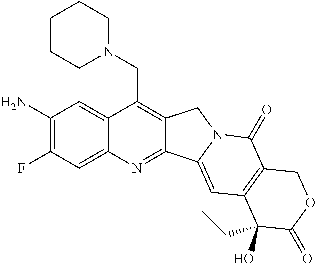 [SMILES: CC[C@@]1(O)C(=O)OCc2c1cc1n(c2=O)Cc2c-1nc1cc(F)c(N)cc1c2CN1CCCCC1] | (S)-9-amino-4-ethyl-8-fluoro-4-hydroxy- 11-(piperidin-1-ylmethyl)-1,12-dihydro- 14H-pyrano[3′,4′:6,7]indolizino[1,2- b]quinoline-3,14(4H)-dione | Compound 148 |
| 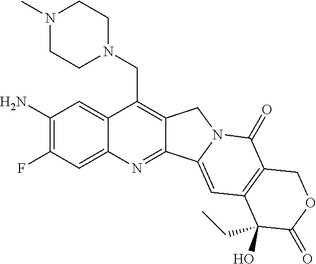 [SMILES: CC[C@@]1(O)C(=O)OCc2c1cc1n(c2=O)Cc2c-1nc1cc(F)c(N)cc1c2CN1CCN(C)CC1] | (S)-9-amino-4-ethyl-8-fluoro-4-hydroxy- 11-((4-methylpiperazin-1-yl)methyl)-1,12- dihydro-14H- pyrano[3′,4′:6,7]indolizino[1,2- b]quinoline-3,14(4H)-dione | Compound 149 |
| 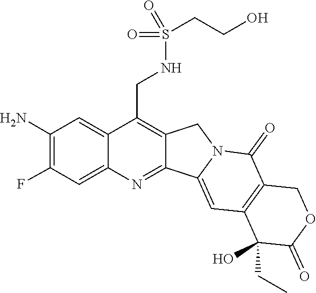 [SMILES: CC[C@@]1(O)C(=O)OCc2c1cc1n(c2=O)Cc2c-1nc1cc(F)c(N)cc1c2CNS(=O)(=O)CCO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 150 |
| 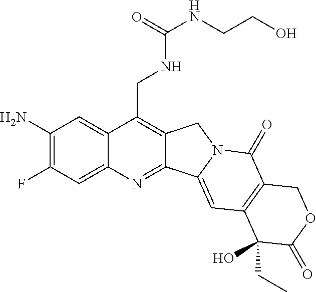 [SMILES: CC[C@@]1(O)C(=O)OCc2c1cc1n(c2=O)Cc2c-1nc1cc(F)c(N)cc1c2CNC(=O)NCCO] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 151 |
| 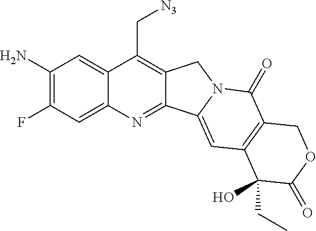 [SMILES: CC[C@@]1(O)C(=O)OCc2c1cc1n(c2=O)Cc2c-1nc1cc(F)c(N)cc1c2CN=[N+]=[N-]] | (S)-9-amino-11-(azidomethyl)-4-ethyl-8- fluoro-4-hydroxy-1,12-dihydro-14H- pyrano[3′,4′:6,7]indolizino[1,2- b]quinoline-3,14(4H)-dione | Compound 152 |
| 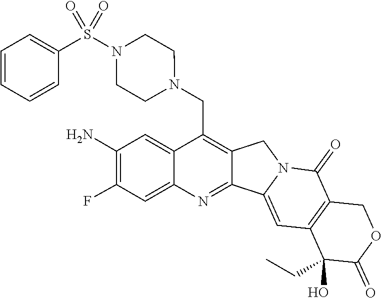 [SMILES: CC[C@@]1(O)C(=O)OCc2c1cc1n(c2=O)Cc2c-1nc1cc(F)c(N)cc1c2CN1CCN(S(=O)(=O)c2ccccc2)CC1] | (S)-9-amino-4-ethyl-8-fluoro-4-hydroxy- 11-((4-(phenylsulfonyl)piperazin-1- yl)methyl)-1,12-dihydro-14H- pyrano[3′,4′:6,7]indolizino[1,2- b]quinoline-3,14(4H)-dion | Compound 153 |
| 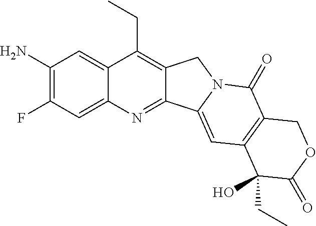 [SMILES: CCc1c2c(nc3cc(F)c(N)cc13)-c1cc3c(c(=O)n1C2)COC(=O)[C@]3(O)CC] | (S)-9-amino-4,11-diethyl-8-fluoro-4- hydroxy-1,12-dihydro-14H- pyrano[3′,4′:6,7]indolizino[1,2- b]quinoline-3,14(4H)-dione | Compound 154 |
| 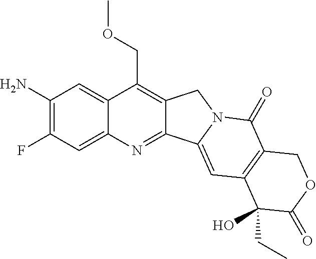 [SMILES: CC[C@@]1(O)C(=O)OCc2c1cc1n(c2=O)Cc2c-1nc1cc(F)c(N)cc1c2COC] | (S)-9-amino-4-ethyl-8-fluoro-4-hydroxy- 11-(methoxymethyl)-1,12-dihydro-14H- pyrano[3′,4′:6,7]indolizino[1,2- b]quinoline-3,14(4H)-dione | Compound 155 |
| 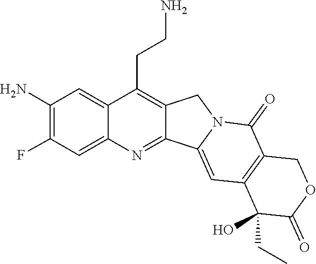 [SMILES: CC[C@@]1(O)C(=O)OCc2c1cc1n(c2=O)Cc2c-1nc1cc(F)c(N)cc1c2CCN] | (S)-9-amino-11-(2-aminoethyl)-4-ethyl-8- fluoro-4-hydroxy-1,12-dihydro-14H- pyrano[3′,4′:6,7]indolizino[1,2- b]quinoline-3,14(4H)-dione | Compound 156 |
| 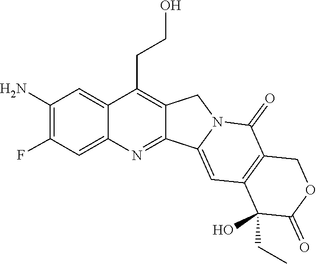 [SMILES: CC[C@@]1(O)C(=O)OCc2c1cc1n(c2=O)Cc2c-1nc1cc(F)c(N)cc1c2CCO] | (S)-9-amino-4-ethyl-8-fluoro-4-hydroxy- 11-(2-hydroxyethyl)-1,12-dihydro-14HI- pyrano[3′,4′:6,7]indolizino[1,2- b]quinoline-3,14(4H)-dione | Compound 157 |
| 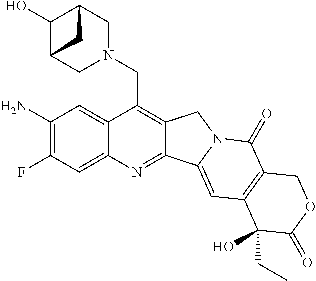 [SMILES: CC[C@@]1(O)C(=O)OCc2c1cc1n(c2=O)Cc2c-1nc1cc(F)c(N)cc1c2CN1C[C@H]2C[C@@H](C1)C2O] | (4S)-9-amino-4-ethyl-8-fluoro-4-hydroxy- 11-(((1R,5S)-6-hydroxy-3- azabicyclo[3.1.1]heptan-3-yl)methyl)-1,12- dihydro-14H- pyrano[3′,4′:6,7]indolizino[1,2- b]quinoline-3,14(4H)-dione | Compound 158 |
| 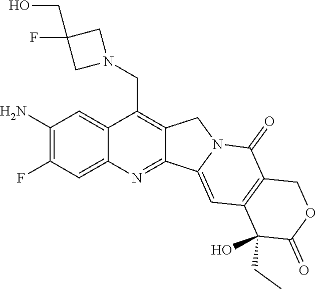 [SMILES: CC[C@@]1(O)C(=O)OCc2c1cc1n(c2=O)Cc2c-1nc1cc(F)c(N)cc1c2CN1CC(F)(CO)C1] | (S)-9-amino-4-ethyl-8-fluoro-11-((3- fluoro-3-(hydroxymethyl)azetidin-1- yl)methyl)-4-hydroxy-1,12-dihydro-14H- pyrano[3′,4′:6,7]indolizino[1,2- b]quinoline-3,14(4H)-dione | Compound 159 |
| 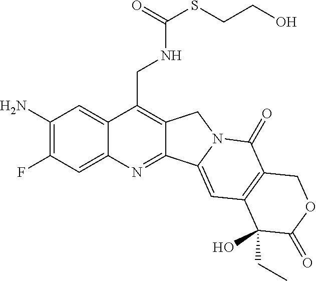 [SMILES: CC[C@@]1(O)C(=O)OCc2c1cc1n(c2=O)Cc2c-1nc1cc(F)c(N)cc1c2CNC(=O)SCCO] | S-(2-hydroxyethyl)(S)-((9-amino-4-ethyl- 8-fluoro-4-hydroxy-3,14-dioxo-3,4,12,14- tetrahydro-1H- pyrano[3′,4′:6,7]indolizino[1,2-b]quinolin- 11-yl)methyl)carbamothioate | Compound 160 |
| 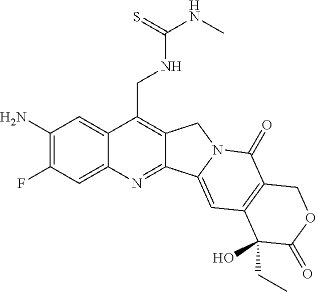 [SMILES: CC[C@@]1(O)C(=O)OCc2c1cc1n(c2=O)Cc2c-1nc1cc(F)c(N)cc1c2CNC(=S)NC] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-methylthiourea | Compound 161 |
| 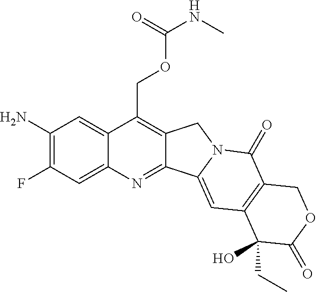 [SMILES: CC[C@@]1(O)C(=O)OCc2c1cc1n(c2=O)Cc2c-1nc1cc(F)c(N)cc1c2COC(=O)NC] | (S)-(9-amino-4-ethyl-8-fluoro-4-hydroxy- 3,14-dioxo-3,4,12,14-tetrahydro-1H- pyrano[3′,4′:6,7]indolizino[1,2-b]quinolin- 11-yl)methyl methylcarbamate | Compound 162 |
| 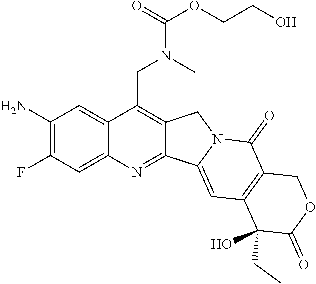 [SMILES: CC[C@@]1(O)C(=O)OCc2c1cc1n(c2=O)Cc2c-1nc1cc(F)c(N)cc1c2CN(C)C(=O)OCCO] | 2-hydroxyethyl (S)-((9-amino-4-ethyl-8- fluoro-4-hydroxy-3,14-dioxo-3,4,12,14- tetrahydro-1H- pyrano[3′,4′:6,7]indolizino[1,2-b]quinolin- 11-yl)methyl)(methyl)carbamate | Compound 163 |
| 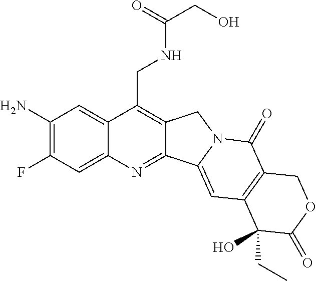 [SMILES: CC[C@@]1(O)C(=O)OCc2c1cc1n(c2=O)Cc2c-1nc1cc(F)c(N)cc1c2CNC(=O)CO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2- hydroxyacetamide | Compound 164 |
| 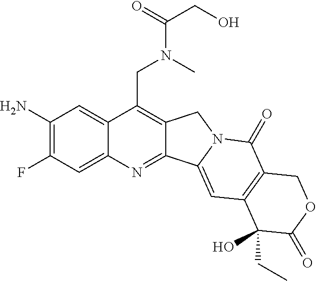 [SMILES: CC[C@@]1(O)C(=O)OCc2c1cc1n(c2=O)Cc2c-1nc1cc(F)c(N)cc1c2CN(C)C(=O)CO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2-hydroxy-N- methylacetamide | Compound 165 |
| 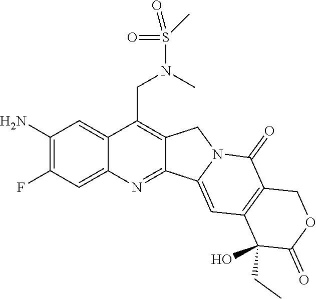 [SMILES: CC[C@@]1(O)C(=O)OCc2c1cc1n(c2=O)Cc2c-1nc1cc(F)c(N)cc1c2CN(C)S(C)(=O)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-N- methylmethanesulfonamide | Compound 166 |
| 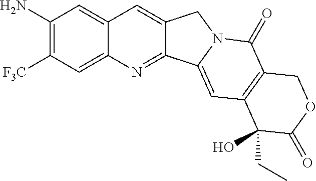 [SMILES: CC[C@@]1(O)C(=O)OCc2c1cc1n(c2=O)Cc2cc3cc(N)c(C(F)(F)F)cc3nc2-1] | (S)-9-amino-4-ethyl-4-hydroxy-8- (trifluoromethyl)-1,12-dihydro-14H- pyrano[3′,4′:6,7]indolizino[1,2- b]quinoline-3,14(4H)-dione | Compound 167 |
| 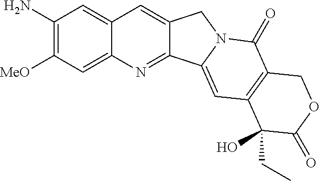 [SMILES: CC[C@@]1(O)C(=O)OCc2c1cc1n(c2=O)Cc2cc3cc(N)c(OC)cc3nc2-1] | (S)-9-amino-4-ethyl-4-hydroxy-8- methoxy-1,12-dihydro-14H- pyrano[3′,4′:6,7]indolizino[1,2- b]quinoline-3,14(4H)-dione | Compound 168 |
| 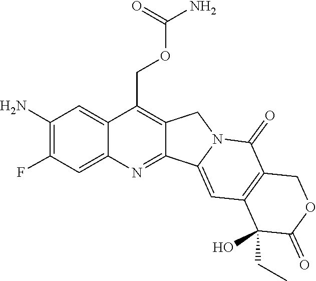 [SMILES: CC[C@@]1(O)C(=O)OCc2c1cc1n(c2=O)Cc2c-1nc1cc(F)c(N)cc1c2COC(N)=O] | (S)-(9-amino-4-ethyl-8-fluoro-4-hydroxy- 3,14-dioxo-3,4,12,14-tetrahydro-1H- pyrano[3′,4′:6,7]indolizino[1,2-b]quinolin- 11-yl)methyl carbamate | Compound 169 |
| 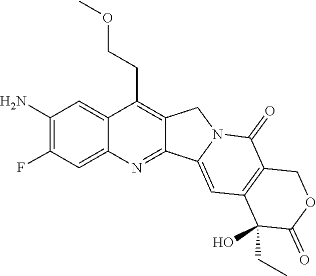 [SMILES: CC[C@@]1(O)C(=O)OCc2c1cc1n(c2=O)Cc2c-1nc1cc(F)c(N)cc1c2CCOC] | (S)-9-amino-4-ethyl-8-fluoro-4-hydroxy- 11-(2-methoxyethyl)-1,12-dihydro-14H- pyrano[3′,4′:6,7]indolizino[1,2- b]quinoline-3,14(4H)-dione | Compound 170 |
| 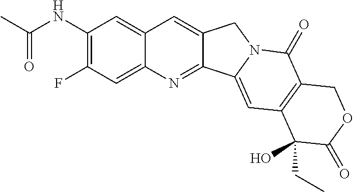 [SMILES: CC[C@@]1(O)C(=O)OCc2c1cc1n(c2=O)Cc2cc3cc(NC(C)=O)c(F)cc3nc2-1] | (S)-N-(4-ethyl-8-fluoro-4-hydroxy-3,14- dioxo-3,4,12,14-tetrahydro-1H- pyrano[3′,4′:6,7]indolizino[1,2-b]quinolin- 9-yl)acetamide | Compound 171 |

TABLE 7
Exemplary Camptothecin Analogues of Formula (III)

| Structure | | |
|---|---|---|
| 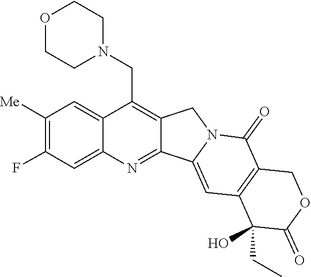 [SMILES: CC[C@@]1(O)C(=O)OCc2c1cc1n(c2=O)Cc2c-1nc1cc(F)c(C)cc1c2CN1CCOCC1] | (S)-4-ethyl-8-fluoro-4-hydroxy-9-methyl- 11-(morpholinomethyl)-1,12-dihydro-14H- pyrano[3′,4′:6,7]indolizino[1,2- b]quinoline-3,14(4H)-dione | Compound 100 |
| 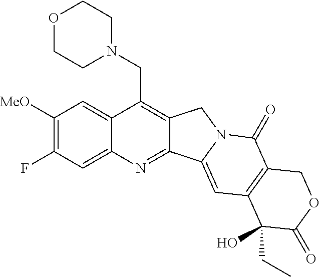 [SMILES: CC[C@@]1(O)C(=O)OCc2c1cc1n(c2=O)Cc2c-1nc1cc(F)c(OC)cc1c2CN1CCOCC1] | (S)-4-ethyl-8-fluoro-4-hydroxy-9-methoxy- 11-(morpholinomethyl)-1,12-dihydro-14H- pyrano[3′,4′:6,7]indolizino[1,2- b]quinoline-3,14(4H)-dione | Compound 101 |
| 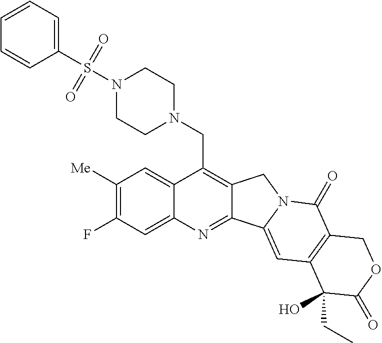 [SMILES: CC[C@@]1(O)C(=O)OCc2c1cc1n(c2=O)Cc2c-1nc1cc(F)c(C)cc1c2CN1CCN(S(=O)(=O)c2ccccc2)CC1] | (S)-4-ethyl-8-fluoro-4-hydroxy-9-methyl- 11-((4-(phenylsulfonyl)piperazin-1- yl)methyl)-1,12-dihydro-14H- pyrano[3′,4′:6,7]indolizino[1,2- b]quinoline-3,14(4H)-dione | Compound 102 |
| 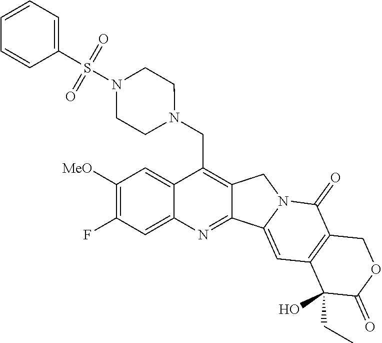 [SMILES: CC[C@@]1(O)C(=O)OCc2c1cc1n(c2=O)Cc2c-1nc1cc(F)c(OC)cc1c2CN1CCN(S(=O)(=O)c2ccccc2)CC1] | (S)-4-ethyl-8-fluoro-4-hydroxy-9-methoxy- 11-((4-(phenylsulfonyl)piperazin-1- yl)methyl)-1,12-dihydro-14H- pyrano[3′,4′:6,7]indolizino[1,2- b]quinoline-3,14(4H)-dione | Compound 103 |
| 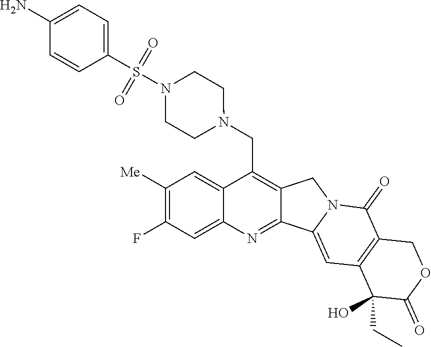 [SMILES: CC[C@@]1(O)C(=O)OCc2c1cc1n(c2=O)Cc2c-1nc1cc(F)c(C)cc1c2CN1CCN(S(=O)(=O)c2ccc(N)cc2)CC1] | (S)-11-((4-((4- aminophenyl)sulfony1)piperazin-1- yl)methyl)-4-ethyl-8-fluoro-4-hydroxy-9- methyl-1,12-dihydro-14//- pyrano[3′,4′:6,7]indolizino[1,2- b]quinoline-3,14(4H)-dione | Compound 104 |
| 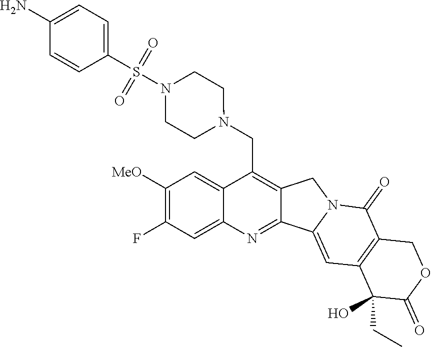 [SMILES: CC[C@@]1(O)C(=O)OCc2c1cc1n(c2=O)Cc2c-1nc1cc(F)c(OC)cc1c2CN1CCN(S(=O)(=O)c2ccc(N)cc2)CC1] | (S)-11-((4-((4- aminophenyl)sulfonyl)piperazin-1- yl)methyl)-4-ethyl-8-fluoro-4-hydroxy-9- methoxy-1,12-dihydro-14H- pyrano[3′,4′:6,7]indolizino[1,2- b]quinoline-3,14(4H)-dione | Compound 105 |
| 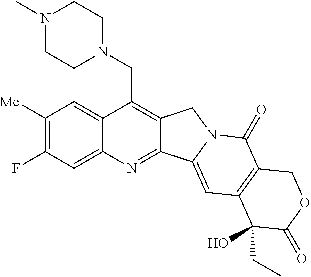 [SMILES: CC[C@@]1(O)C(=O)OCc2c1cc1n(c2=O)Cc2c-1nc1cc(F)c(C)cc1c2CN1CCN(C)CC1] | (S)-4-ethyl-8-fluoro-4-hydroxy-9-methyl- 11-((4-methylpiperazin-1-yl)methyl)-1,12- dihydro-14H- pyrano[3′,4′:6,7]indolizino[1,2- b]quinoline-3,14(4H)-dione | Compound 106 |
| 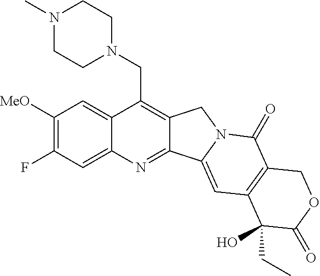 [SMILES: CC[C@@]1(O)C(=O)OCc2c1cc1n(c2=O)Cc2c-1nc1cc(F)c(OC)cc1c2CN1CCN(C)CC1] | (S)-4-ethyl-8-fluoro-4-hydroxy-9-methoxy- 11-((4-methylpiperazin-1-yl)methyl)-1,12- dihydro-14H- pyrano[3′,4′:6,7]indolizino[1,2- b]quinoline-3,14(4H)-dione | Compound 107 |
| 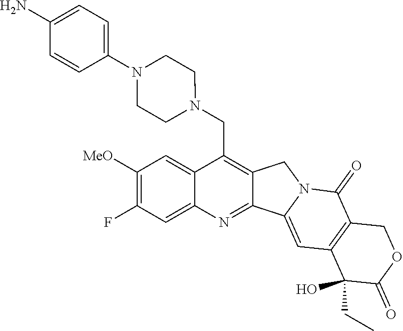 [SMILES: CC[C@@]1(O)C(=O)OCc2c1cc1n(c2=O)Cc2c-1nc1cc(F)c(OC)cc1c2CN1CCN(c2ccc(N)cc2)CC1] | (S)-11-((4-(4-aminophenyl)piperazin-1- yl)methyl)-4-ethyl-8-fluoro-4-hydroxy-9- methyl-1,12-dihydro-14H- pyrano[3′,4′:6,7]indolizino[1,2- b]quinoline-3,14(4H)-dione | Compound 108 |
| 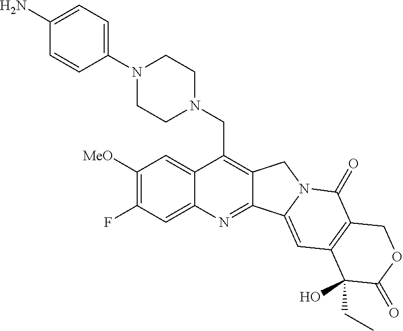 [SMILES: CC[C@@]1(O)C(=O)OCc2c1cc1n(c2=O)Cc2c-1nc1cc(F)c(OC)cc1c2CN1CCN(c2ccc(N)cc2)CC1] | (S)-11-((4-(4-aminophenyl)piperazin-1- yl)methyl)-4-ethyl-8-fluoro-4-hydroxy-9- methoxy-1,12-dihydro-14H- pyrano[3′,4′:6,7]indolizino[1,2- b]quinoline-3,14(4H)-dione | Compound 109 |
| 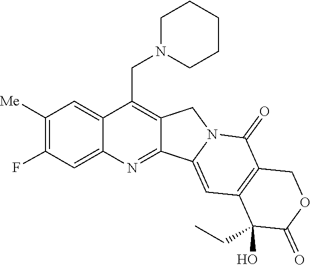 [SMILES: CC[C@@]1(O)C(=O)OCc2c1cc1n(c2=O)Cc2c-1nc1cc(F)c(C)cc1c2CN1CCCCC1] | (S)-4-ethyl-8-fluoro-4-hydroxy-9-methyl- 11-(piperidin-1-ylmethyl)-1,12-dihydro- 14H-pyrano[3′,4′:6,7]indolizino[1,2- b]quinoline-3,14(4H)-dione | Compound 110 |
| 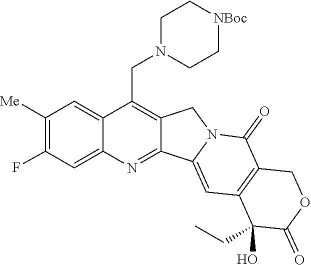 [SMILES: CC[C@@]1(O)C(=O)OCc2c1cc1n(c2=O)Cc2c-1nc1cc(F)c(C)cc1c2CN1CCN(C(=O)OC(C)(C)C)CC1] | tert-butyl (S)-4-((4-ethyl-8-fluoro-4- hydroxy-9-methyl-3,14-dioxo-3,4,12,14- tetrahydro-1/- pyrano[3′,4′:6,7]indolizino[1,2-b]quinolin- 11-yl)methyl)piperazine-1-carboxylate | Compound 111 |
| 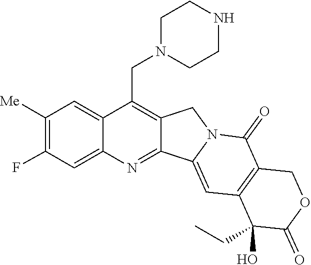 [SMILES: CC[C@@]1(O)C(=O)OCc2c1cc1n(c2=O)Cc2c-1nc1cc(F)c(C)cc1c2CN1CCNCC1] | (S)-4-ethyl-8-fluoro-4-hydroxy-9-methyl- 11-(piperazin-1-ylmethyl)-1,12-dihydro- 14H-pyrano[3′,4′:6,7]indolizino[1,2- b]quinoline-3,14(4H)-dione | Compound 112 |
| 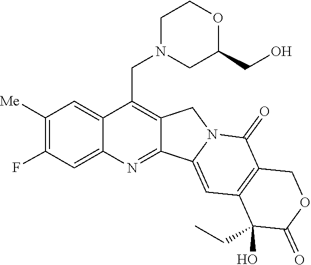 [SMILES: CC[C@@]1(O)C(=O)OCc2c1cc1n(c2=O)Cc2c-1nc1cc(F)c(C)cc1c2CN1CCO[C@@H](CO)C1] | (S)-4-ethyl-8-fluoro-4-hydroxy-11-(((R)-2- (hydroxymethyl)morpholino)methyl)-9- methyl-1,12-dihydro-14H- pyrano[3′,4′:6,7]indolizino[1,2- b]quinoline-3,14(4H)-dione | Compound 113 |
| 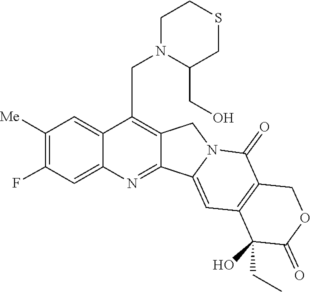 [SMILES: CC[C@@]1(O)C(=O)OCc2c1cc1n(c2=O)Cc2c-1nc1cc(F)c(C)cc1c2CN1CCSCC1CO] | (4S)-4-ethyl-8-fluoro-4-hydroxy-11-((3- (hydroxymethyl)thiomorpholino)methyl)- 9-methyl-1,12-dihydro-14H- pyrano[3′,4′:6,7]indolizino[1,2- b]quinoline-3,14(4H)-dione | Compound 114 |
| 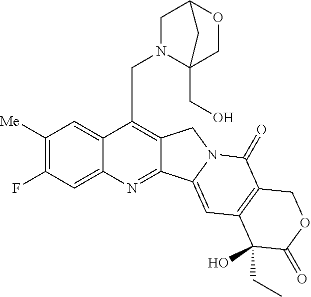 [SMILES: CC[C@@]1(O)C(=O)OCc2c1cc1n(c2=O)Cc2c-1nc1cc(F)c(C)cc1c2CN1CC2CC1(CO)CO2] | (4S)-4-ethyl-8-fluoro-4-hydroxy-11-((4- (hydroxymethyl)-2-oxa-5- azabicyclo[2.2.1]heptan-5-yl)methyl)-9- methyl-1,12-dihydro-14H- pyrano[3′,4′:6,7]indolizino[1,2- b]quinoline-3,14(4H)-dione | Compound 115 |
| 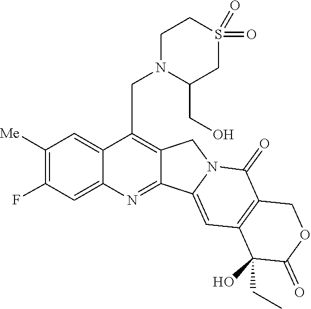 [SMILES: CC[C@@]1(O)C(=O)OCc2c1cc1n(c2=O)Cc2c-1nc1cc(F)c(C)cc1c2CN1CCS(=O)(=O)CC1CO] | (4S)-4-ethyl-8-fluoro-4-hydroxy-11-((3- (hydroxymethyl)-1,1- dioxidothiomorpholino)methyl)-9-methyl- 1,12-dihydro-14/I- pyrano[3′,4′:6,7]indolizino[1,2- b]quinoline-3,14(4H)-dione | Compound 116 |
| 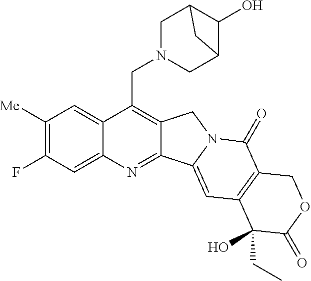 [SMILES: CC[C@@]1(O)C(=O)OCc2c1cc1n(c2=O)Cc2c-1nc1cc(F)c(C)cc1c2CN1CC2CC(C1)C2O] | (4S)-4-ethyl-8-fluoro-4-hydroxy-11-((6- hydroxy-3-azabicyclo[3.1.1]heptan-3- yl)methyl)-9-methyl-1,12-dihydro-14H- pyrano[3′,4′:6,7]indolizino[1,2- b]quinoline-3,14(4H)-dione | Compound 117 |
| 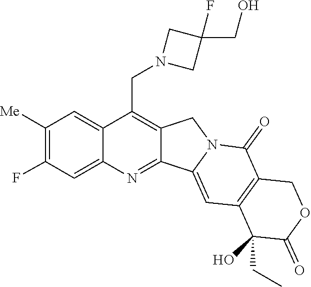 [SMILES: CC[C@@]1(O)C(=O)OCc2c1cc1n(c2=O)Cc2c-1nc1cc(F)c(C)cc1c2CN1CC(F)(CO)C1] | (S)-4-ethyl-8-fluoro-11-((3-fluoro-3- (hydroxymethyl)azetidin-1-yl)methyl)-4- hydroxy-9-methyl-1,12-dihydro-14H- pyrano[3′,4′:6,7]indolizino[1,2- b]quinoline-3,14(4H)-dione | Compound 118 |
| 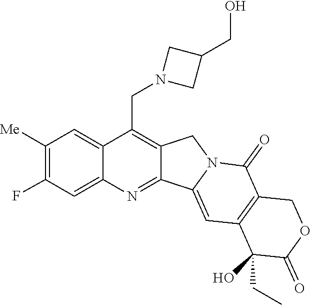 [SMILES: CC[C@@]1(O)C(=O)OCc2c1cc1n(c2=O)Cc2c-1nc1cc(F)c(C)cc1c2CN1CC(CO)C1] | (S)-4-ethyl-8-fluoro-4-hydroxy-11-((3- (hydroxymethyl)azetidin-1-yl)methyl)-9- methyl-1,12-dihydro-14H- pyrano[3′,4′:6,7]indolizino[1,2- b]quinoline-3,14(4H)-dione | Compound 119 |
| 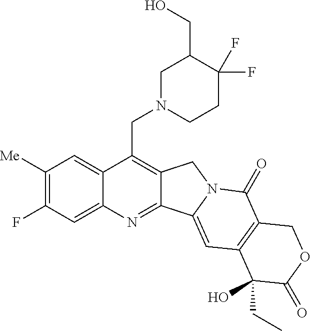 [SMILES: CC[C@@]1(O)C(=O)OCc2c1cc1n(c2=O)Cc2c-1nc1cc(F)c(C)cc1c2CN1CCC(F)(F)C(CO)C1] | (4S)-11-((4,4-difluoro-3- (hydroxymethyl)piperidin-1-yl)methyl)-4- ethyl-8-fluoro-4-hydroxy-9-methyl-1,12- dihydro-14H- pyrano[3′,4′:6,7]indolizino[1,2- b]quinoline-3,14(4H)-dione | Compound 120 |
| 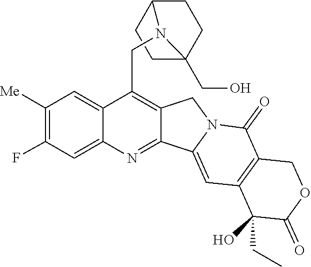 [SMILES: CC[C@@]1(O)C(=O)OCc2c1cc1n(c2=O)Cc2c-1nc1cc(F)c(C)cc1c2CN1C2CCC1(CO)CC2] | (4S)-11-((4,4-difluoro-3- (hydroxymethyl)piperidin-1-yl)methyl)-4- ethyl-8-fluoro-4-hydroxy-9-methyl-1,12- dihydro-14H- pyrano[3′,4′:6, 7]indolizino[1,2- b]quinoline-3,14(4H)-dione | Compound 121 |
| 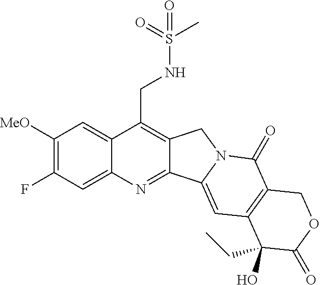 [SMILES: CC[C@@]1(O)C(=O)OCc2c1cc1n(c2=O)Cc2c-1nc1cc(F)c(OC)cc1c2CNS(C)(=O)=O] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11- yl)methyl)methanesulfonamide | Compound 122 |
| 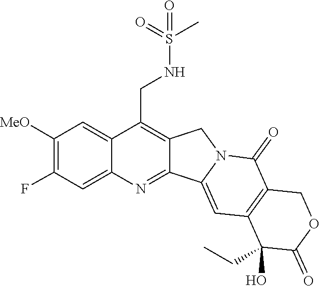 [SMILES: CC[C@@]1(O)C(=O)OCc2c1cc1n(c2=O)Cc2c-1nc1cc(F)c(OC)cc1c2CNS(C)(=O)=O] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11- yl)methyl)methanesulfonamide | Compound 123 |
| 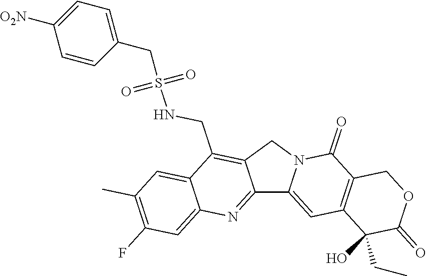 [SMILES: CC[C@@]1(O)C(=O)OCc2c1cc1n(c2=O)Cc2c-1nc1cc(F)c(C)cc1c2CNS(=O)(=O)Cc1ccc([N+](=O)[O-])cc1] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-1-(4- nitrophenyl)methanesulfonamide | Compound 124 |
| 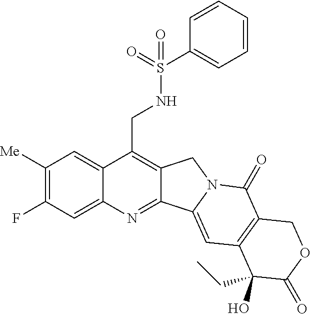 [SMILES: CC[C@@]1(O)C(=O)OCc2c1cc1n(c2=O)Cc2c-1nc1cc(F)c(C)cc1c2CNS(=O)(=O)c1ccccc1] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11- yl)methyl)benzenesulfonamide | Compound 125 |
| 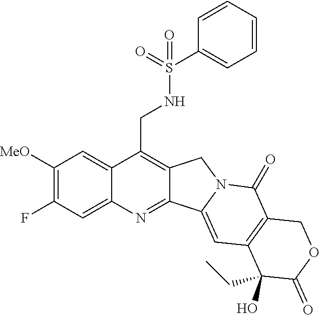 [SMILES: CC[C@@]1(O)C(=O)OCc2c1cc1n(c2=O)Cc2c-1nc1cc(F)c(OC)cc1c2CNS(=O)(=O)c1ccccc1] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11- yl)methyl)benzenesulfonamide | Compound 126 |
| 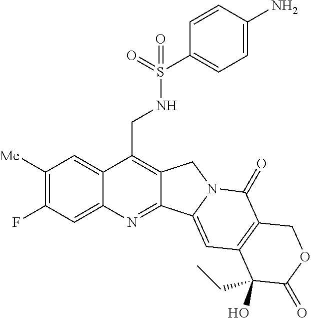 [SMILES: CC[C@@]1(O)C(=O)OCc2c1cc1n(c2=O)Cc2c-1nc1cc(F)c(C)cc1c2CNS(=O)(=O)c1ccc(N)cc1] | (S)-4-amino-N-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)benzenesulfonamide | Compound 127 |
| 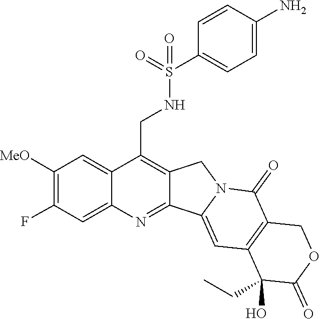 [SMILES: CC[C@@]1(O)C(=O)OCc2c1cc1n(c2=O)Cc2c-1nc1cc(F)c(OC)cc1c2CNS(=O)(=O)c1ccc(N)cc1] | (S)-4-amino-N-((4-ethyl-8-fluoro-4- hydroxy-9-methoxy-3,14-dioxo-3,4,12,14- tetrahydro-1H- pyrano[3′,4′:6,7]indolizino[1,2-b]quinolin- 11-yl)methyl)benzenesulfonamide | Compound 128 |
| 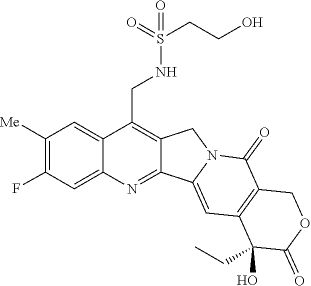 [SMILES: CC[C@@]1(O)C(=O)OCc2c1cc1n(c2=O)Cc2c-1nc1cc(F)c(C)cc1c2CNS(=O)(=O)CCO] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 129 |
| 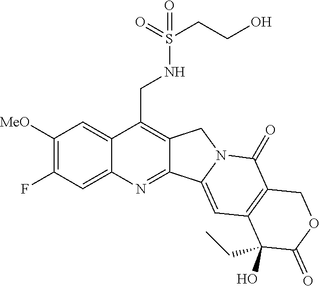 [SMILES: CC[C@@]1(O)C(=O)OCc2c1cc1n(c2=O)Cc2c-1nc1cc(F)c(OC)cc1c2CNS(=O)(=O)CCO] | (S)-N-((4-ethyl-8-fluoro-4-hydroxy-9- methoxy-3,14-dioxo-3,4, 12, 14-tetrahydro- 1H-pyrano[3′,4′:6,7]indolizino[1,2- b]quinolin-11-yl)methyl)-2- hydroxyethane-1-sulfonamide | Compound 130 |
| 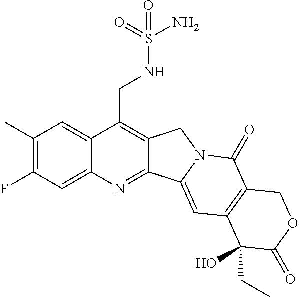 [SMILES: CC[C@@]1(O)C(=O)OCc2c1cc1n(c2=O)Cc2c-1nc1cc(F)c(C)cc1c2CNS(N)(=O)=O] | (S)-((4-ethyl-8-fluoro-4-hydroxy-9-methyl- 3,14-dioxo-3,4,12,14-tetrahydro-1H- pyrano[3′,4′:6,7]indolizino[1,2-b]quinolin- 11-yl)methyl)sulfamide | Compound 131 |
| 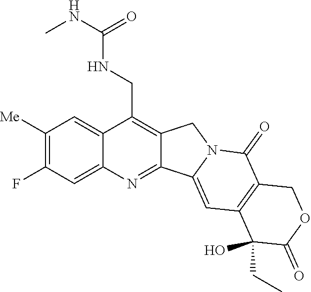 [SMILES: CC[C@@]1(O)C(=O)OCc2c1cc1n(c2=O)Cc2c-1nc1cc(F)c(C)cc1c2CNC(=O)NC] | (S)-1-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-3-methylurea | Compound 132 |
| 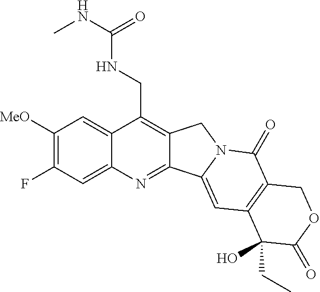 [SMILES: CC[C@@]1(O)C(=O)OCc2c1cc1n(c2=O)Cc2c-1nc1cc(F)c(OC)cc1c2CNC(=O)NC] | (S)-1-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-3-methylurea | Compound 133 |
| 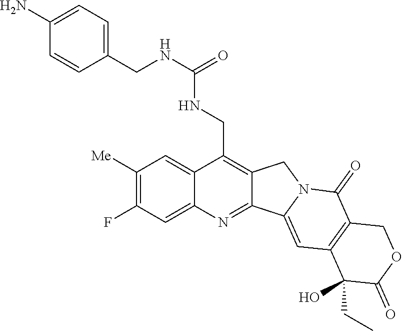 [SMILES: CC[C@@]1(O)C(=O)OCc2c1cc1n(c2=O)Cc2c-1nc1cc(F)c(C)cc1c2CNC(=O)NCc1ccc(N)cc1] | (S)-1-(4-aminobenzyl)-3-((4-ethyl-8- fluoro-4-hydroxy-9-methyl-3,14-dioxo- 3,4,12,14-tetrahydro-1H- pyrano[3′,4′:6,7]indolizino[1,2-b]quinolin- 11-yl)methyl)urea | Compound 134 |
| 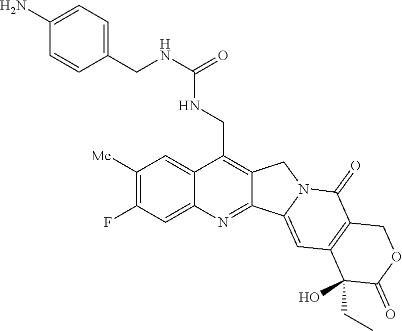 [SMILES: CC[C@@]1(O)C(=O)OCc2c1cc1n(c2=O)Cc2c-1nc1cc(F)c(C)cc1c2CNC(=O)NCc1ccc(N)cc1] | (S)-1-(4-aminobenzyl)-3-((4-ethyl-8- fluoro-4-hydroxy-9-methoxy-3,14-dioxo- 3,4,12,14-tetrahydro-1/- pyrano[3′,4′:6,7]indolizino[1,2-b]quinolin- 11-yl)methyl)urea | Compound 135 |
| 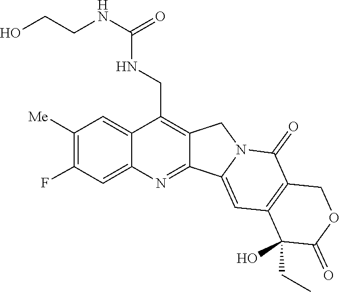 [SMILES: CC[C@@]1(O)C(=O)OCc2c1cc1n(c2=O)Cc2c-1nc1cc(F)c(C)cc1c2CNC(=O)NCCO] | (S)-1-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 136 |
| 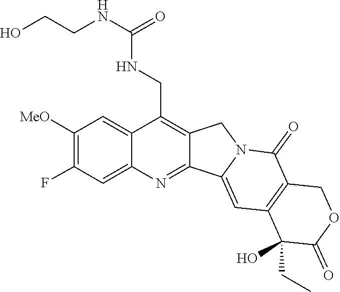 [SMILES: CC[C@@]1(O)C(=O)OCc2c1cc1n(c2=O)Cc2c-1nc1cc(F)c(OC)cc1c2CNC(=O)NCCO] | (S)-1-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 137 |
| 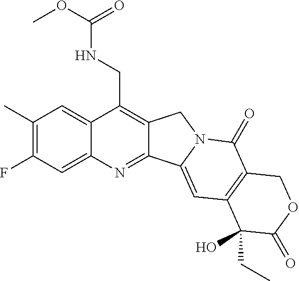 [SMILES: CC[C@@]1(O)C(=O)OCc2c1cc1n(c2=O)Cc2c-1nc1cc(F)c(C)cc1c2CNC(=O)OC] | methyl (S)-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)carbamate | Compound 138 |
| 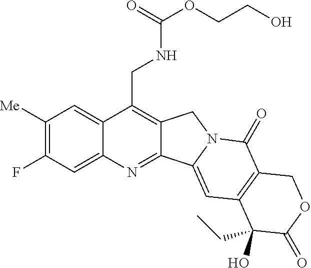 [SMILES: CC[C@@]1(O)C(=O)OCc2c1cc1n(c2=O)Cc2c-1nc1cc(F)c(C)cc1c2CNC(=O)OCCO] | 2-hydroxyethyl (S)-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)carbamate | Compound 139 |

It is to be understood that reference to compounds of Formula (I) throughout this disclosure, includes in various embodiments, compounds of Formula (II) and Formula (III), as well as the individual compounds shown in Tables 6 and 7, to the same extent as if embodiments reciting each of these Formulae or compounds individually were specifically recited.

### Antibody-Drug Conjugates

The present disclosure relates to antibody-drug conjugates (ADCs) comprising an anti-NaPi2b antibody construct conjugated to a camptothecin analogue having Formula (I). In certain embodiments, the ADC has Formula (X):

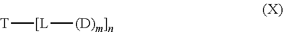

wherein:
T is an anti-NaPi2b antibody construct as described herein;
L is a linker;
D is a camptothecin analogue having Formula (I);
m is an integer between 1 and 4, and
n is an integer between 1 and 10.

In certain embodiments, in conjugates of Formula (X), m is between 1 and 2. In some embodiments, m is 1.

In some embodiments, in conjugates of Formula (X), n is between 1 and 8, for example, between 2 and 8. In some embodiments, n is between 4 and 8.

In certain embodiments, in conjugates of Formula (X), m is between 1 and 2, and n is between 2 and 8, or between 4 and 8. In some embodiments, in conjugates of Formula (X), m is 1, and n is between 2 and 8, or between 4 and 8. In other embodiments, in conjugates of Formula (X), m is between 1 and 2, and n is between 2 and 6. In still other embodiments, in conjugates of Formula (X), m is 1, and n is between 2 and 6. In yet other embodiments, in conjugates of Formula (X), m is 1, and n is between 3 and 5. In additional embodiments, in conjugates of Formula (X), m is 1, and n is about 4.

As noted above and reflected by parameters m and n in Formula (X), the anti-NaPi2b antibody construct, “T,” can be conjugated to more than one compound of Formula (I), “D.” Those skilled in the art will appreciate that, while any particular anti-NaPi2b antibody construct T is conjugated to an integer number of compounds D, analysis of a preparation of the conjugate to determine the ratio of compound D to anti-NaPi2b antibody construct T may give a non-integer result, reflecting a statistical average. This ratio of compound D to targeting moiety T may generally be referred to as the drug-to-antibody ratio, or “DAR.” Accordingly, conjugate preparations having non-integer DARs are intended to be encompassed by Formula (X). In one embodiment, ADCs of the disclosure have a DAR of between 2 and 8. In other embodiments, the ADCs of the disclosure have a DAR of between 3 and 5. In yet other embodiments, the ADCs of the disclosure have a DAR between 3.5 and 4.5.

In certain embodiments, in the conjugates of Formula (X), D is a compound of Formula (II) or Formula (III). In certain embodiments, in the conjugates of Formula (X), D is a compound selected from the compounds shown in Tables 6 and 7. In certain embodiments, in the conjugates of Formula (X), D is Compound 139, Compound 140, Compound 141 or Compound 148. In some embodiments, in the conjugates of Formula (X), D is Compound 139 or Compound 141.

Certain embodiments of the present disclosure relate to ADCs having Formula (X), in which D is a compound of Formula (IV):

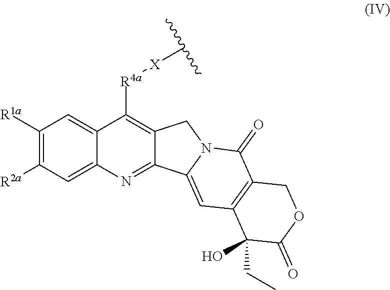

wherein:
R^{1a} is selected from: —H, —CH₃, —CHF₂, —CF₃, —F, —Br, —Cl, —OH, —OCH₃, —OCF₃ and —NH₂;
R^{2a} is selected from: —H, —CH₃, —CF₃, —F, —Br, —Cl, —OH, —OCH₃ and —OCF₃;
X is —O—, —S— or —NH—, and R^{4a} is selected from:

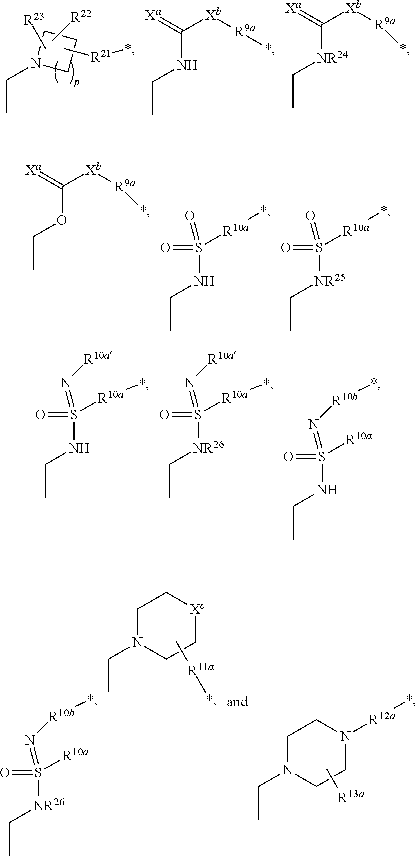

wherein * is the point of attachment to X, and wherein p is 1, 2, 3 or 4; or
X is O, and R^{4a}—X— is selected from

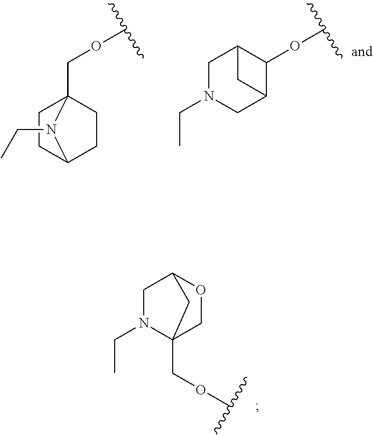 [SMILES: *OCC12CCC(CC1)N2CC ; *OC1C2CC1CN(CC)C2 ; *OCC12COC(CN1CC)C2]

R^{5a} is selected from: —C₁-C₆ alkyl, —C₃-C₈ cycloalkyl, -aryl, -heteroaryl and —(C₁-C₆ alkyl)-aryl;
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}=X;
each R^{10a} is independently selected from: —C₁-C₆ alkyl, —C₃-C₈ cycloalkyl, -aryl, -heteroaryl, —(C₁-C₆ alkyl)-aryl and

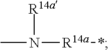

each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, —S(O)₂R^{16a} and

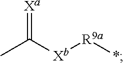

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{1a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

In some embodiments, in compounds of Formula (IV), R^{1a} is selected from: —CH₃, —CF₃, —OCH₃, —OCF₃ and —NH₂.

In some embodiments, in compounds of Formula (IV), R^{1a} is selected from: —CH₃, —CF₃, —OCH₃ and —OCF₃.

In some embodiments, in compounds of Formula (IV), R^{1a} is selected from: —CH₃, —OCH₃ and NH₂.

In some embodiments, in compounds of Formula (IV), R^{1a} is selected from: —CH₃ and —OCH₃.

In some embodiments, in compounds of Formula (IV), R^{2a} is selected from: —H, —CH₃, —CF₃, —F, —Cl, —OCH₃ and —OCF₃.

In some embodiments, in compounds of Formula (IV), R^{2a} is selected from: —H, —F and —Cl.

In some embodiments, in compounds of Formula (IV), R^{2a} is —F.

In some embodiments, in compounds of Formula (IV), X is —O—, —S— or —NH—, and R^{4a} is selected from:

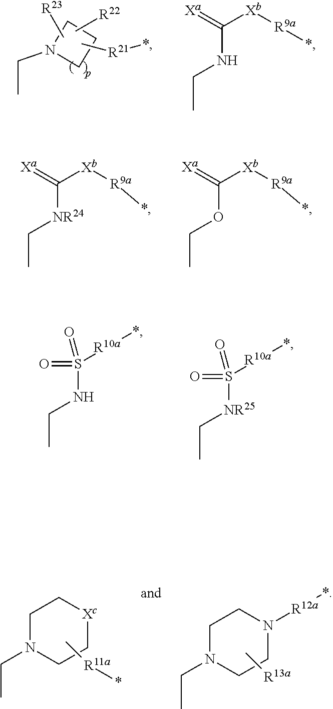

In some embodiments, in compounds of Formula (IV), X is —O— or —NH—.

In some embodiments, in compounds of Formula (IV), each R^{9a} is independently selected from: —C₁-C₆ alkyl, —C₃-C₈ cycloalkyl, -aryl and —(C₁-C₆ alkyl)-aryl.

In some embodiments, in compounds of Formula (IV), each R^{9a} is independently selected from: —C₁-C₆ alkyl and —(C₁-C₆ alkyl)-aryl.

In some embodiments, in compounds of Formula (IV), each R^{10a} is independently selected from: —C₁-C₆ alkyl, —C₃-C₈ cycloalkyl, -aryl, —(C₁-C₆ alkyl)-aryl and

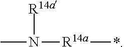

In some embodiments, in compounds of Formula (IV), each R^{10a} is independently selected from: —C₁-C₆ alkyl, -aryl, —(C₁-C₆ alkyl)-aryl and

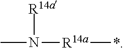

In some embodiments, in compounds of Formula (IV), R^{12a} is selected from: —C₁-C₆ alkyl, -aryl, —(C₁-C₆ alkyl)-aryl and —S(O)₂R¹⁶.

In some embodiments, in compounds of Formula (IV), R^{13a} is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (IV), R^{14a′} is selected from: H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (IV), R^{16a} is selected from: -aryl, -heteroaryl and —(C₁-C₆ alkyl)-aryl.

In some embodiments, in compounds of Formula (IV), R²² and R²³ are each independently selected from: —H, -halogen, unsubstituted —C₁-C₆ alkyl, —C₁-C₆ haloalkyl, —C₁-C₆ aminoalkyl, —C₁-C₆ hydroxyalkyl and —C₃-C₈ cycloalkyl.

In some embodiments, in compounds of Formula (IV), X^{a} and X^{b} are each independently selected from: NH and O.

In some embodiments, in compounds of Formula (IV), X^{a} and X^{b} are each O.

In some embodiments, in compounds of Formula (IV), X is O; R^{4a} is

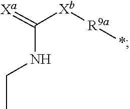

X^{a} and X^{b} are each O, and R^{9a} is —C₁-C₆ alkyl.

In some embodiments, in compounds of Formula (IV), R^{1a} is —CH₃ or —OCH₃; X is O; R^{4a} is

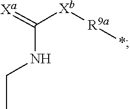

X^{a} and X^{b} are each O; and R^{9a} is —C₁-C₆ alkyl.

In some embodiments, in compounds of Formula (IV), R^{1a} is —CH₃ or —OCH₃; R^{2a} is H or F; X is O; R^{4a} is

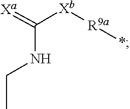

X^{a} and X^{b} are each O; and R^{9a} is —C₁-C₆ alkyl.

Other combinations of any of the foregoing embodiments for compounds of Formula (IV) are also contemplated and each combination forms a separate embodiment for the purposes of the present disclosure.

Certain embodiments of the present disclosure relate to ADCs having Formula (X), in which D is a compound of Formula (V):

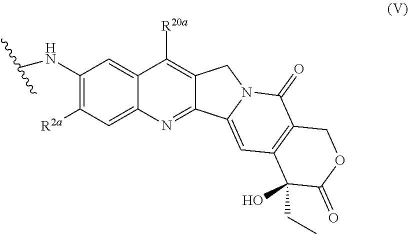

wherein:
R^{2a} is selected from: —CH₃, —CF₃, —F, —Br, —Cl, —OH, —OCH₃ and —OCF₃;
R^{20a} is selected from: —H, —C₁-C₆ alkyl, —C₃-C₈ cycloalkyl, —(C₁-C₆ alkyl)-O—R⁵,

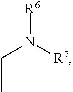

—CO₂R⁸, -aryl, -heteroaryl, —(C₁-C₆ alkyl)-aryl,

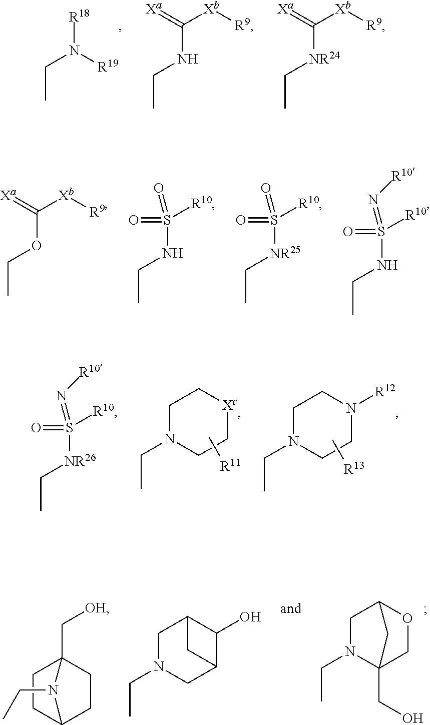

R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, -aryl, -heteroaryl, —(C₁-C₆ alkyl)-aryl and —NR¹⁴R^{14′};
each R^{10′} is independently selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

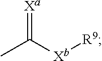

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

In some embodiments, in compounds of Formula (V), R^{2a} is selected from: —CH₃, —CF₃, —F, —Cl, —OCH₃ and —OCF₃.

In some embodiments, in compounds of Formula (V), R^{2a} is selected from: —CF₃, —F, —Cl and —OCH₃.

In some embodiments, in compounds of Formula (V), R^{2a} is F.

In some embodiments, in compounds of Formula (V), R^{20a} is selected from: —H, —C₁-C₆ alkyl, —C₃-C₈ cycloalkyl, —(C₁-C₆ alkyl)-O—R⁵,

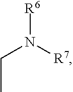

—CO₂R⁸, -aryl, -heteroaryl, —(C₁-C₆ alkyl)-aryl,

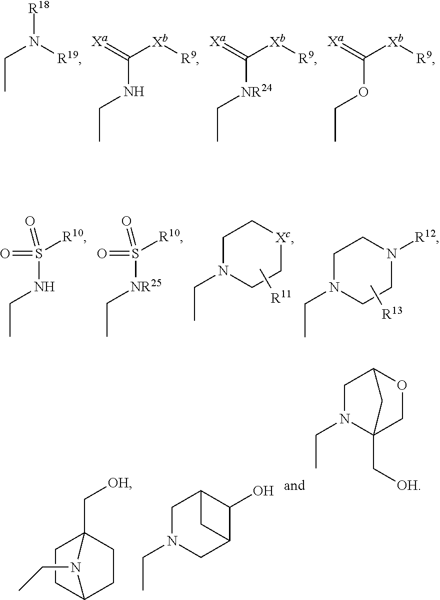

In some embodiments, in compounds of Formula (V), R^{20a} is selected from: —H, —C₁-C₆ alkyl, —(C₁-C₆ alkyl)-O—R⁵,

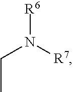

—(C₁-C₆ alkyl)-aryl,

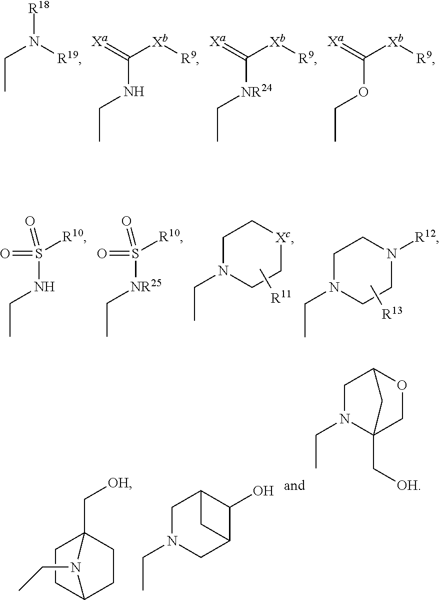

In some embodiments, in compounds of Formula (V), R^{20a} is selected from: —H, —C₁-C₆ alkyl, —(C₁-C₆ alkyl)-O—R⁵,

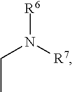

—(C₁-C₆ alkyl)-aryl,

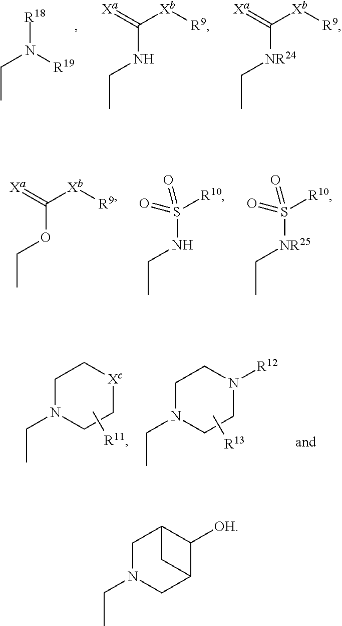

In some embodiments, in compounds of Formula (V), R^{20a} is selected from: —H, —C₁-C₆ alkyl, —(C₁-C₆ alkyl)-O—R⁵,

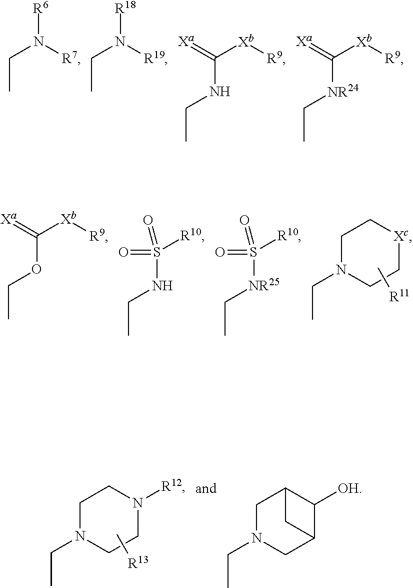

In some embodiments, in compounds of Formula (V), R^{20a} is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

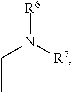

—CO₂R⁸, unsubstituted -aryl, -aminoaryl, -heteroaryl, —(C₁-C₆ alkyl)-aminoaryl

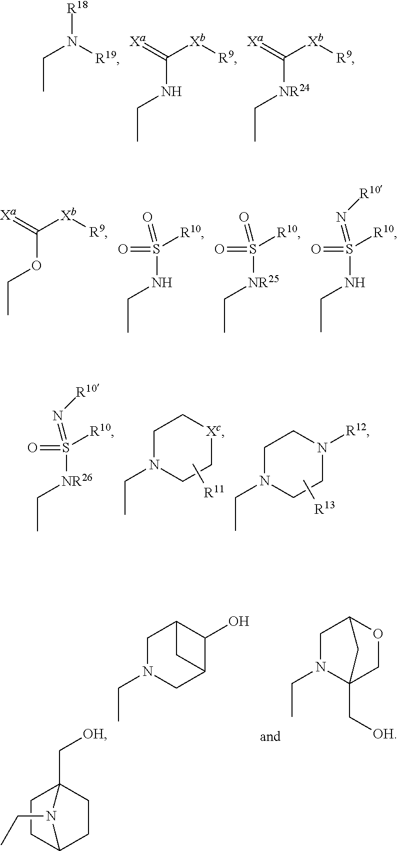

In some embodiments, in compounds of Formula (V), R⁶ and R⁷ are each independently selected from: —H, —C₁-C₆ alkyl, —C₃-C₈ cycloalkyl and —C(O)R¹⁷.

In some embodiments, in compounds of Formula (V), R⁶ is H, and R⁷ is selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (V), R⁶ is H, and R⁷ is selected from: —H, —C₁-C₆ alkyl, —C₃-C₈ cycloalkyl and —C(O)R¹⁷.

In some embodiments, in compounds of Formula (V), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (V), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (V), each R⁹ is independently selected from: —C₁-C₆ alkyl, —C₃-C₈ cycloalkyl, -aryl and —(C₁-C₆ alkyl)-aryl.

In some embodiments, in compounds of Formula (V), each R⁹ is independently selected from: —C₁-C₆ alkyl and —(C₁-C₆ alkyl)-aryl.

In some embodiments, in compounds of Formula (V), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (V), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (V), each R¹⁰ is independently selected from: —C₁-C₆ alkyl, —NR¹⁴R^{14′}, -aryl and —(C₁-C₆ alkyl)-aryl.

In some embodiments, in compounds of Formula (V), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (V), R¹² is selected from: —H, —C₁-C₆ alkyl, -aryl, —(C₁-C₆ alkyl)-aryl and —S(O)₂R¹⁶.

In some embodiments, in compounds of Formula (V), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

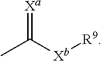

In some embodiments, in compounds of Formula (V), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (V), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (V), R¹⁶ is selected from: -aryl, -heteroaryl and —(C₁-C₆ alkyl)-aryl.

In some embodiments, in compounds of Formula (V), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (V), R¹⁷ is selected from: unsubstituted —C₁-C₆ alkyl, —C₃-C₈ cycloalkyl, —C₃-C₈ heterocycloalkyl, —(C₁-C₆ alkyl)-C₃-C₈ heterocycloalkyl, unsubstituted -aryl, -hydroxyaryl, -aminoaryl, -heteroaryl and —(C₁-C₆ alkyl)-aminoaryl.

In some embodiments, in compounds of Formula (V), R¹⁸ and R¹⁹ taken together with the N atom to which they are bonded form a 4-, 5-, 6-, or 7-membered ring having 0 to 3 substituents selected from: halogen, unsubstituted —C₁-C₆ alkyl, —C₁-C₆ haloalkyl, —C₁-C₆ aminoalkyl, —C₁-C₆ hydroxyalkyl, —C₃-C₈ cycloalkyl and —(C₁-C₆ alkyl)-O—R⁵.

In some embodiments, in compounds of Formula (V), R¹⁷ is —C₁-C₆ alkyl.

In some embodiments, in compounds of Formula (V), X^{a} and X^{b} are each independently selected from: NH and O.

In some embodiments, in compounds of Formula (V), X^{a} and X^{b} are each O.

In some embodiments, in compounds of Formula (V), R^{20a} is —(C₁-C₆ alkyl)-O—R⁵.

In some embodiments, in compounds of Formula (V), R^{20a} is —(C₁-C₆ alkyl)-O—R⁵, and R⁵ is H.

In some embodiments, in compounds of Formula (V), R^{2a} is F; R^{20a} is —(C₁-C₆ alkyl)-O—R⁵, and R⁵ is H.

Other combinations of any of the foregoing embodiments for compounds of Formula (V) are also contemplated and each combination forms a separate embodiment for the purposes of the present disclosure.

Certain embodiments of the present disclosure relate to ADCs having Formula (X), in which D is a compound of Formula (VI):

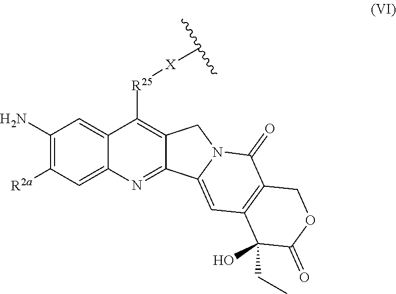

wherein:
R^{2a} is selected from: —H, —CH₃, —CF₃, —F, —Br, —Cl, —OH, —OCH₃ and —OCF₃;
X is —O—, —S— or —NH—, and R²⁵ is selected from: —C₁-C₆ alkyl, —(C₁-C₆ alkyl)-O—R^{5a}, —CO₂R^{8a}, -aryl, -heteroaryl, —(C₁-C₆ alkyl)-aryl,

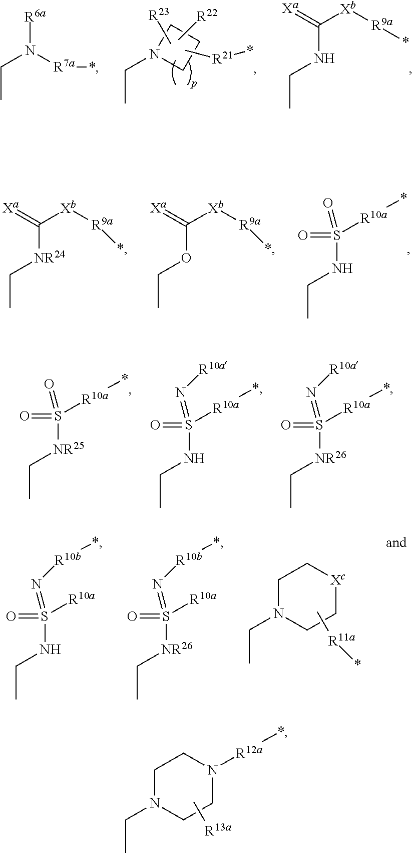

wherein * is the point of attachment to X, and wherein p is 1, 2, 3 or 4; or
X is O, and R²⁵—X— is selected from:

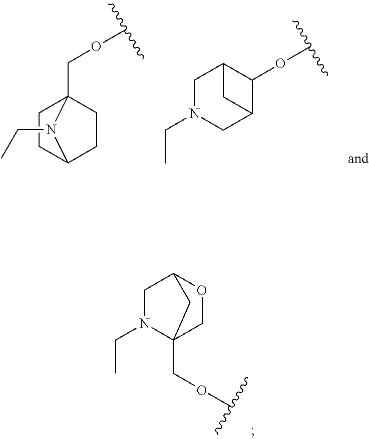 [SMILES: *OCC12CCC(CC1)N2CC ; *OC1C2CC1CN(CC)C2 ; *OCC12COC(CN1CC)C2]

R^{5a} is selected from: —C₁-C₆ alkyl, —C₃-C₈ cycloalkyl, -aryl, -heteroaryl and —(C₁-C₆ alkyl)-aryl;
R^{6a} is selected from: —H, —C₁-C₆ alkyl, —C₃-C₈ cycloalkyl and —C₃-C₈ heterocycloalkyl;
R^{7a} is selected from: —C₁-C₆ alkyl, —C₃-C₈ cycloalkyl, —(C₁-C₆ alkyl)-O—R^{5a}, —C₃-C₈ heterocycloalkyl and —C(O)R^{17a};
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}=X;
each R^{10a} is independently selected from: —C₁-C₆ alkyl, —C₃-C₈ cycloalkyl, -aryl, -heteroaryl, —(C₁-C₆ alkyl)-aryl and

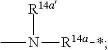

each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, —S(O)₂R^{16a} and

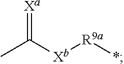

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R^{17a} is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{1a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

In some embodiments, in compounds of Formula (VI), R^{2a} is selected from: —CH₃, —CF₃, —F, —Br, —Cl, —OH, —OCH₃ and —OCF₃.

In some embodiments, in compounds of Formula (VI), R^{2a} is selected from: —CH₃, —CF₃, —F, —Cl, —OCH₃ and —OCF₃.

In some embodiments, in compounds of Formula (VI), R^{2a} is selected from: F and Cl.

In some embodiments, in compounds of Formula (VI), R^{2a} is F.

In some embodiments, in compounds of Formula (VI), X is —O—, —S— or —NH—, and R²⁵ is selected from: —C₁-C₆ alkyl, —(C₁-C₆ alkyl)-O—R^{5a}, —(C₁-C₆ alkyl)-aryl,

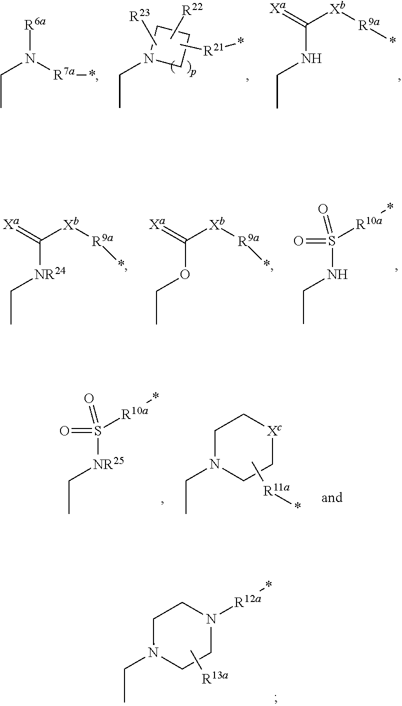

or X is O, and R²⁵—X— is selected from:

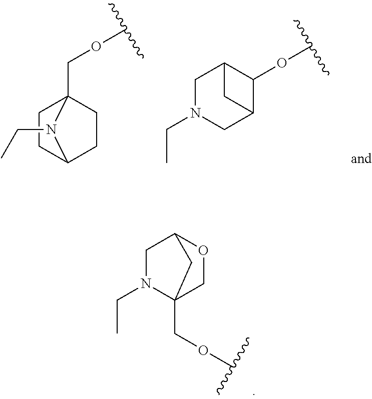 [SMILES: *OCC12CCC(CC1)N2CC ; *OC1C2CC1CN(CC)C2 ; *OCC12COC(CN1CC)C2]

In some embodiments, in compounds of Formula (VI), X is —O—, —S— or —NH—, and R²⁵ is selected from: —C₁-C₆ alkyl, —(C₁-C₆ alkyl)-O—R^{5a}, —(C₁-C₆ alkyl)-aryl,

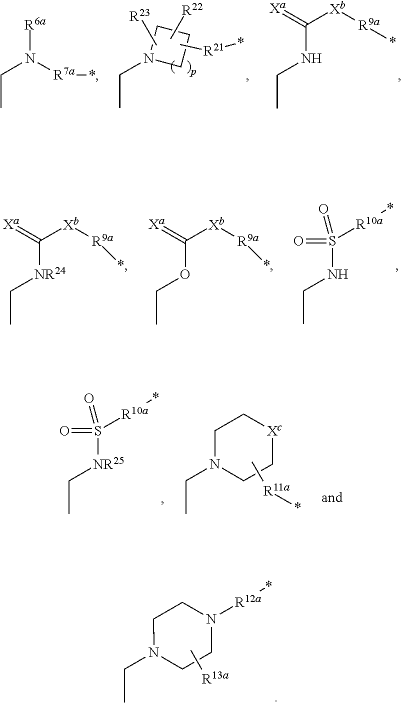

In some embodiments, in compounds of Formula (VI), X is —O—, —S— or —NH—, and R²⁵ is selected from: —C₁-C₆ alkyl, —(C₁-C₆ alkyl)-O—R^{5a},

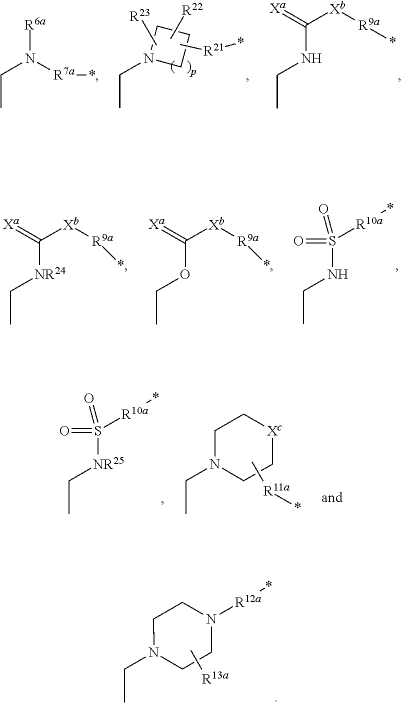

In some embodiments, in compounds of Formula (VI), X is —O—, —S— or —NH—, and R²⁵ is selected from:

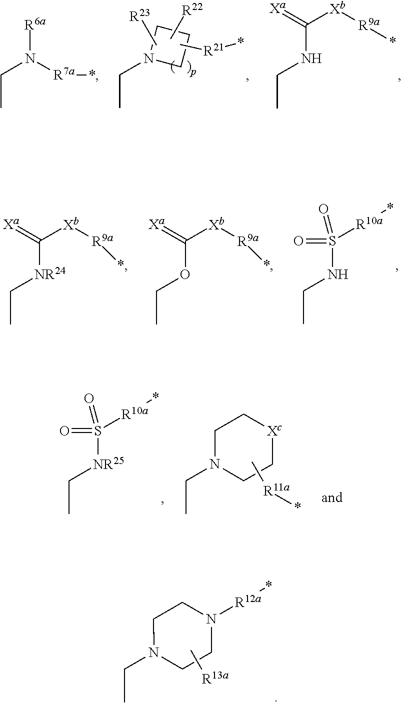

In some embodiments, in compounds of Formula (VI), X is —O— or —NH—.

In some embodiments, in compounds of Formula (VI), R^{6a} is H.

In some embodiments, in compounds of Formula (VI), R^{6a} is selected from: —H, unsubstituted —C₁-C₆ alkyl, —C₁-C₆ haloalkyl, —C₁-C₆ hydroxyalkyl, —C₃-C₈ cycloalkyl and —C₃-C₈ heterocycloalkyl.

In some embodiments, in compounds of Formula (VI), R^{7a} is selected from: —C₁-C₆ alkyl, —C₃-C₈ cycloalkyl and —C(O)R^{17a}

In some embodiments, in compounds of Formula (VI), each R^{9a} is independently selected from: —C₁-C₆ alkyl, —C₃-C₈ cycloalkyl, -aryl and —(C₁-C₆ alkyl)-aryl.

In some embodiments, in compounds of Formula (VI), each R^{9a} is independently selected from: —C₁-C₆ alkyl and —(C₁-C₆ alkyl)-aryl.

In some embodiments, in compounds of Formula (VI), each R^{10a} is independently selected from: —C₁-C₆ alkyl, —C₃-C₈ cycloalkyl, -aryl, —(C₁-C₆ alkyl)-aryl and

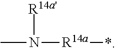

In some embodiments, in compounds of Formula (VI), each R^{10a} is independently selected from: —C₁-C₆ alkyl, -aryl, —(C₁-C₆ alkyl)-aryl and

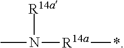

In some embodiments, in compounds of Formula (VI), R^{12a} is selected from: —C₁-C₆ alkyl, -aryl, —(C₁-C₆ alkyl)-aryl and —S(O)₂R^{16a}.

In some embodiments, in compounds of Formula (VI), R^{13a} is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (VI), R^{14a′} is selected from: H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (VI), R^{16a} is selected from: -aryl, -heteroaryl and —(C₁-C₆ alkyl)-aryl.

In some embodiments, in compounds of Formula (VI), R^{17a} is —C₁-C₆ alkyl.

In some embodiments, in compounds of Formula (VI), R²² and R²³ are each independently selected from: —H, -halogen, unsubstituted —C₁-C₆ alkyl, —C₁-C₆ haloalkyl, —C₁-C₆ hydroxyalkyl, —C₁-C₆ aminoalkyl and —C₃-C₈ cycloalkyl.

In some embodiments, in compounds of Formula (VI), X^{a} and X^{b} are each independently selected from: NH and O.

In some embodiments, in compounds of Formula (VI), X^{a} and X^{b} are each O.

In some embodiments, in compounds of Formula (VI), X is O, and R²⁵ is —C₁-C₆ alkyl.

In some embodiments, in compounds of Formula (VI), R^{2a} is F; X is O, and R²⁵ is —C₁-C₆ alkyl.

Other combinations of any of the foregoing embodiments for compounds of Formula (VI) are also contemplated and each combination forms a separate embodiment for the purposes of the present disclosure.

In certain embodiments, each alkyl, cycloalkyl, heterocycloalkyl, aryl and heteroaryl group as defined in any one of Formulae (IV), (V) or (VI) is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl. In some embodiments, each alkyl, cycloalkyl, heterocycloalkyl, aryl and heteroaryl group as defined in any one of Formulae (IV), (V) or (VI) is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

In certain embodiments, in ADCs having Formula (X), D is a compound of Formula (IV), in which R^{1a} is —CH₃, and R^{2a} is F. In some embodiments, in ADCs having Formula (X), D is a compound of Formula (IV), in which R^{1a} is —CH₃; R^{2a} is F; X is —O—; R^{4a} is

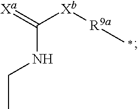

R^{9a} is —C₁-C₆ alkyl, and X^{a} and X^{b} are each O.

In certain embodiments, in ADCs having Formula (X), D is a compound of Formula (V), in which R^{2a} is F, and R^{20a} is H, —(C₁-C₆)—O—R⁵ or

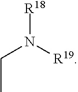

In some embodiments, in ADCs having Formula (X), D is a compound of Formula (V), in which R^{2a} is F; R^{20a} is H, —(C₁-C₆)—O—R⁵ or

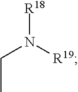

R⁵ is H, and R¹⁸ and R¹⁹ taken together with the N atom to which they are bonded form an unsubstituted 4-, 5-, 6-, or 7-membered ring. In some embodiments, in ADCs having Formula (X), D is a compound of Formula (V), in which R^{2a} is F; R^{20a} is —(C₁-C₆)—O—R⁵, and R⁵ is H.

In certain embodiments, in ADCs having Formula (X), D is a compound of Formula (VI), in which R^{2a} is F; X is —O—, and R²⁵ is —C₁-C₆ alkyl.

### Linker, L

The conjugates of Formula (X) include a linker, L, which is a bifunctional or multifunctional moiety capable of linking one or more camptothecin analogues, D, to the anti-NaPi2b antibody construct, T. A bifunctional (or monovalent) linker, L, links a single compound D to a single site on the anti-NaPi2b antibody construct, T, whereas a multifunctional (or polyvalent) linker, L, links more than one compound, D, to a single site on the anti-NaPi2b antibody construct, T. A linker that links one compound, D, to more than one site on the anti-NaPi2b antibody construct, T, may also be considered to be multifunctional.

Linker, L, includes a functional group capable of reacting with the target group or groups on the anti-NaPi2b antibody construct, T, and at least one functional group capable of reacting with a target group on the camptothecin analogue, D. Suitable functional groups are known in the art and include those described, for example, in Bioconjugate Techniques (G. T. Hermanson, 2013, Academic Press). Groups on the anti-NaPi2b antibody construct, T, and the camptothecin analogue, D, that may serve as target groups for linker attachment include, but are not limited to, thiol, hydroxyl, carboxyl, amine, aldehyde and ketone groups.

Non-limiting examples of functional groups capable of reacting with thiols include maleimide, haloacetamide, haloacetyl, activated esters (such as succinimide esters, 4-nitrophenyl esters, pentafluorophenyl esters and tetrafluorophenyl esters), anhydrides, acid chlorides, sulfonyl chlorides, isocyanates and isothiocyanates. Also useful in this context are “self-stabilizing” maleimides as described in Lyon et al., 2014, Nat. Biotechnol., 32:1059-1062.

Non-limiting examples of functional groups capable of reacting with amines include activated esters (such as N-hydroxysuccinamide (NHS) esters and sulfo-NHS esters), imido esters (such as Traut's reagent), isothiocyanates, aldehydes and acid anhydrides (such as diethylenetriaminepentaacetic anhydride (DTPA)). Other examples include the use of succinimido-1,1,3,3-tetra-methyluronium tetrafluoroborate (TSTU) or benzotriazol-1-yl-oxytripyrrolidinophosphonium hexafluorophosphate (PyBOP) to convert a carboxyl group to an activated ester, which may then be reacted with an amine.

Non-limiting examples of functional groups capable of reacting with an electrophilic group such as an aldehyde or ketone carbonyl group include hydrazide, oxime, amino, hydrazine, thiosemicarbazone, hydrazine carboxylate and arylhydrazide.

In certain embodiments, linker, L, may include a functional group that allows for bridging of two interchain cysteines on the anti-NaPi2b antibody construct, such as a ThioBridge™ linker (Badescu et al., 2014, Bioconjug. Chem. 25:1124-1136), a dithiomaleimide (DTM) linker (Behrens et al., 2015, Mol. Pharm. 12:3986-3998), a dithioaryl(TCEP)pyridazinedione-based linker (Lee et al., 2016, Chem. Sci., 7:799-802) or a dibromopyridazinedione-based linker (Maruani et al., 2015, Nat. Commun., 6:6645).

Alternatively, the anti-NaPi2b antibody construct, T, may be modified to include a non-natural reactive group, such as an azide, that allows for conjugation to the linker via a complementary reactive group on the linker. For example, conjugation of the linker to the anti-NaPi2b antibody construct may make use of click chemistry reactions (see, for example, Chio & Bane, 2020, Methods Mol. Biol., 2078:83-97), such as the azide-alkyne cycloaddition (AAC) reaction, which has been used successfully in the development of antibody-drug conjugates. The AAC reaction may be a copper-catalyzed AAC (CuAAC) reaction, which involves coupling of an azide with a linear alkyne, or a strain-promoted AAC (SPAAC) reaction, which involves coupling of an azide with a cyclooctyne.

Linker, L, may be a cleavable or a non-cleavable linker. A cleavable linker is a linker that is susceptible to cleavage under specific conditions, for example, intracellular conditions (such as in an endosome or lysosome) or within the vicinity of a target cell (such as in the tumor microenvironment). Examples include linkers that are protease-sensitive, acid-sensitive or reduction-sensitive. Non-cleavable linkers by contrast, rely on the degradation of the antibody in the cell, which typically results in the release of an amino acid-linker-drug moiety.

Examples of cleavable linkers include, for example, linkers comprising an amino acid sequence that is a cleavage recognition sequence for a protease. Many such cleavage recognition sequences are known in the art. For conjugates that are not intended to be internalized by a cell, for example, an amino acid sequence that is recognized and cleaved by a protease present in the extracellular matrix in the vicinity of a target cell, such as a cancer cell, may be employed. Examples of extracellular tumor-associated proteases include, for example, plasmin, matrix metalloproteases (MMPs), elastase and kallikrein-related peptidases.

For conjugates intended to be internalized by a cell, linker, L, may comprise an amino acid sequence that is recognized and cleaved by an endosomal or lysosomal protease. Examples of such proteases include, for example, cathepsins B, C, D, H, L and S, and legumain.

Cleavage recognition sequences may be, for example, dipeptides, tripeptides or tetrapeptides. Non-limiting examples of dipeptide recognition sequences that may be included in cleavable linkers include, but are not limited to, Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys. Examples of tri- and tetrapeptide cleavage sequences include, but are not limited to, Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val, Ala-Leu-Ala-Leu, Gly-Phe-Leu-Gly, Gly-Gly-Phe-Gly and Gly-Phe-Gly-Gly.

Additional examples of cleavable linkers include disulfide-containing linkers such as N-succinimydyl-4-(2-pyridyldithio) butanoate (SPDB) and N-succinimydyl-4-(2-pyridyldithio)-2-sulfo butanoate (sulfo-SPDB). Disulfide-containing linkers may optionally include additional groups to provide steric hindrance adjacent to the disulfide bond in order to improve the extracellular stability of the linker, for example, inclusion of a geminal dimethyl group. Other cleavable linkers include linkers hydrolyzable at a specific pH or within a pH range, such as hydrazone linkers. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumor interstitium (see, for example, De Graaf et al., 2002, Curr. Pharm. Des. 8:1391-1403, and International Patent Publication No. WO 2007/011968). β-glucuronide may also function to improve the hydrophilicity of linker, L.

Another example of a linker that is cleaved internally within a cell and improves hydrophilicity is a linker comprising a pyrophosphate diester moiety (see, for example, Kern et al., 2016, J Am Chem Soc., 138:2430-1445).

In certain embodiments, the linker, L, comprised by the conjugate of Formula (X) is a cleavable linker. In some embodiments, linker, L, comprises a cleavage recognition sequence. In some embodiments, linker, L, may comprise an amino acid sequence that is recognized and cleaved by a lysosomal protease.

Cleavable linkers may optionally further comprise one or more additional functionalities such as self-immolative and self-elimination groups, stretchers, or hydrophilic moieties.

Self-immolative and self-elimination groups that find use in linkers include, for example, p-aminobenzyl (PAB) and p-aminobenzyloxycarbonyl (PABC) groups, methylated ethylene diamine (MED) and hemi-aminal groups. Other examples of self-immolative groups include, but are not limited to, aromatic compounds that are electronically similar to the PAB or PABC group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., 1995, Chemistry Biology 2:223-227) and 2-aminophenylpropionic acid amides (Amsberry, et al., 1990, J. Org. Chem. 55:5867-5877). Self-immolative/self-elimination groups are typically attached to an amino or hydroxyl group on the compound, D. Self-immolative/self-elimination groups, alone or in combination are often included in peptide-based linkers, but may also be included in other types of linkers.

Stretchers that find use in linkers for drug conjugates include, for example, alkylene groups and stretchers based on aliphatic acids, diacids, amines or diamines, such as diglycolate, malonate, caproate and caproamide. Other stretchers include, for example, glycine-based stretchers and polyethylene glycol (PEG) or monomethoxy polyethylene glycol (mPEG) stretchers.

PEG and mPEG stretchers can also function as hydrophilic moieties within a linker. For example, PEG or mPEG may be included in a linker either “in-line” or as pendant groups to increase the hydrophilicity of the linker (see, for example, U.S. Patent Application Publication No. US 2016/0310612). Various PEG-containing linkers are commercially available from companies such as Quanta BioDesign, Ltd (Plain City, OH). Other hydrophilic groups that may optionally be incorporated into linker, L, include, for example, 0-glucuronide, sulfonate groups, carboxylate groups and pyrophosphate diesters.

In certain embodiments, ADCs of Formula (X) may comprise a cleavable linker. In some embodiments, ADCs of Formula (X) may comprise a peptide-containing linker. In some embodiments, ADCs of Formula (X) may comprise a protease-cleavable linker.

In some embodiments, in ADCs of Formula (X), m is 1, and linker, L, is a cleavable linker having Formula (XI):

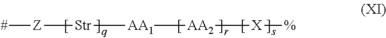

wherein:
Z is a functional group capable of reacting with a target group on the anti-NaPi2b antibody construct, T;
Str is a stretcher;
AA₁ and AA₂ are each independently an amino acid, wherein AA₁-[AA₂]ᵣ forms a protease cleavage site;
X is a self-immolative group;
q is 0 or 1;
r is 1, 2 or 3;
s is 0, 1 or 2;
# is the point of attachment to the anti-NaPi2b antibody construct, T, and % is the point of attachment to the camptothecin analogue, D.

In some embodiments, in linkers of Formula (XI), q is 1.

In some embodiments, in linkers of Formula (XI), s is 1. In some embodiments, in ADCs of Formula (XI), s is 0.

In some embodiments, in linkers of Formula (XI), r is 1. In some embodiments, in ADCs of Formula (XI), r is 3.

In some embodiments, in linkers of Formula (XI):
Z is

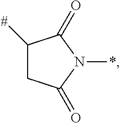

where # is the point of attachment to T, and * is the point of attachment to the remainder of the linker.

In some embodiments, in linkers of Formula (XI), Str is selected from:

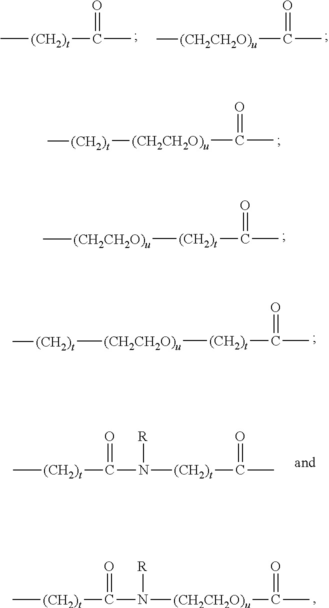

wherein:
R is H or C₁-C₆ alkyl;
t is an integer between 2 and 10, and u is an integer between 1 and 10.

In some embodiments, in linkers of Formula (XI), Str is selected from:

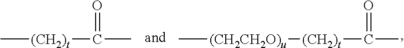

wherein:
t is an integer between 2 and 10, and
u is an integer between 1 and 10.

In some embodiments, in linkers of Formula (XI), AA₁-[AA₂]ᵣ is a dipeptide (i.e. r=1). In some embodiments, in linkers of Formula (XI), AA₁-[AA₂]ᵣ has a sequence selected from: Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys.

In some embodiments, in linkers of Formula (XI), AA₁-[AA₂]ᵣ is a tripeptide (i.e. r=2). In some embodiments, in linkers of Formula (XI), AA₁-[AA₂]ᵣ has a sequence selected from: Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, and Asn-Pro-Val.

In some embodiments, in linkers of Formula (XI), AA₁-[AA₂]ᵣ is a tetrapeptide (i.e. r=3). In some embodiments, in linkers of Formula (XI), AA₁-[AA₂]ᵣ has a sequence selected from: Ala-Leu-Ala-Leu, Gly-Phe-Leu-Gly, Gly-Gly-Phe-Gly and Gly-Phe-Gly-Gly.

In certain embodiments, in ADCs of Formula (X), m is 1, and linker, L, is a cleavable linker having Formula (XII):

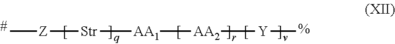

wherein:
Z is a functional group capable of reacting with a target group on the anti-NaPi2b antibody construct, T;
Str is a stretcher;
AA₁ and AA₂ are each independently an amino acid, wherein AA₁-[AA₂]ᵣ forms a protease cleavage site;
Y is —NH—CH₂—;
q is 0 or 1;
r is 1, 2 or 3;
v is 0 or 1;
# is the point of attachment to the anti-NaPi2b antibody construct, T, and
% is the point of attachment to the camptothecin analogue, D.

In some embodiments, in linkers of Formula (XII), q is 1.

In some embodiments, in linkers of Formula (XII), v is 0. In some embodiments, in ADCs of Formula (XII), s is 1.

In some embodiments, in linkers of Formula (XII), r is 1. In some embodiments, in ADCs of Formula (XII), r is 3.

In some embodiments, in linkers of Formula (XII):
Z is

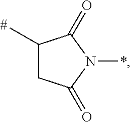

where # is the point of attachment to T, and * is the point of attachment to the remainder of the linker.

In some embodiments, in linkers of Formula (XII), Str is selected from:

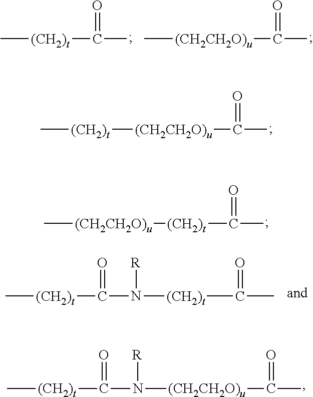

wherein:
R is H or C₁-C₆ alkyl;
t is an integer between 2 and 10, and
u is an integer between 1 and 10.

In some embodiments, in linkers of Formula (XII), Str is selected from:

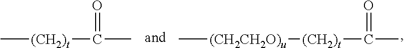

wherein:
t is an integer between 2 and 10, and
u is an integer between 1 and 10.

In some embodiments, in linkers of Formula (XII), AA₁-[AA₂]ᵣ is a dipeptide (i.e. r=1). In some embodiments, in linkers of Formula (XII), AA₁-[AA₂]ᵣ has a sequence selected from: Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys.

In some embodiments, in linkers of Formula (XII), AA₁-[AA₂]ᵣ is a tripeptide (i.e. r=2). In some embodiments, in linkers of Formula (XII), AA₁-[AA₂]ᵣ has a sequence selected from: Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val.

In some embodiments, in linkers of Formula (XII), AA₁-[AA₂]ᵣ is a tetrapeptide (i.e. r=3). In some embodiments, in linkers of Formula (XII), AA₁-[AA₂]ᵣ has a sequence selected from: Ala-Leu-Ala-Leu, Gly-Phe-Leu-Gly, Gly-Gly-Phe-Gly and Gly-Phe-Gly-Gly.

In some embodiments, in linkers of Formula (XII), Y is —NH—CH₂. In some embodiments, in linkers of Formula (XII), v is 1 and Y is —NH—CH₂.

In some embodiments, ADCs of Formula (X) may comprise a disulfide-containing linker. In some embodiments, in ADCs of Formula (X), m is 1, and linker, L, is a cleavable linker having Formula (XIII):

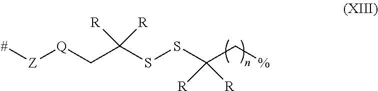

wherein:
Z is a functional group capable of reacting with a target group on the anti-NaPi2b antibody construct, T;
Q is —(CH₂)ₚ— or —(CH₂CH₂O)_{q}—, wherein p and q are each independently an integer between 1 and 10;
each R is independently H or C₁-C₆ alkyl;
n is 1, 2 or 3;
# is the point of attachment to the anti-NaPi2b antibody construct, T, and
% is the point of attachment to the camptothecin analogue, D.

In some embodiments, ADCs of Formula (X) may comprise a β-glucuronide-containing linker.

Various non-cleavable linkers are known in the art for linking drugs to targeting moieties and may be useful in the ADCs of the present disclosure in certain embodiments. Examples of non-cleavable linkers include linkers having an N-succinimidyl ester or N-sulfosuccinimidyl ester moiety for reaction with the anti-NaPi2b antibody construct, as well as a maleimido- or haloacetyl-based moiety for reaction with the camptothecin analogue, or vice versa. An example of such a non-cleavable linker is based on sulfosuccinimidyl-4-[N-maleimidomethyl]cyclohexane-1-carboxylate (sulfo-SMCC). Sulfo-SMCC conjugation typically occurs via a maleimide group which reacts with sulfhydryls (thiols, SH) on the camptothecin analogue, while the sulfo-NHS ester is reactive toward primary amines (as found in lysine and at the N-terminus of proteins or peptides) on the anti-NaPi2b antibody construct. Other non-limiting examples of such linkers include those based on N-succinimidyl 4-(maleimidomethyl)cyclohexanecarboxylate (SMCC), N-succinimidyl-4-(N-maleimidomethyl)-cyclohexane-1-carboxy-(6-amidocaproate) (“long chain” SMCC or LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB) and N-(p-maleimidophenyl)isocyanate (PMPI). Other examples include those comprising a haloacetyl-based functional group such as N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido)propionate (SBAP).

Non-limiting examples of drug-linkers comprising camptothecin analogues of Formula (I) are shown in Table 8, Table 9, and Table 10. Non-limiting examples of conjugates comprising these drug-linkers are shown in Table 11, Table 12 and Table 13. In certain embodiments, the ADC of Formula (X) comprises a drug-linker selected from the drug-linkers shown in Tables 8, 9 and 10. In certain embodiments, the ADC of Formula (X) is selected from the conjugates shown in Tables 11, 12 and 13, where T is the anti-NaPi2b antibody construct and n is between 1 and 10. In some embodiments, the ADC of Formula (X) is selected from the conjugates shown in Tables 11, 12 and 13, where T is the anti-NaPi2b antibody construct and n is between 2 and 8. In some embodiments, the ADC of Formula (X) is selected from the conjugates shown in Tables 11, 12 and 13, where T is the anti-FRα antibody construct and n is between 4 and 8.

In certain embodiments, the ADC of Formula (X) comprises a drug-linker (L-(D)ₘ) selected from MT-GGFG-AM-Compound 139, MC-GGFG-AM-Compound 139, MT-GGFG-Compound 140, MC-GGFG-Compound 140, MT-GGFG-AM-Compound 141, MC-GGFG-AM-Compound 141, MT-GGFG-Compound 141, MC-GGFG-Compound 141, MT-GGFG-Compound 148 and MC-GGFG-Compound 148, and n is 4 or 8. In some embodiments, the ADC of Formula (X) comprises a drug-linker (L-(D)ₘ) selected from MT-GGFG-AM-Compound 139, MC-GGFG-AM-Compound 139, MT-GGFG-Compound 140, MC-GGFG-Compound 140, MT-GGFG-AM-Compound 141, MC-GGFG-AM-Compound 141, MT-GGFG-Compound 141, MC-GGFG-Compound 141, MT-GGFG-Compound 148 and MC-GGFG-Compound 148, and n is 8.

### Preparation of ADCs

ADCs of Formula (X) may be prepared by standard methods known in the art (see, for example, Bioconjugate Techniques (G. T. Hermanson, 2013, Academic Press)). Various linkers and linker components are commercially available or may be prepared using standard synthetic organic chemistry techniques (see, for example, March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press)). In addition, various antibody drug conjugation services are available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramal Pharma Solutions (Grangemouth, UK).

Typically, preparation of the ADCs comprises first preparing a drug-linker, D-L, comprising one or more camptothecin analogues of Formula (I) and linker L, and then conjugating the drug-linker, D-L, to an appropriate group on the anti-NaPi2b antibody construct, T. Ligation of linker, L, to the anti-NaPi2b antibody construct, T, and subsequent ligation of the anti-NaPi2b antibody construct-linker, T-L, to one or more camptothecin analogues of Formula (I), D, remains however an alternative approach that may be employed in some embodiments.

Suitable groups on compounds of Formula (I), D, for attachment of linker, L, in either of the above approaches include, but are not limited to, thiol groups, amine groups, carboxylic acid groups and hydroxyl groups. In some embodiments of the present disclosure, linker, L, is attached to a compound of Formula (I), D, via a hydroxyl or amine group on the compound.

Suitable groups on the anti-NaPi2b antibody construct, T, for attachment of linker, L, in either of the above approaches include sulfhydryl groups (for example, on the side-chain of cysteine residues), amino groups (for example, on the side-chain of lysine residues), carboxylic acid groups (for example, on the side-chains of aspartate or glutamate residues), and carbohydrate groups.

For example, the anti-NaPi2b antibody construct T may comprise one or more naturally occurring sulfhydryl groups allowing the anti-NaPi2b antibody construct, T, to bond to linker, L, via the sulfur atom of a sulfhydryl group. Alternatively, the anti-NaPi2b antibody construct, T, may comprise one or more lysine residues that can be chemically modified to introduce one or more sulfhydryl groups. Reagents that can be used to modify lysine residues include, but are not limited to, N-succinimidyl S-acetylthioacetate (SATA), N-succinimidyl-3-(2-pyridyldithio)propionate (“SPDP”) and 2-iminothiolane hydrochloride (Traut's Reagent). Alternatively, the anti-NaPi2b antibody construct, T, may comprise one or more carbohydrate groups that can be chemically modified to include one or more sulfhydryl groups.

Carbohydrate groups on the anti-NaPi2b antibody construct, T, may also be oxidized to provide an aldehyde (—CHO) group (see, for example, Laguzza et al., 1989, J. Med. Chem. 32(3):548-55), which could subsequently be reacted with linker, L, for example, via a hydrazine or hydroxylamine group on linker, L.

The anti-NaPi2b antibody construct, T, may also be modified to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethyl-L-phenylalanine (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361), to allow for site-specific conjugation. Alternatively, the anti-NaPi2b antibody construct, T, may be modified to include a non-natural reactive group, such as an azide, that allows for conjugation to the linker via a complementary reactive group on the linker, for example, for example, by click chemistry (see, for example, Chio & Bane, 2020, Methods Mol. Biol., 2078:83-97). A further option is the use of GlycoConnect™ technology (Synaffix BV, Nijmegen, Netherlands), which involves enzymatic remodelling of the antibody glycans to allow for attachment of a linker by metal-free click chemistry (see, for example, European Patent No. EP 2 911 699).

Other protocols for the modification of proteins for the attachment or association of linker, L, are known in the art and include those described in Coligan et al., Current Protocols in Protein Science, vol. 2, John Wiley & Sons (2002).

Alternatively, ADCs may be prepared using the enzyme transglutaminase, in particular, bacterial transglutaminase (BTG) from Streptomyces mobaraensis (see, for example, Jeger et al., 2010, Angew. Chem. Int. Ed., 49:9995-9997). BTG forms an amide bond between the side chain carboxamide of a glutamine (the amine acceptor, typically on the antibody) and an alkyleneamino group (the amine donor, typically on the drug-linker), which can be, for example, the F-amino group of a lysine or a 5-amino-n-pentyl group. Antibodies may also be modified to include a glutamine containing peptide, or “tag,” which allows BTG conjugation to be used to conjugate the antibody to a drug-linker (see, for example, U.S. Patent Application Publication No. US 2013/0230543 and International (PCT) Publication No. WO 2016/144608).

A similar conjugation approach utilizes the enzyme sortase A. In this approach, the antibody is typically modified to include the sortase A recognition motif (LPXTG, where X is any natural amino acid) and the drug-linker is designed to include an oligoglycine motif (typically GGG) to allow for sortase A-mediated transpeptidation (see, for example, Beerli, et al., 2015, PLos One, 10:e0131177; Chen et al., 2016, Nature: Scientific Reports, 6:31899).

Once conjugation is complete, the average number of compounds of Formula (I) conjugated to the anti-NaPi2b antibody construct, T, (i.e. the “drug-to-antibody ratio” or DAR) may be determined by standard techniques such as UV/VIS spectroscopic analysis, ELISA-based techniques, chromatography techniques such as hydrophobic interaction chromatography (HIC), UV-MALDI mass spectrometry (MS) and MALDI-TOF MS. In addition, distribution of drug-linked forms (for example, the fraction of the anti-NaPi2b antibody construct, T, containing zero, one, two, three, etc. compounds of Formula (I), D) may also optionally be analyzed. Various techniques are known in the art to measure DAR distribution, including MS (with or without an accompanying chromatographic separation step), hydrophobic interaction chromatography, reverse-phase HPLC or iso-electric focusing gel electrophoresis (IEF) (see, for example, Wakankar et al., 2011, mAbs, 3:161-172).

### Anti-NaPi2b Fc-Silenced ADCs

Target-independent uptake of ADCs and subsequent processing and release of the cytotoxic payload in normal tissues can contribute to the toxicity profile of an ADC. One mechanism of target independent uptake is through FcγR binding of the Fc of the ADCs. Silencing of the Fc binding to FcγRs through specific amino acid mutations can reduce binding to FcγRs, reduce uptake into FcγR-expressing cells, and potentially alter the toxicity profile. As demonstrated and described herein, target-independent binding, i.e., binding to cells that do not express NaPi2b, e.g., binding of the Fc region to one or more Fc receptors, of the Fc-silenced anti-NaPi2b ADCs of the present disclosure is lower compared to the binding of Fc WT anti-NaPi2b ADCs. In some embodiments, the Fc-silenced anti-NaPi2b antibody construct and corresponding ADCs comprising LALADS amino acid substitutions in the Fc region exhibits reduced ADCC and/or ADCP when compared to a reference anti-NaPi2b antibody construct that comprises a wildtype Fc region (e.g., an Fc region that does not comprise LALADS amino acid substitutions). Without being bound by any theory, the reduced ADCC and/or ADCP as well as reduced binding to NaPi2b-negative cells of the Fc-silenced anti-NaPi2b antibody construct comprising LALADS amino acid substitutions in the Fc region demonstrates reduced off-target cell engagement and may indicate the potential for reduced toxicity in vivo.

In one embodiment, the anti-NaPi2b Fc-silenced ADC comprises the structure:

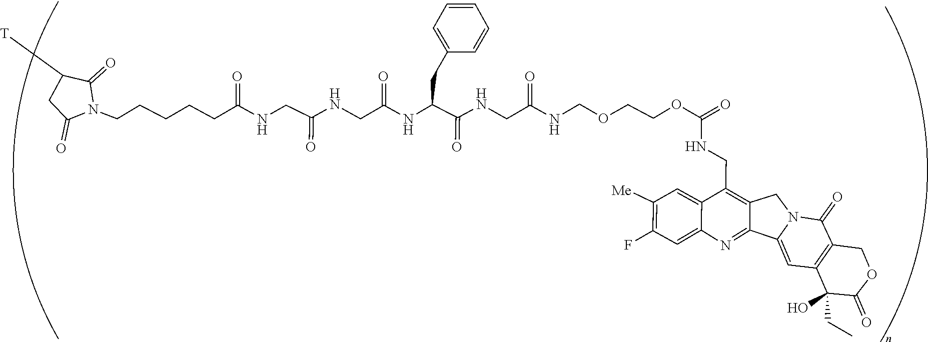

wherein n is between 2 and 6; and
T is an anti-NaPi2b (sodium-dependent phosphate transport protein 2B) antibody construct comprising an antigen-binding domain that binds to human NaPi2b, wherein the anti-NaPi2b antibody construct comprises two heavy chains comprising the sequence as set forth in SEQ ID NO:68, and two light chains comprising the sequence as set forth in SEQ ID NO:67, or wherein the anti-NaPi2b antibody construct comprises two heavy chains comprising the sequence as set forth in SEQ ID NO:66, and two light chains comprising the sequence as set forth in SEQ ID NO:67. In related embodiments, the anti-NaPi2b Fc-silenced ADC comprises the above structure, but wherein n is between 3.5 and 4.5. In other related embodiments, the anti-NaPi2b Fc-silenced ADC comprises the above structure, but wherein n is about 4. In various embodiments, such anti-NaPi2b Fc-silenced ADC can also be described herein as “v40502 (LALADS)-MC-GGFG-AM-Compound 139 ADC.”

The anti-NaPi2b Fc-silenced ADCs described herein, comprising LALADS mutations in the Fc, bind to cancer cells expressing NaPi2b, and/or are internalized by cancer cells expressing NaPi2b, and/or are cytotoxic to cancer cells expressing NaPi2b, and/or have anti-tumor activity in xenograft models of NaPi2b-expressing cancers. In certain embodiments, these anti-NaPi2b Fc-silenced ADCs also exhibit reduced target-independent binding to normal cells that express one or more FcγRs when compared to comparable ADCs that comprise a WT Fc region. In certain embodiments, an anti-NaPi2b Fc-silenced ADC of the present disclosure also exhibits one or more in vivo properties that are different compared to that of a comparable ADC that contains a WT Fc region, e.g., one or more of a different PK profile (e.g., longer clearance times) and different anti-tumor activities in various tumor models.

### Pharmaceutical Compositions

For therapeutic uses, the ADCs of the present disclosure are typically formulated as pharmaceutical compositions. Certain embodiments of the present disclosure thus relate to pharmaceutical compositions comprising an ADC as described herein and a pharmaceutically acceptable carrier, diluent, or excipient. Such pharmaceutical compositions may be prepared by known procedures using well-known and readily available ingredients.

Pharmaceutical compositions may be formulated for administration to a subject by, for example, oral (including, for example, buccal or sublingual), topical, parenteral, rectal or vaginal routes, or by inhalation or spray. The term “parenteral” as used herein includes subcutaneous injection, and intradermal, intra-articular, intravenous, intramuscular, intravascular, intrasternal, intrathecal injection or infusion. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

In certain embodiments, the pharmaceutical compositions comprising the ADCs are formulated for parenteral administration, for example as lyophilized formulations or aqueous solutions. Such pharmaceutical compositions may be provided, for example, in a unit dosage injectable form.

Pharmaceutically acceptable carriers are generally nontoxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions comprising the ADCs may be in the form of a sterile injectable aqueous or oleaginous solution or suspension. Such suspensions may be formulated using suitable dispersing or wetting agents and/or suspending agent that are known in the art. The sterile injectable solution or suspension may comprise the ADC in a non-toxic parentally acceptable diluent or carrier. Acceptable diluents and carriers that may be employed include, for example, 1,3-butanediol, water, Ringer's solution or isotonic sodium chloride solution. In addition, sterile, fixed oils may be employed as a carrier. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents may also be included in the injectable solution or suspension.

In certain embodiments, the composition comprising the ADC may be formulated for intravenous administration to humans. Typically, compositions for intravenous administration are solutions in sterile isotonic aqueous buffer. Where necessary, the composition may also include a solubilizing agent and/or a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### METHODS OF USE

Certain embodiments of the present disclosure relate to the therapeutic use of the ADCs described herein. Some embodiments relate to the use of the ADCs as therapeutic agents.

Certain embodiments of the present disclosure relate to methods of inhibiting abnormal cancer cell or tumor cell growth; inhibiting cancer cell or tumor cell proliferation, or treating cancer in a subject, comprising administering an ADC described herein. In certain embodiments, the ADCs described herein may be used in the treatment of cancer. Some embodiments of the present disclosure thus relate to the use of the ADCs as anti-cancer agents.

Certain embodiments of the present disclosure relate to methods of inhibiting the proliferation of cancer or tumor cells comprising contacting the cells with an ADC as described herein, for example, an ADC of Formula (X). Some embodiments relate to a method of killing cancer or tumor cells comprising contacting the cells with an ADC as described herein, for example, an ADC of Formula (X).

Some embodiments relate to methods of treating a subject having a cancer by administering to the subject an ADC as described herein, for example, an ADC of Formula (X). In this context, treating the subject may result in one or more of a reduction in the size of a tumor, the slowing or prevention of an increase in the size of a tumor, an increase in the disease-free survival time between the disappearance or removal of a tumor and its reappearance, prevention of a subsequent occurrence of a tumor (for example, metastasis), an increase in the time to progression, reduction of one or more adverse symptom associated with a tumor, and/or an increase in the overall survival time of a subject having cancer.

Certain embodiments relate to the use of an ADC as described herein, for example, an ADC of Formula (X), in a method of inhibiting tumor growth in a subject. Some embodiments relate to the use of an ADC as described herein, for example, an ADC of Formula (X), in a method of inhibiting proliferation of and/or killing cancer cells in vitro. Some embodiments relate to the use of an ADC as described herein, for example, an ADC of Formula (X), in a method of inhibiting proliferation of and/or killing cancer cells in vivo in a subject having a cancer.

Examples of cancers which may be treated in certain embodiments are carcinomas, including adenocarcinomas and squamous cell carcinomas; melanomas and sarcomas. Carcinomas and sarcomas are also frequently referred to as “solid tumors.” Examples of commonly occurring solid tumors that may be treated in certain embodiments include, but are not limited to, brain cancer, breast cancer, cervical cancer, colon cancer, head and neck cancer, kidney cancer, lung cancer, ovarian cancer, pancreatic cancer, prostate cancer, stomach cancer, uterine cancer, non-small cell lung cancer (NSCLC) and colorectal cancer. Various forms of lymphoma also may result in the formation of a solid tumor and, therefore, may also be considered to be solid tumors in certain situations. Typically, the cancer to be treated is an NaPi2b-expressing cancer.

Certain embodiments relate to methods of inhibiting the growth of NaPi2b-positive tumor cells comprising contacting the cells with an ADC as described herein, for example, an ADC of Formula (X). The cells may be in vitro or in vivo. In certain embodiments, the ADCs may be used in methods of treating an NaPi2b-positive cancer or tumor in a subject.

Cancers that overexpress NaPi2b are typically solid tumors. Examples include, but are not limited to, ovarian cancer, endometrial cancer, and lung cancers (such as non-small cell lung cancer (NSCLC). In one embodiment, an ADC as described herein may be used in a method of treating ovarian cancer or lung cancer. In one embodiment, an ADC as described herein may be used in a method of treating NSCLC.

### Pharmaceutical Kits

Certain embodiments relate to pharmaceutical kits comprising an ADC as described herein, for example, an ADC of Formula (X).

The kit typically will comprise a container holding the ADC and a label and/or package insert on or associated with the container. The label or package insert contains instructions customarily included in commercial packages of therapeutic products, providing information about the indications, usage, dosage, administration, contraindications and/or warnings concerning the use of such therapeutic products. The label or package insert may further include a notice in the form prescribed by a governmental agency regulating the manufacture, use or sale of pharmaceuticals or biological products, which notice reflects approval by the agency of manufacture, for use or sale for human or animal administration. In some embodiments, the container may have a sterile access port. For example, the container may be an intravenous solution bag or a vial having a stopper that may be pierced by a hypodermic injection needle.

In addition to the container holding the ADC, the kit may optionally comprise one or more additional containers comprising other components of the kit. For example, a pharmaceutically acceptable buffer (such as bacteriostatic water for injection (BWFI), phosphate-buffered saline, Ringer's solution or dextrose solution), other buffers or diluents.

Suitable containers include, for example, bottles, vials, syringes, intravenous solution bags, and the like. The containers may be formed from a variety of materials such as glass or plastic. If appropriate, one or more components of the kit may be lyophilized or provided in a dry form, such as a powder or granules, and the kit can additionally contain a suitable solvent for reconstitution of the lyophilized or dried component(s).

The kit may further include other materials desirable from a commercial or user standpoint, such as filters, needles, and syringes.

### Tables 8 to 13

TABLE 8
Exemplary drug-linker (DL) structures comprising camptothecin analogues of Formula (I) with a C7 linkage

| Name | Structure |
|---|---|
| DL: MT- GGFG- Compound 104 | 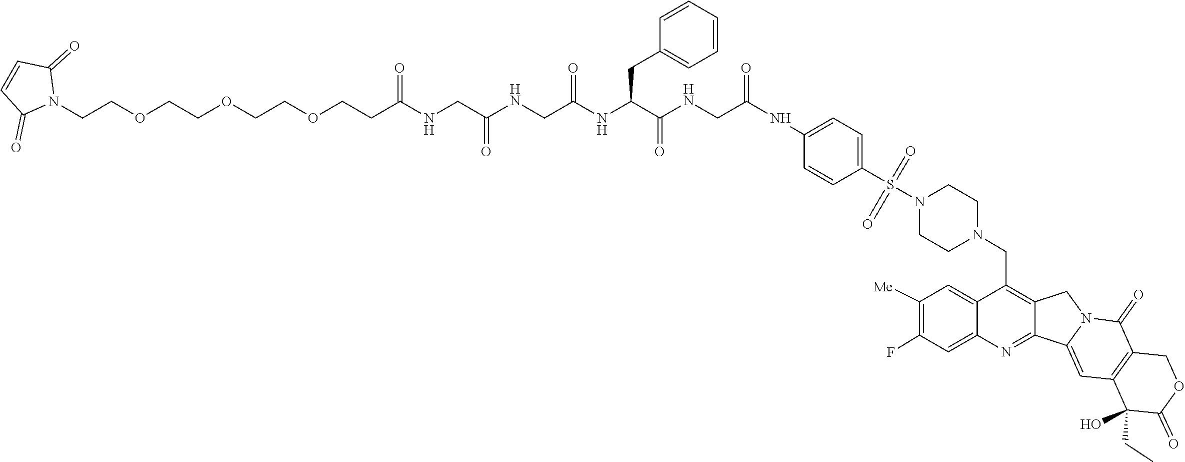 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)CCOCCOCCOCCN3C(=O)C=CC3=O)cc2)CC1] |
| DL: MT- GGFG- Compound 108 | 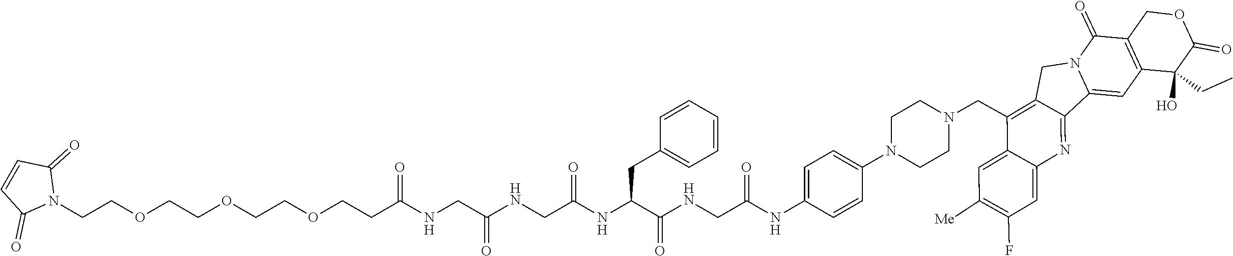 [SMILES: CC[C@@]1(O)C(=O)OCc2c1cc1n(c2=O)Cc2c-1nc1cc(F)c(C)cc1c2CN1CCN(c2ccc(NC(=O)CNC(=O)[C@H](Cc3ccccc3)NC(=O)CNC(=O)CNC(=O)CCOCCOCCOCCN3C(=O)C=CC3=O)cc2)CC1] |
| DL: MT- GGFG- Compound 127 | 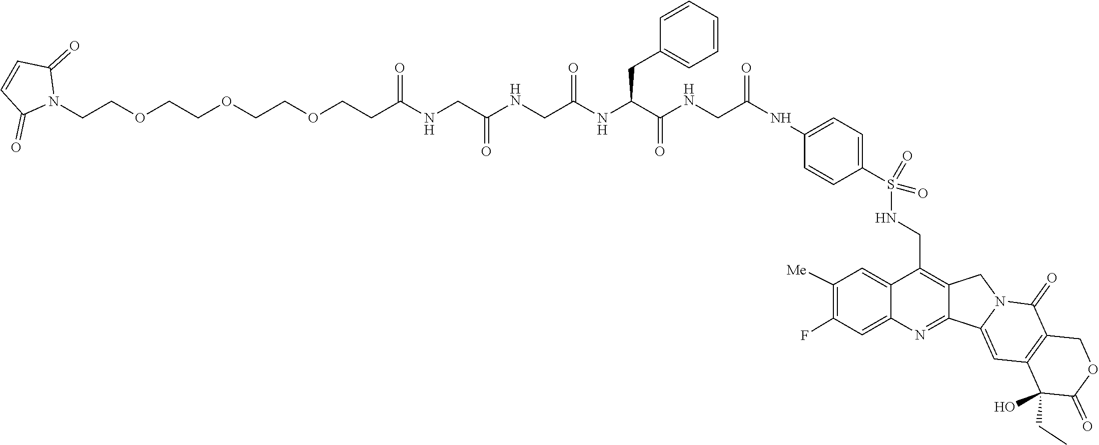 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)CCOCCOCCOCCN2C(=O)C=CC2=O)cc1] |
| DL: MT- GGFG- AM- Compound 136 | 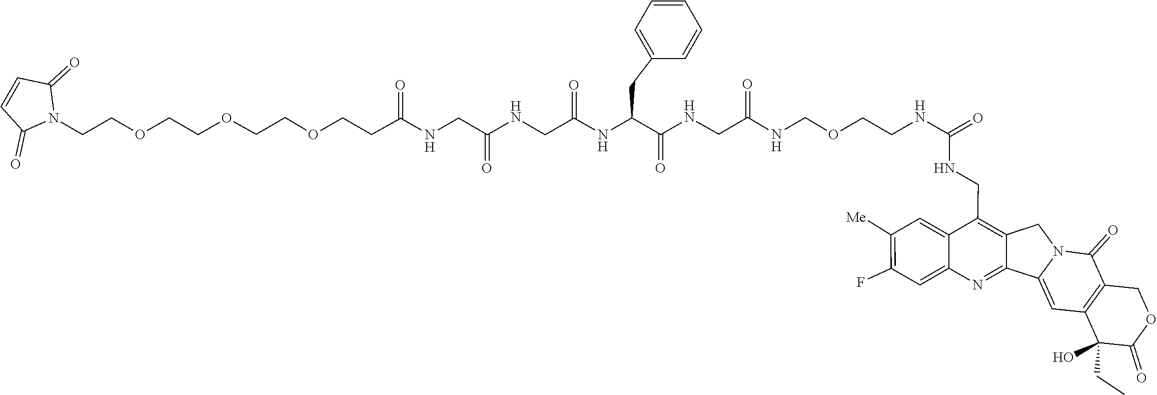 [SMILES: CC[C@@]1(O)C(=O)OCc2c1cc1n(c2=O)Cc2c-1nc1cc(F)c(C)cc1c2CNC(=O)NCCOCNC(=O)CNC(=O)[C@H](Cc1ccccc1)NC(=O)CNC(=O)CNC(=O)CCOCCOCCOCCN1C(=O)C=CC1=O] |
| DL: MT- GGFG- AM- Compound 139 | 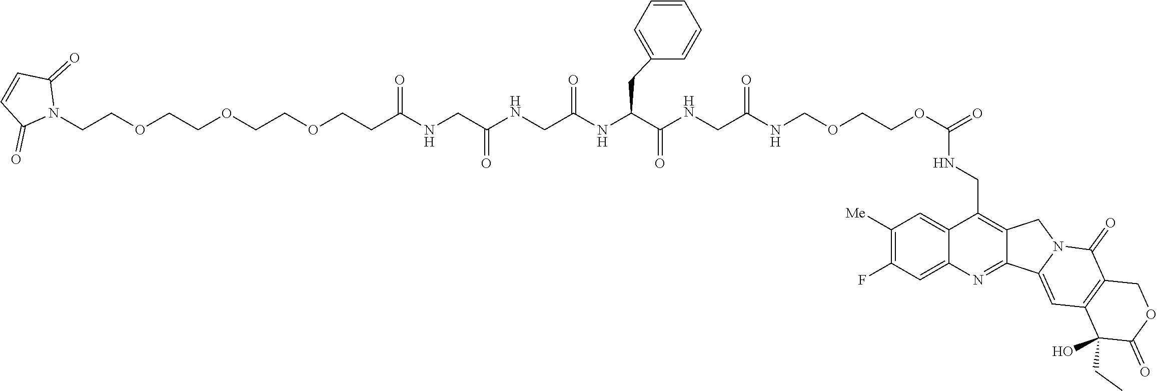 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCOCCOCCOCCN1C(=O)C=CC1=O] |
| DL: MT- GGFG- AM- Compound 129 | 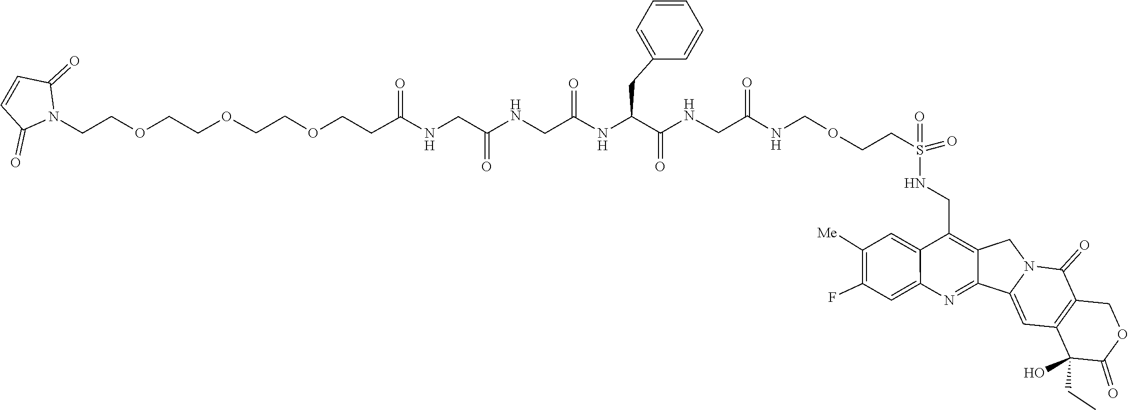 [SMILES: CC[C@@]1(O)C(=O)OCc2c1cc1n(c2=O)Cc2c-1nc1cc(F)c(C)cc1c2CNS(=O)(=O)CCOCNC(=O)CNC(=O)[C@H](Cc1ccccc1)NC(=O)CNC(=O)CNC(=O)CCOCCOCCOCCN1C(=O)C=CC1=O] |
| DL: MT- GGFG- AM- Compound 113 | 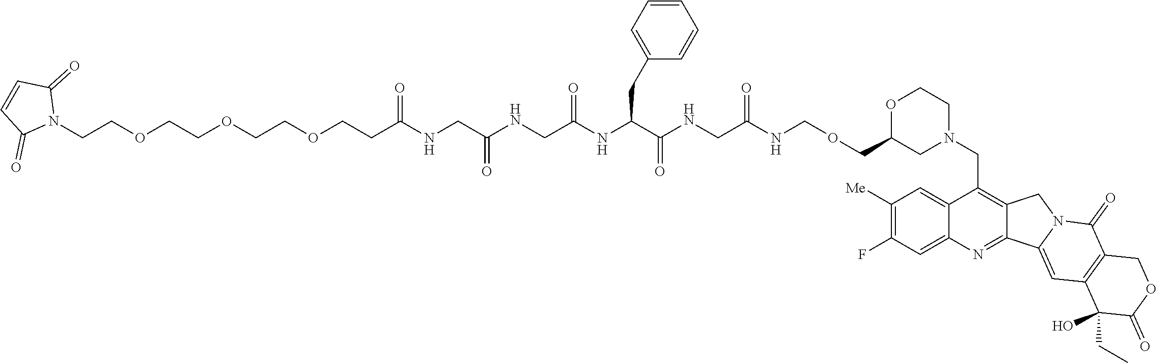 [SMILES: CC[C@@]1(O)C(=O)OCc2c1cc1n(c2=O)Cc2c-1nc1cc(F)c(C)cc1c2CN1CCO[C@H](COCNC(=O)CNC(=O)[C@H](Cc2ccccc2)NC(=O)CNC(=O)CNC(=O)CCOCCOCCOCCN2C(=O)C=CC2=O)C1] |
| DL: MT- GGFG- AM- Compound 141 | 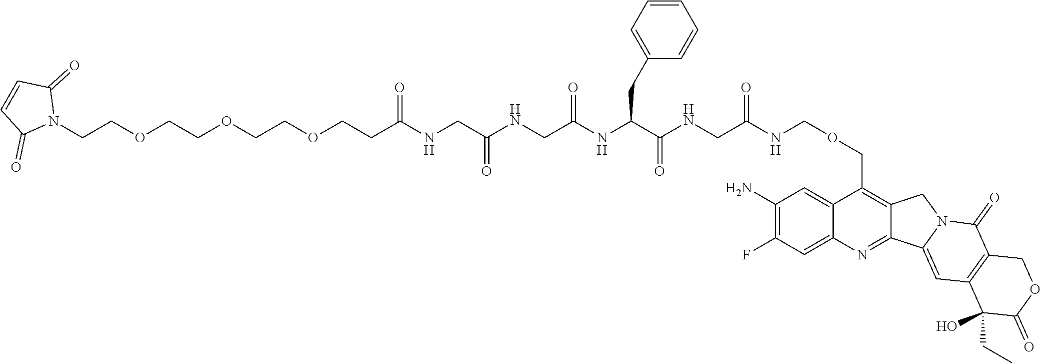 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCOCCOCCOCCN1C(=O)C=CC1=O] |
| DL: MC- GGFG- AM- Compound 141 | 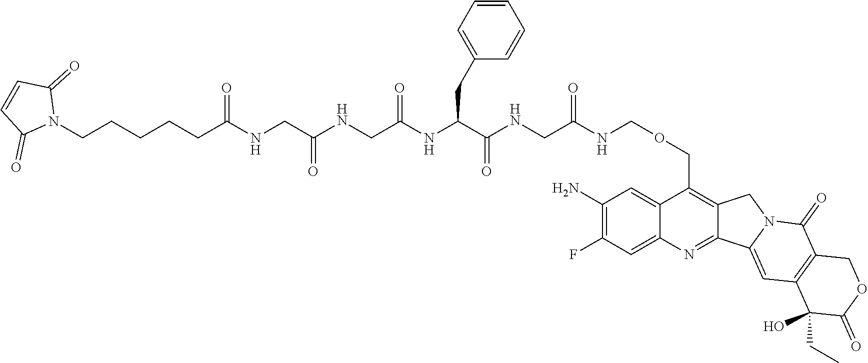 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCCCCN1C(=O)C=CC1=O] |
| DL: MT- GGFG- AM- Compound 117 | 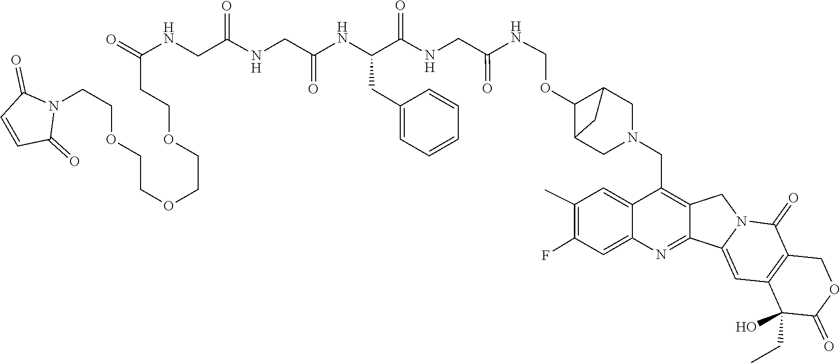 [SMILES: CC[C@@]1(O)C(=O)OCc2c1cc1n(c2=O)Cc2c-1nc1cc(F)c(C)cc1c2CN1CC2CC(C1)C2OCNC(=O)CNC(=O)[C@H](Cc1ccccc1)NC(=O)CNC(=O)CNC(=O)CCOCCOCCOCCN1C(=O)C=CC1=O] |
| DL: MT- GGFG- AM- Compound 118 | 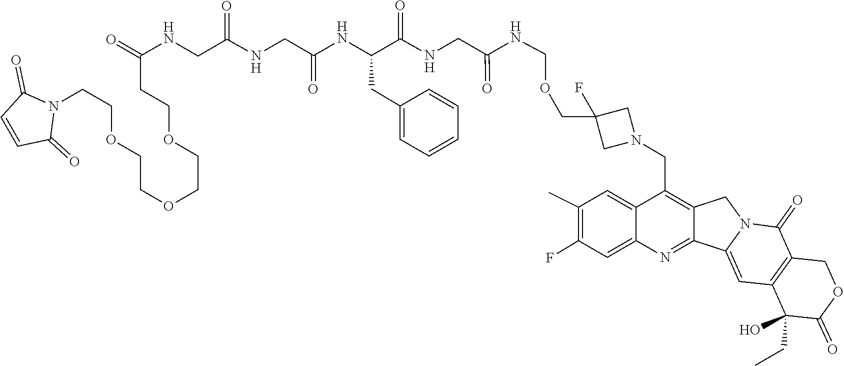 [SMILES: CC[C@@]1(O)C(=O)OCc2c1cc1n(c2=O)Cc2c-1nc1cc(F)c(C)cc1c2CN1CC(F)(COCNC(=O)CNC(=O)[C@H](Cc2ccccc2)NC(=O)CNC(=O)CNC(=O)CCOCCOCCOCCN2C(=O)C=CC2=O)C1] |
| DL: MC- GGFG- AM- Compound 139 | 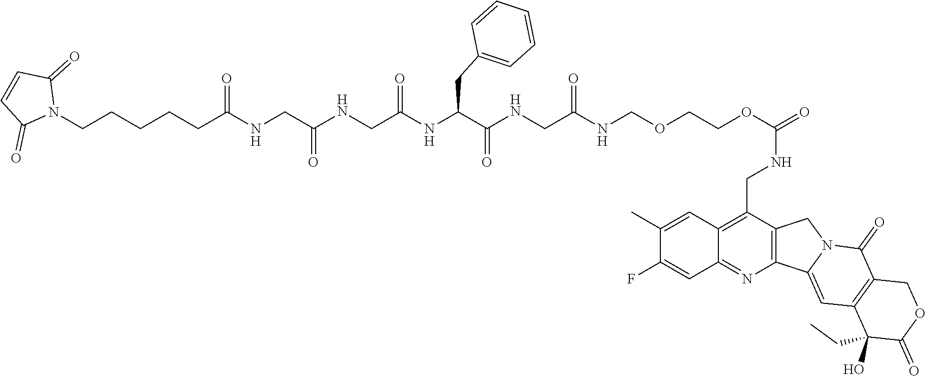 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCCCCN1C(=O)C=CC1=O] |
| DL: DiS- Compound 145 | 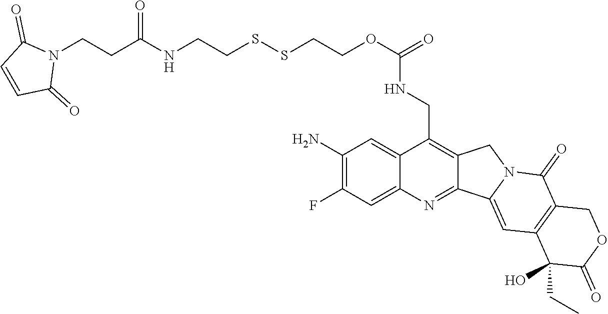 [SMILES: CC[C@@]1(O)C(=O)OCc2c1cc1n(c2=O)Cc2c-1nc1cc(F)c(N)cc1c2CNC(=O)OCCSSCCNC(=O)CCN1C(=O)C=CC1=O] |

TABLE 9
Exemplary drug-linker (DL) structures comprising camptothecin analogues of Formula (I) with a C10 linkage

| Name | Structure |
|---|---|
| DL: MT- GGFG- Compound 140 | 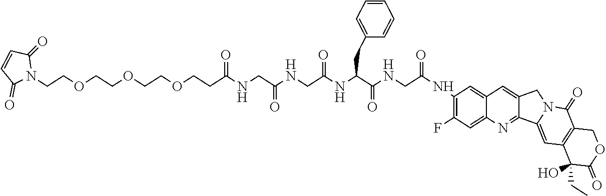 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCOCCOCCOCCN4C(=O)C=CC4=O)c(F)cc3nc2-1] |
| DL: MT- GGFG- Compound 141 | 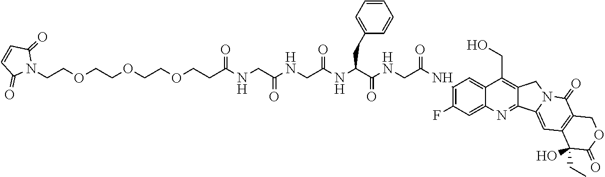 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CO] |
| DL: MT- GGFG- Compound 145 | 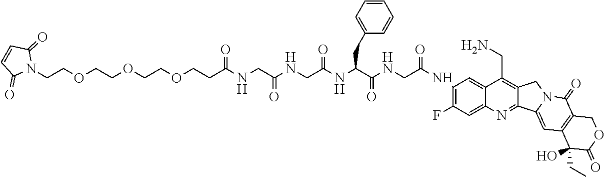 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN] |
| DL: MT- GGFG- Compound 148 | 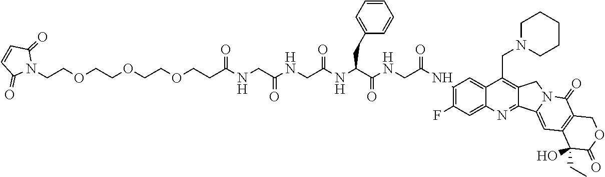 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCCCC1] |
| DL: MC- GGFG- Compound 140 | 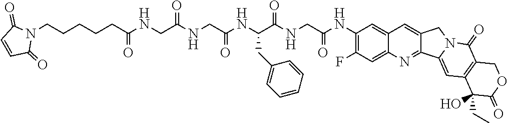 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCCCN4C(=O)C=CC4=O)c(F)cc3nc2-1] |
| DL: MT- GGFG- Compound 142 | 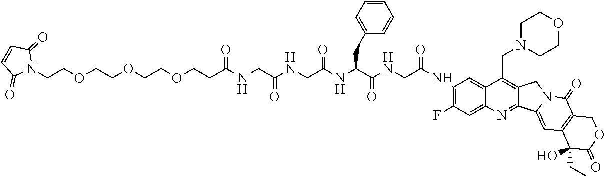 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCOCC1] |
| DL: MC- GGFG- Compound 141 | 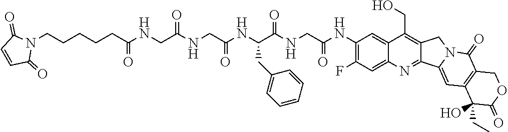 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCCCCN3C(=O)C=CC3=O)cc1c2CO] |
| DL: MC- VA- Compound 140 (DL: 201) | 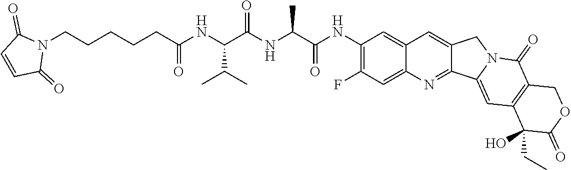 [SMILES: CC[C@@]1(O)C(=O)OCc2c1cc1n(c2=O)Cc2cc3cc(NC(=O)[C@H](C)NC(=O)[C@@H](NC(=O)CCCCCN4C(=O)C=CC4=O)C(C)C)c(F)cc3nc2-1] |
| DL: NHC- C-VA- Compound 140 | 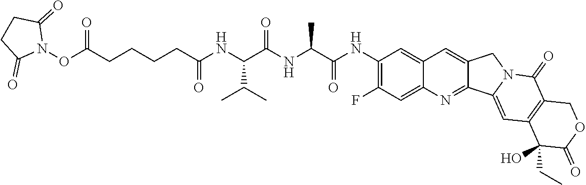 [SMILES: CC[C@@]1(O)C(=O)OCc2c1cc1n(c2=O)Cc2cc3cc(NC(=O)[C@H](C)NC(=O)[C@@H](NC(=O)CCCCC(=O)ON4C(=O)CCC4=O)C(C)C)c(F)cc3nc2-1] |
| DL: Azido- PEG8- VA- Compound 140 | 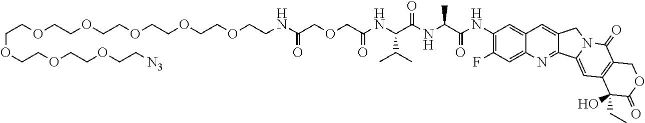 [SMILES: CC[C@@]1(O)C(=O)OCc2c1cc1n(c2=O)Cc2cc3cc(NC(=O)[C@H](C)NC(=O)[C@@H](NC(=O)COCC(=O)NCCOCCOCCOCCOCCOCCOCCOCCOCCN=[N+]=[N-])C(C)C)c(F)cc3nc2-1] |
| DL: MT- GGFG- Compound 140 | 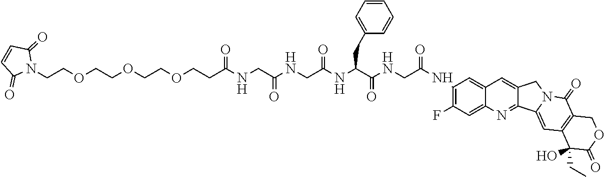 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCOCCOCCOCCN4C(=O)C=CC4=O)c(F)cc3nc2-1] |
| DL: MT- GGFG- Compound 141 | 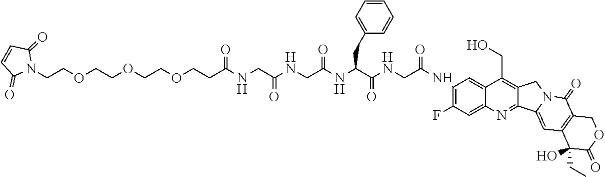 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CO] |
| DL: MT- GGFG- Compound 145 | 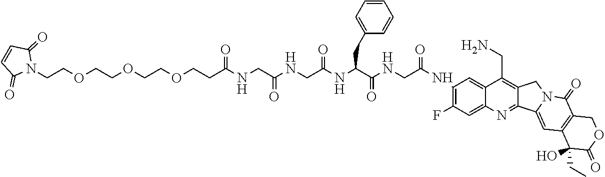 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN] |
| DL: MT- GGFG- Compound 148 | 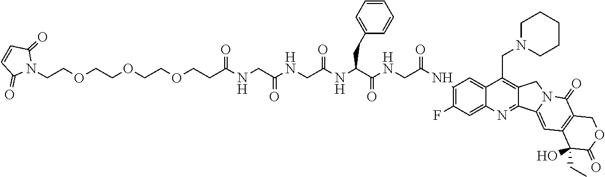 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCCCC1] |
| DL: MC- GGFG- Compound 140 | 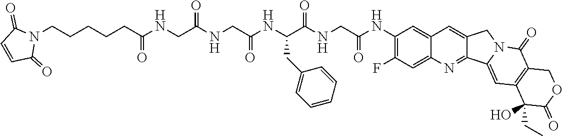 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCCCN4C(=O)C=CC4=O)c(F)cc3nc2-1] |
| DL: MT- GGFG- Compound 142 | 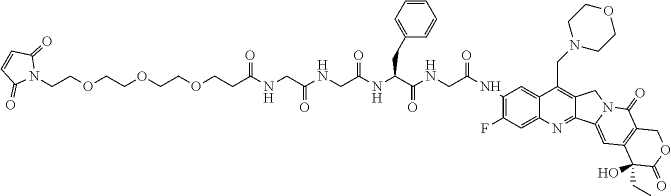 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCOCC1] |
| DL: MC- GGFG- Compound 141 | 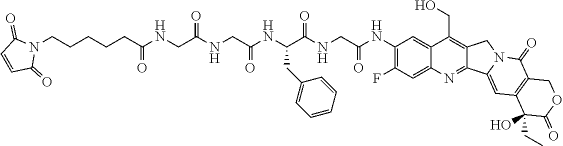 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCCCCN3C(=O)C=CC3=O)cc1c2CO] |
| DL: MC- VA- Compound 140 (DL: 201) | 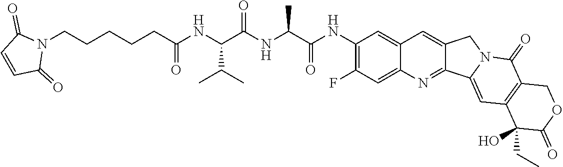 [SMILES: CC[C@@]1(O)C(=O)OCc2c1cc1n(c2=O)Cc2cc3cc(NC(=O)[C@H](C)NC(=O)[C@@H](NC(=O)CCCCCN4C(=O)C=CC4=O)C(C)C)c(F)cc3nc2-1] |
| DL: NHC- C-VA- Compound 140 | 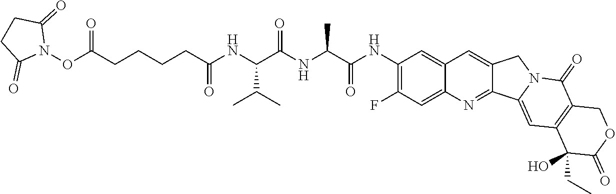 [SMILES: CC[C@@]1(O)C(=O)OCc2c1cc1n(c2=O)Cc2cc3cc(NC(=O)[C@H](C)NC(=O)[C@@H](NC(=O)CCCCC(=O)ON4C(=O)CCC4=O)C(C)C)c(F)cc3nc2-1] |
| DL: Azido- PEG8- VA- Compound 140 | 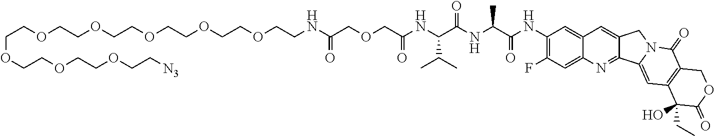 [SMILES: CC[C@@]1(O)C(=O)OCc2c1cc1n(c2=O)Cc2cc3cc(NC(=O)[C@H](C)NC(=O)[C@@H](NC(=O)COCC(=O)NCCOCCOCCOCCOCCOCCOCCOCCOCCN=[N+]=[N-])C(C)C)c(F)cc3nc2-1] |

TABLE 10
Exemplary drug-linker (DL) structures comprising camptothecin analogues of Formula (1) with either a C7 or C10 linkage

| Name | Structure |
|---|---|
| DL: 200 | 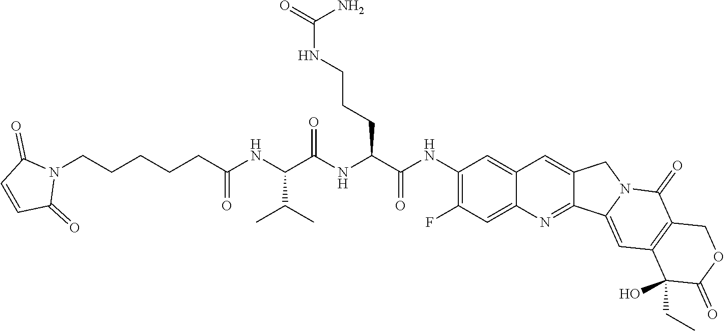 [SMILES: CC[C@@]1(O)C(=O)OCc2c1cc1n(c2=O)Cc2cc3cc(NC(=O)[C@H](CCCNC(N)=O)NC(=O)[C@@H](NC(=O)CCCCCN4C(=O)C=CC4=O)C(C)C)c(F)cc3nc2-1] |
| DL: 202 | 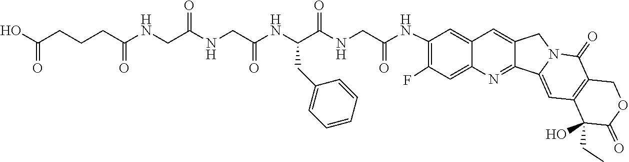 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCC(=O)O)c(F)cc3nc2-1] |
| DL: 203 | 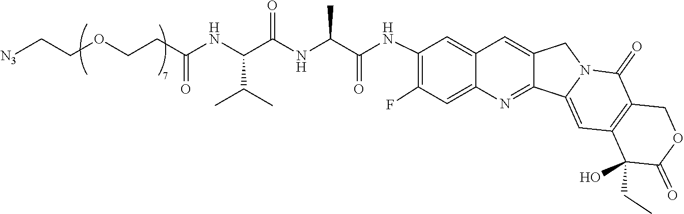 |
| DL: 204 | 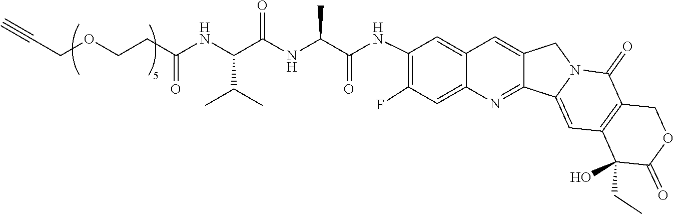 |
| DL: 205 | 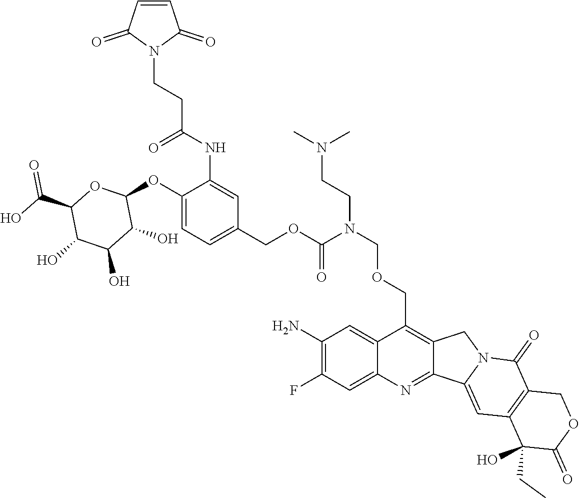 [SMILES: CC[C@@]1(O)C(=O)OCc2c1cc1n(c2=O)Cc2c-1nc1cc(F)c(N)cc1c2COCN(CCN(C)C)C(=O)OCc1ccc(O[C@@H]2O[C@H](C(=O)O)[C@@H](O)[C@H](O)[C@H]2O)c(NC(=O)CCN2C(=O)C=CC2=O)c1] |
| DL: 206 | 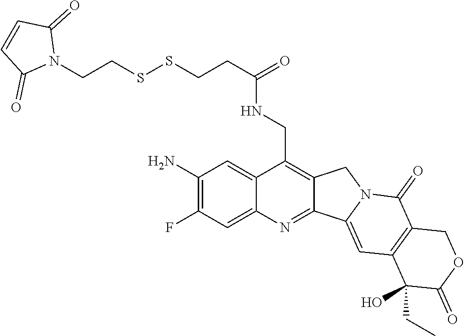 [SMILES: CC[C@@]1(O)C(=O)OCc2c1cc1n(c2=O)Cc2c-1nc1cc(F)c(N)cc1c2CNC(=O)CCSSCCN1C(=O)C=CC1=O] |
| DL: 207 | 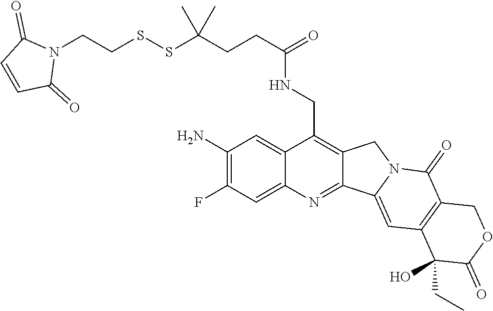 [SMILES: CC[C@@]1(O)C(=O)OCc2c1cc1n(c2=O)Cc2c-1nc1cc(F)c(N)cc1c2CNC(=O)CCC(C)(C)SSCCN1C(=O)C=CC1=O] |
| DL: 208 | 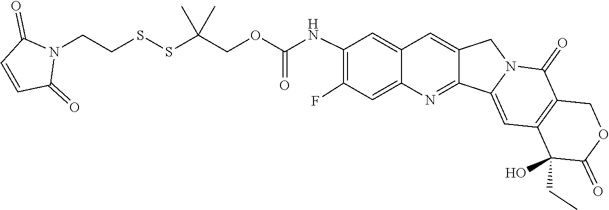 [SMILES: CC[C@@]1(O)C(=O)OCc2c1cc1n(c2=O)Cc2cc3cc(NC(=O)OCC(C)(C)SSCCN4C(=O)C=CC4=O)c(F)cc3nc2-1] |
| DL: 209 | 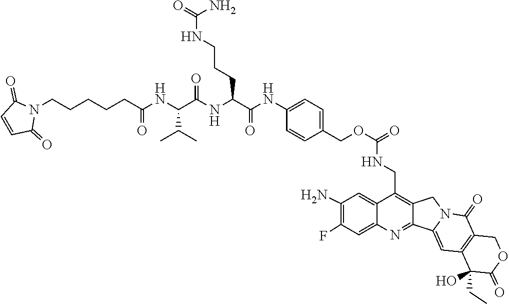 [SMILES: CC[C@@]1(O)C(=O)OCc2c1cc1n(c2=O)Cc2c-1nc1cc(F)c(N)cc1c2CNC(=O)OCc1ccc(NC(=O)[C@H](CCCNC(N)=O)NC(=O)[C@@H](NC(=O)CCCCCN2C(=O)C=CC2=O)C(C)C)cc1] |
| DL: 210 | 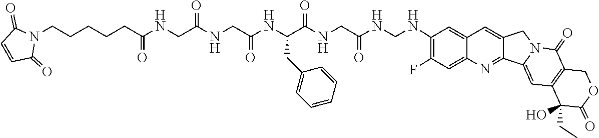 [SMILES: CC[C@@]1(O)C(=O)OCc2c1cc1n(c2=O)Cc2cc3cc(NCNC(=O)CNC(=O)[C@H](Cc4ccccc4)NC(=O)CNC(=O)CNC(=O)CCCCCN4C(=O)C=CC4=O)c(F)cc3nc2-1] |

TABLE 11
Exemplary conjugate (DC) structures comprising camptothecin analogues of Formula (I) with a C7 linkage

| Name | Structure |
|---|---|
| DC: MT- GGFG- Compound 104 | 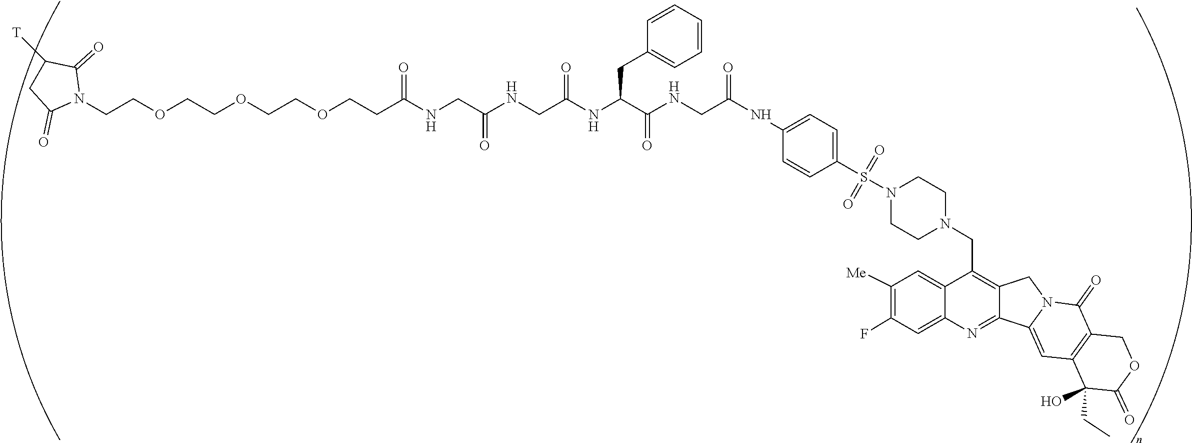 |
| DC: MT- GGFG- Compound 108 | 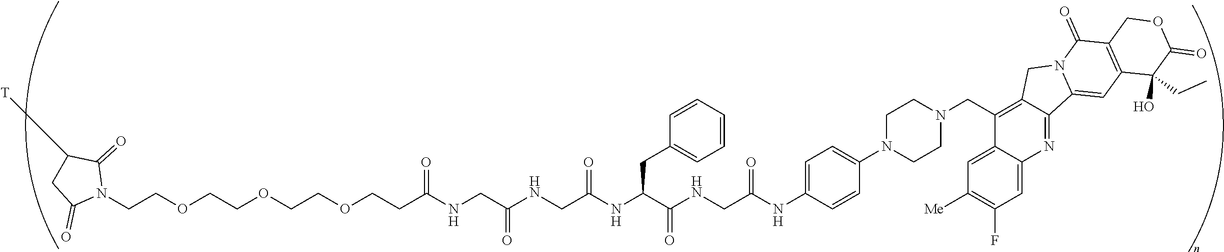 |
| DC: MT- GGFG- Compound 127 | 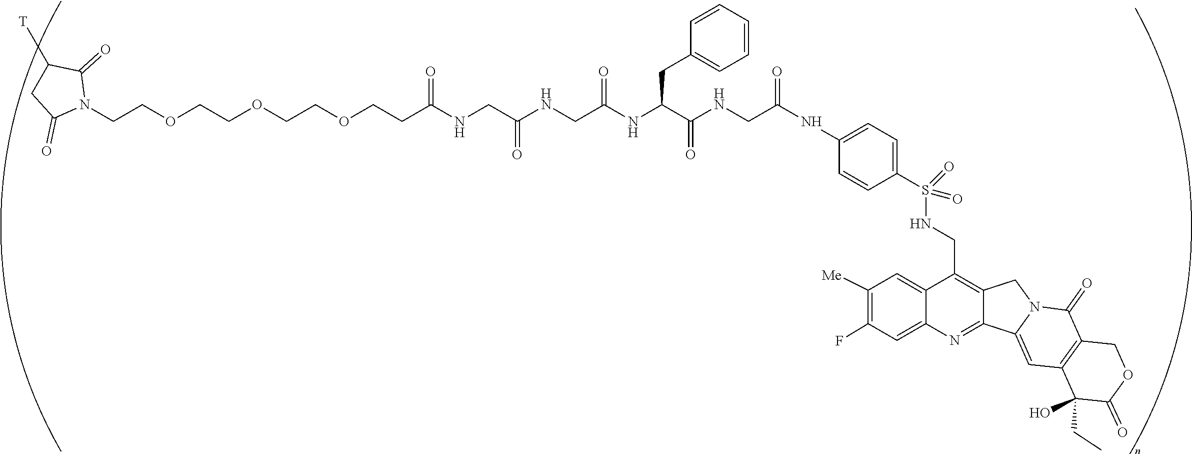 |
| DC: MT- GGFG- AM- Compound 136 | 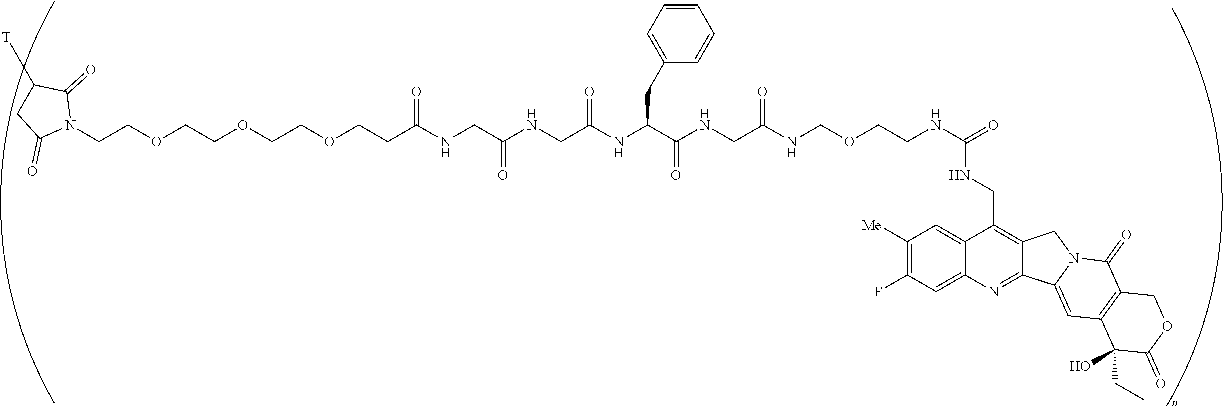 |
| DC: MT- GGFG- AM- Compound 139 | 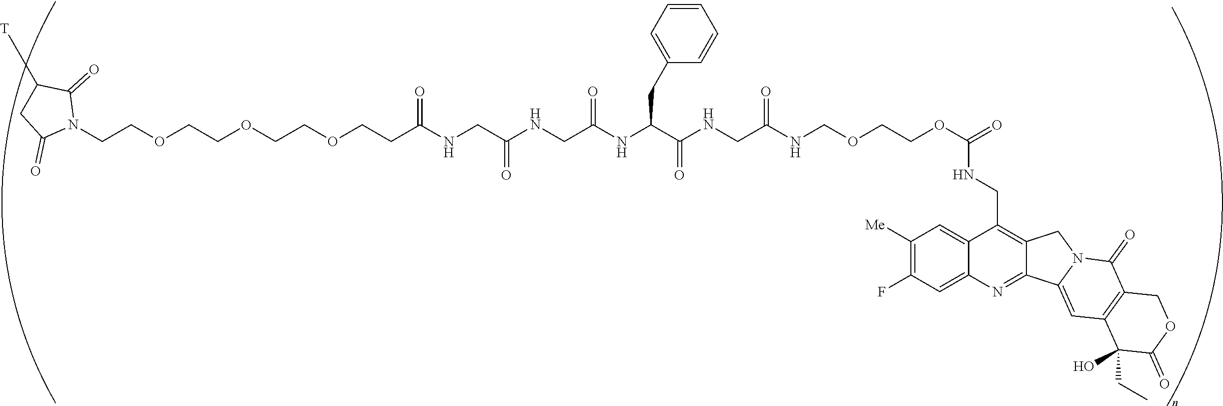 |
| DC: MT- GGFG- AM- Compound 129 | 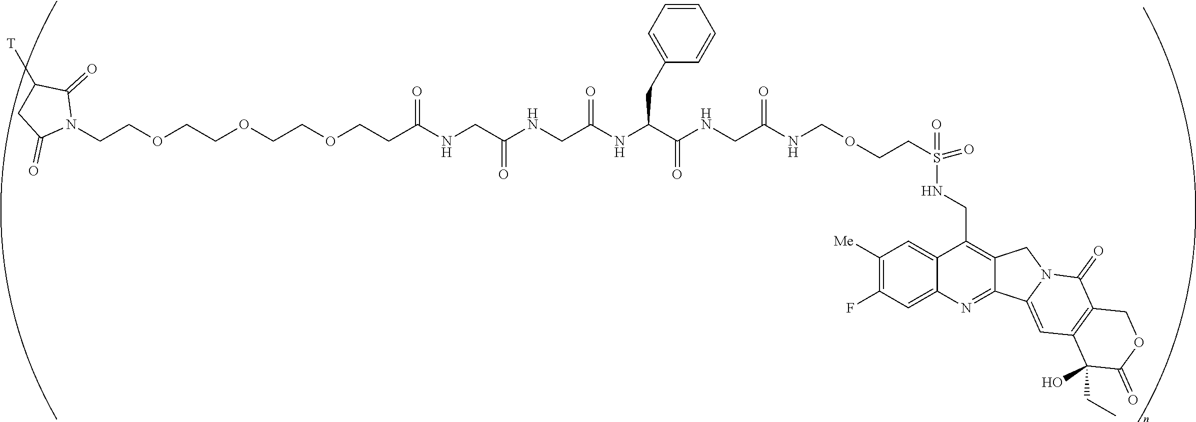 |
| DC: MT- GGFG- AM- Compound 113 | 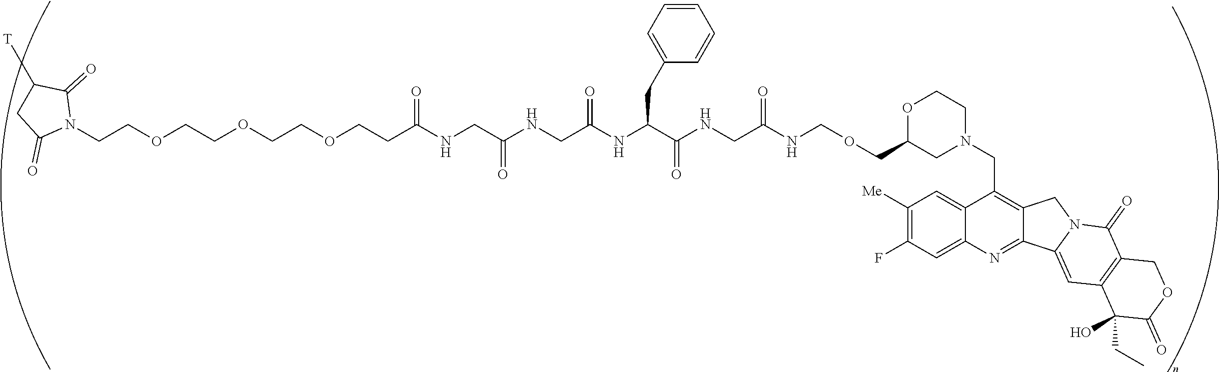 |
| DC: MT- GGFG- AM- Compound 141 | 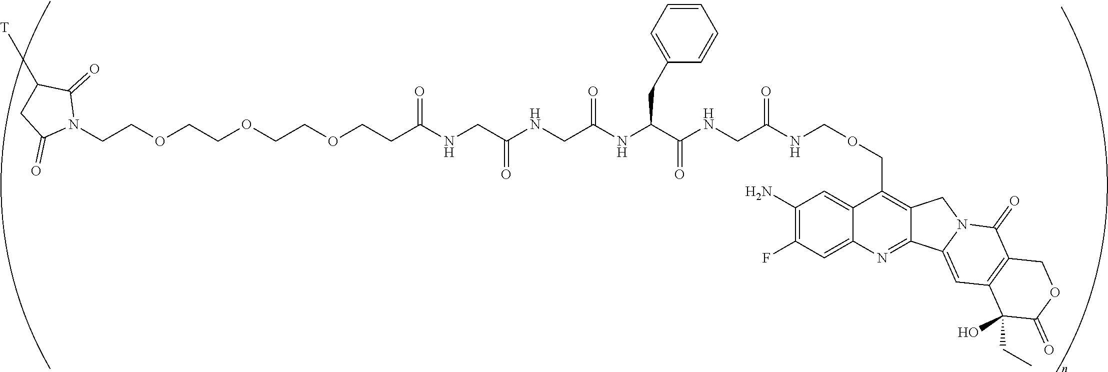 |
| DC: MC- GGFG- AM- Compound 141 | 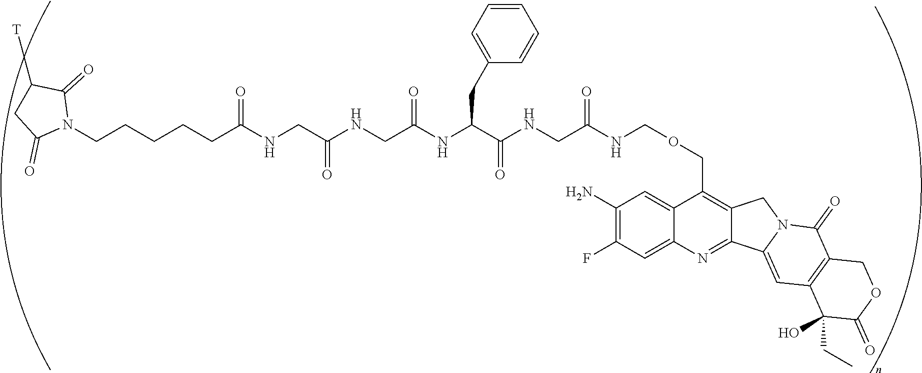 |
| DC: MT- GGFG- AM- Compound 117 | 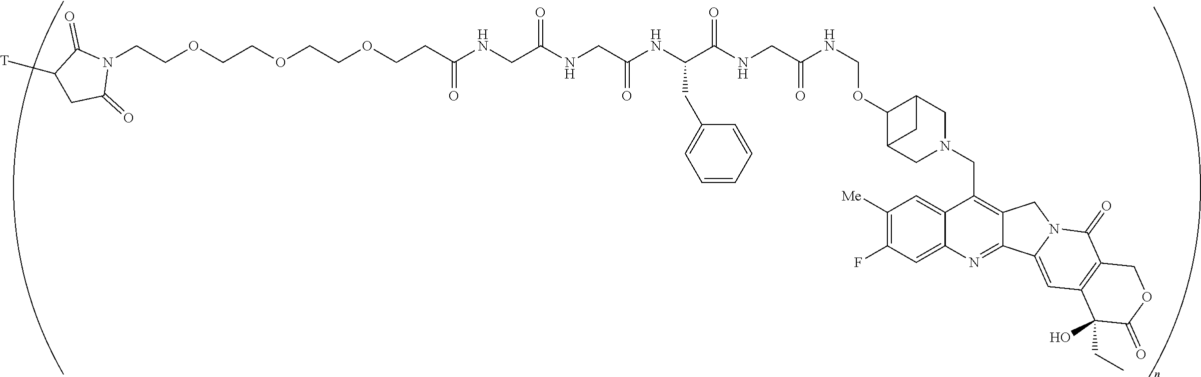 |
| DC: MT- GGFG- AM- Compound 118 | 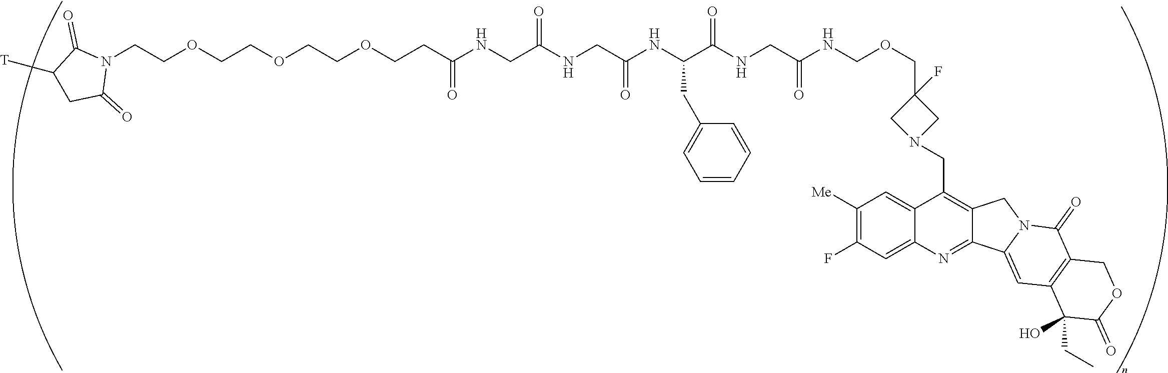 |
| DC: MC- GGFG- AM- Compound 139 | 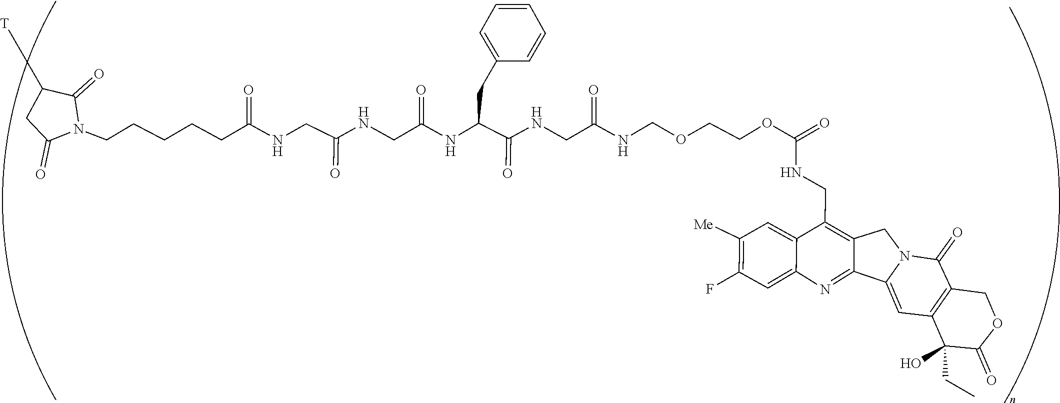 |
| DC: DiS- Compound 145 | 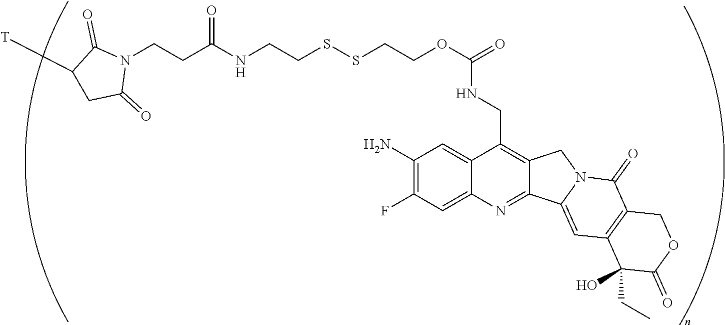 |

TABLE 12
Exemplary conjugate (DC) structures comprising camptothecin analogues of Formula (I) with a C10 linkage

| Name | Structure |
|---|---|
| DC: MT- GGFG- Compound 140 | 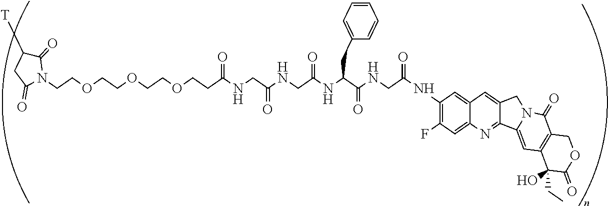 |
| DC: MT- GGFG- Compound 141 | 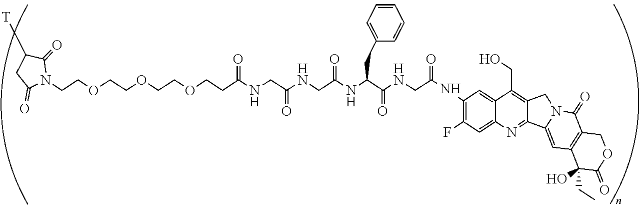 |
| DC: MT- GGFG- Compound 145 | 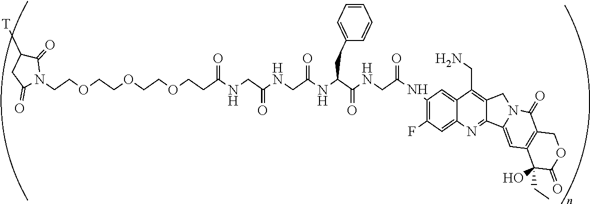 |
| DC: MT- GGFG- Compound 148 | 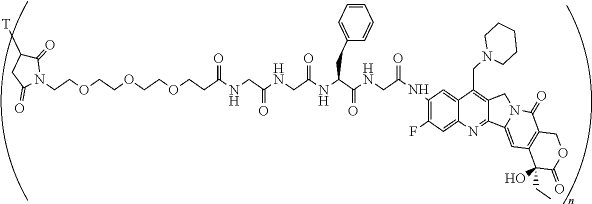 |
| DC: MT- GGFG- Compound 142 | 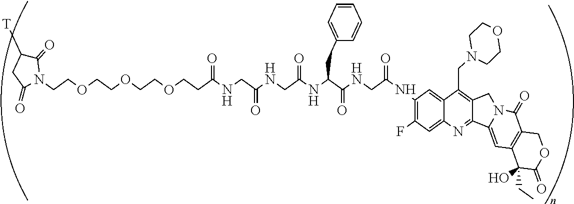 |
| DC: MC- GGFG- Compound 140 | 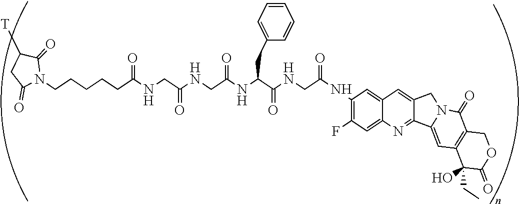 |
| DC: MC- GGFG- Compound 141 | 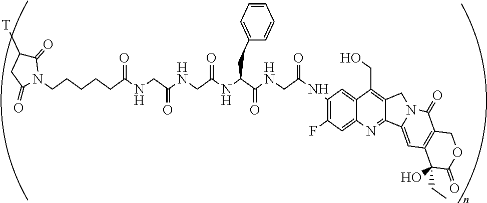 |
| DC: MC- VA- Compound 140 (DC: 301) | 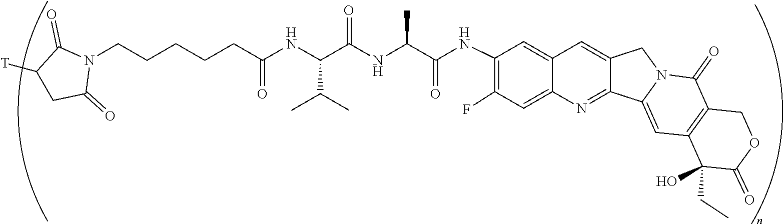 |
| DC: NHC- C-VA- Compound 140 | 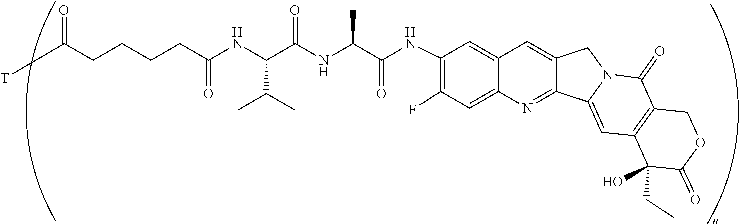 |
| DC: Azido- PEG8-VA- Compound 140 | 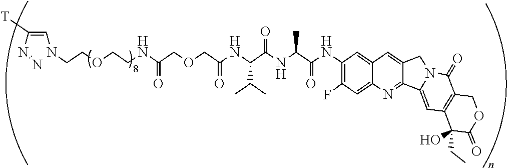 |

TABLE 13
Exemplary conjugate (DC) structures comprising camptothecin analogues of Formula (I) with either a C7 or C10 linkage

| Name | Structure |
|---|---|
| DC: 300 | 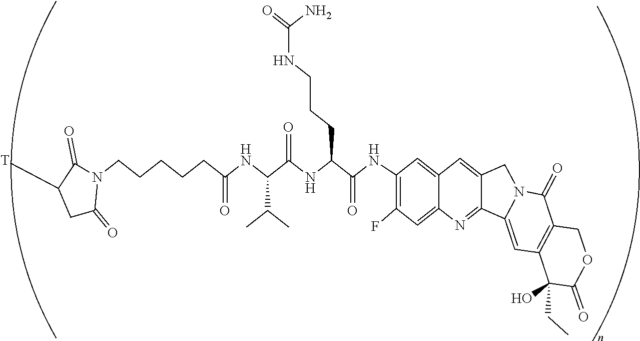 |
| DC: 302 | 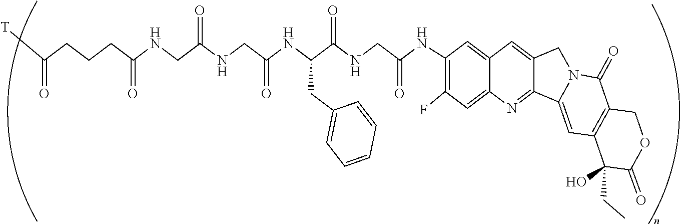 |
| DC: 303a | 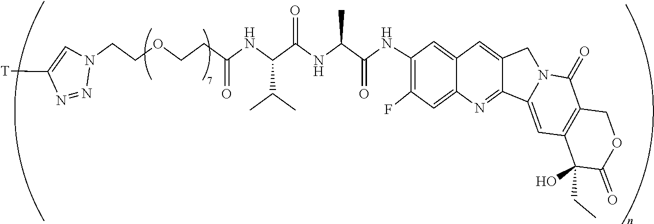 |
| DC: 303b | 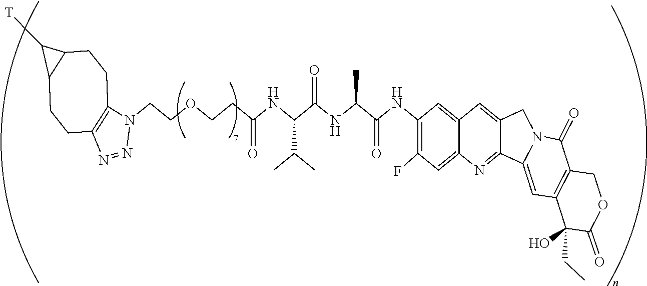 |
| DC: 304 | 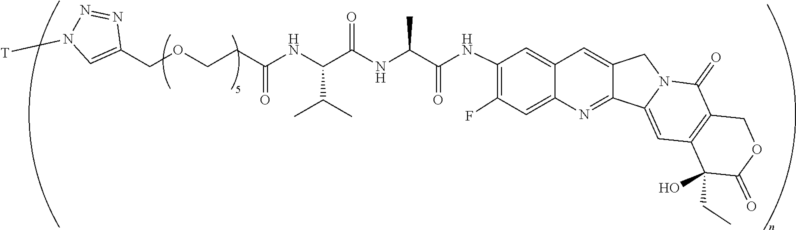 |
| DC: 305 | 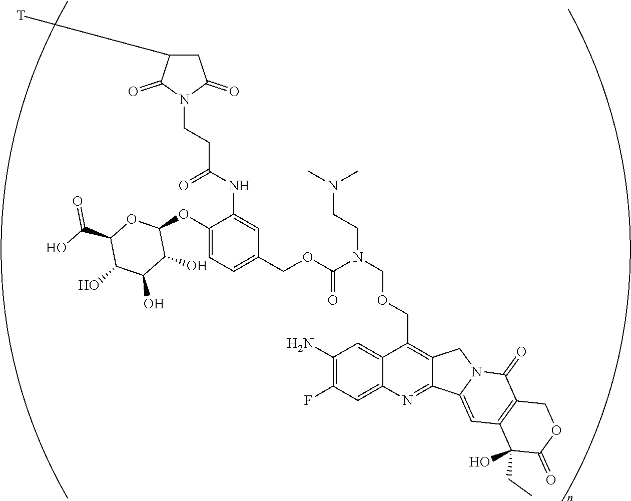 |
| DC: 306 | 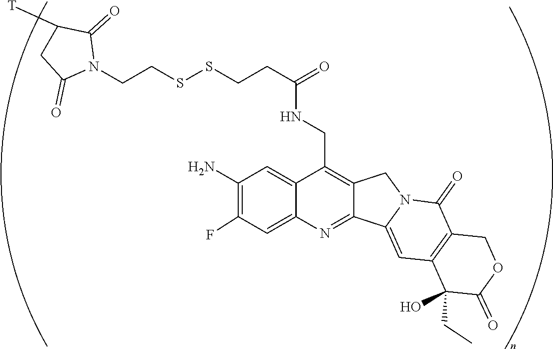 |
| DC: 307 | 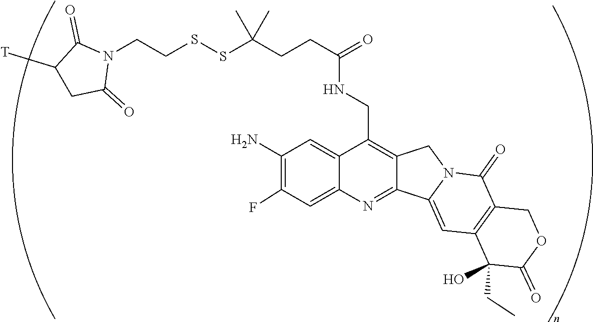 |
| DC: 308 | 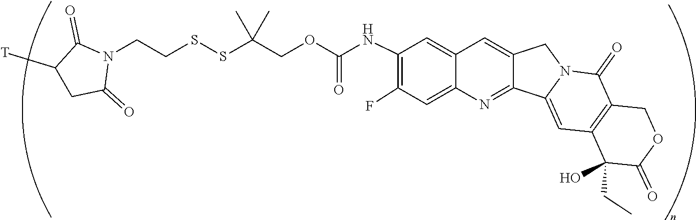 |
| DC: 309 | 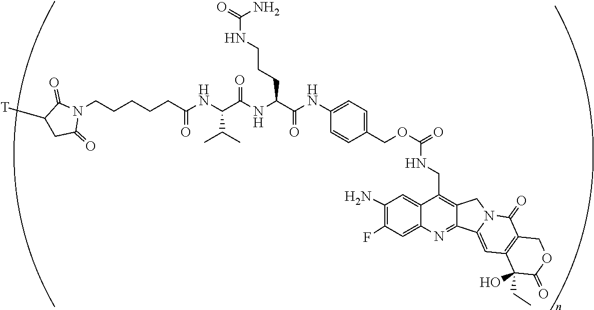 |
| DC: 310 | 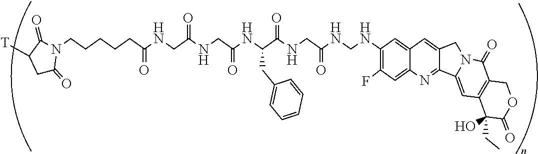 |

The following Examples are provided for illustrative purposes and are not intended to limit the scope of the invention in any way.

### CERTAIN EMBODIMENTS OF THE DISCLOSURE

Embodiment 1. An antibody-drug conjugate having the structure:

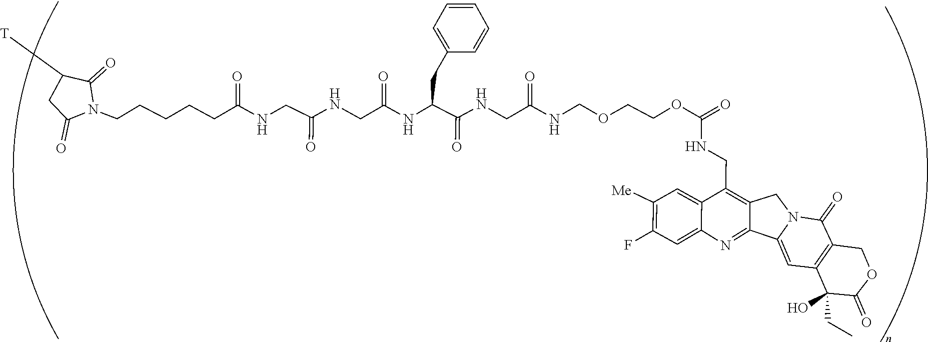

wherein n is between 2 and 6; and
T is an anti-NaPi2b (sodium-dependent phosphate transport protein 2B) antibody construct comprising an antigen-binding domain that binds to human NaPi2b, wherein the anti-NaPi2b antibody construct comprises two heavy chains comprising the sequence as set forth in SEQ ID NO:68, and two light chains comprising the sequence as set forth in SEQ ID NO:67, or wherein the anti-NaPi2b antibody construct comprises two heavy chains comprising the sequence as set forth in SEQ ID NO:66, and two light chains comprising the sequence as set forth in SEQ ID NO:67.

Embodiment 2. The antibody-drug conjugate according to embodiment 1, wherein n is between 3.5 and 4.5.

Embodiment 3. The antibody-drug conjugate according to embodiment 1, wherein n is about 4.

Embodiment 4. A pharmaceutical composition comprising an antibody-drug conjugate according to any one of embodiments 1 to 3, and a pharmaceutically acceptable carrier or diluent.

Embodiment 5. A method of inhibiting the proliferation of cancer cells comprising contacting the cells with an effective amount of the antibody-drug conjugate according to any one of embodiments 1 to 3.

Embodiment 6. A method of killing cancer cells comprising contacting the cells with an effective amount of the antibody-drug conjugate according to any one of embodiments 1 to 3.

Embodiment 7. A method of treating cancer in a subject in need thereof comprising administering to the subject an effective amount of the antibody-drug conjugate according to any one of embodiments 1 to 3.

Embodiment 8. Use of an effective amount of the antibody-drug conjugate according to any one of embodiments 1 to 3 for the treatment of cancer in a subject in need thereof.

Embodiment 9. An antibody-drug conjugate according to any one of embodiments 1 to 3 for use in therapy.

Embodiment 10. An antibody-drug conjugate according to any one of embodiments 1 to 3 for use in the treatment of cancer.

Embodiment 11. Use of an antibody-drug conjugate according to any one of embodiments 1 to 3 in the manufacture of a medicament for the treatment of cancer.

Embodiment 12. A kit comprising the antibody-drug conjugate according to any one of embodiments 1 to 3, and a label and/or package insert containing instructions for use.

### Examples

Examples 1-3 below illustrate various methods of preparing camptothecin analogues of Formula (I). It is understood that one skilled in the art may be able to make these compounds by similar methods or by combining other methods known in the art. It is also understood that one skilled in the art would be able to make, using the methods described below or similar methods, other compounds of Formula (I) not specifically illustrated below by using the appropriate starting components and modifying the parameters of the synthesis as needed. In general, starting components may be obtained from commercial sources such as Sigma Aldrich (Merck KGaA), Alfa Aesar and Maybridge (Thermo Fisher Scientific Inc.), Matrix Scientific, Tokyo Chemical Industry Ltd. (TCI) and Fluorochem Ltd., or synthesized according to sources known to those skilled in the art (see, for example, March's Advanced Organic Chemistry: Reactions, Mechanisms, and Structure, 7th edition, John Wiley & Sons, Inc., 2013) or prepared as described herein.

### Abbreviations

The following abbreviations are used throughout the Examples section: BCA: bicinchonic acid; Boc: di-tert-butyl dicarbonate; CE-SDS: capillary electrophoresis sodium dodecyl sulfate; DCM: dichloromethane; DTPA: diethylenetriamine pentaacetic acid; DIPEA: N,N-diisopropylethylamine; DMF: dimethylformamide; DMM™: (4-(4,6-dimethoxy-1,3,5-triazin-2-yl)-4-methyl-morpholinium chloride; EDC: 1-ethyl-3-(3-dimethylaminopropyl)carbodiimide; Fmoc: fluorenylmethyloxycarbonyl; HATU: hexafluorophosphate azabenzotriazole tetramethyl uronium; HIC: hydrophobic interaction chromatography; HOAt: 1-hydroxy-7-azabenzotriazole; HPLC: high-performance liquid chromatography; LC/MS: liquid chromatography mass spectrometry; MC: maleimidocaproyl; MT: maleimidotriethylene glycolate; NMM: N-methylmorpholine; PNP: p-nitrophenol; RP-UPLC-MS: reversed-phase ultra-high performance chromatography mass spectrometry; SEC: size exclusion chromatography; TCEP: tris(2-carboxyethyl) phosphine; Tfp: tetrafluorophenyl; TLC: thin layer chromatography; TFA: trifluoracetic acid.

### General Chemistry Procedures

### General Procedure 1: Conversion of Chloride to Amine

To a stirring solution of chloride compound in dimethylformamide (0.05-0.1 M) was added the appropriate secondary amine (3 eq.). Upon completion (determined by LC/MS, typically 1-3 h), the reaction mixture was purified by reverse-phase HPLC to provide the desired product after lyophilization.

### General Procedure 2: Conversion of Amine to Amide

To a stirring solution of amine compound in dimethylformamide (0.05-0.1 M) was added triethylamine (1.2 eq.), the appropriate carboxylic acid (1.1 eq.) followed by a solution of DMM™ (2 eq.) in water (1 M). Upon completion (determined by LC/MS, typically 16 h), the reaction mixture was purified by reverse-phase HPLC to provide the desired product after lyophilization.

### General Procedure 3: Conversion of Amine to Sulfonamide

To a stirring solution of amine compound in dimethylformamide (0.05-0.1 M) was added DIPEA (3 eq.) followed by the appropriate sulfonyl chloride. Upon completion (determined by LC/MS, typically 16 h), the reaction mixture was purified by reverse-phase HPLC to provide the desired product after lyophilization.

### General Procedure 4: 2-Step Conversion of Amine to Urea (Synthetic Scheme IV)

Step 1: To a stirring solution of amine compound in dichloromethane or dimethylformamide (0.05-0.1 M) was added p-nitrophenyl carbonate (1 eq.) then triethylamine (2 eq.). Upon completion (determined by LC/MS typically 1-4 h), the reaction mixture was concentrated to dryness then purified by reverse-phase HPLC to provide the desired PNP-carbamate intermediate after lyophilization. This intermediate can either used to generate a single analog or be divided into multiple batches in order to generate multiple analogs in the second step. Step 2: To the PNP-carbamate intermediate in dimethylformamide (0.1-0.2 M) was added the appropriate primary amine (3 eq.). Upon completion (determined by LC/MS, typically 1 h), the reaction mixture was purified by reverse-phase HPLC to provide the desired product after lyophilization.

### General Procedure 5: Conversion of Amine to Carbamate

To a stirring solution of amine compound in dichloromethane or dimethylformamide (0.05-0.1 M) was added p-nitrophenyl carbonate (1 eq.) then triethylamine (2 eq.). Upon completion (determined by LC/MS, typically 1-4 h), the appropriate alcohol was added to the resultant PNP-carbamate intermediate. Upon completion (determined by LC/MS, typically 1-16 h), the reaction mixture was purified by reverse-phase HPLC to provide the desired product after lyophilization.

### General Procedure 6: Removal of Boc Protecting Group

To a stirring solution of the Boc-protected amine compound in dichloromethane (0.1 M) was added TFA (20% by volume). Upon completion (determined by LC/MS, typically 1 h), the reaction mixture was concentrated in vacuo to provide a crude solid or was purified as described in General Procedure 9.

### General Procedure 7: Copper-Mediated Amide Coupling

To a rapidly stirring solution of Boc-GGFG-OH (3 eq.) and HOAt (3 eq.) in a 10% v/v mixture of dimethyl formamide in dichloromethane (0.02 M) was added EDC (HCl salt, 3 eq.). After 5 min, a solution of the amine containing payload (1 eq.) in a 10% v/v mixture of dimethyl formamide in dichloromethane (0.02 M) was added, followed immediately by the addition of CuCl₂ (4 eq.). Upon completion (determined by LC/MS, typically 1-16 h), the reaction mixture was concentrated in vacuo to provide a crude solid or was purified by preparative HPLC to provide the desired product after lyophilization.

### General Procedure 8: MT Installation

To a stirring solution of amine compound (1 eq.) in dimethylformamide (~0.02 M) was added a solution of MT-OTfp (1.2-1.5 eq.) in acetonitrile (~0.02 M) then DIPEA (10 μL, 4 eq.). Upon completion (determined by LC/MS, typically 1-16 h), the reaction mixture was concentrated in vacuo to provide a crude solid which was purified by preparative HPLC to provide the desired product after lyophilization.

### General Procedure 9: Compound Purification

Flash Chromatography: Crude reaction products were purified with Biotage® Snap Ultra columns (10, 25, 50, or 100 g) (Biotage, Charlotte, NC), eluting with linear gradients of ethyl acetate/hexanes or methanol/dichloromethane on a Biotage® Isolera™ automated flash system (Biotage, Charlotte, NC). Alternatively, reverse-phase flash purification was conducting using Biotage® Snap Ultra C18 columns (12, 30, 60, or 120 g), eluting with linear gradients of 0.1% TFA in acetonitrile/0.1% TFA in water. Purified compounds were isolated by either removal of organic solvents by rotavap or lyophilization of acetonitrile/water mixtures.

Preparative HPLC: Reverse-phase HPLC of crude compounds was performed using a Luna® 5-μm C18 100 Å (150×30 mm) column (Phenomenex, Torrance, CA) on an Agilent 1260 Infinity II preparative LC/MSD system (Agilent Technologies, Inc., Santa Clara, CA), and eluting with linear gradients of 0.1% TFA in acetonitrile/0.1% TFA in water. Purified compounds were isolated by lyophilization of acetonitrile/water mixtures.

### General Procedure 10: Compound Analysis

LC/MS: Reactions were monitored for completion and purified compounds were analyzed using a Kinetex® 2.6-μm C18 100 Å (30×3 mm) column (Phenomenex, Torrance, CA) on an Agilent 1290 HPLC/6120 single quad LC/MS system (Agilent Technologies, Inc., Santa Clara, CA), eluting with a 10 to 100% linear gradient of 0.1% formic acid in acetonitrile/0.1% formic acid in water.

NMR: ¹H NMR spectra were collected with a Bruker AVANCE III 300 Spectrometer (300 MHz) (Bruker Corporation, Billerica, MA). Chemical shifts are reported in parts per million (ppm).

### Example 1: Preparation of Camptothecin Analogues Having Methyl at the C10 Position

### 1.1: (S)-11-(chloromethyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 1.1)

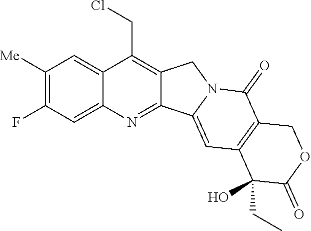 [SMILES: CC[C@@]1(O)C(=O)OCc2c1cc1n(c2=O)Cc2c-1nc1cc(F)c(C)cc1c2CCl]

The title compound was prepared according to the procedure provided in Li, et al., 2019, ACS Med. Chem. Lett., 10(10): 1386-1392.

### 1.2: (S)-11-(aminomethyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 1.2)

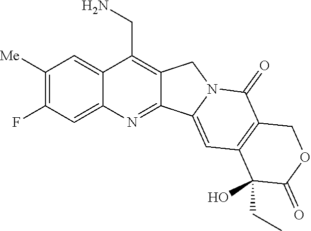 [SMILES: CC[C@@]1(O)C(=O)OCc2c1cc1n(c2=O)Cc2c-1nc1cc(F)c(C)cc1c2CN]

The title compound was prepared according to the procedure provided in Li, et al., 2019, ACS Med. Chem. Lett., 10(10): 1386-1392.

### 1.3: (S)-4-ethyl-8-fluoro-4-hydroxy-9-methyl-11-(morpholinomethyl)-1,12-dihydro-14H-pyrano[3′,4′:6,7]indolizino[1,2-b]quinoline-3,14(4H)-dione (Compound 100)

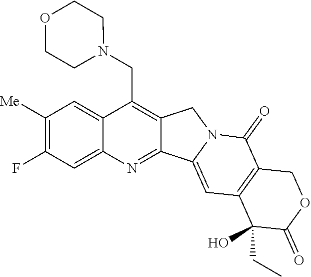 [SMILES: CC[C@@]1(O)C(=O)OCc2c1cc1n(c2=O)Cc2c-1nc1cc(F)c(C)cc1c2CN1CCOCC1]

The title compound was prepared according to General Procedure 1 starting from Compound 1.1 (10 mg) and morpholine. Preparative HPLC purification was accomplished as described in General Procedure 9, eluting with a 20 to 60% CH₃CN/H₂O+0.1% TFA gradient to give the title compound as an off-white solid (TFA salt, 3.6 mg, 26% yield).

LC/MS: Calc'd m/z=479.2 for C₂₆H₂₆FN₃O₅, found [M+H]⁺=480.4.

¹H NMR (300 MHz, CDCl₃) δ 8.20 (d, J=8.0 Hz, 1H), 7.82 (d, J=10.4 Hz, 1H), 7.67 (s, 1H), 5.77 (d, J=16.4 Hz, 1H), 5.42 (s, 2H), 5.33 (d, J=16.4 Hz, 1H), 4.26 (s, 2H), 3.81 (t, J=4.7 Hz, 4H), 2.82-2.76 (m, 4H), 2.57 (d, J=1.7 Hz, 3H), 1.99-1.82 (m, 2H), 1.06 (t, J=7.4 Hz, 3H).

### 1.4: (S)-4-ethyl-8-fluoro-4-hydroxy-9-methyl-11-((4-(phenylsulfonyl)piperazin-1-yl)methyl)-1,12-dihydro-14H-pyrano[3′,4′:6,7]indolizino[1,2-b]quinoline-3,14(4H)-dione (Compound 102)

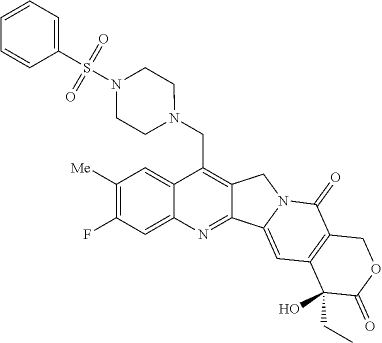 [SMILES: CC[C@@]1(O)C(=O)OCc2c1cc1n(c2=O)Cc2c-1nc1cc(F)c(C)cc1c2CN1CCN(S(=O)(=O)c2ccccc2)CC1]

The title compound was prepared according to General Procedure 1 starting from Compound 1.1 (10 mg) and 1-(phenylsulfonyl)piperazine. Preparative HPLC purification was accomplished as described in General Procedure 9, eluting with a 20 to 60% CH₃CN/H₂O+0.1% TFA gradient to give the title compound as an off-white solid (TFA salt, 3.6 mg, 21% yield).

LC/MS: Calc'd m/z=618.2 for C₃₂H₃₁FN₄O₆, found [M+H]⁺=619.4.

¹H NMR (300 MHz, CDCl₃) δ 8.07 (d, J=7.9 Hz, 1H), 7.88-7.44 (m, 7H), 5.73 (d, J=16.4 Hz, 1H), 5.33 (s, 2H), 5.33-5.26 (m, 1H), 4.19 (s, 2H), 3.12 (s, 4H), 2.80 (s, 4H), 2.54 (s, 3H), 1.90 (dt, J=11.6, 7.0 Hz, 2H), 1.04 (t, J=7.3 Hz, 3H).

### 1.5: (S)-11-((4-((4-aminophenyl)sulfonyl)piperazin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 104)

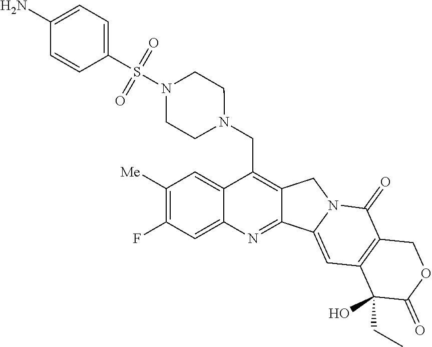 [SMILES: CC[C@@]1(O)C(=O)OCc2c1cc1n(c2=O)Cc2c-1nc1cc(F)c(C)cc1c2CN1CCN(S(=O)(=O)c2ccc(N)cc2)CC1]

The title compound was prepared according to General Procedure 1 starting from Compound 1.1 (10 mg) and 4-(piperazin-1-ylsulfonyl)aniline. Preparative HPLC purification was accomplished as described in General Procedure 9, eluting with a 20 to 60% CH₃CN/H₂O+0.1% TFA gradient to give the title compound as an off-white solid (TFA salt, 4.7 mg, 27% yield).

LC/MS: Calc'd m/z=633.2 for C₃₂H₃₂FN₅O₆, found [M+H]⁺=634.4.

¹H NMR (300 MHz, MeOD) δ 8.32 (d, J=8.0 Hz, 1H), 7.85 (d, J=10.5 Hz, 1H), 7.65 (s, 1H), 7.46 (d, J=8.7 Hz, 2H), 6.74 (d, J=8.7 Hz, 2H), 5.61 (d, J=16.5 Hz, 1H), 5.44 (s, 2H), 5.41 (d, J=16.5 Hz, 1H), 4.51 (s, 2H), 3.22-3.07 (m, 8H), 2.58 (s, 3H), 2.03-1.93 (m, 2H), 1.02 (t, J=7.3 Hz, 3H).

### 1.6: (S)-4-ethyl-8-fluoro-4-hydroxy-9-methyl-11-((4-methylpiperazin-1-yl)methyl)-1,12-dihydro-14H-pyrano[3′,4′:6,7]indolizino[1,2-b]quinoline-3,14(4H)-dione (Compound 106)

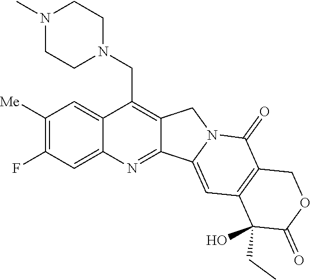 [SMILES: CC[C@@]1(O)C(=O)OCc2c1cc1n(c2=O)Cc2c-1nc1cc(F)c(C)cc1c2CN1CCN(C)CC1]

The title compound was prepared according to General Procedure 1 starting from Compound 1.1 (10 mg) and N-methylpiperazine. Preparative HPLC purification was accomplished as described in General Procedure 9, eluting with a 20 to 50% CH₃CN/H₂O+0.1% TFA gradient to give the title compound as an off-white solid (TFA salt, 3.6 mg, 25% yield).

LC/MS: Calc'd m/z=492.2 for C₂₇H₂₉FN₄O₄, found [M+H]⁺=493.4.

### 1.7. (S)-11-((4-(4-aminophenyl)piperazin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 108)

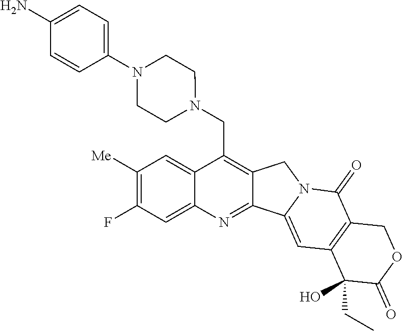 [SMILES: CC[C@@]1(O)C(=O)OCc2c1cc1n(c2=O)Cc2c-1nc1cc(F)c(C)cc1c2CN1CCN(c2ccc(N)cc2)CC1]

The title compound was prepared according to General Procedure 1 starting from Compound 1.1 (10 mg) and 4-(piperazin-1-yl)aniline. Preparative HPLC purification was accomplished as described in General Procedure 9, eluting with a 20 to 50% CH₃CN/H₂O+0.1% TFA gradient to give the title compound as an off-white solid (TFA salt, 3.7 mg, 23% yield).

LC/MS: Calc'd m/z=569.2 for C₃₂H₃₂FN₅O₄, found [M+H]⁺=570.4.

¹H NMR (300 MHz, MeOD) δ 8.39 (d, J=8.1 Hz, 1H), 7.79 (d, J=10.6 Hz, 1H), 7.21 (d, J=9.0 Hz, 2H), 7.14 (d, J=9.0 Hz, 2H), 5.62 (d, J=16.4 Hz, 1H), 5.49 (s, 2H), 5.41 (d, J= 16.4 Hz, 1H), 4.45 (s, 2H), 3.44-3.38 (m, 4H), 3.06-3.00 (m, 4H), 2.58 (d, J=1.8 Hz, 3H), 2.00-1.89 (m, 2H), 1.03 (t, J=7.3 Hz, 3H).

### 1.8: (S)-4-ethyl-8-fluoro-4-hydroxy-9-methyl-11-(piperidin-1-ylmethyl)-1,12-dihydro-14H-pyrano[3′,4′:6,7]indolizino[1,2-b]quinoline-3,14(4H)-dione (Compound 110)

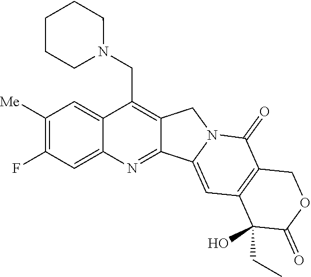 [SMILES: CC[C@@]1(O)C(=O)OCc2c1cc1n(c2=O)Cc2c-1nc1cc(F)c(C)cc1c2CN1CCCCC1]

The title compound was prepared according to General Procedure 1 starting from Compound 1.1 (10 mg) and piperidine. Preparative HPLC purification was accomplished as described in General Procedure 9, eluting with a 10 to 60% CH₃CN/H₂O+0.1% TFA gradient to give the title compound as an off-white solid (TFA salt, 1.5 mg, 11% yield).

LC/MS: Calc'd m/z=477.2 for C₂₇H₂₈FN₃O₄, found [M+H]⁺=478.2.

¹H NMR (300 MHz, MeOD) δ 8.34 (d, J=7.6 Hz, 1H), 7.94 (d, J=10.3 Hz, 1H), 7.70 (s, 1H), 5.63 (d, J=16.4 Hz, 1H), 5.52 (s, 2H), 5.44 (d, J=16.5 Hz, 1H), 4.99 (s, 2H), 3.73-3.46 (m, 4H), 2.64 (s, 3H), 2.03-1.90 (m, 2H), 1.90-1.84 (m, 6H), 1.03 (t, J=7.4 Hz, 3H).

### 1.9: tert-butyl (S)-4-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazine-1-carboxylate (Compound 111)

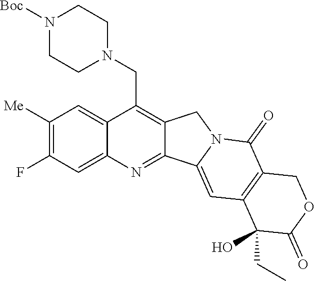 [SMILES: CC[C@@]1(O)C(=O)OCc2c1cc1n(c2=O)Cc2c-1nc1cc(F)c(C)cc1c2CN1CCN(C(=O)OC(C)(C)C)CC1]

The title compound was prepared according to General Procedure 1 starting from Compound 1.1 (10 mg) and tert-butyl piperazine-1-carboxylate. Preparative HPLC purification was accomplished as described in General Procedure 9, eluting with a 10 to 60% CH₃CN/H₂O+0.1% TFA gradient to give the title compound as an off-white solid (TFA salt, 6.6 mg, 40% yield).

LC/MS: Calc'd m/z=578.2 for C₃₁H₃₅FN₄O₆, found [M+H]⁺=579.4.

### 1.10: (S)-4-ethyl-8-fluoro-4-hydroxy-9-methyl-11-(piperazin-1-ylmethyl)-1,12-dihydro-14H-pyrano[3′,4′:6,7]indolizino[1,2-b]quinoline-3,14(4H)-dione (Compound 112)

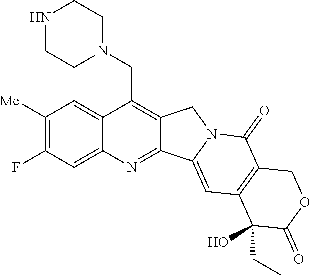 [SMILES: CC[C@@]1(O)C(=O)OCc2c1cc1n(c2=O)Cc2c-1nc1cc(F)c(C)cc1c2CN1CCNCC1]

The title compound was prepared according to General Procedure 6 starting from Compound 111 (5.0 mg) to give the title compound as an off-white solid (TFA salt, 4.4 mg).

LC/MS: Calc'd m/z=478.2 for C₂₆H₂₇FN₄O₄, found [M+H]⁺=479.2.

### 1.11: (S)-4-ethyl-8-fluoro-4-hydroxy-11-(((R)-2-(hydroxymethyl)morpholino)methyl)-9-methyl-1,12-dihydro-14H-pyrano[3′,4′:6,7]indolizino[1,2-b]quinoline-3,14(4H)-dione (Compound 113)

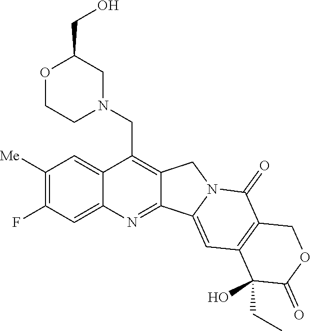 [SMILES: CC[C@@]1(O)C(=O)OCc2c1cc1n(c2=O)Cc2c-1nc1cc(F)c(C)cc1c2CN1CCO[C@@H](CO)C1]

The title compound was prepared according to General Procedure 1 starting from Compound 1.1 (10 mg) and (R)-morpholin-2-yl methanol. Preparative HPLC purification was accomplished as described in General Procedure 9, eluting with a 10 to 60% CH₃CN/H₂O+0.1% TFA gradient to give the title compound as an off-white solid (TFA salt, 4.6 mg, 32% yield).

LC/MS: Calc'd m/z=509.2 for C₂₇H₂₈FN₃O₆, found [M+H]⁺=510.4.

### 1.12: (4S)-4-ethyl-8-fluoro-4-hydroxy-11-((3-(hydroxymethyl)thiomorpholino)methyl)-9-methyl-1,12-dihydro-14H-pyrano[3′,4′:6,7]indolizino[1,2-b]quinoline-3,14(4H)-dione (Compound 114)

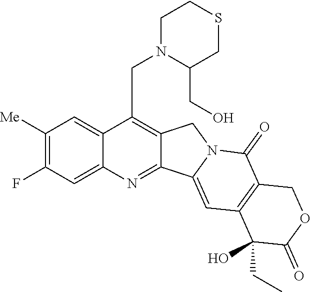 [SMILES: CC[C@@]1(O)C(=O)OCc2c1cc1n(c2=O)Cc2c-1nc1cc(F)c(C)cc1c2CN1CCSCC1CO]

The title compound was prepared according to General Procedure 1 starting from Compound 1.1 (10 mg) and thiomorpholin-3-ylmethanol. Preparative HPLC purification was accomplished as described in General Procedure 9, eluting with a 10 to 60% CH₃CN/H₂O+0.1% TFA gradient to give the title compound as an off-white solid (TFA salt, 1.5 mg, 12% yield).

LC/MS: Calc'd m/z=525.6 for C₂₇H₂₈FN₃O₅S, found [M+H]⁺=526.5.

¹H NMR (300 MHz, 10% D₂O/CD₃CN) 8.36 (d, J=8.1 Hz, 1H), 7.83 (d, J=10.7 Hz, 1H), 7.50 (s, 1H), 5.57 (d, J=16.4 Hz, 1H), 5.52-5.29 (m, 3H), 5.02 (d, J=14.6 Hz, 1H), 4.71-4.54 (m, 1H), 4.27 (dd, J=12.4, 5.0 Hz, 1H), 3.98 (dd, J=12.3, 3.4 Hz, 1H), 3.55 (s, 1H), 3.30-3.03 (m, 4H) 2.97-2.72 (m, 3H), 2.62 (s, 1H), 2.55 (s, 3H), 0.95 (t, J=7.4 Hz, 3H).

### 1.13: (4S)-4-ethyl-8-fluoro-4-hydroxy-11-((4-(hydroxymethyl)-2-oxa-5-azabicyclo[2.2.1] heptan-5-yl)methyl)-9-methyl-1,12-dihydro-14H-pyrano[3′,4′:6,7]indolizino[1,2-b]quinoline-3,14(4H)-dione (Compound 115)

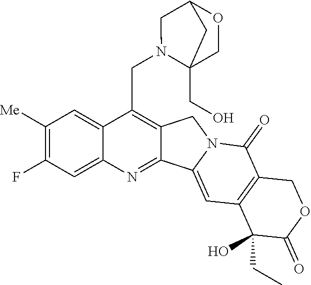 [SMILES: CC[C@@]1(O)C(=O)OCc2c1cc1n(c2=O)Cc2c-1nc1cc(F)c(C)cc1c2CN1CC2CC1(CO)CO2]

The title compound was prepared according to General Procedure 1 starting from Compound 1.1 (10 mg) and 2-oxa-5-azabicyclo[2.2.1]heptan-4-yl methanol. Preparative HPLC purification was accomplished as described in General Procedure 9, eluting with a 10 to 60% CH₃CN/H₂O+0.1% TFA gradient to give the title compound as an off-white solid (TFA salt, 3.5 mg, 29% yield).

LC/MS: Calc'd m/z=521.5 for C₂₈H₂₈FN₃O₆, found [M+H]⁺=522.5.

¹H NMR (300 MHz, 10% D₂O/CD₃CN) δ 8.36 (d, J=7.9 Hz, 1H), 7.86 (dd, J=10.6, 5.0 Hz, 1H), 7.50 (d, J=1.8 Hz, 1H), 5.63-5.49 (m, 2H), 5.37 (dd, J=17.8, 14.1 Hz, 2H), 5.05 (s, 2H), 4.63 (d, J=2.5 Hz, 1H), 4.55 (d, J=10.7 Hz, 1H), 4.33 (s, 2H), 3.92 (d, J=10.7 Hz, 1H), 3.36 (s, 2H), 2.57 (s, 3H), 2.41-2.13 (m, 2H), 1.97-1.85 (m, 2H), 0.95 (t, J=7.4 Hz, 3H).

### 1.14: (4S)-4-ethyl-8-fluoro-4-hydroxy-11-((3-(hydroxymethyl)-1,1-dioxidothiomorpholino) methyl)-9-methyl-1,12-dihydro-14H-pyrano[3′,4′:6,7]indolizino[1,2-b]quinoline-3,14(4H)-dione (Compound 116)

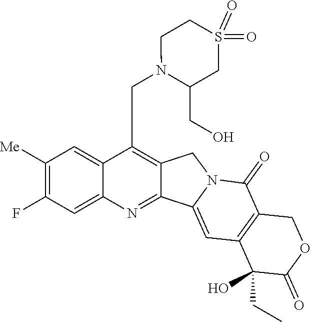 [SMILES: CC[C@@]1(O)C(=O)OCc2c1cc1n(c2=O)Cc2c-1nc1cc(F)c(C)cc1c2CN1CCS(=O)(=O)CC1CO]

The title compound was prepared according to General Procedure 1 starting from Compound 1.1 (10 mg) and 3-(hydroxymethyl)-1λ⁶-thiomorpholine-1,1-dione. Purification was accomplished as described in General Procedure 9, eluting with a 10 to 60% CH₃CN/H₂O+0.1% TFA gradient to give the title compound as an off-white solid (TFA salt, 0.2 mg, 2% yield).

LC/MS: Calc'd m/z=557.6 for C₂₇SFN₃O₇S, found [M+H]⁺=558.4.

¹H NMR (300 MHz, 10% D₂O/CD₃CN) δ 8.44 (d, J=8.2 Hz, 1H), 7.80 (d, J=11.0 Hz, 1H), 7.50 (s, 1H), 5.58 (d, J=16.5 Hz, 1H), 5.45-5.26 (m, 3H), 4.60 (d, J=14.9 Hz, 1H), 4.33 (d, J=14.7 Hz, 1H), 3.88 (d, J=4.8 Hz, 2H), 3.41-2.85 (m, 4H), 2.53 (s, 2H), 2.19 (p, J=2.5 Hz, 2H), 1.74 (p, J=2.5 Hz, 2H), 1.27 (s, 2H), 0.95 (t, J=7.4 Hz, 3H).

### 1.15: (4S)-4-ethyl-8-fluoro-4-hydroxy-11-((6-hydroxy-3-azabicyclo[3.1.1]heptan-3-yl)methyl)-9-methyl-1,12-dihydro-14H-pyrano[3′,4′:6,7]indolizino[1,2-b]quinoline-3,14(4H)-dione (Compound 117)

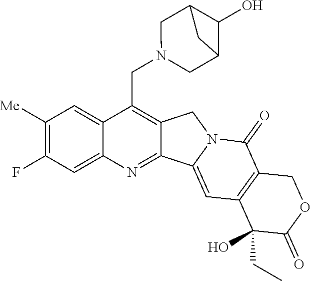 [SMILES: CC[C@@]1(O)C(=O)OCc2c1cc1n(c2=O)Cc2c-1nc1cc(F)c(C)cc1c2CN1CC2CC(C1)C2O]

The title compound was prepared according to General Procedure 1 starting from Compound 1.1 (10 mg) and 3-azabicyclo[3.1.1]heptan-6-ol. Preparative HPLC purification was accomplished as described in General Procedure 9, eluting with a 10 to 60% CH₃CN/H₂O+0.1% TFA gradient to give the title compound as an off-white solid (TFA salt, 1.3 mg, 11% yield).

LC/MS: Calc'd m/z=505.5 for C₂₈H₂₈FN₃O₅, found [M+H]⁺=506.6.

¹H NMR (300 MHz, 10% D₂O/CD₃CN) δ 8.25 (d, J=7.9 Hz, 1H), 7.87 (d, J=10.6 Hz, 1H), 7.50 (s, 1H), 5.65-5.27 (m, 4H), 4.98 (s, 2H), 4.24 (s, 1H), 3.83-3.57 (m, 4H), 2.54 (s, 5H), 2.01-1.86 (m, 2H), 1.70 (s, 2H), 0.95 (t, J=7.3 Hz, 3H).

### 1.16: (S)-4-ethyl-8-fluoro-11-((3-fluoro-3-(hydroxymethyl)azetidin-1-yl)methyl)-4-hydroxy-9-methyl-1,12-dihydro-14H-pyrano[3′,4′:6,7]indolizino[1,2-b]quinoline-3,14(4H)-dione (Compound 118)

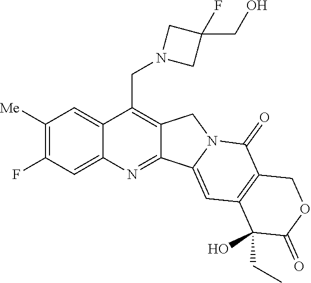 [SMILES: CC[C@@]1(O)C(=O)OCc2c1cc1n(c2=O)Cc2c-1nc1cc(F)c(C)cc1c2CN1CC(F)(CO)C1]

The title compound was prepared according to General Procedure 1 starting from Compound 1.1 (10 mg) and 3-fluoroazetidin-3-yl methanol. Preparative HPLC purification was accomplished as described in General Procedure 9, eluting with a 10 to 60% CH₃CN/H₂O+0.1% TFA gradient to give the title compound as an off-white solid (TFA salt, 1.4 mg, 12% yield).

LC/MS: Calc'd m/z=497.5 for C₂₆H₂₅F₂N₃O₅, found [M+H]⁺=498.4.

¹H NMR (300 MHz, 10% D₂O/CD₃CN) δ 8.24 (d, J=7.9 Hz, 1H), 7.85 (d, J=10.7 Hz, 1H), 7.50 (s, 1H), 5.57 (d, J=16.5 Hz, 1H), 5.48-5.28 (m, 3H), 4.98 (s, 2H), 4.44-4.14 (m, 4H), 3.78 (d, J=14.9 Hz, 2H), 2.01-1.86 (m, 2H), 0.95 (t, J=7.4 Hz, 3H).

### 1.17. (S)-4-ethyl-8-fluoro-4-hydroxy-11-((3-(hydroxymethyl)azetidin-1-yl)methyl)-9-methyl-1,12-dihydro-14H-pyrano[3′,4′:6,7]indolizino[1,2-b]quinoline-3,14(4H)-dione (Compound 119)

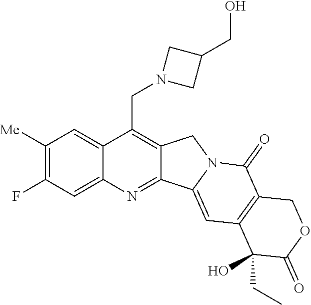 [SMILES: CC[C@@]1(O)C(=O)OCc2c1cc1n(c2=O)Cc2c-1nc1cc(F)c(C)cc1c2CN1CC(CO)C1]

The title compound was prepared according to General Procedure 1 starting from Compound 1.1 (10 mg) and azetidin-3-ylmethanol. Preparative HPLC purification was accomplished as described in General Procedure 9, eluting with a 10 to 60% CH₃CN/H₂O+0.1% TFA gradient to give the title compound as an off-white solid (TFA salt, 0.5 mg, 4.5% yield).

LC/MS: Calc'd m/z=479.5 for C₂₆H₂₆FN₃O₅, found [M+H]⁺=480.4.

¹H NMR (300 MHz, 10% D₂O/CD₃CN) δ 8.23 (d, J=7.8 Hz, 1H), 7.90 (d, J=10.6 Hz, 1H), 7.53 (s, 1H), 5.58 (d, J=16.5 Hz, 1H), 5.50-5.28 (m, 3H), 5.01 (s, 2H), 4.31-4.17 (m, 2H), 4.15-4.00 (m, 2H), 3.62 (d, J=3.9 Hz, 2H), 2.58 (s, 3H), 2.01-1.86 (m, 2H), 0.96 (t, J=7.4 Hz, 3H).

### 1.18: (4S)-11-((4,4-difluoro-3-(hydroxymethyl)piperidin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 120)

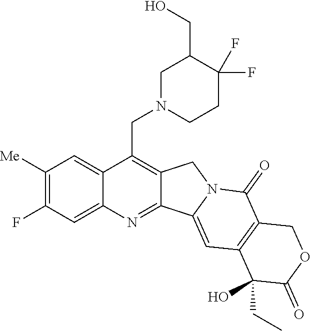 [SMILES: CC[C@@]1(O)C(=O)OCc2c1cc1n(c2=O)Cc2c-1nc1cc(F)c(C)cc1c2CN1CCC(F)(F)C(CO)C1]

The title compound was prepared according to General Procedure 1 starting from Compound 1.1 (10 mg) and 4,4-difluoropiperidin-3-yl methanol. Preparative HPLC purification was accomplished as described in General Procedure 9, eluting with a 10 to 60% CH₃CN/H₂O+0.1% TFA gradient to give the title compound as an off-white solid (TFA salt, 4 mg, 32% yield).

LC/MS: Calc'd m/z=543.5 for C₂₈H₂₈F₃N₃O₅, found [M+H]⁺=544.4.

¹H NMR (300 MHz, 10% D₂O/CD₃CN) δ 8.25 (d, J=8.0 Hz, 1H), 7.77 (dd, J=10.7, 1.4 Hz, 1H), 7.47 (s, 1H), 5.55 (d, J=16.5 Hz, 1H), 5.42-5.25 (m, 3H), 4.66 (d, J=3.2 Hz, 2H), 3.90-3.77 (m, 1H), 3.71-3.45 (m, 4H), 2.24 (q, J=11.8, 9.2 Hz, 2H), 2.01-1.86 (m, 2H), 0.94 (t, J=7.4 Hz, 3H).

### 1.19: (S)-4-ethyl-8-fluoro-4-hydroxy-11-((1-(hydroxymethyl)-7-azabicyclo[2.2.1]heptan-7-yl)methyl)-9-methyl-1,12-dihydro-14H-pyrano[3′,4′:6,7]indolizino[1,2-b]quinoline-3,14(4H)-dione (Compound 121)

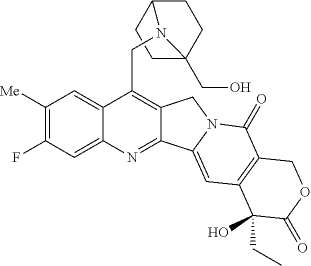 [SMILES: CC[C@@]1(O)C(=O)OCc2c1cc1n(c2=O)Cc2c-1nc1cc(F)c(C)cc1c2CN1C2CCC1(CO)CC2]

The title compound was prepare according to General 1 procedure 1 starting from Compound 1.1 (10 mg) and 7-azabicyclo[2.2.1]heptan-1-ylmethanol. Preparative HPLC purification was accomplished as described in General Procedure 9, eluting with a 10 to 60% CH₃CN/H₂O+0.1% TFA gradient to give the title compound as an off-white solid (TFA salt, 0.8 mg, 6.6% yield).

LC/MS: Calc'd m/z=519.6 for C₂₉H₃₀FN₃O₅, found [M+H]⁺=520.4.

¹H NMR (300 MHz, 10% D₂O/CD₃CN) δ 8.22 (s, 1H), 7.92 (d, J=10.7 Hz, 1H), 7.54 (s, 1H), 5.59 (dd, J=17.6, 7.6 Hz, 2H), 5.33 (t, J=17.4 Hz, 2H), 4.98-4.81 (m, 1H), 4.67-4.44 (m, 2H), 4.28-3.93 (m, 4H), 2.73 (s, 2H), 2.34-2.03 (m, 4H), 1.91 (d, J=14.0 Hz, 5H), 0.96 (t, J=7.4 Hz, 3H).

### 1.20: (S)—N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)methanesulfonamide (Compound 122)

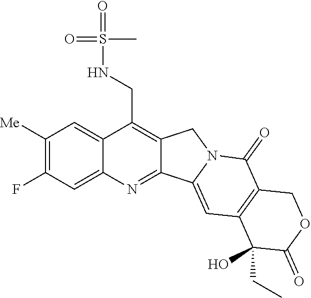 [SMILES: CC[C@@]1(O)C(=O)OCc2c1cc1n(c2=O)Cc2c-1nc1cc(F)c(C)cc1c2CNS(C)(=O)=O]

The title compound was prepared according to General Procedure 3 starting from Compound 1.2 (10 mg) and methane sulfonyl chloride. Preparative HPLC purification was accomplished as described in General Procedure 9, eluting with a 10 to 50% CH₃CN/H₂O+0.1% TFA gradient to give the title compound as an off-white solid (0.8 mg, 7% yield).

LC/MS: Calc'd m/z=487.1 for C₂₃H₂₂FN₃O₆S, found [M+H]⁺=488.2.

¹H NMR (300 MHz, MeOD) δ 8.33 (d, J=8.1 Hz, 1H), 7.83 (d, J=10.8 Hz, 1H), 7.68 (s, 1H), 5.62 (d, J=16.3 Hz, 1H), 5.52 (s, 2H), 5.42 (d, J=16.4 Hz, 1H), 4.87 (s, 2H), 3.06 (s, 3H), 2.59 (s, 3H), 2.06-1.93 (m, 2H), 1.03 (t, J=7.4 Hz, 3H).

### 1.21: (S)—N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-1-(4-nitrophenyl) methanesulfonamide (Compound 124)

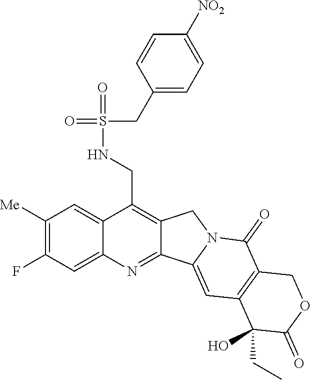 [SMILES: CC[C@@]1(O)C(=O)OCc2c1cc1n(c2=O)Cc2c-1nc1cc(F)c(C)cc1c2CNS(=O)(=O)Cc1ccc([N+](=O)[O-])cc1]

The title compound was prepared according to General Procedure 3 starting from Compound 1.2 (20 mg) and (4-nitrophenyl)methanesulfonyl chloride. Preparative HPLC purification was accomplished as described in General Procedure 9, eluting with a 10 to 50% CH₃CN/H₂O+0.1% TFA gradient to give the title compound as an off-white solid (5.0 mg, 17% yield).

LC/MS: Calc'd m/z=608.1 for C₂₉H₂₅FN₄O₈S, found [M+H]⁺=609.2.

¹H NMR (300 MHz, CDCl₃) δ 8.02-7.92 (m, 3H), 7.74 (d, J=10.5 Hz, 1H), 7.65 (s, 1H), 7.33 (d, J=8.6 Hz, 2H), 5.66 (d, J=16.8 Hz, 1H), 5.28 (d, J=16.5 Hz, 1H), 5.14 (d, J=5.4 Hz, 2H), 4.67 (s, 2H), 4.28 (d, J=6.3 Hz, 2H), 3.39 (s, 3H), 2.03-1.83 (m, 2H), 1.04 (t, J=7.4 Hz, 3H).

### 1.22: (S)—N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)benzenesulfonamide (Compound 125)

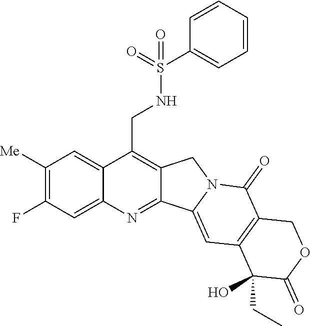 [SMILES: CC[C@@]1(O)C(=O)OCc2c1cc1n(c2=O)Cc2c-1nc1cc(F)c(C)cc1c2CNS(=O)(=O)c1ccccc1]

The title compound was prepared according to General Procedure 3 starting from Compound 1.2 (10 mg) and benzenesulfonyl chloride. Preparative HPLC purification was accomplished as described in General Procedure 9, eluting with a 10 to 50% CH₃CN/H₂O+0.1% TFA gradient to give the title compound as an off-white solid (9.8 mg, 73% yield).

LC/MS: Calc'd m/z=549.6 for C₂₈H₂₄FN₃O₆S, found [M+H]⁺=550.6.

¹H NMR (300 MHz, DMSO-d6) δ 8.60 (t, J=6.2 Hz, 1H), 8.17 (d, J=8.1 Hz, 1H), 7.83 (d, J=10.8 Hz, 1H), 7.71 (dd, J=7.1, 1.7 Hz, 2H), 7.66-7.48 (m, 2H), 7.46 (dd, J=8.3, 6.8 Hz, 2H), 7.40-7.27 (m, 2H), 7.18 (s, 1H), 7.01 (s, 1H), 5.45 (s, 2H), 5.33 (s, 2H), 4.63 (d, J=6.2 Hz, 2H), 2.48 (s, 3H), 1.98-1.76 (m, 2H), 0.89 (t, J=7.3 Hz, 3H).

### 1.23: (S)—N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-4-nitrobenzenesulfonamide (Compound 1.23)

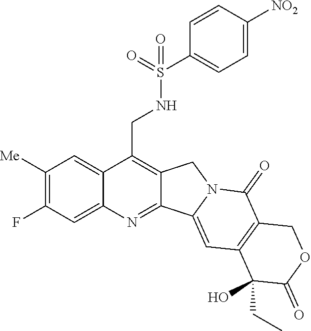 [SMILES: CC[C@@]1(O)C(=O)OCc2c1cc1n(c2=O)Cc2c-1nc1cc(F)c(C)cc1c2CNS(=O)(=O)c1ccc([N+](=O)[O-])cc1]

The title compound was prepared according to General Procedure 3 starting from Compound 1.2 (75 mg) and 4-nitrobenzenesulfonyl chloride. Purification of the title compound was accomplished as described in General Procedure 9, using a 12 g C18 column and eluting with a 5 to 75% CH₃CN/H₂O+0.1% TFA gradient to give the title compound as an off-white solid (37.8 mg, 47% yield).

LC/MS: Calc'd m/z=594.6 for C₂₈H₂₃FN₄O₈S, found [M+H]⁺=595.2.

### 1.24: (S)-4-amino-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)benzenesulfonamide (Compound 127)

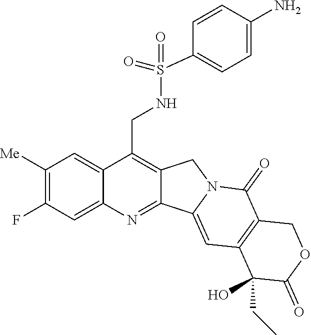 [SMILES: CC[C@@]1(O)C(=O)OCc2c1cc1n(c2=O)Cc2c-1nc1cc(F)c(C)cc1c2CNS(=O)(=O)c1ccc(N)cc1]

To a solution of Compound 1.23 (37.8 mg, 0.064 mmol) in methanol (6.4 mL) was added platinum 1% vanadium 2% on carbon (75 mg). The flask was purged with H₂ then stirred at room temperature under an H₂ atmosphere for 45 min. The mixture was filtered through a pad of celite, washed with DMF, and the filtrate evaporated to give the title compound as a pale yellow solid (30 mg, 84% yield).

LC/MS: Calc'd m/z=564.6 for C₂₈H₂₄FN₄O₆S, found [M+H]⁺=565.2.

¹H NMR (300 MHz, DMSO-d6) δ 8.13 (d, J=8.2 Hz, 1H), 8.02 (t, J=6.2 Hz, 1H), 7.88 (d, J=10.8 Hz, 1H), 7.48-7.35 (m, 2H), 7.31 (d, J=8.4 Hz, 1H), 6.63-6.45 (m, 2H), 5.45 (s, 2H), 5.36 (s, 2H), 4.50 (d, J=6.3 Hz, 2H), 1.98-1.75 (m, 2H), 0.89 (t, J=7.3 Hz, 3H).

### 1.25: (S)—N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-2-hydroxyethane-1-sulfonamide (Compound 129)

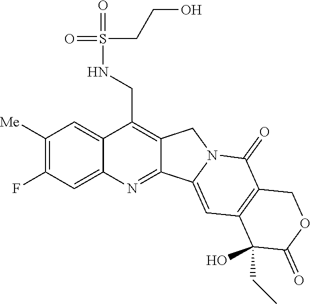 [SMILES: CC[C@@]1(O)C(=O)OCc2c1cc1n(c2=O)Cc2c-1nc1cc(F)c(C)cc1c2CNS(=O)(=O)CCO]

The title compound was prepared according to General Procedure 3 starting from Compound 1.2 (20 mg) and 2-hydroxyethanesulfonyl chloride. Preparative HPLC purification was accomplished as described in General Procedure 9, eluting with a 25 to 50% CH₃CN/H₂O+0.1% TFA gradient to give the title compound as an off-white solid (1.3 mg, 13% yield).

LC/MS: Calc'd m/z=517.1 for C₂₄H₂₄FN₃O₇S, found [M+H]⁺=518.2.

¹H NMR (300 MHz, DMSO-d6) δ 8.30 (d, J=8.4 Hz, 1H), 7.91 (d, J=10.9 Hz, 1H), 7.84 (t, J=6.3 Hz, 1H), 7.33 (s, 1H), 5.50-5.33 (m, 4H), 5.07 (t, J=5.4 Hz, 1H), 4.78 (d, J=6.0 Hz, 2H), 4.07 (s, 3H), 3.80 (dt, J=6.3 Hz, J=5.8 Hz, 2H), 1.86 (m, 2H), 0.87 (d, J=7.3 Hz, 3H).

### 1.26: (S)—N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)methanesulfamide (Compound 131)

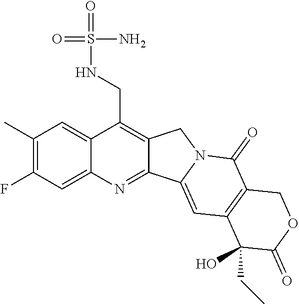 [SMILES: CC[C@@]1(O)C(=O)OCc2c1cc1n(c2=O)Cc2c-1nc1cc(F)c(C)cc1c2CNS(N)(=O)=O]

To a solution of chlorosulfonyl isocyanate (3 μL) in dichloromethane (1 mL) was added tert-butanol (3 μL). This solution was stirred for 1 h, then Compound 1.2 (13 mg) dissolved in dichloromethane (1 mL) was added followed by triethylamine (13 μL). The reaction was stirred for 1 hr then concentrated to dryness. Preparative HPLC purification of the intermediate Boc compound was accomplished as described in General Procedure 9, eluting with a 10 to 50% CH₃CN/H₂O+0.1% TFA gradient. To the purified solid in dichloromethane (1 mL) was added trifluoroacetic acid (200 μL). The reaction was stirred for 16 h then concentrated to dryness to provide the title compound as an off-white solid (7.5 mg, 48% yield).

LC/MS: Calc'd m/z=488.1 for C₂₂H₂₁FN₄O₆S, found [M+H]⁺=489.0.

¹H NMR (300 MHz, MeOD) δ 8.25 (d, J=8.1 Hz, 1H), 7.73 (d, J=10.7 Hz, 1H), 7.62 (s, 1H), 5.59 (d, J=16.4 Hz, 1H), 5.45 (s, 2H), 5.39 (d, J=16.4 Hz, 1H), 4.81 (s, 2H), 2.55 (d, J=1.7 Hz, 3H), 2.07-1.89 (m, 2H), 1.03 (t, J=7.4 Hz, 3H).

### 1.27: 4-nitrophenyl-(S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound

### 1.27)

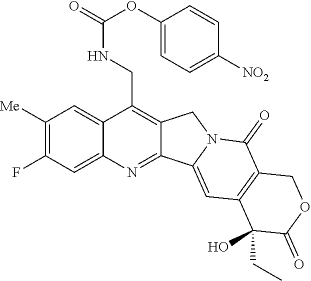 [SMILES: CC[C@@]1(O)C(=O)OCc2c1cc1n(c2=O)Cc2c-1nc1cc(F)c(C)cc1c2CNC(=O)Oc1ccc([N+](=O)[O-])cc1]

The title PNP-carbamate intermediate compound was prepared according to the first step of General Procedure 4 starting from Compound 1.2 (24 mg). Purification was accomplished as described in General Procedure 9, using a 12 g column C18 column and eluting with a 10 to 50% CH₃CN/H₂O+0.1% TFA gradient to give the title compound as an off-white solid (14 mg, 53% yield).

LC/MS: Calc'd m/z=574.2 for C₂₉H₂₃FN₄O₈S, found [M+H]⁺=575.2

### 1.28: (S)-1-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-methylurea (Compound 132)

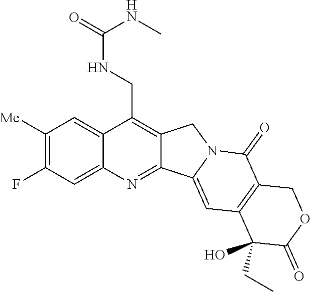 [SMILES: CC[C@@]1(O)C(=O)OCc2c1cc1n(c2=O)Cc2c-1nc1cc(F)c(C)cc1c2CNC(=O)NC]

The title compound was prepared according to General Procedure 4 starting from Compound 1.2 (25 mg) and aqueous methyl amine (500 μL, 40 wt. % in water) as the primary amine. In this instance, the intermediate PNP-carbamate was used crude. Preparative HPLC purification was accomplished as described in General Procedure 9, eluting with a 10 to 50% CH₃CN/H₂O+0.1% TFA gradient to give the title compound as an off-white solid (8.9 mg, 31% yield).

LC/MS: Calc'd m/z=466.2 for C₂₄H₂₃FN₄O₅, found [M+H]⁺=467.2.

¹H NMR (300 MHz, MeOD) δ 8.26 (d, J=8.2 Hz, 1H), 7.79 (d, J=10.7 Hz, 1H), 7.66 (s, 1H), 5.61 (d, J=16.3 Hz, 1H), 5.48 (s, 2H), 5.41 (d, J=16.4 Hz, 1H), 4.97 (s, 2H), 2.73 (s, 3H), 2.57 (s, 3H), 2.08-1.93 (m, 2H), 1.03 (t, J=7.4 Hz, 3H).

### 1.29: (S)-1-(4-aminobenzyl)-3-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)urea (Compound 134)

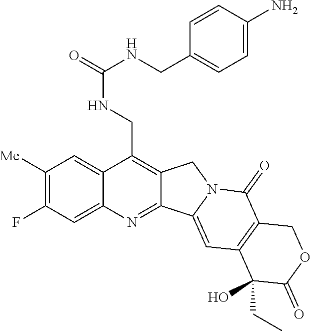 [SMILES: CC[C@@]1(O)C(=O)OCc2c1cc1n(c2=O)Cc2c-1nc1cc(F)c(C)cc1c2CNC(=O)NCc1ccc(N)cc1]

The title compound was prepared according to the second step of General Procedure 4 using Compound 1.27 (4 mg) as the PNP-carbamate and 4-(aminomethyl)aniline as the primary amine. Preparative HPLC purification was accomplished as described in General Procedure 9, eluting with a 20 to 50% CH₃CN/H₂O+0.1% TFA gradient to give the title compound as an off-white solid (0.6 mg, 12% yield).

LC/MS: Calc'd m/z=557.2 for C₃₀H₂₈FN₅O₅, found [M+H]⁺=558.4.

¹H NMR (300 MHz, MeOD) δ 8.25 (d, J=8.1 Hz, 1H), 7.80 (d, J=10.8 Hz, 1H), 7.67 (s, 1H), 7.43 (d, J=8.2 Hz, 2H), 7.24 (d, J=8.3 Hz, 2H), 5.63 (d, J=16.4 Hz, 1H), 5.48 (s, 2H), 5.43 (d, J=16.4 Hz, 1H), 5.01 (s, 2H), 4.37 (s, 2H), 2.56 (d, J=1.7 Hz, 3H), 2.05-1.94 (m, 2H), 1.03 (t, J=7.3 Hz, 3H).

### 1.30: (S)-1-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-(2-hydroxyethyl)urea (Compound 136)

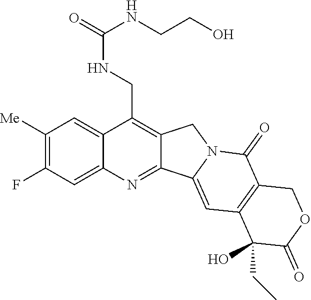 [SMILES: CC[C@@]1(O)C(=O)OCc2c1cc1n(c2=O)Cc2c-1nc1cc(F)c(C)cc1c2CNC(=O)NCCO]

The title compound was prepared according to the second step of General Procedure 4 using Compound 1.27 (4 mg) as the PNP-carbamate and hydroxyethylamine as the primary amine. Preparative HPLC purification was accomplished as described in General Procedure 9, eluting with a 10 to 50% CH₃CN/H₂O+0.1% TFA gradient to give the title compound as an off-white solid (2.4 mg, 66% yield).

LC/MS: Calc'd m/z=496.2 for C₂₅H₂₅FN₄O₆, found [M+H]⁺=497.2.

¹H NMR (300 MHz, MeOD) δ 8.08 (d, J=8.0 Hz, 1H), 7.74 (d, J=10.5 Hz, 1H), 7.68 (s, 1H), 5.64 (d, J=16.4 Hz, 1H), 5.41 (s, 2H), 5.31 (d, J=16.4 Hz, 1H), 4.96 (s, 2H), 3.63 (t, J=5.2 Hz, 2H), 3.29 (t, J=5.3 Hz, 2H), 2.54 (s, 3H), 1.98-1.87 (m, 2H), 1.01 (t, J=7.4 Hz, 3H).

### 1.31: Methyl-(S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 138)

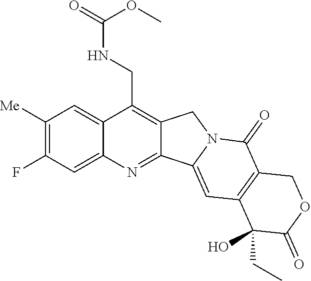 [SMILES: CC[C@@]1(O)C(=O)OCc2c1cc1n(c2=O)Cc2c-1nc1cc(F)c(C)cc1c2CNC(=O)OC]

The title compound was prepared according to General Procedure 5 starting from Compound 1.2 (50 mg) and reacting methanol with the intermediate PNP-carbamate. Preparative HPLC purification was accomplished as described in General Procedure 9, eluting with a 20 to 50% CH₃CN/H₂O+0.1% TFA gradient to give the title compound as an off-white solid (3.5 mg, 6% yield).

LC/MS: Calc'd m/z=467.2 for C₂₄H₂₂FN₃O₆, found [M+H]⁺=468.2.

¹H NMR (300 MHz, MeOD) δ 8.17 (d, J=8.2 Hz, 1H), 7.77 (d, J=10.5 Hz, 1H), 7.69 (s, 1H), 5.65 (d, J=16.5 Hz, 1H), 5.48 (s, 2H), 5.33 (d, J=16.4 Hz, 1H), 4.86 (d, J=5.6 Hz, 2H), 3.65 (s, 3H), 2.56 (s, 3H), 2.02-1.89 (m, 2H), 1.02 (t, J=7.4 Hz, 3H).

### 1.32: 2-hydroxyethyl (S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 139)

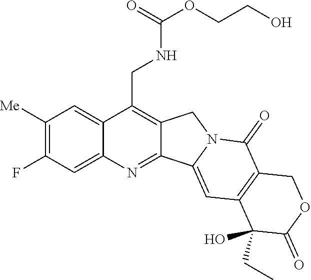 [SMILES: CC[C@@]1(O)C(=O)OCc2c1cc1n(c2=O)Cc2c-1nc1cc(F)c(C)cc1c2CNC(=O)OCCO]

The title compound was prepared according to General Procedure 5 starting from Compound 1.2 (18 mg) and reacting 1,2-ethanediol with the intermediate PNP-carbamate. Preparative HPLC purification was accomplished as described in General Procedure 9, eluting with a 10 to 60% CH₃CN/H₂O+0.1% TFA gradient to give the title compound as an off-white solid (4.2 mg, 19% yield).

LC/MS: Calc'd m/z=497.2 for C₂₅H₂₄FN₃O₇, found [M+H]⁺=498.2.

¹H NMR (300 MHz, DMSO) δ 8.23 (d, J=8.2 Hz, 1H), 7.78 (d, J=10.7 Hz, 1H), 7.40 (s, 1H), 5.47 (d, J=16.5 Hz, 1H), 5.42 (s, 2H), 5.34 (d, J=16.4 Hz, 1H), 4.77 (s, 2H), 3.99 (t, J=4.9 Hz, 2H), 3.64-3.38 (m, 2H), 2.48 (s, 3H), 2.02-1.67 (m, 2H), 0.89 (t, J=7.3 Hz, 3H).

### Example 2: Preparation of Camptothecin Analogues Having Methoxy at the C10 Position

### 2.1: 1-(2-amino-4-fluoro-5-methoxyphenyl)-2-chloroethan-1-one (Compound 2.1)

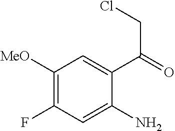 [SMILES: COc1cc(C(=O)CCl)c(N)cc1F]

A solution of 3-fluoro-4-methoxyaniline (10 g, 71 mmol) in DCM (100 mL) was cooled to 0° C. To this solution was first added a 1 M BCl₃ in DCM (71 mL, 71 mmol), followed by a 1 M chloro(diethyl)alumane in DCM (71 mL, 71 mmol), then finally 2-chloroacetonitrile (6.4 g, 85 mmol). The solution was heated at reflux for 3 h, cooled to room temperature, and quenched by the addition of an aqueous 2 M HCl solution. The resulting heterogenous mixture was heated to reflux for 1 h, cooled to room temperature, then the pH was adjusted to ~12 with Na₂CO₃. The layers were separated, and the aqueous layer extracted with DCM (3×100 mL). The combined organic layers were dried over Na₂SO₄, concentrated, and flash purified as described in General Procedure 9, eluting with 0 to 20% EtOAc/Hexanes to give the title compound (6 g, 28 mmol, 39% yield).

LC/MS: Calc'd m/z=217.1 for C₉H₉ClFNO₂, found [M+H]⁺=218.1.

¹H NMR (400 MHz, CDCl₃) δ 7.19 (d, J=9.2 Hz, 1H), 6.44 (d, J=12.8 Hz, 1H), 4.59 (s, 2H), 3.86 (s, 3H)

### 2.2: (S)-11-(chloromethyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 2.2)

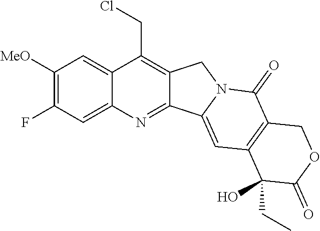 [SMILES: CC[C@@]1(O)C(=O)OCc2c1cc1n(c2=O)Cc2c-1nc1cc(F)c(OC)cc1c2CCl]

To a solution of Compound 2.1 (1.65 g, 7.6 mmol) and (S)-4-ethyl-4-hydroxy-7,8-dihydro-1H-pyrano[3,4-f]indolizine-3,6,10(4H)-trione (2 g, 7.6 mmol) in toluene (200 mL) was added toluene-4-sulfonic acid (157 mg, 0.9 mmol). This solution was heated at 140° C. for 3 h then cooled to room temperature. The product as yellow precipitate was collected by filtration to give the title compound (1.27 g, 2.85 mmol, 37.5% yield).

LC/MS: Calc'd m/z=445.2 for C₂₂H₁₈C₁FN₂O₅, found [M+H]⁺=445.1.

¹H NMR (400 MHz, DMSO-d6) δ 7.99 (d, J=12.0 Hz, 1H) 7.80 (d, J=9.2 Hz, 1H) 7.27 (s, 1H), 6.50 (s, 1H), 5.45 (s, 2H), 5.41 (s, 2H), 5.33 (s, 2H) 4.08 (s, 3H), 1.87-1.83 (m, 2H), 0.87 (t, J=7.2 Hz, 3H)

### 2.3: (S)-4-ethyl-8-fluoro-4-hydroxy-9-methoxy-11-(morpholinomethyl)-1,12-dihydro-14H-pyrano[3′,4′:6,7]indolizino[1,2-b]quinoline-3,14(4H)-dione (Compound 101)

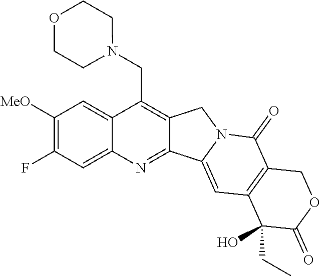 [SMILES: CC[C@@]1(O)C(=O)OCc2c1cc1n(c2=O)Cc2c-1nc1cc(F)c(OC)cc1c2CN1CCOCC1]

The title compound was prepared according to General Procedure 1 starting from Compound 2.2 (10 mg) and morpholine. Preparative HPLC purification was accomplished as described in General Procedure 9, eluting with a 20 to 60% CH₃CN/H₂O+0.1% TFA gradient to give the title compound as an off-white solid (5.6 mg, 41% yield).

LC/MS: Calc'd m/z=495.2 for C₂₆H₂₆FN₃O₆, found [M+H]⁺=496.4.

¹H NMR (300 MHz, MeOD) δ 7.84-7.70 (m, 2H), 7.59 (s, 1H), 5.62 (d, J=16.3 Hz, 1H), 5.45-5.36 (m, 3H), 4.29 (s, 2H), 4.12 (s, 3H), 3.58-3.48 (m, 2H), 3.28-3.09 (m, 2H), 2.75-2.61 (m, 2H), 2.05-1.91 (m, 2H), 1.02 (t, J=7.4 Hz, 3H).

### 2.4: (S)-4-ethyl-8-fluoro-4-hydroxy-9-methoxy-11-((4-(phenylsulfonyl)piperazin-1-yl)methyl)-1,12-dihydro-14H-pyrano[3′,4′:6,7]indolizino[1,2-b]quinoline-3,14(4H)-dione (Compound 103)

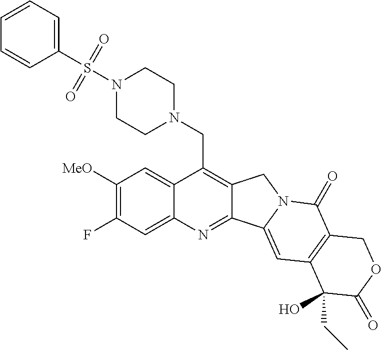 [SMILES: CC[C@@]1(O)C(=O)OCc2c1cc1n(c2=O)Cc2c-1nc1cc(F)c(OC)cc1c2CN1CCN(S(=O)(=O)c2ccccc2)CC1]

The title compound was prepared according to General Procedure 1 starting from Compound 2.2 (10 mg) and 1-(phenylsulfonyl)piperazine. Preparative HPLC purification was accomplished as described in General Procedure 9, eluting with a 20 to 60% CH₃CN/H₂O+0.1% TFA gradient to give the title compound as an off-white solid (2.5 mg, 14% yield).

LC/MS: Calc'd m/z=634.2 for C₃₂H₃₁FN₄O₇S, found [M+H]⁺=635.4.

### 2.5: (S)-11-((4-((4-aminophenyl)sulfonyl)piperazin-1-yl)methyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 105)

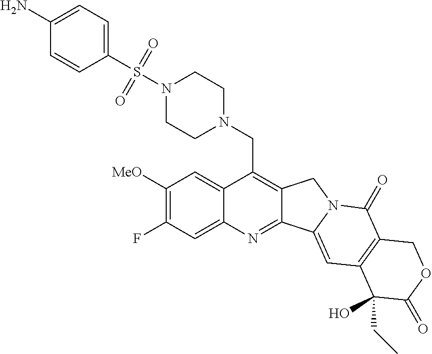 [SMILES: CC[C@@]1(O)C(=O)OCc2c1cc1n(c2=O)Cc2c-1nc1cc(F)c(OC)cc1c2CN1CCN(S(=O)(=O)c2ccc(N)cc2)CC1]

The title compound was prepared according to General Procedure 1 starting from Compound 2.2 (10 mg) and 4-(piperazin-1-ylsulfonyl)aniline. Preparative HPLC purification was accomplished as described in General Procedure 9, eluting with a 20 to 60% CH₃CN/H₂O+0.1% TFA gradient to give the title compound as an off-white solid (4.0 mg, 23% yield).

LC/MS: Calc'd m/z=649.2 for C₃₂H₃₂FN₅O₇S, found [M+H]⁺=650.4.

¹H NMR (300 MHz, DMSO) δ 8.08 (s, 2H), 7.90-7.67 (m, 2H), 7.35 (s, 1H), 7.32-7.26 (m, 2H), 6.67-6.57 (m, 2H), 5.46 (d, J=16.5 Hz, 1H), 5.33-5.22 (m, 3H), 3.92 (s, 3H), 3.02-2.72 (m, 4H), 2.75-2.58 (m, 4H), 1.97-1.70 (m, 2H), 0.90 (t, J=7.3 Hz, 3H).

### 2.6: (S)-4-ethyl-8-fluoro-4-hydroxy-9-methoxy-11-((4-methylpiperazin-1-yl)methyl)-1,12-dihydro-14H-pyrano[3′,4′:6,7]indolizino[1,2-b]quinoline-3,14(4H)-dione (Compound 107)

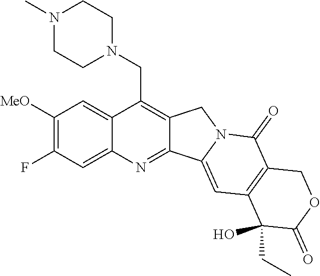 [SMILES: CC[C@@]1(O)C(=O)OCc2c1cc1n(c2=O)Cc2c-1nc1cc(F)c(OC)cc1c2CN1CCN(C)CC1]

The title compound was prepared according to General Procedure 1 starting from Compound 2.2 (10 mg) and N-methylpiperazine. Preparative HPLC purification was accomplished as described in General Procedure 9, eluting with a 20 to 60% CH₃CN/H₂O+0.1% TFA gradient to give the title compound as an off-white solid (2.1 mg, 19% yield).

LC/MS: Calc'd m/z=508.2 for C₂₇H₂₉FN₄O₅, found [M+H]⁺=509.4.

### 2.7: (S)-11-((4-(4-aminophenyl)piperazin-1-yl)methyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 109)

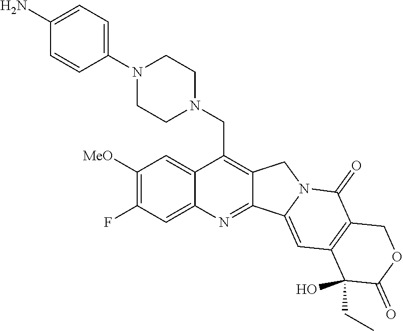 [SMILES: CC[C@@]1(O)C(=O)OCc2c1cc1n(c2=O)Cc2c-1nc1cc(F)c(OC)cc1c2CN1CCN(c2ccc(N)cc2)CC1]

The title compound was prepared according to General Procedure 1 starting from Compound 2.2 (10 mg) and 4-(piperazin-1-yl)aniline. Preparative HPLC purification was accomplished as described in General Procedure 9, eluting with a 20 to 60% CH₃CN/H₂O+0.1% TFA gradient to give the title compound as an off-white solid (3.2 mg, 20% yield).

LC/MS: Calc'd m/z=585.2 for C₃₂H₃₂FN₅O₅, found [M+H]⁺=586.4.

¹H NMR (300 MHz, MeOD) δ 7.83-7.74 (m, 2H), 7.62 (s, 1H), 7.06 (d, J=8.9 Hz, 2H), 6.98 (d, J=8.9 Hz, 2H), 5.65 (d, J=16.4 Hz, 1H), 5.36 (s, 2H), 5.27 (d, J=16.4 Hz, 1H), 4.13 (s, 2H), 4.06 (s, 3H), 3.26 (br s, 4H), 2.79 (br s, 4H), 1.97-1.83 (m, 2H), 1.00 (t, J=7.4 Hz, 3H).

### 2.8: (S)-11-(aminomethyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 2.8)

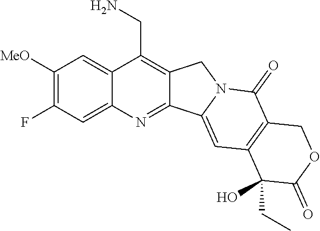 [SMILES: CC[C@@]1(O)C(=O)OCc2c1cc1n(c2=O)Cc2c-1nc1cc(F)c(OC)cc1c2CN]

To a solution of Compound 2.2 (250 mg, 0.56 mmol) in ethanol (7 mL) was added hexamethylenetetramine (236 mg, 1.7 mmol) followed by iPr₂NEt (100 μL, 0.56 mmol). This solution was heated at reflux for 5 h, cooled to room temperature and quenched with 12 M aqueous HCl (60 μL). This solution was concentrated to ~½ volume and 1 M aqueous HCl (1.5 mL) was added, stirred for 5 min, then concentrated to give a brown residue. Purification was accomplished as described in General Procedure 9, using a 12 g C18 flash column and eluting with a 5 to 40% CH₃CN/H₂O+0.1% TFA gradient to give the title compound as pale yellow solid (179 mg, 75% yield).

LC/MS: Calc'd m/z=425.4 for C₂₂H₂₀FN₃O₅, found [M+H]⁺=426.2

### 2.9: (S)—N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)methanesulfonamide (Compound 123)

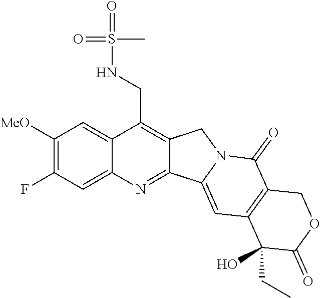 [SMILES: CC[C@@]1(O)C(=O)OCc2c1cc1n(c2=O)Cc2c-1nc1cc(F)c(OC)cc1c2CNS(C)(=O)=O]

The title compound was prepared according to General Procedure 3 starting from Compound 2.8 (10 mg) and methanesulfonyl chloride. Preparative HPLC purification was accomplished as described in General Procedure 9, eluting with a 5 to 65% CH₃CN/H₂O+0.1% TFA gradient to give the title compound as an off-white solid (8.5 mg, 91% yield).

LC/MS: Calc'd m/z=503.1 for C₂₃H₂₂FN₃O₇S, found [M+H]⁺=504.2.

¹H NMR (300 MHz, DMSO-d6) δ 7.98 (d, J=12.1 Hz, 1H), 7.89 (t, J=6.4 Hz, 1H), 7.80 (d, J=9.1 Hz, 1H), 7.28 (s, 1H), 5.42 (s, 2H), 5.39 (s, 2H), 4.77 (d, J=6.4 Hz, 2H), 4.06 (s, 3H), 3.06 (s, 3H), 1.95-1.73 (m, 2H), 0.88 (d, J=7.3 Hz, 3H).

### 2.10: (S)—N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)benzenesulfonamide (Compound 126)

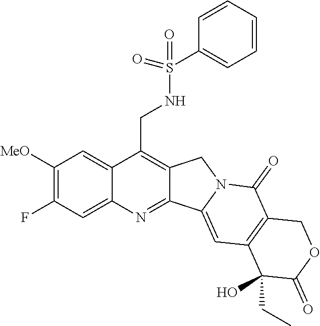 [SMILES: CC[C@@]1(O)C(=O)OCc2c1cc1n(c2=O)Cc2c-1nc1cc(F)c(OC)cc1c2CNS(=O)(=O)c1ccccc1]

The title compound was prepared according to General Procedure 3 starting from Compound 2.8 (7.5 mg) and benzenesulfonyl chloride. Preparative HPLC purification was accomplished as described in General Procedure 9, eluting with a 5 to 70% CH₃CN/H₂O+0.1% TFA gradient to give the title compound as an off-white solid (4.6 mg, 46% yield).

LC/MS: Calc'd m/z=565.6 for C₂₈H₂₄FN₃O₇S, found [M+H]⁺=566.2.

¹H NMR (300 MHz, DMSO-d6) δ 8.59 (t, J=6.3 Hz, 1H), 7.94 (d, J=12.2 Hz, 1H), 7.82-7.68 (m, 2H), 7.62-7.46 (m, 1H), 7.51-7.40 (m, 1H), 7.28 (d, J=8.3 Hz, 1H), 6.52 (s, 1H), 5.44 (s, 1H), 5.36 (s, 1H), 4.64 (d, J=6.3 Hz, 1H), 4.09 (s, 2H), 1.95-1.81 (m, 1H), 0.89 (t, J=7.3 Hz, 2H).

### 2.11: (S)—N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-4-nitrobenzenesulfonamide (Compound 2.11)

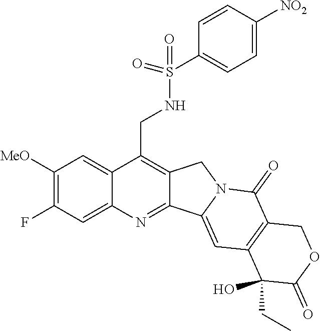 [SMILES: CC[C@@]1(O)C(=O)OCc2c1cc1n(c2=O)Cc2c-1nc1cc(F)c(OC)cc1c2CNS(=O)(=O)c1ccc([N+](=O)[O-])cc1]

The title compound was prepared according to General Procedure 3 starting from Compound 2.8 (12 mg) and 4-nitrobenzenesulfonyl chloride. Purification was accomplished as described in General Procedure 9 using a 12 g C18 flash column and eluting with a 5 to 75% CH₃CN/H₂O+0.1% TFA gradient to give the title compound as pale yellow solid (9.7 mg, 71% yield).

LC/MS: Calc'd m/z=610.6 for C₂₈H₂₃FN₄O₉S, found [M+H]⁺=611.5.

### 2.12: (S)-4-amino-N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)benzenesulfonamide (Compound 128)

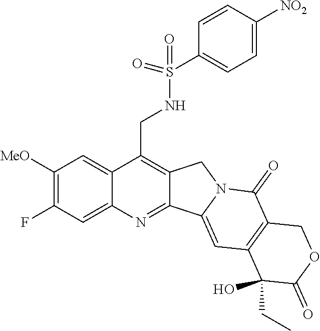 [SMILES: CC[C@@]1(O)C(=O)OCc2c1cc1n(c2=O)Cc2c-1nc1cc(F)c(OC)cc1c2CNS(=O)(=O)c1ccc([N+](=O)[O-])cc1]

To a solution of Compound 2.11 (9.7 mg, 0.016 mmol) in methanol (1.6 mL) was added platinum 1% vanadium 2% on carbon (15 mg). The flask was purged with H₂ then stirred at room temperature under an H₂ atmosphere for 45 min. The mixture was filtered through a pad of celite, washed with DMF, then the filtrate was evaporated to give the title compound as a pale yellow solid (1.5 mg, 16% yield).

LC/MS: Calc'd m/z=580.6 for C₂₈H₂₅FN₄O₇S, found [M+H]⁺=581.4.

¹H NMR (300 MHz, MeOD) δ 7.77 (d, J=11.0 Hz, 1H), 7.58 (s, 1H), 7.48 (d, J=8.6 Hz, 1H), 6.61 (d, J=8.6 Hz, 1H), 5.59 (d, J=16.3 Hz, 1H), 5.39 (d, J=16.4 Hz, 1H), 5.30 (s, 1H), 4.56 (s, 1H), 4.10 (d, J=3.7 Hz, 3H), 2.04-1.91 (m, 2H), 1.31 (s, 1H), 1.02 (t, J=7.3 Hz, 3H), 0.90 (s, 1H).

### 2.13: (S)—N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-2-hydroxyethane-1-sulfonamide (Compound 130)

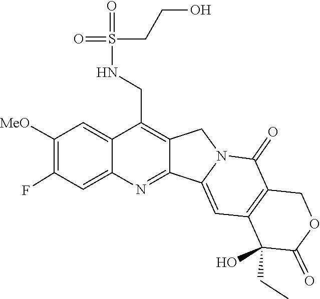 [SMILES: CC[C@@]1(O)C(=O)OCc2c1cc1n(c2=O)Cc2c-1nc1cc(F)c(OC)cc1c2CNS(=O)(=O)CCO]

The title compound was prepared according to General Procedure 3 starting from Compound 2.8 (8 mg) and 2-hydroxyethanesulfonyl chloride. Preparative HPLC purification was accomplished as described in General Procedure 9, eluting with a 15 to 50% CH₃CN/H₂O+0.1% TFA gradient to give the title compound as an off-white solid (2.2 mg, 22% yield).

LC/MS: Calc'd m/z=533.1 for C₂₄H₂₄FN₃O₈S found [M+H]⁺=534.2.

¹H NMR (300 MHz, DMSO-d6) δ 7.99 (d, J=12.2 Hz, 1H), 7.89-7.79 (m, 2H), 7.29 (s, 1H), 5.43 (s, 2H), 5.40 (s, 2H), 4.76 (d, J=6.4 Hz, 2H), 4.06 (s, 3H), 3.81 (t, J=6.3 Hz, 2H), 3.34 (t, J=6.3 Hz, 2H), 1.94-1.75 (m, 2H), 0.87 (d, J=7.4 Hz, 3H).

### 2.14: 4-nitrophenyl-(S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 2.14)

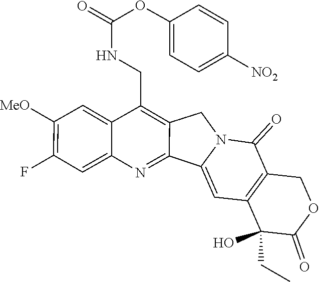 [SMILES: CC[C@@]1(O)C(=O)OCc2c1cc1n(c2=O)Cc2c-1nc1cc(F)c(OC)cc1c2CNC(=O)Oc1ccc([N+](=O)[O-])cc1]

The title PNP-carbamate intermediate compound was prepared according to the first step of General Procedure 4 starting from Compound 2.8 (65 mg) and using a 1:1 mixture of dimethylformamide and dichloromethane as the solvent. Flash purification was accomplished as described in General Procedure 9, using a 12 g C₁₂ column and eluting with a 10 to 50% CH₃CN/H₂O+0.1% TFA gradient to give the title compound as an off-white solid (61 mg, 86% yield). This intermediate was divided and used to generate the following compounds.

LC/MS: Calc'd m/z=590.1 for C₂₉H₂₃FN₄O₉, found [M+H]⁺=591.2.

### 2.15: (S)-1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-3-methylurea (Compound 133)

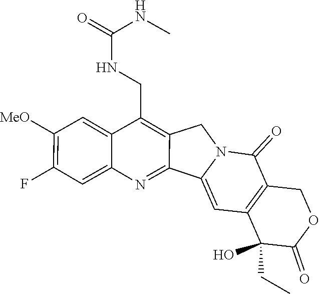 [SMILES: CC[C@@]1(O)C(=O)OCc2c1cc1n(c2=O)Cc2c-1nc1cc(F)c(OC)cc1c2CNC(=O)NC]

The title compound was prepared according to second step of General Procedure 4 using Compound 2.14 (15 mg) as the PNP-carbamate and aqueous methyl amine (500 uL, 40 wt. % in water) as the primary amine. Preparative HPLC purification was accomplished as described in General Procedure 9, eluting with a 20 to 60% CH₃CN/H₂O+0.1% TFA gradient to give the title compound as an off-white solid (5.8 mg, 47% yield).

LC/MS: Calc'd m/z=482.2 for C₂₄H₂₃FN₄O₆, found [M+H]⁺=483.2.

¹H NMR (300 MHz, DMSO-d6) δ 8.00-7.87 (m, 2H), 7.31 (s, 1H), 5.48-5.39 (m, 3H), 4.81 (s, 3H), 2.56 (s, 3H), 1.93-1.81 (m, 2H), 0.89 (t, J=7.3 Hz, 3H).

### 2.16: (S)-1-(4-aminobenzyl)-3-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)urea (Compound 135)

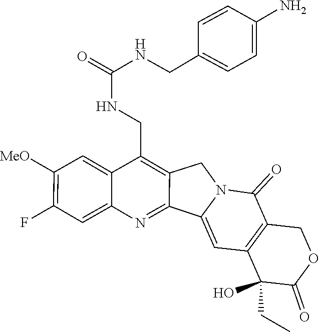 [SMILES: CC[C@@]1(O)C(=O)OCc2c1cc1n(c2=O)Cc2c-1nc1cc(F)c(OC)cc1c2CNC(=O)NCc1ccc(N)cc1]

The title compound was prepared according to the second step of General Procedure 4 using Compound 2.14 (15 mg) as the PNP-carbamate and 4-(aminomethyl)aniline as the primary amine. Preparative HPLC purification was accomplished as described in General Procedure 9, eluting with a 20 to 60% CH₃CN/H₂O+0.1% TFA gradient to give the title compound as an off-white solid (TFA salt, 2.1 mg, 12% yield).

LC/MS: Calc'd m/z=573.2 for C₃₀H₂₈FN₅O₆, found [M+H]⁺=574.2.

¹H NMR (300 MHz, MeOD) δ 7.79 (d, J=11.9 Hz, 1H), 7.74 (d, J=9.0 Hz, 1H), 7.59 (s, 1H), 7.43 (d, J=8.2 Hz, 2H), 7.25 (d, J=8.2 Hz, 2H), 5.61 (d, J=16.3 Hz, 1H), 5.52-5.35 (m, 3H), 4.98 (s, 2H), 4.39 (s, 2H), 4.01 (s, 3H), 2.03-1.93 (m, 2H), 1.03 (t, J=7.4 Hz, 3H).

### 2.17: (S)-1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-3-(2-hydroxyethyl)urea (Compound 137)

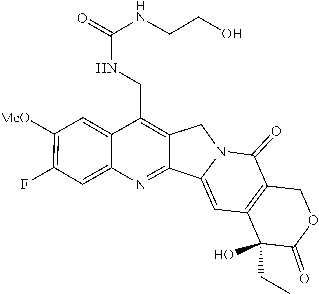 [SMILES: CC[C@@]1(O)C(=O)OCc2c1cc1n(c2=O)Cc2c-1nc1cc(F)c(OC)cc1c2CNC(=O)NCCO]

The title compound was prepared according to the second step of General Procedure 4 using Compound 2.14 (15 mg) as the PNP-carbamate and hydroxyethylamine as the primary amine. Preparative HPLC purification was accomplished as described in General Procedure 9, eluting with a 20 to 60% CH₃CN/H₂O+0.1% TFA gradient to give the title compound as an off-white solid (1.5 mg, 12% yield).

LC/MS: Calc'd m/z=512.2 for C₂₅H₂₅FN₄O₇, found [M+H]⁺=513.2.

¹H NMR (300 MHz, MeOD) δ 7.93 (d, J=12.1 Hz, 1H), 7.88 (d, J=9.2 Hz, 1H), 7.56 (s, 1H), 5.62 (d, J=16.2 Hz, 1H), 5.52 (s, 2H), 5.45 (d, J=16.3 Hz, 1H), 4.98 (s, 2H), 4.17 (s, 3H), 3.59 (t, J=5.6 Hz, 2H), 3.28 (t, J=5.6 Hz, 2H), 2.10-1.91 (m, 2H), 1.05 (t, J=7.3 Hz, 3H).

### Example 3: Preparation of Camptothecin Analogues Having Amino at the C10 Position

### 3.1: 5-bromo-4-fluoro-2-nitrobenzaldehyde (Compound 3.1)

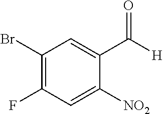 [SMILES: O=Cc1cc(Br)c(F)cc1[N+](=O)[O-]]

To a stirring solution of HNO₃ (121.2 mL, 67% purity, 2.0 eq.) in H₂SO₄ (500 mL) at 0° C. was added 3-bromo-4-fluorobenzaldehyde (180 g, 1.0 eq.). After the addition was complete, the ice bath was removed, and the reaction was allowed to stir for 5 h at 25° C. The mixture was poured into ice (5 L), filtered and then dried under vacuum. The title compound was obtained as a yellow solid (219 g).

¹H NMR (400 MHz, CDCl₃) δ 10.39 (s, 1H), 8.23 (d, J=6.8 Hz, 1H), 7.91 (d, J=7.6 Hz, 1H).

### 3.2: tert-butyl (2-fluoro-5-formyl-4-nitrophenyl)carbamate (Compound 3.2)

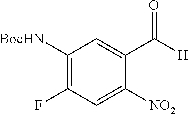 [SMILES: CC(C)(C)OC(=O)Nc1cc(C=O)c([N+](=O)[O-])cc1F]

A mixture of Compound 3.1 (219 g, 1.0 eq., tert-butyl carbamate (124 g, 1.2 eq.), Cs₂CO₃ (575 g, 2.0 eq.), Pd₂(dba)₃ (40 g, 0.05 eq.) and XPhos (84 g, 0.2 eq.) in toluene (2000 mL) was degassed and purged with N₂ for three cycles. The mixture was then stirred at 90° C. for 15 h under N₂ atmosphere. The reaction mixture was diluted with H₂O (800 mL) and extracted with EtOAc (300 mL×2). The combined organic layers were washed with brine (200 mL×2), then dried over sodium sulfate, filtered and concentrated under reduced pressure. The residue was purified by column chromatography (SiO₂, petroleum ether: ethyl acetate=100:1 to 20:1) to afford the title compound as a yellow solid (140 g, 56% yield).

¹H NMR (400 MHz, DMSO-d6) δ 10.24 (s, 1H), 9.94 (s, 1H), 8.42 (d, J=7.6 Hz, 1H), 8.16 (d, J=10.8 Hz, 1H), 1.50 (s, 9H)

### 3.3: tert-butyl (4-amino-2-fluoro-5-formylphenyl)carbamate (Compound 3.3)

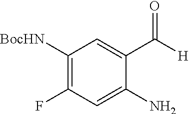 [SMILES: CC(C)(C)OC(=O)Nc1cc(C=O)c(N)cc1F]

To a solution of Compound 3.2 (100 g, 1.0 eq.) in H₂O (300 mL) and EtOH (1200 mL) was added NH₄Cl (30.5 g, 1.62 eq.). Iron (78.6 g, 4.0 eq.) was added in portions at 80° C. The mixture was stirred at 80° C. for 6 h. The mixture was filtered, water was added to the filtrate, and the resulting mixture was extracted with ethyl acetate. The organic layer was washed with brine, dried over sodium sulfate, and concentrated under vacuum. The residue was purified by column chromatography (SiO₂, Petroleum ether: ethyl acetate=1:0 to 0:1), TLC (petroleum ether) to afford the title compound as a yellow solid (19.0 g, 21% yield).

LC/MS: Calc'd m/z=254.1 for C₁₂H₁₅FN₂O₃, found [M+H]⁺=255.0.

¹H NMR (400 MHz, DMSO-d6) δ 9.73 (s, 1H), 8.57 (s, 1H), 7.58 (d, J=4.8 Hz, 1H), 7.21 (s, 2H), 6.53 (d, J=12.8 Hz, 1H), 1.43 (s, 9H).

### 3.4: tert-butyl (S)-(4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.4)

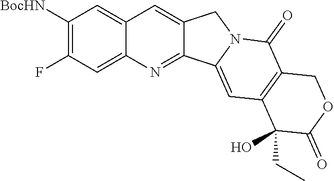 [SMILES: CC[C@@]1(O)C(=O)OCc2c1cc1n(c2=O)Cc2cc3cc(NC(=O)OC(C)(C)C)c(F)cc3nc2-1]

A mixture of Compound 3.3 (4.20 g, 1.2 eq.), (S)-4-ethyl-4-hydroxy-7,8-dihydro-1H-pyrano[3,4-f]indolizine-3,6,10(4H)-trione (3.5 g, 1 eq.) and TsOH (monohydrate, 253 mg, 0.1 eq.) in toluene (350 mL) was stirred at 110° C. for 2 hrs. The reaction solution was cooled to 25° C., filtered, the solid was washed with methyl-t-butyl ether (30 mL) and then dried under vacuum. The title compound was obtained as a yellow solid (4.5 g, 62% yield).

LC/MS: Calc'd m/z=481.2 for C₂₅H₂₄FN₃O₆, found [M+H]⁺=482.1.

¹H NMR (400 MHz, DMSO-d6) δ 9.49 (s, 1H), 8.65 (s, 1H), 8.43 (d, J=8.4 Hz, 1H), 7.95 (d, J=12.0 Hz, 1H), 7.30 (s, 1H), 6.51 (s, 1H), 5.42 (s, 2H), 5.25 (s, 2H), 1.80-1.92 (m, 2H),

### 1.52 (s, 9H), 0.88 (t, J=7.2 Hz, 3H)

### 3.5: tert-butyl (S)-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)carbamate (Compound 3.5)

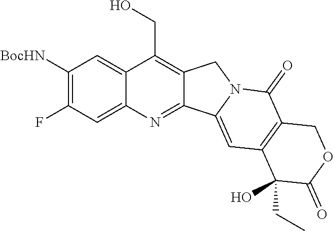 [SMILES: CC[C@@]1(O)C(=O)OCc2c1cc1n(c2=O)Cc2c-1nc1cc(F)c(NC(=O)OC(C)(C)C)cc1c2CO]

To a mixture of Compound 3.4 (4.00 g) in MeOH (360 mL) was added a solution of FeSO₄ (heptahydrate, 1.2 g), H₂SO₄ (280 μL) in H₂O (4 mL). The reaction mixture was heated at 65° C. while H₂O₂ (24 mL, 30% purity) was added dropwise over 30 min and then stirred 0.5 h. The reaction solution was cooled to 25° C., then filtered to provide the title compound as a yellow solid (1.53 g, 33.2% yield). To the filtrate was added H₂O (400 mL), then quenched with saturated aqueous Na₂S₂O₃. The pH was adjusted to 7-8 with saturated aqueous Na₂CO₃ then the solution was concentrated and filtered. The solid was triturated with MeOH (30 mL) at 55° C. for 1 h, then filtered, to provide a second batch of the title compound as a brown solid (1.09 g, 26% yield).

LC/MS: Calc'd m/z=511.2 for C₂₆H₂₆FN₃O₇, found [M+H]⁺=512.2.

¹H NMR (300 MHz, d6-DMSO) δ 9.47 (s, 1H), 8.47 (d, J=7.6 Hz, 1H), 7.94 (d, J=12.0 Hz, 1H), 7.29 (d, J=1.6 Hz, 1H), 6.49 (s, 1H), 5.86-5.76 (m, 1H), 5.42 (s, 2H), 5.38 (s, 2H), 5.16 (d, J=4.4 Hz, 2H), 1.90-1.83 (m, 2H), 1.52 (s, 9H), 0.88 (t, J=6.4 Hz, 3H).

### 3.6: tert-butyl(S)-(4-ethyl-8-fluoro-11-formyl-4-hydroxy-3,14-dioxo-3,4,12,14-tetrahydro-1H-pyrano[3′,4′:6,7]indolizino[1,2-b]quinolin-9-yl)carbamate (Compound 3.6)

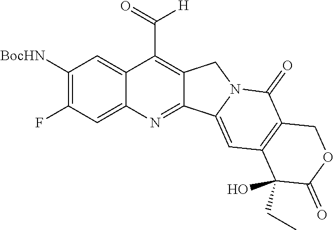 [SMILES: CC[C@@]1(O)C(=O)OCc2c1cc1n(c2=O)Cc2c-1nc1cc(F)c(NC(=O)OC(C)(C)C)cc1c2C=O]

In a 50 mL round-bottom as containing Compound 3.5 (150 mg, 0.293 mmol) was added DCM (2.9 mL) followed by Dess-Martin periodinane (0.56 g, 1.32 mmol) and water (15.8 μL, 0.88 mmol). This solution was stirred at room temperature for 18 h then diluted with DCM, washed with saturated aqueous NaHCO₃ and brine. The layers were separated, and the combined organic layers were evaporated onto celite. Flash purification was accomplished as described in General Procedure 9, using a 10 g silica column and eluting with 0 to 10% DCM/MeOH to give the title product as an orange powder (42.5 mg, 28%).

LC/MS: Calc'd m/z=509.2 for C₂₆H₂₄FN₃O₇, found [M+H]⁺=510.4.

¹H NMR (300 MHz, Acetone-d6) δ 11.10 (s, 1H), 9.68 (d, J=8.6 Hz, 1H), 8.81 (s, 1H), 8.04 (d, J=11.9 Hz, 1H), 7.63 (s, 1H), 5.73 (s, 2H), 5.69 (d, J=16.2 Hz, 1H), 5.42 (d, J=16.2 Hz, 1H), 2.02-1.95 (m, 2H), 8.47 (d, J=7.6 Hz, 1H), 7.94 (d, J=12.0 Hz, 1H), 7.29 (d, J=1.6 Hz, 1H), 6.49 (s, 1H), 5.86-5.76 (m, 1H), 5.42 (s, 2H), 5.38 (s, 2H), 5.16 (d, J=4.4 Hz, 2H), 1.90-1.83 (m, 2H), 1.52 (s, 9H), 0.88 (t, J=6.4 Hz, 3H).

### 3.7: (S)-9-amino-4-ethyl-8-fluoro-4-hydroxy-1,12-dihydro-14H-pyrano[3′,4′:6,7]indolizino [1,2-b]quinoline-3,14(4H)-dione (Compound 140)

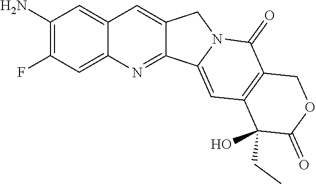 [SMILES: CC[C@@]1(O)C(=O)OCc2c1cc1n(c2=O)Cc2cc3cc(N)c(F)cc3nc2-1]

The title compound was prepared according to General Procedure 6 starting from Compound 3.4 (40 mg) to give the title compound as a red solid (TFA salt, 36 mg, 87% yield).

LC/MS: Calc'd m/z=381.1 for C₂₀H₁₆FN₃O₄, found [M+H]⁺=382.2.

¹H NMR (300 MHz, DMSO) δ 8.28 (s, 1H), 7.72 (d, J=12.5 Hz, 1H), 7.21 (d, J=7.3 Hz, 1H), 5.43 (d, J=16.2 Hz, 1H), 5.34 (d, J=16.2 Hz, 1H), 5.17 (s, 2H), 1.92-1.74 (m, 2H), 0.88 (t, J=7.3 Hz, 3H).

### 3.8: (S)-9-amino-4-ethyl-8-fluoro-4-hydroxy-11-(hydroxymethyl)-1,12-dihydro-14H-pyrano[3′,4′:6,7]indolizino[1,2-b]quinoline-3,14(4H)-dione (Compound 141)

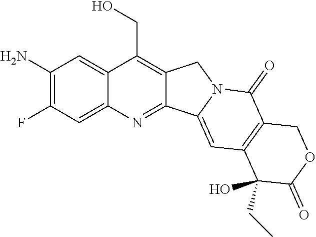 [SMILES: CC[C@@]1(O)C(=O)OCc2c1cc1n(c2=O)Cc2c-1nc1cc(F)c(N)cc1c2CO]

The title compound was prepared according to General Procedure 6 starting from Compound 3.5 (5 mg) to give the title compound as a red solid (TFA salt, 4.1 mg, 78% yield).

LC/MS: Calc'd m/z=411.2 for C₂₁H₁₈FN₃O₅, found [M+H]⁺=412.2.

¹H NMR (300 MHz, MeOD) δ 7.71 (d, J=12.2 Hz, 1H), 7.60 (s, 1H), 7.29 (d, J=9.5 Hz, 1H), 5.61 (d, J=16.3 Hz, 1H), 5.47 (s, 2H), 5.40 (d, J=16.3 Hz, 1H), 5.25 (s, 2H), 2.03-1.94 (m, 2H), 1.03 (t, J=7.4 Hz, 3H).

### 3.9: tert-butyl (S)-(11-(chloromethyl)-4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.9)

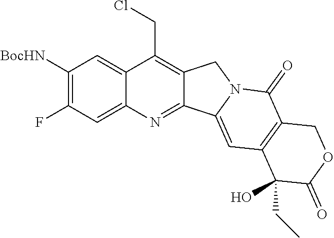 [SMILES: CC[C@@]1(O)C(=O)OCc2c1cc1n(c2=O)Cc2c-1nc1cc(F)c(NC(=O)OC(C)(C)C)cc1c2CCl]

To a stirring solution of Compound 3.5 (100 mg) in dichloromethane (5 mL) was added a solution of thionyl chloride (14 μL) in dichloromethane (0.1 mL). After 1 h, additional thionyl chloride (14 μL) in dichloromethane (0.1 mL) was added. After another 1 h the reaction was diluted with dichloromethane (10 mL) and toluene (1 mL) then concentrated in vacuo to provide the title compound as a red solid that was used in subsequent reactions without additional purification.

LC/MS: Calc'd m/z=529.1 for C₂₆H₂₅C₁FN₃O₆, found [M+H]⁺=530.2.

### 3.10: tert-butyl (S)-(11-(aminomethyl)-4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.10)

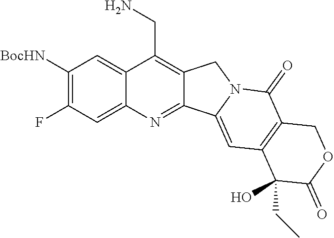 [SMILES: CC[C@@]1(O)C(=O)OCc2c1cc1n(c2=O)Cc2c-1nc1cc(F)c(NC(=O)OC(C)(C)C)cc1c2CN]

To Compound 3.9 (100 mg) in ethanol (500 μL) was added hexamethylenetetramine (79 mg) then DIPEA (99 μL). This solution was heated at 60° C. for 16 h then concentrated to dryness in vacuo. Flash purification was accomplished as described in General Procedure 9, using a 12 g C18 column and eluting with a 10 to 50% CH₃CN/H₂O+0.1% TFA gradient to give the title compound as an off-white solid (TFA salt, 29 mg, 24% yield).

LC/MS: Calc'd m/z=510.2 for C₂₆H₂₇FN₄O₆, found [M+H]⁺=511.4.

¹H NMR (300 MHz, MeOD) δ 8.88 (d, J=8.2 Hz, 1H), 7.96 (d, J=11.9 Hz, 1H), 7.62 (s, 1H), 5.60 (d, J=16.4 Hz, 1H), 5.48 (s, 2H), 5.41 (d, J=16.4 Hz, 1H), 4.80 (s, 2H), 2.07-1.89 (m, 2H), 1.64 (s, 9H), 1.02 (t, J=7.3 Hz, 3H).

### 3.11: (S)-9-amino-11-(aminomethyl)-4-ethyl-8-fluoro-4-hydroxy-1,12-dihydro-14H-pyrano[3′,4′:6,7]indolizino[1,2-b]quinoline-3,14(4H)-dione (Compound 145)

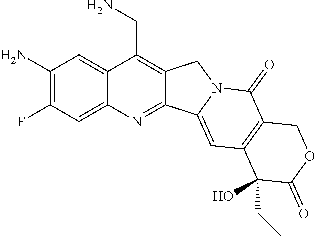 [SMILES: CC[C@@]1(O)C(=O)OCc2c1cc1n(c2=O)Cc2c-1nc1cc(F)c(N)cc1c2CN]

The title compound was prepared according to General Procedure 6 starting from Compound 3.10 (2.1 mg) to give the title compound as a red solid (TFA salt, 1.8 mg, 100% yield).

LC/MS. Calc'd m/z=410.1 for C₂₁H₁₉FN₄O₄, found [M+H]⁺=411.2.

¹H NMR (300 MHz, MeOD) δ 7.82 (d, J=12.1 Hz, 1H), 7.60 (s, 1H), 7.37 (d, J=9.1 Hz, 1H), 5.61 (d, J=16.3 Hz, 1H), 5.42 (s, 2H), 5.41 (d, J=16.3 Hz, 1H), 4.69 (s, 2H), 2.08-1.94 (m, 2H), 1.03 (t, J=7.4 Hz, 3H).

### Example 3.12: (S)-9-amino-4-ethyl-8-fluoro-4-hydroxy-11-(morpholinomethyl)-1,12-dihydro-14H-pyrano[3′,4′:6,7]indolizino[1,2-b]quinoline-3,14(4H)-dione (Compound 3.12)

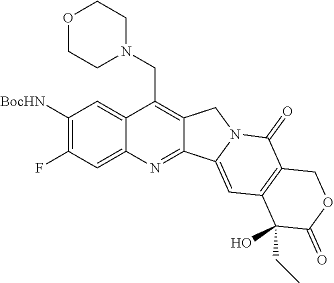 [SMILES: CC[C@@]1(O)C(=O)OCc2c1cc1n(c2=O)Cc2c-1nc1cc(F)c(NC(=O)OC(C)(C)C)cc1c2CN1CCOCC1]

The title compound was prepared according to General Procedure 1 starting from Compound 3.9 (150 mg) and morpholine. Preparative HPLC purification was accomplished as described in General Procedure 9, eluting with a 10 to 60% CH₃CN/H₂O+0.1% TFA gradient to give the title compound as a red solid (TFA salt, 103 mg, 52% yield).

LC/MS: Calc'd m/z=580.2 for C₃₀H₃₃FN₄O₇, found [M+H]⁺=581.4.

¹H NMR (300 MHz, MeOD) δ 9.06 (d, J=8.3 Hz, 1H), 7.93 (d, J=12.0 Hz, 1H), 7.66 (s, 1H), 5.63 (d, J=16.3 Hz, 1H), 5.51 (s, 2H), 5.43 (d, J=16.4 Hz, 1H), 4.92 (s, 2H), 3.84 (s, 4H), 3.10 (s, 4H), 1.99 (d, J=5.5 Hz, 2H), 1.63 (s, 9H), 1.03 (t, J=7.4 Hz, 3H).

### 3.13: (S)-9-amino-4-ethyl-8-fluoro-4-hydroxy-11-(morpholinomethyl)-1,12-dihydro-14H-pyrano[3′,4′:6,7]indolizino[1,2-b]quinoline-3,14(4H)-dione (Compound 142)

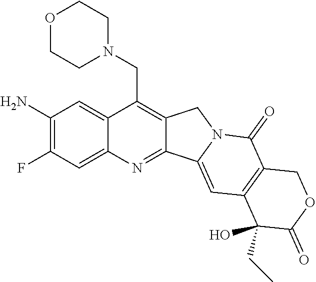 [SMILES: CC[C@@]1(O)C(=O)OCc2c1cc1n(c2=O)Cc2c-1nc1cc(F)c(N)cc1c2CN1CCOCC1]

The title compound was prepared according to General Procedure 6 starting from Compound 3.12 (45 mg) to give the title compound as a red solid (TFA salt, 37 mg, 99% yield).

LC/MS: Calc'd m/z=480.2 for C₂₅H₂₅FN₄O₅, found [M+H]⁺=481.4.

¹H NMR (300 MHz, MeOD) δ 7.73 (d, J=12.0 Hz, 1H), 7.54 (s, 1H), 7.48 (d, J=9.2 Hz, 1H), 5.60 (d, J=16.3 Hz, 1H), 5.47-5.34 (m, 3H), 4.65 (s, 2H), 3.91-3.85 (m, 4H), 3.30-3.24 (m, 4H), 2.08-1.91 (m, 2H), 1.02 (t, J=7.3 Hz, 3H).

### 3.14: (S)-9-amino-4-ethyl-8-fluoro-4-hydroxy-11-(piperidin-1-ylmethyl)-1,12-dihydro-14H-pyrano[3′,4′:6,7]indolizino[1,2-b]quinoline-3,14(4H)-dione (Compound 148)

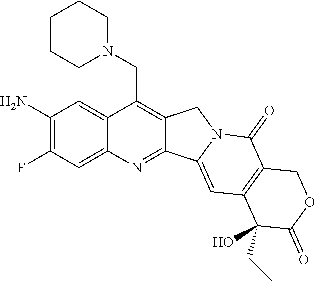 [SMILES: CC[C@@]1(O)C(=O)OCc2c1cc1n(c2=O)Cc2c-1nc1cc(F)c(N)cc1c2CN1CCCCC1]

To a 5 mL flask containing Compound 3.6 (37 mg, 0.067 mmol) was added dichloromethane (1.45 mL) followed by acetic acid (18.69 μL, 0.327 mmol), piperidine (21.52 μL, 0.218 mmol), and sodium triacetoxyborohydride (23.0 mg, 0.109 mmol). This solution was then stirred at room temperature for 2 h, quenched by the addition of water+0.1% TFA and DMF (1:1, 1.0 mL), and partially evaporated. Purification was accomplished as described in General Procedure 9, using a 12 g C18 flash column and eluting with a 5 to 40% CH₃CN/H₂O+0.1% TFA gradient to give the Boc-protected intermediate as a yellow powder. This intermediate was then deprotected according to General Procedure 6 to give the title compound as a yellow solid (TFA salt, 32.5 mg, 98% yield).

LC/MS: Calc'd m/z=478.2 for C₂₆H₂₇FN₄O₄, found [M+H]⁺=479.4.

¹H NMR (300 MHz, MeOD) δ 7.78 (d, J=12.1 Hz, 1H), 7.56 (s, 1H), 7.41 (d, J=9.1 Hz, 1H), 5.60 (d, J=16.4 Hz, 1H), 5.47-5.35 (m, 3H), 4.86 (s, 2H), 3.80-3.68 (m, 2H), 3.28-3.19 (m, 2H), 2.02-1.68 (m, 8H), 1.01 (t, J=7.4 Hz, 3H).

### 3.15: (S)-9-amino-4-ethyl-8-fluoro-4-hydroxy-11-((4-methylpiperazin-1-yl)methyl)-1,12-dihydro-14H-pyrano[3′,4′:6,7]indolizino[1,2-b]quinoline-3,14(4H)-dione (Compound 149)

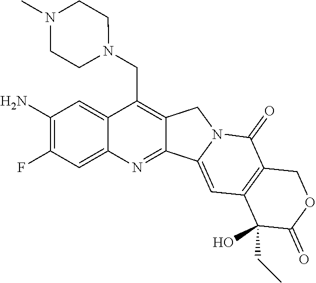 [SMILES: CC[C@@]1(O)C(=O)OCc2c1cc1n(c2=O)Cc2c-1nc1cc(F)c(N)cc1c2CN1CCN(C)CC1]

To a 2 mL vial containing Compound 3.6 (15 mg, 0.029 mmol) was added dichloromethane (0.59 mL), acetic acid (7.58 μL, 0.132 mmol), and N-methylpiperazine (4.90 μL, 0.044 mmol). This solution was stirred at room temperature for 4 h then sodium triacetoxyborohydride (7.8 mg, 0.037 mmol) was then added and stirred for an additional 45 min. Excess hydride was then quenched by the addition of a 0.1% aqueous TFA solution (0.5 mL). Purification was accomplished as described in General Procedure 9 using a 12 g C18 flash column and eluting with a 5 to 40% CH₃CN/H₂O+0.1% TFA gradient to give the Boc-protected intermediate as a yellow powder. This intermediate was deprotected according to General Procedure 6 to give the title product as a yellow solid (TFA salt, 1.5 mg, 7.1% yield).

LC/MS: Calc'd m/z=493.2 for C₂₆H₂₈FN₅O₄, found [M+H]⁺=494.4.

¹H NMR (300 MHz, MeOD) δ 7.68 (d, J=12.2 Hz, 1H), 7.56 (s, 1H), 7.53 (d, J=9.5 Hz, 1H), 5.60 (d, J=16.3 Hz, 1H), 5.45-5.30 (m, 3H), 4.15 (s, 2H), 3.55-3.44 (m, 2H), 3.18-3.07 (m, 2H), 2.93 (s, 3H), 2.70-2.51 (m, 2H), 2.03-1.89 (m, 2H), 1.02 (t, J=7.4 Hz, 3H).

### 3.16: (S)-9-amino-4-ethyl-8-fluoro-4-hydroxy-11-((4-(phenylsulfonyl)piperazin-1-yl)methyl)-1,12-dihydro-14H-pyrano[3′,4′:6,7]indolizino[1,2-b]quinoline-3,14(4H)-dione (Compound 153)

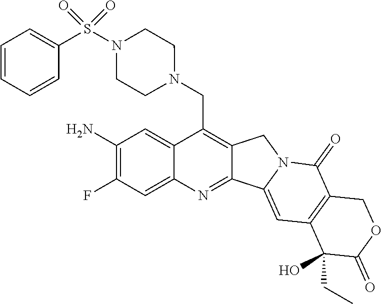 [SMILES: CC[C@@]1(O)C(=O)OCc2c1cc1n(c2=O)Cc2c-1nc1cc(F)c(N)cc1c2CN1CCN(S(=O)(=O)c2ccccc2)CC1]

The Boc-protected precursor of the title compound was prepared according to General Procedure 1 starting from Compound 3.9 (10 mg) and 1-(phenylsulfonyl)piperazine. Preparative HPLC was accomplished as described in General Procedure 9, eluting with a 35 to 44% CH₃CN/H₂O+0.1% TFA gradient to give the Boc-protected intermediate as a yellow powder. This intermediate was then deprotected according to General Procedure 6 to give the title compound (TFA salt, 2.4 mg, 17% yield over 2 steps).

LC/MS: Calc'd m/z=619.2 for C₃₁H₃₀FN₅O₆S, found [M+H]⁺=520.4.

¹H NMR (300 MHz, MeOD) δ 7.81-7.60 (m, 7H), 7.34 (s, 1H), 5.51 (d, J=16.4 Hz, 1H), 5.35 (d, J=16.4 Hz, 1H), 5.22 (s, 2H), 4.10 (s, 2H), 3.15-3.02 (m, 4H), 2.79-2.71 (m, 4H), 2.00-1.93 (m, 2H), 1.00 (t, J=7.4 Hz, 3H).

### 3.17: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)acetamide (Compound 147)

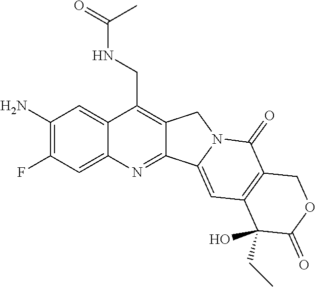 [SMILES: CC[C@@]1(O)C(=O)OCc2c1cc1n(c2=O)Cc2c-1nc1cc(F)c(N)cc1c2CNC(C)=O]

The title compound was prepared according to General Procedure 2 followed by General Procedure 6 starting from Compound 3.10 (8 mg) and acetic acid. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (4.0 mg, 56% yield).

LC/MS: Calc'd m/z=452.2 for C₂₃H₂₁FN₄O₅, found [M+H]⁺=453.2.

¹H NMR (300 MHz, MeOD) δ 7.69 (d, J=12.1 Hz, 1H), 7.56 (s, 1H), 7.38 (d, J=9.3 Hz, 1H), 5.59 (d, J=16.3 Hz, 1H), 5.44-5.33 (m, 3H), 4.85 (s, 3H), 2.03 (s, 3H), 2.00-1.84 (m, 2H), 1.03 (t, J=7.4 Hz, 3H).

### 3.18: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)methanesulfonamide (Compound 146)

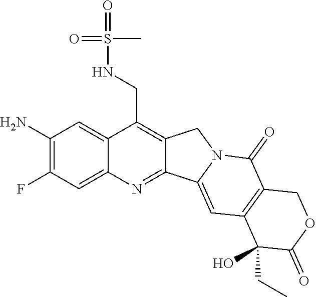 [SMILES: CC[C@@]1(O)C(=O)OCc2c1cc1n(c2=O)Cc2c-1nc1cc(F)c(N)cc1c2CNS(C)(=O)=O]

The title compound was prepared according to General Procedure 3 followed by General Procedure 6 starting from Compound 3.10 (8 mg) and methane sulfonyl chloride. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (4.4 mg, 57% yield).

LC/MS: Calc'd m/z=488.1 for C₂₂H₂₁FN₄O₆S, found [M+H]⁺=489.2.

¹H NMR (300 MHz, MeOD) δ 7.74 (d, J=12.2 Hz, 1H), 7.60 (s, 1H), 7.49 (d, J=9.3 Hz, 1H), 5.61 (d, J=16.2 Hz, 1H), 5.45 (s, 2H), 5.40 (d, J=16.2 Hz, 1H), 4.78 (s, 2H), 3.05 (s, 3H), 2.08-1.94 (m, 2H), 1.03 (t, J=7.4 Hz, 3H).

### 3.19: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-2-hydroxyethane-1-sulfonamide (Compound 150)

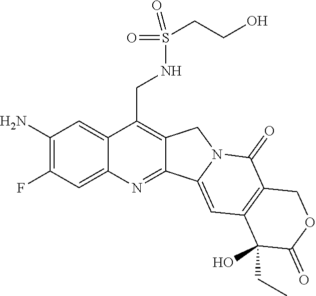 [SMILES: CC[C@@]1(O)C(=O)OCc2c1cc1n(c2=O)Cc2c-1nc1cc(F)c(N)cc1c2CNS(=O)(=O)CCO]

The title compound was prepared according to General Procedure 3 followed by General Procedure 6 starting from Compound 3.10 (6 mg) and 2-hydroxyethanesulfonyl chloride. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (1 mg, 16% yield).

LC/MS: Calc'd m/z=518.5 for C₂₃H₂₃FN₄O₇S, found [M+H]⁺=519.5.

¹H NMR (300 MHz, 10% D₂O/CD₃CN) δ 7.77-7.61 (m, 1H), 7.48-7.30 (m, 2H), 5.53 (d, J=16.3 Hz, 1H), 5.31 (d, J=15.4 Hz, 3H), 4.69 (s, 2H), 3.97 (dd, J=6.6, 4.9 Hz, 2H), 3.39 (t, J=5.8 Hz, 2H), 2.93 (s, 1H), 1.99-1.83 (m, 2H), 0.94 (t, J=7.3 Hz, 3H).

### 3.20: 4-nitrophenyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 3.20)

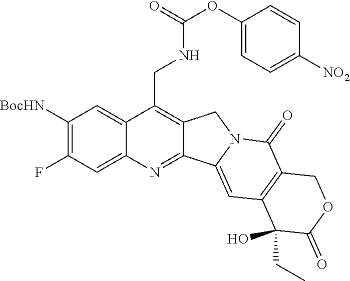 [SMILES: CC[C@@]1(O)C(=O)OCc2c1cc1n(c2=O)Cc2c-1nc1cc(F)c(NC(=O)OC(C)(C)C)cc1c2CNC(=O)Oc1ccc([N+](=O)[O-])cc1]

To a solution of Compound 3.10 (10 mg, 0.02 mmol) in DMF (400 μL, 0.05 M) was added 4-nitrophenyl carbonate (12 mg, 0.04 mmol) and diisopropylethylamine (6.8 μL, 0.04 mmol). This solution was stirred at room temperature for ~30 min, then used directly in subsequent reactions.

### 3.21: Methyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 143)

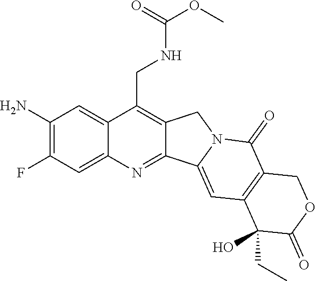 [SMILES: CC[C@@]1(O)C(=O)OCc2c1cc1n(c2=O)Cc2c-1nc1cc(F)c(N)cc1c2CNC(=O)OC]

The title compound was prepared by addition of MeOH (100 μL) to 200 ul of the solution of Compound 3.20. This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (2.1 mg, 47% yield).

LC/MS: Calc'd m/z=468.4 for C₂₃H₂₁FN₄O₆, found [M+H]⁺=468.3.

¹H NMR (300 MHz, 10% D₂O/CD₃CN) δ 7.72 (d, J=12.2 Hz, 1H), 7.41 (d, J=18.1 Hz, 1H), 6.96 (s, 1H), 5.52 (d, J=3.6 Hz, 1H), 5.39-5.23 (m, 3H), 4.82 (s, 1H), 4.73 (s, 1H), 3.63 (d, J=1.2 Hz, 3H), 1.56 (s, 3H), 1.27 (s, 2H), 0.94 (t, J=7.4 Hz, 3H).

### 3.22: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-methylurea (Compound 144)

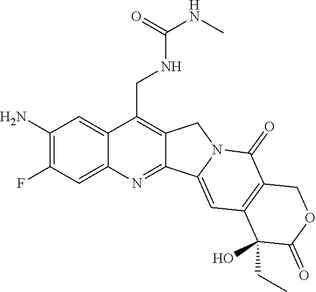 [SMILES: CC[C@@]1(O)C(=O)OCc2c1cc1n(c2=O)Cc2c-1nc1cc(F)c(N)cc1c2CNC(=O)NC]

The title compound was prepared by addition of methylamine hydrochloride (10 mg) to 200 ul of the solution of Compound 3.20, followed by iPr₂NEt (5 μL). This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (2.9 mg, 64.5% yield).

LC/MS: Calc'd m/z=467.5 for C₂₃H₂₁FN₅O₅, found [M+H]⁺=468.5.

¹H NMR (300 MHz, 10% D₂O/CD₃CN) δ 8.13 (d, J=9.2 Hz, 1H), 7.92 (s, 1H), 7.73 (d, J=12.3 Hz, 1H), 7.52-7.35 (m, 2H), 6.94 (d, J=9.2 Hz, 2H), 5.55 (d, J=16.5 Hz, 2H), 5.44-5.27 (m, 4H), 4.85 (s, 2H), 4.78 (s, 1H), 1.56 (d, J=2.5 Hz, 3H), 1.27 (s, 2H), 0.93 (q, J=11.7, 9.5 Hz, 3H).

### 3.23: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-(2-hydroxyethyl)urea (Compound 151)

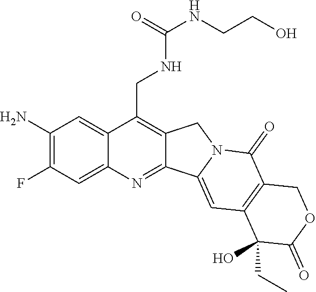 [SMILES: CC[C@@]1(O)C(=O)OCc2c1cc1n(c2=O)Cc2c-1nc1cc(F)c(N)cc1c2CNC(=O)NCCO]

The title compound was prepared by addition of ethanolamine (100 μL) to 200 ul of the solution of Compound 3.20. This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (0.5 mg, 8.5% yield).

LC/MS: Calc'd m/z=497.5 for C₂₄H₂₄FN₅O₆, found [M+H]⁺=498.5.

¹H NMR (300 MHz, 10% D₂O/CD₃CN) δ 7.77-7.61 (m, 1H), 7.48-7.30 (m, 2H), 5.53 (d, J=16.3 Hz, 1H), 5.31 (d, J=15.4 Hz, 1H), 5.19 (s, 2H), 4.69 (s, 2H), 3.97 (dd, J=6.6, 4.9 Hz, 2H), 3.39 (t, J=5.8 Hz, 2H), 2.93 (s, 1H), 2.01-1.83 (m, 2H), 0.94 (t, J=7.3 Hz, 3H).

### 3.24: (S)-9-amino-11-(azidomethyl)-4-ethyl-8-fluoro-4-hydroxy-1,12-dihydro-14H-pyrano[3′,4′:6,7]indolizino[1,2-b]quinoline-3,14(4H)-dione (Compound 152)

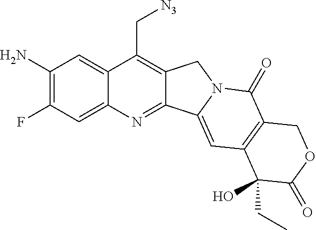 [SMILES: CC[C@@]1(O)C(=O)OCc2c1cc1n(c2=O)Cc2c-1nc1cc(F)c(N)cc1c2CN=[N+]=[N-]]

To a stirring solution of Compound 3.5 (100 mg) in 2 mL dichloromethane was added thionyl chloride (35 μL, 2.5 eq.). The solution was stirred at room temperature for 20 min, then additional thionyl chloride (35 μL, 2.5 eq.) was added. After 20 minutes, toluene (1 mL) was added, and the reaction mixture was concentrated in vacuo. The crude solid was suspended in DMSO (1 mL) and sodium azide (19 mg, 1.5 eq.) was added. This solution was stirred at room temperature for 16 h. Purification was accomplished as described in General Procedure 9, eluting with a 5 to 50% CH₃CN/H₂O+0.1% TFA gradient to give the title compound as an off-white solid (20 mg, 23% yield).

LC/MS: Calc'd m/z=436.1 for C₂₁H₁₇FN₆O₄, found [M+H]⁺=437.2.

¹H NMR (300 MHz, MeOD) δ 7.75 (d, J=12.2 Hz, 1H), 7.60 (s, 1H), 7.38 (d, J=9.3 Hz, 1H), 5.61 (d, J=16.3 Hz, 1H), 5.46-5.35 (m, 3H), 5.07 (s, 2H), 2.03-1.97 (m, 2H), 1.03 (t, J=7.3 Hz, 3H).

### 3.25: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)acetamide (Compound 164)

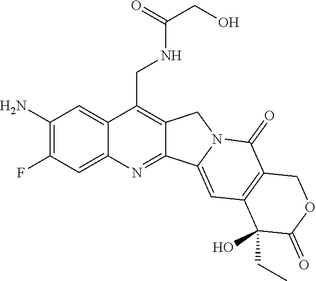 [SMILES: CC[C@@]1(O)C(=O)OCc2c1cc1n(c2=O)Cc2c-1nc1cc(F)c(N)cc1c2CNC(=O)CO]

The title compound was prepared according to General Procedure 2 starting from Compound 145 (10 mg) and glycolic acid. Preparative HPLC purification was accomplished as described in General Procedure 9, eluting with a 10 to 45% CH₃CN/H₂O+0.1% TFA gradient. The title compound was obtained as a yellow solid (6.9 mg, 60% yield).

LC/MS: Calc'd m/z=468.1 for C₂₃H₂₁FN₄O₆, found [M+H]⁺=469.2.

¹H NMR (300 MHz, MeOD) 7.70 (d, J=12.2 Hz, 1H), 7.60 (s, 1H), 7.42 (d, J=9.4 Hz, 1H), 5.62 (d, J=16.3 Hz, 1H), 5.43 (s, 2H), 5.36 (d, J=16.2 Hz, 1H), 4.95 (d, J=5.9 Hz, 2H), 4.08 (s, 2H), 2.04-1.90 (m, 1H), 1.03 (t, J=7.4 Hz, 3H).

### 3.26: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-methylthiourea (Compound 161)

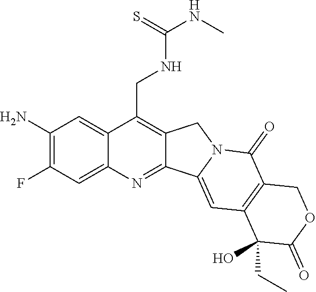 [SMILES: CC[C@@]1(O)C(=O)OCc2c1cc1n(c2=O)Cc2c-1nc1cc(F)c(N)cc1c2CNC(=S)NC]

To a solution of Compound 145 (9 mg, 1.0 eq.) in DMF (1 mL) was added thiocarbonyldiimidazole (6 mg, 1.5 eq.) then DIPEA (8 μL, 2.0 eq.). The resulting solution was stirred at 25° C. for 2 h, after which complete conversion to the isothiocyanate intermediate was observed. Methylammonium chloride (3 mg, 2.0 eq.) was then added and the reaction mixture was heated at 60° C. for 30 min. Preparative HPLC purification was accomplished as described in General Procedure 9, eluting with a 10 to 45% CH₃CN/H₂O+0.1% TFA gradient. The title compound was obtained as a yellow solid (2.3 mg, 22% yield).

LC/MS: Calc'd m/z=483.1 for C₂₃H₂₂FN₅O₄S found [M+H]⁺=484.2.

¹H NMR (300 MHz, MeOD) δ 7.70 (d, J=12.0 Hz, 1H), 7.60 (s, 1H), 7.38 (d, J=9.3 Hz, 1H), 5.62 (d, J=16.2 Hz, 1H), 5.36 (s, 2H), 5.31 (d, J=16.2 Hz, 1H), 5.30 (s, 2H), 3.04 (s, 3H), 1.99-1.90 (m, 2H), 1.02 (t, J=7.4 Hz, 3H).

### 3.27: S-(2-hydroxyethyl)-(S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamothioate (Compound 160)

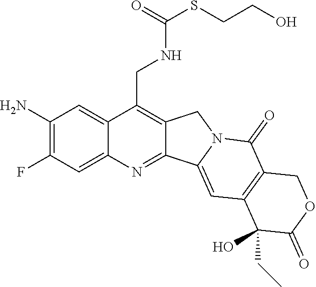 [SMILES: CC[C@@]1(O)C(=O)OCc2c1cc1n(c2=O)Cc2c-1nc1cc(F)c(N)cc1c2CNC(=O)SCCO]

The title compound was prepared according to General Procedure 5 starting from Compound 145 (10 mg) and 2-mercaptoethanol. Preparative HPLC purification was accomplished as described in General Procedure 9, eluting with a 10 to 45% CH₃CN/H₂O+0.1% TFA gradient. The title compound was obtained as a yellow solid (4.2 mg, 43% yield).

LC/MS: Calc'd m/z=514.1 for C₂₄H₂₃FN₄O₆S found [M+H]⁺=515.2.

¹H NMR (300 MHz, MeOD) δ 7.71 (d, J=12.1 Hz, 1H), 7.60 (s, 1H), 7.36 (d, J=9.4 Hz, 1H), 5.62 (d, J=16.3 Hz, 1H), 5.42 (s, 2H), 5.35 (d, J=16.2 Hz, 1H), 4.88 (d, J=4.6 Hz, 2H), 3.68 (t, J=6.4 Hz, 2H), 3.03 (t, J=6.5 Hz, 2H), 2.04-1.92 (m, 2H), 1.03 (t, J=7.4 Hz, 3H).

### 3.28: (S)-9-amino-4,11-diethyl-8-fluoro-4-hydroxy-1,12-dihydro-14H-pyrano[3′,4′:6,7]indolizino[1,2-b]quinoline-3,14(4H)-dione (Compound 154)

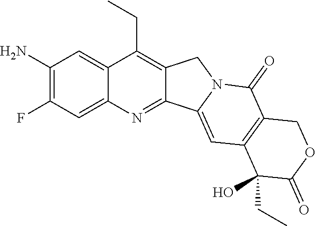 [SMILES: CCc1c2c(nc3cc(F)c(N)cc13)-c1cc3c(c(=O)n1C2)COC(=O)[C@]3(O)CC]

To a 5 mL flask containing Compound 140 (50 mg) was added water (0.72 mL), FeSO₄ (heptahydrate, 11.0 mg) and propionaldehyde (74 μL). The obtained suspension was cooled to -15° C. using an ice brine bath, then sulfuric acid (0.40 mL) was added dropwise. Hydrogen peroxide (95 μL) was then added dropwise. This mixture was stirred at −15° C. for 10 min then allowed to warm up to room temperature and stirred for 2 h. The reaction mixture was diluted with water (30 mL) and the obtained suspension was extracted with DCM (3×30 mL). The organic phase was then evaporated to dryness. Preparative HPLC purification was accomplished as described in General Procedure 9, eluting with a 25 to 70% CH₃CN/H₂O+0.1% TFA gradient to give the title compound as a dark orange solid (2.4 mg, 4.4% yield).

LC/MS: Calc'd m/z=410.1 for C₂₂H₂₀FN₃O₄ found [M+H]⁺=410.2.

¹H NMR (300 MHz, MeOD) δ 7.63 (d, J=12.3 Hz, 1H), 7.55 (s, 1H), 7.36 (d, J=9.4 Hz, 1H), 5.57 (d, J=16.4 Hz, 1H), 5.37 (d, J=16.4 Hz, 1H), 5.21 (s, 2H), 3.13 (q, J=7.7 Hz, 2H), 2.02-1.90 (m, 2H), 1.38 (t, J=7.7 Hz, 3H), 1.01 (t, J=7.3 Hz, 3H).

### 3.29: tert-butyl-(S)-(11-((carbamoyloxy)methyl)-4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.29)

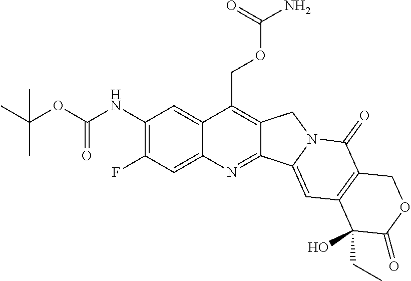 [SMILES: CC[C@@]1(O)C(=O)OCc2c1cc1n(c2=O)Cc2c-1nc1cc(F)c(NC(=O)OC(C)(C)C)cc1c2COC(N)=O]

In a 5 mL conical flask containing a solution of chlorosulfonyl isocyanate (7.7 μL) in dimethylformamide (0.29 mL), at −20° C., was added Compound 3.5 (15 mg). The obtained suspension was stirred at −20° C. for 5 min. Water (59 μL) was added, and the reaction mixture was allowed to warm up to room temperature and stirred for 2 h, then heated at 70° C. for 1 h. The reaction mixture was allowed to cool down to room temperature and partially evaporated. Preparative HPLC purification was accomplished as described in General Procedure 9, eluting with a 40 to 55% CH₃CN/H₂O+0.1% TFA gradient to give the title compound as a dark orange solid (5.1 mg, 31% yield).

LC/MS: Calc'd m/z=555.2 for C₂₇H₂₇FN₄O₈ found [M+H]⁺=555.2.

¹H NMR (300 MHz, DMSO-d6) δ 9.53 (s, 1H), 8.56 (d, J=8.5 Hz, 1H), 8.00 (d, J=12.0 Hz, 1H), 7.31 (s, 1H), 7.11-6.62 (m, 2H), 6.52 (s, 1H), 5.58 (s, 2H), 5.49-5.27 (m, 4H), 1.94-1.77 (m, 2H), 1.52 (s, 9H), 1.38 (t, J=7.7 Hz, 3H), 0.87 (t, J=7.2 Hz, 3H).

### 3.30: (S)-(9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl carbamate (Compound 169)

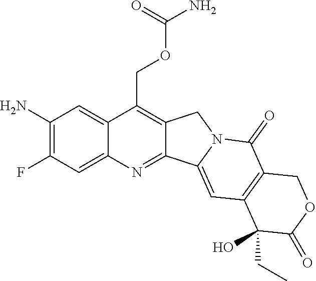 [SMILES: CC[C@@]1(O)C(=O)OCc2c1cc1n(c2=O)Cc2c-1nc1cc(F)c(N)cc1c2COC(N)=O]

The title compound was prepared according to General Procedure 6 starting from Compound 3.29 (5.1 mg) to give the title compound as yellow powder (TFA salt, 3.8 mg, 73% yield).

LC/MS: Calc'd m/z=455.1 for C₂₂H₁₉FN₄O₆ found [M+H]⁺=455.2.

¹H NMR (300 MHz, DMSO-d6) δ 7.79 (d, J=12.4 Hz, 1H), 7.29 (d, J=9.7 Hz, 1H), 7.21 (s, 1H), 7.0-6.50 (m, 2H), 5.45 (s, 2H), 5.40 (s, 2H), 5.33 (s, 2H), 1.95-1.77 (m, 2H), 0.87 (t, J=7.3 Hz, 3H).

### 3.31: ((S)-9-amino-4-ethyl-8-fluoro-4-hydroxy-11-(methoxymethyl)-1,12-dihydro-14H-pyrano[3′,4′:6,7]indolizino[1,2-b]quinoline-3,14(4H)-dione (Compound 155)

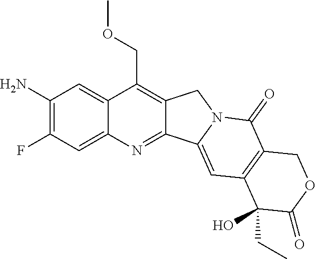 [SMILES: CC[C@@]1(O)C(=O)OCc2c1cc1n(c2=O)Cc2c-1nc1cc(F)c(N)cc1c2COC]

In a 50 mL flask containing Compound 3.5 (30 mg) was added MeOH/Dioxane (1:1) (9.8 mL) and sulfuric acid (0.73 mL). The reaction mixture was then stirred at reflux for 24 h. The reaction mixture was concentrated, poured into water (30 mL), and extracted with DCM (3×50 mL). The organic phases were combined and dried over MgSO₄. Preparative HPLC purification was accomplished as described in General Procedure 9, eluting with a 25 to 40% CH₃CN/H₂O+0.1% TFA gradient to give the title compound as a dark orange solid (5.1 mg, 16% yield).

LC/MS: Calc'd m/z=426.1 for C₂₂H₂₀FN₃O₅ found [M+H]⁺=426.2.

¹H NMR (300 MHz, DMSO-d6) δ 7.75 (d, J=12.3 Hz, 1H), 7.24 (d, J=9.9 Hz, 1H), 7.20 (s, 1H), 6.47 (s, 1H), 6.30-5.92 (brs, 2H), 5.40 (s, 2H), 5.24 (s, 2H), 4.93 (s, 2H), 3.43 (s, 3H), 1.95-1.75 (m, 2H), 0.87 (t, J=7.3 Hz, 3H).

### 3.32: (4S)-9-amino-4-ethyl-8-fluoro-4-hydroxy-11-(((1R,5S)-6-hydroxy-3-azabicyclo[3.1.1]heptan-3-yl)methyl)-1,12-dihydro-14H-pyrano[3′,4′:6,7]indolizino[1,2-b]quinoline-3,14(4H)-dione (Compound 158)

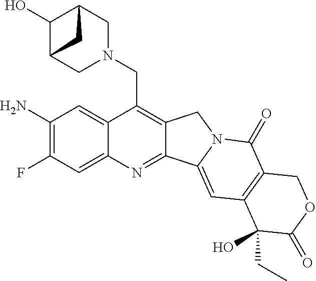 [SMILES: CC[C@@]1(O)C(=O)OCc2c1cc1n(c2=O)Cc2c-1nc1cc(F)c(N)cc1c2CN1C[C@H]2C[C@@H](C1)C2O]

In a 5 mL conical flask containing Compound 3.6 (15 mg) was added dichloromethane (0.6 mL) followed by 3-azabicyclo[3.1.1]heptan-6-ol (10 mg) and acetic acid (7.6 μL). The reaction was stirred at room temperature and sodium triacetoxyborohydride (9.4 mg) was added. After 1 hour at room temperature, the reaction was quenched by addition of water+0.1% TFA and diluted with DMF. The reaction mixture was then partially evaporated. Preparative HPLC purification was accomplished as described in General Procedure 9, eluting with a 20 to 50% CH₃CN/H₂O+0.1% TFA gradient to give the Boc-protected title compound as a yellow powder. Deprotection was performed according to General Procedure 6, and the obtained residue was purified by preparative HPLC purification as described in General Procedure 9, eluting with a 20 to 50% CH₃CN/H₂O+0.1% TFA gradient to give the title compound as yellow powder (TFA salt, 7.1 mg, 39% yield).

LC/MS: Calc'd m/z=507.2 for C₂₇H₂₇FN₄O₅ found [M+H]⁺=507.4.

¹H NMR (300 MHz, DMSO-d6) δ 7.85 (d, J=12.1 Hz, 1H), 7.46 (d, J=9.4 Hz, 1H), 7.23 (s, 1H), 6.64-5.85 (m, 3H), 5.60-5.25 (m, 4H), 4.85 (s, 1H), 4.10-3.95 (m, 1H), 3.68 (s, 2H), 2.45-2.33 (m, 2H), 1.96-1.72 (m, 2H), 0.87 (t, J=7.3 Hz, 3H).

### 3.33: (S)-9-amino-4-ethyl-8-fluoro-11-((3-fluoro-3-(hydroxymethyl)azetidin-1-yl)methyl)-4-hydroxy-1,12-dihydro-14H-pyrano[3′,4′:6,7]indolizino[1,2-b]quinoline-3,14(4H)-dione (Compound 159)

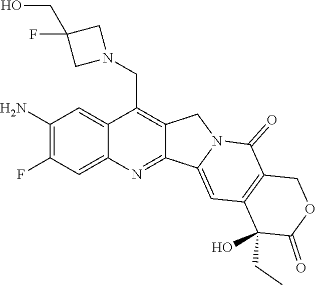 [SMILES: CC[C@@]1(O)C(=O)OCc2c1cc1n(c2=O)Cc2c-1nc1cc(F)c(N)cc1c2CN1CC(F)(CO)C1]

In a 5 mL conical flask containing Compound 3.6 (15 mg) was added dichloromethane (0.6 mL) followed by (3-fluoroazetidin-3-yl)methanol (9.3 mg) and acetic acid (7.6 μL). The reaction was stirred at room temperature and sodium triacetoxyborohydride (9.4 mg) was added. After 1 hour at room temperature, the reaction was quenched by addition of water+0.1% TFA, diluted with DMF, then partially evaporated. Preparative HPLC purification was accomplished as described in General Procedure 9, eluting with a 20 to 50% CH₃CN/H₂O+0.1% TFA gradient to give the Boc-protected title compound as a yellow powder. Deprotection was then performed according to General Procedure 6. The obtained residue was purified by preparative HPLC purification as described in General Procedure 9, eluting with a 20 to 50% CH₃CN/H₂O+0.1% TFA gradient to give the title compound as yellow powder (TFA salt, 1.8 mg, 10% yield).

LC/MS: Calc'd m/z=499.2 for C₂₅H₂₄F₂N₄O₅ found [M+H]⁺=499.4.

¹H NMR (300 MHz, DMSO-d6) δ 7.82 (d, J=12.4 Hz, 1H), 7.45 (d, J=9.5 Hz, 1H), 7.21 (s, 1H), 5.45-5.33 (m, 4H), 3.75-3.61 (m, 2H), 1.93-1.78 (m, 2H), 0.87 (t, J=7.3 Hz, 3H).

### 3.34: tert-butyl-(S)-(4-ethyl-8-fluoro-4-hydroxy-11-((methylamino)methyl)-3,14-dioxo-3,4,12,14-tetrahydro-1H-pyrano[3′,4′:6,7]indolizino[1,2-b]quinolin-9-yl)carbamate (Compound 3.34)

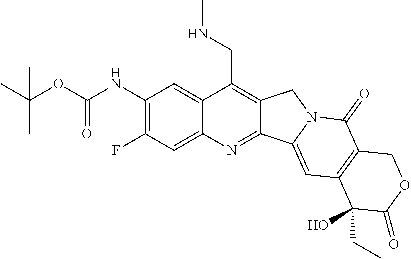 [SMILES: CC[C@@]1(O)C(=O)OCc2c1cc1n(c2=O)Cc2c-1nc1cc(F)c(NC(=O)OC(C)(C)C)cc1c2CNC]

To a stirring solution of Compound 3.9 (210 mg) in DMF (5 mL) was added sodium iodide (5.9 mg) followed by methylammonium chloride (107 mg). The reaction mixture was then stirred at room temperature overnight. Reverse phase purification was accomplished as described in General Procedure 9 using a 30 g C18 column and eluting with a 10 to 65% CH₃CN/H₂O+0.1% TFA gradient to give the title compound as a yellow solid (15.0 mg, 7.2% yield).

LC/MS: Calc'd m/z=524.2 for C₂₇H₂₉FN₄O₆, found [M+H]⁺=525.4.

### 3.35: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-2-hydroxy-N-methylacetamide (Compound 165)

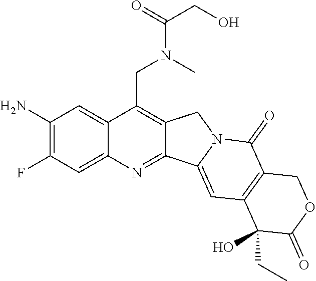 [SMILES: CC[C@@]1(O)C(=O)OCc2c1cc1n(c2=O)Cc2c-1nc1cc(F)c(N)cc1c2CN(C)C(=O)CO]

The Boc-protected version of the title compound was prepared according to General Procedure 2 starting from Compound 3.34 (6.4 mg) and glycolic acid. Preparative HPLC purification was accomplished as described in General Procedure 9, eluting with a 20 to 50% CH₃CN/H₂O+0.1% TFA gradient. Deprotection was then performed according to General Procedure 6 to give the title compound as yellow powder (TFA salt, 2.0 mg, 28% yield).

LC/MS: Calc'd m/z=482.2 for C₂₄H₂₃FN₄O₆, found [M+H]⁺=483.2.

¹H NMR (300 MHz, DMSO-d6) δ 7.79 (d, J=12.3 Hz, 1H), 7.27 (d, J=9.5 Hz, 1H), 7.22 (s, 1H), 6.48 (s, 1H), 6.28-6.02 (m, 2H), 5.40 (s, 2H), 5.21 (s, 2H), 5.06-4.93 (m, 2H), 4.18 (s, 2H), 2.80 (s, 3H), 1.92-1.78 (m, 2H), 0.87 (t, J=7.3 Hz, 3H).

### 3.36: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-N-methylmethanesulfonamide (Compound 166)

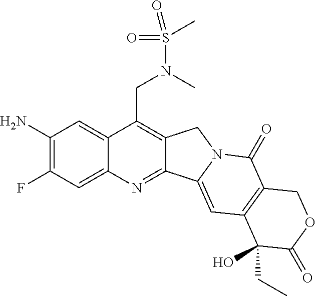 [SMILES: CC[C@@]1(O)C(=O)OCc2c1cc1n(c2=O)Cc2c-1nc1cc(F)c(N)cc1c2CN(C)S(C)(=O)=O]

The Boc-protected version of the title compound was prepared according to General Procedure 3 starting from Compound 3.34 (8.0 mg) and methanesulfonyl chloride. Preparative HPLC purification was accomplished as described in General Procedure 9, eluting with a 10 to 50% CH₃CN/H₂O+0.1% TFA gradient. Deprotection was then performed according to General Procedure 6 to give the title compound as yellow powder (TFA salt, 2.6 mg, 34% yield).

LC/MS: Calc'd m/z=502.1 for C₂₃H₂₃FN₄O₆S, found [M+H]⁺=503.2.

¹H NMR (300 MHz, DMSO-d6) δ 7.81 (d, J=12.3 Hz, 1H), 7.41 (d, J=9.4 Hz, 1H), 7.23 (s, 1H), 6.63-5.84 (m, 2H), 5.42 (s, 2H), 5.29 (s, 2H), 4.81-4.64 (m, 2H), 3.14 (s, 3H), 2.67 (s, 3H), 1.96-1.76 (m, 2H), 0.88 (t, J=7.3 Hz, 3H).

### 3.37: (S)-9-amino-4-ethyl-8-fluoro-4-hydroxy-11-(2-methoxyethyl)-1,12-dihydro-14H-pyrano[3′,4′:6,7]indolizino[1,2-b]quinoline-3,14(4H)-dione (Compound 170)

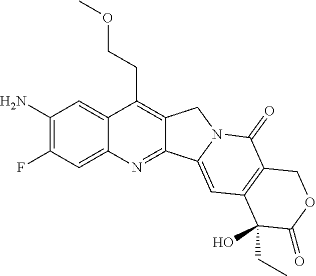 [SMILES: CC[C@@]1(O)C(=O)OCc2c1cc1n(c2=O)Cc2c-1nc1cc(F)c(N)cc1c2CCOC]

To a 10 mL round bottom flask containing Compound 3.4 (62.0 mg) was added water (0.89 mL), FeSO₄ (heptahydrate, 18.0 mg), and 3-methoxypropanal (113.0 mg). To the obtained suspension was added sulfuric acid (0.495 mL) dropwise while stirring at −15° C. in an ice salt bath. Hydrogen peroxide (0.118 mL) was then added dropwise. The mixture was stirred at −15° C. for 10 min and was then allowed to warm up to room temperature and stirred for 1 h. The reaction mixture was then diluted with water (30 mL) and the obtained suspension was extracted with DCM (3×30 mL). The organic phase was evaporated to dryness. Preparative HPLC purification was accomplished as described in General Procedure 9, eluting with a 25 to 45% CH₃CN/H₂O+0.1% TFA gradient to give the title compound as a dark orange solid (TFA salt, 3.1 mg, 4.4% yield).

LC/MS: Calc'd m/z=440.2 for C₂₃H₂₂FN₃O₅, found [M+H]⁺=440.2.

¹H NMR (300 MHz, DMSO-d6) δ 7.75 (d, J=12.4 Hz, 1H), 7.33 (d, J=9.4 Hz, 1H), 7.20 (s, 1H), 6.60-6.42 (m, 2H), 5.40 (s, 2H), 5.25 (s, 2H), 3.69 (t, J=6.5 Hz, 2H), 3.24 (s, 3H), 3.23 (t, J=6.5 Hz, 2H), 1.96-1.76 (m, 2H), 0.88 (t, J=7.3 Hz, 3H).

### 3.38: (S)—N-(4-ethyl-8-fluoro-4-hydroxy-3,14-dioxo-3,4,12,14-tetrahydro-1H-pyrano[3′,4′:6,7]indolizino[1,2-b]quinolin-9-yl)acetamide (Compound 171)

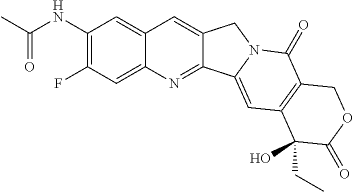 [SMILES: CC[C@@]1(O)C(=O)OCc2c1cc1n(c2=O)Cc2cc3cc(NC(C)=O)c(F)cc3nc2-1]

To a 25 mL round bottom flask containing acetic acid (0.071 mL) in dimethylformamide (0.69 mL) was added N-methylmorpholine (0.343 mL), HOAt (0.142 g), and HATU (0.435 g). After stirring at room temperature for 5 min, this solution was added to a 10 mL cone-shaped flask containing Compound 140 (0.127 g). This solution was stirred at room temperature for 24 h then directly purified by preparative HPLC as described in General Procedure 9, eluting with a 25 to 45% CH₃CN/H₂O+0.1% TFA gradient to give the title compound as a bright yellow powder (43.0 mg, 38% yield).

LC/MS: Calc'd m/z=424.1 for C₂₂H₁₈FN₃O₅, found [M+H]⁺=424.2.

¹H NMR (300 MHz, DMSO-d6) δ 10.13 (s, 1H), 8.73 (d, J=8.5 Hz, 1H), 8.61 (s, 1H), 7.96 (d, J=912.1 Hz, 1H), 7.29 (s, 1H), 6.60-6.42 (m, 2H), 5.41 (s, 2H), 5.21 (s, 2H), 2.20 (s, 3H), 1.96-1.76 (m, 2H), 0.88 (t, J=7.3 Hz, 3H).

### 3.39: tert-butyl (5-formyl-2-methoxy-4-nitrophenyl)carbamate (Compound 3.39)

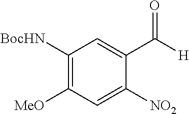 [SMILES: COc1cc([N+](=O)[O-])c(C=O)cc1NC(=O)OC(C)(C)C]

To a solution of Compound 3.2 (1.3 g, 1.0 eq.) in MeOH (12 mL) at 0° C. was added sodium methoxide (0.74 g, 3.0 eq.). After the addition was complete, the ice bath was removed and the resulting solution was stirred at room temperature for 72 h. The reaction was then quenched with ice water (50 mL) and extracted with DCM (3×100 mL). The combined organic layers were washed with brine (50 mL), dried over sodium sulfate, filtered, and concentrated in vacuo to yield the title compound as an orange solid (1.2 g, 89% yield).

LC/MS: Calc'd m/z=296.10 for C₁₃H₁₆N₂O₆, found [M+H]⁺=297.1.

¹H NMR (300 MHz, MeOD) δ 10.29 (s, 1H), 8.61 (s, 1H), 7.73 (s, 1H), 4.08 (s, 3H), 1.57 (s, 9H)

### 3.40: tert-butyl (4-amino-5-formyl-2-methoxyphenyl)carbamate (Compound 3.40)

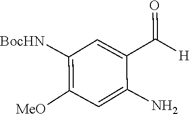 [SMILES: COc1cc(N)c(C=O)cc1NC(=O)OC(C)(C)C]

To a solution of Compound 3.39 (500 mg, 1 eq.) in MeOH (10 mL) and H₂O (1 mL) was added B₂(OH)₄ (454 mg, 3 eq.). The resulting mixture was cooled to 0° C. and an aqueous 5M NaOH solution (2.75 mL) was added with stirring over the course of 10 min. The reaction mixture was stirred for an additional 5 min then quenched by pouring the solution into ice (40 mL). The resulting mixture was extracted with DCM (3×50 mL), dried over sodium sulfate, filtered, and concentrated in vacuo. Flash purification was accomplished as described in General Procedure 9, using a 25 g silica column and eluting with 10 to 50% hexanes/EtOAc to give the title compound as an orange solid (386 mg, 86%).

LC/MS: Calc'd m/z=266.1 for C₁₃H₁₈N₂O₄, found [M+H]⁺=297.2.

### 3.41: (S)-9-amino-4-ethyl-8-fluoro-4-hydroxy-1,12-dihydro-14H-pyrano[3′,4′:6,7]indolizine o[1,2-b]quinoline-3,14(4H)-dione (Compound 168)

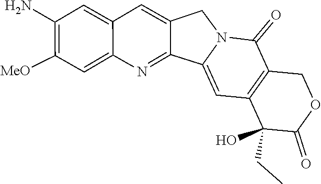 [SMILES: CC[C@@]1(O)C(=O)OCc2c1cc1n(c2=O)Cc2cc3cc(N)c(OC)cc3nc2-1]

A mixture of Compound 3.40 (385 mg, 1.0 eq.) and (S)-4-ethyl-4-hydroxy-7,8-dihydro-1H-pyrano[3,4-f]indolizine-3,6,10(4H)-trione (362 mg, 0.95 eq.), TsOH (monohydrate, 25 mg, 0.1 eq.) and toluene (30 mL) in a 250 mL round bottom flask equipped with a Dean-Stark apparatus was stirred at 110° C. for 2 h. The reaction mixture was then cooled to 25° C. and concentrated in vacuo. Purification was accomplished as described in General Procedure 9, using a 25 g silica column and eluting with a 0 to 50% DCM/MeOH gradient to provide the Boc-protected intermediate as a red solid. This material was then deprotecting according to General Procedure 6 followed by preparative HPLC purification as described in General Procedure 9, eluting with a 20 to 65% CH₃CN/H₂O+0.1% TFA gradient to give the title compound as a red solid (TFA salt, 300 mg, 53% yield).

LC/MS: Calc'd m/z=393.2 for C₂₁H₁₉N₃O₅, found [M+H]⁺=393.2.

¹H NMR (300 MHz, MeOD) δ 8.27 (s, 1H), 7.62 (s, 1H), 7.42 (s, 1H), 7.11 (s, 1H), 5.61 (d, J=16.2 Hz, 1H), 5.38 (d, J=16.2 Hz, 1H), 5.24 (s, 2H), 4.11 (s, 3H), 2.06-1.91 (m, 2H), 1.04 (t, J=7.4 Hz, 3H).

### 3.42: 5-bromo-2-nitro-4-(trifluoromethyl)benzaldehyde (Compound 3.42)

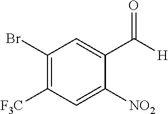 [SMILES: O=Cc1cc(Br)c(C(F)(F)F)cc1[N+](=O)[O-]]

To a stirring solution of HNO₃ (2.0 g, 1.4 mL, 67% purity, 2 eq.) in H₂SO₄ (8 mL) at 0° C. was added 3-bromo-4-(trifluoromethyl)benzaldehyde (4 g, 1 eq.). After the addition was complete, the ice bath was removed, and the reaction was allowed to stir for 5 h at room temperature. The mixture was poured into ice (100 mL) and the precipitate extracted with DCM (3×100 mL). The combined organic fractions were then washed with brine (50 mL), dried over Na₂SO₄, and concentrated in vacuo to yield the title compound as a yellow solid (4.4 g, 93% yield).

LC/MS: Calc'd m/z=296.90 for C₈H3BrF₃NO₃, found [M+H]⁺=298.0.

¹H NMR (300 MHz, MeOD) δ 10.35 (s, 1H), 8.29 (s, 1H), 8.23 (s, 1H).

### 3.43: tert-butyl (5-formyl-4-nitro-2-(trifluoromethyl)phenyl)carbamate (Compound 3.43)

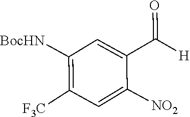 [SMILES: CC(C)(C)OC(=O)Nc1cc(C=O)c([N+](=O)[O-])cc1C(F)(F)F]

A mixture of Compound 3.42 (800 mg, 1 eq.), tert-butyl carbamate (378 mg, 1.2 eq.), Cs₂CO₃ (1.7 g, 2 eq.), Pd₂(dba)₃ (122 mg, 0.05 eq.), and dicyclohexyl[2′,4′,6′-tris(propan-2-yl)[1,1′-biphenyl]-2-yl]phosphane (XPhos) (256 mg, 0.2 eq.) in toluene (5 mL) was degassed and purged with N₂ for three cycles. The mixture was then stirred at 90° C. for 15 h under N₂ atmosphere. The reaction mixture was diluted with H₂O (25 mL) and extracted with EtOAc (3×50 mL). The combined organic layers were washed with brine (2×25 mL), dried over sodium sulfate, filtered, and concentrated under reduced pressure. Flash purification was achieved according to General Procedure 9, using a 25 g silica column and eluting with 0 to 25% DCM/MeOH to give the title compound as an orange solid (750 mg, 84% yield).

LC/MS: Calc'd m/z=334.1 for C₁₃H₁₃FN₂O₅, found [M−H]⁻=333.1.

### 3.44: tert-butyl (4-amino-5-formyl-2-(trifluoromethyl)phenyl)carbamate (Compound 3.44)

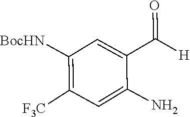 [SMILES: CC(C)(C)OC(=O)Nc1cc(C=O)c(N)cc1C(F)(F)F]

To a solution of Compound 3.43 (750 mg, 1 eq.) in MeOH (16 mL) and H₂O (1.6 mL) was added B₂(OH)₄ (603 mg, 3 eq.). The resulting mixture was cooled to 0° C. and an aqueous 5M NaOH solution (2.75 mL) was added with stirring over the course of 10 min. The reaction mixture was stirred for an additional 5 min then quenched by pouring the solution into ice (50 mL). The resulting mixture was extracted with DCM (3×75 mL), dried over sodium sulfate, filtered, and concentrated in vacuo. Flash purification was accomplished as described in General Procedure 9, using a 25 g silica column and eluting with 10 to 50% hexanes/EtOAc to give the title compound as an orange solid (460 mg, 67%).

LC/MS: Calc'd m/z=304.1 for C₁₃H₁₅F₃N₂O₃, found [M+H]⁺=305.2

### 3.45: (S)-9-amino-4-ethyl-4-hydroxy-8-(trifluoromethyl)-1,12-dihydro-14H-pyrano[3′,4′:6,7]indolizino [1,2-b]quinoline-3,14(4H)-dione (Compound 167)

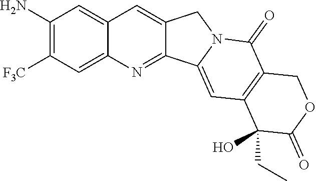 [SMILES: CC[C@@]1(O)C(=O)OCc2c1cc1n(c2=O)Cc2cc3cc(N)c(C(F)(F)F)cc3nc2-1]

A mixture of Compound 3.44 (460 mg, 1 eq.) and (S)-4-ethyl-4-hydroxy-7,8-dihydro-1H-pyrano[3,4-f]indolizine-3,6,10(4H)-trione (378 mg, 0.95 eq.), TsOH (monohydrate, 26 mg, 0.1 eq.) and toluene (35 mL) in a 250 mL round bottom flask equipped with a Dean-Stark apparatus was stirred at 110° C. for 2 h. The reaction mixture was then cooled to 25° C. and concentrated in vacuo. Purification was accomplished as described in General Procedure 9, using a 25 g silica column and eluting with a 0 to 50% DCM/MeOH gradient to provide the Boc-protected intermediate as a red solid. This material was then deprotecting according to General Procedure 6 followed by preparative HPLC purification as described in General Procedure 9, eluting with a 20 to 65% CH₃CN/H₂O+0.1% TFA gradient to give the titled compound as a yellow solid (6.2 mg, 48%).

LC/MS: Calc'd m/z=431.1 for C₂₁H₁₆F₃N₃O₄, found [M+H]⁺=432.2.

¹H NMR (300 MHz, MeOD) δ 8.29 (s, 1H), 8.27 (s, 1H), 7.59 (s, 1H), 7.24 (s, 1H), 5.59 (d, J=16.3 Hz, 1H), 5.39 (d, J=16.3 Hz, 1H), 5.28 (s, 2H), 2.00-1.89 (m, 2H), 1.03 (t, J=7.4 Hz, 3H).

### Example 4: Preparation of Drug-Linkers

### 4.1: 2,5-dioxopyrrolidin-1-yl (((9H-fluoren-9-yl)methoxy)carbonyl)glycylglycinate (Compound 4.1)

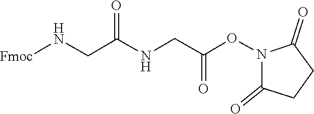

The title compound was prepared according to the procedure described in Chinese Patent Publication No. CN105218644.

### 4.2: (((9H-fluoren-9-yl)methoxy)carbonyl)glycylglycyl-L-phenylalanine (Fmoc-GGF-OH; Compound 4.2)

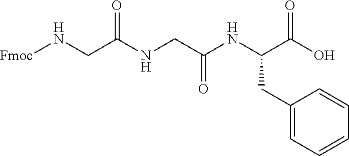

To L-phenylalanine (965 mg) in acetonitrile (10 mL) and dimethyl formamide (0.5 mL) was added DIPEA (1.51 mL) then Compound 4.1 (1.3 g). After 1 h the reaction was concentrated to dryness. Flash purification was accomplished as described in General Procedure 9, eluting with a 10 to 50% CH₃CN/H₂O+0.1% TFA gradient to provide the title compound as a white solid (430 mg, 30% yield).

LC/MS: Calc'd m/z=501.2 for C₂₈H₇₁N₃O₆S, found [M+H]⁺=502.4.

¹H NMR (300 MHz, DMSO) δ 8.16 (d, J=8.1 Hz, 1H), 8.04 (t, J=5.8 Hz, 1H), 7.90 (d, J=7.5 Hz, 2H), 7.72 (d, J=7.4 Hz, 2H), 7.59 (t, J=6.0 Hz, 1H), 7.54-7.39 (m, 2H), 7.33 (t, J=7.6 Hz, 2H), 7.28-7.13 (m, 5H), 4.44 (td, J=8.5, 5.1 Hz, 1H), 4.33-4.13 (m, 3H), 3.83-3.59 (m, 4H), 3.06 (dd, J=13.7, 5.1 Hz, 1H), 2.88 (dd, J=13.8, 9.0 Hz, 1H).

### 4.3: 2,3,5,6-tetrafluorophenyl 3-(2-(2-(2-(2,5-dioxo-2,5-dihydro-1H-pyrrol-1-yl)ethoxy) ethoxy)ethoxy)propanoate (MT-OTfp; Compound 4.3)

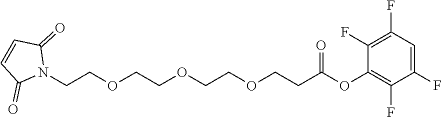 [SMILES: O=C(CCOCCOCCOCCN1C(=O)C=CC1=O)Oc1c(F)c(F)cc(F)c1F]

The title compound was prepared according to the procedure described in International Patent Publication No. WO 2017/054080.

### 4.4: (3-(2-(2-(2-(2,5-dioxo-2,5-dihydro-1H-pyrrol-1-yl)ethoxy)ethoxy)ethoxy)propanoyl) glycylglycyl-L-phenylalanine (Compound 4.4)

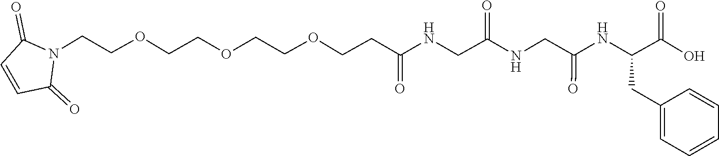 [SMILES: O=C(CCOCCOCCOCCN1C(=O)C=CC1=O)NCC(=O)NCC(=O)N[C@@H](Cc1ccccc1)C(=O)O]

To a solution of Compound 4.3 (1.61 g, 3.58 mmol) in DMF (35 mL) was added Gly-Gly-Phe (1 g, 3.58 mmol) as a single portion followed by iPr₂NEt (1.25 mL, 7.2 mmol). This solution was stirred at room temperature for 1 h, then evaporated to dryness. Purification was accomplished as described in General Procedure 9 using a 30 g C18 flash column and eluting with a 10 to 90% CH₃CN/H₂O+0.1% TFA gradient to provide the title compound as a white solid (400 mg, 20% yield).

LC/MS: Calc'd m/z=562.6 for C₂₆H₃₄N₄O₁₀, found [M−H]⁻=561.5.

¹H NMR (300 MHz, CDCl₃) δ 7.60 (t, J=5.6 Hz, 2H), 7.41 (d, J=7.7 Hz, 1H), 7.32-7.07 (m, 5H), 6.70 (s, 2H), 6.33-6.07 (m, 3H), 4.72 (td, J=7.6, 5.3 Hz, 1H), 4.12-3.78 (m, 4H), 3.72 (ddd, J=15.2, 6.9, 4.8 Hz, 5H), 3.60 (dd, J=11.6, 6.1 Hz, 10H), 3.12 (ddd, J=48.2, 14.0, 6.5 Hz, 2H), 2.52 (d, J=11.7 Hz, 2H).

### 4.5: (S)-11-benzyl-1-(9H-fluoren-9-yl)-3,6,9,12,15-pentaoxo-2-oxa-4,7,10,13,16-pentaazaheptadecan-17-yl acetate (Compound 4.5)

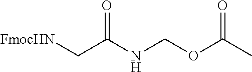

The title compound was prepared according to the procedure described in US Patent Publication No. US 2017/021031.

### 4.6: (S)-11-benzyl-1-(9H-fluoren-9-yl)-3,6,9,12,15-pentaoxo-2-oxa-4,7,10,13,16-pentaazaheptadecan-17-yl acetate (Compound 4.6)

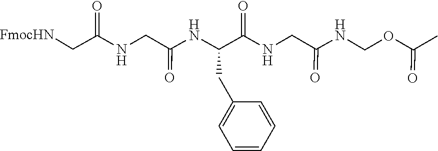

The title compound was prepared according to the procedure described in US Patent Publication No. US 2017/021031 using Fmoc-GGFGG-OH as the starting peptide.

### 4.7. tert-butyl (2-((2-(((S)-1-((2-((4-((4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)sulfonyl)phenyl)amino)-2-oxoethyl)amino)-1-oxo-3-phenylpropan-2-yl)amino)-2-oxoethyl)amino)-2-oxoethyl)carbamate (Compound 4.7)

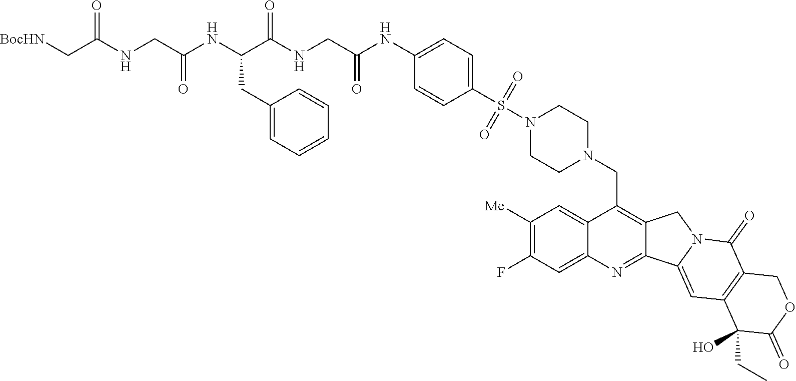 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)OC(C)(C)C)cc2)CC1]

The title compound was prepared according to General Procedure 7 starting from Compound 104 (20 mg). Preparative HPLC purification was accomplished as described in General Procedure 9, eluting with a 10 to 60% CH₃CN/H₂O+0.1% TFA gradient to provide the title compound as a white solid (14 mg, 42% yield).

LC/MS: Calc'd m/z=1051.4 for C₅₂H₅₈N₉O₁₂S, found [M+H]⁺=1052.6.

### 4.8: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-((4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)sulfonyl)phenyl)amino)-2-oxoethyl)-3-phenylpropanamide (MT-GGFG-Compound 104)

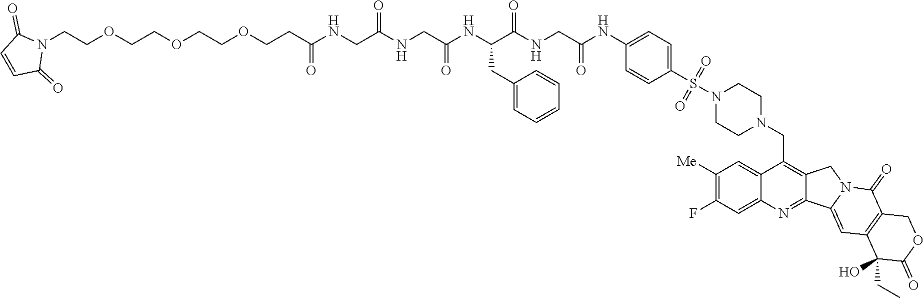 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)CCOCCOCCOCCN3C(=O)C=CC3=O)cc2)CC1]

The title compound was prepared according to Procedure 6 followed by Procedure 8 starting from Compound 4.7 (14 mg). Preparative HPLC purification was accomplished as described in General Procedure 9, eluting with a 10 to 60% CH₃CN/H₂O+0.1% TFA gradient to provide the title compound as a white solid (9.1 mg, 56% yield).

LC/MS: Calc'd m/z=1234.4 for C₆₀H₆₇FN₁₀O₁₆S, found [M+H]⁺=1235.8.

### 4.9: tert-butyl (2-((2-(((S)-1-((2-((4-(4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)phenyl)amino)-2-oxoethyl)amino)-1-oxo-3-phenylpropan-2-yl)amino)-2-oxoethyl)amino)-2-oxoethyl)carbamate (Compound 4.9)

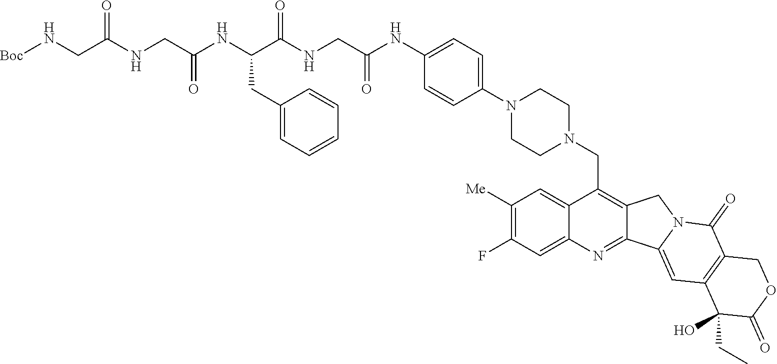 [SMILES: CC[C@@]1(O)C(=O)OCc2c1cc1n(c2=O)Cc2c-1nc1cc(F)c(C)cc1c2CN1CCN(c2ccc(NC(=O)CNC(=O)[C@H](Cc3ccccc3)NC(=O)CNC(=O)CNC(=O)OC(C)(C)C)cc2)CC1]

The title compound was prepared according to General Procedure 7 starting from Compound 108 (12 mg). Preparative HPLC purification was accomplished as described in General Procedure 9, eluting with a 10 to 60% CH₃CN/H₂O+0.1% TFA gradient to provide the title compound as a white solid (13 mg, 62% yield).

LC/MS: Calc'd m/z=987.4 for C₅₂H₅₈N₉O₁₀, found [M+H]⁺=988.6.

### 4.10: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-(4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)phenyl)amino)-2-oxoethyl)-3-phenylpropanamide (MT-GGFG-Compound 108)

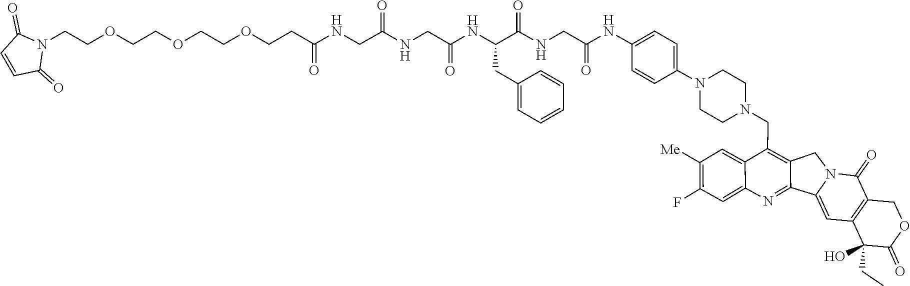 [SMILES: CC[C@@]1(O)C(=O)OCc2c1cc1n(c2=O)Cc2c-1nc1cc(F)c(C)cc1c2CN1CCN(c2ccc(NC(=O)CNC(=O)[C@H](Cc3ccccc3)NC(=O)CNC(=O)CNC(=O)CCOCCOCCOCCN3C(=O)C=CC3=O)cc2)CC1]

The title compound was prepared according to Procedure 6 followed by Procedure 8 starting from Compound 4.9 (13 mg). Preparative HPLC purification was accomplished as described in General Procedure 9, eluting with a 10 to 50% CH₃CN/H₂O+0.1% TFA gradient to provide the title compound as a white solid (3.1 mg, 20% yield).

LC/MS: Calc'd m/z=1170.5 for C₆₀H₆₇FN₁₀O₁₄, found [M+H]⁺=1171.6.

### 4.11: (9H-fluoren-9-yl)methyl (S)-(1-(4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,10-dioxo-7-oxa-2,4,9-triazaundecan-11-yl)carbamate (Compound 4.11)

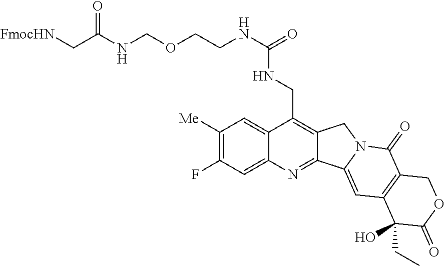

To a solution of Compound 1.2 (31 mg, 0.076 mmol) in DMF (750 μL) was added (9H-fluoren-9-yl)methyl (2-(((2-(((4-nitrophenoxy)carbonyl)amino)ethoxy)methyl)amino)-2-oxoethyl)carbamate (41 mg, 0.076 mmol) followed by iPr₂NEt (26 μL, 0.15 mmol). This solution was stirred at room temperature for 2 h and then applied directly to 12 g C18 column. Purification was accomplished as described in General Procedure 9, eluting with a 10 to 100% CH₃CN/H₂O+0.1% TFA gradient to provide the title compound as a white solid (21 mg, 35% yield).

LC/MS: Calc'd m/z=804.87 for C₄₃H₄₁FN₆O₉, found [M+H]⁺=805.6.

### 4.12: (S)-2-(1-(2,5-dioxo-2,5-dihydro-1H-pyrrol-1-yl)-12,15-dioxo-3,6,9-trioxa-13,16-diazaoctadecan-18-amido)-N-(1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,10-dioxo-7-oxa-2,4,9-triazaundecan-11-yl)-3-phenylpropanamide (MT-GGFG-AM-Compound 136)

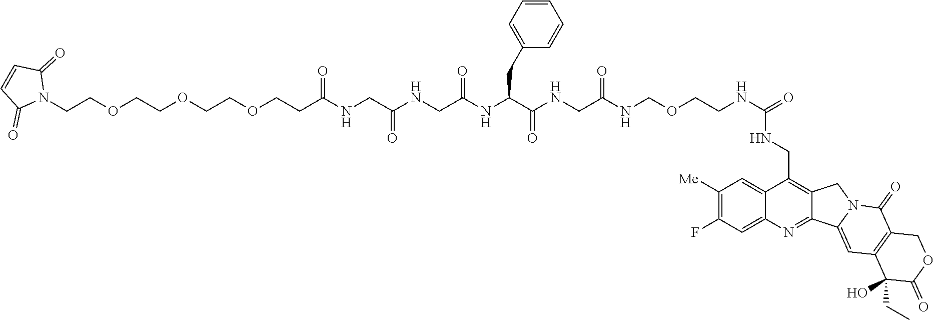 [SMILES: CC[C@@]1(O)C(=O)OCc2c1cc1n(c2=O)Cc2c-1nc1cc(F)c(C)cc1c2CNC(=O)NCCOCNC(=O)CNC(=O)[C@H](Cc1ccccc1)NC(=O)CNC(=O)CNC(=O)CCOCCOCCOCCN1C(=O)C=CC1=O]

Compound 4.11 (21 mg, 0.026 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL) followed by Compound 4.4 (15 mg, 0.026 mmol), NMM (10 μL) and HATU (10 mg, 0.026 mmol). Preparative HPLC purification was accomplished as described in General Procedure 9, eluting with a 30 to 60% CH₃CN/H₂O+0.1% TFA gradient to provide the title compound as a yellow solid (7.6 mg, 26% yield).

LC/MS: Calc'd m/z=1127.1 for C₅₄H₆₃FN₁₀O₁₆, found [M+H]⁺=1128.2.

### 4.13: (9H-fluoren-9-yl)methyl (2-(((2-(chlorosulfonyl)ethoxy)methyl)amino)-2-oxoethyl)carbamate (Compound 4.13)

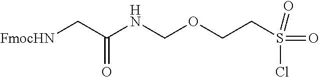

To a solution of Compound 4.5 (50 mg, 0.14 mmol), in DCM (800 μL) was added 2-hydroxyethane-1-sulfonyl chloride (100 mg, 0.7 mmol) followed by TFA (200 μL). This solution was stirred at room temperature for 30 min then evaporated to dryness. Purification was accomplished as described in General Procedure 9, using a 10 g silica column and eluting with a 10 to 100% EtOAc/Hexanes gradient to provide the title compound as a clear film (31 mg, 50% yield).

LC/MS: Calc'd m/z=452.1 for C₂₀H₂₁C₁N₂O₆S, found [M+Na]⁺=472.9

### 4.14: (9H-fluoren-9-yl)methyl (S)-(2-(((2-(N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl)ethoxy)methyl)amino)-2-oxoethyl)carbamate (Compound 4.14)

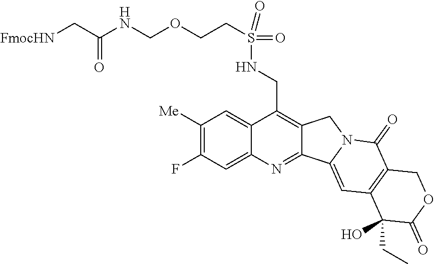

The title compound was prepared as described in General Procedure 3, using Compound 1.2 (28 mg, 0.07 mmol) and Compound 4.13 (31 mg, 0.07 mmol). Purification was accomplished as described in General Procedure 9, using a 12 g C18 column and eluting with a 10 to 100% CH₃CN/H₂O+0.1% TFA gradient to provide the title compound as a yellow solid (22 mg, 39% yield).

LC/MS: Calc'd m/z=825.9 for C₄₂H₄₀FN₅O₁₀S, found [M+H]⁺=826.7.

### 4.15: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((2-(N—(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl) ethoxy)methyl)amino)-2-oxoethyl)-3-phenylpropanamide (MT-GGFG-AM-Compound 129)

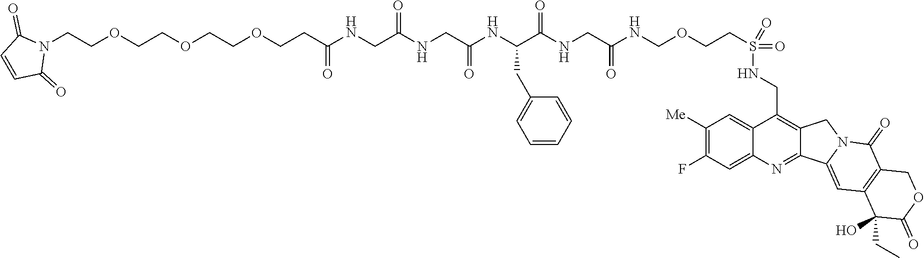 [SMILES: CC[C@@]1(O)C(=O)OCc2c1cc1n(c2=O)Cc2c-1nc1cc(F)c(C)cc1c2CNS(=O)(=O)CCOCNC(=O)CNC(=O)[C@H](Cc1ccccc1)NC(=O)CNC(=O)CNC(=O)CCOCCOCCOCCN1C(=O)C=CC1=O]

Compound 4.14 (22 mg, 0.027 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL) followed by Compound 4.4 (30 mg, 0.053 mmol), NMM (10 μL) and HATU (18 mg, 0.048 mmol). Preparative HPLC purification was accomplished as described in General Procedure 9, eluting with a 30 to 60% CH₃CN/H₂O+0.1% TFA gradient to provide the title compound as a yellow solid (6.4 mg, 21% yield).

LC/MS: Calc'd m/z=1148.2 for C₅₃H₆₂FN₉O₁₇S, found [M+H]⁺=1148.6.

¹H NMR (300 MHz, MeOD) δ 8.60 (t, J=6.5 Hz, 1H), 8.36 (t, J=8.6 Hz, 2H), 8.13 (d, J=6.6 Hz, 1H), 7.77 (d, J=10.6 Hz, 1H), 7.65 (d, J=4.8 Hz, 1H), 7.28-7.00 (m, 6H), 6.80 (s, 2H), 5.69-5.50 (m, 3H), 5.45-5.33 (m, 2H), 4.44 (dd, J=8.7, 5.7 Hz, 1H), 3.96 (t, J=5.3 Hz, 2H), 3.90-3.76 (m, 5H), 3.76-3.57 (m, 7H), 3.09-2.81 (m, 3H), 2.61-2.45 (m, 5H), 2.04-1.90 (m, 2H), 1.03 (t, J=7.3 Hz, 3H).

### 4.16: (9H-fluoren-9-yl)methyl (S)-(2-(((morpholin-2-ylmethoxy)methyl)amino)-2-oxoethyl)carbamate (Compound 4.16)

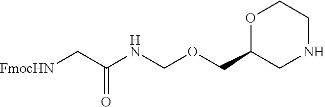

To a solution of Compound 4.5 (100 mg, 0.27 mmol) in DCM (800 μL) was added (S)-morpholin-2-ylmethanol (160 mg, 1.36 mmol) followed by TFA (200 μL). This solution was stirred at room temperature for 1 h then evaporated to dryness. Purification was accomplished as described in General Procedure 9, using a 12 g C18 flash column and eluting with a 10 to 90% CH₃CN/H₂O+0.1% TFA gradient to provide the title compound as a yellow solid (TFA salt, 105 mg, 72% yield).

LC/MS: Calc'd m/z=425.2 for C₂₃H₂₇N₃O₅, found [M+Na]⁺=448.0.

### 4.17. (9H-fluoren-9-yl)methyl (2-(((((S)-4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)morpholin-2-yl)methoxy)methyl)amino)-2-oxoethyl)carbamate (Compound 4.17)

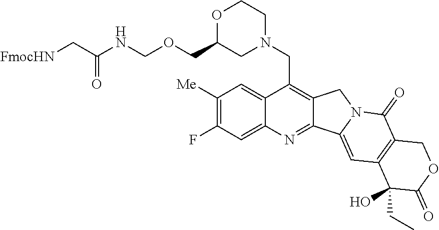

The title compound was prepared according to General Procedure 1 starting from Compound 1.1 (50 mg, 0.117 mmol) and Compound 4.16 (63 mg, 0.117 mmol). Preparative HPLC purification was accomplished as described in General Procedure 9, eluting with a 10 to 100% CH₃CN/H₂O+0.1% TFA gradient to give the title compound as an off-white solid (TFA salt, 33 mg, 35% yield).

LC/MS: Calc'd m/z=817.9 for C₄₅H₄₄FN₅O₉, found [M+H]⁺=818.7.

### 4.18: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((((S)-4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)morpholin-2-yl)methoxy)methyl)amino)-2-oxoethyl)-3-phenylpropanamide (MT-GGFG-AM-Compound 113)

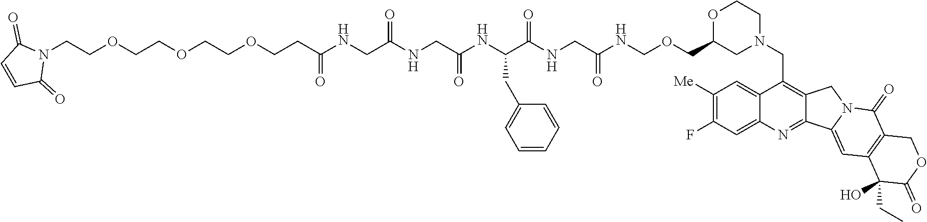 [SMILES: CC[C@@]1(O)C(=O)OCc2c1cc1n(c2=O)Cc2c-1nc1cc(F)c(C)cc1c2CN1CCO[C@H](COCNC(=O)CNC(=O)[C@H](Cc2ccccc2)NC(=O)CNC(=O)CNC(=O)CCOCCOCCOCCN2C(=O)C=CC2=O)C1]

Compound 4.17 (33 mg, 0.04 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (100 μL) and DCM (900 μL) followed by Compound 4.4 (45 mg, 0.08 mmol), NMM (20 μL) and HATU (28 mg, 0.073 mmol). Preparative HPLC purification was accomplished as described in General Procedure 9, eluting with a 30 to 60% CH₃CN/H₂O+0.1% TFA gradient to provide the title compound as a yellow solid (22 mg, 48% yield).

LC/MS: Calc'd m/z=1140.2 for C₅₆H₆₆FN₉O₁₆, found [M+H]⁺=1141.1.

¹H NMR (300 MHz, MeOD) δ 8.35 (d, J=7.5 Hz, 2H), 7.74-7.61 (m, 1H), 7.53 (s, 1H), 7.34-7.10 (m, 6H), 6.81 (s, 2H), 5.65-5.30 (m, 4H), 4.64 (t, J=3.4 Hz, 2H), 4.42 (tt, J=6.3, 2.5 Hz, 1H), 4.09 (d, J=12.3 Hz, 1H), 3.98-3.76 (m, 8H), 3.72 (t, J=6.0 Hz, 2H), 3.69-3.44 (m, 17H), 3.21-2.85 (m, 3H), 2.64-2.42 (m, 5H), 2.03-1.84 (m, 2H), 0.98 (t, J=7.3 Hz, 3H).

### 4.19: (9H-fluoren-9-yl)methyl (S)-(2-((((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methoxy)methyl) amino)-2-oxoethyl)carbamate (Compound 4.19)

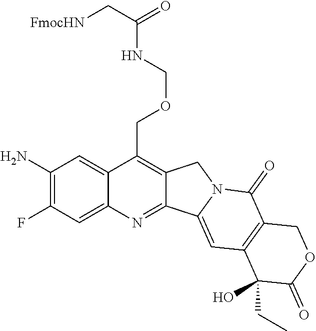

Compound 3.5 (55 mg, 0.11 mmol) was dissolved in TFA (500 μL) and stirred at room temperature for 20 min, then hexafluoroisopropanol (2 mL) was added followed by Compound 4.5 (40 mg, 0.11 mmol). This solution was stirred at room temperature for ~16 h then concentrated to dryness. Purification was accomplished as described in General Procedure 9, using a 12 g C18 flash column and eluting with a 10 to 50% CH₃CN/H₂O+0.1% TFA gradient to provide the title compound as a yellow solid (11 mg, 14% yield).

LC/MS: Calc'd m/z=719.7 for C₃₉H₃₄FN₅O₈, found [M+H]⁺=720.6.

### 4.20: (S)—N-(2-(((((S)-9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methoxy)methyl)amino)-2-oxoethyl)-2-(1-(2,5-dioxo-2,5-dihydro-1H-pyrrol-1-yl)-12,15-dioxo-3,6,9-trioxa-13,16-diazaoctadecan-18-amido)-3-phenylpropanamide (MT-GGFG-AM-Compound 141)

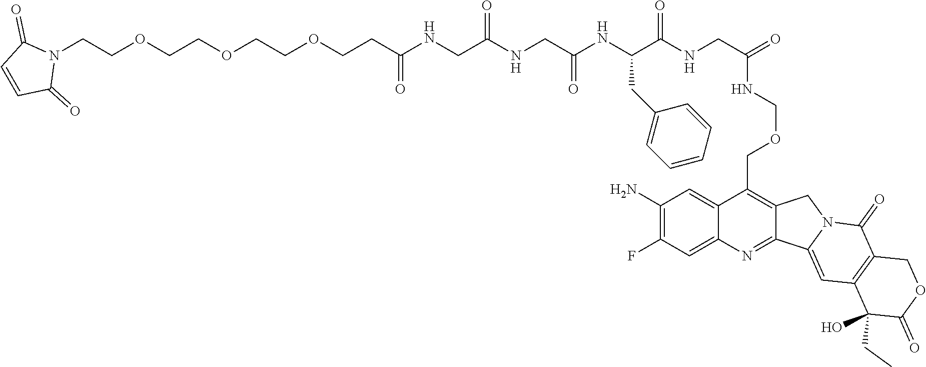 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCOCCOCCOCCN1C(=O)C=CC1=O]

Compound 4.19 (11 mg, 0.015 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL) followed by Compound 4.4 (26 mg, 0.045 mmol), NMM (5 μL) and HATU (18 mg, 0.045 mmol). Preparative HPLC purification was accomplished as described in General Procedure 9, eluting with a 32 to 45% CH₃CN/H₂O+0.1% TFA gradient to provide the title compound as a yellow solid (4.6 mg, 29% yield).

LC/MS: Calc'd m/z=1142.0 for C₅₀H₅₆FN₉O₁₅, found [M+H]⁺=1143.1.

¹H NMR (300 MHz, MeOD) δ 8.36 (s, 1H), 8.28 (d, J=6.1 Hz, 1H), 8.16 (dd, J=20.1, 6.8 Hz, 3H), 7.59-7.44 (m, 2H), 7.31-7.08 (m, 6H), 6.79 (s, 2H), 5.58 (d, J=16.1 Hz, 1H), 5.37 (d, J=16.1 Hz, 1H), 5.30-5.16 (m, 3H), 4.56-4.39 (m, 1H), 4.07-3.90 (m, 2H), 3.85 (dt, J=11.5, 5.4 Hz, 4H), 3.79-3.67 (m, 4H), 3.67-3.55 (m, 7H), 3.54 (d, J=6.5 Hz, 8H), 3.10 (dd, J=14.0, 6.1 Hz, 1H), 2.92 (dd, J=13.9, 9.1 Hz, 1H), 2.53 (t, J=6.0 Hz, 2H), 1.98 (q, J=7.2 Hz, 2H), 1.31 (s, 1H), 1.04 (t, J=7.3 Hz, 3H).

### 4.21: N—((S)-1-((S)-9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)-9-benzyl-5,8,11,14-tetraoxo-2-oxa-4,7,10,13-tetraazapentadecan-15-yl)-6-(2,5-dioxo-2,5-dihydro-1H-pyrrol-1-yl)hexanamide (MC-GGFG-AM-Compound 141)

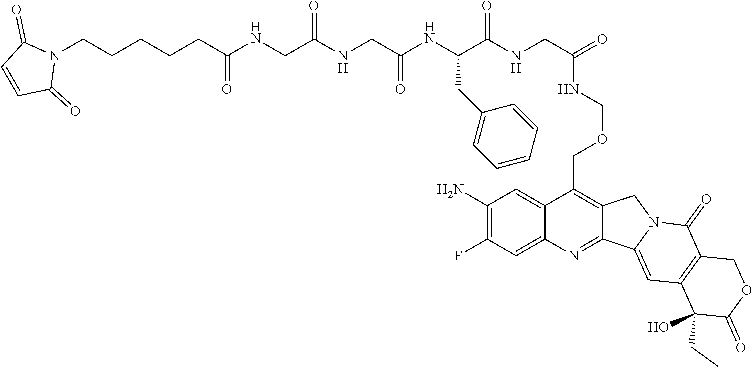 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCCCCN1C(=O)C=CC1=O]

Compound 4.19 (25 mg, 0.035 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL), followed by MC-GGF-OH (33 mg, 0.07 mmol), NMM (20 μL) and HATU (25 mg, 0.066 mmol). Preparative HPLC purification was accomplished as described in General Procedure 9, eluting with a 30 to 50% CH₃CN/H₂O+0.1% TFA gradient to provide the title compound as a yellow solid (4.3 mg, 13% yield).

LC/MS: Calc'd m/z=952.0 for C₄₇H₅₀FN₉O₁₂, found [M+H]⁺=952.9.

¹H NMR (300 MHz, CD₃CN) δ 7.96-7.72 (m, 1H), 7.39-7.07 (m, 8H), 6.94 (d, J=9.1 Hz, 1H), 6.73 (s, 2H), 5.44 (d, J=16.2 Hz, 1H), 5.25 (d, J=16.2 Hz, 1H), 5.06 (d, J=4.4 Hz, 2H), 4.81 (d, J=26.1 Hz, 4H), 4.61 (s, 1H), 3.96 (s, 1H), 3.77 (d, J=8.1 Hz, 7H), 3.02 (d, J=5.6 Hz, 5H), 2.19 (t, J=7.7 Hz, 3H), 1.50 (dp, J=14.8, 7.4 Hz, 6H), 1.32-1.12 (m, 3H), 0.96 (t, J=7.2 Hz, 3H).

### 4.22: tert-butyl (2-((2-(((S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)carbamate (Compound 4.22)

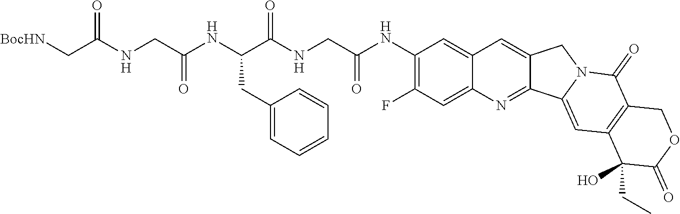 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)OC(C)(C)C)c(F)cc3nc2-1]

The title compound was prepared according to Procedure 7 starting from Compound 140 (28 mg). Preparative HPLC purification was accomplished as described in General Procedure 9, eluting with a 10 to 50% CH₃CN/H₂O+0.1% TFA gradient to provide the title compound as a white solid (10 mg, 17% yield).

LC/MS: Calc'd m/z=799.3 for C₄₀H₄₂N₇O₁₀, found [M+H]⁺=800.6.

### 4.23: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 140)

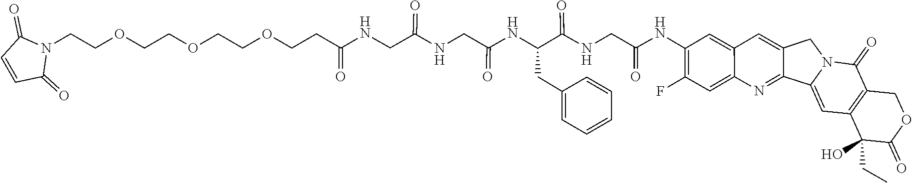 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCOCCOCCOCCN4C(=O)C=CC4=O)c(F)cc3nc2-1]

The title compound was prepared according to General Procedure 6 followed by General Procedure 8 starting from Compound 4.22 (10 mg). Preparative HPLC purification was accomplished as described in General Procedure 9, eluting with a 10 to 50% CH₃CN/H₂O+0.1% TFA gradient to provide the title compound as a white solid (6.8 mg, 55% yield).

LC/MS: Calc'd m/z=982.4 for C₄₈H₅₁FN₈O₁₄, found [M+H]⁺=983.6.

### 4.24: tert-butyl (2-((2-(((S)-1-((2-(((S)-4-ethyl-8-fluoro-4-hydroxy-11-(morpholinomethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)carbamate (Compound 4.24)

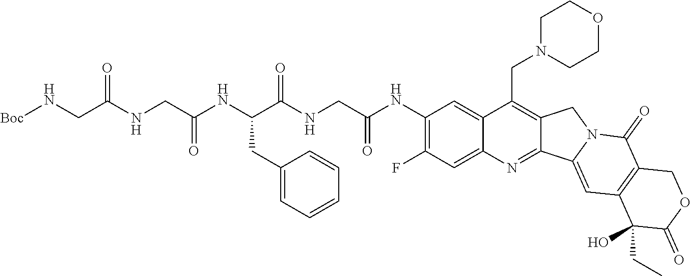 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)OC(C)(C)C)cc1c2CN1CCOCC1]

The title compound was prepared according to General Procedure 7 starting from Compound 142 (TFA salt, 45 mg). Purification was accomplished as described in General Procedure 9, using a 12 g C18 flash column and eluting with a 10 to 50% CH₃CN/H₂O+0.1% TFA gradient to provide the title compound as a white solid (13 mg, 22% yield).

LC/MS: Calc'd m/z=898.4 for C₄₅H₅₁N₈O₁₁, found [M+H]⁺=899.6.

### 4.25: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-11-(morpholinomethyl)-3,14-dioxo-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 142)

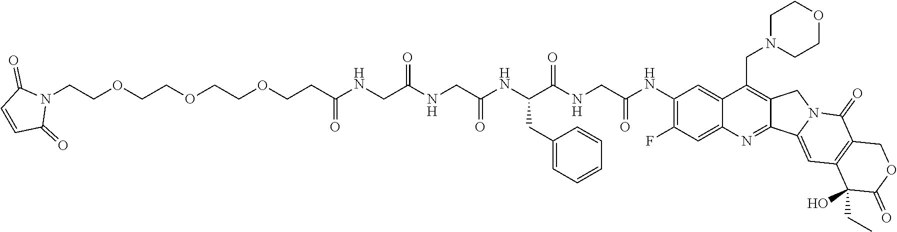 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCOCC1]

The title compound was prepared according to General Procedure 6 followed by General Procedure 8 starting from Compound 4.24 (13 mg). Preparative HPLC purification was accomplished as described in General Procedure 9, eluting with a 10 to 50% CH₃CN/H₂O+0.1% TFA gradient to provide the title compound as a white solid (2.6 mg, 17% yield).

LC/MS: Calc'd m/z=1081.4 for C₅₃H₆₀FN₉O₁₅, found [M+H]⁺=1082.6.

¹H NMR (300 MHz, MeOD) δ 9.34 (d, J=8.5 Hz, 1H), 7.87 (d, J=11.8 Hz, 1H), 7.62 (s, 1H), 7.33-7.19 (m, 5H), 6.80 (s, 2H), 5.62 (d, J=16.3 Hz, 1H), 5.51 (s, 2H), 5.47-5.35 (m, 3H), 4.73 (dd, J=9.6, 5.1 Hz, 1H), 4.61 (s, 3H), 4.30-4.15 (m, 2H), 4.11 (s, 2H), 4.00-3.82 (m, 4H), 3.82-3.70 (m, 7H), 3.70-3.50 (m, 13H), 3.18-3.04 (m, 1H), 2.88 (s, 1H), 2.64 (d, J=5.8 Hz, 4H), 2.54 (t, J=6.0 Hz, 2H), 2.09-1.92 (m, 2H), 1.03 (t, J=7.3 Hz, 3H).

### 4.26: (9H-fluoren-9-yl)methyl (S)-(12-benzyl-1-(4-nitrophenoxy)-1,8,11,14,17-pentaoxo-2,5-dioxa-7,10,13,16-tetraazaoctadecan-18-yl)carbamate (Compound 4.26)

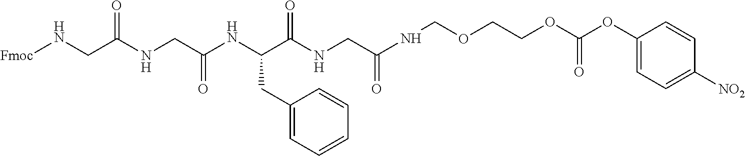

To a stirring solution of Compound 4.6 (60 mg) in dichloromethane (2 mL) was added ethylene glycol (100 μL) followed by trifluoracetic acid (0.4 mL). After 30 min the reaction was concentrated in vacuo. Purification of the intermediate compound was accomplished as described in General Procedure 9, using a 10 g flash column and eluting with a 0 to 20% dichloromethane/methanol gradient. To the purified intermediate in tetrahydrofuran (0.5 mL) was added bis-nitrophenol carbonate (58 mg) followed by DIPEA (50 μL). The solution was stirred for 16 h, quenched with acetic acid (~100 μL) then concentrated to dryness. Purification was accomplished as described in General Procedure 9, using a 10 g flash column and eluting with a 0 to 20% dichloromethane/methanol gradient to provide the title compound as a white solid (40 mg, 53% yield from Compound 4.6).

LC/MS: Calc'd m/z=796.3 for C₄₀H₄₀N₆O₁₂, found [M+Na]⁺=819.4.

### 4.27: (S)-16-amino-10-benzyl-6,9,12,15-tetraoxo-3-oxa-5,8,11,14-tetraazahexadecyl (((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 4.27)

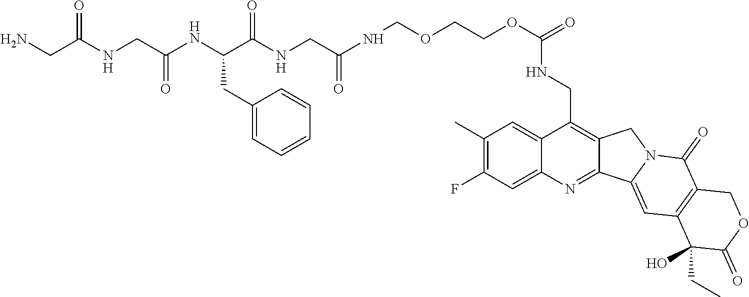 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CN]

To a solution of Compound 4.26 (40 mg) in dimethylformamide (1 mL) was added DIPEA (26 μL) then a solution of Compound 1.2 (24 mg) in dimethylformamide (0.5 mL). This solution was stirred for 4 h at room temperature then quenched with a 20% piperidine in dimethylformamide solution (0.5 mL) and stirred for an additional 20 min. Purification was accomplished as described in General Procedure 9, using a 12 g C18 flash column and eluting with a 10 to 50% CH₃CN/H₂O+0.1% TFA gradient to provide the title compound as a white solid (TFA salt, 19 mg, 39% yield).

LC/MS: Calc'd m/z=844.3 for C₄₁H₄₅FN₈O₁₁, found [M+H]⁺=845.6.

### 4.28: (S)-10-benzyl-29-(2,5-dioxo-2,5-dihydro-1H-pyrrol-1-yl)-6,9,12,15,18-pentaoxo-3,21,24,27-tetraoxa-5,8,11,14,17-pentaazanonacosyl (((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (MT-GGFG-AM-Compound 139)

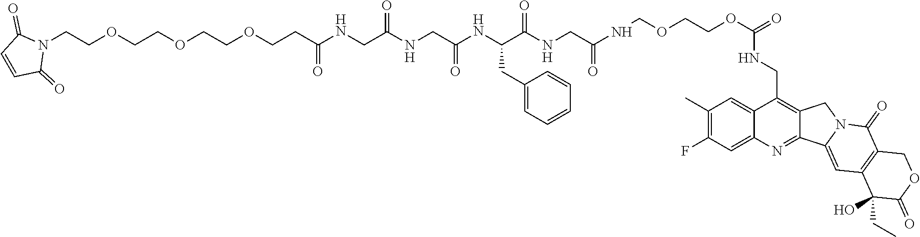 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCOCCOCCOCCN1C(=O)C=CC1=O]

The title compound was prepared according to General Procedure 8 starting from Compound 4.27 (10 mg). Preparative HPLC purification was accomplished as described in General Procedure 9, eluting with a 10 to 50% CH₃CN/H₂O+0.1% TFA gradient to provide the title compound as a white solid (8.8 mg, 75% yield).

LC/MS: Calc'd m/z=1127.4 for C₅₄H₆₂FN₉O₁₇, found [M+H]⁺=1128.6.

¹H NMR (300 MHz, MeOD) δ 8.27 (d, J=8.1 Hz, 1H), 7.81 (d, J=10.7 Hz, 1H), 7.65 (s, 1H), 7.32-7.16 (m, 5H), 6.81 (s, 2H), 5.62 (d, J=16.4 Hz, 1H), 5.53 (s, 2H), 5.42 (d, J=16.4 Hz, 1H), 4.93 (s, 2H), 4.67 (s, 1H), 4.51 (dd, J=9.3, 5.6 Hz, 1H), 4.18 (t, J=4.7 Hz, 2H), 4.01-3.44 (m, 19H), 3.17 (dd, J=13.9, 5.8 Hz, 1H), 2.97 (dd, J=13.9, 9.0 Hz, 1H), 2.57 (s, 3H), 2.52 (t, J=6.0 Hz, 2H), 2.03-1.91 (m, 2H), 1.03 (t, J=7.4 Hz, 3H).

### 4.29: (S)-2-amino-N-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)acetamide (Compound 4.29)

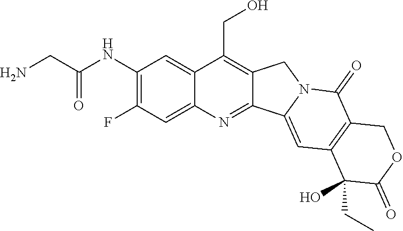 [SMILES: CC[C@@]1(O)C(=O)OCc2c1cc1n(c2=O)Cc2c-1nc1cc(F)c(NC(=O)CN)cc1c2CO]

To a stirring solution of Fmoc-glycine (217 mg) in dimethylformamide (2.5 mL) was added HATU (254 mg), HOAt (83 mg) then NMM (188 μL). This solution was stirred for 10 min then Compound 141 (50 mg) was added and the reaction was stirred at room temperature for 16 h. Lithium hydroxide (2.5 mL, 1 M in water) was added, and the reaction mixture was stirred for 2 h. This solution was partially concentrated, then a solution of 20% piperidine in dimethylformamide (0.5 mL) was added and was stirred for another 20 min. The reaction was then evaporated onto celite and purification was accomplished as described in General Procedure 9, using a 12 g C18 flash column and eluting with a 0 to 40% CH₃CN/H₂O+0.1% TFA gradient to provide the title compound as a white solid (TFA salt, 44 mg, 62% yield).

LC/MS: Calc'd m/z=468.1 for C₂₃H₂₁FN₄O₆, found [M+H]⁺=469.4.

¹H NMR (300 MHz, MeOD) δ 8.99 (d, J=8.3 Hz, 1H), 7.99 (s, 1H), 7.87 (d, J=12.0 Hz, 1H), 7.55 (s, 1H), 5.60 (d, J=16.3 Hz, 1H), 5.46-5.35 (m, 3H), 5.30 (s, 2H), 3.53-3.45 (m, 1H), 3.43-3.38 (m, 1H), 2.03-1.87 (m, 2H), 1.02 (t, J=7.3 Hz, 3H).

### 4.30: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 141)

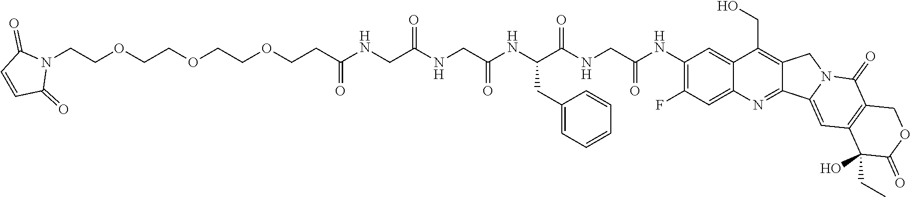 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CO]

To a stirring solution of Compound 4.4 (23 mg) in a mixture of dimethylformamide (0.1 mL) and dichloromethane (0.9 mL) was added HATU (14 mg), a solution of Compound 4.29 (20 mg) in dimethyl formamide (0.1 mL) and dichloromethane (0.9 mL), and DIPEA (24 μL). The mixture was stirred for 15 min, then the reaction was partially concentrated. Preparative HPLC purification was accomplished as described in General Procedure 9, eluting with a 0 to 40% CH₃CN/H₂O+0.1% TFA gradient to provide the title compound as a white solid (7.1 mg, 20% yield).

LC/MS: Calc'd m/z=1012.4 for C₄₉H₅₃FN₈O₁₅, found [M+H]⁺=1013.6.

¹H NMR (300 MHz, MeOD) δ 9.89 (s, 1H), 8.75 (d, J=8.3 Hz, 1H), 8.44-8.32 (m, 1H), 8.27-8.14 (m, 2H), 7.78 (d, J=11.9 Hz, 1H), 7.53 (s, 1H), 7.39-7.20 (m, 5H), 6.82 (s, 2H), 5.57 (d, J=16.3 Hz, 1H), 5.39 (d, J=16.3 Hz, 1H), 5.34-5.25 (m, 2H), 5.22 (s, 2H), 4.32-4.09 (m, 2H), 3.96-3.83 (m, 3H), 3.76 (t, J=6.0 Hz, 2H), 3.69-3.62 (m, 2H), 3.62-3.47 (m, 9H), 3.40-3.33 (m, 1H), 3.08 (dd, J=14.0, 9.6 Hz, 1H), 2.56 (t, J=6.1 Hz, 2H), 2.03-1.91 (m, 2H), 1.04 (t, J=7.3 Hz, 3H).

### 4.31: tert-butyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 4.31)

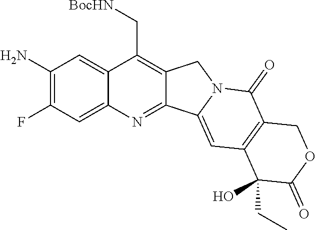 [SMILES: CC[C@@]1(O)C(=O)OCc2c1cc1n(c2=O)Cc2c-1nc1cc(F)c(N)cc1c2CNC(=O)OC(C)(C)C]

To a stirring solution of Compound 145 (32 mg) in dichloromethane (2 mL) and acetonitrile (0.5 mL) was added di-tert-butyl dicarbonate (20 μL) followed by DIPEA (42 μL). The reaction mixture was stirred at room temperature for 3 h then concentrated to dryness to provide the title compound as a red solid (34 mg, 87%).

LC/MS: Calc'd m/z=510.2 for C₂₆H₂₇FN₄O₆, found [M+H]⁺=511.2.

### 4.32: tert-butyl (S)-((9-(2-aminoacetamido)-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 4.32)

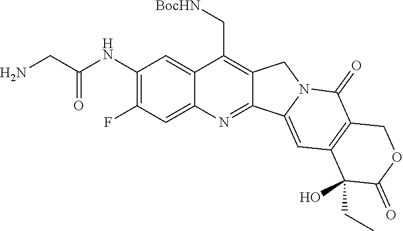 [SMILES: CC[C@@]1(O)C(=O)OCc2c1cc1n(c2=O)Cc2c-1nc1cc(F)c(NC(=O)CN)cc1c2CNC(=O)OC(C)(C)C]

To a stirring solution of Fmoc-glycine (98 mg) in dimethylformamide (1 mL) was added HATU (115 mg), HOAt (37 mg) then NMM (85 μL). This solution was stirred for 10 min, then Compound 4.31 (28 mg) was added. The reaction was stirred at room temperature for 16 h then quenched with a solution of 20% piperidine in dimethylformamide (1 mL) and stirred for an additional 20 min. Purification was accomplished as described in General Procedure 9, using a 12 g C18 flash column and eluting with a 5 to 40% CH₃CN/H₂O+0.1% TFA gradient to provide the title compound as a white solid (TFA salt, 25 mg, 67% yield).

LC/MS: Calc'd m/z=567.2 for C₂₈H₃₀FN₆O₇, found [M+H]⁺=568.4.

¹H NMR (300 MHz, MeOD) δ 9.01 (d, J=8.3 Hz, 1H), 7.83 (d, J=11.9 Hz, 1H), 7.52 (s, 1H), 5.57 (d, J=16.4 Hz, 1H), 5.38 (d, J=16.3 Hz, 1H), 5.27 (d, J=3.1 Hz, 2H), 4.80 (s, 2H), 4.10 (s, 2H), 1.97 (q, J=7.4 Hz, 2H), 1.50 (s, 9H), 1.02 (t, J=7.3 Hz, 3H).

### 4.33: tert-butyl (((S)-9-(2-((S)-2-(2-(2-aminoacetamido)acetamido)-3-phenylpropanamido)acetamido)-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 4.33)

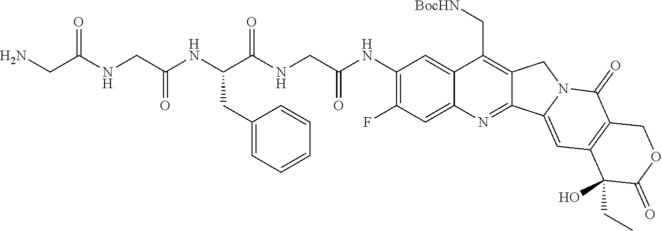 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CN)cc1c2CNC(=O)OC(C)(C)C]

To a stirring solution of Fmoc-GGF-OH (28 mg) and HATU (20 mg) in a mixture of DMF (0.2 mL) and dichloromethane (1.8 mL) was added Compound 4.32 (25 mg) followed by DIPEA (32 μL). This solution was stirred for 15 min at room temperature, quenched with a solution of 20% piperidine in dimethylformamide (0.250 mL), stirred for an additional 20 min, then partially concentrated in vacuo. Purification was accomplished as described in General Procedure 9, using a 12 g C18 flash column and eluting with a 10 to 45% CH₃CN/H₂O+0.1% TFA gradient to provide the title compound as a white solid (TFA salt, 22 mg, 64% yield).

LC/MS: Calc'd m/z=828.3 for C₄₁H₄₅FN₈O₁₀, found [M+H]⁺=829.6.

### 4.34: (S)—N-(2-(((S)-11-(aminomethyl)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)-2-(1-(2,5-dioxo-2,5-dihydro-1H-pyrrol-1-yl)-12,15-dioxo-3,6,9-trioxa-13,16-diazaoctadecan-18-amido)-3-phenylpropanamide (MT-GGFG-Compound 145)

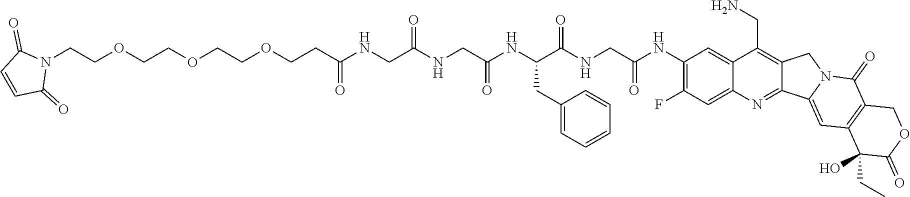 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN]

The title compound was prepared according to Procedure 6 followed by Procedure 8 starting from Compound 4.33 (15 mg). Preparative HPLC purification of the intermediate Boc-protected compound was accomplished as described in General Procedure 9, eluting with a 10 to 50% CH₃CN/H₂O+0.1% TFA gradient. The title compound was obtained post Boc-deprotection as a white-solid (TFA salt, 8.5 mg, 52% yield).

LC/MS: Calc'd m/z=1011.4 for C₄₉H₅₃FN₈O₁₅, found [M+H]⁺=1012.6.

¹H NMR (300 MHz, MeOD) δ 9.04 (d, J=8.0 Hz, 1H), 8.40 (d, J=5.7 Hz, 1H), 8.21 (d, J=7.7 Hz, 1H), 8.05 (d, J=11.5 Hz, 1H), 7.67 (s, 1H), 7.42-7.03 (m, 5H), 6.81 (s, 2H), 5.63 (d, J=16.4 Hz, 1H), 5.51 (s, 1H), 5.43 (d, J=16.5 Hz, 1H), 4.81 (s, 2H), 4.75-4.58 (m, 1H), 4.29-4.10 (m, 2H), 3.98-3.81 (m, 4H), 3.78-3.71 (m, 2H), 3.71-3.63 (m, 2H), 3.62-3.53 (m, 9H), 3.14-2.98 (m, 1H), 2.54 (t, J=6.0 Hz, 2H), 2.08-1.93 (m, 2H), 1.03 (t, J=7.3 Hz, 3H).

### 4.355: (9H-fluoren-9-yl)methyl(S)-(2-((4-ethyl-8-fluoro-4-hydroxy-3,14-dioxo-11-(piperidin-1-ylmethyl)-3,4,12,14-tetrahydro-1H-pyrano[3′,4′:6,7]indolizino[1,2-b]quinolin-9-yl)amino)-2-oxoethyl)carbamate (Compound 4.35)

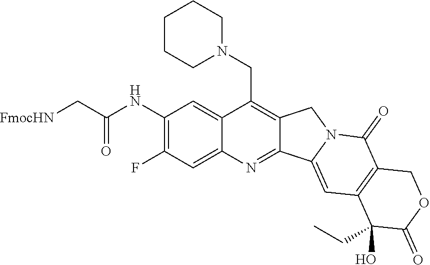

To a solution of Fmoc-Gly-OH (100.9 mg, 0.34 mmol) in dimethylformamide (550 μL) was added NMM (0.112 mL, 1.02 mmol) and HATU (0.103 g, 0.272 mmol). This solution was stirred at room temperature for 20 min, then a solution of Compound 148 (32.5 mg, 0.068 mmol) in DMF (250 μL) was added, and the reaction mixture was stirred for 16 h. Purification was accomplished as described in General Procedure 9, using a 12 g C18 flash column and eluting with a 5 to 40% CH₃CN/H₂O+0.1% TFA gradient. The obtained residue was re-purified according to General Procedure 9, using a 10 g flash column and eluting with a 0 to 10% MeOH/DCM gradient to provide the title compound as a yellow powder (15.3 mg, 30% yield).

LC/MS: Calc'd m/z=757.3 for C₄₃H₄₀FN₅O₇, found [M+H]⁺=758.6.

### 4.36: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-3,14-dioxo-11-(piperidin-1-ylmethyl)-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 148)

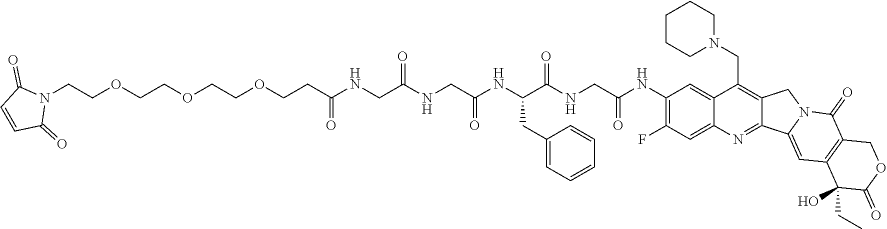 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCCCC1]

To a 50 mL flask containing Compound 4.35 (15.3 mg, 0.02 mmol) was added a solution of 20% piperidine in DMF (2.0 mL). This solution was stirred at room temperature for 5 min then evaporated to dryness. The obtained residue was then dissolved in 10% DMF/DCM (1.0 mL), then NMM (5.50 μL, 0.05 mmol), Compound 4.4 (11.2 mg, 0.02 mmol) and HATU (8.7 mg, 0.02 mmol) were added. This solution was stirred for 45 min, then partially evaporated. Preparative HPLC purification was accomplished as described in General Procedure 9, eluting with a 15 to 45% CH₃CN/H₂O+0.1% TFA gradient to give the title product as a yellow powder (7.8 mg, 33% yield).

LC/MS: Calc'd m/z=1079.4 for C₅₄H₆₂FN₉O₁₄, found [M+H]⁺=1080.8.

¹H NMR (300 MHz, MeOD) δ 8.99 (d, J=8.2 Hz, 1H), 7.52 (d, J=12.3 Hz, 1H), 7.39-7.25 (m, 5H), 7.25-7.17 (m, 1H), 6.79 (s, 2H), 5.53 (d, J=16.4 Hz, 1H), 5.33 (d, J=16.5 Hz, 1H), 4.80-4.72 (m, 1H), 4.32-4.11 (m, 2H), 3.98-3.79 (m, 6H), 3.76 (t, J=6.0 Hz, 2H), 3.66-3.60 (m, 2H), 3.62-3.49 (m, 10H), 3.15-3.03 (m, 1H), 2.65-2.47 (m, 6H), 1.96 (q, J=7.4 Hz, 2H), 1.72-1.57 (m, 4H), 1.57-1.42 (m, 2H), 1.03 (t, J=7.4 Hz, 3H).

### 4.37. tert-butyl (2-((2-(((S)-1-((2-((4-(N—(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl)phenyl)amino)-2-oxoethyl)amino)-1-oxo-3-phenylpropan-2-yl)amino)-2-oxoethyl)amino)-2-oxoethyl)carbamate (Compound 4.37)

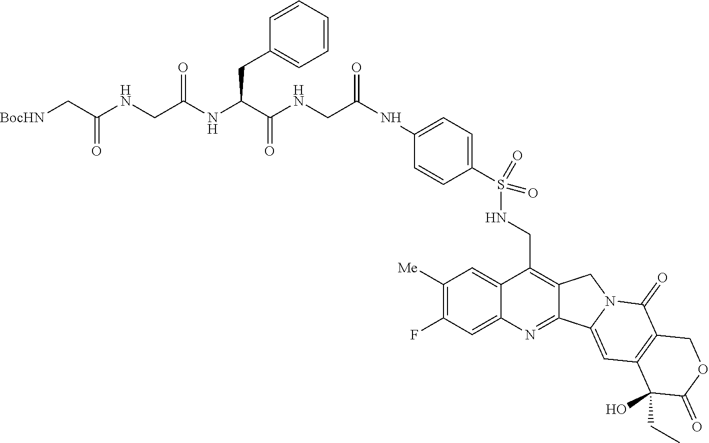 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)OC(C)(C)C)cc1]

The title compound was prepared according to General Procedure 7 starting from Compound 127 (46 mg). Preparative HPLC purification was accomplished as described in General Procedure 9, eluting with a 10 to 60% CH₃CN/H₂O+0.1% TFA gradient to provide the title compound as a white solid (7.2 mg, 21% yield).

LC/MS: Calc'd m/z=983.0 for C₄₈H₅₁N₈O₁₂S, found [M+H]⁺=983.9.

### 4.38: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-(N—(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl) phenyl)amino)-2-oxoethyl)-3-phenylpropanamide (MT-GGFG-Compound 127)

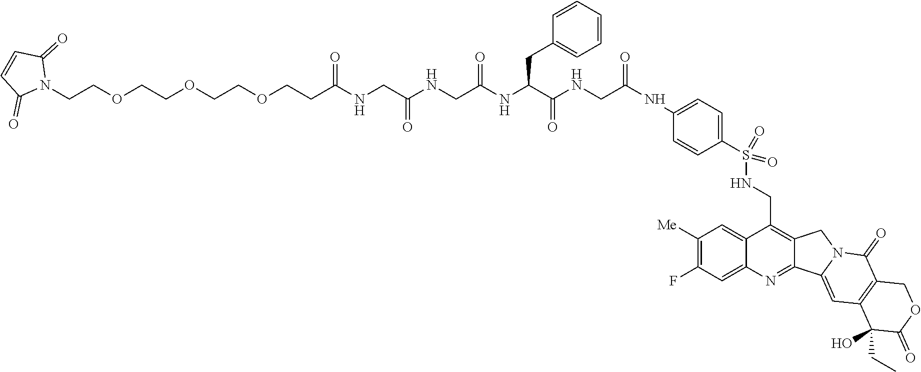 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)CCOCCOCCOCCN2C(=O)C=CC2=O)cc1]

The title compound was prepared according to Procedure 6 followed by Procedure 8 starting from Compound 4.37 (7.2 mg). Preparative HPLC purification was accomplished as described in General Procedure 9, eluting with a 10 to 50% CH₃CN/H₂O+0.1% TFA gradient to provide the title compound as a white solid (1 mg, 12% yield).

LC/MS: Calc'd m/z=1166.2 for C₅₆H₆₀FN₉O₁₆, found [M+H]⁺=1167.1

### 4.39: (9H-fluoren-9-yl)methyl ((7S)-1-((3-azabicyclo[3.1.1]heptan-6-yl)oxy)-7-benzyl-3,6,9,12-tetraoxo-2,5,8,11-tetraazatridecan-13-yl)carbamate (Compound 4.39)

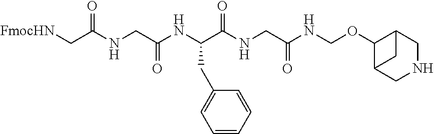

To a stirring solution of Compound 4.6 (44 mg) in dichloromethane (2 mL) was added 3-azabicyclo[3.1.1]heptan-6-ol (5.3 mg) followed by trifluoracetic acid (0.4 mL). After 30 min the reaction was concentrated in vacuo. Purification was accomplished as described in General Procedure 9, using a 10 g flash column and eluting with a 0 to 20% dichloromethane/methanol gradient to provide the title compound as a white solid (14.7 mg, 46% yield).

LC/MS: Calc'd m/z=682.8 for C₃₇H₄₂N₆O₇, found [M+H]⁺=683.6.

### 4.40: (2S)-2-(2-(2-aminoacetamido)acetamido)-N-(2-((((3-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-azabicyclo[3.1.1]heptan-6-yl)oxy)methyl)amino)-2-oxoethyl)-3-phenylpropanamide (Compound 4.40)

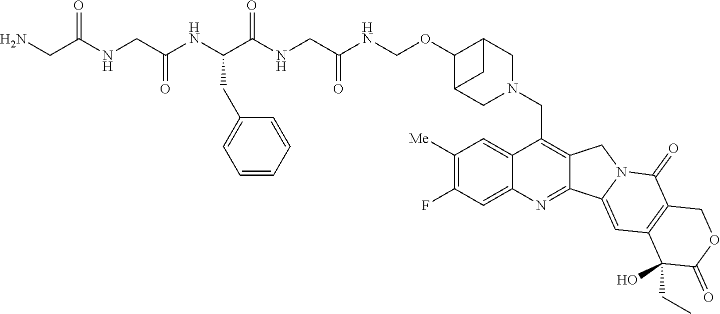 [SMILES: CC[C@@]1(O)C(=O)OCc2c1cc1n(c2=O)Cc2c-1nc1cc(F)c(C)cc1c2CN1CC2CC(C1)C2OCNC(=O)CNC(=O)[C@H](Cc1ccccc1)NC(=O)CNC(=O)CN]

The title compound was prepared according to General Procedure 1 starting from Compound 1.1 (3 mg, 0.007 mmol) and Compound 4.39 (14.7 mg, 0.022 mmol) and utilizing 200 μL DMF. Following complete consumption of Compound 1.1, a solution of 20% piperidine in DMF (200 μL) was added and this solution was stirred at room temperature for 10 min. Preparative HPLC purification was accomplished as described in General Procedure 9, eluting with a 20 to 37% CH₃CN/H₂O+0.1% TFA gradient to give the title compound as an off-white solid (TFA salt, 1.8 mg, 29% yield).

LC/MS: Calc'd m/z=852.9 for C₄₄H₄₉FN₈O₉, found [M+H]⁺=853.7.

### 4.41: (2S)-2-(1-(2,5-dioxo-2,5-dihydro-1H-pyrrol-1-yl)-12,15-dioxo-3,6,9-trioxa-13,16-diazaoctadecan-18-amido)-N-(2-((((3-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-azabicyclo[3.1.1]heptan-6-yl)oxy)methyl)amino)-2-oxoethyl)-3-phenylpropanamide (MT-GGFG-AM-Compound 117)

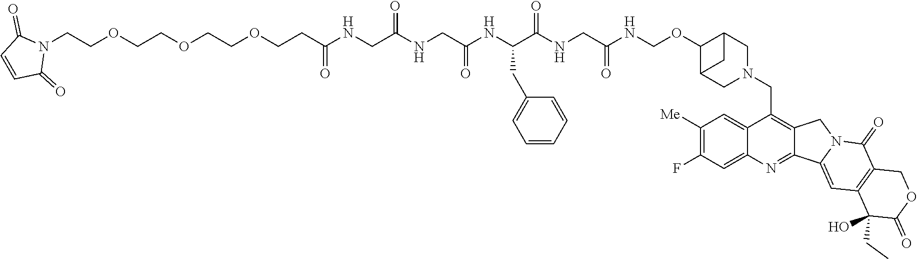 [SMILES: CC[C@@]1(O)C(=O)OCc2c1cc1n(c2=O)Cc2c-1nc1cc(F)c(C)cc1c2CN1CC2CC(C1)C2OCNC(=O)CNC(=O)[C@H](Cc1ccccc1)NC(=O)CNC(=O)CNC(=O)CCOCCOCCOCCN1C(=O)C=CC1=O]

The title compound was prepared according to Procedure 8 starting from Compound 4.40 (1.8 mg). Preparative HPLC purification was accomplished as described in General Procedure 9, eluting with a 25 to 45% CH₃CN/H₂O+0.1% TFA gradient to provide the title compound as a white-solid (TFA salt, 0.5 mg, 22% yield).

LC/MS: Calc'd m/z=1135.5 for C₅₇H₆₆FN₉O₁₅, found [M+H]⁺=1136.3.

### 4.42: (9H-fluoren-9-yl)methyl (S)-(9-benzyl-1-(3-fluoroazetidin-3-yl)-5,8,11,14-tetraoxo-2-oxa-4,7,10,13-tetraazapentadecan-15-yl)carbamate (Compound 4.42)

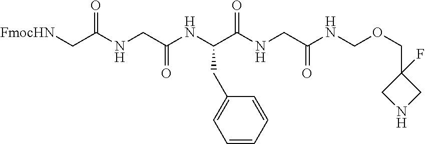

To a stirring solution of Compound 4.6 (144 mg) in dichloromethane (2 mL) was added (3-fluoroazetidin-3-yl)methanol (16 mg) followed by trifluoracetic acid (0.4 mL). After 30 min the reaction was concentrated in vacuo. Purification was accomplished as described in General Procedure 9, using a 10 g flash column and eluting with a 0 to 20% dichloromethane/methanol gradient to provide the title compound as a white solid (55 mg, 54% yield).

LC/MS: Calc'd m/z=674.7 for C₃₅H₃₉N₆FO₇, found [M+H]⁺=675.6.

### 4.43: (S)-2-(2-(2-aminoacetamido)acetamido)-N-(2-((((1-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-fluoroazetidin-3-yl)methoxy)methyl)amino)-2-oxoethyl)-3-phenylpropanamide (Compound 4.43)

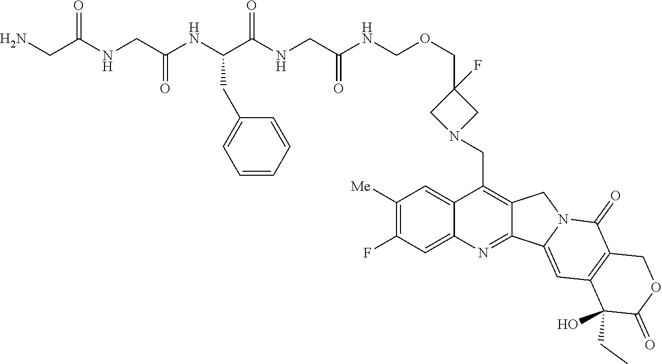 [SMILES: CC[C@@]1(O)C(=O)OCc2c1cc1n(c2=O)Cc2c-1nc1cc(F)c(C)cc1c2CN1CC(F)(COCNC(=O)CNC(=O)[C@H](Cc2ccccc2)NC(=O)CNC(=O)CN)C1]

The title compound was prepared according to General Procedure 1 starting from Compound 1.1 (11.6 mg, 0.027 mmol) and Compound 4.42 (55 mg, 0.082 mmol) and utilizing 500 μL DMF. Following complete consumption of Compound 1.1, a solution of 20% piperidine in DMF (500 μL) was added and this solution was stirred at room temperature for 10 min. Preparative HPLC purification was accomplished as described in General Procedure 9, eluting with a 25 to 32% CH₃CN/H₂O+0.1% TFA gradient to give the title compound as an off-white solid (TFA salt, 8.1 mg, 28% yield).

LC/MS: Calc'd m/z=844.3 for C₄₂H₄₆F₂N₈O₉, found [M+H]⁺=845.3

### 4.44: (S)-2-(1-(2,5-dioxo-2,5-dihydro-1H-pyrrol-1-yl)-12,15-dioxo-3,6,9-trioxa-13,16-diazaoctadecan-18-amido)-N-(2-((((1-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-fluoroazetidin-3-yl)methoxy)methyl)amino)-2-oxoethyl)-3-phenylpropanamide (MT-GGFG-AM-Compound 118)

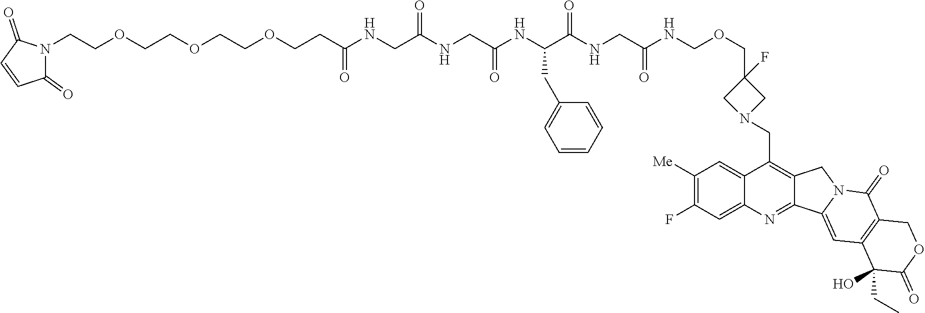 [SMILES: CC[C@@]1(O)C(=O)OCc2c1cc1n(c2=O)Cc2c-1nc1cc(F)c(C)cc1c2CN1CC(F)(COCNC(=O)CNC(=O)[C@H](Cc2ccccc2)NC(=O)CNC(=O)CNC(=O)CCOCCOCCOCCN2C(=O)C=CC2=O)C1]

The title compound was prepared according to Procedure 8 starting from Compound 4.43 (8.1 mg). Preparative HPLC purification was accomplished as described in General Procedure 9, eluting with a 25 to 45% CH₃CN/H₂O+0.1% TFA gradient to provide the title compound as a white-solid (TFA salt, 2.9 mg, 28% yield).

LC/MS: Calc'd m/z=1127.4 for C₅₅H₆₃F₂N₉O₁₅, found [M+H]⁺=1128.8.

### 4.45: (S)-10-benzyl-23-(2,5-dioxo-2,5-dihydro-1H-pyrrol-1-yl)-6,9,12,15,18-pentaoxo-3-oxa-5,8,11,14,17-pentaazatricosyl (((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (MC-GGFG-AM-Compound 139)

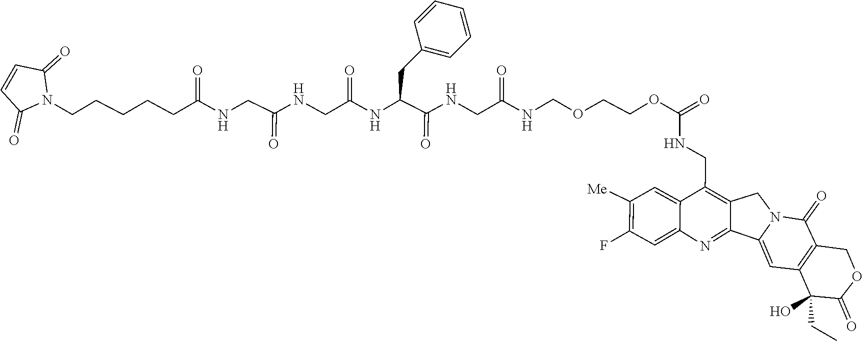 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCCCCN1C(=O)C=CC1=O]

To Compound 4.27 (450 mg) was added a solution of 2,5-dioxopyrrolidin-1-yl 6-(2,5-dioxopyrrol-1-yl)hexanoate (130 mg) and N-ethyldiisopropylamine (250 μL) in DMF (10 mL). This solution was stirred at room temperature for 30 min then concentrated to −1 mL volume. Purification was accomplished as described in General Procedure 9 first using a 60 g C18 flash column and eluting with a 10 to 60% CH₃CN/H₂O+0.1% TFA gradient followed by preparative HPLC of impure fractions using a 20 to 50% CH₃CN/H₂O+0.1% TFA gradient to provide the title compound as a white solid (320 mg, 66% yield).

LC/MS: Calc'd m/z=1037.4 for C₅₁H₅₆FN₉O₁₄, found [M+H]⁺=1038.6.

¹H NMR (300 MHz, MeOD) δ 8.10 (d, J=8.1 Hz, 2H), 8.01 (s, 1H), 7.95 (d, J=7.0 Hz, 1H), 7.74 (d, J=10.4 Hz, 1H), 7.66 (s, 1H), 7.56 (s, 1H), 7.32-7.10 (m, 5H), 6.69 (s, 2H), 5.63 (d, J=16.4 Hz, 1H), 5.46 (s, 2H), 5.32 (s, 1H), 5.28 (d, J=16.5 Hz, 1H), 4.88 (s, 2H), 4.67 (d, J=6.4 Hz, 2H), 4.48 (d, J=7.1 Hz, 2H), 4.15 (t, J=4.2 Hz, 2H), 3.92 (dd, J=17.1, 6.2 Hz, 2H), 3.83-3.57 (m, 6H), 3.46 (t, J=7.1 Hz, 2H), 3.16 (dd, J=14.0, 5.9 Hz, 1H), 2.95 (dd, J=13.9, 8.9 Hz, 1H), 2.53 (s, 3H), 2.21 (t, J=7.6 Hz, 2H), 1.97-1.79 (m, 2H), 1.58 (dp, J=15.0, 7.6 Hz, 4H), 1.29 (dd, J=16.6, 9.3 Hz, 3H), 1.01 (t, J=7.3 Hz, 3H).

### 4.46: tert-butyl (S)-(2-((4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)carbamate (Compound 4.46)

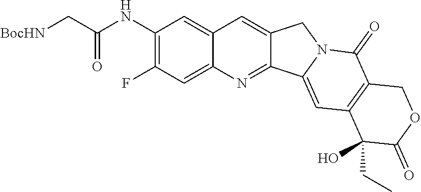 [SMILES: CC[C@@]1(O)C(=O)OCc2c1cc1n(c2=O)Cc2cc3cc(NC(=O)CNC(=O)OC(C)(C)C)c(F)cc3nc2-1]

A solution of Compound 140 (860 mg, 1.7 mmol, TFA salt), Boc-Gly-OH (760 mg, 4.3 mmol), HATU (1.6 g, 4.1 mmol), and N-ethyldiisopropylamine (0.6 mL) in DMF (4 mL) was stirred at room temperature for 24 h then poured into water (50 mL). The resulting solid was collected by filtration, redissolved in 10% MeOH/DCM and purification was accomplished as described in General Procedure 9, using a 30 g silica column and eluting with a 0 to 10% MeOH/DCM to provide the title compound as a yellow solid (750 mg, 80% yield).

LC/MS: Calc'd m/z=538.5 for C₂₇H₂₇FN₄O₇, found [M+H]⁺=539.4.

¹H NMR (300 MHz, MeOD) δ 8.84 (d, J=8.4 Hz, 1H), 8.52 (s, 1H), 8.00 (s, 1H), 7.87 (d, J=12.1 Hz, 1H), 7.62 (s, 1H), 5.60 (d, J=16.3 Hz, 1H), 5.40 (d, J=16.4 Hz, 1H), 5.27 (s, 2H), 4.02 (s, 2H), 1.99 (dt, J=8.7, 6.7 Hz, 2H), 1.52 (s, 9H), 1.03 (t, J=7.4 Hz, 3H).

### 4.47: (S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-aminium (Compound 4.47)

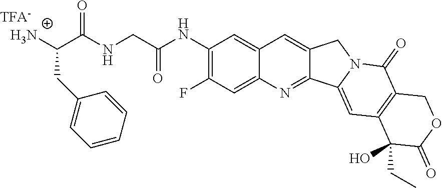 [SMILES: CC[C@@]1(O)C(=O)OCc2c1cc1n(c2=O)Cc2cc3cc(NC(=O)CNC(=O)[C@@H]([NH3+])Cc4ccccc4)c(F)cc3nc2-1]

The title compound was prepared in three steps from Compound 4.46 (750 mg). The Boc protecting group was cleaved in neat TFA (2 mL) followed by precipitation in Et₂O (50 mL). The solid was collected by filtration and added to a solution of 2,5-dioxopyrrolidin-1-yl (2S)-2-[(tert-butoxycarbonyl)amino]-3-phenylpropanoate (340 mg, 1.1 equiv) and N-ethyldiisopropylamine (300 μL) in DMF (1.7 mL). This solution was stirred at room temperature for 30 min then pipetted into Et₂O (50 mL). The precipitate was collected by filtration, dried under vacuum then dissolved in neat TFA (2 mL). After 20 min, Et₂O (50 mL) was added and the precipitate collected by filtration to provide the title compound as a yellow solid (531 mg, 54% yield).

LC/MS: Calc'd m/z=585.2 for C₃₁H₂₈FN₅O₆, found [M+H]⁺=586.1.

### 4.48: 2-((2-(((S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethan-1-aminium (Compound 4.48)

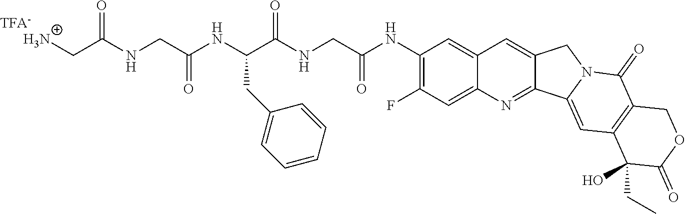 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)C[NH3+])c(F)cc3nc2-1]

To Compound 4.47 (490 mg) was added a solution of Boc-gly-gly-NHS (250 mg, 1.1 equiv) and N-ethyldiisopropylamine (250 μL) in DMF (3 mL). This solution was stirred at room temperature for 30 min then pipetted into Et₂O (50 mL). The precipitate was collected by filtration then dissolved in neat TFA (2 mL). After 20 min, Et₂O (50 mL) was added and the precipitate collected by filtration to provide the title compound as a yellow solid (500 mg, 88% yield).

LC/MS: Calc'd m/z=699.2 for C₃₅H₃₄FN₇O₈, found [M+H]⁺=700.4.

### 4.49: 6-(2,5-dioxo-2,5-dihydro-1H-pyrrol-1-yl)-N-(2-((2-(((S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)hexanamide (MC-GGFG-Compound 140)

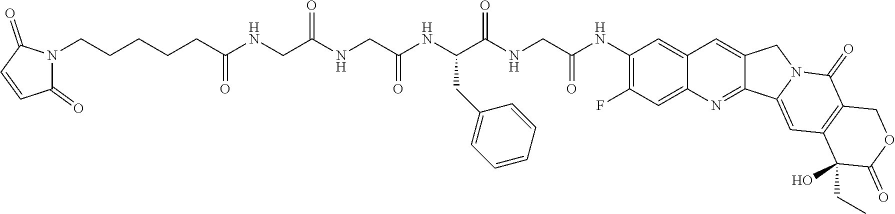 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCCCN4C(=O)C=CC4=O)c(F)cc3nc2-1]

To Compound 4.48 (500 mg) was added a solution of 2,5-dioxocyclopentyl 6-(2,5-dioxopyrrol-1-yl)hexanoate (210 mg, 1.1 equiv) and N-ethyldiisopropylamine (215 μL) in DMF (4 mL). This solution was stirred at room temperature for 30 min then pipetted into Et₂O (50 mL). The precipitate was collected by filtration then dissolved in DMF (2 mL). Preparative HPLC purification was accomplished as described in General Procedure 9, eluting with a 24 to 38% CH₃CN/H₂O+0.1% TFA gradient to provide the title compound as a yellow solid (190 mg, 40% yield).

LC/MS: Calc'd m/z=892.9 for C₄₅H₄₅FN₈O₁₁, found [M+H]⁺=893.6.

¹H NMR (300 MHz, CD₃CN) δ 8.67 (d, J=8.4 Hz, 1H), 8.44 (s, 1H), 7.78 (d, J=12.1 Hz, 1H), 7.41 (s, 1H), 7.30 (d, J=4.3 Hz, 4H), 7.26-7.16 (m, 1H), 6.72 (s, 2H), 5.52 (d, J=16.4 Hz, 1H), 5.31 (d, J=16.4 Hz, 1H), 5.12 (s, 2H), 4.64 (dd, J=9.7, 5.0 Hz, 1H), 4.11 (d, J=3.2 Hz, 2H), 3.87-3.68 (m, 4H), 3.37 (t, J=7.1 Hz, 2H), 3.00 (dd, J=14.0, 9.7 Hz, 1H), 2.20 (t, J=7.6 Hz, 2H), 1.49 (dq, J=19.5, 7.4 Hz, 4H), 1.22 (p, J=7.6, 7.1 Hz, 2H), 0.94 (t, J=7.3 Hz, 3H).

### 4.50: tert-butyl (S)-(2-((4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamate (Compound 4.50)

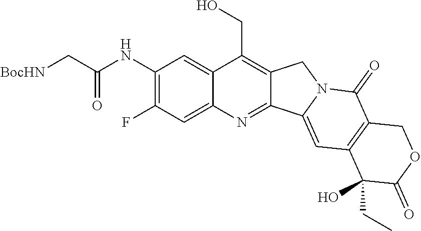 [SMILES: CC[C@@]1(O)C(=O)OCc2c1cc1n(c2=O)Cc2c-1nc1cc(F)c(NC(=O)CNC(=O)OC(C)(C)C)cc1c2CO]

A solution of Compound 4.46 (1.8 g), iron (II) sulfate heptahydrate (1.4 g, 1.5 equiv), and sulfuric acid (450 μL, 2.5 equiv) in MeOH (33 mL) was heated to 60° C. and hydrogen peroxide (1.25 mL, 12 equiv) was added dropwise over 10 min. This solution was heated for another 20 min then cooled to room temperature and poured into ice water (~200 mL). The brown precipitate was collected by filtration and the filtrate was quenched with saturated aqueous Na₂S₂O₃. MeOH was evaporated and the solution allowed to stand for 2 h while a second brown precipitate formed. This precipitate was collected by filtration and the combined precipitates were purified as described in General Procedure 9 using a 50 g silica column and eluting with a 0 to 15% MeOH/DCM gradient to provide the title compound as a yellow solid (860 mg, 45% yield).

LC/MS: Calc'd m/z=568.5 for C₂₈H₂₉FN₄O₈, found [M+H]⁺=569.7.

### 4.51: (S)-1-((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-aminium (Compound 4.51)

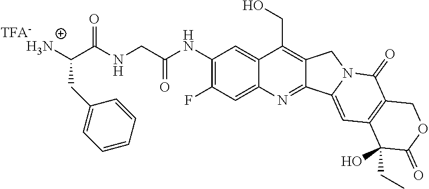

The title compound was prepared in three steps from Compound 4.50 (750 mg). The Boc protecting group was cleaved in neat TFA (2 mL) followed by precipitation in Et₂O (100 mL). The solid was collected by filtration and added to a solution of 2,5-dioxopyrrolidin-1-yl (2S)-2-[(tert-butoxycarbonyl)amino]-3-phenylpropanoate (600 mg, 1.1 equiv) and N-ethyldiisopropylamine (300 μL) in DMF (7 mL). This solution was stirred at room temperature for 30 min then pipetted into Et₂O (100 mL). The precipitate was collected by filtration, dried under vacuum then dissolved in neat TFA (2 mL). After 20 min, Et₂O (100 mL) was added and the precipitate collected by filtration to provide the title compound as a yellow solid (756 mg, 78% yield).

LC/MS: Calc'd m/z=615.2 for C₃₂H₃₀FN₅O₇, found [M+H]⁺=616.3.

### 4.52: 2-((2-(((S)-1-((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethan-1-aminium (Compound 4.52)

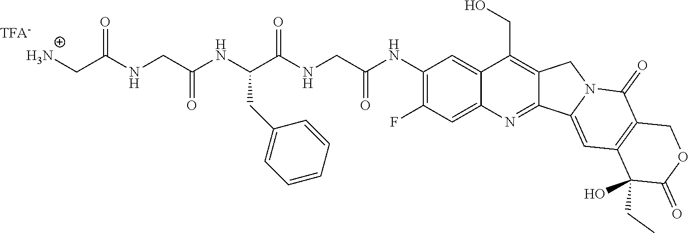 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)C[NH3+])cc1c2CO]

To Compound 4.51 (756 mg) was added a solution of Boc-gly-gly-NHS (375 mg, 1.1 equiv) and N-ethyldiisopropylamine (400 μL) in DMF (5 mL). This solution was stirred at room temperature for 30 min then pipetted into Et₂O (75 mL). The precipitate was collected by filtration then dissolved in neat TFA (4 mL). After 20 min, Et₂O (100 mL) was added and the precipitate collected by filtration to provide the title compound as a yellow solid (826 mg, 95% yield).

LC/MS: Calc'd m/z=729.2 for C₃₆H₃₆FN₇O₉, found [M+H]⁺=730.2.

### 4.53: 6-(2,5-dioxo-2,5-dihydro-1H-pyrrol-1-yl)-N-(2-((2-(((S)-1-((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)hexanamide (MC-GGFG-Compound 141)

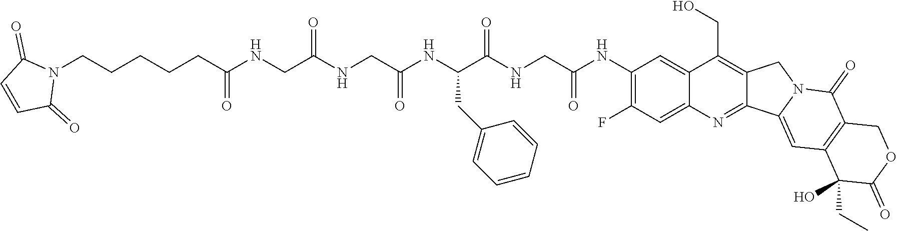 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCCCCN3C(=O)C=CC3=O)cc1c2CO]

To Compound 4.52 (826 mg) was added a solution of 2,5-dioxocyclopentyl 6-(2,5-dioxopyrrol-1-yl)hexanoate (382 mg, 1.1 equiv) and N-ethyldiisopropylamine (300 μL) in DMF (5.5 mL). This solution was stirred at room temperature for 30 min then pipetted into Et₂O (100 mL). The precipitate was collected by filtration then dissolved in DMF (2 mL). Preparative HPLC purification was accomplished as described in General Procedure 9, eluting with a 25 to 40% CH₃CN/H₂O+0.1% TFA gradient to provide the title compound as a yellow solid (370 mg, 35% yield).

LC/MS: Calc'd m/z=922.9 for C₄₆H₄₇FN₈O₁₂, found [M+H]⁺=923.8.

¹H NMR (300 MHz, CD₃CN) δ 8.63 (d, J=8.4 Hz, 1H), 7.67 (d, J=11.9 Hz, 1H), 7.38-7.27 (m, 5H), 7.24 (d, J=4.3 Hz, 1H), 6.72 (s, 2H), 5.48 (d, J=16.4 Hz, 1H), 5.28 (d, J=16.3 Hz, 1H), 5.24-5.01 (m, 4H), 4.65 (dd, J=9.7, 4.9 Hz, 1H), 4.13 (s, 2H), 3.85-3.75 (m, 3H), 3.37 (t, J=7.1 Hz, 2H), 3.00 (dd, J=14.0, 9.8 Hz, 1H), 2.21 (t, J=7.6 Hz, 2H), 1.51 (dp, J=22.0, 7.4 Hz, 4H), 1.22 (p, J=7.4, 7.0 Hz, 2H), 0.94 (t, J=7.3 Hz, 3H).

### 4.54: tert-butyl ((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)carbamate (Compound 4.54)

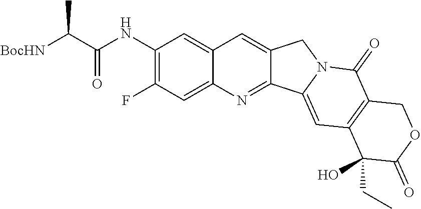 [SMILES: CC[C@@]1(O)C(=O)OCc2c1cc1n(c2=O)Cc2cc3cc(NC(=O)[C@H](C)NC(=O)OC(C)(C)C)c(F)cc3nc2-1]

To Compound 3.4 (500 mg, 1.0 mmol) was added TFA (4 mL) and this solution was allowed to stand at rt for 1 h, then Et₂O (100 mL) was added, and the precipitate was collected by filtration. This solid was taken up in DMF (3.4 mL) and Boc-Ala-OH (590 mg, 3.1 mmol, 3 equiv) and HATU (1.2 g, 3.1 mmol, 3 equiv) were added followed by N-ethyldiisopropylamine (0.9 mL, 5.2 mmol, 5 equiv). This solution was stirred at rt for 3 days then poured into ice water (50 mL) and the precipitate was collected by filtration to give the title compound as a brown solid (125 mg, 22% yield).

LC/MS: Calc'd m/z=552.6 for C₂₈H₂₉FN₄O₇, found [M+H]⁺=553.7.

### 4.55: (S)-2-amino-N—((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)-3-methylbutanamide (Compound 4.55)

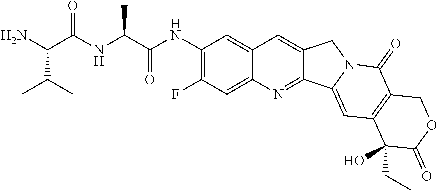 [SMILES: CC[C@@]1(O)C(=O)OCc2c1cc1n(c2=O)Cc2cc3cc(NC(=O)[C@H](C)NC(=O)[C@@H](N)C(C)C)c(F)cc3nc2-1]

To Compound 4.54 (125 mg, 0.225 mmol) in a 100 mL round bottom flask was added TFA (2 mL). This solution was allowed to stand for 10 min, then Et₂O (50 mL) was added, and the precipitate collected by filtration. The resulting orange solid was added to a solution of Boc-Val-NHS (78 mg, 0.25 mmol, 1.1 equiv) and N-ethyldiisopropylamine (80 μL, 0.45 mmol, 2 equiv) in DMF (2 mL). This solution was stirred at rt for 30 min, then pipetted into Et₂O (40 mL) in a 50 mL falcon tube and the precipitate was collected by centrifugation and decanting of the Et₂O. The pellet was dissolved in TFA (2 mL) and allowed to stand for 10 min prior to the addition of Et₂O (40 mL). The precipitate was collected by centrifugation and decanting the Et₂O. The pellet was dried under high vacuum to give the title compound as an orange solid (135 mg, 90% yield over 3 steps).

LC/MS: Calc'd m/z=551.2 for C₂₈H₃₀FN₅O₆, found [M+H]⁺=552.2.

### 4.56: 6-(2,5-dioxo-2,5-dihydro-1H-pyrrol-1-yl)-N—((S)-1-(((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)amino)-3-methyl-1-oxobutan-2-yl)hexanamide (MC-VA-Compound 140)

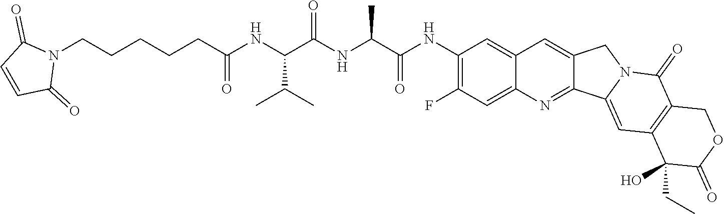 [SMILES: CC[C@@]1(O)C(=O)OCc2c1cc1n(c2=O)Cc2cc3cc(NC(=O)[C@H](C)NC(=O)[C@@H](NC(=O)CCCCCN4C(=O)C=CC4=O)C(C)C)c(F)cc3nc2-1]

To Compound 4.55 (20 mg, 0.03 mmol) was added a solution of 2,5-dioxopyrrolidin-1-yl 6-(2,5-dioxopyrrol-1-yl)hexanoate (11 mg, 0.036 mmol) and N-ethyldiisopropylamine (10 μL) in DMF (1 mL). This solution was stirred at rt for 30 min then purified directly. Preparative HPLC purification was accomplished as described in General Procedure 9, eluting with a 20 to 60% CH₃CN/H₂O+0.1% TFA gradient to provide the title compound as a yellow solid (8.8 mg, 40% yield).

LC/MS: Calc'd m/z=744.8 for C₃₈H₄₁FN₆O₉, found [M+H]⁺=745.6.

¹H NMR (300 MHz, 10% D₂O/CD₃CN) δ 8.60 (d, J=8.5 Hz, 1H), 8.31 (s, 1H), 7.96 (d, J=6.5 Hz, 1H), 7.65 (d, J=12.0 Hz, 1H), 7.37-7.26 (m, 2H), 6.75 (s, 2H), 5.45 (d, J=16.6 Hz, 1H), 5.25 (d, J=16.3 Hz, 1H), 5.04 (d, J=4.0 Hz, 2H), 4.78-4.58 (m, 1H), 4.30-4.13 (m, 1H), 2.32-2.16 (m, 2H), 2.10 (dt, J=13.6, 6.8 Hz, 1H), 1.88 (q, J=7.4 Hz, 2H), 1.57 (dq, J=15.5, 7.6 Hz, 4H), 1.45 (d, J=7.1 Hz, 3H), 1.26 (tt, J=10.1, 6.1 Hz, 2H), 1.05-0.83 (m, 9H).

### 4.57. 2,5-dioxopyrrolidin-1-yl 6-(((S)-1-(((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)amino)-3-methyl-1-oxobutan-2-yl)amino)-6-oxohexanoate (NHC-C-VA-Compound 140)

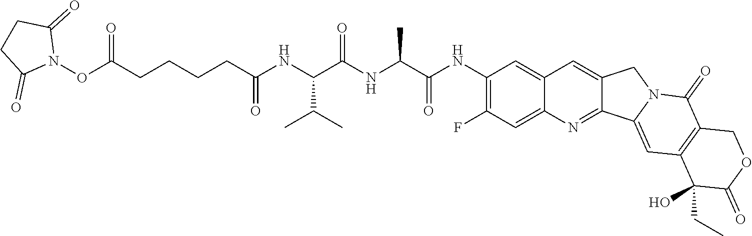 [SMILES: CC[C@@]1(O)C(=O)OCc2c1cc1n(c2=O)Cc2cc3cc(NC(=O)[C@H](C)NC(=O)[C@@H](NC(=O)CCCCC(=O)ON4C(=O)CCC4=O)C(C)C)c(F)cc3nc2-1]

To Compound 4.55 (20 mg, 0.03 mmol) was added a solution of bis(2,5-dioxopyrrolidin-1-yl) adipate (30 mg, 0.09 mmol, 3 equiv) and N-ethyldiisopropylamine (10 μL) in DMF (1 mL). This solution was stirred at rt for 30 min then purified directly. Preparative HPLC purification was accomplished as described in General Procedure 9, eluting with a 25 to 35% CH₃CN/H₂O+0.1% TFA gradient to provide the title compound as a yellow solid (4.1 mg, 18% yield).

LC/MS: Calc'd m/z=776.8 for C₃₈H₄₁FN₆O₁₁, found [M+H]⁺=777.6.

¹H NMR (300 MHz, 10% D₂O/CD₃CN) δ 8.65 (dd, J=8.4, 2.3 Hz, 1H), 8.38 (s, 1H), 7.95 (d, J=6.5 Hz, 1H), 7.72 (d, J=12.0 Hz, 1H), 7.37 (d, J=11.8 Hz, 2H), 5.58-5.19 (m, 2H), 5.10 (s, 2H), 4.78-4.56 (m, 1H), 4.23 (dd, J=8.4, 7.0 Hz, 1H), 2.80 (s, 4H), 2.65 (t, J=6.9 Hz, 2H), 2.39-2.22 (m, 2H), 2.11 (q, J=6.8 Hz, 1H), 1.94-1.81 (m, 2H), 1.79-1.57 (m, 4H), 1.45 (d, J=7.1 Hz, 3H), 1.10-0.78 (m, 9H).

### 4.58: (S)-2-(32-azido-5-oxo-3,9,12,15,18,21,24,27,30-nonaoxa-6-azadotriacontanamido)-N—((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)-3-methylbutanamide (2-((Azido-PEG8-carbamoyl)methoxy)acetamido-VA-Compound 140)

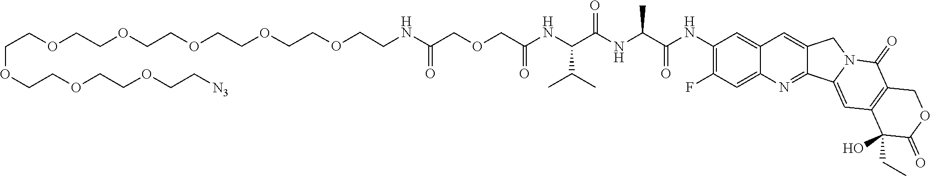 [SMILES: CC[C@@]1(O)C(=O)OCc2c1cc1n(c2=O)Cc2cc3cc(NC(=O)[C@H](C)NC(=O)[C@@H](NC(=O)COCC(=O)NCCOCCOCCOCCOCCOCCOCCOCCOCCN=[N+]=[N-])C(C)C)c(F)cc3nc2-1]

A solution of Compound 4.55 (20 mg, 0.03 mmol), 32-azido-5-oxo-3,9,12,15,18,21,24,27,30-nonaoxa-6-azadotriacontanoic acid (17 mg, 0.03 mmol), and HATU (13 mg, 0.03 mmol) in DMF (300 μL) was cooled to 0° C. and N-ethyldiisopropylamine (16 μL, 0.09 mmol) was added. This solution was stirred for 30 min the purified directly. Preparative HPLC purification was accomplished as described in General Procedure 9, eluting with a 20 to 50% CH₃CN/H₂O+0.1% TFA gradient to provide the title compound as a yellow solid (13.7 mg, 42% yield).

LC/MS: Calc'd m/z=1088.2 for C₅₀H₇₀FN₉O₁₇, found [M+H]⁺=1088.8.

¹H NMR (300 MHz, 10% D₂O/CD₃CN) δ 8.62 (d, J=8.5 Hz, 1H), 8.33 (s, 1H), 7.66 (d, J=12.2 Hz, 1H), 7.35 (s, 1H), 5.52-5.18 (m, 2H), 5.04 (s, 2H), 4.72 (q, J=7.1 Hz, 1H), 4.32 (d, J=7.3 Hz, 1H), 4.15-3.98 (m, 4H), 3.68-3.48 (m, 35H), 3.41-3.34 (m, 6H), 2.18 (h, J=6.8 Hz, 1H), 1.88 (q, J=7.4 Hz, 2H), 1.47 (d, J=7.1 Hz, 3H), 1.13-0.84 (m, 9H).

### 4.58: tert-butyl (2-(pyridin-2-yldisulfaneyl)ethyl)carbamate (Compound 4.58)

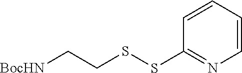 [SMILES: CC(C)(C)OC(=O)NCCSSc1ccccn1]

The title compound was prepared as described in Wang, et al., Nano Lett., 2014, 14(10):5577-5583.

### 4.59: tert-butyl (2-((2-hydroxyethyl)disulfaneyl)ethyl)carbamate (Compound 4.59)

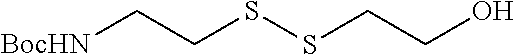 [SMILES: CC(C)(C)OC(=O)NCCSSCCO]

To a solution of Compound 4.58 (200 mg, 0.7 mmol) in DCM (1.4 mL) was added p-mercaptoethanol (50 μL, 0.7 mmol) and this solution was stirred at rt for 5 h. The solution was diluted with DCM (10 mL), washed with a water (3×10 mL), dried over Na₂SO₄, and concentrated to an oil. Purification was accomplished as described in General Procedure 9, using a 10 g silica column, and eluting with a 0 to 10% MeOH/DCM to give the title compound as a colorless solid (212 mg, 82% yield).

LC/MS: Calc'd m/z=253.1 for C₁₁H₂₃NO₃S₂, found [M+H,-Boc]⁺=154.0.

¹H NMR (300 MHz, Chloroform-d) δ 4.94 (s, 1H), 3.91 (t, J=5.7 Hz, 2H), 3.49 (q, J=6.4 Hz, 2H), 2.86 (dt, J=23.7, 6.1 Hz, 4H), 2.15 (s, 2H), 1.47 (s, 9H).

### 4.60: 2-((2-(3-(2,5-dioxo-2,5-dihydro-1H-pyrrol-1-yl)propanamido)ethyl)disulfaneyl)ethyl (4-nitrophenyl) carbonate (Compound 4.60)

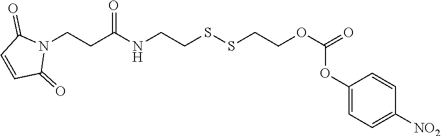 [SMILES: O=C(CCN1C(=O)C=CC1=O)NCCSSCCOC(=O)Oc1ccc([N+](=O)[O-])cc1]

To Compound 4.59 (212 mg, 0.837 mmol) in a 25 mL round bottom flask was added a 4M HCl/dioxane solution (5 mL) and the solution was stirred at rt for 30 min, then evaporated to dryness. The residue was suspended in EtOAc (10 mL) and evaporated to dryness to give the amine as the HCl salt and as a white powder. To this solid was added a solution of 2,5-dioxopyrrolidin-1-yl 3-(2,5-dioxopyrrol-1-yl)propanoate (245 mg, 0.92 mmol, 1.1 equiv.) and N-ethyldiisopropylamine (0.438 mL, 2.51 mmol) in DMF (1.7 mL). This solution was stirred at rt for 20 min then 4-nitrophenyl carbonate (280 mg, 0.92 mmol) was added and the reaction was then left to stir overnight. Purification of the crude reaction mixture was accomplished as described in General Procedure 9, using a 12 g C18 flash column, and eluting with a 10 to 100% CH₃CN/H₂O+0.1% TFA gradient to provide the title compound as a white solid (141 mg, 36% yield).

LC/MS: Calc'd m/z=469.5 for C₁₈H₁₉N₃O₈S₂, found [M+H]⁺=470.2.

¹H NMR (300 MHz, Chloroform-d) δ 8.37-8.25 (m, 2H), 7.46-7.35 (m, 2H), 6.71 (d, J=2.1 Hz, 2H), 6.32 (s, 1H), 4.55 (t, J=6.6 Hz, 2H), 3.83 (t, J=7.0 Hz, 2H), 3.65-3.50 (m, 2H), 3.09-2.99 (m, 2H), 2.84 (q, J=6.1 Hz, 2H), 2.52 (td, J=7.1, 3.1 Hz, 2H).

### 4.61: 2-((2-(3-(2,5-dioxo-2,5-dihydro-1H-pyrrol-1-yl)propanamido)ethyl)disulfaneyl)ethyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (DiS-Compound 145)

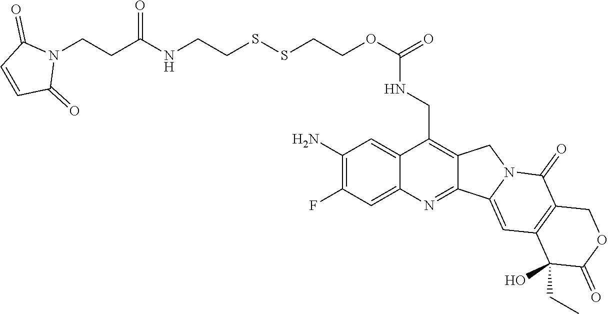 [SMILES: CC[C@@]1(O)C(=O)OCc2c1cc1n(c2=O)Cc2c-1nc1cc(F)c(N)cc1c2CNC(=O)OCCSSCCNC(=O)CCN1C(=O)C=CC1=O]

A solution of Compound 4.60 (18 mg, 0.038 mmol) and N-ethyldiisopropylamine (15 μL, 0.087 mmol) in DMF (300 μL) was added to Compound 145 (13 mg, 0.029 mmol) and this solution was stirred at rt for 20 min. The solution was acidified with an aqueous 1M HCl solution (100 μL) and purified directly. Preparative HPLC purification was accomplished as described in General Procedure 9, eluting with a 20 to 45% CH₃CN/H₂O+0.1% TFA gradient to provide the title compound as a yellow solid (6.8 mg, 32% yield).

LC/MS: Calc'd m/z=740.8 for C₃₃H₃₃FN₆O₉S₂, found [M+H]⁺=741.5.

¹H NMR (300 MHz, 10% D₂O/CD₃CN) δ 7.63 (d, J=12.1 Hz, 1H), 7.39-7.22 (m, 2H), 6.74 (d, J=6.7 Hz, 2H), 5.50 (d, J=16.2 Hz, 1H), 5.26 (d, J=16.2 Hz, 1H), 5.20 (s, 2H) 4.69 (s, 2H), 4.28 (t, J=6.3 Hz, 2H), 3.62 (t, J=7.0 Hz, 2H), 3.31 (t, J=6.6 Hz, 2H), 2.74-2.64 (m, 2H), 2.35 (t, J=7.0 Hz, 2H), 1.90 (dd, J=15.5, 8.1 Hz, 2H), 1.23-1.04 (m, 6H), 0.93 (t, J=7.4 Hz, 3H).

### Example 5: In Vitro Cytotoxicity of Camptothecin Analogues

Cytotoxicity of the camptothecin analogues was assessed in vitro as follows.

In vitro potency was assessed on multiple cancer cell lines: SK-BR-3 (breast cancer), SKOV-3 (ovarian cancer), Calu-3 (lung cancer), ZR-75-1 (breast cancer) and MDA-MB-468 (breast cancer). Serial dilutions of camptothecin analogues were prepared in RPMI 1640+10% FBS, and 20 μL of each dilution was added to 384-well plates. Cells cultured in log-phase growth were detached by brief incubation in 0.05% Trypsin and resuspended in respective culturing media at 20,000 cells/mL (with the exception of ZR-75 cells, which were resuspended at 10,000 cells/mL). 50 μL of cell suspension was then added to the plates containing test articles. Cells were incubated with test articles for 4 d at 37° C. (with the exception of ZR-75 cells, which were incubated for 5 d). Growth inhibition was assessed by CellTiter-Glo® (Promega Corporation, Madison, WI) and luminescence was measured on a plate reader. IC50 values were determined by GraphPad Prism (GraphPad Software, San Diego CA).

The results are shown in Table 5.1.

TABLE 5.1
In vitro Cytotoxicity of Camptothecin Analogues (pIC50)

| | pIC50 | | | | |
|---|---|---|---|---|---|
| Camptothecin | Calu- | SKBR- | SKOV- | ZR- | MDA- |
| Analogue | 3 | 3 | 3 | 75 | MB-468 |
| Compound 100 | 8.95 | 8.96 | 8.64 | 8.91 | 8.60 |
| Compound 102 | ND* | 9.03 | 8.67 | 9.10 | 8.63 |
| Compound 104 | 8.28 | 8.57 | 8.11 | 8.59 | 8.58 |
| Compound 122 | 8.43 | 8.95 | 8.04 | 8.89 | 8.96 |
| Compound 132 | 7.29 | 7.64 | 6.98 | 7.81 | 7.57 |
| Compound 106 | 8.34 | 8.47 | 8.13 | 8.42 | 8.03 |
| Compound 108 | 7.39 | 7.53 | 7.26 | 7.73 | 7.32 |
| Compound 101 | ND | 8.97 | 8.80 | 8.97 | 8.59 |
| Compound 103 | 8.63 | 8.77 | 8.36 | 8.62 | 8.33 |
| Compound 105 | 8.35 | 8.44 | 7.97 | 8.51 | 8.56 |
| Compound 107 | 8.39 | 8.51 | ND | 8.56 | 8.41 |
| Compound 109 | ND | 8.24 | 8.15 | 8.30 | 8.09 |
| Compound 134 | 7.61 | 8.02 | 7.11 | 7.92 | 7.94 |
| Compound 138 | 9.41 | 9.32 | 9.21 | 9.71 | 9.06 |
| Compound 124 | 8.77 | 8.92 | ND | 8.64 | 8.46 |
| Compound 136 | 7.58 | 8.40 | 7.29 | 8.30 | 8.20 |
| Compound 133 | 7.92 | 8.17 | 7.13 | 8.01 | 7.97 |
| Compound 135 | 7.60 | 7.91 | ND | 7.88 | 7.97 |
| Compound 137 | 7.20 | 7.77 | ND | 7.73 | 7.55 |
| Compound 123 | 8.43 | 8.72 | 7.71 | 8.61 | 8.70 |
| Compound 125 | 8.55 | 8.88 | 8.15 | 8.88 | 8.42 |
| Compound 128 | ND | 8.28 | 7.74 | 8.37 | 8.08 |
| Compound 126 | 8.53 | 8.96 | 8.20 | 9.14 | 8.71 |
| Compound 130 | 8.78 | 7.83 | 7.62 | ND | 8.19 |
| Compound 129 | 8.28 | 8.51 | 7.69 | 8.58 | 8.40 |
| Compound 127 | 8.03 | 8.48 | 7.84 | 8.28 | 8.42 |
| Compound 110 | 9.09 | 8.88 | 9.03 | 9.07 | 8.81 |
| Compound 111 | 7.71 | 8.07 | 7.78 | 8.04 | 7.54 |
| Compound 112 | 7.76 | 8.18 | 7.70 | 7.92 | 7.87 |
| Compound 113 | 8.52 | 8.71 | 8.37 | 8.66 | 8.47 |
| Compound 139 | 8.15 | 8.92 | 8.30 | 8.97 | 8.82 |
| Compound 140 | 9.51 | 9.50 | 9.34 | 9.48 | 9.15 |
| Compound 141 | 8.99 | 9.46 | 8.55 | 9.51 | 8.84 |
| Compound 142 | 8.89 | 9.20 | 8.89 | 9.12 | 8.89 |
| Compound 143 | 9.15 | 9.41 | 8.55 | 9.07 | 9.15 |
| Compound 144 | 7.65 | 9.10 | 7.16 | 7.88 | 7.65 |
| Compound 145 | 9.57 | 9.45 | 9.03 | 9.42 | 9.57 |
| Compound 146 | 8.36 | 8.76 | 7.95 | 8.22 | 8.36 |
| Compound 147 | 7.67 | 8.29 | 7.36 | ND | 7.67 |
| Compound 148 | 9.69 | 9.49 | ND | 9.66 | 9.69 |
| Compound 131 | 8.04 | 8.98 | ND | 9.02 | 8.04 |
| Compound 149 | 8.20 | 8.50 | 8.00 | 8.74 | 8.20 |
| Compound 150 | ND | 8.10 | 7.20 | 8.19 | ND |
| Compound 151 | 7.94 | 8.57 | 7.34 | 8.23 | 7.94 |
| Compound 152 | 8.83 | 8.49 | 8.54 | ND | 8.30 |
| Compound 153 | 9.94 | 9.29 | 9.03 | ND | ND |
| Compound 114 | 10.03 | 9.97 | 9.75 | ND | 9.38 |
| Compound 115 | 8.89 | 8.59 | 8.42 | ND | 8.38 |
| Compound 117 | 9.79 | 10.02 | 9.77 | ND | 9.33 |
| Compound 118 | 9.03 | 8.82 | 8.84 | ND | 8.73 |
| Compound 119 | 8.93 | 8.38 | 8.43 | ND | 8.56 |
| Compound 120 | 10.00 | 8.96 | 9.60 | ND | 9.72 |
| Compound 121 | 9.84 | 9.83 | 9.71 | ND | 9.57 |
| Compound 116 | 9.10 | 8.15 | 7.90 | ND | 8.03 |

*ND = not determined

### Example 6: Preparation of Anti-NaPi2B Antibodies

Antibody constructs that specifically bind human NaPi2b were generated by immunizing mice with human cells over-expressing NaPi2b as summarized below.

HEK293-6E cells (National Research Council of Canada) were transiently transfected with a pTT5-based expression plasmid (National Research Council of Canada) encoding human NaPi2b (pTT5-huNaPi2b, expressing the sequence of NaPi2b as set forth in SEQ ID NO:1), according to manufacturer's instructions for Lipofectamine 2000 (Thermo Fisher Scientific). Ten B6x129 mice were subcutaneously immunized with transfected HEK293-6E cells over 63 days, after which blood was drawn and spleens harvested.

Anti-human NaPi2b antibody titers were determined by flow cytometry using CHO-S cells expressing human NaPi2b. All ten mice mounted a significant response against human NaPi2b.

Splenocytes from all mice were subsequently pooled and used for hybridoma generation. P3X63Ag8.653 cells (ATCC cat #CRL-1580) were mixed with IgG+B cells isolated from spleens and fused using an ECM 2001 electrofusion instrument (BTX, Harvard Bioscience) with optimized settings. Following overnight recovery, hybridomas were diluted and plated in selection media containing the following final concentrations of substituents: 100 M hypoxanthine, 0.4 M aminopterin and 16 μM thymidine. Following 14 days of selection, hybridomas were diluted to an average of one cell per well and plated into 96-well plates. Cell supernatants containing secreted antibodies were assessed for binding on CHO-S cells transfected with the same plasmid used to transfect HEK293-6E cells (pTT5-huNaPi2b). Hybridoma cells from wells containing supernatants having antibodies that bound NaPi2b were harvested for sequencing.

The murine VH and VL sequences for one of the anti-NaPi2b antibodies identified were used to prepare a mouse-human chimeric IgG1/kappa antibody construct, v23855, as follows. Coding sequences for antibody variable regions were cloned in frame into a human IgG1 expression vector (with human IgG1 constant region starting with alanine 118 according to Kabat numbering) or a human C kappa expression vector (with human C kappa constant region starting at arginine 108 according to Kabat numbering), both expression vectors based on pTT5. The activities of the resultant recombinant chimeric antibody construct were confirmed in specificity binding assays and were found comparable to the parental antibodies (data not shown).

### Example 7: Humanization of Anti-NaPi2B Antibody Constructs

The chimeric anti-human NaPi2b antibody construct, variant v23855, generated as described in Example 6, was humanized. The CDR sequences of v23855 are provided in Table 7.1, and the mouse VH and VL sequences are provided in Table 7.2. Humanization was conducted as described below.

TABLE 7.1
CDR Sequences of the Anti-NaPi2b Antibody Construct v23855

| Numbering | | | | | | |
|---|---|---|---|---|---|---|
| System | Sequence | | | | | |
| | | SEQ | | SEQ | | SEQ |
| | Heavy Chain | ID | Heavy Chain | ID | Heavy Chain | ID |
| | CDR1 | NO | CDR2 | NO | CDR3 | NO |
| IMGT | GFTFTSYN | 4 | ISPGNGDL | 5 | ARGRTGMGAVDY | 6 |
| Kabat | SYNVH | 7 | AISPGNGDLS | 8 | GRTGMGAVDY | 9 |
| | | | YNQRFKD | | | |
| Chothia | GFTFTSY | 10 | SPGNGD | 11 | GRTGMGAVDY | 9 |
| AbM | GFTFTSYNVH | 12 | AISPGNGDLS | 13 | GRTGMGAVDY | 9 |
| Contact | TSYNVH | 14 | WIAAISPGNG | 15 | ARGRTGMGAVD | 16 |
| | | | DLS | | | |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QDVYSY | 17 | YTS | — | QQYNIFPWT | 18 |
| Kabat | RASQDVYSYLN | 19 | YTSRLQS | 20 | QQYNIFPWT | 18 |
| Chothia | RASQDVYSYLN | 19 | YTSRLQS | 20 | QQYNIFPWT | 18 |
| AbM | RASQDVYSYLN | 19 | YTSRLQS | 20 | QQYNIFPWT | 18 |
| Contact | YSYLNWY | 21 | LLIYYTSRLQ | 22 | QQYNIFPW | 23 |

TABLE 7.2
VH and VL Sequences of the Anti-NaPi2b Antibody Construct v23855

| | | SEQ ID |
|---|---|---|
| | Sequence | NO |
| VH | QAYLQQSGAELVRPGASVKMSCKASGFTFTSYNVHWVKQTPRQGLEWI | 31 |
| | AAISPGNGDLSYNQRFKDKATLTVDRSSSTAYMQLSGLTSEDSAVYFCAR | |
| | GRTGMGAVDYWGQGTSVTVSS | |
| VL | DVQMTQTASSLSASLGDRVTISCRASQDVYSYLNWYQQKPHGTVKLLIY | 32 |
| | YTSRLQSGVPSRFTGSGSGTDYSLTISNLEPEDIATYYCQQYNIFPWTFGG | |
| | GTKLEIK | |

### 7.1 Humanization

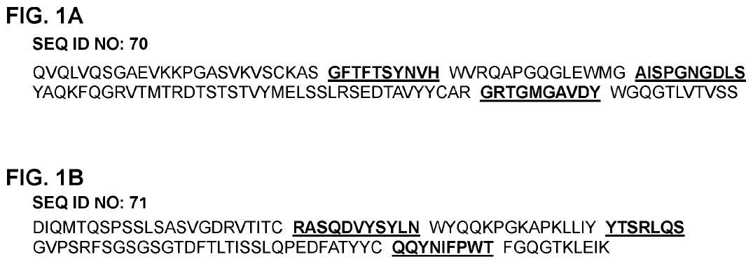

Sequence alignment of the mouse VH and VL sequences of v23855 to respective human germline sequences identified IGHV1-46*03 and IGKV1D-39*01 as the closest, as well as most frequent, human germline sequences (and respectively IGHJ4*03 and IGKJ2*04 joining region germline sequences were selected). CDR sequences according to the AbM definition (see Table 7.1) were ported onto the framework of these selected human germline sequences as shown in FIG. 1. Back mutations to mouse residues in the resultant sequences at positions judged likely to be important for the retention of binding affinity to antigen, NaPi2b, were included creating several humanized sequences in which generated sequences were built on the previous sequence, and where the first humanized sequence contained no back mutations. None of the variants modified the CDRs of the parent antibody as defined by the AbM method.

This process resulted in four variable heavy chain humanized sequences and three variable light chain humanized sequences. Full heavy chain sequences containing humanized heavy chain variable domain (VH) and hIgG1 heavy chain constant domains (CH1, hinge, CH2, CH3), and full light chain sequence containing humanized light chain variable domain (VL) and human kappa light chain constant domain (kappa CL) were assembled. Monoclonal antibody (mAb) variants were then assembled such that each of the humanized heavy chains was paired with each of the humanized light chains to provide 12 humanized variants to be evaluated experimentally.

### 7.2 Production of Humanized Antibody Constructs

Each of the 12 humanized antibody constructs, as well as the parental v23855 construct (e.g., FIGS. 1A-1B), were produced in full-size antibody (FSA) format containing two identical full-length heavy chains and two identical kappa light chains.

The full-length heavy chain contained the human CH1-hinge-CH2-CH3 domain sequence of IGHG1*01 [SEQ ID NO:33]; see Table 7.3). The light chain contained human kappa CL sequence of IGKC*01 [SEQ ID NO:34]; see Table 7.3).

TABLE 7.3
Human Constant Heavy and Light Chain Sequences

| | | SEQ ID |
|---|---|---|
| | Sequence | NO |
| CH1-hinge- | ASTKGPSVFPLAPSSKSTSGGTAALGCLVKDYFPEPVTVSWNSGALTS | 33 |
| CH2-CH3 | GVHTFPAVLQSSGLYSLSSVVTVPSSSLGTQTYICNVNHKPSNTKVDK | |
| (IGHG1*01) | KVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRD | |
| | ELTKNQVSLTCLVKGFYPSDIAVEWESNGQPENNYKTTPPVLDSDGSF | |
| | FLYSKLTVDKSRWQQGNVFSCSVMHEALHNHYTQKSLSLSPG | |
| CL (kappa) | RTVAAPSVFIFPPSDEQLKSGTASVVCLLNNFYPREAKVQWKVDNALQ | 34 |
| (IGKC*01) | SGNSQESVTEQDSKDSTYSLSSTLTLSKADYEKHKVYACEVTHQGLSS | |
| | PVTKSFNRGEC | |

Each of the humanized VH domain sequences as well as the mouse VH domain sequence was appended to the human CH1-hinge-CH2-CH3 domain sequence of IGHG1*01 to provide four humanized full heavy chain sequences and one parental mouse-human chimeric full heavy chain sequence. Each of the humanized VL domain sequences or mouse VL domain sequence was appended to the human kappa CL sequence of IGKC*01 to provide three humanized light chain sequences and one parental mouse-human chimeric light chain sequence. All sequences were reverse translated to DNA, codon optimized for mammalian expression and gene synthesized.

Heavy chain vector inserts comprising a signal peptide (artificially designed sequence: MRPTWAWWLFLVLLLALWAPARG [SEQ ID NO:35](Barash et al., 2002, Biochem and Biophys Res. Comm., 294:835-842)) and the heavy chain clone terminating at residue G446 (EU numbering) of the CH3 domain were ligated into a pTT5 vector to produce heavy chain expression vectors. Light chain vector inserts comprising the same signal peptide were ligated into a pTT5 vector to produce light chain expression vectors. The resulting heavy and light chain expression vectors were sequenced to confirm correct reading frame and sequence of the coding DNA. Sequences of the humanized VH and VL sequences are provided in Table 7.4 below.

TABLE 7.4
Amino Acid Sequences of Humanized VH and VL Sequences

| | | SEQ ID |
|---|---|---|
| Name | Amino Acid Sequence | NO |
| H1 | QVQLVQSGAEVKKPGASVKVSCKASGFTFTSYNVHWVRQAPGQ | 24 |
| | GLEWMGAISPGNGDLSYAQKFQGRVTMTRDTSTSTVYMELSSL | |
| | RSEDTAVYYCARGRTGMGAVDYWGQGTLVTVSS | |
| H2 | QVQLVQSGAEVKKPGASVKVSCKASGFTFTSYNVHWVRQAPGQ | 25 |
| | GLEWIAAISPGNGDLSYAQKFQGRVTMTVDTSTSTVYMELSSLR | |
| | SEDTAVYYCARGRTGMGAVDYWGQGTLVTVSS | |
| H3 | QVQLVQSGAEVKKPGASVKMSCKASGFTFTSYNVHWVRQAPG | 26 |
| | QGLEWIAAISPGNGDLSYAQKFQGRATLTVDTSTSTAYMELSSLR | |
| | SEDTAVYYCARGRTGMGAVDYWGQGTLVTVSS | |
| H4 | QVQLVQSGAEVKKPGASVKMSCKASGFTFTSYNVHWVRQAPG | 27 |
| | QGLEWIAAISPGNGDLSYAQKFQGRATLTVDRSTSTAYMELSSL | |
| | RSEDTAVYYCARGRTGMGAVDYWGQGTLVTVSS | |
| L1 | DIQMTQSPSSLSASVGDRVTITCRASQDVYSYLNWYQQKPGKAP | 28 |
| | KLLIYYTSRLQSGVPSRFSGSGSGTDFTLTISSLQPEDFATYYCQQ | |
| | YNIFPWTFGQGTKLEIK | |
| L2 | DVQMTQSPSSLSASVGDRVTITCRASQDVYSYLNWYQQKPGKA | 29 |
| | VKLLIYYTSRLQSGVPSRFSGSGSGTDYTLTISSLQPEDFATYYCQ | |
| | QYNIFPWTFGQGTKLEIK | |
| L3 | DVQMTQSPSSLSASVGDRVTITCRASQDVYSYLNWYQQKPGKA | 30 |
| | VKLLIYYTSRLQSGVPSRFSGSGSGTDYTLTISSLQPEDIATYYCQ | |
| | QYNIFPWTFGQGTKLEIK | |

The heavy and light chains of each of the humanized antibody variants and parental mouse-human chimeric antibody variant were expressed in 300 mL cultures of CHO-3E7 cells. Briefly, CHO-3E7 cells, at a density of 1.7-2×10⁶ cells/mL, viability >95%, were cultured at 37° C. in FreeStyle™ F₁₇ medium (Thermo Fisher Scientific, Waltham, MA) supplemented with 4 mM glutamine (Hyclone SH30034.01) and 0.1% Pluronic® F-68 (Gibco/Thermo Fisher Scientific, Waltham, MA). A total volume of 300 mL CHO-3E7 cells+1× antibiotic/antimycotics (GE Life Sciences, Marlborough, MA) was transfected with a total of 300 g DNA (150 g of antibody DNA and 150 g of GFP/AKT/stuffer DNA) using PEI-MAX® (Polyscience, Inc., Philadelphia, PA) at a DNA:PEI ratio of 1:4 (w/w). Twenty-four hours after the addition of the DNA-PEI mixture, 0.5 mM valproic acid (final concentration)+100 w/v Tryptone (final concentration) were added to the cells, which were then transferred to 32° C. and incubated for 6 more days prior to harvesting.

Protein-A purification was performed using 1 mL HiTrap™ MabSelect™ SuRe™ columns (Cytiva, Marlborough, MA). Clarified supernatant samples were loaded on equilibrated columns in Dulbecco's PBS (DPBS). The columns were washed with DPBS. Proteins were eluted with 100 mM sodium citrate buffer pH 3.0. The eluted fractions were pH adjusted by adding 10% (v/v) 1M HEPES (pH~10.6-10.7) to yield a final pH of 6-7. Samples were buffer exchanged into DPBS using 5 mL Zeba™ Spin columns (Thermo Scientific). Protein was quantitated based on absorbance at 280 nm (A280 nm).

Following purification, purity of samples was assessed by SDS-PAGE under non-reducing and reducing conditions. Protein sample was mixed with NuPAGE® LDS Sample Buffer and NuPAGE® Sample Reducing Agent (for reducing condition only) according to manufacturer's protocol, after which sample was heated at 70° C. for 15 min. Treated protein samples containing 1.5 μg of protein and Molecular Weight (MW) Precision Plus Protein™ Dual Color (Bio-Rad) standards for MW estimation were loaded on the NuPAGE 4-12% Bis-Tris gel (15 wells). XCell SureLock© Mini-Cell system from Life Technologies (Thermo Fisher Scientific) as well as NuPAGE® MOPS SDS Running Buffer, were used to perform gel electrophoresis at 200 V for 50 minutes. Gels were stained with a Biosafe Coomassie solution and ChemiDoc™ MP Imaging system (Bio-Rad) was used to capture images of gels.

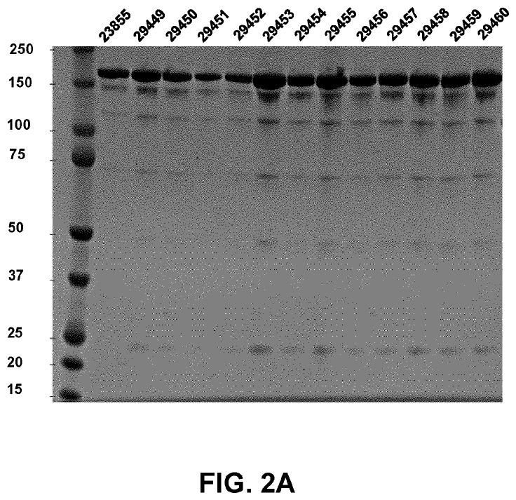

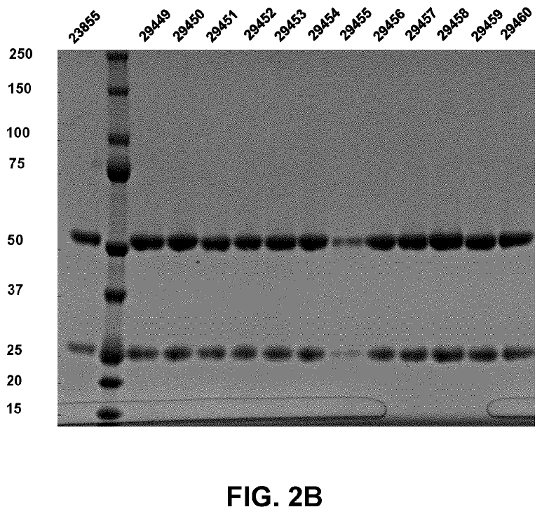

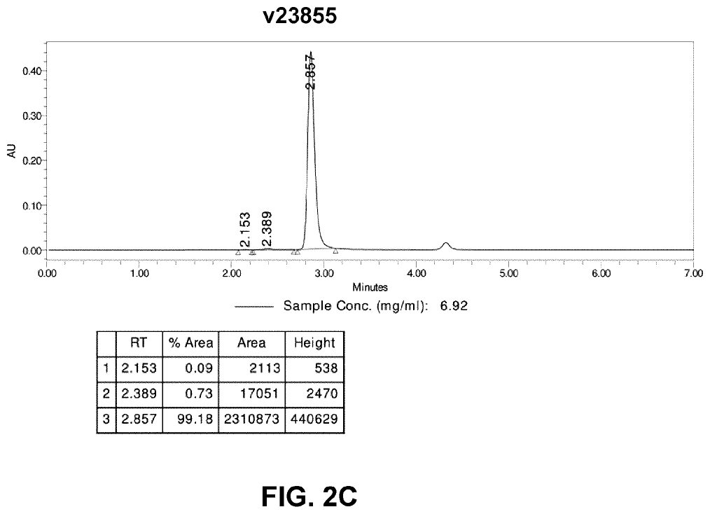

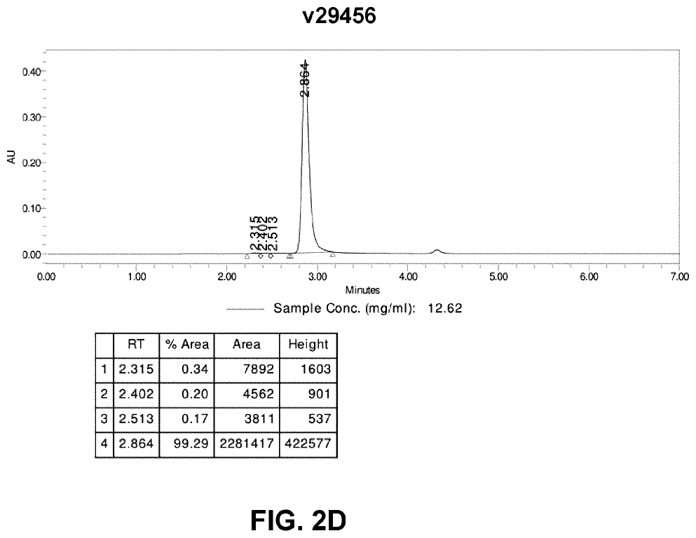

The yields for each of the twelve humanized antibody variants were similar, ranging from approximately 23-30 mg (or ~77-100 mg/L of culture) and approximately 2-fold higher yield than the parental mouse-human chimeric antibody, v23855 (14 mg yield). The SDS-PAGE results for these antibody samples are shown in FIG. 2A (non-reducing) and FIG. 2B (reducing). As can be seen from these Figures, non-reducing (NR) and reducing (R) SDS-PAGE reflected a single species corresponding to full-size antibody and intact heavy and light chains.

### 7.3 Quality Assessment of Humanized Antibodies

Species homogeneity of the humanized antibody variants and parental mouse-human chimeric antibody variant samples was assessed by UPLC-SEC after protein-A purification.

UPLC-SEC was performed using a Waters Acquity BEH200 SEC column (2.5 mL, 4.6×150 mm, stainless steel, 1.7 m particles) (Waters LTD, Mississauga, ON) set to 30° C. and mounted on a Waters Acquity UPLC™ H-Class Bio system with a photodiode array (PDA) detector. The mobile phase was Dulbecco's phosphate buffered saline (DPBS) with 0.02% Tween 20 pH 7.4 and the flow rate was 0.4 mL/min. Total run time for each injection was 7 min with a total mobile phase volume of 2.8 mL. Elution was monitored by UV absorbance in the range 210- 500 nm, and chromatograms were extracted at 280 nm. Peak integration was performed using Waters Empower® 3 software employing the Apex Track™ and detect shoulders features.

FIG. 2C and FIG. 2D show the UPLC-SEC profiles for the parental mouse-human chimeric antibody v23855 and representative humanized antibody v29456 samples. The UPLC-SEC profile for the representative humanized antibody sample reflected high species homogeneity, comparable to the parental mouse-human chimeric antibody sample. The samples from the remaining humanized antibody variants had similar profiles to that shown for the representative humanized antibody sample.

### 7.4 Purity Assessment of Humanized Antibodies

The apparent purity of the humanized antibody variants was assessed using mass spectrometry and non-denaturating deglycosylation.

10 μg of each sample were incubated with 1 μg of deglycosylation mix (NEB, P6044) for 1 hour at room temperature and transferred to the incubator at 37° C. for 16 hours.

After deglycosylation, 5 μL of the eluted sample were transferred into glass inserts in LC-MS vials. For LC-MS analysis, 1 μL of sample were injected into using an Agilent PLRP-S column (1000 Å, 2.1×50 mm, 8 μm) using an Agilent 1290 Infinity II LC system coupled to Agilent 6545 QTOF with Dual Jet Stream electrospray ionization source with a column temperature of 70° C. and a flow rate of 0.3 mL/mi. Mobile phases consisted of A: LC-MS grade water with 0.1% v/v formic acid, 0.025 v/v trifluoroacetic acid and 10% v/v isopropyl alcohol in, and B: acetonitrile with 0.1% v/v formic acid and 10% v/v isopropyl alcohol. The column was pre-equilibrated in 20% mobile phase B before injection. Then, a 20 minute 20 to 40% mobile phase B gradient was applied, followed by a 2 minute 27 to 90% mobile phase B gradient and a column wash of 2 minutes at 99% mobile phase B.

### Example 8: Characterization of Humanized Anti-NaPi2B Antibody Constructs—Assessment of Thermal Stability

The thermal stability of the humanized antibody variants was assessed by differential scanning calorimetry (DSC) as described below.

400 μL of purified samples primarily at concentrations of 0.4 mg/mL in PBS were used for DSC analysis with a VP-Capillary DSC (Malvern Panalytical Inc., Westborough, MA). At the start of each DSC run, 5 buffer blank injections were performed to stabilize the baseline, and a buffer injection was placed before each sample injection for referencing. Each sample was scanned from 20° C. to 100° C. at a 60° C./hr rate, with low feedback, 8 sec filter, 3 min pre-scan thermostat, and 70 psi nitrogen pressure. The resulting thermograms were referenced and analyzed using Origin 7 software (OriginLab Corporation, Northampton, MA) to determine melting temperature (Tm) as an indicator of thermal stability.

The Fab Tm values determined for the humanized variants are shown in Table 8.1. All humanized variants exhibited increased thermal stability compared to the parental antibody, v23855 (Fab Tm of ~72.4° C.), with Fab Tm values ranging from ~78-83° C.

TABLE 8.1
Thermal Stability of Humanized Variants

| | Heavy and Light | Fab Tm |
|---|---|---|
| Variant | Chain Composition | (° C.) |
| v23855 | chimera | 72.4 |
| (parental) | | |
| v29449 | H4L3 | 80.0 |
| v29450 | H3L3 | 81.0 |
| v29451 | H2L3 | 83.0 |
| v29452 | HIL3 | 79.5 |
| v29453 | H4L2 | 78.9 |
| v29454 | H3L2 | 79.7 |
| v29455 | H2L2 | 81.5 |
| v29456 | H1L2 | 77.9 |
| v29457 | H4L1 | 80.5 |
| v29458 | H3L1 | 81.6 |
| v29459 | H2L1 | 83.1 |
| v29460 | H1L1 | 80.3 |

### Example 9: Functional Characterization of Anti-NaPi2B Antibody Constructs—Competition Binding (Epitope Binning)

To characterize the binding of parental chimeric anti-NaPi2b antibody v23855 to NaPi2b, competition binding or epitope binning assays were carried out against anti-NaPi2b reference antibodies MX-35 (v18992) and lifastuzumab (v18993). Binding was assessed by flow cytometry using HEK293-6e cells as described below.

Each of the anti-NaPi2b detection antibodies: v23855, v18992, v18993, and Palivizumab (anti-RSV, v16955) was conjugated with AF647 fluorophores using Zenon Human IgG labeling kit (ThermoFisher Scientific Corporation, Waltham, MA; Cat. No. Z25408 Lot. No. 1937175). HEK293-6e cells were transfected for ~24 hours to transiently express human NaPi2b (1 μg of pTT5-NaPi2b per 1 million cells) or transfected with GFP (ATUM, Menlo Park, CA; pD2610-v23, also 1 μg of DNA per 1 million cells). Following transfection, human NaPi2b-expressing HEK296-6e cells and transfected GFP-expressing HEK296-6e cells were mixed at 4:1 ratio. Each well of a V-bottom 96-well plate was seeded with 100,000 cells of the mixture and incubated with 100 μg/mL of unlabeled competitor anti-NaPi2b antibody for an hour on ice. Post incubation, cells were washed and stained with 1 μg/mL of AF647-conjugated anti-NaPi2b detection antibodies for an hour on ice. Following staining and washing, fluorescence was detected by flow cytometry on a BD LSRFortessa™ Cell Analyzer (BD Biosciences, Franklin Lake, NJ) with 10,000 minimum events collected per well. The AF647/APC-A GeoMean (fluorescence signal geometric mean, proportional to anti-Human AF647 binding) was calculated for the FITC/GFP negative live cell population and using FlowJo™ Version 10.8.1 (BD Biosciences, Franklin Lake, NJ). Percentage inhibition was calculated using the following formula:

$$\% inhibition of detection mAb = 100 - ( \frac{Competitor mAb GeoMean}{v 16955 GeoMean} * 100 )$$

The competition binding results of parental chimeric anti-NaPi2b antibody v23855 against v18992 (MX35) and v18993 (lifastuzumab) are shown in Table 9.1.

TABLE 9.1
Competition binding. Percentage inhibition (vs. negative control antibody). GFP-population

| | | Labeled detection antibody (1 μg/mL) | | | |
|---|---|---|---|---|---|
| | | | | | v16955 |
| % Inhibition vs. irrelevant | | | v18992 | v18993 | (negative |
| mAb | | v23855 | (MX35) | (lifastuzumab) | control |
| Unlabeled | v23855 | 96 | 97 | 94 | −148 |
| competitor | v18992 | 95 | 97 | 95 | −135 |
| antibody | (MX35) | | | | |
| (100 μg/mL) | v18993 | 94 | 94 | 96 | −89 |
| | (lifastuzumab) | | | | |
| | v16955 | 0 | 0 | 0 | 0 |
| | (negative | | | | |
| | control) | | | | |
| | No mAb | −20 | −1 | −14 | −17 |

Each of the tested anti-NaPi2b antibodies competed against itself (>95% inhibition, see data in bold text) as expected. The chimeric anti-NaPi2b antibody v23855 competed for binding to v18992 and v18993, demonstrated by comparable % inhibition to itself (>94%). No competition binding against negative control palivizumab (v16955) was observed, as expected.

### Example 10: Functional Characterization of Humanized Anti-NaPi2B Antibody Constructs—Human, Cynomolgus and Mouse NaPi2B Binding

The binding cross-reactivity of humanized antibody variant v29456 to human, cynomolgus and mouse NaPi2b was assessed by flow cytometry using HEK293-6e transfected cells. Reference anti-NaPi2b antibodies MX-35 (v18992) and lifastuzumab (v18993) were included as comparators and the anti-RSV antibody palivizumab (v22277) was included as a negative control.

Briefly, HEK293-6e cells were transfected for ~24 hours to transiently express human NaPi2b, cynomolgus or mouse NaPi2b, at 1 μg of DNA per 1 million cells. Following transfection, 50,000 cells per well were seeded in V-bottom 96-well plates and incubated with 200 nM of primary antibody for 18-24 hours at 4° C. to prevent internalization. Post-incubation, cells were washed and stained with anti-Human IgG Fc AF647 conjugate (Jackson Immuno Research Labs, West Grove, PA, Cat. No. 109-605-098, Lot. No. 124868) at 4° C. for 1 hour. Following staining and washing, fluorescence was detected by flow cytometry on BD LSRFortessa™ Cell Analyzer (BD Biosciences, Franklin Lake, NJ) with 10,000 minimum events collected per well. The AF647/APC-A GeoMean (fluorescence signal geometric mean, proportional to anti-Human AF647 binding) was calculated for the live singlet cell population for each primary antibody using FlowJo™ v8 software (BD Biosciences, Franklin Lake, NJ). The Bmax and Kd of each primary antibody were calculated using GraphPad Prism Version 9 (GraphPad Software, San Diego, CA)

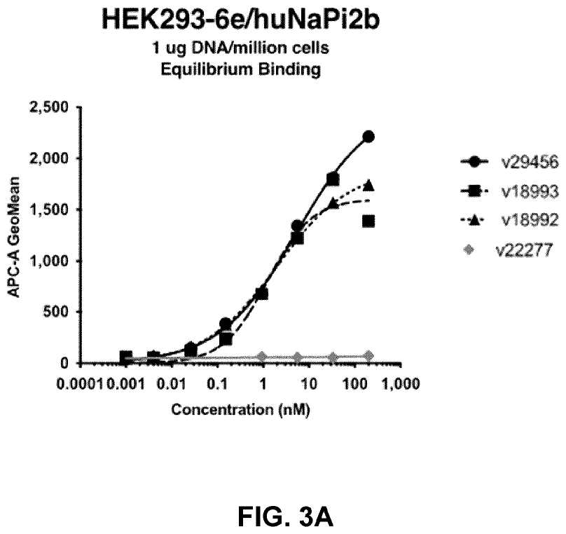

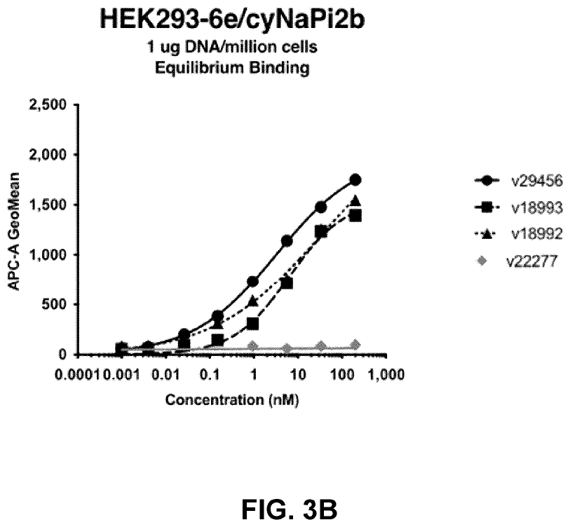

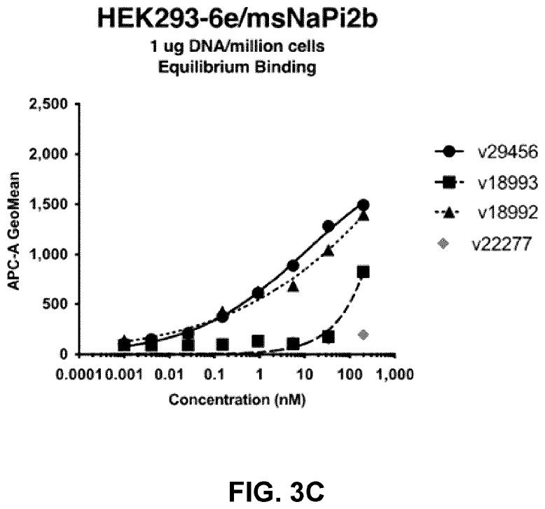

The binding results of humanized antibody variant v29456, MX-35 (v18992) and lifastuzumab (v18993) are shown in Table 10.1 and FIG. 3A (human NaPi2b), FIG. 3B (cynomolgus NaPi2b), and FIG. 3C (mouse NaPi2b).

TABLE 10.1
Cross-reactivity of v29456 to cynomolgus and mouse NaPi2b

| | Bmax | | | Apparent Kd (nM) | | |
|---|---|---|---|---|---|---|
| | Hu | Cyno | Ms | Hu | Cyno | Ms |
| Sample | NaPi2b | NaPi2b | NaPi2b | NaPi2b | NaPi2b | NaPi2b |
| v29456 | 2,538 | 2,006 | 2,062 | 5.20 | 3.20 | 10.59 |
| (H1L2) | | | | | | |
| v18992 | 1,896 | 2,365 | 3,930 | 1.98 | 31.03 | >200* |
| (MX35) | | | | | | |
| v18993 | 1,607 | 1,557 | N/A | 1.22 | 6.27 | N/A |
| (lifastuzumab) | | | | | | |
| v22277 | N/A | N/A | N/A | N/A | N/A | N/A |
| (palivizumab) | | | | | | |

*Apparent Kd value greater than the highest antibody testing concentration (>200 nM)

v29456 and v18992 showed binding to human, cynomolgus and mouse NaPi2b on transfected HEK296-6e cells. Lifastuzumab (v18993) displayed binding to human and cynomolgus NaPi2b and showed minimal binding to mouse NaPi2b transfected HEK293-6e cells.

v29456 had comparable apparent Kd value to v18992 and v18993 on human NaPi2b binding and exhibited the greatest binding to cynomolgus NaPi2b, yielding apparent Kd 10-fold and 2-fold lower than v18992 (MX35) and v18993 (lifastuzumab), respectively. Negative control palivizumab (v22277) did not bind to any species tested, as expected.

### Example 11: Functional Characterization of Anti-NaPi2B Antibody Constructs—Cell-Binding of Monovalent Antibodies by Kinexa

The binding affinity of anti-NaPi2b antibody constructs was assessed by Kinexa in the endogenous NaPi2b-expressing cell line, IGROV-1. Affinity of antibody constructs was assessed in monovalent format to reduce the effects of avidity and internalization and compared directly to the parental chimeric antibody variant, also in monovalent format. v29814 (monovalent format of parental chimeric variant 23855), v36123 (monovalent format of humanized antibody variant 29452, and v36124 (monovalent format of humanized antibody variant 29456) were assessed. The experiment was performed as described below.

### IGROV-1 Cell Preparation:

IGROV-1 cells were cultured in RPMI 1640 Medium, ATCC modification (Thermo Fisher Scientific, Waltham, MA) supplemented with 10% FBS (Thermo Fisher Scientific, Waltham, MA) in T175 culture flasks (Corning, Corning, NY) and incubated at 37° C. with 5% CO₂ until achieving 80% confluency. Cells were detached from culture vessels by incubation with Cell Dissociation Buffer (Invitrogen, Waltham, MA) for 30-60 minutes at 37° C. with 5% CO₂, collected by neutralizing Cell Dissociation Buffer with at least 5 times volume of RPMI 1640 Medium, ATCC modification supplemented with 10% FBS, and maintained on ice until use. Cells were counted using the Vi-Cell™ XR Cell Viability Analyzer (Beckman Coulter, Brea, California).

### KinExA Preparation:

The solid phase was prepared by coating one vial of PMMA (polymethyl methacrylate) beads (Sapidyne, Boise, Idaho) with 1 mL of 20 μg/mL BSA-biotin (Sigma-Aldrich, St. Louis, Missouri) in PBS pH 7.4. The beads were incubated for 2 hours at room temperature with gentle rotation. Beads were settled, supernatant removed, and beads rinsed five times with PBS pH 7.4. The beads were then coated with 1 mL of 100 μg/mL of streptavidin (Jackson ImmunoResearch, West Grove, PA) in PBS pH 7.4 with 10 mg/mL BSA (Sigma-Aldrich, St. Louis, Missouri) with rotation at room temperature for 1 hour. Beads were settled, supernatant removed, and beads rinsed five times with PBS pH7.4. The final step of the solid phase preparation was done by coating with 30 μg/mL of biotin goat anti-human IgG (Jackson ImmunoResearch, West Grove, PA) for 1 hour with rotation at room temperature.

### Cell-Binding Assay:

The cell-binding assay was set up using the antibody constructs or variants as constant binding partner at two different concentrations of 50 μM and 500 μM. For the titration curve with antibody variants fixed at 50 μM, at least 10 million IGROV-1 cells were used as titrant. For the titration curve with antibody variants fixed at 500 μM, at least 5 million cells were used as titrant. The antibody variants and cells were mixed in PBS pH 7.4, 1 mg/mL BSA, 0.2% NaN₃ and incubated at 4° C. for 7 days with gentle rotation until equilibrium was reached. After incubation, the mixture of antibody variants and cells was centrifuged to separate the cells from the unbound free antibody variants. The free antibody variants were loaded onto the KinExA 3200 (Sapidyne, Boise, Idaho) with biotinylated-anti-human IgG PMMA as solid phase and 0.5 μg/mL of Alexa 647 goat anti-human IgG (Jackson ImmunoResearch, West Grove, PA) as detection antibody.

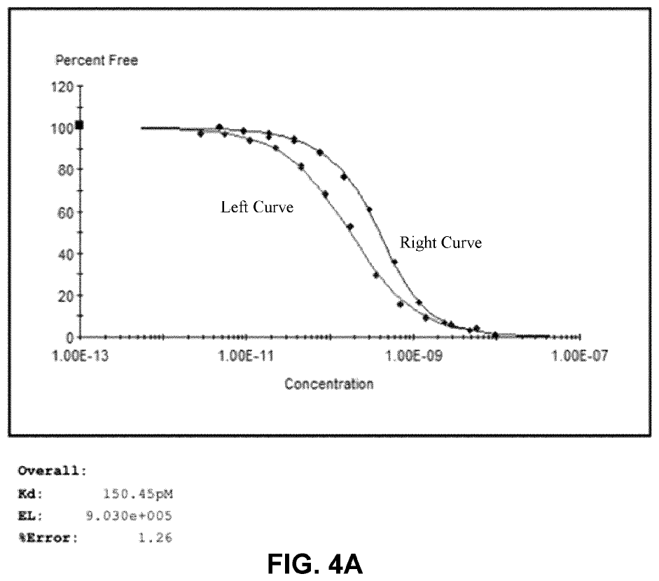

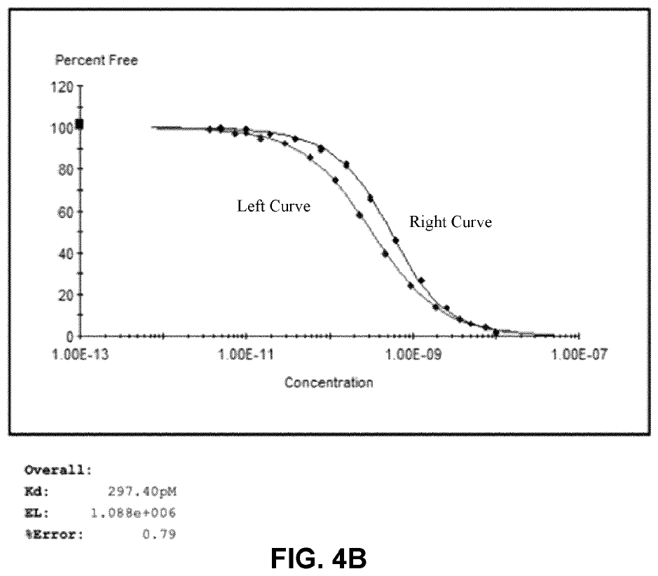

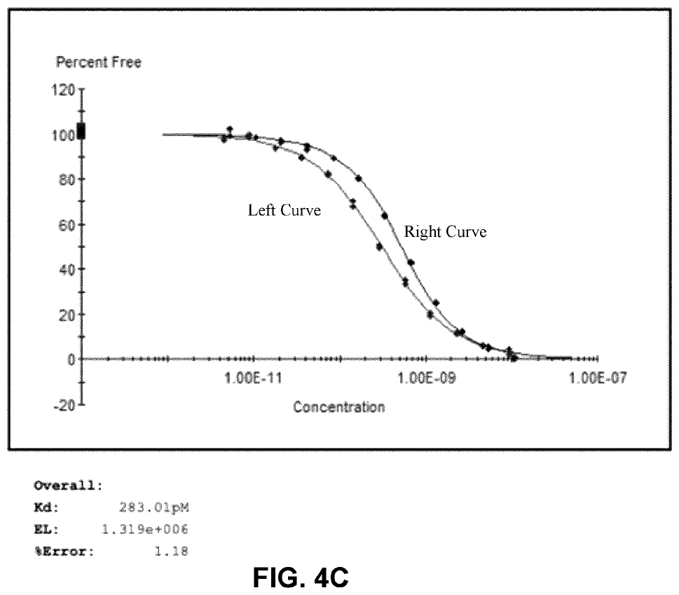

Results for parental chimeric monovalent antibody variant (v29814) and two humanized, monovalent antibody variants are shown in Table 11.1. N-curve analysis was used to calculate the affinity and receptor expression level with the concentrations of antibody variant as reference point. Narrow 95% confidence intervals were obtained for both the affinity and receptor expression level and % error of the fit was less than 1.5%. The N-curve analysis is shown in FIG. 4A (v29814), FIG. 4B (v36123), and FIG. 4C (v36124). For each panel, the right curve shows the data for 500 μM constant binding partner and the left curve shows the data for 50 μM constant binding partner.

TABLE 11.1
Binding of Anti-Napi2b Antibody Constructs to IGROV-1 cells

| Variant/ | | | % |
|---|---|---|---|
| Construct | KD ^{#} | Expression Level ^{#} | Error |
| v29814 | 150.45 pM | 9.01E+5 | 1.26 |
| (parental | (202.2 pM-114.3 | (1.16E+6-7.34E+5) | |
| chimeric) | pM) | | |
| v36123 | 297.4 pM | 1.088E+6 | 0.79 |
| (VH1/VL3) | (379 pM-238.7 pM) | (1.37E+6-8.95E+5) | |
| v36124 | 283.01 pM | 1.32E+6 | 1.18 |
| (VH1/VL2) | (405.7 pM-207.8 | (1.89E+6-1.0E+6) | |
| | pM) | | |

^{#} Parentheses indicates 95% confidence interval

All humanized anti-NaPi2b antibody variants displayed similar binding profiles to each other and approximately 2-fold lower binding affinity compared to chimeric parental antibody construct. The calculated receptor expression level was between 0.9-1.3 million per cell.

### Example 12: Functional Characterization of Anti-NaPi2B Antibody Constructs—Internalization

Internalization of the chimeric parental anti-NaPi2b antibody v23855 and a representative humanized variant, v29456 (H1L2), in NaPi2b-expressing cell lines (HCC-78 and NCI-H441) was determined by flow cytometry as described below. The NaPi2b-targeting antibodies lifastuzumab (v18993) and MX35 (v18992) were used as positive controls, and the anti-RSV antibody palivizumab (v22277) was used as a negative control.

Briefly, antibodies were fluorescently labeled by coupling to a Fab fragment AF488 conjugate targeting anti-Human IgG Fc (Jackson Immuno Research Labs, West Grove, PA; Cat. No. 109-547-008) at a 1:1 molar ratio in PBS pH 7.4 (Thermo Fisher Scientific, Waltham, MA; Cat. No. 10010-023), for 24 hours at 4° C. Cells were seeded at 50,000 cells/well in RPMI 1640, ATCC modification (Thermo Fisher Scientific, Waltham, MA) supplemented with 10% fetal bovine serum (Thermo Fisher Scientific, Waltham, MA) in 48-well plates and incubated overnight under standard culturing conditions (37° C./5% CO₂) to allow attachment. Coupled antibodies were added to cells the following day at 10 nM and incubated under standard culturing conditions for 5-24 hours to allow for internalization. Following incubation, cells were dissociated, washed, and surface AF488 fluorescence was quenched using an anti-AF488 antibody (Life Technologies, Carlsbad, CA; Cat. No. A-11094) at 100 nM for 30 minutes at 4° C. Quenched AF488 fluorescence (internalized fluorescence) was detected by flow cytometry on a BD LSRFortessa™ Cell Analyzer (BD Biosciences, Franklin Lake, NJ) with 1,000 minimum events collected per well. The AF488/FITC-A GeoMean (fluorescence signal geometric mean, proportional to anti-Human Fab AF488 labelling) was calculated for the live single cell population using FlowJo™ Version 10.8.1 (BD Biosciences, Franklin Lake, NJ) and plotted using GraphPad Prism Version 9 (GraphPad Software, San Diego, CA).

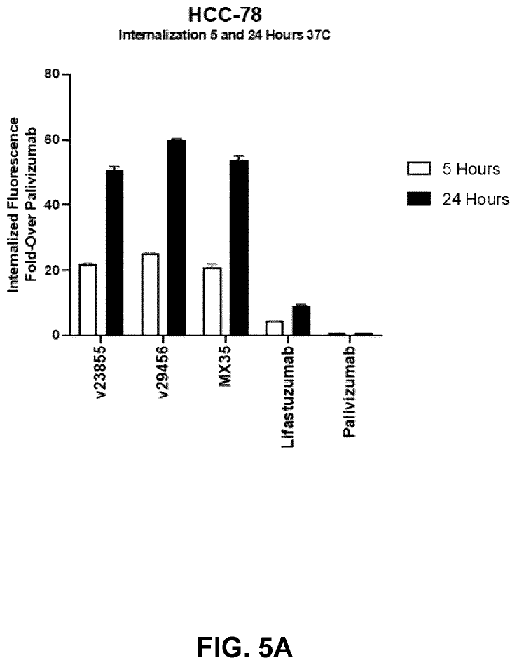

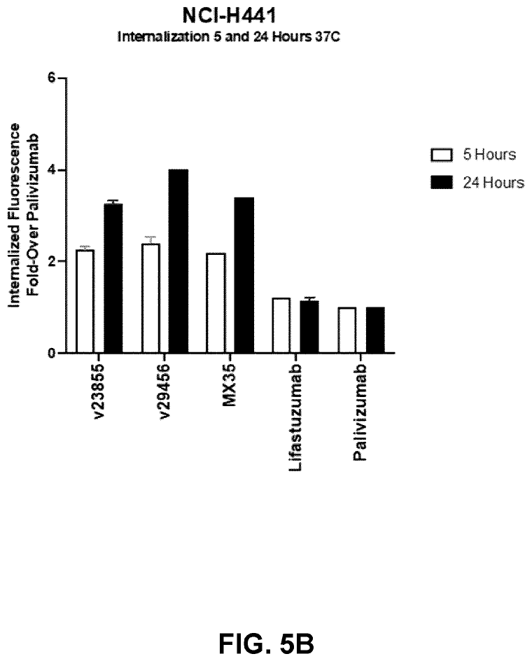

Results are shown in FIG. 5A (HCC-78) and FIG. 5B (NCI-H441), and in Table 12.1 below.

TABLE 12.1
Internalization of Antibody Constructs

| | Internalized Fluorescence | | | |
|---|---|---|---|---|
| | Fold-Over Palivizumab | | | |
| | HCC-78 | | NCI-H441 | |
| Antibody (10 nM) | 5 hrs | 24 hrs | 5 hrs | 24 hrs |
| v23855 (parental | 21.9 | 50.9 | 2.2 | 3.3 |
| chimeric) | | | | |
| v29456 (H1L2) | 25.3 | 59.9 | 2.4 | 4.0 |
| v18992 (MX35) | 21.1 | 53.9 | 2.2 | 3.4 |
| v18993 | 4.6 | 9.1 | 1.2 | 1.2 |
| (lifastuzumab) | | | | |
| v22277 (neg control) | 1.0 | 1.0 | 1.0 | 1.0 |

Chimeric parental antibody v23855 and the humanized antibody variant v29456 showed comparable levels of internalization to the humanized antibody MX35 (v18992) and much greater levels of internalization compared to humanized antibody lifastuzumab (v18993) across all time points (5 hours and 24 hours) at 10 nM antibody treatment on both HCC-78 and NCI-H441 cells. For instance, following a 5-hour incubation in HCC-78, v23855 and v29456 showed 21.9 and 25.3-fold increase in internalized fluorescence compared to negative control palivizumab, respectively. Similarly, following a 24-hour incubation in HCC-78 cells, v23855 and v29456 showed 50.9- and 59.9-fold increase in internalized fluorescence compared to negative control palivizumab, respectively.

### Example 13: Developability Assessment of Anti-NaPi2B Antibodies

The isoelectric point, propensity for self-aggregation, and non-specific binding of anti-NaPi2b antibodies v23855 (parental chimeric), v29452 (H1L3) and v29456 (H1L2), were determined in order to assess the developability of these antibodies. The isoelectric point was measured by capillary isoelectric focusing (cIEF), the propensity for self-aggregation was measured by Affinity-capture self-interaction nanoparticle spectroscopy (AC-SINS) and non-specific binding was measured by NS-ELISA, as described below.

Capillary Isoelectric Focusing (cIEF)

cIEF was carried out using Maurice C. (ProteinSimple©) system, System Suitability Kit and Method Development Kit. System suitability standard, fluorescence calibration standard, cartridge and samples were prepared according to vendor's recommendations. The capillary was automatically calibrated with a fluorescence standard preconditioned with Maurice cIEF System Suitability Kit to ensure the capillary was functioning properly. The antibody samples were diluted to a concentration of 0.5 mg/mL in a final volume of 40 μL in Gibco™ Distilled Water, and mixed Maurice cIEF Method Development Kit Samples. The samples were then vortexed, centrifuged and the supernatant pipetted into individual wells of a 96-well plate. All electropherograms were detected with UV absorbance at 280 nm. All data analyses were performed using vendor software Compass for iCE (ProteinSimple©). The Compass software aligns each electropherogram using the pI markers so that the x-axis is displayed as a normalized pI for each injection.

### AC-SINS Assay

AC-SINS method was carried out in a 384-well plate format (Corning® #3702). Initially, 20 nm gold nanoparticles (Ted Pella, Inc., #15705) washed with 0.22 μm filtered Gibco™ Distilled Water were coated with a mixture of capture antibody-80% AffiniPure Goat Anti-Human IgG (H+L) (Jackson ImmunoResearch Laboratories© #109-005-088), and the non-capture antibody- 20% ChromPure Goat IgG, whole molecule (Jackson ImmunoResearch Laboratories© #005-000-003), that were initially buffer exchanged into 20 mM sodium acetate pH 4.3 and diluted to 0.4 mg/mL. The mixture of gold nanoparticles, capture antibody and non-capture antibody was incubated in the dark for 18 h at room temperature. Sites unoccupied on the gold nanoparticles were blocked with 1 μM thiolated polyethylene glycol (2 kD) in 20 mM sodium acetate, pH 4.3 to a final concentration of 0.1 μM, followed by 1 h incubation at room temperature. The coated nanoparticles were then concentrated by centrifugation at 21,000×g for 7 min, at 8° C. 95% of the supernatant was removed and the gold pellet was resuspended in the remaining buffer. 5 μL of concentrated nanoparticles were added to 45 μL of antibody at 0.05 mg/mL in Gibco™ PBS pH 7.4 in a 384-well plate. The coated nanoparticles were incubated with the antibody of interest for 4 h at room temperature in the dark. The absorbance was read from 450-700 nm at 1 nm increments, and a Microsoft Excel macro was used to identify the max absorbance, smooth the data, and fit the data using a second-order polynomial. The Δlambda (nm) was calculated based on the smoothed max absorbance of the average blank (PBS alone) subtracted from the smoothed max absorbance of the antibody sample to determine the antibody AC-SINS score. Antibody-antibody interactions directly correlate with the shift in maximum absorbance wavelength of gold nanoparticles coated with the antibody of interest. The cutoff of Δlambda 10 nm was set as high self-aggregation propensity of the antibody.

### NS-ELISA

NS-ELISA was used to measure the propensity of the antibodies to bind to a range of biomolecules to emulate the undesirable non-specific interactions to biological matrices in vivo as described below.

NS-ELISA was carried out in Corning® 96-well EIA/RIA Easy Wash™ Clear Flat Bottom Polystyrene High Bind Microplate coated overnight at 4° C. with 50 μL of Heparin (Sigma, H3149) diluted with 50 mM sodium carbonate pH 9.6 to a final concentration of 250 μg/mL. The plate was incubated for 2 days at room temperature, wells that were coated with heparin were not covered to allow air dry. Insulin (Sigma-Aldrich®, 19278) and KLH (Sigma-Aldrich®, H8283) were each diluted with 50 mM sodium carbonate pH 9.6 to a final concentration of 5 μg/mL. ssDNA (Sigma-Aldrich®, D8899) and dsDNA (Sigma-Aldrich®, D4553) was diluted with Gibco™ PBS pH7.4 to a final concentration of 10 μg/mL. 50 μL each of insulin, KLH, dsDNA and ssDNA were added to a 96 well plate, followed by the incubation at 37° C. for 2 h. The coating materials were removed, and the plate was blocked with 200 μL of Gibco™ PBS pH7.4, 0.1% Tween®20, and incubated for 1 h at room temperature with shaking at 200 rpm. The plate was washed 3 times with Gibco™ PBS pH7.4, 0.1% Tween 20. 50 μL of each mAb at 100 nM (15 mg/mL) in Gibco™ PBS pH 7.4, 0.1% Tween®20 was added in duplicate to the wells and incubated for 1 h at room temperature with shaking at 200 rpm. Plates were washed three times with Gibco™ PBS pH7.4, 0.1% Tween 20, and 50 μL of 50 ng/mL anti-human IgG HRP (Thermofisher Scientific©, H10307) was added to each well. Plates were incubated for 1 h at room temperature, with shaking at 200 rpm. The plate was washed three times with Gibco™ PBS pH7.4, 0.1% Tween 20, and 100 μL of TMB substrate (Cell Signaling Technology©, 7004P6) added to each well. Reactions were stopped after approximately 10 minutes by adding 100 μL of 1 M HCl to each well, and absorbance was read at 450 nm. Binding scores were calculated as the ratio of the ELISA signal of the antibody to the signal of a well containing buffer instead of the primary antibody. The cutoffs considered for each binding molecule (ssDNA. KLH, Insulin, dsDNA and Heparin) were internally calculated, based on the average of Zymeworks Inc. produced antibodies and antibodies benchmarks published in the literature.

The results of all three assays are shown in Table 13.1. In these assays a score higher than the cutoff was taken to indicate potentially less desirable biophysical characteristics.

TABLE 13.1
Developability assessment results for anti-NaPi2b antibodies

| | | AC-SINS | | | | | |
|---|---|---|---|---|---|---|---|
| | cIEF | Δlambda | NS-ELISA | | | | |
| Sample | pI | (nm) | ssDNA | KLH | Insulin | dsDNA | Heparin |
| v23855 | 8.35 | 2.5 | 1.59 | 2.38 | 1.23 | 1.42 | 1.27 |
| v29452 | 8.53 | 2.0 | 1.22 | 1.34 | 1.03 | 1.0 | 1.05 |
| v29456 | 8.53 | 2.5 | 1.06 | 1.37 | 1.05 | 1.07 | 1.06 |

The pI values determined for the main isoform for the variants v23855; v29452 and v29456 are 8.35, 8.53 and 8.53 respectively, which all fall within the typical range for therapeutic antibodies. The analysis of Δlambda showed no potential issues on AC-SINS for all variants. Additionally, there were no potential issues identified for NS-ELISA.

### Example 14: Stability of a Humanized Antibody Variant in Mouse Plasma or in PBS

The objective of this experiment was to assess if fragmentation of antibody variant v29456 and reference antibody MX35 (v18992) occurred over time after incubation in mouse plasma or in PBS pH 7.4 at 37° C.

Briefly, v29456 or v18992 were each diluted into either PBS or mouse plasma to a final concentration of 0.5 mg/ml and incubated at 37° C. Samples were removed after 0, 7 and 14 days and stored at −80° C. until characterization. For characterization, samples were thawed at room temperature and 50 μg were incubated with 5 μg of recombinant EndoS endoglycosidase for one hour at room temperature. An immunoprecipitation slurry was generated by a 45 min incubation of 95 μL/sample of magnetic Sepharose streptavidin-coated beads with 15 μg/sample of biotinylated goat anti-Human IgG Fc capture antibody, followed by 4 washes with PBS pH 7.4 with the aid of a DynaMag™-2 magnet (Invitrogen™)

After deglycosylation, plasma-incubated samples were incubated with 95 μL of the immunoprecipitation slurry for 1.5 hrs at room temperature. Then, the slurry underwent 6 washes with PBS pH 7.4 and 2 washes with LC-MS-grade water with the aid of a DynaMag™-2 magnet (Invitrogen™). A final wash with PBS pH 7.4 was performed before elution. Protein was eluted by incubating beads with 35 μL of LC-MS grade water with 20% acetonitrile and 0.1% formic acid for one hour at room temperature. PBS samples were not immunoprecipitated.

5 μL of the eluted sample were transferred to glass inserts in LC-MS vials. For LC-MS analysis, 1 μL of sample were injected into a Waters™ BioSuite Phenyl Column, 1000 Å, 10 μm, 4.6 mm×75 mm using a Waters™ ACQUITY™ UPLC I-Class HPLC system coupled to a Waters™ Synapt™ G2-Si HDMS with a column temperature of 70 C and a flow rate of 0.3 mL/min. Mobile phases consisted of A: LC-MS grade water with 0.1% v/v formic acid, 0.025 v/v trifluoroacetic acid and 10% v/v isopropyl alcohol in, and B: acetonitrile with 0.1% v/v formic acid and 10% v/v isopropyl alcohol. The column was pre-equilibrated in 10% mobile phase B before injection. Then, a 20 min 10 to 27% mobile phase B gradient was applied, followed by a 2 min 27 to 90% mobile phase B gradient and a column wash of 2 min at 99% mobile phase B.

The column was re-equilibrated to 10% mobile phase B for 2 minutes between runs. ESI was performed in positive mode with 3 kV of capillary voltage, 120 C source temperature, 100 V sampling cone voltage, source offset of 80 V, source gas flow of 0 ml/min, desolvation temperature 500 C, cone gas flow 0 L/Hr, desolvation gas flow 800 L/hr, nebuliser gas flow 6.5 bar. Data format was continuum with analyser set in sensitivity mode, with a m/z range from 500 to 7000.

Peak integration, MS deconvolution and mass assignments were performed in Protein Metrics Byos® v4.0 using a deconvolution window of 60000-160000 Da with an m/z range of 1000-4000. For all time points, the highest intensity deconvoluted mass was assigned as the reference mass of v29456. The reference mass was defined as the average mass of v29456 or v18992 with two 2-acetamido-2-deoxy-beta-D-glucopyranose-(1-4)-[alpha-L-fucopyranose-(1-6)] stubs arising from EndoS activity on N-glycans, 16 disulfide bonds and the formation of pyroglutamic acid, if applicable, at the N-terminus. Mass assignments had a mass tolerance of ±10 Da. Other assigned mAb proteoforms were: mAb reference mass with a phosphoric acid adduct, mAb reference mass with the loss of one fucose unit, mAb reference mass with the addition of a hexose unit. A pentasaccharide adduct (likely penta-mannose) relative to the reference mass of v29456 without 2-acetamido-2-deoxy-beta-D-glucopyranose-(1-4)-[alpha-L-fucopyranose-(1-6)] was also identified. Apparent purity was calculated as the ratio of all v29456 or v18992 mAb proteoforms deconvoluted peak intensities divided by all observed deconvoluted peak intensities. Mouse plasma proteins present in the day 0 mouse plasma control, but not observed in the PBS controls, were not considered in the apparent purity calculations.

The data is provided in Table 14.1 and shows that there was no evidence of fragmentation of v29456 incubated at 37° C. for 7 and 14 days in mouse plasma or PBS pH 7.4, as apparent purity after 7 and 14 days was similar to day 0 controls. v18992 showed fragmentation after 14 days in PBS based on appearance of low molecular weight species and a decrease in apparent purity of >10% relative to day 0 control. v18992 fragmentation was not observed in plasma.

TABLE 14.1
Stability of Antibodies in Mouse Plasma or PBS

| | v29456 | | v18992 | |
|---|---|---|---|---|
| | | Mouse | | Mouse |
| Timepoint | PBS | Plasma | PBS | Plasma |
| (days) | 37° C. | 37° C. | 37° C. | 37° C. |
| 0 | 99.8% | 99.9% | 95.5% | 96.0% |
| 7 | 99.2% | 100% | 95.6% | 92.3% |
| 14 | 99.6% | 98.4% | 84.3% | 98.6% |

### Example 15—Quantification of Surface NaPi2B Protein on Tumor Cells

Surface NaPi2b protein was measured on tumor cell lines by quantitative flow cytometry using a set of beads with known levels of antibody binding capacity (ABC) as described below. Reference humanized antibody MX35 (v18992) conjugated to Alexa Fluor® AF647 and control anti-RSV antibody palivizumab (v21995) conjugated to Alexa Fluor® AF647 were used to fluorescently label tumor cells and beads. Variant 22277 differs from anti-RSV antibody v21995 used in previous examples in that it has a heterodimeric Fc. This does not affect the function of this antibody. The representative cell lines evaluated were OVCAR-3, IGROV-1, HCC-78, TOV-21G, NCI-H441, HCT116, and EBC-1.

Conjugations of v18992 and v21995 to Alexa Fluor® AF647 were performed as follows: v18992 and v21995 were each reacted with 8 equivalents of NHS-AF647 (Thermo Fisher #A20006, 10 mM) in PBS. The reaction was protected from light at room temperature, and was allowed to proceed for 200 and 150 minutes, respectively. Following incubation, the reactions were purified through four and two rounds of purification, respectively, using a 40 kDa Zeba column (Thermo Fisher, Waltham, MA) pre-equilibrated with PBS, pH 7.4. Confirmation of conjugation and quantification of unconjugated NHS-AF647 were measured by SEC chromatography (Ex: 650 nm, Em: 665 nm).

Cells were detached from culture vessels using Cell Dissociation Buffer (Invitrogen, Waltham, MA) and seeded at 50,000 cells/well in conical-bottom 96-well plates in triplicate. Cells and anti-Human QSC® beads (Bangs Laboratories, Inc., Fishers, IN) were stained with v18992-AF647 with a pre-determined excess level of conjugated antibody or negative control v21995-AF647 at the same concentration and incubated for 30 minutes at 4° C. Following incubation, cells and beads were washed in FACS buffer and analyzed on the BD™ Fortessa HTS and processed using FlowJo™ v8 software (BD Biosciences, Franklin Lake, NJ).

The median AF647 fluorescence intensity for all bead populations were plotted against relevant ABC values using Bangs Laboratories QuickCal v 2.3 calibration line template for QSC® anti-Human IgG Lot #14490.

Surface protein expression for cell lines was calculated based on a monovalent binding model and therefore equivalent to the background subtracted ABC (SABC). The median fluorescence intensity of v21995-AF647-stained cells from each of the respective cell lines was used as a background value for determination of SABC.

Results are shown in Table 15.1. Reported NaPi2b proteins per cell are an average of at least two biological replicates. Tumor cell lines were designated with high, mid, low, or negative expression of the target; cell lines were designated as “high” expressors if the average number of NaPi2b proteins detected was greater than 900,000 per cell; “mid” if the number was between 40,000 and 900,000 per cell; “low” if the number was between 500 and 40,000 per cell; and
“negative” if the number was negative (below limit of quantitation of the calibration beads).

TABLE 15.1
Surface NaPi2b quantification on tumor cell lines

| | | Average | | |
|---|---|---|---|---|
| | | NaPi2b/ | % | |
| Cell Line | Tissue and Disease | cell | CV* | Designation |
| OVCAR-3 | Ovarian adenocarcinoma | 2,340,000 | 18% | High |
| IGROV-1 | Ovarian carcinoma | 1,770,000 | 41% | High |
| HCC-78 | Lung carcinoma (NSCLC) | 970,000 | 48% | High |
| TOV-21G | Ovarian adenocarcinoma | 442,000 | 51% | Mid |
| NCI-H441 | Lung adenocarcinoma | 40,000 | 9% | Mid |
| HCT-116 | Colorectal carcinoma | 1,270 | 183% | Low |
| EBC-1 | Lung carcinoma (NSCLC) | <0 | −333% | Negative |

*Percent coefficient of variation calculated across two to seven biological replicate measurements

### Example 16: Functional Characterization of Anti-NaPi2B Antibody Constructs—Cellular Binding of Bivalent Antibodies by Flow Cytometry

The ability of the parental chimeric antibody construct v23855 and the humanized antibody variants described in Examples 7 and 8 to bind to NaPi2b expressed on cells was assessed on the endogenous NaPi2b-expressing cell line, IGROV-1, by flow cytometry. IGROV-1 cells express endogenous NaPi2b at a high level.

Briefly, cells were seeded at 50,000 cells/well in conical-bottom 96-well plates and treated with test antibody for 24 hours at 4° C. to prevent internalization. Palivizumab (anti-RSV antibody, v22277) was included as a negative control. Reference anti-NaPi2b antibodies lifastuzumab (v18993) and MX35 (v18992) conjugated to a maleimide functionalized auristatin drug linker (DL2) were included as comparators; conjugation of this drug linker has shown no impact to the antibody binding capability (data not shown). Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal geometric mean, proportional to anti-Human AF647 binding) was calculated for the live cell population using FlowJo™ Version 10.8.1 (BD Biosciences, Franklin Lake, NJ) and plotted for each test antibody using GraphPad Prism Version 9 (GraphPad Software, San Diego, CA).

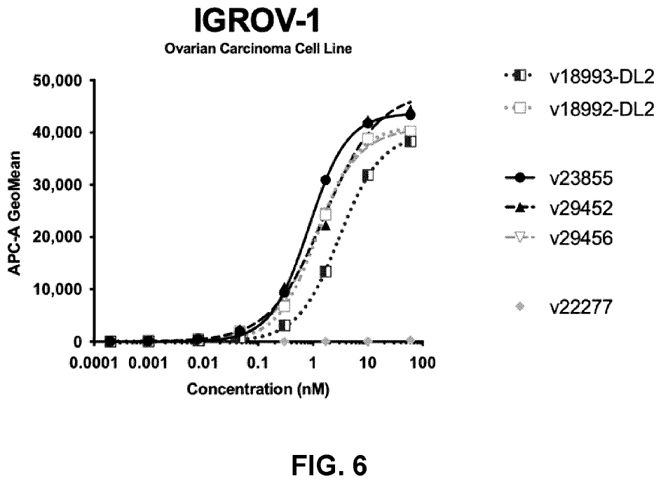

Results for parental chimeric construct (v23855) and all humanized antibody variants are tabulated in Table 16.1. Full dose-response binding curves are represented for parental chimeric antibody (v23855) and two representative humanized antibody variants (v29452, v29456) in FIG. 6.

TABLE 16.1
Binding of parental chimeric and humanized
antibody variants to IGROV-1 cells

| | IGROV-1 | | |
|---|---|---|---|
| | | Kd | Curve Hill |
| Test Antibody | Bmax | (nM) | Slope (h) |
| v23855 (parental chimeric) | 43,706 | 0.84 | 1.2 |
| v18993 (lifastuzumab)-DL2 | 39,758 | 3.00 | 1.1 |
| v18992 (MX35)-DL2 | 41,234 | 1.22 | 1.2 |
| v29449 (H4L3) | 44,460 | 0.79 | 1.3 |
| v29450 (H3L3) | 43,809 | 0.96 | 1.2 |
| v29451 (H2L3) | 46,170 | 1.49 | 1.1 |
| v29452 (H1L3) | 47,763 | 1.60 | 0.9 |
| v29453 (H4L2) | 44,480 | 0.82 | 1.2 |
| v29454 (H3L2) | 42,278 | 0.92 | 1.0 |
| v29455 (H2L2) | 41,917 | 1.18 | 1.1 |
| v29456 (H1L2) | 40,867 | 1.10 | 1.1 |
| v29457 (H4L1) | 44,539 | 1.73 | 1.0 |
| v29458 (H3L1) | 40,793 | 1.39 | 1.1 |
| v29459 (H2L1) | 40,941 | 1.61 | 1.2 |
| v29460 (H1L1) | 40,071 | 1.67 | 1.2 |
| v22277 (Palivizumab) | NB | NB | NB |

All humanized antibody variants bound to IGROV-1 cells similarly, yielding apparent Kd values within 2-fold and comparable Bmax values. Reference antibody MX35-DL2 ADC showed comparable binding to chimeric v23855 and humanized antibodies. Humanized antibody lifastuzumab-DL2 ADC showed lower binding compared to all other targeted antibodies, with a lower Bmax value and greater apparent Kd value. Negative control palivizumab (v22277) showed no cellular binding (NB), as expected.

### Example 17: Preparation of Antibody-Drug Conjugates

Antibody-drug conjugates shown in Table 17.1 below were prepared. Exemplary protocols are provided below.

TABLE 17.1
Antibody-Drug Conjugates

| Variant | Antibody | Drug-Linker¹ | DAR |
|---|---|---|---|
| v29456 | Humanized | MC-GGFG-AM-DXd1 | 8 |
| | | MC-GGFG-AM-Compound 139 | 4 |
| | | MC-GGFG-AM-Compound 139 | 8 |
| | | MC-GGFG-Compound 141 | 4 |
| | | MC-GGFG-Compound 141 | 8 |
| | | MT-GGFG-AM-Compound 139 | 8 |
| | | MT-GGFG-AM-Compound 141 | 8 |
| | | MT-GGFG-AM-Compound 136 | 8 |
| | | MT-GGFG-AM-Compound 129 | 8 |
| | | MT-GGFG-Compound 141 | 8 |
| | | MT-GGFG-Compound 140 | 8 |
| | | MT-GGFG-Compound 148 | 8 |

Controls

| | | | |
|---|---|---|---|
| v22277 | Humanized | MC-GGFG-AM-DXd1 | 8 |
| (palivizumab) | | | |
| v21995 | — | MC-GGFG-AM-DXd1 | 8 |
| (palivizumab) | | MC-GGFG-Compound 141 | 8 |
| | | MT-GGFG-Compound 140 | 8 |
| | | MT-GGFG-AM-Compound 141 | 8 |

v29456-MC-GGFG-AM-DXd1 DAR8: A solution (2.47 mL) of the humanized variant v29456 (54 mg) in PBS, pH 7.4 was diluted in PBS, pH 7.4 (1.00 mL) and reduced by addition of 5 mM diethylenetriamine pentaacetic acid (DTPA) (0.90 mL in PBS, pH adjusted to 7.4) and 25 mM of an aqueous tris(2-carboxyethyl)phosphine (TCEP) solution (134 μL, 9.0 eq.). After 4 hours at 37° C., the reduced antibody was purified by passage over a Zeba™ Spin Desalting Columns (40 KDa MWCO; Thermo Scientific™) pre-equilibrated with 1 mM DTPA in PBS, pH 7.4. An aliquot of the reduced antibody solution (13.5 mg, 1.32 mL) was diluted with 1 mM DTPA (33.5 μL in PBS, pH adjusted to 7.4). To the antibody solution was added 38.3 μL of DMSO and an excess of MC-GGFG-AM-DXd1 (111.7 μL; 12 eq.) from a 10 mM DMSO stock solution. The conjugation reaction proceeded at room temperature with mixing for 120 min. At that time, additional drug-linker (14 μL, 1.5 eq.) was added. The conjugation reaction proceeded at room temperature with mixing for an additional 60 min. An excess of a 10 mM N-acetyl-L-cysteine solution (76.8 μL, 8 eq.) was added to quench the conjugation reaction.

v29456-MC-GGFG-AM-Compound 139 DAR8, v29456-MC-GGFG-Compound 141 DAR8. A solution (1.14 mL) of humanized variant v29456 (25 mg) in PBS, pH 7.4 was diluted in PBS, pH 7.4 (252 μL) and reduced by addition of 5 mM diethylenetriamine pentaacetic acid (DTPA) (0.4 mL in PBS, pH adjusted to 7.4) and 10 mM of an aqueous tris(2-carboxyethyl)phosphine (TCEP) solution (207 μL, 12 eq.). After 180 minutes at 37° C., the reduced antibody was purified by passage over a Zeba™ Spin Desalting Columns (40 KDa MWCO; Thermo Scientific™) pre-equilibrated with 10 mM NaOAc pH 5.5. To the antibody solution was added 667 μL of 10 mM NaOAc, pH 5.5, 197 μL of DMSO and an excess of either MC-GGFG-AM-Compound 139 or MC-GGFG-Compound 141 (243 μL; 16 eq.) from a 10 mM DMSO stock solution. The conjugation reaction proceeded at room temperature with mixing for 120 minutes. An excess of 10 mM N-acetyl-L-cysteine solution (971 μL, 64 eq.) was added to quench each conjugation reaction.

v29456-MC-GGFG-AM-Compound 139 DAR4, v29456-MC-GGFG-Compound 141 DAR4. A solution (2.01 mL) of humanized variant v29456 (44 mg) in PBS, pH 7.4 was diluted in PBS, pH 7.4 (734 μL) and reduced by addition of 5 mM diethylenetriamine pentaacetic acid (DTPA) (704 μL in PBS, pH adjusted to 7.4) and 10 mM of an aqueous tris(2-carboxyethyl)phosphine (TCEP) solution (72.8 μL, 2.4 eq.). After 120 minutes at 37° C., 1.76 mL of the reduced antibody was diluted with 1.76 mL of PBS, pH 7.4 and 0.44 mL of 100 mM NaOAc, pH 5.5. To the antibody solution was added 288 μL of DMSO and an excess of either MC-GGFG-AM-Compound 139 or MC-GGFG-Compound 141 (152 μL; 10 eq.) from a 10 mM DMSO stock solution. The conjugation reaction proceeded at room temperature with mixing for 120 minutes. An excess of 10 mM N-acetyl-L-cysteine solution (607 μL, 40 eq.) was added to quench each conjugation reaction.

v29456-MT-GGFG-AM-Compound 139, v29456-MT-GGFG-AM-Compound 141 DAR8: A solution (2.47 mL) of the humanized variant v29456 (54 mg) in PBS, pH 7.4 was diluted in PBS, pH 7.4 (1.00 mL) and reduced by addition of 5 mM diethylenetriamine pentaacetic acid (DTPA) (0.90 mL in PBS, pH adjusted to 7.4) and 25 mM of an aqueous tris(2-carboxyethyl)phosphine (TCEP) solution (134 μL, 9.0 eq.). After 4 hours at 37° C., the reduced antibody was purified by passage over a Zeba™ Spin Desalting Columns (40 KDa MWCO; Thermo Scientific™) pre-equilibrated with 1 mM DTPA in PBS, pH 7.4. An aliquot of the reduced antibody solution (13.5 mg, 1.32 mL) was diluted with 1 mM DTPA (33.5 μL in PBS, pH adjusted to 7.4). To the antibody solution was added 38.3 μL of DMSO and an excess of either MT-GGFG-AM-Compound 139, or MT-GGFG-AM-Compound 141 (111.7 μL; 12 eq.) from a 10 mM DMSO stock solution. The conjugation reaction proceeded at room temperature with mixing for 180 min. An excess of a 10 mM N-acetyl-L-cysteine solution (55.8 μL, 6 eq.) was added to quench the conjugation reaction.

v29456-MT-GGFG-AM-Compound 136, v29456-MT-GGFG-AM-Compound 129 DAR8 A solution (456 μL) of the humanized variant v29456 (10 mg) in PBS, pH 7.4 was diluted in PBS, pH 7.4 (1.06 mL) and reduced by addition of 5 mM diethylenetriamine pentaacetic acid (DTPA) (0.40 mL in PBS, pH adjusted to 7.4) and 10 mM of an aqueous tris(2-carboxyethyl)phosphine (TCEP) solution (82.7 μL, 12.0 eq.). After 3 hours at 37° C., the reduced antibody was purified by passage over a Zeba™ Spin Desalting Columns (40 KDa MWCO; Thermo Scientific™) pre-equilibrated with 10 mM NaOAc, pH 4.5. To an aliquot of the reduced antibody solution (1 mg, 200 μL) was added 20 μL of DMSO and an excess of MT-GGFG-AM-Compound 136 or MT-GGFG-AM-Compound 129 (13.8 μL; 20 eq.) from a 10 mM DMSO stock solution. The conjugation reaction proceeded at room temperature with mixing for 2.5 h.

v29456-MT-GGFG-Compound 141 DAR8: A solution (2.78 mL) of the humanized variant v29456 (61 mg) in PBS, pH 7.4 was diluted in PBS, pH 7.4 (3.92 mL) and reduced by addition of 5 mM diethylenetriamine pentaacetic acid (DTPA) (1.80 mL in PBS, pH adjusted to 7.4) and 10 mM of an aqueous tris(2-carboxyethyl)phosphine (TCEP) solution (505 μL, 12 eq.). After 3 hours at 37° C., the reduced antibody was purified by passage over a Zeba™ Spin Desalting Columns (7 KDa MWCO; Thermo Scientific™) pre-equilibrated with 10 mM NaOAc, pH 4.5. To an aliquot of the reduced antibody solution (20 mg, 2.95 mL) was added 295 μL of DMSO and an excess of MT-GGFG-Compound 141 (206.8 μL; 15 eq.) from a 10 mM DMSO stock solution. The conjugation reaction proceeded at room temperature with mixing for 2.5 h. An excess of a 10 mM N-acetyl-L-cysteine solution (165.5 μL, 12 eq.) was added to quench the conjugation reaction.

v29456-MT-GGFG-Compound 140 DAR8: A solution (1.51 mL) of the humanized variant v29456 (33 mg) in PBS, pH 7.4 was diluted in PBS, pH 7.4 (0.61 mL) and reduced by addition of 5 mM diethylenetriamine pentaacetic acid (DTPA) (0.55 mL in PBS, pH adjusted to 7.4) and 25 mM of an aqueous tris(2-carboxyethyl)phosphine (TCEP) solution (81.9 μL, 9.0 eq.). After 3.5 hours at 37° C., the reduced antibody was purified by passage over a Zeba™ Spin Desalting Columns (40 KDa MWCO; Thermo Scientific™) pre-equilibrated with 1 mM DTPA in PBS, pH 7.4. To an aliquot of the reduced antibody solution (10 mg, 1.55 mL) was added 48.5 μL of DMSO and an excess of either MT-GGFG-Compound 140 (124.1 μL; 11 eq.) from a 10 mM DMSO stock solution. Additional drug-linker (73.1 μL; 7 eq) was added. The conjugation reaction proceeded at room temperature with mixing for 3.5 h. An excess of a 10 mM N-acetyl-L-cysteine solution (180.9 μL, 16.5 eq.) was added to quench the conjugation reaction.

v29456-MT-GGFG-Compound 148 DAR8: A solution (1.51 mL) of the humanized variant v29456 (33 mg) in PBS, pH 7.4 was diluted in PBS, pH 7.4 (0.61 mL) and reduced by addition of 5 mM diethylenetriamine pentaacetic acid (DTPA) (0.55 mL in PBS, pH adjusted to 7.4) and 25 mM of an aqueous tris(2-carboxyethyl)phosphine (TCEP) solution (81.9 μL, 9.0 eq.). After 3.5 hours at 37° C., the reduced antibody was purified by passage over a Zeba™ Spin Desalting Columns (40 KDa MWCO; Thermo Scientific™) pre-equilibrated with 1 mM DTPA in PBS, pH 7.4. To an aliquot of the reduced antibody solution (10 mg, 1.48 mL) was added 164.7 μL of DMSO and an excess of MT-GGFG-Compound 148 (82.7 μL; 9 eq.) from a 10 mM DMSO stock solution. Additional drug-linker (31.7 μL; 3 eq.) was added. The conjugation reaction proceeded at room temperature with mixing for 3.5 h. An excess of a 10 mM N-acetyl-L-cysteine solution (57.1 μL, 6 eq.) was added to quench the conjugation reaction.

v22277-MC-GGFG-AMI-DXd1 DAR8: A solution (2.18 mL) of the control variant v22277 (10 mg) in PBS, pH 7.4 was diluted in PBS, pH 7.4 (4.1 μL) and reduced by addition of 5 mM diethylenetriamine pentaacetic acid (DTPA) (563 μL in PBS, pH adjusted to 6.7) and 10 mM of an aqueous tris(2-carboxyethyl)phosphine (TCEP) solution (68.9 μL, 10.0 eq.). After 3 hours at 37° C., the reduced antibody was purified by passage over a Zeba™ Spin Desalting Columns (40 KDa MWCO; Thermo Scientific™) pre-equilibrated with 10 mM NaOAc, pH 5.5. To the reduced antibody solution was added 230.0 μL of DMSO and an excess of MC-GGFG-AM-DXd1 (51.7 μL; 15 eq.) from a 20 mM DMSO stock solution. The conjugation reaction proceeded at room temperature with mixing for 60 min. An excess of a 20 mM N-acetyl-L-cysteine solution (51.7 μL, 15 eq.) was added to quench the conjugation reaction. The quenching reaction proceeded at 0-4° C. for 30 min.

v21995-MC-GGFG-AM-DXd, v21995-MC-GGFG-Compound 141 DAR8: A solution (1.76 mL) of the humanized variant v21995 (32 mg) in PBS, pH 7.4 was diluted in PBS, pH 7.4 (2.77 mL) and reduced by addition of 5 mM diethylenetriamine pentaacetic acid (DTPA) (1.20 mL in PBS, pH adjusted to 7.4) and 10 mM of an aqueous tris(2-carboxyethyl)phosphine (TCEP) solution (264 μL, 12.0 eq.). After 3 hours at 37° C., the reduced antibody was purified by passage over a Zeba™ Spin Desalting Columns (40 KDa MWCO; Thermo Scientific™) pre-equilibrated with 10 mM NaOAc, pH 5.5. To the reduced antibody solution was added 600 μL of DMSO and an excess of either MC-GGFG-AM-DXd or MC-GGFG-Compound 141 (308.5 μL; 14 eq.) from a 10 mM DMSO stock solution. The conjugation reaction proceeded at room temperature with mixing for 90 min. An excess of a 10 mM N-acetyl-L-cysteine solution (198 μL, 9 eq.) was added to quench the conjugation reaction. The quenching reaction proceeded at 0-4° C. for 30 min.

v21995-MT-GGFG-Compound 140. MT-GGFG-AM-Compound 141 DAR8: A solution (2.07 mL) of the humanized variant v21995 (30 mg) in PBS, pH 7.4 was diluted in PBS, pH 7.4 (2.48 mL) and reduced by addition of 5 mM diethylenetriamine pentaacetic acid (DTPA) (1.20 mL in PBS, pH adjusted to 7.4) and 10 mM of an aqueous tris(2-carboxyethyl)phosphine (TCEP) solution (248 μL, 12.0 eq.). After 3 hours at 37° C., the reduced antibody was purified by passage over a Zeba™ Spin Desalting Columns (40 KDa MWCO; Thermo Scientific™) pre-equilibrated with pH 5.5PBS, pH 7.4. To an aliquot of the reduced antibody solution (3.00 mL, 15 mg) was added 150 μL of DMSO and an excess of either MT-GGFG-Compound 140 or MT-GGFG-AM-Compound 141 (77.5 μL; 7.5 eq.) from a 10 mM DMSO stock solution. The conjugation reaction proceeded at room temperature with mixing for 30 min. 150 μL of DMSO and an excess of either MT-GGFG-Compound 140 or MT-GGFG-AM-Compound 141 (77.5 μL; 7.5 eq.) from a 10 mM DMSO stock solution were added again, and the conjugation reaction proceeded at room temperature with mixing for another 30 min.

### Example 18: Purification and Characterization of Antibody-Drug Conjugates

ADCs prepared as described in Example 17 were purified on an AKTA™ pure chromatography system (Cytiva Life Sciences, Marlborough, MA) using a 53 mL HiPrep 26/10 Desalting column (Cytiva Life Sciences, Marlborough, MA) and a mobile phase consisting of 10 mM NaOAc, pH 4.5 with 150 mM NaCl and a flow rate of 10 mL/min. The purified ADC was then sterile filtered (0.2 μm).

Alternatively, ADCs prepared as described in Example 17 were purified by two to three passages over a Zeba™ Spin Desalting Columns (40 KDa MWCO; Thermo Scientific™) pre-equilibrated with PBS, pH 7.4 for the first passage, and with 10 mM NaOAC, pH 4.5 or pH 5.5 for the subsequent passages. The purified ADC was then sterile filtered (0.2 μm).

Following purification, the concentration of the ADCs was determined by a BCA assay with reference to a standard curve generated using the humanized variant v29456. Alternatively, concentrations were estimated by measurement of absorption at 280 nm using extinction coefficients taken from the literature (European Patent No. 3 342 785, for MC-GGFG-AM-DXd1) or determined experimentally (for the remaining drug-linkers). ADCs were also characterized by hydrophobic interaction chromatography (HIC) and size exclusion chromatography (SEC) as described below.

### Hydrophobic Interaction Chromatography

Antibody and ADCs were analyzed by HIC to estimate the drug-to-antibody ratio (DAR). Chromatography was performed on an Agilent Infinity II 1290 HPLC (Agilent Technologies, Santa Clara, CA) using a TSKgel® Butyl-NPR column (2.5 μm, 4.6×35 mm; TOSOH Bioscience GmbH, Griesheim, Germany) and employing a gradient of 95/5% MPA/MPB to 5/95% MPA/MPB over a period of 12 minutes at a flow rate of 0.5 mL/min (MPA=1.5 M (NH₄)₂SO₄, 25 mM NaₓPO₄, pH 7 and MPB=75% 25 mM NaₓPO₄, pH 7, 25% isopropanol). Detection was by absorbance at 280 nm.

### Size Exclusion Chromatography

The extent of aggregation of the antibody and ADCs (~15-150 μg, 5 μL injection volume) was assessed by SEC on an Agilent Infinity 111260 HPLC (Agilent Technologies, Santa Clara, CA) using an AdvanceBio SEC column (300 angstroms, 2.7 μm, 7.8×150 mm) (Agilent, Santa Clara, California) and a mobile phase consisting of 150 mM phosphate, pH 6.95 and a flow rate of 1 mL/min. Detection was by absorbance at 280 nm.

### Results

The individual contributions of the DAR0, DAR2, DAR4, DAR6 and DAR8 species to the average DAR of the purified ADCs were assessed by integration of the HPLC-HIC chromatogram. The average drug to antibody ratio (DAR) of each ADC was determined by the weighted average of each DAR species. The average DAR for each ADC, when rounded to the nearest integer, was the same as the target DAR shown in Table 18.1.

The extent of aggregation and monomer content was assessed by integration of the HPLC-SEC chromatogram. The monomer peak of each ADC was identified as the peak with the same retention time as the unconjugated antibody from which each ADC was derived from. All peaks with an earlier retention time relative to the monomer species was determined to be aggregated species. Percent monomer species determined for each ADC is shown in Table 18.1. All ADC preparations showed >95% monomer species.

TABLE 18.1
Characterization of ADCs

| | | Target | % |
|---|---|---|---|
| Variant | Drug-Linker¹ | DAR | Monomer |
| v29456 | MC-GGFG-AM-DXd1^{a} | 8 | 98.5 |
| | MC-GGFG-AM-Compound 139^{b} | 4 | 100 |
| | MC-GGFG-AM-Compound 139^{b} | 8 | 100 |
| | MC-GGFG-Compound 141^{b} | 4 | 99.3 |
| | MC-GGFG-Compound 141^{b} | 8 | 98.8 |
| | MT-GGFG-AM-Compound 139^{a} | 8 | 98.8 |
| | MT-GGFG-AM-Compound 141^{a} | 8 | 100 |
| | MT-GGFG-AM-Compound 136^{a} | 8 | 100 |
| | MT-GGFG-AM-Compound 129^{a} | 8 | 100 |
| | MT-GGFG-Compound 141^{a} | 8 | 99.7 |
| | MT-GGFG-Compound 140^{a} | 8 | 98.8 |
| | MT-GGFG-Compound 148^{a} | 8 | 100 |
| v22277 | MC-GGFG-AM-DXd1^{a} | 8 | 98.5 |
| v21995 | MC-GGFG-AM-DXd1^{b} | 8 | 100 |
| | MC-GGFG-Compound 141^{b} | 8 | 97.2 |
| | MT-GGFG-Compound 140^{a} | 8 | 98.8 |
| | MT-GGFG-AM-Compound 141^{a} | 8 | 98.6 |

^{a}Purified using Zeba ™ Spin Desalting Columns
^{b}Purified using AKTA ™ pure chromatography system

### Example 19: Bystander Activity of Anti-NaPi2B ADCs

The ability of v29456 ADCs to exert a bystander killing effect on cancer cells was assessed according to the method described below. Bystander killing can occur after target-specific uptake of an ADC into an antigen-positive cell. In this case, catabolism of the ADC results in release of payload or an active catabolite, which then crosses the cell membrane of nearby cells to elicit their death.

The ADCs tested were v29456-MT-GGFG-AM-Compound 139, v29456-MT-GGFG-AM-Compound 141, v29456-MT-GGFG-Compound 141, v29456-MT-GGFG-Compound 140, v29456-MT-GGFG-Compound 148. Positive controls v29456-MC-GGFG-AM-DXd1 and v29456-MCvcPABC-MMAE, whose drug linkers have been known to be active in bystander activity, and negative (non-NaPi2b targeting) controls palivizumab (v22277) MC-GGFG-AM-DXd and palivizumab (v22277) MCvcPABC-MMAE, were included. Cell lines used were HCC-78 (high NaPi2b expression) and EBC-1 (negative NaPi2b expression).

NaPi2b-positive HCC-78 and NaPi2b-negative EBC-1 cells were seeded either as mono-cultures or co-cultures in a 48-well plate at 15,000 cells and 5,000 cells, respectively, in 100 μL assay media (RPMI1640+10% FBS). ADCs were diluted to 10 nM in assay media and 100 μL was added to the cell-containing plates (5 nM final ADC concentration). Cells were incubated with ADCs for 4 days under standard culturing conditions (37° C./5% CO₂). Following incubation, cells were dissociated, washed, and stained using a viability dye, YO-PRO®-1 (ThermoFisher Scientific, Waltham, MA), and an anti-NaPi2b antibody, MX35, conjugated to Alexa Fluor® 647, for 20 minutes at 4° C. After incubation, cells were washed in FACS buffer, resuspended in 70 μL FACS buffer per well, and 35 μL per well were analyzed on a BD LSRFortessa™ Cell Analyzer (BD Biosciences, Franklin Lake, NJ). Dead cells were excluded by gating on YO-PRO®-1 staining. The number of HCC-78 and EBC-1 cells were determined by the number of events in the Alexa Fluor® 647 positive (NaPi2b-positive) and Alexa Fluor® 647 negative (NaPi2b-negative) gates, respectively. Percent viability was calculated as the number of cells in treatment condition divided by the number of cells in the no-treatment condition.

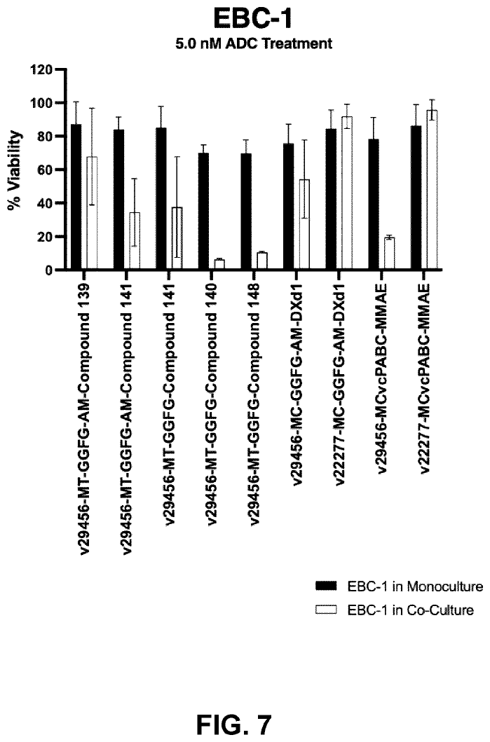

The results are provided in Table 19.1 below and in FIG. 7. Bystander effect was calculated by comparing the viability of NaPi2b-negative EBC-1 cells treated as mono-culture (black bars) with that of the cells treated as a co-culture with NaPi2b-positive HCC-78 cells (grey bars). A greater decrease in viability in co-culture compared with mono-culture indicated a greater bystander effect. NaPi2b-targeted camptothecin analogue ADCs all showed bystander capability to varying degrees. Positive controls v29456-MC-GGFG-AM-DXd1 and v29456-MCvcPABC-MMAE showed positive bystander activity, as expected. Negative controls palivizumab MC-GGFG-AM-DXd1 and palivizumab MCvcPABC-MMAE showed no cell killing in EBC-1 in mono-culture or in co-culture, indicating negative bystander activity, as expected.

TABLE 19.1
Bystander Activity of anti-NaPi2b ADCs against NaPi2b-negative
EBC-1 cell line, 5 nM ADC Treatment

| | | Average | Average | |
|---|---|---|---|---|
| | | EBC-1 % | EBC-1 % | % |
| | | viability in | viability | By- |
| | | mono- | in co- | stander |
| ADC | DAR | culture* | culture* | Activity |
| v29456-MT-GGFG-AM- | 8 | 87.1 | 67.9 | 19.2 |
| Compound 139 | | | | |
| v29456-MT-GGFG-Compound | 8 | 84.0 | 34.5 | 49.4 |
| 141 | | | | |
| v29456-MT-GGFG-AM- | 9 | 85.3 | 37.6 | 47.6 |
| Compound 141 | | | | |
| v29456-MT-GGFG-Compound | 8 | 70.0 | 6.4 | 63.7 |
| 140 | | | | |
| v29456-MT-GGFG-Compound | 8 | 69.8 | 10.6 | 59.2 |
| 148 | | | | |
| v29456-MC-GGFG-AM-DXd1 | 8 | 75.8 | 54.4 | 21.4 |
| v22277-MC-GGFG-AM-DXd1 | 8 | 84.6 | 91.9 | −7.3 |
| v29456-MCvcPABC-MMAE | 8 | 78.4 | 19.7 | 58.7 |
| v22277-MCvcPABC-MMAE | 4 | 86.4 | 95.8 | −9.4 |

*Relative to untreated blank

### Example 20: In Vitro Cytotoxicity of Antibody-Drug Conjugates -2D Monolayer

The cell growth inhibition (cytotoxicity) capabilities of the humanized variant v29456 conjugated to various drug-linkers, conjugated at an antibody-to-drug ratio of 8.0, were assessed in a panel of NaPi2b-expressing cell lines as described below. Cell lines used were HCC-78 (lung carcinoma), IGROV-1 (ovarian adenocarcinoma), and HCT116 (colorectal carcinoma; NaPi2b-negative). ADCs with antibody palivizumab (anti-RSV) (v22277) were used as non-targeted controls.

Briefly, cells were seeded in 384-well plates and treated with a titration of test article prepared in cell growth medium. Cells were incubated for 4 days under standard culturing conditions. After incubation, CellTiter-Glo® reagent (Promega Corporation, Madison, WI) was spiked in all wells and luminescence corresponding to ATP present in each well was measured using a Synergy™H1 plate reader (BioTek Instruments, Winooski, VT). Percent cytotoxicity values were calculated based on blank wells (no test article added), and plotted against test article concentration using GraphPad Prism 9 software (GraphPad Software, San Diego, CA). EC50 values were calculated based on a non-linear regression log(agonist) versus response, variable slope (four parameters) by GraphPad Prism 9.

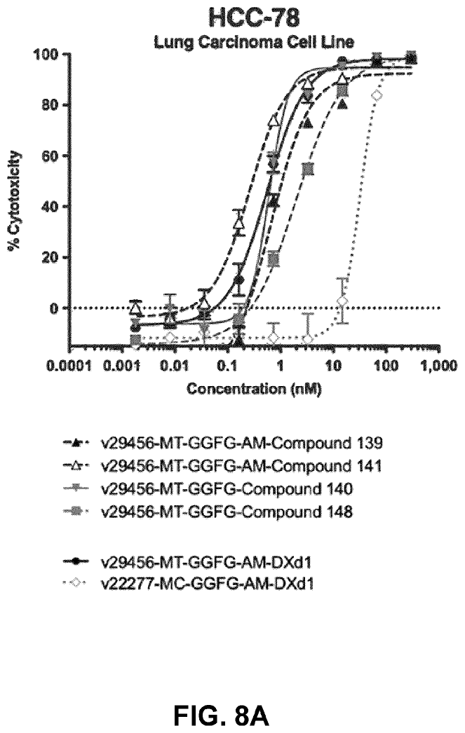

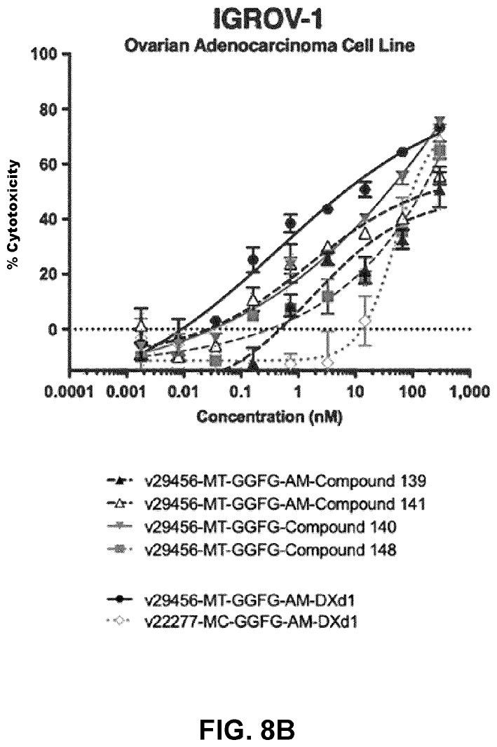

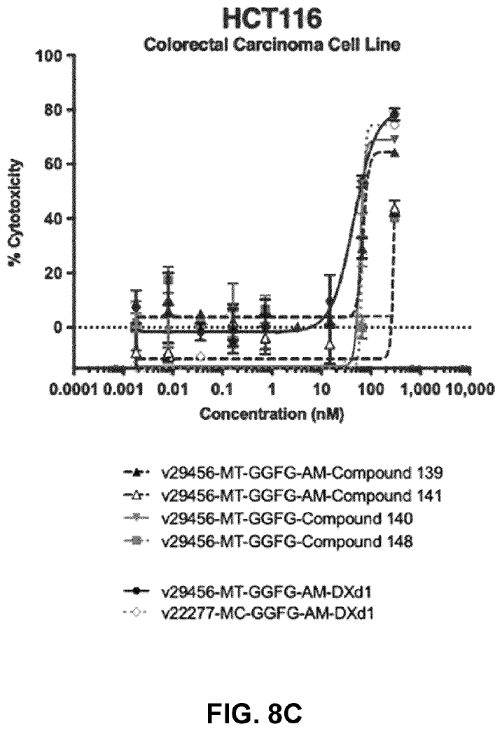

The results are shown in Table 20.1 and representative curves are plotted in FIG. 8A (HCC-78), FIG. 8B (IGROV-1), and FIG. 8C (HCT116).

TABLE 20.1
In vitro Cytotoxicity-2D Monolayer-DAR 8 Camptothecin Analogues

| | | EC50 (nM) | | |
|---|---|---|---|---|
| ADC | DAR | HCC-78 | IGROV-1 | HCT116 |
| v29456-MT-GGFG-AM- | 8.0 | 0.43 | 4.48 | 55.82 |
| Compound 136 | | | | |
| v29456-MT-GGFG-AM- | 8.0 | 0.65 | 1.71 | 69.47 |
| Compound 139 | | | | |
| v29456-MT-GGFG- | 8.0 | 0.60 | IC* | 59.53 |
| Compound 140 | | | | |
| v29456-MT-GGFG-AM- | 8.0 | 0.42 | 1.11 | 9.24 |
| Compound 129 | | | | |
| v29456-MT-GGFG-AM- | 8.0 | 0.26 | 1.56 | IC |
| Compound 141 | | | | |
| v29456-MT-GGFG- | 8.0 | 1.98 | IC* | >300 |
| Compound 148 | | | | |
| v29456-MT-GGFG-AM- | 8.0 | 0.55 | 0.45 | 44.76 |
| DXd1 | | | | |
| v22277-MC-GGFG-AM- | 8.0 | 31.83 | 50.91 | 67.05 |
| DXd1 | | | | |

*Incomplete curve

All v29456 ADCs displayed significant cytotoxicity in NaPi2b expressing cell lines HCC-78 and IGROV-1, yielding single-digit nanomolar or lower EC50 values after 4-day treatment. In NaPi2b-negative cell line HCT116, v29456 and palivizumab ADCs yielded comparable potency, indicating NaPi2b-targeted ADCs did not show target-dependent cytotoxicity, as expected.

### Example 21: In Vitro Cytotoxicity of Antibody-Drug Conjugates -2D Monolayer

The cell growth inhibition (cytotoxicity) capabilities of the humanized variant v29456 conjugated to select camptothecin drug-linkers conjugated at an antibody-to-drug ratio of 8 and 4 were assessed against NaPi2b-expressing cell lines IGROV-1 (ovarian adenocarcinoma) and TOV-21G (ovarian adenocarcinoma), according to the methods described in Example 20. ADCs with antibody palivizumab (anti-RSV) (v21995) were used as non-targeted controls.

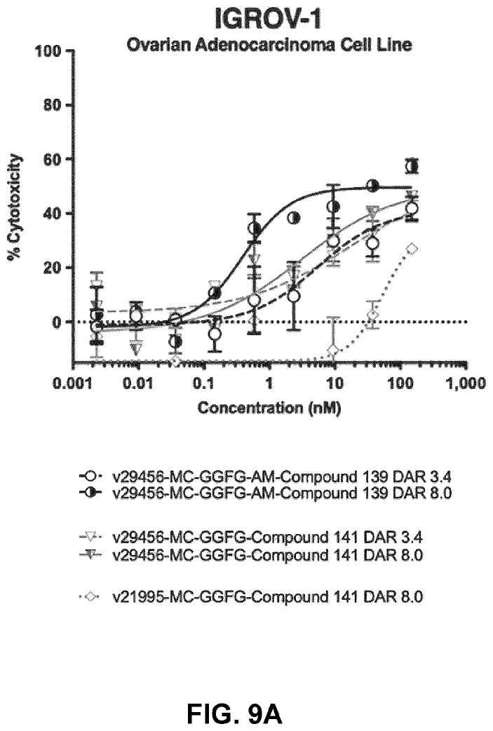

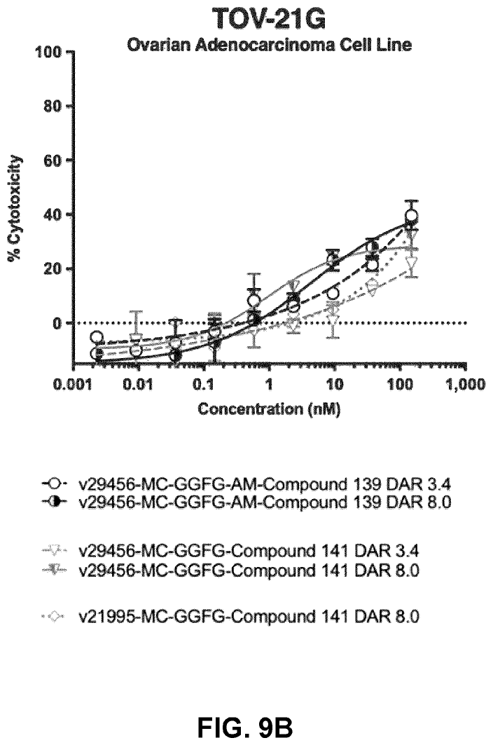

The results are shown in Table 21.1 and representative curves are plotted in FIG. 9A (IGROV-1) and FIG. 9B (TOV-21G).

TABLE 21.1
In vitro Cytotoxicity-2D Monolayer-Comparison of
DAR 4 and DAR 8 ADCs

| | | EC50 (nM) | |
|---|---|---|---|
| Sample | DAR | IGROV-1 | TOV-21G |
| v29456-MC-GGFG-AM-Compound | 3.4 | 5.46 | IC* |
| 139 | | | |
| v29456-MC-GGFG-AM-Compound | 8.0 | 0.39 | 4.30 |
| 139 | | | |
| v29456-MC-GGFG-Compound 141 | 3.4 | 16.45 | IC* |
| v29456-MC-GGFG-Compound 141 | 8.0 | 3.01 | 0.93 |
| v21995-MC-GGFG-Compound 141 | 8.0 | 53.35 | IC* |

*Incomplete curve

Humanized variant v29456 ADCs displayed targeted cytotoxicity against high NaPi2b-expressing cell line IGROV-1 and moderate NaPi2b-expressing cell line TOV-21G, with higher potency against IGROV-1 than TOV-21G, thereby demonstrating NaP2ib-dependent killing. ADCs with a DAR of 8 showed greater potency than ADCs with a DAR of 3.4. As expected, palivizumab ADC control showed less activity against both cell lines compared to NaPi2b-targeted ADCs.

### Example 22: In Vitro Cytotoxicity of DAR 8 Camptothecin Analogue Antibody-Drug Conjugates—3D Spheroids

The 3D cytotoxicity capabilities of the humanized variant v29456 conjugated to various drug linkers were assessed in a panel of NaPi2b-expressing cell line spheroids as described below. Cell lines used were HCC-78 (lung carcinoma) and IGROV-1 (ovarian adenocarcinoma). ADCs with antibody palivizumab (anti-RSV) (v22277, v21995) were used as non-targeted controls.

Briefly, cells were seeded in Ultra-Low Attachment 384-well plates at 3,000 cells/well, centrifuged, and incubated for 3 days under standard culturing conditions to allow for spheroid formation and growth. Monoculture cell line spheroids were then treated with a titration of test article, generated in cell growth medium. Spheroids were incubated for 6 days under standard culturing conditions. After incubation, CellTiter-Glo® 3D reagent (Promega Corporation, Madison, WI) was spiked in all wells. Plates were incubated in the dark at room temperature for 1 hour and luminescence was quantified using a BioTek Cytation 5 Cell Imaging Multi-Mode Reader (Agilent Technologies, Inc., Santa Clara, CA). Percent cytotoxicity values were calculated based on blank wells (no test article added), and plotted against test article concentration using GraphPad Prism 9 software (GraphPad Software, San Diego, CA). EC50 values were calculated based on a non-linear regression log(agonist) versus response, variable slope (four parameters) by GraphPad Prism 9.

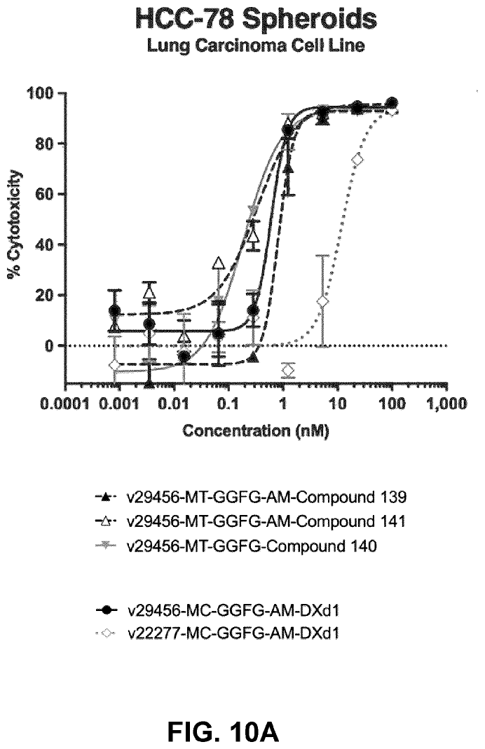

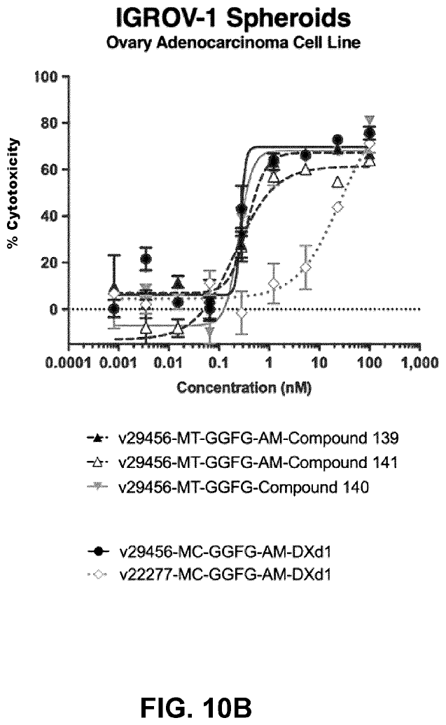

The results are shown in Table 22.1 and representative curves are plotted in FIG. 10A (HCC-78) and FIG. 10B (IGROV-1).

TABLE 22.1
In vitro Cytotoxicity-3D Spheroids-DAR 8 Camptothecin Analogues

| | | EC50 (nM) | |
|---|---|---|---|
| | | HCC-78 | IGROV-1 |
| ADC | DAR | Spheroids | Spheroids |
| v29456-MT-GGFG-AM-Compound | 8 | 0.82 | 0.37 |
| 139 | | | |
| v29456-MT-GGFG-AM-Compound | 8 | 0.33 | 0.21 |
| 141 | | | |
| v21995-MT-GGFG-Compound 141 | 8 | 14.98 | IC* |
| v29456-MT-GGFG-Compound 140 | 8 | 0.19 | 0.25 |
| v21995-MT-GGFG-Compound 140 | 8 | 4.19 | 14.77 |
| v29456-MC-GGFG-AM-DXd1 | 8 | 0.60 | 0.27 |
| v22277-MC-GGFG-AM-DXd1 | 8 | 11.85 | 26.45 |

*Incomplete curve

All v29456 ADCs showed comparable or improved potency over v29456 conjugated to DXd1, with EC50 values at sub-nanomolar range, against both NaPi2b-expressing cell lines. As expected, palivizumab ADC controls showed less activity against both cell lines compared to NaPi2b-targeted ADCs.

### Example 23: In Vitro Cytotoxicity of DAR 4 and DAR 8 Antibody-Drug Conjugates—3D Spheroids

The cell growth inhibition (cytotoxicity) capabilities of the humanized variant v29456 conjugated to select camptothecin drug-linkers conjugated at an antibody-to-drug ratio of 8 and 4 were assessed against NaPi2b-expressing cell line spheroids according to the methods described in Example 22. Cell lines used were IGROV-1 (ovarian adenocarcinoma) in monoculture and TOV-21G (ovarian adenocarcinoma) in co-culture with HDFa (adult human dermal fibroblasts, Thermo Fisher Scientific, Waltham, MA) at a 1:1 seeding density ratio (3,000 TOV-21G cells+3,000 HDFa cells per well). ADCs with antibody palivizumab (anti-RSV) (v21995) were used as non-targeted controls.

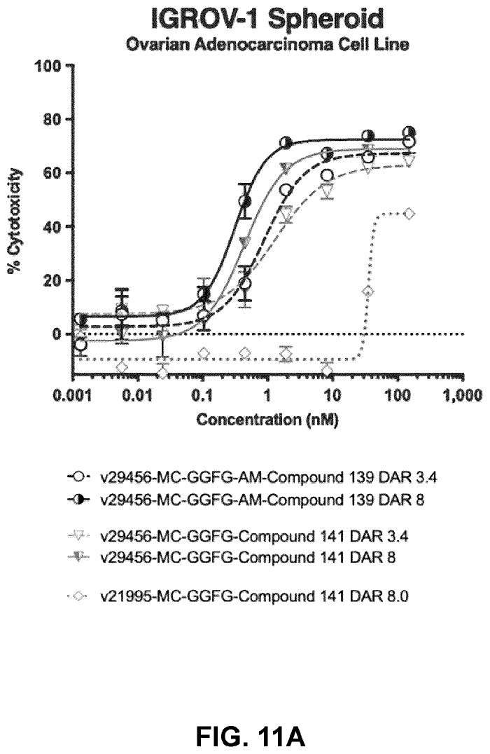

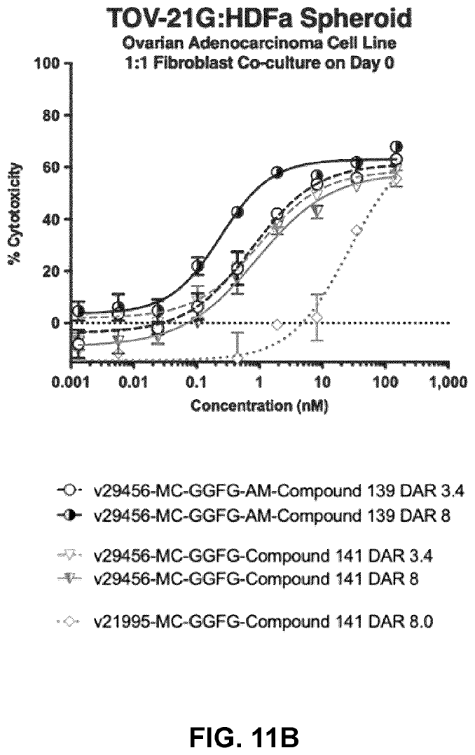

The results are shown in Table 23.1 and representative curves are plotted in FIG. 11A (IGROV-1) and FIG. 11B (TOV-21G+HDFa).

TABLE 23.1
In vitro Cytotoxicity-3D Spheroids-Comparison of
DAR 4 and DAR 8 ADCs

| | | EC50 (nM) | |
|---|---|---|---|
| | | | TOV-21G: |
| | | IGROV-1 | HDFa (1:1) |
| Sample | DAR | Spheroids | Spheroids |
| v29456-MC-GGFG-AM-Compound 139 | 3.4 | 0.88 | 0.76 |
| v29456-MC-GGFG-AM-Compound 139 | 8.0 | 0.31 | 0.24 |
| v29456-MC-GGFG-Compound 141 | 3.4 | 1.31 | 0.99 |
| v29456-MC-GGFG-Compound 141 | 8.0 | 0.44 | 0.90 |
| v21995-MC-GGFG-Compound 141 | 8.0 | 35.45 | 26.34 |

All v29456 ADCs showed targeted killing against NaPi2b-expressing spheroids, with sub-nanomolar to single-digit nanomolar EC50, while negative control palivizumab ADC showed double-digit nanomolar potency. ADCs with a DAR of 8 showed greater potency than ADCs with a DAR of 3.4.

### Example 24: In Vivo Activity of Antibody Drug Conjugates

In vivo anti-tumor activities of humanized variant v29456 ADCs were assessed in the OVCAR3 xenograft model of ovarian cancer (one study) and the NCI-H441 xenograft model of lung cancer (two studies), which both express high levels of NaPi2b. The studies were carried out as described below.

For the high NaPi2b expressing OVCAR3 ovarian cancer model, tumor fragments (~1 mm³) were implanted subcutaneously into female CB.17 SCID mice. When mean tumor volume reached ~100-150 mm³ the animals were assigned to groups, n=8 per group, and administered a single IV dose of test articles on study day 1. Tumor volume and body weight were measured twice weekly with a study duration of 60 days. Treatment groups are described in Table 24.1.

For the NCI-H441 model of lung cancer, 5×10⁶ cells in 0.1 ml 1:1 PBS:Matrigel were implanted subcutaneously into male NU-Foxn1nu mice. When mean tumor volume reached ~145 mm³ the animals were assigned to groups, n=6 per group, and administered a single IV dose of test articles on study day 0. Tumor volume and body weight were measured twice weekly with a study duration of 28-35 days. Treatment groups for NCI-H441 studies are described in Table 24.2 and Table 24.3.

For statistical analyses, a linear mixed effects model was fit to log-transformed tumor volumes, followed by F-test for the null hypothesis that mean growth rates are equal and post-hoc pairwise comparisons.

TABLE 24.1
Treatment groups for OVCAR3 Study

| Group | Treatment | DAR | Dose (mg/kg) |
|---|---|---|---|
| 1 | Vehicle | | 0 |
| 2 | v29456-MC-GGFG-AM-DXd1 | 8 | 1 |
| 3 | v29456-MC-GGFG-AM-DXd1 | 8 | 3 |
| 4 | v29456-MC-GGFG-AM-DXd1 | 8 | 10 |

TABLE 24.2
Treatment groups for NCI-H441 Study 1

| Group | Treatment | DAR | Dose (mg/kg) |
|---|---|---|---|
| 1 | Vehicle | | 0 |
| 2 | v29456-MC-GGFG-AM-DXd1 | 8 | 1 |
| 3 | v29456-MC-GGFG-AM-DXd1 | 8 | 3 |
| 4 | v29456-MC-GGFG-AM-DXd1 | 8 | 10 |

TABLE 24.3
Treatment groups for NCI-H441 Study 2

| Group | Treatment | DAR | Dose (mg/kg) |
|---|---|---|---|
| 1 | Vehicle | | 0 |
| 2 | v29456-MC-GGFG-AM-DXd1 | 8 | 0.3 |
| 3 | v29456-MC-GGFG-AM-DXd1 | 8 | 1 |
| 4 | v29456-MT-GGFG-AM-Compound 141 | 8 | 0.3 |
| 5 | v29456-MT-GGFG-AM-Compound 141 | 8 | 1 |
| 6 | v29456-MT-GGFG-AM-Compound 139 | 8 | 0.3 |
| 7 | v29456-MT-GGFG-AM-Compound 139 | 8 | 1 |
| 8 | v29456-MT-GGFG-Compound 140 | 8 | 0.3 |
| 9 | v29456-MT-GGFG-Compound 140 | 8 | 1 |
| 10 | v29456-MT-GGFG-Compound 148 | 8 | 0.3 |
| 11 | v29456-MT-GGFG-Compound 148 | 8 | 1 |
| 12 | v21995-MC-GGFG-AM-DXd1 | 8 | 1 |
| 13 | v21995-MT-GGFG-Compound 141 | 8 | 1 |
| 14 | v29456-MT-GGFG-Compound 140 | 8 | 1 |

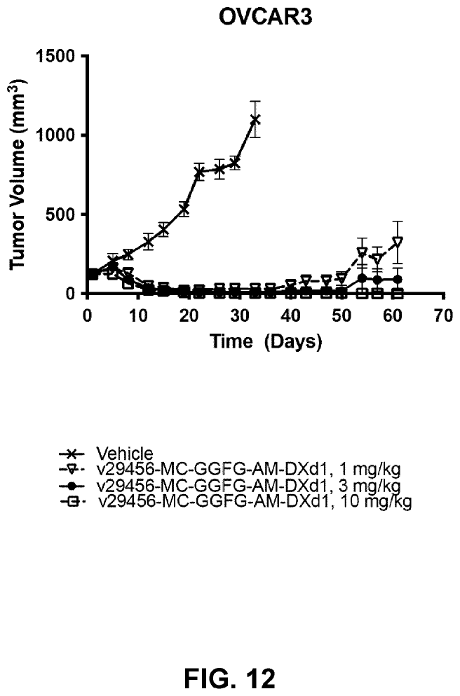

For the OVCAR3 model, the results are shown in FIG. 12, demonstrating that v29456-MC-GGFG-AM-DXd1 inhibited tumor growth at 1, 3 and 10 mg/kg.

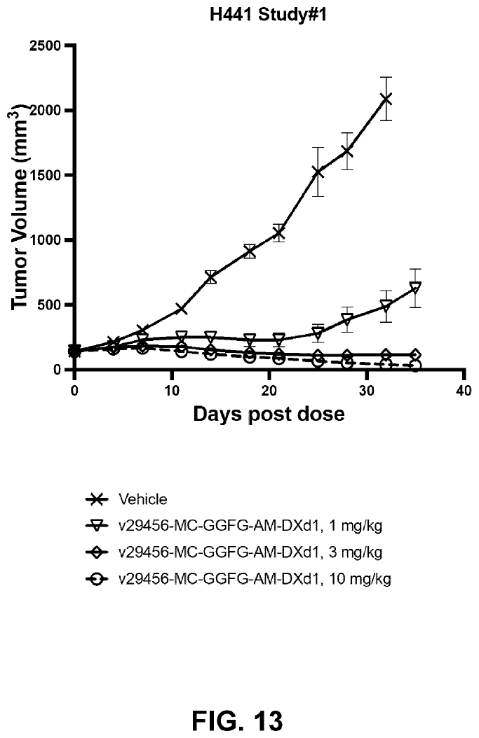

For the NCI-H441 model, Study 1, the data is provided in FIG. 13, showing that v29456-MC-GGFG-AM-DXd1 inhibited tumor growth at 1, 3 and 10 mg/kg.

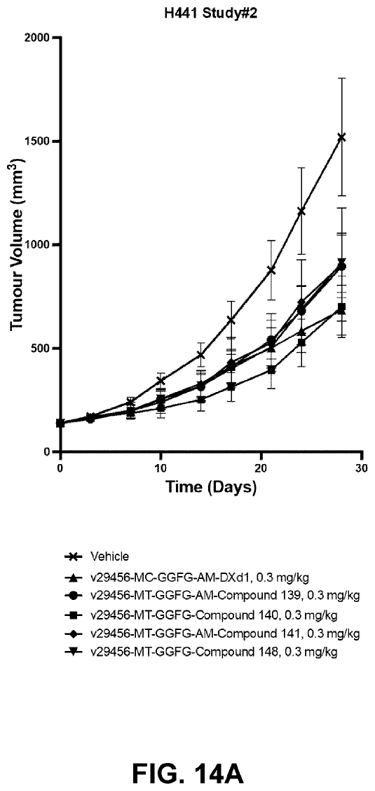

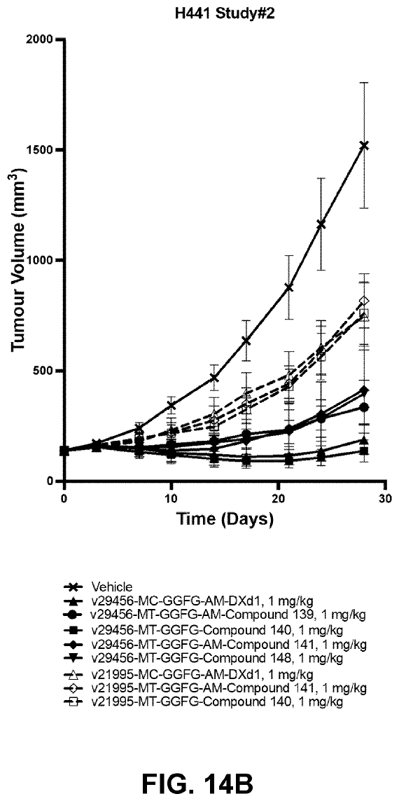

In the NCI-H441 model, Study 2, FIG. 14A depicts the data for test articles dosed at 0.3 mg/kg. FIG. 14B depicts the data for test articles dosed at 1 mg/kg. Two plots are provided for clarity; accordingly the palivizumab control ADCs shown in FIG. 14B are applicable to the plots shown on FIG. 14A. These data show that v29456-MC-GGFG-AM-DXd1, v29456-MT-GGFG-AM-Compound 139, v29456-MT-GGFG-AM-Compound 141, v29456-MT-GGFG-Compound 140, v29456-MT-GGFG-Compound 148 all demonstrated a trend of tumor growth inhibition at 0.3 mg/kg and a greater degree of tumor growth inhibition at 1 mg/kg. At 1 mg/kg, the activity of non-targeting control v21995 (palivizumab) ADCs was less than that of v29456 ADCs, demonstrating the target dependent activity of v29456 ADCs.

### Example 25: In Vivo Activity of Antibody-Drug Conjugates in Patient-Derived Models of Ovarian Cancer

In vivo anti-tumor activity of humanized variant v29456 ADCs was assessed in two patient-derived (PDX) models of ovarian cancer, CTG-2025, and CTG-0958. An ADC of reference antibody v18993 (lifastuzumab) conjugated to MCvcPABC-MMAE at DAR4 was also tested as a comparator. These studies were carried out as described below.

For the ovarian cancer PDX model CTG-2025, tumor fragments were implanted subcutaneously into female athymic Nude-Foxn1nu mice. When mean tumor volume reached ~240 mm³ the animals were assigned to groups (n=3 per group) and treated on study day 1 with a single IV dose of test articles as shown in Table 25.1.

TABLE 25.1
Treatment groups for CTG-2025 Ovarian Cancer model

| Group | Treatment | DAR | Dose (mg/kg) |
|---|---|---|---|
| 1 | Vehicle | | 0 |
| 2 | v29456-MC-GGFG-AM-Compound 139 | 8 | 6 |
| 3 | v29456-MC-GGFG-AM-Compound 139 | 4 | 6 |
| 4 | v18993 (lifastuzumab)-MCvcPABC-MMAE | 4 | 6 |

For the ovarian cancer PDX model CTG-0958, tumor fragments were implanted subcutaneously into female athymic Nude-Foxn1nu mice. When mean tumor volume reached ~225 mm³ the animals were assigned to groups (n=3 per group) and treated on study day 1 with a single IV dose of test articles as shown in Table 25.2.

TABLE 25.2
Treatment groups for CTG-0958 Ovarian Cancer Model

| Group | Treatment | DAR | Dose (mg/kg) |
|---|---|---|---|
| 1 | Vehicle | | 0 |
| 2 | v29456-MC-GGFG-AM-Compound 139 | 8 | 6 |
| 3 | v29456-MC-GGFG-AM-Compound 139 | 4 | 6 |
| 4 | v18993 (lifastuzumab)-MCvcPABC-MMAE | 4 | 6 |

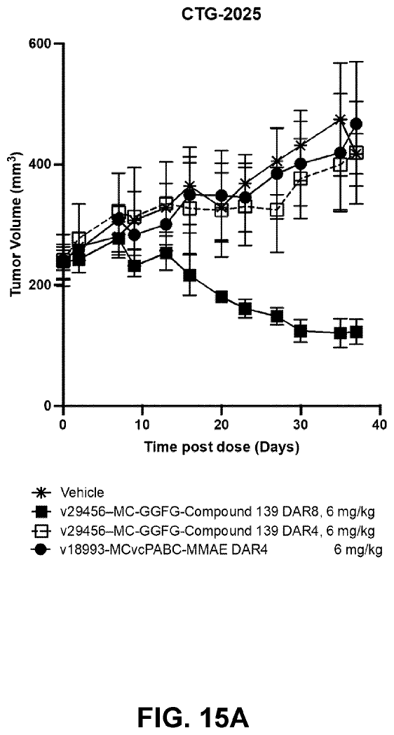

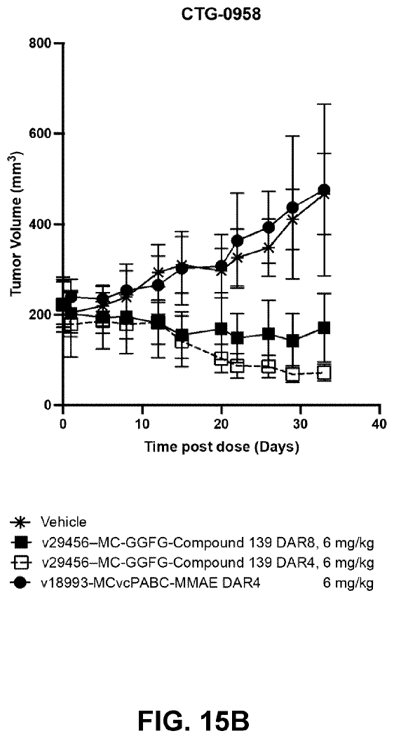

The results for the CTG-2025 study are shown in FIG. 15A. At 6 mg/kg, v29456-MC-GGFG-AM-Compound 139 resulted in a strong inhibition of tumor growth at DAR8. In contrast, at 6 mg/kg, v29456-MC-GGFG-AM-Compound 139 was inactive at DAR4, as was v18993 (lifastuzumab)-MCvcPABC-MMAE.

The results for the CTG-0958 study are shown in FIG. 15B. At 6 mg/kg, v29456-MC-GGFG-AM-Compound 139 resulted in a strong inhibition of tumour growth at a DAR of 4 or 8. For comparison, v18993 (lifastuzumab)-MCvcPABC-MMAE was inactive at 6 mg/kg.

### Example 26: Pharmcokinetic Assessment of ADCs in Tg32 Mice

The pharmacokinetics (PK) of v29456 and ADCs comprising v29456 conjugated to Compound 139 or Compound 141 at a DAR4 and DAR8 was assessed in humanized FcRn Tg32 mice. This mouse model can be predictive of the pharmacokinetics of a drug in humans (see Avery et al. (2016) Utility of a human FcRn transgenic mouse model in drug discovery for early assessment and prediction of human pharmacokinetics of monoclonal antibodies, mAbs, 8:6, 1064-1078). PK of the MX35 antibody (v18992) and of v18993 (lifastuzumab)-MCvcPABC-MMAE DAR4 were assessed for comparison.

All test articles were administered at 5 mg/kg to hFcRn Tg32 mice (The Jackson Laboratory, Sacramento, CA; Stock #014565) by intravenous injection as shown in Table 26.1. For each test article, blood was collected from n=4 animals by retro-orbital bleed at 1, 3, and 6 hours and 1, 3, 7, 10, 14, 21 days post-dose. Blood was processed to serum and stored frozen at -80° C. in 96-well storage plates prior to pharmacokinetic analysis.

TABLE 26.1
Test articles and doses for PK assessment

| Group | Test Article | DAR | Dose (mg/kg) |
|---|---|---|---|
| 1 | v29456 | / | 5 |
| 2 | v29456-MC-GGFG-AM-Compound 139 | 8 | 5 |
| 3 | v29456-MC-GGFG-AM-Compound 139 | 4 | 5 |
| 4 | v29456-MC-GGFG-AM-Compound 141 | 8 | 5 |
| 5 | v29456-MC-GGFG-AM-Compound 141 | 4 | 5 |
| 6 | v18992 (MX35) | 1 | 5 |
| 7 | v18993 (lifastuzumab)-MCvcPABC-MMAE | 14 | 5 |

Test article in mouse serum were captured onto a 384 well plate coated with Goat anti-Human IgG Fc antibody (Jackson 109-005-098). The total antibody was detected with a goat anti-Human IgG Fab Biotin antibody (Jackson 109-065-097), followed by streptavidin SULFO-TAG (Mesoscale). After addition of MSD GOLD Read Buffer A, electrochemiluminescence (ECL) signal by the SULFO-TAG label was measured using the plate reader (Mesoscale).

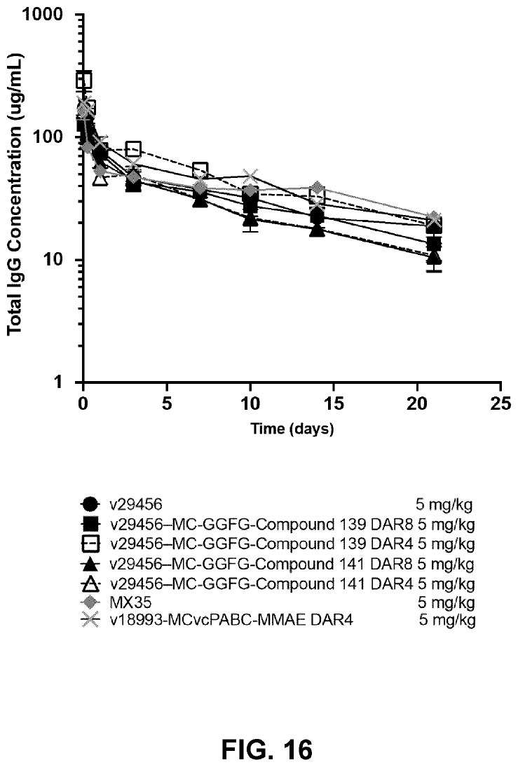

The PK profiles obtained are shown in FIG. 16. All v29456 ADCs and mAbs assessed demonstrated a typical antibody PK profile that was largely comparable between test articles and to the MX35 and lifastuzumab-MMAE comparators. The PK profiles of v29456 humanized antibody variant and its ADCs were comparable.

### Example 27: Functional Characterization of Anti-NaPi2B ADCS—Internalization

Internalization of humanized anti-NaPi2b antibody v29456 (H1L2) conjugated to drug linker MT-GGFG-AM-Compound 139 at DAR 8, in NaPi2b-expressing cell lines (IGROV-1 and OVCAR-3) was determined by flow cytometry as described in Example 12. The NaPi2b-targeting antibodies lifastuzumab (v18993) and MX35 (v18992) were used as positive controls, and palivizumab (anti-RSV) (v22277) was used as a negative control.

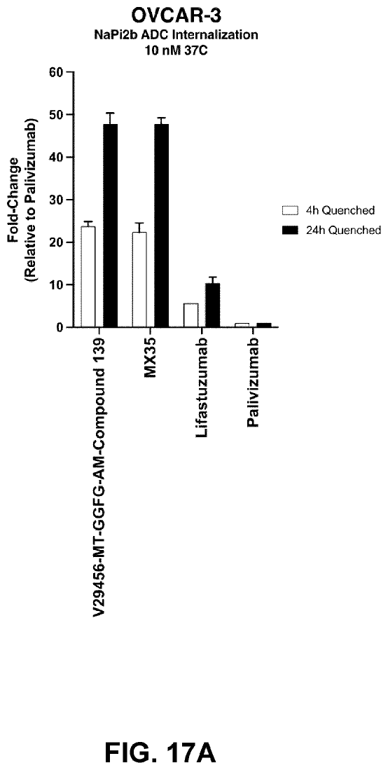

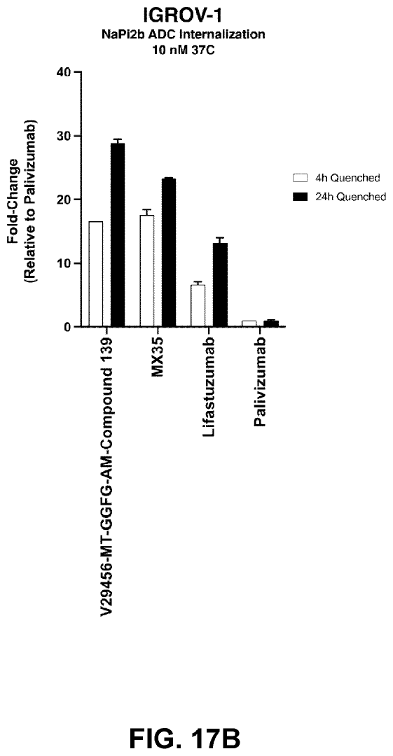

Results are shown in FIG. 17A (OVCAR-3 cells) and FIG. 17B (IGROV-1 cells), and in Table 27.1 below.

TABLE 27.1
Internalization of ADC and Antibody
Constructs in Ovarian Cancer Cell Lines

| | Internalized Fluorescence Fold-Over Palivizumab | | | | | | | |
|---|---|---|---|---|---|---|---|---|
| | IGROV-1 | | | | OVCAR-3 | | | |
| Sample (10 nM) | 4 hrs | | 24 hrs | | 4 hrs | | 24 hrs | |
| v29456-MT- | 16.7 | 16.6 | 28.4 | 29.3 | 23.0 | 24.6 | 49.6 | 45.9 |
| GGFG-AM- | | | | | | | | |
| Compound | | | | | | | | |
| 139 DAR 8 | | | | | | | | |
| v18992 (MX35) | 17.0 | 18.2 | 23.4 | 23.3 | 23.9 | 20.9 | 48.8 | 46.7 |
| v18993 | 7.0 | 6.4 | 13.8 | 12.7 | 5.7 | 5.7 | 9.5 | 11.4 |
| (lifastuzumab) | | | | | | | | |
| v22277 (neg | 1.0 | 1.0 | 1.1 | 0.9 | 1.0 | 1.0 | 1.0 | 1.0 |
| control) | | | | | | | | |

Humanized antibody variant v29456 covalently conjugated to drug linker MT-GGFG-AM-Compound 139 showed comparable levels of internalization to the humanized antibody MX35 (v18992) and much greater levels of internalization compared to humanized antibody lifastuzumab (v18993) across all time points (4 hours and 24 hours) at 10 nM antibody treatment on both IGROV-1 and OVCAR-3. Following a 4-hour incubation in IGROV-1, v29456-MT-GGFG-AM-Compound 139 showed 16.6 and 16.7-fold increase in internalized fluorescence compared to negative control palivizumab, respectively. Similarly, following a 4-hour incubation in OVCAR-3 cells, v29456-MT-GGFG-AM-Compound 139 showed 23.0- and 24.6-fold increase in internalized fluorescence compared to negative control palivizumab, respectively.

Internalization and co-localization of parent antibody v29456 with lysosomes was also assessed in IGROV-1 cells by high content imaging. The data obtained showed that anti-NaPi2b antibody v29456 co-localized with lysosomes after a 24 hour incubation period (data not shown).

### Example 28: In Vitro Cytotoxicity of Camptothecin Analogue Antibody-Drug Conjugates—3D Spheroids

The cell growth inhibition (cytotoxicity) capabilities of the humanized variant v29456 conjugated to MT-GGFG-AM-Compound 139, at DAR8, was assessed against NaPi2b-expressing cell line spheroids according to the methods described in Example 22. Cell lines used were IGROV-1 (ovarian adenocarcinoma), NCI-H441 (lung carcinoma), and TOV-21G (ovarian adenocarcinoma) in monoculture (3,000 cells per well). Reference antibody lifastuzumab conjugated to MMAE was tested as a comparator. ADCs with anti-RSV antibody palivizumab (v21995) were used as non-targeted controls.

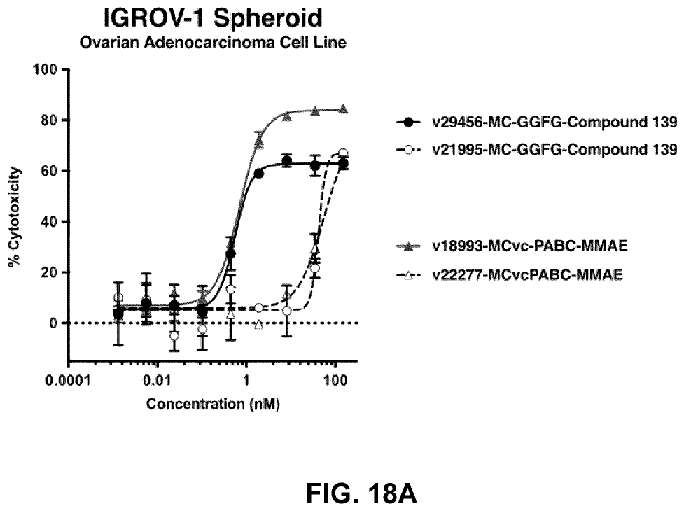

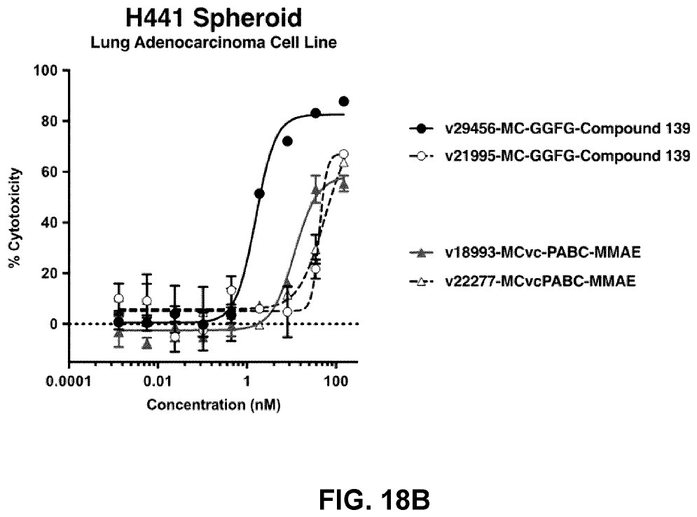

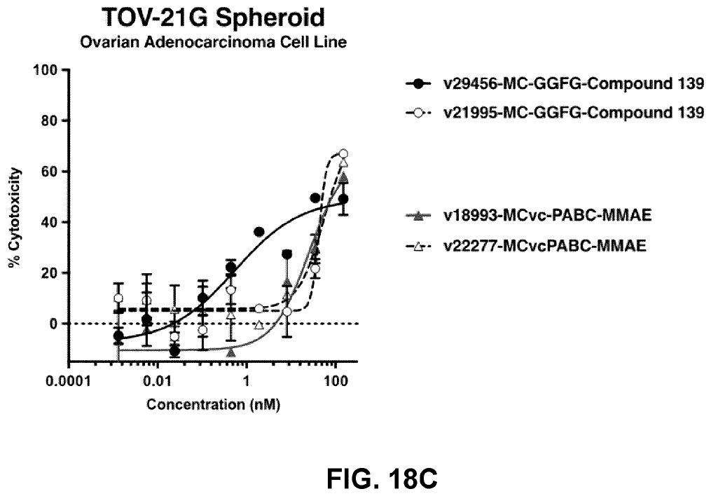

The results are shown in Table 28.1 and representative curves are plotted in FIG. 18A (IGROV-1), FIG. 18B (H441) and FIG. 18C (TOV-21G).

TABLE 28.1
In vitro Cytotoxicity-3D Spheroids-Comparison to lifastuzumab ADC

| | | EC50 (nM) | | |
|---|---|---|---|---|
| | | IGROV-1 | TOV-21G | H441 |
| Sample | DAR | Spheroids | Spheroids | Spheroids |
| v29456-MC-GGFG- | 8.0 | 0.55 | 0.54 | 1.57 |
| AM-Compound 139 | | | | |
| v21995-MC-GGFG- | 8.0 | 42.27 | >150 | 24.52 |
| AM-Compound 139 | | | | |
| v18993 (lifastuzumab)-MCvc- | 4.0 | 0.69 | 25.53 | 11.66 |
| PABC-MMAE | | | | |
| v22277-MCvcPABC-MMAE | 4.0 | 54.61 | >150 | >150 |

v29456-MC-GGFG-AM-Compound 139 showed targeted killing against NaPi2b-expressing spheroids, with sub-nanomolar to single-digit nanomolar EC50, while negative control palivizumab ADC showed double-digit nanomolar or lower potency. v29456-MC-GGFG-AM-Compound 139 showed greater potency than the lifastuzumab-MCvcPABC-MMAE comparator in TOV21-G and NCI-H441 tumor spheroids.

### Example 29: Assessment of Specificity of Anti-NaPi2B Antibody

A Membrane Proteome Array™ (Integral Molecular, Philadelphia, PA, USA) was used to screen for specific off-target binding interactions for antibody, humanized v38591 anti-NaPi2b (SLC34A2) variant. This anti-NaPi2b humanized antibody variant has amino acid sequences that are identical to v29456, except that the heavy chains of v38591 include a C-terminal lysine.

Briefly, the study consisted of three phases: phase (1) determination of assay screening conditions, phase (2) membrane proteome array (library) screen and phase (3) protein target validation. In phase (1), conditions appropriate for detecting v38591 binding by high-throughput flow cytometry were determined, including the optimal antibody concentration and cell type for screening (two cell types were tested, HEK293T and avian QT6). In phase (2), using optimal conditions determined in phase 1, v38591 was screened against the library of over 6000 human membrane proteins (individually expressed in unfixed HEK293T cells), including 94% of all single-pass, multi-pass, and GPI-anchored proteins, including GPCRs, ion channels and transporters. In phase (3), each protein target hit from the screen stage (potential off-target interactions) was assessed in titration experiment using flow cytometry.

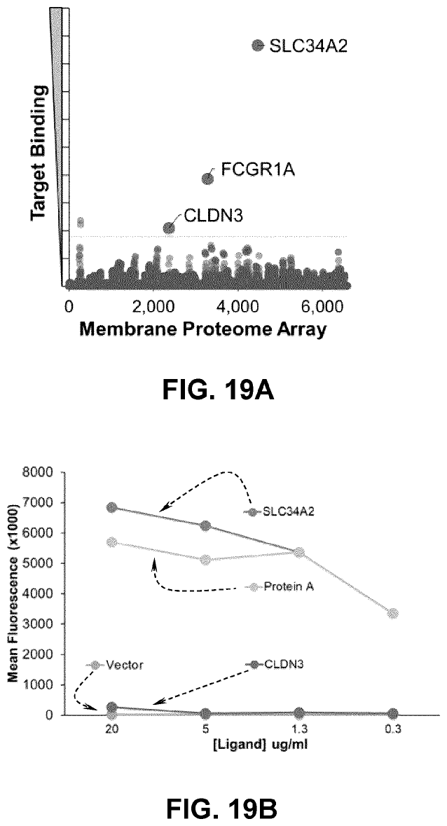

Phase (1) determined that the HEK293T cell type and an antibody concentration of 20 μg/mL were optimal for library screening. As shown in FIG. 19A, library screening resulted in validated protein target hits for the primary target of NaPi2b as well as for FcgR1A which binds the Fc portion of the antibody. Another validated protein target hit was CLDN3. CLDN3 validation data indicated it was a very weak binder to humanized v38591, as shown by a low binding signal to v38591 across a range of concentrations (MFI-60-275), compared to a strong binding signal in the case of NaPi2b (MFI~3500-7000) in the validation assay (FIG. 19B). In general, this data is indicative of high specificity of humanized v38591 for the primary target, NaPi2b.

### Example 30: Functional Characterization of Anti-NaPi2B Antibody-Drug Conjugate—Cellular Binding of Anti-NaPi2B Antibody by Flow Cytometry

The ability of the humanized antibody variant v38591 and the antibody drug conjugates v38591-MC-GGFG-AM-Compound 139 DAR 8 and v38591-MC-GGFG-AM-Compound 139 DAR 4 to bind to NaPi2b expressed on cells was assessed on the endogenous NaPi2b-expressing tumor cell lines IGROV-1 (ovarian adenocarcinoma) and OVCAR-3 (ovarian adenocarcinoma) by flow cytometry. IGROV-1 and OVCAR-3 cells express endogenous NaPi2b at high levels, as described in Example 15. v38591-MC-GGFG-AM-Compound 139 DAR 8 and v38591-MC-GGFG-AM-Compound 139 DAR 4 were prepared similarly to the methods described for v29456-MC-GGFG-AM-Compound 139 DAR 8 and v29456-MC-GGFG-AM-Compound 139 DAR 4 in Example 17.

Cells were maintained under standard culture conditions (37° C./5% CO₂) until assay set-up; IGROV-1 cells were cultured in RPMI 1640, ATCC modification (Thermo Fisher Scientific, Waltham, MA) supplemented with 10% fetal bovine serum (Thermo Fisher Scientific, Waltham, MA); OVCAR-3 cells were cultured in RPMI 1640, ATCC modification (Thermo Fisher Scientific, Waltham, MA) supplemented with 20% fetal bovine serum (Thermo Fisher Scientific, Waltham, MA), and 0.01 μg/mL human insulin (Sigma-Aldrich, Oakville, Canada); Cells were removed from culture vessels using Cell Dissociation Buffer (Invitrogen, Waltham, MA), seeded at 50,000 cells/well in conical-bottom 96-well plates, and treated with test antibody or antibody-drug conjugate for 24 hours at 4° C. to prevent internalization. Reference anti-NaPi2b antibodies MX35 (v18992) and lifastuzumab (v18993) were included as positive controls; palivizumab (anti-RSV antibody, v22277) was included as a negative control. Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal geometric mean, proportional to anti-Human AF647 binding) was calculated for the live cell population using FlowJo™ Version 10.8.1 (BD Biosciences, Franklin Lake, NJ) and plotted for each test antibody or antibody-drug conjugate using GraphPad Prism Version 9 (GraphPad Software, San Diego, CA).

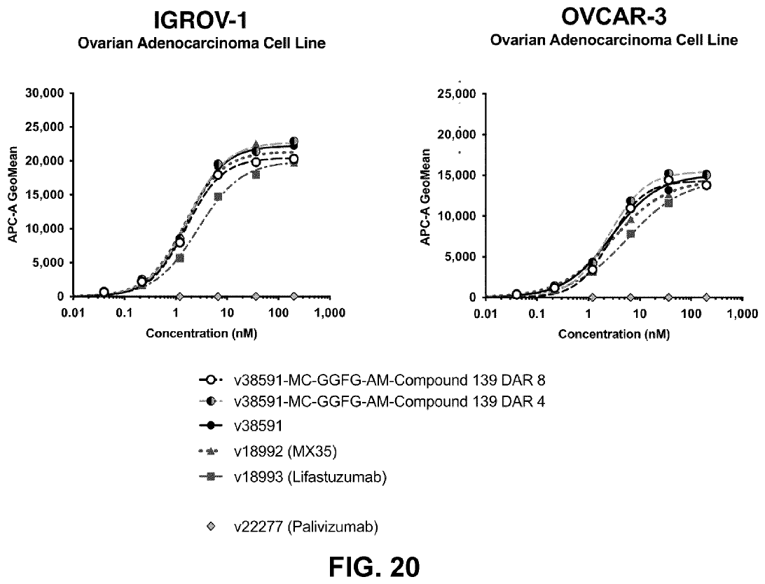

Results are shown in Table 30.1 and plotted in FIG. 20. Both v38591-MC-GGFG-AM-Compound 139 DAR 8 and DAR 4 demonstrated comparable cellular binding to parental antibody v38591 on IGROV-1 and OVCAR-3 cell lines, indicating that conjugation of naked antibody to MC-GGFG-AM-Compound 139 had minimal impact on the cellular binding capability of the antibody. Reference anti-NaPi2b antibody v18992 (MX35) showed similar cellular binding to v38591; reference anti-NaPi2b antibody v18993 (lifastuzumab) showed nearly two-fold lower binding by Kd and decreased Bmax ceiling than v38591, on both IGROV-1 and OVCAR-3. Negative control palivizumab (v22277) showed no cellular binding, as expected.

TABLE 30.1
Cellular Binding of v38591 ADCs, Parental Antibody, and Controls

| | | IGROV-1 | | | OVCAR-3 | | |
|---|---|---|---|---|---|---|---|
| | | | | Curve | | | Curve |
| | | | | Hill | | | Hill |
| | | | Kd | Slope | | Kd | Slope |
| Sample | DAR | Bmax | (nM) | (h) | Bmax | (nM) | (h) |
| v38591-MC-GGFG-AM- | 8 | 20,465 | 1.60 | 1.2 | 14,334 | 2.71 | 1.3 |
| Compound 139 | | | | | | | |
| v38591-MC-GGFG-AM- | 4 | 22,727 | 1.70 | 1.2 | 15,479 | 2.52 | 1.2 |
| Compound 139 | | | | | | | |
| v38591 | — | 22,247 | 1.67 | 1.7 | 15,064 | 2.80 | 1.0 |
| v18992 (MX35) | — | 21,361 | 1.49 | 1.5 | 14,476 | 3.03 | 0.8 |
| v18993 (lifastuzumab) | — | 19,952 | 2.69 | 2.7 | 14,524 | 5.79 | 0.8 |
| v22277 (palivizumab) | — | NB | NB | NB | NB | NB | NB |

* NB = no binding (apparent Kd value greater than the highest antibody testing concentration >200 nM)

### Example 31: Functional Characterization of Anti-NaPi2B Antibody-Drug Conjugate—Cynomolgus and Mouse NaPi2B Binding

The binding cross-reactivity of humanized antibody variant v38591 and the antibody drug conjugates v38591-MC-GGFG-AM-Compound 139 DAR 8 and v38591-MC-GGFG-AM-Compound 139 DAR 4 to cynomolgus monkey and mouse NaPi2b was assessed by flow cytometry using HEK293-6e transfected cells. Reference anti-NaPi2b antibodies MX35 (v18992) and lifastuzumab (v18993) were included as positive controls; palivizumab (anti-RSV antibody, v22277) was included as a negative control.

HEK293-6e cells were maintained under standard culture conditions (37° C./5% CO₂) with shaking at 115 rpm for suspension and cultured in FreeStyle™ 293 Expression Medium (Thermo Fisher Scientific, Waltham, MA) supplemented with 1% fetal bovine serum (Thermo Fisher Scientific, Waltham, MA) and 1× Penicillin-Streptomycin (Thermo Fisher Scientific, Waltham, MA). Cells were transfected with human NaPi2b (pTT5-huNaPi2b) (CL_#13432), cynomolgus (CL_#13433) or mouse (CL #13476) NaPi2b (all from GenScript Biotech, Piscataway, NJ), 1 μg of DNA per 1 million cells, using 293Fectin™ Transfection Reagent (Thermo Fisher Scientific, Waltham, MA) and Opti-MEM™ I Reduced Serum Medium (Thermo Fisher Scientific, Waltham, MA), and incubated for 24 hours under standard culture conditions (37° C./5% CO2/115 rpm). Following transfection, cells were seeded at 50,000 cells/well in conical-bottom 96-well plates and treated with test antibody or antibody-drug conjugate for 24 hours at 4° C. to prevent internalization. Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal geometric mean, proportional to anti-Human AF647 binding) was calculated for the live cell population using FlowJo™ Version 10.8.1 (BD Biosciences, Franklin Lake, NJ) and plotted for each test antibody or antibody-drug conjugate using GraphPad Prism Version 9 (GraphPad Software, San Diego, CA).

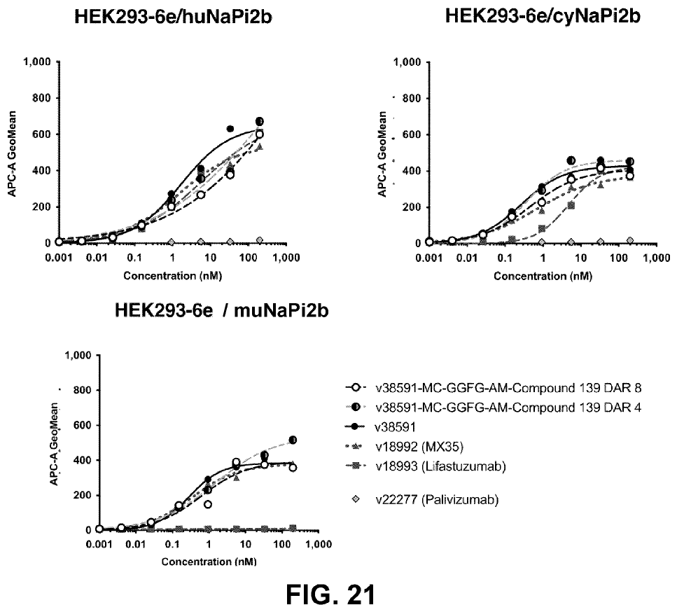

Results are shown in Table 31.1 and plotted in FIG. 21. Antibody-drug conjugates v38591-MC-GGFG-AM-Compound 139 at DAR 8 and at DAR 4, antibody variant v38591 and reference antibody v18992, displayed comparable binding to HEK293-6e cells transfected with human, cynomolgus monkey, and mouse NaPi2b. Lifastuzumab (v18993) displayed poorer binding to cynomolgus NaPi2b-transfected HEK293-6e than all other test antibodies, and minimal binding to mouse NaPi2b-transfected HEK293-6e cells. Negative control palivizumab (v22277) showed no cellular binding, as expected.

TABLE 31.1
Cross-reactivity of v38591 ADCs, parental antibody, and controls, to cynomolgus
monkey and mouse NaPi2b

| | | Bmax | | | Apparent Kd (nM) | | |
|---|---|---|---|---|---|---|---|
| | | Hu | Cy | | Hu | Cy | Ms |
| Sample | DAR | NaPi2b | NaPi2b | Ms NaPi2b | NaPi2b | NaPi2b | NaPi2b |
| v38591-MC-GGFG- | 8 | 1,530 | 408 | 396 | IC | 0.47 | 0.64 |
| AM-Compound 139 | | | | | | | |
| v38591-MC-GGFG- | 4 | 1,256 | 459 | 538 | IC | 0.36 | 1.49 |
| AM-Compound 139 | | | | | | | |
| v38591 | — | 651 | 429 | 385 | 1.68 | 0.27 | 0.29 |
| v18992 (MX35) | — | 548 | 378 | 379 | 1.51 | 0.67 | 0.35 |
| v18993 (lifastuzumab) | — | 678 | 424 | NB | 5.76 | 4.71 | NB |
| v22277 (palivizumab) | — | NB | NB | NB | NB | NB | NB |

NB = no binding (apparent Kd value greater than the highest antibody testing concentration >200 nM)
IC = incomplete curve

### Example 32: Functional Characterization of Anti-NaPi2B Antibody-Drug Conjugate—NaPi2B Specificity

The binding cross-reactivity of humanized antibody variant v38591 and the antibody drug conjugates v38591-MC-GGFG-AM-Compound 139 DAR 8 and v38591-MC-GGFG-AM-Compound 139 DAR 4 to human NaPi2b, NaPi2a, and NaPi2c was assessed by flow cytometry using HEK293-6e transfected cells. Reference anti-NaPi2b antibodies MX35 (v18992) and lifastuzumab (v18993) were included as positive controls; reference anti-NaPi2a (polyclonal rabbit anti-human SLC34A1; Atlas Biotechnologies Inc, Edmonton, AB; Cat. No. HPA051255) and anti-NaPi2c (polyclonal rabbit anti-human SLC34A3; Thermo Fisher Scientific, Waltham, MA; Cat. No. PA5-50762) antibodies were included; palivizumab (anti-RSV antibody, v22277) was included as a negative control.

HEK293-6e cells were maintained under standard culture conditions (37° C./5% CO₂) with shaking at 110 rpm for suspension and cultured in FreeStyle™ 293 Expression Medium (Thermo Fisher Scientific, Waltham, MA) supplemented with 1% fetal bovine serum (Thermo Fisher Scientific, Waltham, MA) and 1× Penicillin-Streptomycin (Thermo Fisher Scientific, Waltham, MA). Cells were transfected with human NaPi2b (pTT5-huNaPi2b) (CL_#13432), human NaPi2a (CL #13435), or human NaPi2c (CL_#13436) (all from GenScript Biotech, Piscataway, NJ), 1 μg of DNA per 1 million cells, using 293Fectin™ Transfection Reagent (Thermo Fisher Scientific, Waltham, MA) and Opti-MEM™ I Reduced Serum Medium (Thermo Fisher Scientific, Waltham, MA), and incubated for 24 hours under standard culture conditions (37° C./5% CO2/110 rpm). Following transfection, cells were seeded at 50,000 cells/well in conical-bottom 96-well plates and treated with test antibody or antibody-drug conjugate for 24 hours at 4° C. to prevent internalization. Following incubation, cells were washed and stained with anti-Human IgG Fc AF647 conjugate (Jackson Immuno Research Labs, West Grove, PA; Cat. No. 109-605-098) or anti-Rabbit IgG F(ab″)₂ AF647 conjugate (Jackson Immuno Research Labs, West Grove, PA; Cat. No. 111-605-047). Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal geometric mean, proportional to anti-Human AF647 binding) was calculated for the live cell population using FlowJo™ Version 10.8.1 (BD Biosciences, Franklin Lake, NJ) and plotted for each test antibody or antibody-drug conjugate using GraphPad Prism Version 9 (GraphPad Software, San Diego, CA).

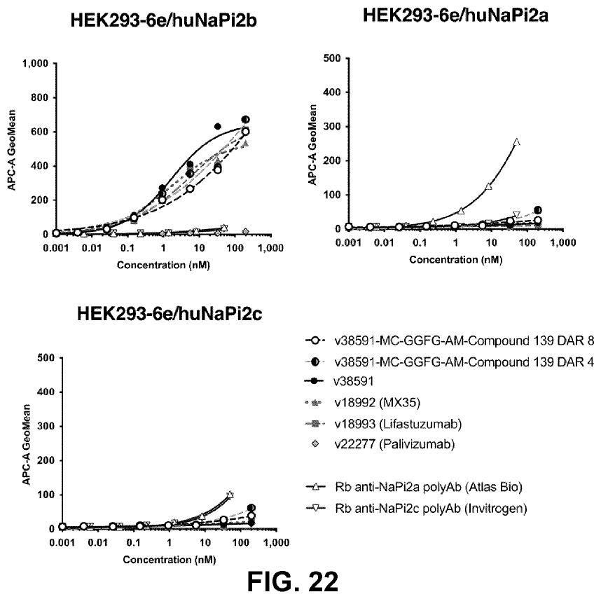

Results are shown in Table 32.1 and plotted in FIG. 22. Antibody-drug conjugate v38591-MC-GGFG-AM-Compound 139 at DAR 8 and at DAR 4, antibody variant v38591, and reference antibodies v18993 and v18992, displayed comparable binding to each other on HEK293-6e cells transfected with NaPi2b, and minimal binding to NaPi2a-transfected and NaPi2c-transfected HEK293-6e cells. Positive NaPi2a-binding control antibody from Atlas Bio demonstrated binding on NaPi2a-transfected cells, some binding to NaPi2c-transfected cells, and no binding to NaPi2b-transfected cells. Positive NaPi2c-binding control antibody from Thermo Fisher Scientific demonstrated some binding to NaPi2c-transfected cells, but not NaPi2a- or NaPi2b-transfected cells, as expected. Negative control Palivizumab (v22277) showed no cellular binding, as expected.

TABLE 32.1
Cross-reactivity of v38591 ADCs, parental antibody, and controls to cynomolgus
monkey and mouse NaPi2b

| | | Bmax | | | Apparent Kd (nM) | | |
|---|---|---|---|---|---|---|---|
| | | Hu | Hu | Hu | Hu | Hu | Hu |
| Sample | DAR | NaPi2b | NaPi2a | NaPi2c | NaPi2b | NaPi2a | NaPi2c |
| v38591-MC-GGFG-AM- | 8 | 1,530 | NB | NB | IC | NE | NB |
| Compound 139 | | | | | | | |
| v38591-MC-GGFG-AM- | 4 | 1,256 | NB | NB | IC | NE | NB |
| Compound 139 | | | | | | | |
| v38591 | — | 651 | NB | NB | 1.68 | NB | NB |
| v18992 (MX35) | — | 548 | NB | NB | 1.51 | NB | NB |
| v18993 (Lifastuzumab) | — | 678 | NB | NB | 5.76 | NB | NB |
| v22277 (Palivizumab) | — | NB | NB | NB | NB | NB | NB |
| anti-SLC34A1 (Atlas | — | NB | IC | IC | NB | IC | IC |
| Biotechnologies Inc.) | | | | | | | |
| anti-SLC34A3 (Thermo | — | NB | NB | IC | NB | NB | IC |
| Fisher Scientific) | | | | | | | |

NB = no binding (apparent Kd value greater than the highest antibody testing concentration >200 nM for human antibodies, >50 nM for rabbit antibodies)
IC = incomplete curve

### Example 33: Functional Characterization of Anti-NaPi2B ADCs -Internalization

Internalization of the anti-NaPi2b ADC v38591-MC-GGFG-AM-Compound 139 DAR 8 and the unconjugated humanized antibody v38591 was determined by flow cytometry in a high NaPi2b-expressing ovarian adenocarcinoma cell line, OVCAR-3, as described below. The NaPi2b-targeting antibodies lifastuzumab (v18993), MX35 (v18992), and palivizumab (anti-RSV) (v22277) were included as controls.

Briefly, cells were seeded at 50,000 cells/well in RPMI 1640, ATCC modification (Thermo Fisher Scientific, Waltham, MA) supplemented with 10% fetal bovine serum (Thermo Fisher Scientific, Waltham, MA) in 48-well plates and incubated overnight under standard culturing conditions (37° C./5% CO₂) to allow attachment. Antibodies were fluorescently labeled by coupling to anti-Human IgG Fc Fab fragment AF488 conjugate (Jackson Immuno Research Labs, West Grove, PA; Cat. No. 109-547-008) at a 1:1 molar ratio in PBS pH 7.4 (Thermo Fisher Scientific, Waltham, MA; Cat. No. 10010-023), for 24 hours at 4° C. Coupled antibodies were added to cells the following day and incubated under standard culturing conditions for 15 minutes, and 4, 16, and 24 hours to allow for internalization. Following incubation, cells were dissociated, washed, and surface AF488 fluorescence was quenched using an anti-AF488 antibody (Life Technologies, Carlsbad, CA; Cat. No. A-11094) at 200 nM for at least 45 minutes at 4° C. Quenched AF488 fluorescence (internalized fluorescence) was detected by flow cytometry on a BD LSRFortessa™ Cell Analyzer (BD Biosciences, Franklin Lake, NJ) with 1,000 minimum events collected per well. The AF488/FITC-A GeoMean (fluorescence signal geometric mean, proportional to anti-Human Fab AF488 labelling) was calculated for the live single cell population using FlowJo™ Version 10.8.1 (BD Biosciences, Franklin Lake, NJ) and plotted using GraphPad Prism Version 9 (GraphPad Software, San Diego, CA).

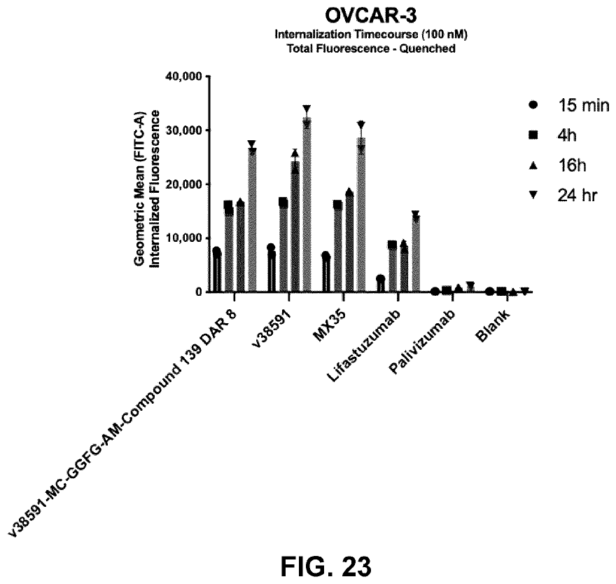

Results are shown in Table 33.1 and FIG. 23. The antibody-drug conjugate, v38591-MC-GGFG-AM-Compound 139 DAR 8, showed comparable levels of internalization to its humanized naked antibody v38591, and v18992 (MX35) control. v38591-MC-GGFG-AM-Compound 139 DAR 8 showed greater levels of internalization compared to v18993 (lifastuzumab) across all time points (15 min, 4 hr, 16 hr and 24 hr) at 100 nM antibody or ADC treatment in OVCAR-3.

TABLE 33.1
Internalized Fluorescence

| | Internalized Fluorescence in OVCAR-3 | | | |
|---|---|---|---|---|
| Sample (100 nM) | 15 min | 4 hr | 16 hr | 24 hr |
| v38593 (v38591-MC-GGFG- | 7377 | 15592 | 16770 | 26670 |
| AM-Compound 139 DAR 8) | | | | |
| v38591 (H1L2) | 7624 | 16587 | 24280 | 32444 |
| v18992 (MX35) | 6652 | 16138 | 18632 | 28643 |
| v18993 (lifastuzumab) | 2483 | 8746 | 8596 | 13878 |
| v22277 (negative control) | 151 | 349 | 854 | 1085 |

### Example 34: Bystander Activity of Anti-NaPi2B ADCs

The ability of the antibody drug conjugates v38591-MC-GGFG-AM-Compound 139 DAR 8 and v38591-MC-GGFG-AM-Compound 139 DAR 4 to exert a bystander killing effect on cancer cells was assessed according to the method described below. Bystander killing can occur after target-specific uptake of an ADC into an antigen-positive cell, as described in Example 19. Cell lines used were HCC-78 (high NaPi2b expression) and EBC-1 (negative NaPi2b expression). Surface NaPi2b expression for these cell lines were measured as described in Example 15.

All test articles were assessed at a concentration of 5 nM. The unconjugated human antibody variant v38591, negative controls palivizumab (v21995) MC-GGFG-AM-Compound 139 DAR 8 and DAR 4, palivizumab (v22277) MC-GGFG-AM-DXd1, and palivizumab (v22277) MCvcPABC-MMAE, were included as controls. The human antibody variant v29456 conjugated to MC-GGFG-AM-DXd1 was included as a positive control. Reference antibody drug conjugate v18993 (lifastuzumab)-MCvcPABC-MMAE was included as a positive control at both 5 nM and 20 nM concentrations (EC₉₉ on HCC-78 cells=20 nM).

Briefly, NaPi2b-positive HCC-78 and NaPi2b-negative EBC-1 cells were seeded either as mono-cultures or co-cultures in a 48-well plate at 15,000 cells and 5,000 cells, respectively, in 100 μL assay media (RPMI1640+10% FBS). ADCs were diluted to 10 nM or 40 nM in assay media and 100 μL was added to the cell-containing plates (5 nM or 20 nM final ADC concentration). Cells were incubated with ADCs for 4 days under standard culturing conditions (37° C./5% CO₂). Following incubation, cells were dissociated, washed, and stained using a viability dye, YO-PRO®-1 (ThermoFisher Scientific, Waltham, MA), and an anti-NaPi2b antibody, MX35, conjugated to Alexa Fluor® 647, for 20 minutes at 4° C. After incubation, cells were washed in FACS buffer, resuspended in 60 μL FACS buffer per well, and 35 μL per well were analyzed on a BD LSRFortessa™ Cell Analyzer (BD Biosciences, Franklin Lake, NJ). Dead cells were excluded by gating on YO-PRO®-1 staining. The number of HCC-78 and EBC-1 cells were determined by the number of events in the Alexa Fluor® 647 positive (NaPi2b-positive) and Alexa Fluor® 647 negative (NaPi2b-negative) gates, respectively. Percent viability was calculated as the number of cells in each treatment condition divided by the number of cells in the no-treatment condition.

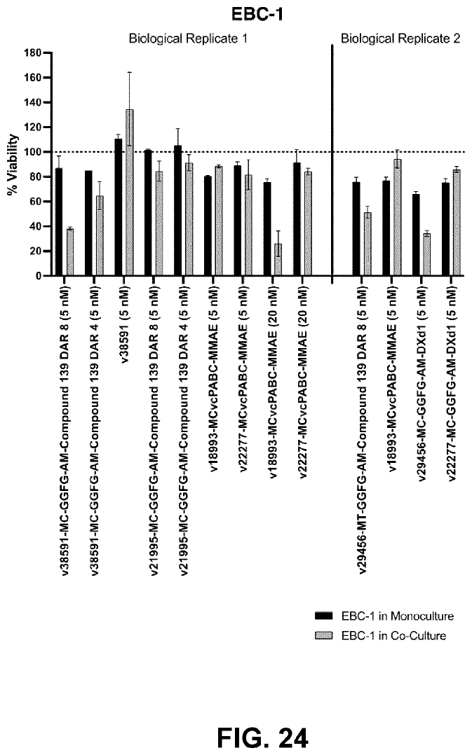

The results are tabulated in Table 34.1 and shown in FIG. 24. Bystander effect was calculated by comparing the viability of NaPi2b-negative EBC-1 cells treated as mono-culture (black bars) with that of the cells treated as a co-culture with NaPi2b-positive HCC-78 cells (grey bars). A greater decrease in viability in co-culture compared with mono-culture indicates a greater bystander effect. Anti-NaPi2b-targeted camptothecin analogue ADCs v38591-MC-GGFG-AM-Compound 139 DAR 8 and v38591-MC-GGFG-AM-Compound 139 DAR 4 demonstrated positive bystander activity, with the DAR 8 showing greater bystander activity than DAR 4. Positive control v29456-MC-GGFG-AM-DXd1 showed bystander activity. Positive control v18993-MCvcPABC-MMAE showed bystander activity when treated at 20 nM, equal to its EC₉₉ against HCC-78. Negative controls v38591, palivizumab MC-GGFG-AM-Compound 139 DAR 8 and DAR 4, palivizumab MC-GGFG-AM-DXd1 and palivizumab MCvcPABC-MMAE displayed minimal killing in EBC-1 in mono-culture or in co-culture, indicating negligible bystander activity, as expected.

TABLE 34.1
Bystander Activity of anti-NaPi2b ADCs against NaPi2b-negative EBC-1 cell line

| | | | Average | Average | |
|---|---|---|---|---|---|
| Biological | | | EBC-1 | EBC-1 % | % |
| Replicate | Sample (treatment | | % viability in | viability in | Bystander |
| Number | concentration) | DAR | mono-culture* | co-culture* | activity |
| Biological | v38591-MC-GGFG-AM- | 8 | 87.1 | 38.1 | 49.0 |
| Replicate 1 | Compound 139 (5 nM) | | | | |
| | v38591-MC-GGFG-AM- | 4 | 85.0 | 64.7 | 20.3 |
| | Compound 139 (5 nM) | | | | |
| | v38591 (5 nM) | — | 110.7 | 134.5 | −23.8 |
| | v21995 (palivizumab)-MC- | 8 | 101.4 | 84.4 | 17.1 |
| | GGFG-AM-Compound 139 | | | | |
| | (5 nM) | | | | |
| | v21995 (palivizumab)-MC- | 4 | 105.4 | 91.3 | 14.1 |
| | GGFG-AM-Compound 139 | | | | |
| | (5 nM) | | | | |
| | v18993 (lifastuzumab)- | 4 | 80.5 | 88.5 | −7.9 |
| | MCvcPABC-MMAE | | | | |
| | (5 nM) | | | | |
| | v22277 (palivizumab)- | 4 | 89.2 | 81.7 | 7.5 |
| | MCvcPABC-MMAE | | | | |
| | (5 nM) | | | | |
| | v18993 (lifastuzumab)- | 4 | 75.6 | 26.0 | 49.6 |
| | MCvcPABC-MMAE | | | | |
| | (20 nM) | | | | |
| | v22277 (palivizumab)- | 4 | 91.5 | 84.1 | 7.4 |
| | MCvcPABC-MMAE | | | | |
| | (20 nM) | | | | |
| Biological | v29456-MC-GGFG-AM- | 8 | 75.8 | 51.4 | 24.4 |
| Replicate 2 | Compound 139 (5 nM) | | | | |
| | v18993 (lifastuzumab)- | 4 | 77.0 | 94.3 | −17.3 |
| | MCvcPABC-MMAE | | | | |
| | (5 nM) | | | | |
| | v29456-MC-GGFG-AM- | 8 | 66.1 | 34.3 | 31.8 |
| | DXd1 (5 nM) | | | | |
| | v22277 (palivizumab)-MC- | 8 | 75.2 | 85.9 | −10.6 |
| | GGFG-AM-DXd1 (5 nM) | | | | |

*Relative to untreated blank

### Example 35: In Vivo Efficacy of Anti-NaPi2B ADCs in Patient-Derived (PDX) Xenografts Models

In vivo anti-tumor activities of humanized variant v29456 ADCs were assessed in several patient-derived (PDX) xenograft models. For all ovarian cancer patient-derived xenograft (PDX) models, research level immunohistochemistry (IHC) assessment on same study tissue was performed by staining with a commercial anti-NaPi2b antibody (Clone D6W2G, Rabbit mAb #42299, Cell Signaling Technology). NaPi2b H-scores were determined by a pathologist for all PDX models, as summarized in Table 35.2. H-scores were calculated on a scale from 0-300 using the formula: H-score=(0×P₀)+(1×P₁)+(2×P₂)+(3×P₃), where P₀, P1, P₂, and P₃ represent the percentage of cells with absent, weak, moderate or strong staining, respectively.

For all ovarian cancer PDX models, anti-tumor activity was determined by % tumor growth inhibition (% TGI) calculated as [(1−TVₜᵣₑₐₜₘₑₙₜ/TV_{vehicle})×100] at Day 28, or at the closest evaluable time point, as summarized in Table 35.2.

For the ovarian cancer PDX model CTG-0703, tumor fragments were implanted subcutaneously into female athymic Nude-Foxn1nu mice. When mean tumor volume reached ~240 mm³ the animals were assigned to groups (n=3 per group) and treated on study day 1 with a single IV dose of test articles as shown in Table 35.1.

TABLE 35.1
Treatment groups for PDX xenograft models

| Group | Treatment | DAR | Dose (mg/kg) |
|---|---|---|---|
| 1 | Vehicle | | 0 |
| 2 | v29456-MC-GGFG-AM-Compound 139 | 8 | 6 |
| 3 | v29456-MC-GGFG-AM-Compound 139 | 4 | 6 |
| 4 | v18993-MCvcPABC-MMAE | 4 | 6 |

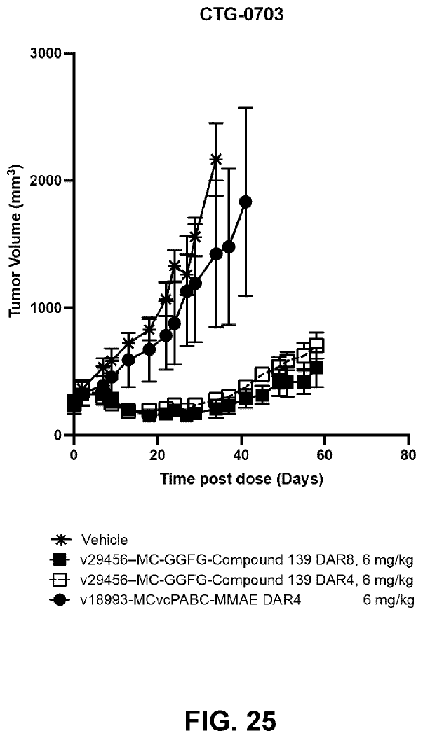

As shown in FIG. 25, at 6 mg/kg, v29456-MC-GGFG-AM-Compound 139 inhibited tumour growth at DAR8 and at DAR4 in the CTG-0703 model. For comparison, v18993-MCvcPABC-MMAE was inactive at 6 mg/kg.

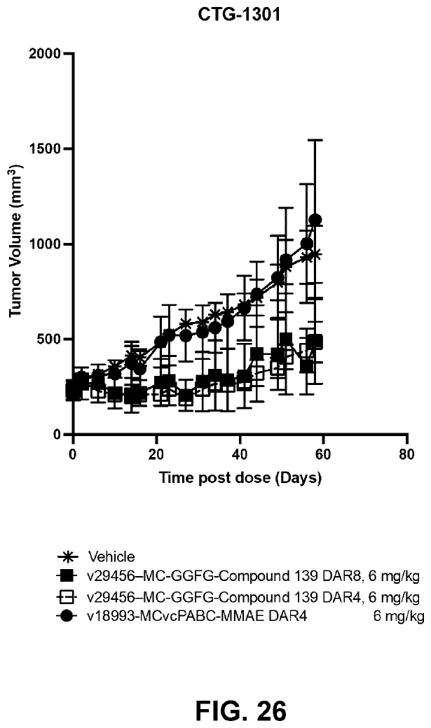

For the ovarian cancer PDX model CTG-1301, tumor fragments were implanted subcutaneously into female athymic Nude-Foxn1nu mice. When mean tumor volume reached ~230 mm³ the animals were assigned to groups (n=3 per group) and treated on study day 1 with a single IV dose of test articles as shown in Table 35.1. As shown in FIG. 26, at 6 mg/kg, v29456-MC-GGFG-AM-Compound 139 inhibited tumour growth at DAR8 and at DAR4. For comparison, v18993-MCvcPABC-MMAE was inactive at 6 mg/kg.

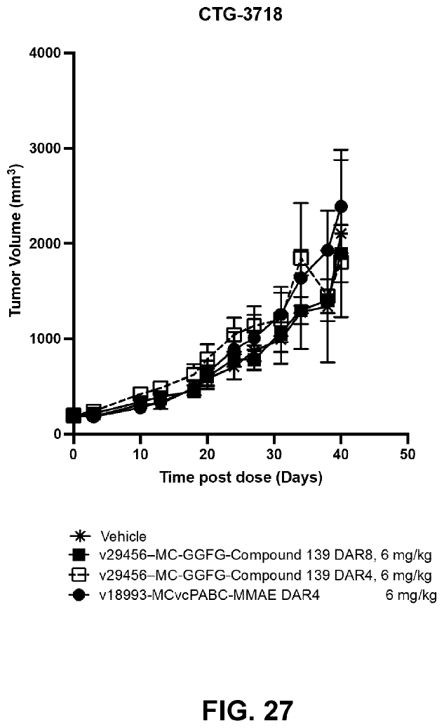

For the ovarian cancer PDX model CTG-3718, tumor fragments were implanted subcutaneously into female athymic Nude-Foxn1nu mice. When mean tumor volume reached ~200 mm³ the animals were assigned to groups (n=3 per group) and treated on study day 1 with a single IV dose of test articles as shown in Table 35.1. As shown in FIG. 27, at 6 mg/kg, v29456-MC-GGFG-AM-Compound 139 was inactive at DAR8 and at DAR4. For comparison, v18993-MCvcPABC-MMAE was also inactive at 6 mg/kg.

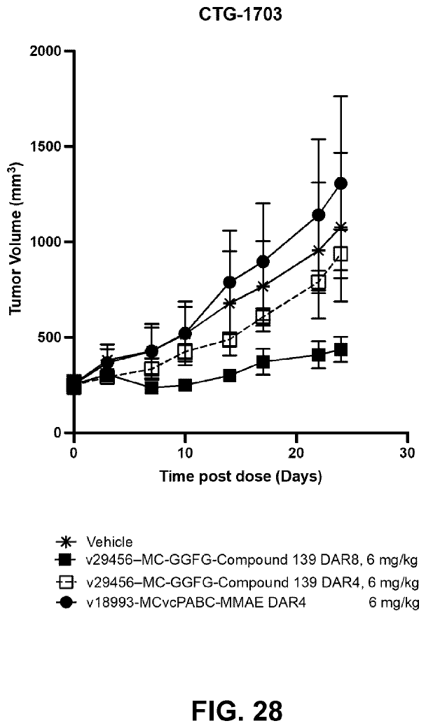

For the ovarian cancer PDX model CTG-1703, tumor fragments were implanted subcutaneously into female athymic Nude-Foxn1nu mice. When mean tumor volume reached ~260 mm³ the animals were assigned to groups (n=3 per group) and treated on study day 1 with a single IV dose of test articles as shown in Table 35.1. As shown in FIG. 28, at 6 mg/kg, v29456-MC-GGFG-AM-Compound 139 was moderately active at DAR8 and inactive at DAR4. For comparison, v18993-MCvcPABC-MMAE was inactive at 6 mg/kg.

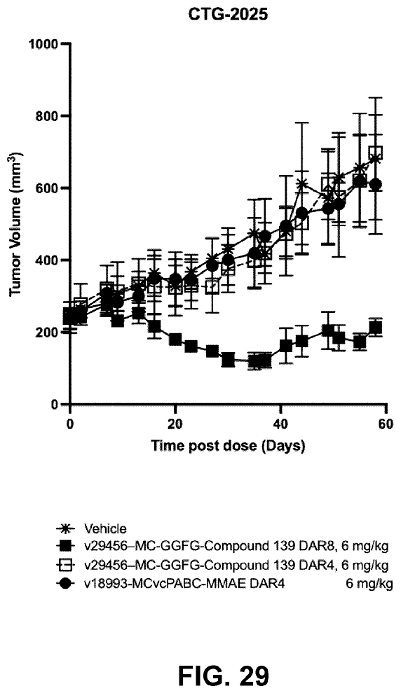

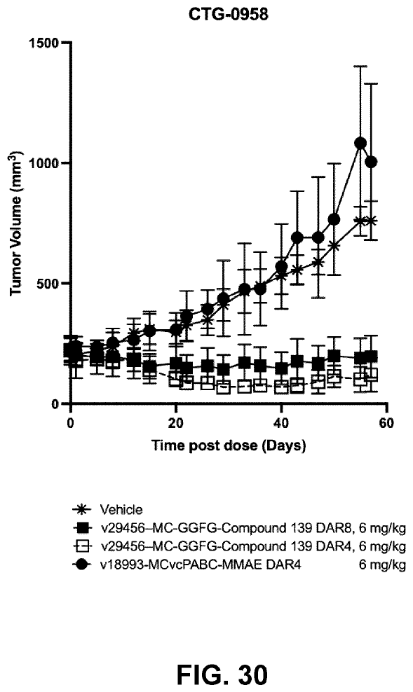

The ovarian cancer PDX models CTG-2025 and CTG-0958 described in Example 25 used the treatment groups shown in Table 35.1. Updated versions of FIG. 15A and FIG. 15B showing treatment out to 60 days are provided in FIG. 29 and FIG. 30, respectively. H-scores were assessed for these models as described above.

As shown in FIG. 29, at 6 mg/kg, v29456-MC-GGFG-AM-Compound 139 inhibited tumour growth at DAR8, and was inactive at DAR4 in the CTG-2025 model. For comparison, v18993-MCvcPABC-MMAE was inactive at 6 mg/kg.

As shown in FIG. 30, at 6 mg/kg, v29456-MC-GGFG-AM-Compound 139 inhibited tumour growth at a DAR of 4 or 8 in the CTG-0958 model. For comparison, v18993-MCvcPABC-MMAE was inactive at 6 mg/kg.

TABLE 35.2
Summary of NaPi2b H-scores and anti-tumor activity in
ovarian cancer PDX models

| | | % Tumor Growth Inhibition (TGI) at Day 28 or | | |
|---|---|---|---|---|
| | | closest evaluablet ime point | | |
| | | v29456-MC- | v29456-MC- | v18993- |
| | | GGFG-AM- | GGFG-AM- | MCvcPABC- |
| | | Compound 139 | Compound 139 | MMAE |
| PDX Model | H-Score | DAR 8 | DAR 4 | DAR 4 |
| CTG-2025 | 150 | 63% | 20% | 5% |
| CTG-0958 | 300 | 65% | 83% | −6% |
| CTG-1703 | 195 | 59% | 13% | −21% |
| CTG-0703 | 115 | 88% | 85% | 10% |
| CTG-1301 | 190 | 65% | 68% | 11% |
| CTG-3718 | 150 | 11% | −30% | −15% |

The data provided in Table 35.2 indicates that v29456-MC-GGFG-AM-Compound 139 has anti-tumor activity in ovarian cancer xenograft models with a range of NaPi2b expression levels.

### Example 36: Cynomolgus Monkey and Rat Toxicokinetic Study

The objective of this study was to determine the maximum tolerated dose of test articles v38591-MC-GGFG-AM-Compound 139 (DAR4), and v38591-MC-GGFG-AM-Compound 139 (DAR8) in male cynomolgus monkeys following three repeat slow intravenous bolus injections. In addition, the toxicokinetic (TK) profiles of these ADCs in cynomolgus monkeys was characterized.

In this study, vehicle and test article ADCs were administered by slow intravenous injection over 3 minutes on Day 1, Day 22, and Day 43 to male cynomolgus monkeys (n=3/group). Study design, dose levels and dose volume details are summarized in the Table 36.1. All the animals were evaluated for moribundity/mortality, clinical signs, body weight, food consumption, clinical pathology (hematology, serum chemistry and coagulation), changes in organ weight, and macroscopic and microscopic changes in organs/tissues. Blood samples were collected for toxicokinetic analysis following the first and second dose. The test article concentrations in all dose formulations were analyzed using UV-Vis assay. Scheduled necropsy was conducted on study Day 50.

Total antibody in cynomolgus monkey serum samples was measured by MSD (Mesoscale Discovery), assay details as described in Example 26. Note for cynomolgus monkey serum samples, Goat anti-human IgG (H+L) Biotin antibody was used for antibody capture in MSD assay.

TABLE 36.1
Study Design

| | | | | Dose | | |
|---|---|---|---|---|---|---|
| | | | Dose level | volume | Concentration | Number of |
| Group | Treatment | DAR | (mg/kg/dose) | (mL/kg) | (mg/mL) | animals |
| 1 | Vehicle | | 0 | 5 | 0 | 3 |
| 2 | v38591-MC-GGFG- | 8 | 15 | 5 | 3 | 3 |
| 3 | AM-Compound 139 | | 30 | 5 | 6 | 3 |
| 4 | | | 45 | 5 | 9 | 3 |
| 5 | v38591-MC-GGFG- | 4 | 30 | 5 | 6 | 3 |
| 6 | AM-Compound 139 | | 60 | 5 | 12 | 3 |
| 7 | | | 90 | 5 | 18 | 3 |

v38591-MC-GGFG-AM-Compound 139, DAR8: All animals survived to their scheduled euthanasia (Study Day 50). No abnormal functional observational battery, or detailed clinical observations were noted. Test article-related but non-adverse cage-side clinical observations included soft and/or loose feces intermittently throughout the study at 45 mg/kg/day.

Preterminal Animals: Mean body weight gain and mean body weights were comparable to controls and no effect on qualitative food consumption was noted. As compared to animal baseline values and/or historical control data, fibrinogen (FIB) levels were transiently increased on Day 8 for animals administered 45 mg/kg/dose; however, values returned to baseline throughout the remainder of the study. Increases in alanine aminotransferase (ALT) were observed from Day 21 for all dose levels.

Terminal Animals: A single animal administered 30 mg/kg/dose was noted with the macroscopic observation of thickened intestinal wall of the duodenum. Dose-responsive decrease in absolute, organ to body weight, and organ to brain weight ratios were noted in the testes and epididymides of animals administered 15, 30, or 45 mg/kg/dose. Microscopic examination results are currently pending.

v38591-MC-GGFG-AM-Compound 139, DAR4: All animals survived to their scheduled euthanasia (Study Day 50). No abnormal functional observational battery or detailed clinical observations were noted. Test article-related but non-adverse cage-side clinical observations included soft and/or loose feces intermittently throughout the study at 90 mg/kg/day.

Preterminal Animals: Mean body weight gain and mean body weights were comparable to controls and no effect on qualitative food consumption was noted. As compared to animal baseline values and/or historical control data, fibrinogen (FIB) and lactate dehydrogenase (LDH) levels were transiently increased on Day 4; however, values returned to baseline throughout the remainder of the study.

Terminal Animals: A single animal administered 90 mg/kg/dose was noted with the macroscopic observation of thickened intestinal walls of the cecum, duodenum, and colon. No test article-related organ weight changes were observed. Microscopic examination results are currently pending.

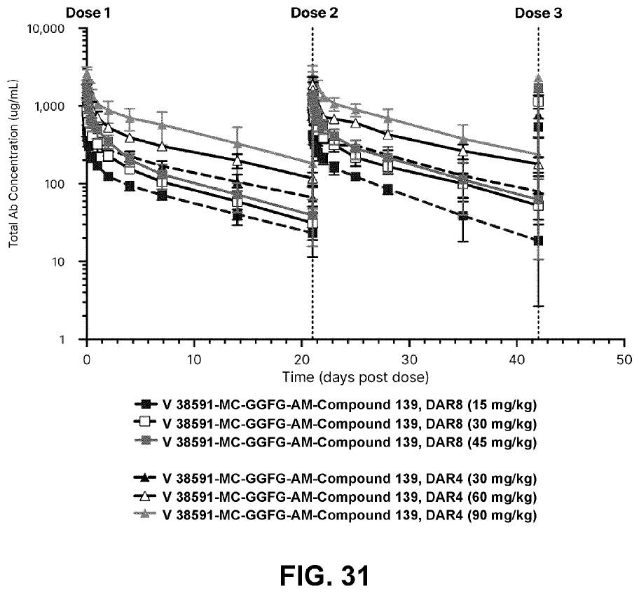

Pharmacokinetic (PK) profile: The PK profiles obtained are shown in FIG. 31. All ADCs assessed demonstrated a typical antibody PK profile. ADC half lives are noted in Table 36.2.

TABLE 36.2
Relevant study parameters from repeat dose toxicology study in cynomolgus
monkeys
NHP 3-Dose Non-GLP Toxicology Study, Q3Wx3

| | Dose | | | | | | | |
|---|---|---|---|---|---|---|---|---|
| | mg/ | | Clinical | Histo- | Clinical | Hema- | | T_{1/2} |
| Test Article | kg | Mortality | Observations | pathology | Chemistry | tology | MTD | (day) |
| v38591-MC- | 15 | None | None | None | None | None | 45 | 8.7 |
| GGFG-AM- | 30 | None | None | None | None | None | mg/kg | |
| Compound | | | | | | | | 7.7 |
| 139, DAR8 | 45 | None | Fecal abnormalities | None | None | None | | |
| | | | (soft/loose) | | | | | 8.0 |
| v38591-MC- | 30 | None | None | None | None | None | 90 | 10.3 |
| GGFG-AM- | 60 | None | None | None | None | None | mg/kg | |
| Compound | | | | | | | | 9.8 |
| 139, DAR4 | | | | | | | | 8.0 |
| | 90 | None | Fecal abnormalities | None | None | None | | |
| | | | (soft/loose/watery) | | | | | |

For the rat study, 4 male and 4 female rats (i.e., a total of 8 animals/cohort) were dosed either 100 mg/kg or 200 mg/kg of v40502 (LALADS)-MC-GGFG-AM-Compound 139 DAR 4 at days 1 and 22, with terminal necropsy at day 28. This study revealed no mortalities or abnormal clinical signs, with a determined half-life in the 200 mg/kg cohort of 11.2 days.

### Example 37: In Vivo Activity of Antibody-Drug Conjugates in Patient-Derived Models of Non-Small Cell Lung Cancer (NSCLC)

In vivo anti-tumor activity of ADCs of humanized variant v38591 (with heavy chains including C-terminal lysine) was assessed in eight patient-derived (PDX) models of NSCLC cancer: LU5213, LU5245, LU6802, LU6904, LU11692, LU11796, LU11870 and LU11876. An ADC of reference antibody v18993 (lifastuzumab) conjugated to MCvcPABC-MMAE at DAR4 was also tested as a comparator in some models. These studies were carried out as described below.

For all NSCLC PDX models, anti-tumor activity was determined by % tumor growth inhibition (% TGI) calculated as [(1−TVₜᵣₑₐₜₘₑₙₜ/TV_{vehicle})×100] at Day 28, or at the closest evaluable time point, as summarized in Table 37.9.

For the NSCLC cancer PDX model LU5213, tumor fragments were implanted subcutaneously into NOD/SCID mice. When mean tumor volume reached ~180 mm³ the animals were assigned to groups (n=3 per group) and treated on study day 0 with a single IV dose of test articles as shown in Table 37.1.

TABLE 37.1
Treatment groups for LU5213 NSCLC model

| Group | Treatment | DAR | Dose (mg/kg) |
|---|---|---|---|
| 1 | Vehicle | — | 0 |
| 2 | v38591-MC-GGFG-AM-Compound 139 | 8 | 6 |
| 3 | v38591-MC-GGFG-AM-Compound 139 | 4 | 6 |

For the NSCLC PDX model LU5245, tumor fragments were implanted subcutaneously into female NOD/SCID mice. When mean tumor volume reached ~160 mm³ the animals were assigned to groups (n=3 per group) and treated on study day 0 with a single IV dose of test articles as shown in Table 37.2.

TABLE 37.2
Treatment groups for LU5245 NSCLC Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v18993 (lifastuzumab)-MCvcPABC-MMAE | 4 | 6 |

For the NSCLC PDX model LU6802, tumor fragments were implanted subcutaneously into female BALB/c nude mice. When mean tumor volume reached ~160 mm³ the animals were assigned to groups (n=3 per group) and treated on study day 0 with a single IV dose of test articles as shown in Table 37.3.

TABLE 37.3
Treatment groups for LU6802 NSCLC Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v38591-MC-GGFG-AM-Compound 139 | 4 | 12 |
| 5 | v18993 (lifastuzumab)-MCvcPABC-MMAE | 4 | 6 |

For the NSCLC PDX model LU6904, tumor fragments were implanted subcutaneously into female BALB/c nude mice. When mean tumor volume reached ~160 mm³ the animals were assigned to groups (n=3 per group) and treated on study day 0 with a single IV dose of test articles as shown in Table 37.4.

TABLE 37.4
Treatment groups for LU6904 NSCLC Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v38591-MC-GGFG-AM-Compound 139 | 4 | 12 |
| 5 | v18993 (lifastuzumab)-MCvcPABC-MMAE | 4 | 6 |

For the NSCLC PDX model LU11692, tumor fragments were implanted subcutaneously into female BALB/c nude mice. When mean tumor volume reached ~175 mm³ the animals were assigned to groups (n=3 per group) and treated on study day 0 with a single IV dose of test articles as shown in Table 37.5.

TABLE 37.5
Treatment groups for LU11692 NSCLC Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v18993 (lifastuzumab)-MCvcPABC-MMAE | 4 | 6 |

For the NSCLC PDX model LU11796, tumor fragments were implanted subcutaneously into female NOD/SCID mice. When mean tumor volume reached ~195 mm³ the animals were assigned to groups (n=3 per group) and treated on study day 0 with a single IV dose of test articles as shown in Table 37.6.

TABLE 37.6
Treatment groups for LU11796 NSCLC Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v18993 (lifastuzumab)-MCvcPABC-MMAE | 4 | 6 |

For the NSCLC PDX model LU11870, tumor fragments were implanted subcutaneously into female NOD/SCID mice. When mean tumor volume reached ~155 mm³ the animals were assigned to groups (n=3 per group) and treated on study day 0 with a single IV dose of test articles as shown in Table 37.7.

TABLE 37.7:
Treatment groups for LU11870 NSCLC Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v18993 (lifastuzumab)-MCvcPABC-MMAE | 4 | 6 |

For the NSCLC PDX model LU11876, tumor fragments were implanted subcutaneously into female NOD/SCID mice. When mean tumor volume reached ~190 mm³ the animals were assigned to groups (n=3 per group) and treated on study day 0 with a single IV dose of test articles as shown in Table 37.8.

TABLE 37.8
Treatment groups for LU11876 NSCLC Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v18993 (lifastuzumab)-MCvcPABC-MMAE | 4 | 6 |

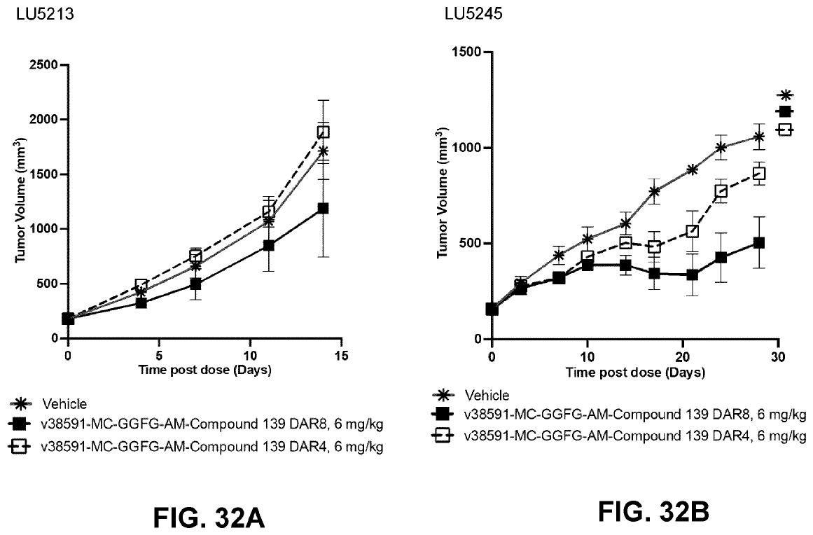

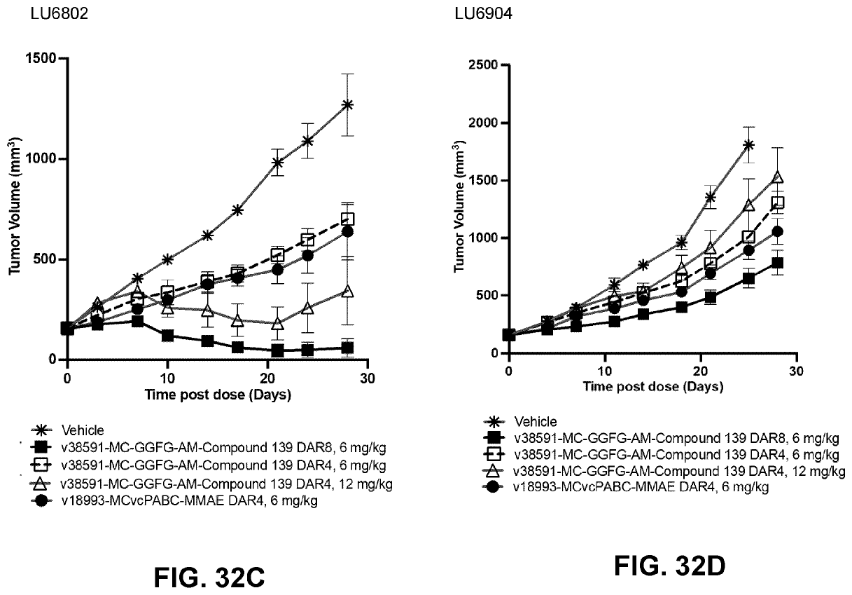

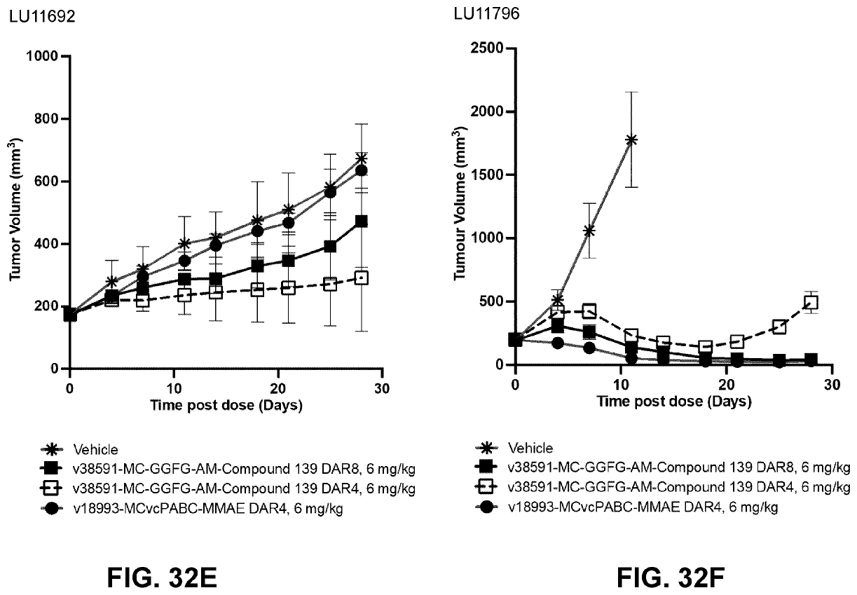

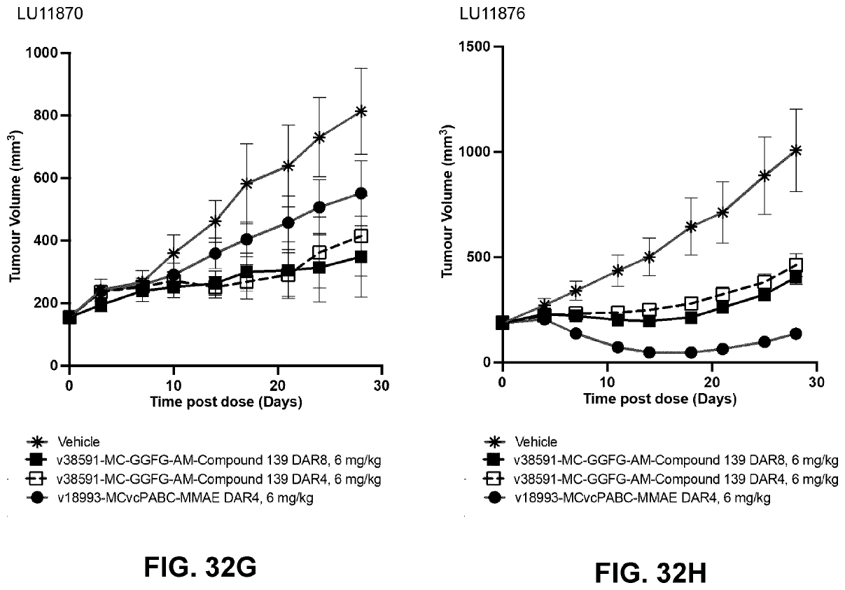

The results for the LU5213 study are shown in FIG. 32A. At 6 mg/kg, neither v38591-MC-GGFG-AM-Compound 139 at DAR8 or DAR4 resulted in substantial inhibition of tumour growth.

The results for the LU5245 study are shown in FIG. 32B. At 6 mg/kg, v38591-MC-GGFG-AM-Compound 139 resulted in a moderate inhibition of tumor growth at a DAR of 4 or 8.

The results for the LU6802 study are shown in FIG. 32C. At a DAR4, v38591-MC-GGFG-AM-Compound 139 resulted in a moderate inhibition of tumor growth at 6 mg/kg and a stronger inhibition of tumor growth at 12 mg/kg. At a DAR8, v38591-MC-GGFG-AM-Compound 139 resulted in a strong inhibition of tumor growth at 6 mg/kg. For comparison, v18993 (lifastuzumab)-MCvcPABC-MMAE resulted in a moderate inhibition of tumor growth at 6 mg/kg.

The results for the LU6904 study are shown in FIG. 32D. At a DAR4, v38591-MC-GGFG-AM-Compound 139 resulted in a moderate inhibition of tumor growth at a 6 mg/kg and 12 mg/kg. At a DAR8, v38591-MC-GGFG-AM-Compound 139 resulted in a moderate inhibition of tumor growth at 6 mg/kg. For comparison, v18993 (lifastuzumab)-MCvcPABC-MMAE resulted in moderate inhibition of tumor growth at 6 mg/kg.

The results for the LU11692 study are shown in FIG. 32E. At 6 mg/kg, v38591-MC-GGFG-AM-Compound 139 resulted in a moderate inhibition of tumor growth at a DAR of 4 or 8. For comparison, v18993 (lifastuzumab)-MCvcPABC-MMAE was inactive at 6 mg/kg.

The results for the LU11796 study are shown in FIG. 32F. At 6 mg/kg, v38591-MC-GGFG-AM-Compound 139 resulted in a strong inhibition of tumor growth at a DAR of 4 or 8. For comparison, v18993 (lifastuzumab)-MCvcPABC-MMAE resulted in a strong inhibition of tumor growth at 6 mg/kg.

The results for the LU11870 study are shown in FIG. 32G. At 6 mg/kg, v38591-MC-GGFG-AM-Compound 139 resulted in a moderate inhibition of tumor growth at a DAR of 4 or 8. For comparison, v18993 (lifastuzumab)-MCvcPABC-MMAE resulted in a moderate inhibition of tumor growth at 6 mg/kg.

The results for the LU11876 study are shown in FIG. 32H. At 6 mg/kg, v38591-MC-GGFG-AM-Compound 139 resulted in a moderate inhibition of tumor growth at a DAR of 4 or 8. For comparison, v18993 (lifastuzumab)-MCvcPABC-MMAE resulted in a strong inhibition of tumor growth at 6 mg/kg.

TABLE 37.9
Summary of anti-tumor activity (% tumor growth inhibition) of
v38591-MC-GGFG-AM-Compound 139 in NSCLC PDX models

| | v38591-MC- | v38591-MC- | v38591-MC- | v18993- |
|---|---|---|---|---|
| | GGFG-AM- | GGFG-AM- | GGFG-AM- | MCvcPABC- |
| | Compound 139 | Compound 139 | Compound | MMAE |
| | DAR 8 at 6 | DAR 4 at 6 | 139 DAR 4 | DAR 4 at 6 |
| | mg/kg | mg/kg | at 12 mg/kg | mg/kg |
| LU5213 | 31% | −10% | | |
| LU5245 | 52% | 18% | | |
| LU6802 | 95% | 45% | 73% | 50% |
| LU6904 | 64% | 44% | 29% | 51% |
| LU11692 | 30% | 57% | | 6% |
| LU11796 | 92% | 87% | | 97% |
| LU11870 | 57% | 49% | | 32% |
| LU11876 | 60% | 54% | | 86% |

The data provided in Table 37.9 demonstrates that v38591-MC-GGFG-AM-Compound 139 has anti-tumor activity in NSCLC xenograft models.

For all NSCLC patient-derived xenograft (PDX) models, research level immunohistochemistry (IHC) assessment on same study tissue will be performed according to the method described in Example 35.

### Example 38: In Vivo Activity of Antibody-Drug Conjugates in Patient-Derived Models of Endometrial Cancer

In vivo anti-tumor activity of ADCs of humanized variant v38591 was assessed in four patient-derived (PDX) models of endometrial cancer, UT14026, UT5318, UT5326 and UT5321. An ADC of reference antibody v18993 (lifastuzumab) conjugated to MCvcPABC-MMAE at DAR4 was also tested as a comparator in some models. These studies were carried out as described below.

For all endometrial cancer PDX models, anti-tumor activity was determined by % tumor growth inhibition (% TGI) calculated as [(1−TVₜᵣₑₐₜₘₑₙₜ/TV_{vehicle})×100] at Day 28, or at the closest evaluable time point, as summarized in Table 38.5.

For the NSCLC cancer PDX model UT14026, tumor fragments were implanted subcutaneously into NOD/SCID mice. When mean tumor volume reached ~140 mm³ the animals were assigned to groups (n=3 per group) and treated on study day 0 with a single IV dose of test articles as shown in Table 38.1.

TABLE 38.1
Treatment groups for UT14026 endometrial cancer model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v38591-MC-GGFG-AM-Compound 139 | 4 | 12 |
| 5 | v18993 (lifastuzumab)-MCvcPABC-MMAE | 4 | 6 |

For the NSCLC PDX model UT5318, tumor fragments were implanted subcutaneously into female NOD/SCID mice. When mean tumor volume reached ~180 mm³ the animals were assigned to groups (n=3 per group) and treated on study day 0 with a single IV dose of test articles as shown in Table 38.2.

TABLE 38.2
Treatment groups for UT5318 endometrial cancer Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v38591-MC-GGFG-AM-Compound 139 | 4 | 12 |
| 5 | v18993 (lifastuzumab)-MCvcPABC-MMAE | 4 | 6 |

For the NSCLC PDX model UT5326, tumor fragments were implanted subcutaneously into female BALB/c nude mice. When mean tumor volume reached ~170 mm³ the animals were assigned to groups (n=3 per group) and treated on study day 0 with a single IV dose of test articles as shown in Table 38.3.

TABLE 38.3
Treatment groups for UT5326 endometrial cancer Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v38591-MC-GGFG-AM-Compound 139 | 4 | 12 |
| 5 | v18993 (lifastuzumab)-MCvcPABC-MMAE | 4 | 6 |

For the NSCLC PDX model UT5321, tumor fragments were implanted subcutaneously into female BALB/c nude mice. When mean tumor volume reached ~155 mm³ the animals were assigned to groups (n=3 per group) and treated on study day 0 with a single IV dose of test articles as shown in Table 38.4.

TABLE 38.4
Treatment groups for UT5321 endometrial cancer Model

| Group | Treatment | DAR | Dose (mg/kg) |
|---|---|---|---|
| 1 | Vehicle | | 0 |
| 2 | v38591-MC-GGFG-AM-Compound 139 | 8 | 6 |
| 3 | v38591-MC-GGFG-AM-Compound 139 | 4 | 6 |
| 4 | v38591-MC-GGFG-AM-Compound 139 | 4 | 12 |
| 5 | v18993 (lifastuzumab)-MCvcPABC-MMAE | 4 | 6 |

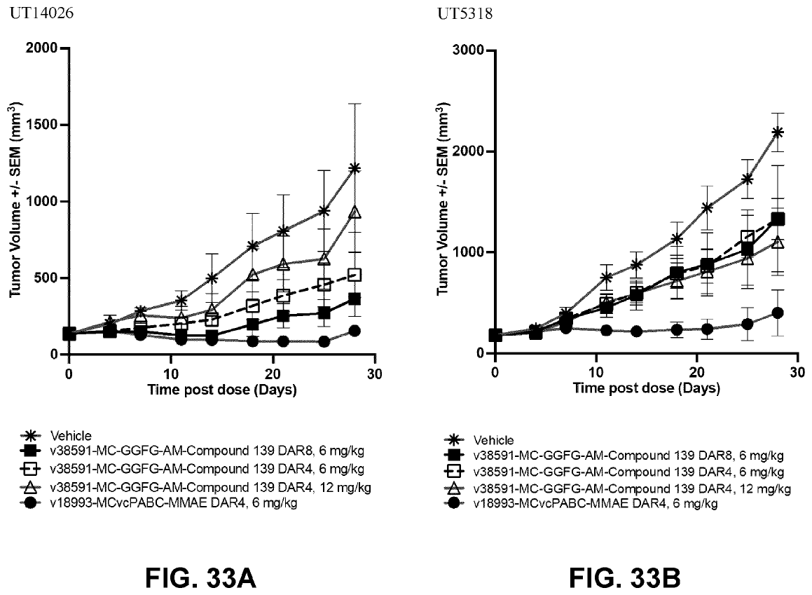

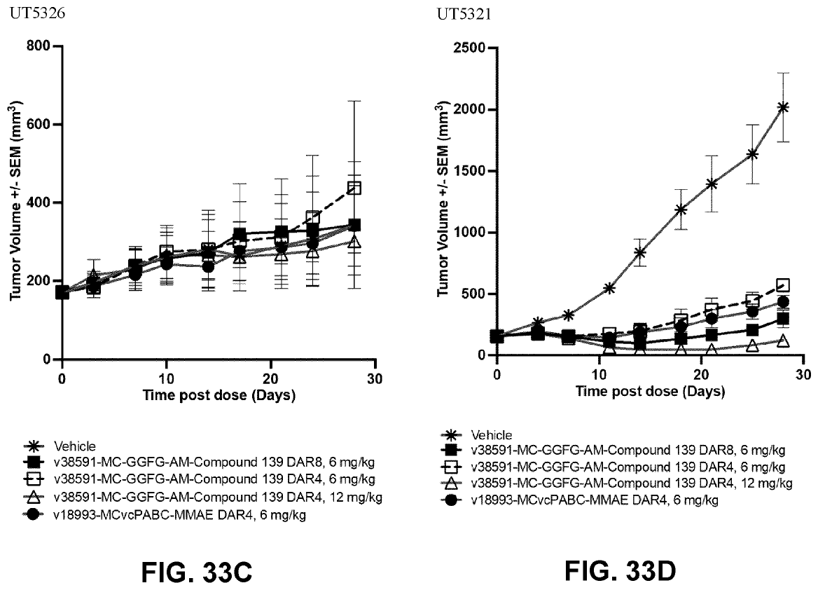

The results for the UT14026 study are shown in FIG. 33A. At 6 mg/kg, v38591-MC-GGFG-AM-Compound 139 at DAR8 resulted in a strong inhibition of tumour growth. At a DAR of 4, v38591-MC-GGFG-AM-Compound 139 resulted in a moderate inhibition of tumour growth at 6 mg/kg and at 12 mg/kg. For comparison, v18993 (lifastuzumab)-MCvcPABC-MMAE resulted in a strong inhibition of tumour growth at 6 mg/kg.

The results for the UT5318 study are shown in FIG. 33B. At 6 mg/kg, v38591-MC-GGFG-AM-Compound 139 resulted in a moderate inhibition of tumour growth at a DAR of 4 or 8. At 12 mg/kg, v38591-MC-GGFG-AM-Compound 139 resulted in a moderate inhibition of tumour growth at a DAR of 4. For comparison, v18993 (lifastuzumab)-MCvcPABC-MMAE resulted in a strong inhibition of tumour growth at 6 mg/kg.

The results for the UT5326 study are shown in FIG. 33C. At 6 mg/kg, v38591-MC-GGFG-AM-Compound 139 did not result in substantial inhibition of tumour growth at a DAR of 4 or 8. At 12 mg/kg, v38591-MC-GGFG-AM-Compound 139 did not result in substantial inhibition of tumour growth at a DAR of 4. For comparison, v18993 (lifastuzumab)-MCvcPABC-MMAE did not result in a substantial inhibition of tumour growth at 6 mg/kg.

The results for the UT5321 study are shown in FIG. 33D. At a DAR4, v38591-MC-GGFG-AM-Compound 139 resulted in a strong inhibition of tumour growth at a 6 mg/kg and 12 mg/kg. At a DAR8, v38591-MC-GGFG-AM-Compound 139 resulted in a strong inhibition of tumour growth at 6 mg/kg. For comparison, v18993 (lifastuzumab)-MCvcPABC-MMAE resulted in strong inhibition of tumour growth at 6 mg/kg.

TABLE 38.5
Summary of anti-tumor activity (% tumor growth inhibition)
in endometrial cancer PDX models

| | v38591-MC- | v38591-MC- | v38591-MC- | v18993- |
|---|---|---|---|---|
| | GGFG-AM- | GGFG-AM- | GGFG-AM- | MCvcPABC- |
| | Compound 139 | Compound 139 | Compound | MMAE |
| | DAR 8 at 6 | DAR 4 at 6 | 139 DAR 4 | DAR 4 at 6 |
| | mg/kg | mg/kg | at 12 mg/kg | mg/kg |
| UT14026 | 70% | 57% | 23% | 87% |
| UT5318 | 39% | 39% | 50% | 82% |
| UT5326 | 0 | −27% | 12% | 1% |
| UT5321 | 85% | 72% | 94% | 78% |

The data provided in Table 38.5 demonstrates that v38591-MC-GGFG-AM-Compound 139 has anti-tumor activity in endometrial cancer xenograft models.

For all endometrial cancer patient-derived xenograft (PDX) models, research level immunohistochemistry (IHC) assessment on same study tissue was performed by staining with a commercial anti-NaPi2b antibody (Clone D6W2G, Rabbit mAb #42299, Cell Signaling Technology). NaPi2b H-scores will be determined by a pathologist for all PDX models. H-scores will be calculated on a scale from 0-300 using the formula: H-score=(0×P₀)+(1×P₁)+(2×P₂)+(3×P₃), where P₀, P₁, P₂, and P₃ represent the percentage of cells with absent, weak, moderate or strong staining, respectively.

### Example 39: Preparation of Anti-NaPi2B Antibody v40502 with L234A, L235A and D265S Modifications

As there may be benefits of having an Fe knockout in the anti-NaPi2b antibody construct of the ADCs described herein, compared to an anti-NaPi2b antibody construct with a wild-type Fc, a modified anti-NaPi2b antibody construct based on the sequence of v38591 was constructed, having mutations at positions L234A, L235A and D265S (EU numbering) of the Fc region. This modified construct is referred to herein as v40502, or v40502 (LALADS) and was constructed as follows.

Humanized VH and VL sequences of v38591 (corresponding to the VH and VL sequences of v29456 provided in Examples 7 and 8 as SEQ ID NO:24 (VH) and SEQ ID NO:29 (VL)) were used to construct v40502 (LALADS), such that the coding sequence of the antibody variable regions were cloned in frame into a human IgG1 expression vector (with human IgG1 constant region starting with alanine 118 according to Kabat numbering and bearing L234A, L235A and D265S mutations (EU numbering) [SEQ ID NO:64]; see Table 39.1) or a human C kappa expression vector (with human C kappa constant region starting at arginine 108 according to Kabat numbering: [SEQ ID NO:65]; see Table 39.1), both expression vectors based on pTT5. The v40502 (LALADS) variant includes C-terminal lysines in both heavy chains, similar to the v38591 antibody; these C-terminal lysines are cleaved from the majority of the expressed antibody once it is secreted from the cell it is produced in.

Two identical full-length heavy chains and two identical kappa light chains as described above were assembled into full-size antibody (FSA) format.

TABLE 39.1
Human Constant Heavy (LALADS) and Light Chain Sequences

| | | SEQ ID |
|---|---|---|
| | Sequence | NO |
| CH1-hinge-CH2- | ASTKGPSVFPLAPSSKSTSGGTAALGCLVKDYFPEPVTVSWNSG | 64 |
| CH3 | ALTSGVHTFPAVLQSSGLYSLSSVVTVPSSSLGTQTYICNVNHK | |
| (IGHG1*01) | PSNTKVDKKVEPKSCDKTHTCPPCPAPEAAGGPSVFLFPPKPKD | |
| with L234A | TLMISRTPEVTCVVVSVSHEDPEVKFNWYVDGVEVHNAKTKP | |
| L235A D265S | REEQYNSTYRVVSVLTVLHQDWLNGKEYKCKVSNKALPAPIE | |
| | KTISKAKGQPREPQVYTLPPSRDELTKNQVSLTCLVKGFYPSDI | |
| | AVEWESNGQPENNYKTTPPVLDSDGSFFLYSKLTVDKSRWQQ | |
| | GNVFSCSVMHEALHNHYTQKSLSLSPGK | |
| CL (kappa) | RTVAAPSVFIFPPSDEQLKSGTASVVCLLNNFYPREAKVQWKV | 65 |
| (IGKC*01) | DNALQSGNSQESVTEQDSKDSTYSLSSTLTLSKADYEKHKVYA | |
| | CEVTHQGLSSPVTKSFNRGEC | |

TABLE 39.2
HC and LC Sequences of v40502 (LALADS)

| v40502 | | SEQ ID |
|---|---|---|
| (LALADS) | Sequence | NO |
| HC | QVQLVQSGAEVKKPGASVKVSCKASGFTFTSYNVHWVRQAPG | 66 |
| | QGLEWMGAISPGNGDLSYAQKFQGRVTMTRDTSTSTVYMELSS | |
| | LRSEDTAVYYCARGRTGMGAVDYWGQGTLVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCDKTHTCPPCPAPEAAGGPSVFLFPPKPKDTLMISRTPE | |
| | VTCVVVSVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |
| LC | DVQMTQSPSSLSASVGDRVTITCRASQDVYSYLNWYQQKPGKA | 67 |
| | VKLLIYYTSRLQSGVPSRFSGSGSGTDYTLTISSLQPEDFATYYC | |
| | QQYNIFPWTFGQGTKLEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| HC (without C- | QVQLVQSGAEVKKPGASVKVSCKASGFTFTSYNVHWVRQAPG | 68 |
| terminal lysine) | QGLEWMGAISPGNGDLSYAQKFQGRVTMTRDTSTSTVYMELSS | |
| | LRSEDTAVYYCARGRTGMGAVDYWGQGTLVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCDKTHTCPPCPAPEAAGGPSVFLFPPKPKDTLMISRTPE | |
| | VTCVVVSVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPG | |

### 39.1 Expression and Purification of v40502 (LALADS)

The antibody construct v40502 (LALADS) was prepared as follows in 2 batches (400 ml each).

ExpiCHO™ cells were cultured at 37° C. in ExpiCHO™ expression medium (Thermo Fisher, Waltham, MA) on an orbital shaker rotating at 120 rpm in a humidified atmosphere of 800 CO₂. 400 mL expression volumes were used. Each 1 mL of cells at a density of 6×10⁶ cells/mL was transfected with a total of 0.8 g DNA. Prior to transfection the DNA was diluted in 76.8 L OptiPRO™ SFM (Thermo Fisher, Waltham, MA), after which 3.2 L of ExpiFectamine™ CHO reagent (Thermo Fisher, Waltham, MA) was directly added to make a total volume of 80 μL. After incubation for 1-5 minutes, the DNA-ExpiFectamine™ CHO Reagent complex was added to the cell culture (80 uL complex per 1 mL of cell culture) then incubated in a 120 rpm shaking incubator at 37° C. and 8% CO₂. Following incubation at 37° C. for 18-22 hours, 6 L of ExpiCHO™ Enhancer and 240 L of ExpiCHO™ Feed (Thermo Fisher, Waltham, MA) were added per 1 mL of culture. Cells were maintained in culture at 37° C. for a total of 8 days, after which each culture was harvested by transferring into appropriately sized centrifuge tubes and centrifuging at 4200 rpm for 15 minutes. Supernatants were filtered using a 0.2 μm polyethersulfone membrane (Thermo Fisher, Waltham, MA), then analyzed by non-reducing SDS-PAGE and Octet (ForteBio).

Protein purification was performed in either batch mode or with the use of an AKTA™ Pure purification system. In batch mode, supernatants from transient transfections were applied to slurries containing 50% MabSelect SuRe™ resin (Cytiva, Marlborough, MA) and incubated at room temperature for 1 hr on an orbital shaker at 150 rpm. The slurries were transferred into chromatography columns and supernatants were allowed to flow through while resins remained in the column. The resins were then washed with at least 5 Bed Volumes (BV) of resin Equilibration buffer (PBS). To elute the targeted proteins, 2.5 BV of Elution Buffer (100 mM sodium citrate buffer pH 3.5) was added to the columns and collected. Elutions were then neutralized by adding 20% (v/v) 1 M Tris pH 9 to reach a final pH of 7.0. In AKTA™ Pure purification mode, supernatants from transient transfections were loaded onto HiTrap MabSelect SuRE LX columns (Cytiva, Marlborough, MA) that were pre-equilibrated with 5 Column Volume (CV) of PBS. After the proteins were captured, the columns were then washed with 10 CV of PBS. The captured proteins were eluted with 5 CV of Elution Buffer (100 mM sodium citrate buffer pH 3.5) in fractions. Pooled fractions were neutralized with 20% (v/v) if 1 M Tris pH 9. Samples were then buffer exchanged into PBS pH 7.4. The protein content of samples was determined by 280 nm absorbance measurement using a Nanodrop™

The purity of protein samples was assessed by non-reducing and reducing LabChip™ CE-SDS. LabChip™ GXII Touch (Perkin Elmer, Waltham, MA). Analysis was carried out according to Protein Express Assay User Guide (PerkinElmer, Waltham, MA), with the following modifications. Samples at a concentration range of 5-2000 ng/μL were added to separate wells in 96 well plates (#MSP9631, BioRad, Hercules, CA) along with 7 μL of HT Protein Express Sample Buffer (#CLS920003, Perkin Elmer) and denatured at 90° C. for 5 mins. The LabChip™ instrument was operated using the LabChip™ HT Protein Express Chip (Perkin Elmer #760528) with HT Protein Express 200 assay setting.

Final yields from two production batches were ~57 and 64 mg (~142-160 mg/L of culture). The purified antibody displayed Caliper profiles reflective of expected antibody composition reflecting a single species corresponding to full-size antibody (NR Caliper) and intact heavy and light chains for all antibodies (R Caliper) (data not shown). 39.2 Quality Assessment of modified h12A10 antibody

Species homogeneity of the antibody was assessed by UPLC-SEC after protein-A purification (final purification step).

Samples were analyzed as follows: UPLC-SEC was performed using an Agilent Technologies AdvanceBio SEC300A SEC column (7.8×150 mm, 1.7 m particles) (Agilent Technologies, Santa Clara, California) set to 25° C. and mounted on an Agilent Technologies 1260 infinity II system with a DAD detector. Run times consisted of 7 min and a total volume per injection of 5 μL with a running buffer of 200 mM K₃PO₄, 200 mM KCl, pH 7. Elution was monitored by UV absorbance in the range 190-400 nm, and chromatograms were extracted at 280 nm. Peak integration was performed using OpenLAB™ CDS ChemStation™ software. The profile reflected high species homogeneity (data not shown).

### Example 40: Assessment of v40502 (LALADS) Anti-NaPi2B Antibody Binding to FcγR

SPR analysis was carried out to characterize binding of the v40502 (LALADS) anti-NaPi2b antibody to human Fc gamma receptors (FcγR or FcγRs) reagents using a Cytiva Biacore™ T200 instrument. The binding characteristics of this antibody were compared to the wildtype (v38591) and commercial Trastuzumab (Roche Diagnostics).

### Surface Plasmon Resonance (SPR)

Binding kinetics was carried out on a Series S Protein A Chip (Cytiva©, 29127556) with PBS-T (PBS+0.05% (v/v) Tween 20, pH7.4) running buffer at a temperature of 25° C. Approximately 400-600 RUs of each antibody (2 μg/mL) were captured on the protein A surface on flow cell 2, 3 and 4 by injecting at 10 μL/min for 60 s. Flow cell 1 was used as a reference surface. Six types of human FcγRs were tested, including a commercial FcγRI CD64 (Sino Biological Inc. 10256-H08H) and five in-house produced FcγRs (Table 40.1). Five concentrations of a 3-fold dilution series of FcγRs were prepared starting at 12 μM, 5 μM, or 30 nM, depending on the FcγR proteins. Each in-house FcγR with five concentrations were flowed as analyte using single-cycle kinetics strategy. The flow rate of FcγRs was 50 L/min with contact time of 40 sec and dissociation time of 120 sec. For the high affinity FcγRI CD64, multicycle kinetics were applied. The flow rate of the analyte was 100 L/min with contact time of 100 sec and dissociation time of 650 sec. Blank buffer control was injected using the same flow rate and contact time for each cycle. Surface regeneration was accomplished using 10 mM glycine-HCl, pH 1.5 (Cytiva©, BR100354) for 30 sec with a flow rate of 50 μL/min after each cycle. Binding constants and the maximum observed binding signal (Rmax) were determined using the Biacore™ T200 Evaluation software (version 3.0; GE Healthcare). For single-cycle kinetics, blank-subtracted sensorgrams were analyzed using steady state affinity fitting model. For multicycle kinetics, results were fit to the 1:1 Langmuir binding model. Binding percentage was calculated from observed Rmax and theoretical Rmax (Rmax/Rmax_ₜₕₑₒ), based on antibody capture density.

Binding results of SPR analysis are shown in Table 40.1. Six FcγRs reagents were tested, covering type I, II and III human FcγRs. Rmax values and binding percentage indicated that the LALADS mutations fully abrogated all binding to human FcγRs with nearly no binding detected for v40502 (LALADS). Trastuzumab and the wildtype v38591 without the Fc LALADS mutation showed nearly full binding to the tested FcγRs.

TABLE 40.1
Binding of test antibodies to human FcγRs by SPR

| | FcγR Sample | | Rmax | | Binding |
|---|---|---|---|---|---|
| Ligand | Variant | FcγR type | (RU) | K_{D} (M) | percentage |
| Trastuzumab | Sino | FcγR I | 232.6 | 4.16E−11 | 90.2% |
| v38591Wild- | Biological | CD64 | 172.7 | 5.85E−11 | 91.2% |
| type | Inc. | | | | |
| v40502 | | | 0.4 | 1.72E−04 | 0.2% |
| LALADS | | | | | |
| Trastuzumab | v5772 | FcγR IIIA | 120.7 | 1.88E−06 | 90.9% |
| v38591Wild- | | F159 | 90.2 | 1.13E−06 | 92.5% |
| type | | CD16aF | | | |
| v40502 | | | NB | NB | NB |
| LALADS | | | | | |
| Trastuzumab | v5773 | FcγR IIIA | 134.1 | 7.08E−07 | 101.9% |
| v38591Wild- | | V159 | 92.9 | 4.23E−07 | 96.1% |
| type | | CD16aV | | | |
| v40502 | | | 1.5 | 4.47E−07 | 1.8% |
| LALADS | | | | | |
| Trastuzumab | v5774 | FcγR IIA | 124.3 | 8.29E−07 | 82.7% |
| v38591Wild- | | H131 | 88.4 | 7.99E−07 | 80.0% |
| type | | CD32aH | | | |
| v40502 | | | 1.2 | 2.49E−06 | 1.3% |
| LALADS | | | | | |
| Trastuzumab | v5775 | FcγR IIA | 133.9 | 1.21E−06 | 99.9% |
| v38591Wild- | | R131 | 96.8 | 1.06E−06 | 98.2% |
| type | | CD32aR | | | |
| v40502 | | | 0.4 | 2.77E−06 | 0.5% |
| LALADS | | | | | |
| Trastuzumab | v5777 | FcγR IIB | 133.7 | 5.81E−06 | 101.2% |
| v38591Wild- | | Y160 | 100.3 | 4.98E−06 | 103.2% |
| type | | CD16aV | | | |
| v40502 | | | 1.0 | 2.87E−08 | 1.2% |
| LALADS | | | | | |

Rmax—the maximum observed binding signal;
K_{D}—Equilibrium dissociation constant;
RU—Response units;
M—Molar;
NB—No binding

### Example 41: Developability Assessment of LALADS Anti-NaPi2B Antibody

The isoelectric point, propensity for self-aggregation, and non-specific binding of LALADS anti-NaPi2b antibody v40502 was determined and compared to its wildtype v38591 as an assessment of developability. The isoelectric point was measured by capillary isoelectric focusing (cIEF), the propensity for self-aggregation was measured by affinity-capture self-interaction nanoparticle spectroscopy (AC-SINS) and non-specific binding was measured by NS-ELISA, as described below.

Capillary Isoelectric Focusing (cIEF)

cIEF was conducted using Maurice C. system, System Suitability Kit and Method Development Kit (Protein Simple©). System suitability standard, fluorescence calibration standard, cartridge and samples were prepared according to vendor's recommendations. The capillary was automatically calibrated with a fluorescence standard preconditioned with Maurice cIEF System Suitability Kit to ensure the capillary was functioning properly. The antibody samples were diluted to a concentration of 0.5 mg/mL in a final volume of 40 μL in Gibco™ Distilled Water, and mixed Maurice cIEF Method Development Kit Samples. The samples were then vortexed, centrifuged and the supernatant pipetted into individual wells of a 96-well plate. All electropherograms were detected with UV absorbance at 280 nm and protein native fluorescence. All data analyses were performed using vendor software Compass for iCE (ProteinSimple©). The Compass software aligns each electropherogram using the pI markers so that the x-axis is displayed as a normalized pI for each injection.

The pI values were determined for the main isoform for the variants. The measured pI was compared to in-house made Trastuzumab and anti-NaPi2b antibody v38951 having a wild-type Fc region. The measured pI of LALADS anti-NaPi2b antibody was expected to be slightly higher its wildtype.

### Affinity-Capture Self-Interaction Nanoparticle Spectroscopy (AC-SINS) Assay

AC-SINS method was carried out in a 384-well plate format (Corning® #3702). Initially, 20 nm gold nanoparticles (Ted Pella, Inc., #15705) washed with 0.22 μm filtered Gibco™ Distilled Water were coated with a mixture of capture antibody-80% AffiniPure Goat Anti-Human IgG (H+L) (Jackson ImmunoResearch Laboratories© #109-005-088), and the non-capture antibody—20% ChromPure Goat IgG, whole molecule (Jackson ImmunoResearch Laboratories© #005-000-003), that were initially buffer exchanged into 20 mM sodium acetate pH 4.3 and diluted to 0.4 mg/mL. The mixture of gold nanoparticles, capture antibody and non-capture antibody was incubated in the dark for 18 h at room temperature. Sites unoccupied on the gold nanoparticles were blocked with 1 μM thiolated polyethylene glycol (2 kD) in 20 mM sodium acetate, pH 4.3 to a final concentration of 0.1 μM, followed by 1 h incubation at room temperature. The coated nanoparticles were then concentrated by centrifugation at 21,000×g for 7 min, at 8° C. 95% of the supernatant was removed and the gold pellet was resuspended in the remaining buffer. 5 μL of concentrated nanoparticles were added to 45 μL of antibody at 0.05 mg/mL in Gibco™ PBS pH 7.4 in a 384-well plate. The coated nanoparticles were incubated with the antibody of interest for 4 h at room temperature in the dark. The absorbance was scanned from 450-700 nm at 1 nm increments, and a Microsoft Excel macro was used to identify the max absorbance, smooth the data, and fit the data using a second-order polynomial.

The Δlambda (nm) was calculated based on the smoothed max absorbance of the average blank (PBS alone) subtracted from the smoothed max absorbance of the antibody sample to determine the antibody AC-SINS score. Antibody-antibody interactions directly correlate with the shift in maximum absorbance wavelength of gold nanoparticles coated with the antibody of interest. The cutoff of Δlambda 10 nm was set as high self-aggregation propensity of the antibody, based on the literature.

### Non-Specific (NS)-ELISA

NS-ELISA was used to measure the propensity of the antibodies to bind to a range of biomolecules to emulate the undesirable non-specific interactions to biological matrices in vivo as described below.

NS-ELISA was carried out in a Corning® 96-well EIA/RIA Easy Wash™ Clear Flat Bottom Polystyrene High Bind Microplate coated overnight at 4° C. with 50 μL of Heparin (Sigma, H3149) diluted with 50 mM sodium carbonate pH 9.6 to a final concentration of 250 μg/mL. The plate was incubated for 2 days at room temperature, wells that were coated with heparin were not covered to allow air dry. Insulin (Sigma-Aldrich®, 19278) and KLH (Sigma-Aldrich®, H8283) were each diluted with 50 mM sodium carbonate pH 9.6 to a final concentration of 5 μg/mL. ssDNA (Sigma-Aldrich®, D8899) and dsDNA (Sigma-Aldrich®, D4553) was diluted with Gibco™ PBS pH7.4 to a final concentration of 10 μg/mL. 50 μL each of insulin, KLH, dsDNA and ssDNA were added to a 96 well plate, followed by the incubation at 37° C. for 2 h. The coating materials were removed, and the plate was blocked with 200 μL of Gibco™ PBS pH7.4, 0.1% Tween®20, and incubated for 1 h at room temperature with shaking at 200 rpm. The plate was washed 3 times with Gibco™ PBS pH7.4, 0.1% Tween 20. 50 μL of each mAb at 100 nM (15 mg/mL) in Gibco™ PBS pH 7.4, 0.1% Tween®20 was added in duplicate to the wells and incubated for 1 h at room temperature with shaking at 200 rpm. Plates were washed three times with Gibco™ PBS pH7.4, 0.1% Tween 20, and 50 μL of 50 ng/mL anti-human IgG HRP (Thermofisher Scientific©, H10307) was added to each well. Plates were incubated for 1 h at room temperature, with shaking at 200 rpm. The plate was washed three times with Gibco™ PBS pH7.4, 0.1% Tween 20, and 100 μL of TMB substrate (Cell Signaling Technology©, 7004P6) added to each well. Reactions were stopped after approximately 10 minutes by adding 100 μL of 1 M HCl to each well, and absorbance was read at 450 nm.

Binding scores were calculated as the ratio of the ELISA signal of the antibody to the signal of a well containing buffer instead of the primary antibody. The cutoffs considered for each binding molecule (ssDNA. KLH, Insulin, dsDNA and Heparin) were internally calculated, based on the average of Zymeworks Inc. produced antibodies and antibodies benchmarks published in the literature.

The results of all three assays are shown in Table 41.1. For cIEF, the LALADS variant showed a measured pI of 9.229, which is slightly higher but close to pI of the wildtype (9.160) and Trastuzumab (8.902). In AC-SINs and NS-ELISA assays, a score higher than the cutoff was taken to indicate potentially less desirable biophysical characteristics. AC-SINs and NS-ELISA did not identify any potential issues for any of the tested antibodies.

TABLE 41.1
Developability assessment results for anti-NaPi2b antibodies

| | | | AC-SINS | | | | | |
|---|---|---|---|---|---|---|---|---|
| | | cIEF | Δlambda | NS-ELISA (Binding score) | | | | |
| Sample | Variant | pI | (nm) | ssDNA | KLH | Insulin | dsDNA | Heparin |
| LALADS | v40502 | 9.229 | 1.50 | 0.92 | 1.02 | 1.07 | 0.99 | 0.90 |
| Wildtype | v38591 | 9.160 | 3.50 | 0.80 | 0.97 | 1.99 | 0.97 | 1.17 |
| Trastuzumab | v17078 | 8.902 | 0.00 | 0.72 | 1.03 | 1.08 | 1.14 | 0.87 |

Assay cutoffs are shown as following,
AC-SINS: Δlambda < 10 nm;
NS-ELISA: ssDNA < 5.5, KLH < 4.5, Insulin < 2.5, dsDNA < 4.5, Heparin < 3.0.

### Example 42: Assessment of Stability of v40502 (LALADS) Antibody

To investigate the stability of variant 40502, the following studies were performed: 4° C. and 40° C. thermal stability, freeze and thaw, photo stability, and low pH stability, in which samples were characterized at specific timepoints by UPLC-SEC and RPLC-MS.

For thermal stability study, variant 40502 was buffer exchanged into either 20 mM Histidine pH 5.0 with 6% w/v sucrose (His 5Su) or 20 mM Sodium Succinate pH 4.5 with 6% w/v sucrose (Succ5Su) and incubated at 1 mg/ml at either 4° C. or 40° C., protected from light. Samples were evaluated by uPLC-SEC and RPLC-MS at timepoints 0, 7 and 13 days. The 40° C. samples underwent an additional incubation of 15 min at 60° C.

For low pH stability study, variant 40502 was buffer exchanged into 20 mM sodium citrate pH 3.5 and incubated at 1 mg/ml at room temperature. Sample was evaluated by uPLC-SEC and RPLC-MS at timepoint 3 hours.

For photo stability study, variant 40502 was buffer exchanged into either His 5Su or Succ5Su and incubated at 1 mg/ml exposed directly to the light of a biological safety cabinet, at room temperature, and evaluated by uPLC-SEC and RPLC-MS at timepoint 7 days.

For the freeze and thaw study, variant 40502 was buffer exchanged into either His 5Su or Succ5Su and transferred to −20° C. After 16 hours at −20° C., the sample underwent 4 cycles of transfer to room temperature for 6 min, followed by transfer to −20° C. for 10 minutes. After the final room temperature transfer, the protein was evaluated by uPLC-SEC and RPLC-MS.

For RPLC-MS, the photo, freeze-thaw, and low pH stability samples were diluted 1:1 v:v with PBS. Thermal stability samples were not diluted. Then, 10 μL samples were deglycosylated with 1 μg of recombinant EndoS endoglycosidase per 10 μg of antibody for one hour at room temperature. Also, thermal stability samples were reduced with 1 μl of 0.5 M TCEP for one hour after the intact RPLC-MS runs were finished and re-injected for RPLC-MS analysis. For RPLC-MS analysis, 1 μL of sample were injected into a Waters™ BioSuite Phenyl Column, 1000 Å, 10 μm, 4.6 mm×75 mm using an Agilent 1290 Infinity II Liquid Chromatography System coupled with Agilent 6545 Quadrupole Time of Flight (Q-TOF) with a column temperature of 70° C. and a flow rate of 0.3 ml/min. Mobile phases consisted of A: LC-MS grade water with 0.1% v/v formic acid, 0.025 v/v trifluoroacetic acid and 10% v/v isopropyl alcohol in, and B: acetonitrile with 0.1% v/v formic acid and 10% v/v isopropyl alcohol. The column was pre-equilibrated in 10% mobile phase B before injection. Then, a 5 min 10 to 60% mobile phase B gradient was applied, followed by a 2 min 60 to 90% mobile phase B gradient and a column wash of 2 min at 99% mobile phase B. The column was re-equilibrated to 10% mobile phase B for 2 minutes between runs. ESI (electro spray ionization) was performed in a Dual AJS source in positive mode with 5 kV of VCap voltage, 2 kV Nozzle voltage, 170 V fragmentor voltage, gas temperature of 300° C., gas flow of 13 L/min, nebulizer pressure of 45 psig, sheath gas temperature of 400° C. and a sheath gas flow of 12 L/min. Data format was continuum with analyser set in sensitivity mode, with a m/z range from 500 to 7000 with a scan rate of 1 spectra per sec.

Peak integration, mass spectrum deconvolution and mass assignments were performed in Protein Metrics Byos® v4.6 using a deconvolution window of 20000-163000 Da with an m/z range of 1000-4000. For all time points, the highest intensity deconvoluted mass was assigned as the reference mass of variant 40502 or a corresponding N-glycan adduct of the reference mass. The reference mass was defined as the average mass of variant 40502 with two 2-acetamido-2-deoxy-beta-D-glucopyranose-(1-4)-[alpha-L-fucopyranose-(1-6)] stubs arising from EndoS activity on N-glycans, 16 disulfide bonds and the formation of pyroglutamic acid, if applicable, at the N-terminus. Mass assignments had a mass tolerance of 10 Da. Other variant 40502 adducts assigned based on mass shifts relative to the reference mass were: variant 40502 reference mass with the loss of one fucose unit, variant 40502 reference mass with the addition of a hexose unit. N-glycan adducts relative to the reference mass of variant 40502 without 2-acetamido-2-deoxy-beta-D-glucopyranose-(1-4)-[alpha-L-fucopyranose-(1-6)] stubs were also identified.

Analytical SEC (Size Exclusion Chromatography) was performed using an Agilent Infinity 111290 HPLC with Advance Bio SEC column (300 Å, 2.7 μm, 7.8×150 mm) equilibrated with 5 column volumes of Mobile Phase A (200 mM KPO4, 200 mM KCl, pH 7.0) at room temperature. 5 μl of incubated sample was injected and eluted isostatically for 7 mins at 1 mL/min with absorbance monitored at A280. Chromatograms were exported and integrated in PMI Byos v4.6 to provide complete, baseline-to-baseline integration of each peak. The peak corresponding to the major component for IgG (approximate peak apex time 3.5 min, integration window from 3.1 to 4.2 min) was reported as the monomer based on the SEC profile of control trastuzumab. A time window from 2 to 3.1 min was designated as high molecular weight species (HMWS), and a time window from 4.2 to 5.0 min was designated as low molecular weight species (LMWS), excluding solvent peaks (over 5.0 min).

The uPLC-SEC results are shown in Table 42.1. The protein was stable at all conditions as evaluated by uPLC-SEC, based on the similarity of monomer % relative to day 0 controls (<2% decrease). RPLC-MS results are shown in Table 42.2. The protein was stable at most conditions as evaluated by RPLC-MS, based on the similarity of desired proteoform % relative to day 0 controls (<10% decrease).

TABLE 42.1
Stability studies, uPLC-SEC.

| | | % total UV |
|---|---|---|
| | % | 280 nm peak |
| | monomer | area relative |
| | by uPLC- | to day 0 |
| Sample name | SEC | timepoint |
| His5Su day 0 control | 95.2% | na |
| His5Su 4° C. Day 7 | 95.3% | 95% |
| His5Su 4° C. Day 13 | 95.4% | 92% |
| His5Su 40° C. Day 7 | 96.8% | 96% |
| His5Su 40° C. Day 13 | 97.0% | 108% |
| His5Su photo stability 7 days | 95.7% | 97% |
| His5Su freeze thaw | 95.8% | 90% |
| Succ5Su day 0 control | 94.4% | na |
| Succ5 Su 4° C. Day 7 | 95.2% | 100% |
| Succ5Su 4° C. Day 13 | 95.1% | 103% |
| Succ5Su 40° C. Day 7 | 96.6% | 102% |
| Succ5Su 40° C. Day 13 | 96.1% | 107% |
| Succ5 Su photo stability 7 days | 97.1% | 98% |
| Succ5 Su freeze thaw | 94.8% | 106% |
| 20 mM Na Citrate pH 3.5 3 h | 93.2% | 118% |
| Trastuzumab, neat | 98.2% | na |

TABLE 42.2
Stability studies, RPLC-MS.

| | Desired |
|---|---|
| | proteoforms |
| Condition | % |
| His5Su day 0 Ctrl | 89% |
| His5Su 4° C. day 7 | 93% |
| His5Su 4° C. day 13 | 94% |
| His5Su 40° C. day 7 | 91% |
| His5Su 40° C. day 13 | 92% |
| H5 Su photostability RT 7 d | 90% |
| H5Su 5x freeze thaw | 89% |
| Succ5 Su day 0 Ctrl | 92% |
| Succ5Su 4° C. day 7 | 92% |
| Succ5Su 4° C. day 13 | 94% |
| Succ5Su 40° C. day 7 | 92% |
| Succ5Su 40° C. day 13 | 90% |
| Succ5 Su photostability RT 7 d | 92% |
| Succ5Su 5x freeze thaw | 83% |
| NaCitrate pH 3.5 3 h intact | 87% |

### Example 43: Preparation of Antibody-Drug Conjugates of v40502 (LALADS)

Antibody-drug conjugates shown in Table 43.1 were prepared. Exemplary protocols are provided below.

TABLE 43.1

| Variant | Antibody | Drug-Linker¹ | DAR |
|---|---|---|---|
| v40502 | humanized | MC-GGFG-AM-Compound 139 | 4 |
| v38591 | humanized | MC-GGFG-AM-Compound 139 | 4 |
| | | MC-GGFG-AM-Compound 139 | 8 |

v40502-MC-GGFG-AM-Compound 139 DAR4: A solution (5.07 mL) of the v40502 variant (29 mg) in PBS, pH 7.4 was diluted in PBS, pH 7.4 (0.53 mL) and reduced by addition of 5 mM diethylenetriamine pentaacetic acid (DTPA) (1.42 mL in PBS, pH adjusted to 7.4) and 10 mM of an aqueous tris(2-carboxyethyl)phosphine (TCEP) solution (55.0 μL, 2.75 eq.). After 3 hours at 37° C., the reduced antibody was diluted with PBS (2.21 mL), and pH adjusted with a 100 mM NaOAc solution at pH 5.0 (1.16 mL). To the antibody solution was added 980 μL of DMSO and an excess of MC-GGFG-AM-Compound 139 (179.9 μL; 9 eq.) from a 10 mM DMSO stock solution. The conjugation reaction proceeded at room temperature with mixing for 120 min. An excess of a 100 mM N-acetyl-L-cysteine solution (23.95 μL, 12 eq.) was added to quench the conjugation reaction. Purification and characterization were performed as described in Example 18.

Of note, the DAR8 version of the ADC described above could be generated following the protocol used to generate v29456-MC-GGFG-AM-Compound 139 DAR8 described in Example 17.

v38591-MC-GGFG-AM-Compound 139 DAR4: A solution (0.69 mL) of the v38591 variant (15 mg) in PBS, pH 7.4 was diluted in PBS, pH 7.4 (0.48 mL) and reduced by addition of 5 mM diethylenetriamine pentaacetic acid (DTPA) (0.3 mL in PBS, pH adjusted to 7.4) and 10 mM of an aqueous tris(2-carboxyethyl)phosphine (TCEP) solution (23.2 μL, 2.25 eq.). After 2 hours at 37° C., the reduced antibody was diluted with PBS (0.5 mL), and pH adjusted with a 100 mM NaOAc solution at pH 5.0 (0.25 mL). To the antibody solution was added 167 μL of DMSO and an excess of MC-GGFG-AM-Compound 139 (82.7 μL; 8 eq.) from a 10 mM DMSO stock solution. The conjugation reaction proceeded at room temperature with mixing for 120 min. Purification was performed using Amicon® Ultra centrifugal filter (30 kDa MWCO) pre-equilibrated with 10 mM NaOAc pH 4.5. Characterization was performed as described in Example 18.

v38591-MC-GGFG-AM-Compound 139 DAR8: A solution (2.31 mL) of the v38591 variant (50 mg) in PBS, pH 7.4 was diluted in PBS, pH 7.4 (1.27 mL) and reduced by addition of 5 mM diethylenetriamine pentaacetic acid (DTPA) (1.0 mL in PBS, pH adjusted to 7.4) and 25 mM of an aqueous tris(2-carboxyethyl)phosphine (TCEP) solution (165.2 μL, 12.0 eq.). After 2 hours at 37° C., the reduced antibody was purified using Amicon® Ultra centrifugal filter (30 kDa MWCO) pre-equilibrated with 10 mM NaOAc pH 5.5. The reduced antibody was then diluted with 10 mM NaOAc solution at pH 5.5 (0.75 mL). To the antibody solution was added 126 μL of DMSO and an excess of MC-GGFG-AM-Compound 139 (124.1 μL; 12 eq.) from a 10 mM DMSO stock solution. The conjugation reaction proceeded at room temperature with mixing for 120 min. Purification was performed using Amicon® Ultra centrifugal filter (30 kDa MWCO) pre-equilibrated with 10 mM NaOAc pH 4.5. Characterization was performed as described in Example 18.

### Example 44: Functional Characterization of Anti-NaPi2B Antibody-Drug Conjugate of v40502 (LALADS)—Cell Binding by Flow Cytometry

The ability of the humanized antibody variants v38591 and v40502 and the antibody-drug conjugates v38591-MC-GGFG-AM-Compound 139 DAR 4 and v40502-MC-GGFG-AM-Compound 139 DAR 4 to bind to NaPi2b on the surface of cells was assessed on the endogenous NaPi2b-expressing cancer cell lines IGROV-1 (ovarian carcinoma), HCC-78 (lung adenocarcinoma), H441 (lung adenocarcinoma), and the NaPi2b-negative cancer cell line EBC-1 (lung carcinoma) by flow cytometry. IGROV-1, HCC-78, and H441 cells express endogenous NaPi2b at high to moderate levels, as described in Example 15.

Cells were maintained under standard culture conditions (37° C./5% CO₂) until assay set-up. IGROV-1, HCC-78, H441 and EBC-1 cells were cultured in RPMI 1640, ATCC modification (Thermo Fisher Scientific, Waltham, MA) supplemented with 10% fetal bovine serum (Thermo Fisher Scientific, Waltham, MA). Cells were removed from culture vessels using Cell Dissociation Buffer (Invitrogen, Waltham, MA), seeded at 50,000 cells/well in conical-bottom 96-well plates, and treated with test antibody or antibody-drug conjugate for 24 hours at 4° C. to prevent internalization. Palivizumab (anti-RSV antibody, v21995) was included as a negative control. Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal area geometric mean, proportional to anti-Human AF647 binding) was calculated for the live cell population using FlowJo™ Version 10.8.1 (BD Biosciences, Franklin Lake, NJ) and plotted for each test antibody or antibody-drug conjugate using GraphPad Prism Software Version 9 (GraphPad Software, La Jolla, CA).

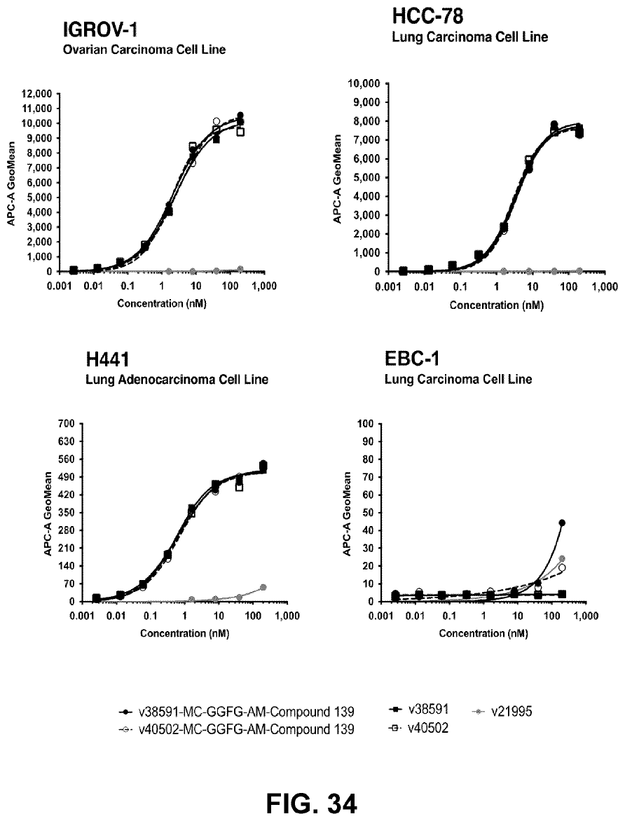

Results are shown in Table 44.1 and plotted in FIG. 34. v38591-MC-GGFG-AM-Compound 139 DAR 4 demonstrated comparable cellular binding to parental antibody v38591 on IGROV-1, HCC-78 and H441 cell lines, indicating that conjugation of naked antibody to MC-GGFG-AM-Compound 139 had minimal impact on the cellular binding capability of the antibody. Both v40502 and v40502-MC-GGFG-AM-Compound 139 DAR 4 demonstrated comparable cellular binding to each other and to v38591 on IGROV-1, HCC-78 and H441 cell lines, indicating that the LALADS modification in the Fc region had no impact on the cellular binding capability of the antibody. Negative control palivizumab (v21995) showed no cellular binding, as expected. In the NaPi2b-negative cancer line EBC-1, all test articles showed minimal binding at high concentration, 200 nM (data not shown).

TABLE 44.1
Binding of v38591 and v40502 ADCs, Parental Antibodies, and Control to Cells

| | | IGROV-1 | | | HCC-78 | | | H441 | | |
|---|---|---|---|---|---|---|---|---|---|---|
| | | | | Curve | | | Curve | | | Curve |
| | | | | Hill | | | Hill | | | Hill |
| | | Bmax | Kd | Slope | | Kd | Slope | | Kd | Slope |
| Sample | DAR | (MFI) | (nM) | (h) | Bmax (MFI) | (Nm) | (h) | Bmax (MFI) | (nM) | (h) |
| v38591-MC-GGFG- | 4 | 10,513 | 0.82 | 2.14 | 7,807 | 1.07 | 3.43 | 521 | 0.74 | 0.64 |
| AM-Compound 139 | | | | | | | | | | |
| v40502-MC-GGFG- | 4 | 10,745 | 0.82 | 2.81 | 7,774 | 1.12 | 3.50 | 518 | 0.81 | 0.72 |
| AM-Compound 139 | | | | | | | | | | |
| v38591 | — | 10,214 | 0.80 | 2.39 | 7,987 | 1.03 | 3.29 | 511 | 0.85 | 0.58 |
| v40502 | — | 9,843 | 1.00 | 1.85 | 7,630 | 1.15 | 2.86 | 512 | 0.78 | 0.64 |
| v21995 | — | NB | NB | NB | NB | NB | NB | NB | NE | NB |

* NB = no binding (apparent Kd value greater than the highest antibody testing concentration >200 nM)

### Example 45: Functional Characterization of v40502 (LALADS) Anti-NaPi2B Antibody and ADC—Internalization

The NaPi2b-mediated internalization of the anti-NaPi2b antibodies v38591 and v40502 and the antibody-drug conjugates v38591-MC-GGFG-AM-Compound 139 DAR 4 and v40502-MC-GGFG-AM-Compound 139 DAR 4 was evaluated in a human cancer cell lines expressing endogenous NaPi2b at high to moderate amounts, as described in Example 15. The cell lines used included IGROV-1 (ovarian carcinoma), HCC-78 (lung adenocarcinoma), and H441 (lung adenocarcinoma) cells and internalization was assessed by flow cytometry following 4 and 24 hours treatment at 2.5 nM of antibody or ADC. The anti-RSV antibody palivizumab (v21995) was used as a negative control.

Briefly, antibodies were fluorescently labeled by coupling to a Fab fragment AF488 conjugate targeting anti-Human IgG Fc (Jackson Immuno Research Labs, West Grove, PA; Cat. No. 109-547-008) at a 1:1 molar ratio in PBS pH 7.4 (Thermo Fisher Scientific, Waltham, MA; Cat. No. 10010-023), for 24 hours at 4° C. Cells were seeded at 50,000 cells/well in RPMI 1640, ATCC modification (Thermo Fisher Scientific, Waltham, MA) supplemented with 10% fetal bovine serum (Thermo Fisher Scientific, Waltham, MA) in 48-well plates and incubated overnight under standard culturing conditions (37° C./5% CO₂) to allow attachment. Coupled antibodies were added to cells the following day at 2.5 nM and incubated under standard culturing conditions for 4-24 hours to allow for internalization. An untreated control (cells incubated with complete growth medium only, no test article) was included for each cell line at each treatment time point. Following incubation, cells were dissociated, washed, and surface AF488 fluorescence was quenched using an anti-AF488 antibody (Life Technologies, Carlsbad, CA; Cat. No. A-11094) at 100 nM for 30 minutes at 4° C. Quenched AF488 fluorescence (internalized fluorescence) was detected by flow cytometry on a BD LSRFortessa™ Cell Analyzer (BD Biosciences, Franklin Lake, NJ) with 1,000 minimum events collected per well. The AF488/FITC-A GeoMean (fluorescence signal area geometric mean, proportional to anti-Human Fab AF488 labelling) was calculated for the live single cell population using FlowJo™ Version 10.8.1 (BD Biosciences, Franklin Lake, NJ). Internalized Fluorescence Fold-Over Untreated values were calculated by dividing FITC-A GeoMean values of treatment wells by the FITC-A GeoMean values of the untreated wells. Internalized Fluorescence Fold-Over Untreated values were plotted using GraphPad Prism Software Version 9 (GraphPad Software, La Jolla, CA).

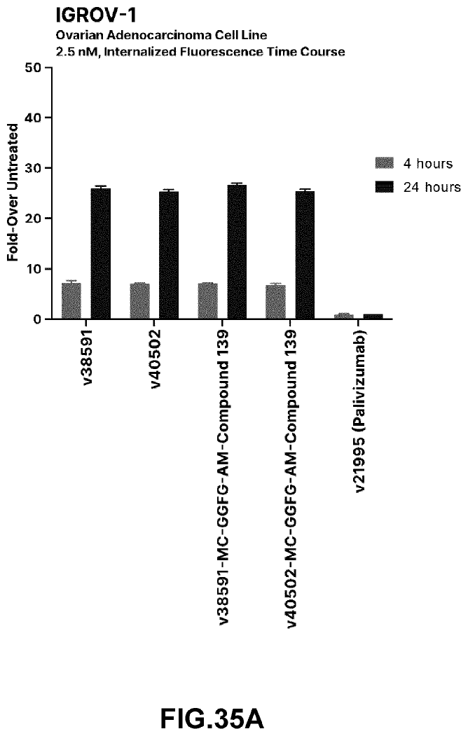

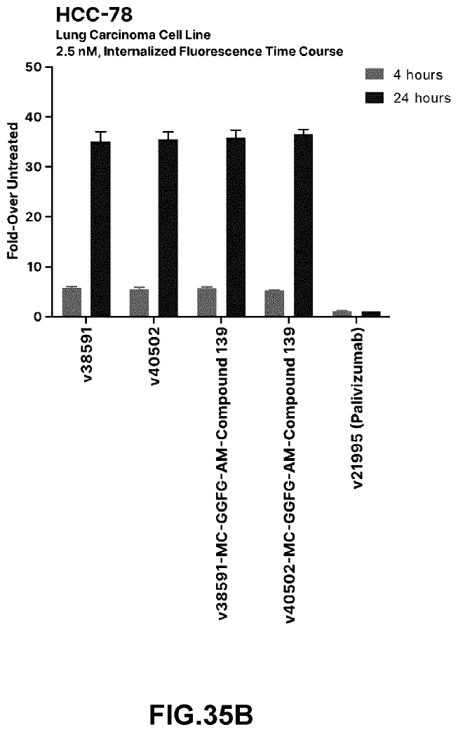

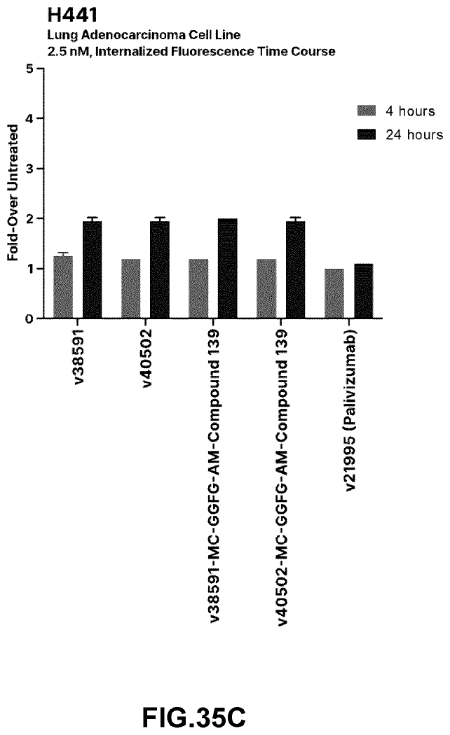

Results are shown in Table 45.1 and plotted in FIGS. 35A-35C. Across all three NaPi2b-expressing cell lines, the antibody-drug conjugates showed levels of internalization comparable to the respective humanized naked antibody. The antibody v40502 showed comparable levels of internalization compared to the antibody v38591. The antibody-drug conjugate v40502-MC-GGFG-AM-Compound 139 showed comparable levels of internalization compared to the antibody-drug conjugate v38591-MC-GGFG-AM-Compound 139. Negative control antibody v21995 demonstrated a negligible degree of internalization comparable to the untreated control (cells incubated with complete growth medium only, no test article).

TABLE 45:1
Internalization of v38591 and v40502 Antibodies and ADCs

| | | Treatment | | | |
|---|---|---|---|---|---|
| | | Time | Internalized Fluorescence Fold-Over Untreated | | |
| Sample (2.5 nM) | DAR | (hours) | IGROV-1 | HCC-78 | H441 |
| v38591 | — | 4 | 7.2 | 5.7 | 1.2 |
| | | 24 | 26.0 | 35.0 | 1.9 |
| v40502 | — | 4 | 7.0 | 5.5 | 1.2 |
| | | 24 | 25.3 | 35.5 | 1.9 |
| v38591-MC-GGFG- | 4.0 | 4 | 7.1 | 5.6 | 1.2 |
| AM-Compound 139 | | 24 | 26.6 | 35.9 | 2.0 |
| v40502-MC-GGFG- | 3.3 | 4 | 6.8 | 5.3 | 1.2 |
| AM-Compound 139 | | 24 | 25.4 | 36.5 | 1.9 |
| v21995 | — | 4 | 0.9 | 1.0 | 1.0 |
| | | 24 | 1.0 | 1.1 | 1.1 |

### Example 46: Cytotoxicity Assessment of v40502 (LALADS) ADC—3D Spheroids

The cell growth inhibition (cytotoxicity) capabilities of the humanized variant v38591 and v40502 conjugated to the drug linker MC-GGFG-AM-Compound 139 at DAR 4 were assessed against NaPi2b-expressing cell line spheroids according to the methods described below. Cell lines used were H1781 (lung adenocarcinoma), and H441 (lung adenocarcinoma). Lifastuzumab vedotin (v18993-MCvc-PABC-MMAE) and ADCs with non-targeted antibody palivizumab (anti-RSV) (v21995 or v22277) were used as controls.

Briefly, cells were seeded in Ultra-Low Attachment 384-well plates at 1,000 cells/well, centrifuged, and incubated for 3 days under standard culturing conditions to allow for spheroid formation and growth. Spheroids were then treated with a titration of test article, generated in cell growth medium, and incubated for 7 days under standard culturing conditions. After incubation, CellTiter-Glo® 3D reagent (Promega Corporation, Madison, WI) was spiked in all wells. Plates were incubated in the dark at room temperature for 1 hour and luminescence was quantified using a BioTek Cytation 5 Cell Imaging Multi-Mode Reader (Agilent Technologies, Inc., Santa Clara, CA). Percent cytotoxicity values were calculated based on untreated wells (cells with complete growth medium only, no test article added), and plotted against test article concentration using GraphPad Prism 9 software (GraphPad Software, La Jolla, CA). EC50 values were calculated based on a non-linear regression log(agonist) versus response, variable slope (four parameters) by GraphPad Prism 9.

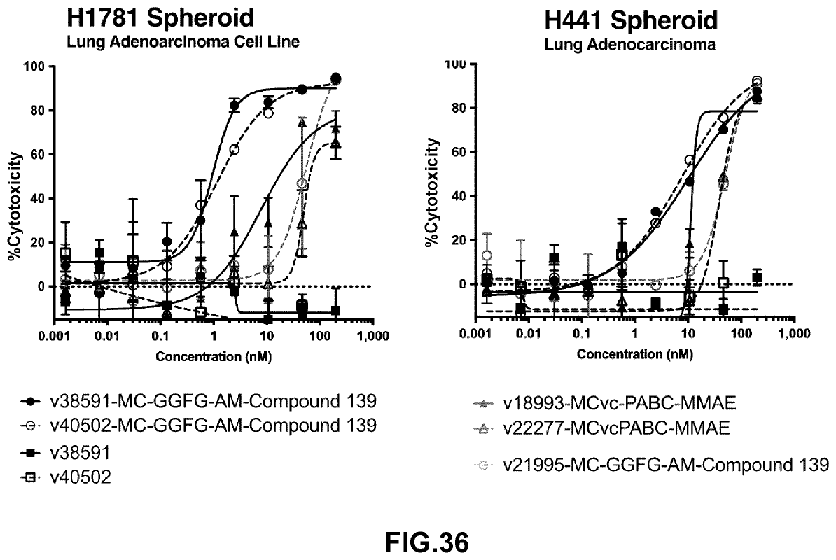

The results are shown in Table 46.1 and representative curves are plotted in FIG. 36. Both v38591-MC-GGFG-AM-Compound 139 and v40502-MC-GGFG-AM-Compound 139 ADCs showed targeted killing against NaPi2b-expressing spheroids, with sub-nanomolar to single-digit nanomolar EC50, while negative control palivizumab ADC showed double-digit nanomolar potency. Both v38591 and v40502 ADCs showed greater potency than control ADC, Lifastuzumab vedotin.

TABLE 46.1
In vitro Cytotoxicity-3D Spheroids-Comparison of
v38591 and v40502 ADCs

| | | EC50 (nM) | |
|---|---|---|---|
| | | H1781 | H441 |
| Sample | DAR | Spheroids | Spheroids |
| v38591-MC-GGFG-AM- | 4.0 | 0.95 | 9.85 |
| Compound 139 | | | |
| v40502-MC-GGFG-AM- | 4.0 | 1.15 | 7.57 |
| Compound 139 | | | |
| v38591 | — | >200 | >200 |
| v40502 | — | >200 | >200 |
| v18993-MCvc-PABC- | 4.1 | 7.94 | 11.36 |
| MMAE | | | |
| V22277-MCvc-PABC- | 4.0 | 49.38 | 38.49 |
| MMAE | | | |
| v21995-MC-GGFG- | 8.0 | 54.69 | 49.65 |
| Compound 139 | | | |

* IC = Incomplete curve (EC50 value greater than the highest antibody testing concentration >200 nM)

### Example 47: ADCC and ADCP Activity OF v40502 (LALADS)

The ability of v38591-MC-GGFG-AM-Compound 139, v38591 antibody, v40502-MC-GGFG-AM-Compound 139, v40502 antibody, v18993 (lifastuzumab) antibody and v21995-MC-GGFG-AM-Compound 139 to mediate antibody-dependent cellular cytotoxicity (ADCC) and antibody-dependent cellular phagocytosis (ADCP) of NaPi2b-expressing IGROV-1 tumor cells was assessed using flow cytometry with human peripheral blood mononuclear cells (PBMCs) and PBMC-derived macrophages (macs) as effector cells, respectively.

In ADCC, Fcγ receptor (FcγR) on the surface of immune effector cells binds to the Fc region of an IgG1 antibody already bound to a target cancer cell. Activation of the FcγR triggers the killer effector cell population within the PBMCs to release perforin, which creates a pore within the target cancer cell plasma membrane, as well as granzyme B, which passes through the created pores and induces target cancer cell death. Similarly, in ADCP, upon activation of FcγR via the Fc region, macs engulf target cancer cells and trigger their death via phagocytosis.

For ADCC assay, 1 human PBMC donor was rested overnight in RPMI+10% ultra-low FBS+100 U/mL recombinant human IL-2. Tumor cells were stained with Cell Tracker™ Green following the manufacturer's protocol (Invitrogen) and mixed with human PBMC effector cells at an effector:target ratio of 25:1.

The cell mixture was added to a serial dilution of v38591-MC-GGFG-AM-Compound 139, v38591 antibody, v40502-MC-GGFG-AM-Compound 139, v40502 antibody, v18993 (lifastuzumab) antibody and v21995-MC-GGFG-AM-Compound 139 or to RPMI+10% ultra-low FBS as untreated control at 37° C. and 5% CO₂ for 4 h. Viability staining was performed using a fixable LIVE/DEAD™ violet dye following the manufacturer's protocol (Invitrogen). Cells were washed once with PBS, centrifuged at 400×g for 5 min and resuspended in FACS buffer (PBS+2% FBS). Cancer cell cytotoxicity was assessed using the BD LSRFortessa™ Cell Analyzer (X-20) HTS (BD Biosciences) flow cytometer by gating for single, dead cancer cells that expressed green fluorescence compared to the total number of cancer cells that expressed green fluorescence. The percent ADCC (% Cytotoxicity) was determined by subtracting the percent cytotoxicity of the untreated cocultures from the percent cytotoxicity of the antibody-treated cocultures.

For ADCP assay, human monocytes were differentiated into macrophages, from human PBMC isolated from 1 healthy donor, by culturing in RPMI+ultra-low 10% FBS+10 ng/mL recombinant human M-CSF for 8 days; culture medium was supplemented every 2-3 days. After 8 days of cultivation, macrophage cellular phenotype (CD14, CD16, and CD11b expression) and morphology were confirmed by flow cytometry and microscopy, respectively. Differentiated macrophages were stained with Cytolight Rapid Red and cancer cells were stained with Cell Tracker Green. Cytolight Rapid Red-stained human-macrophages (red) and Cell Tracker™ Green-labeled cancer cells (green) were mixed at an effector (macrophage cell) to target (cancer cell) ratio of 2:1. The cell mixture was added to a serial dilution of v38591-MC-GGFG-AM-Compound 139, v38591 antibody, v40502-MC-GGFG-AM-Compound 139, v40502 antibody and v21995-MC-GGFG-AM-Compound 139 or to RPMI+10% ultra-low FBS as untreated control and incubated at 37° C. and 5% CO2 for 2 h. A live/dead stain was performed using the violet fixable viability dye following the manufacturer's protocol. Cells were washed once with PBS with 2% FBS, centrifuged and resuspended in FACS buffer. Phagocytic activity was assessed using the BD LSRFortessa™ Cell Analyzer (X-20) HTS (BD Biosciences) flow cytometer by gating for live, single, cells that were double positive for green and red fluorescence. ADCP (%) was determined by subtracting the percent double positive cells of the untreated cocultures from the percent double positive cells of the antibody-treated cocultures. Data analysis for ADCC and ADCP assay was performed with FlowJo™ 10 software.

The results are shown in Table 47.1. v38591-MC-GGFG-AM-Compound 139, v38591 antibody, and v18993 (lifastuzumab) antibody induced dose-dependent ADCC in NaPi2b-expressing IGROV-1 cells with comparable activity after 4 hours of treatment at 12, 48 and 0.0192 nM, with a maximum of 17.21%, 19.96% and 14.41% of ADCC respectively. In contrast, v40502-MC-GGFG-AM-Compound 139, v40502 antibody and v21995-MC-GGFG-AM-Compound 139 DAR 8 did not significantly induce ADCC, with a maximum of 5.32%, 6.56% and 7.01% respectively. Similarly, v38591-MC-GGFG-AM-Compound 139 and v38591 antibody induced ADCP in NaPi2b-expressing IGROV-1 cells with comparable activity after 2 hours of treatment at 100 and 1 nM, with a maximum of 12.3% and 9.7%, respectively. v40502-MC-GGFG-AM-Compound 139, v40502 antibody and v21995-MC-GGFG-AM-Compound 139 DAR 8 did not induce ADCP, with a maximum of 0.2% and 0.4%, respectively.

TABLE 47.1
ADCC and ADCP activity results

| | ADCC | | ADCP | |
|---|---|---|---|---|
| Effector: | (25 PBMCs:1 | | (2 Macs:1 | |
| Target | IGROV-1) | | IGROV-1) | |
| Ratio | Treatment | % | Treatment | % |
| Test Article | conc. (nM) | ADCC | conc. (nM) | ADCP |
| v38591-MC- | 12 | 17.21 | 100 | 12.3 |
| GGFG-AM- | 0.48 | 13.76 | 1 | 9.7 |
| Compound 139 | 0.0192 | 1.46 | — | — |
| DAR 4 | | | | |
| v40502-MC- | 12 | 5.36 | 100 | 0.2 |
| GGFG-AM- | 0.48 | −1.74 | 1 | −0.6 |
| Compound 139 | 0.0192 | −5.29 | — | — |
| DAR 4 | | | | |
| v38591 | 12 | 19.96 | 100 | 9.4 |
| | 0.48 | 16.96 | 1 | 9.7 |
| | 0.0192 | 4.96 | | |
| v40502 | 12 | 6.56 | 100 | −0.9 |
| | 0.48 | −0.24 | 1 | 0.4 |
| | 0.0192 | −2.04 | — | — |
| v18993 | 12 | 14.41 | — | — |
| (lifastuzumab) | 0.48 | 14.01 | — | — |
| | 0.0192 | 5.96 | — | — |
| v21995-MC- | 12 | −3.64 | 100 | −0.1 |
| GGFG-AM- | 0.48 | 7.01 | 1 | −0.2 |
| Compound 139 | 0.0192 | 1.11 | — | — |
| DAR 8 | | | | |

### Example 48: In Vivo Testing of v40502 Antibody-Drug Conjugates of v40502 (LALADS)

### In Vivo Efficacy

In vivo anti-tumor activity of ADCs of humanized anti-NaPi2b antibody variant v38591 and v40502 (LALADS) is assessed in patient-derived xenograft (PDX) and cell-line-derived xenograft (CDX) models of cancer, including the NCI-H441 and/or NCI-H2228 xenograft models of lung cancer. These assessments are carried out according to methods similar to those described in Example 24 for the CDX models and in Examples 35, 37 and 38 for the PDX models.

For the H441 model of lung cancer, 5×10⁶ cells in 0.1 ml 1:1 PBS:Matrigel is implanted subcutaneously into male NU-Foxn1nu mice. When mean tumor volume reaches ~150 mm³ the animals are assigned to groups, n=6 per group, and administered a single IV dose of test articles on study day 0. Tumor volume and body weight were measured twice weekly with a study duration of 28-35 days.

### Pharmacokinetics

In vivo pharmacokinetics of humanized anti-NaPi2b antibody variant v38591 and v40502 (LALADS) and ADCs of humanized anti-NaPi2b antibody variant v38591 and v40502 (LALADS) are assessed in Tg32 humanized FcRn mice.

Pharmacokinetics (PK) of humanized variants v38591 and v40502 (LALADS) and ADCs of humanized variant v38591 and v40502 (LALADS) comprising v38591 and v40502 conjugated to Compound 139 at a DAR4 and DAR8 is assessed in humanized FcRn Tg32 mice, according to methods similar to those described in Example 26.

All test articles are administered at 5 mg/kg to hFcRn Tg32 mice (The Jackson Laboratory, Sacramento, CA; Stock #014565) by intravenous injection. For each test article, blood is collected from n=4 animals by retro-orbital bleed at 1, 3, and 6 hours and 1, 3, 7, 10, 14, 21 days post-dose. Blood is processed to serum and stored frozen at −80° C. in 96-well storage plates prior to pharmacokinetics analysis.

Antibodies in mouse serum samples are captured onto a 384 well plate coated with Goat anti-Human IgG Fc antibody (Jackson 109-005-098). The total antibody is detected with a Goat anti-Human IgG Fab Biotin antibody (Jackson 109-065-097), followed by streptavidin SULFO-TAG (Mesoscale). After adding MSD GOLD Read Buffer A, electrochemiluminescence (ECL) signal by the SULFO-TAG label is measured using the plate reader (Mesoscale).

### Example 49: FcγR and FcRn Binding of V40502 (LALADS) Anti-NaPi2B Antibody by SPR

Surface plasmon resonance (SPR) analysis was carried out to characterize binding of the v40502 (LALADS) anti-NaPi2b antibody to human Fc gamma receptors (FcγR) and human neonatal fragment crystallizable receptor (FcRn) reagents using a Cytiva Biacore™ T200 instrument. The binding characteristics of this antibody were compared to the following reference antibodies and controls: v40554 (LALA) anti-NaPi2b antibody, v38591 (wild-type Fc) anti-NaPi 2b antibody, upifitamab (v40885, wild-type Fc), lifastuzumab (v18993, wild-type Fc), and trastuzumab (wild-type Fe, Roche Diagnostics).

All reference and control antibodies were prepared in-house, except for trastuzumab, which was purchased from the supplier. Upifitamab (v40885) has an identical sequence to MX35 reference antibody (v18992), described elsewhere herein, with an additional amino acid residue between the VL and CL domains of the light chain. Variant 40885 and v18992 do not exhibit any significant functional differences. The anti-NaPi2b antibody v40554 (LALA) differs from v40502 (LALADS) in the Fc region where the v40554 (LALA) lacks the D265S (DS) substitution present in v40502. LALA (L234A/L235A) substitutions in IgG1 antibodies have been shown to reduce binding of the antibody to FcγRs (Wilkinson et al. PLoS One. 2021; 16(12)); however, this reduction is not as complete as that observed with antibodies comprising the LALADS substitutions.

Assessment of binding to FcγRs was carried out as described in Example 40. In the current study, additional anti-NaPi2b reference antibodies were assessed (upifitamab and lifastuzumab), as well as v40554 (LALA) anti-NaPi2b antibody.

Results for binding of antibodies to human FcγR as measured by SPR are shown in Table 49.1. Six FcγR reagents were tested, covering type I, II and III human FcγRs. Rmax values and binding percentage indicated that v40502 (LALADS) exhibited fully abrogated binding to all human FcγRs tested, with nearly no binding responses to these receptors. Variant 40554 (LALA) showed partial binding to CD32aH, CD32aR, CD32bY, CD16aV, and low to no binding to CD64 and CD16aF. In contrast, wild-type Fe anti-NaPi2b antibodies v38591, upifitamab, and lifastuzumab, as well as wild-type Fe anti-HER2 trastuzumab, showed nearly full binding to all the tested FcγRs.

TABLE 49.1
Binding of test antibodies to human FcyR by SPR

| | | | Rmax | K_{D} | Binding |
|---|---|---|---|---|---|
| Ligand | FcγR Sample Variant | FcγR type | (RU) | (M) | percentage |
| Trastuzumab | Sino Biological Inc. | FcγR I CD64 | 232.6 | 4.16E−11 | 90.2% |
| v38591 Wild-type | | | 172.7 | 5.85E−11 | 91.2% |
| v40502 LALADS | | | 0.4 | 1.72E−04 | 0.2% |
| v40554 LALA | | | 14.5 | 3.79E−09 | 6.4% |
| Upifitamab | | | 155.3 | 1.09E−10 | 97.7% |
| Lifastuzumab | | | 224.8 | 9.66E−11 | 76.9% |
| Trastuzumab | v5772 | FcγR IIIA F159 | 120.7 | 1.88E−06 | 90.9% |
| v38591 Wild-type | | CD16aF | 90.2 | 1.13E−06 | 92.5% |
| v40502 LALADS | | | NB | NB | NB |
| v40554 LALA | | | NB | NB | NB |
| Upifitamab | | | 82.4 | 2.38E−06 | 98.7% |
| Lifastuzumab | | | 109.9 | 2.02E−06 | 71.5% |
| Trastuzumab | v5773 | FcγR IIIA V159 | 134.1 | 7.08E−07 | 101.9% |
| v38591 Wild-type | | CD16aV | 92.9 | 4.23E−07 | 96.1% |
| v40502 LALADS | | | 1.5 | 4.47E−07 | 1.8% |
| v40554 LALA | | | 48.4 | 4.02E−06 | 41.5% |
| Upifitamab | | | 90.8 | 9.01E−07 | 109.5% |
| Lifastuzumab | | | 131.5 | 7.38E−07 | 86.4% |
| Trastuzumab | v5774 | FcγR IIA H131 | 124.3 | 8.29E−07 | 82.7% |
| v38591 Wild-type | | CD32aH | 88.4 | 7.99E−07 | 80.0% |
| v40502 LALADS | | | 1.2 | 2.49E-06 | 1.3% |
| v40554 LALA | | | 85.1 | 2.92E−05 | 63.8% |
| Upifitamab | | | 87.1 | 1.01E−06 | 92.1% |
| Lifastuzumab | | | 121.1 | 7.46E−07 | 69.6% |
| Trastuzumab | v5775 | FcγR IIA R131 | 133.9 | 1.21E−06 | 99.9% |
| v38591 Wild-type | | CD32aR | 96.8 | 1.06E−06 | 98.2% |
| v40502 LALADS | | | 0.4 | 2.77E−06 | 0.5% |
| v40554 LALA | | | 68.8 | 2.93E−05 | 58.0% |
| Upifitamab | | | 91.4 | 1.38E−06 | 108.7% |
| Lifastuzumab | | | 126.5 | 9.78E−07 | 81.7% |
| Trastuzumab | v5777 | FcγR IIB Y160 | 133.7 | 5.81E−06 | 101.2% |
| v38591 Wild-type | | CD32bY | 100.3 | 4.98E−06 | 103.2% |
| v40502 LALADS | | | 1.0 | 2.87E−08 | 1.2% |
| v40554 LALA | | | 62.2 | 4.87E−05 | 53.2% |
| Upifitamab | | | 99.7 | 5.78E−06 | 120.6% |
| Lifastuzumab | | | 135 | 3.74E−06 | 88.6% |

Rmax-the maximum observed binding signal;
K_{D}-Equilibrium dissociation constant;
RU-Response units;
M-Molar,
NB-No binding.

This data demonstrates LALADS mutations fully ablate the binding ability of NaPi2b antibody to FcγRs, compared to LALA mutations which reduce binding to FcγRs while maintaining some residual binding.

### Human FcRn Binding Characterization

Binding kinetics was conducted on a Series S CM5 Sensor Chip (Cytiva©, BR100399) which was amine-coupled with Neutravidin (Thermofisher Scientific©, 31000). The chip surface decorated with carboxyl groups was activated with 400 mM EDC (Thermofisher Scientific©, 22980) and 100 nM NHIS (Thermofisher Scientific©, 24500). Neutravidin with concentration of g/mL was injected into flow cell 1 to 4 targeting at 2000 RU with a flow rate of 10 L/min for immobilization. The reaction was inactivated with 1 M Ethanolamine-HCl pH8.5 (Cytiva©, BR100050), following with a buffer wash step using the running buffer, PBS-T (PBS+0.05% (v/v) Tween 20, pH5.9). Biotinylated human FcRn at a concentration of 5 μg/mL was injected and captured by neutravidin as binding ligand at a flow rate of 20 μL/min for 20 sec in flow cell 2, 3, and 4. For affinity analysis, test articles were serially diluted 3-fold to 14.8-1200 nM in PBS-T pH5.9 buffer. The test articles with different dilutions were flowed as analyte using single-cycle kinetics strategy. The flow rate of analyte was 50 μL/min with contact time of 45 sec and dissociation time of 180 sec. Surface regeneration was accomplished using PBS-T (PBS+0.05% (v/v) Tween 20, pH7.4) for 120 sec with a flow rate of 30 μL/min after each cycle. Binding constants were determined with the Biacore™ T200 Evaluation software (version 3.0; GE Healthcare). Blank-subtracted sensorgrams were analyzed using steady state affinity fitting model. Binding percentage was calculated from observed Rmax and theoretical Rmax (Rmax/Rmax_ₜₕₑₒ), based on human FcRn capture density.

Table 49.2 provides the results for analysis of test antibody binding to human FcRn. Rmax values and binding percentage indicated that v40502 (LALADS), v40554 (LALA), v38591 and all reference antibodies showed similar binding level to human FcRn.

TABLE 49.2
Binding of test antibodies to human FcRn by SPR

| | | | Rmax | K_{D} | Binding |
|---|---|---|---|---|---|
| Sample | Surface | Ligand | (RU) | (M) | percentage |
| Trastuzumab | Neutravidin | Biotinlated | 171.5 | 1.03E−07 | 26.89% |
| v38591Wild- | | human FcRn | 182.3 | 1.03E−07 | 28.59% |
| type | | | | | |
| v40502 | | | 168.9 | 8.43E−08 | 26.49% |
| (LALADS) | | | | | |
| v40554 | | | 171.9 | 1.32E−07 | 26.96% |
| (LALA) | | | | | |
| Upifitamab | | | 129.3 | 6.18E−08 | 20.28% |
| Lifastuzumab | | | 159.8 | 6.82E−08 | 25.06% |

Rmax-the maximum observed binding signal;
KD-Equilibrium dissociation constant;
RU-Response units;
M-Molar,

These data indicate that v40502 (LALADS) antibody showed reduction or abrogation of binding to the FcγRs tested, and that the LALADS mutations in the Fc did not affect binding of v40502 (LALADS) to FcRn.

### Example 50: Ability of Anti-NaPi2B Antibody-Drug Conjugates of V40554 (LALA) and v40502 (LALADS) to Mediate ADCC and ADCP

The ability of the anti-NaPi2b antibodies v38591-MC-GGFG-AM-Compound 139 DAR 4 (Fc wild-type), v40554-MC-GGFG-AM-Compound 139 DAR 4 (LALA), v40554 antibody (LALA), v40502-MC-GGFG-AM-Compound 139 DAR 4 (LALADS), v40502 antibody (LALADS), v18992 (Fc wild-type MX-35) antibody, and the palivizumab based ADCs v21995-MC-GGFG-AM-Compound 139 DAR 4, and v40552-MC-GGFG-AM-Compound 139 DAR 4 (LALADS) to mediate antibody-dependent cellular cytotoxicity (ADCC) and antibody-dependent cellular phagocytosis (ADCP) of NaPi2b-expressing IGROV-1 tumor cells was assessed using flow cytometry with human peripheral blood mononuclear cells (PBMCs) and PBMC-derived macrophages (macs) as effector cells, respectively.

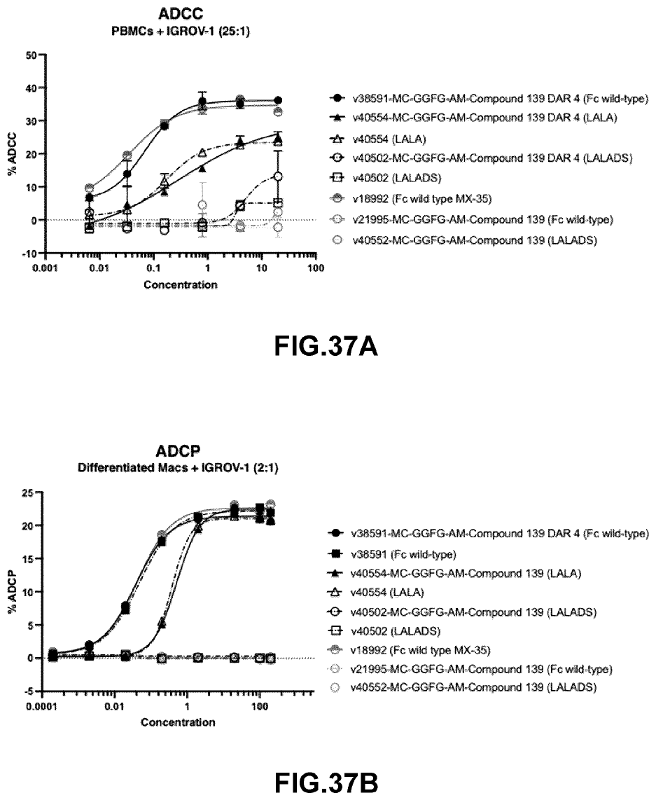

ADCC and ADCP assays were performed as described in Example 47. The results are shown in Table 50.1 and representative curves are plotted in FIG. 37A and FIG. 37B.

TABLE 50.1
ADCC and ADCP activity results

| | ADCC* | ADCP** |
|---|---|---|
| Effector:Target | (25 PBMCs:1 | (2 Macs:1 |
| Ratio | IGROV-1) | IGROV-1) |
| Test Article | EC50 (nM) | EC50 (nM) |
| v38591-MC-GGFG- | 0.069 | 0.037 |
| AM-Compound 139 | | |
| DAR 4 (Fc wild- | | |
| type) | | |
| v38591 (Fc wild- | Not tested | 0.048 |
| type) | | |
| v40554-MC-GGFG- | 0.28 | 0.51 |
| AM-Compound 139 | | |
| DAR 4 (LALA) | | |
| V40554 (LALA) | 0.18 | 0.38 |
| v40502-MC-GGFG- | 4.97 | >200 |
| AM-Compound 139 | | |
| DAR 4 (LALADS) | | |
| V40502 (LALADS) | 3.22 | >200 |
| v18992 (Fc wild- | 0.035 | 0.043 |
| type MX-35) | | |
| v21995-MC-GGFG- | >20 | >200 |
| AM-Compound 139 | | |
| DAR 4 (Fc wild- | | |
| type) | | |
| v40552-MC-GGFG- | >20 | >200 |
| AM-Compound 139 | | |
| DAR 4 (LALADS) | | |

*ADCC = 4-hour incubation
**ADCP = 2-hour incubation

Wild-type Fc ADC v38591-MC-GGFG-AM-Compound 139 DAR 4 and wild-type Fe v18992 (MX-35) induced robust dose-dependent ADCC and ADCP in NaPi2b-expressing IGROV-1 cells with comparable activity. v40554-MC-GGFG-AM-Compound 139 DAR 4 (LALA) and parental antibody v40554 (LALA) resulted in reduced ADCC and ADCP activity compared to wild-type Fc v38591-MC-GGFG-AM-Compound 139 DAR 4. In contrast, v40502-MC-GGFG-AM-Compound 139 DAR 4 (LALADS) and parental antibody v40502 (LALADS) did not show significant ADCP activity at any concentration tested, and showed minimal ADCP activity, only at the highest concentration tested (20 nM), demonstrating the LALADS modification further reduces ADCP and ADCC effector functions of the antibody and ADC tested.

Negative controls v21995-MC-GGFG-AM-Compound 139 DAR 4 and v40552-MC-GGFG-AM-Compound 139 DAR 4 (LALADS) did not show ADCC or ADCP activity, as expected.

These data indicate the v40502-MC-GGFG-AM-Compound 139 DAR 4 does not mediate ADCC- or ADCP-mediated cell death in vitro.

### Example 51: In Vivo Activity of Antibody Drug Conjugates with Wild-Type or Silenced Fc

In vivo anti-tumor activities of Fc WT humanized variant v38591 ADCs and Fc silenced humanized variant v40502 ADCs were assessed in the H441 and LU6802 xenograft models of lung cancer, which both express high levels of NaPi2b. Test articles included v38951 (wild-type Fc) and v40502 (LALADS), v38591-MC-GGFG-AM-Compound 139 DAR 4, v40502-MC-GGFG-AM-Compound 139 DAR 4, and non-targeting controls v21995 (palivizumab with wild-type Fc)-MC-GGFG-AM-Compound 139 DAR 4, and v40552 (palivizumab with LALADS mutations in Fc)-MC-GGFG-AM-Compound 139 DAR 4. The studies were carried out as described below.

For the NCI-H441 model of lung cancer, 5×10⁶ cells in 0.1 ml 1:1 PBS:Matrigel were implanted subcutaneously into male NU-Foxn1nu mice. When mean tumor volume reached ~150 mm³ the animals were assigned to groups, n=6 per group, and administered a single IV dose of test articles on study day 0. Tumor volume and body weight were measured twice weekly with a study duration of 28 days. Treatment groups for NCI-H441 studies are described in Table 51.1.

TABLE 51.1
Treatment groups for H441 Study

| Group | Treatment | DAR | Dose (mg/kg) |
|---|---|---|---|
| 1 | Vehicle | | 0 |
| 2 | v38591 (wild-type Fc)-MC-GGFG-AM-Compound 139 | 4 | 0.8 |
| 3 | v38591 (wild-type Fc)-MC-GGFG-AM-Compound 139 | 4 | 2 |
| 4 | v38591 (wild-type Fc)-MC-GGFG-AM-Compound 139 | 4 | 4 |
| 5 | v40502 (LALADS)-MC-GGFG-AM-Compound 139 | 4 | 0.8 |
| 6 | v40502 (LALADS)-MC-GGFG-AM-Compound 139 | 4 | 2 |
| 7 | v40502 (LALADS)-MC-GGFG-AM-Compound 139 | 4 | 4 |
| 8 | v21995 (wild-type Fc)-MC-GGFG-AM-Compound 139 | 4 | 2 |
| 9 | v40552- (LALADS)MC-GGFG-AM-Compound 139 | 4 | 2 |
| 10 | v38591 (wild-type Fc) | / | 2 |
| 11 | v40502 (LALADS) | / | 2 |

For the LU6802 high NaPi2b expressing model of lung cancer, tumor fragments (~2-3 mm³ diameter) were implanted subcutaneously into female BALB/c nude mice. When mean tumor volume reached ~156 mm³ the animals were assigned to groups, n=6 per group, and administered a single IV dose of test articles on study day 0. Tumor volume and body weight were measured twice weekly with a study duration of 28 days. Treatment groups are described in Table 51.2.

TABLE 51.2
Treatment groups for LU6802 Study

| Group | Treatment | DAR | Dose (mg/kg) |
|---|---|---|---|
| 1 | Vehicle | | 0 |
| 2 | v38591 (wild-type Fc)-MC-GGFG-AM-Compound 139 | 4 | 6 |
| 3 | v38591 (wild-type Fc)-MC-GGFG-AM-Compound 139 | 4 | 12 |
| 4 | v40502 (LALADS)-MC-GGFG-AM-Compound 139 | 4 | 6 |
| 5 | v40502 (LALADS)-MC-GGFG-AM-Compound 139 | 4 | 12 |
| 6 | v21995 (wild-type Fc)-MC-GGFG-AM-Compound 139 | 4 | 12 |
| 7 | v38591 (wild-type Fc) | / | 12 |
| 8 | V40502 (LALADS) | / | 12 |

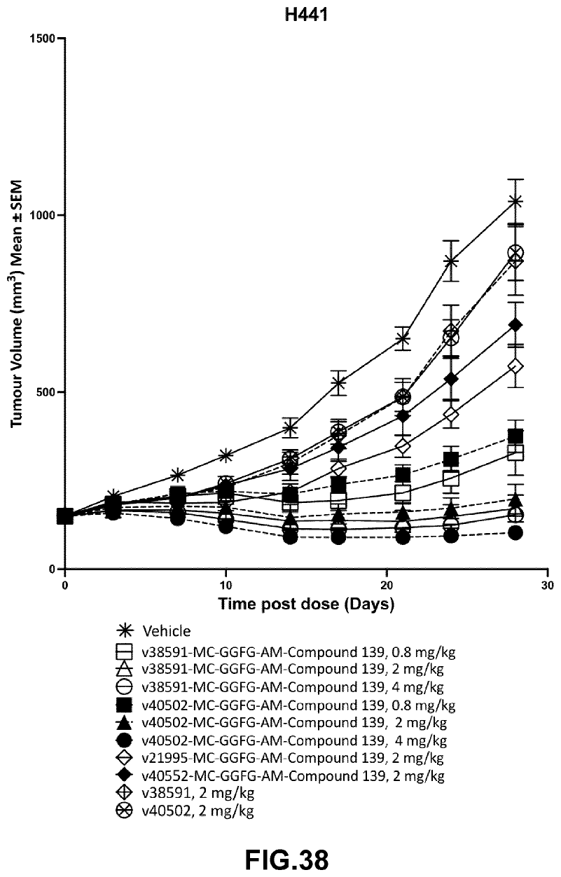

For the H441 model, the results are shown in FIG. 38, demonstrating that v38591-MC-GGFG-AM-Compound 139 with a wild-type Fc and Fc silenced v40502 (LALADS)-MC-GGFG-AM-Compound 139 ADCs inhibited tumor growth to a comparable degree at 0.8, 2 and 4 mg/kg. When dosed at 2 mg/kg, the non-targeting v21995-MC-GGFG-AM-Compound 139 and Fc silenced v40552 (LALADS)-MC-GGFG-AM-Compound 139 ADCs only moderately inhibited tumor growth. The unconjugated mAbs v38591 and v40502 (LALADS) did not result in a strong inhibition of tumor growth.

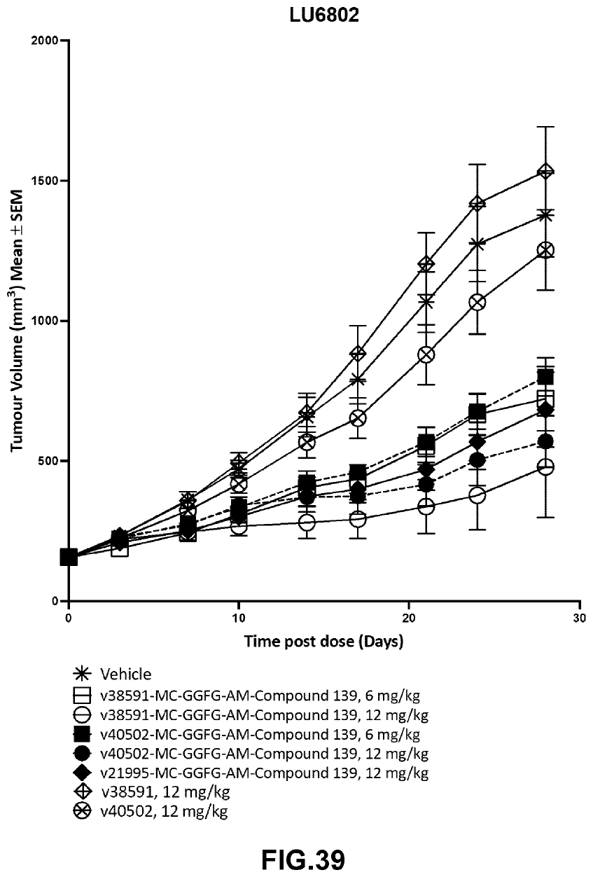

For the LU6802 model, the data is provided in FIG. 39, demonstrating that Fc WT v38591-MC-GGFG-AM-Compound 139 and Fc silenced v40502 (LALADS)-MC-GGFG-AM-Compound 139 ADCs inhibited tumor growth to a comparable degree at 6 and 12 mg/kg. When dosed at 2 mg/kg, the non-targeting v21995-MC-GGFG-AM-Compound 139 also inhibited tumor growth. The unconjugated mAbs v38591 and v40502 (LALADS) did not result in a strong inhibition of tumor growth.

For the lung cancer models H441 and LU6802, a research level immunohistochemistry (IHC) assessment on same study tissue was performed according to the method described in Example 35 and demonstrated each model to express a high level of NaPi2b.

These data demonstrate that v40502 (LALADS) ADCs, despite having a silenced Fe, were able to inhibit tumor growth in the H441 and LU6802 xenograft models to the same extent as the v38591 ADCs having a wild-type Fc.

### Example 52: Identification of Binding Region on Human NaPi2B for Anti-NaPi2B Antibodies

The region of human NaPi2b to which the anti-NaPi2b humanized antibody variant v38591 binds was identified by examining binding of this antibody to chimeric and deletion constructs of human NaPi2b. NaPi2b constructs containing deletions in the main extracellular loop of human NaPi2b, as well as chimeric NaPi2b/NaPi2c constructs concerning this main loop, were generated. These constructs were separately transfected into HEK293-6e cells, which do not endogenously express NaPi2b, and binding of the anti-NaPi2b antibody v38591 to these transfected cells was evaluated by flow cytometry. Reference anti-NaPi2b antibodies MX35 (v18992) and lifastuzumab (v18993) were included as positive controls; reference anti-NaPi2a (polyclonal rabbit anti-human SLC34A1; Atlas Biotechnologies Inc, Edmonton, AB; Cat. No. HPA051255) antibody, known to cross-react with NaPi2c, was included; palivizumab (anti-RSV antibody, v21995) was included as a negative control.

As reference anti-NaPi2b antibodies MX35 and lifastuzumab, as well as v38591, do not bind NaPi2a and NaPi2c (see Example 32), the following constructs were designed for assessing the binding region of v38591: Chimeric constructs in which several regions (as indicated in Table 52.1, numbering as in UniProt entry for NaPi2b, 095436) of the main extracellular loop (deemed to be significantly different in NaPi2b compared to NaPi2c/NaPi2a) in NaPi2b were separately substituted with analogous region of NaPi2c, and NaPi2a in one instance, respectively. These substitutions resulted in five different constructs (Constructs 1-5 in Table 52.1) and were designed such as to comprehensively cover the main loop regions deemed to be significantly different between NaPi2b and NaPi2c/NaPi2a. As a complementary strategy, two additional constructs were designed in which the relevant region of the main loop was deleted such as to mimic NaPi2c/NaPi2a main extracellular loop (Constructs 6-7 in Table 52.1). Clones were generated at GenScript Biotech (Piscataway, NJ).

Constructs were expressed in HEK293-6e cells as follows. Cells were maintained under standard culture conditions (37° C./5% CO₂) with shaking at 110 rpm for suspension and cultured in FreeStyle™ 293 Expression Medium (Thermo Fisher Scientific, Waltham, MA) supplemented with 1% fetal bovine serum (Thermo Fisher Scientific, Waltham, MA) and 1× Penicillin-Streptomycin (Thermo Fisher Scientific, Waltham, MA). Cells were transfected with clones expressing the constructs listed in Table 52.1, at 1 μg of DNA per 1 million cells, using 293Fectin™ Transfection Reagent (Thermo Fisher Scientific, Waltham, MA) and Opti-MEM™ I Reduced Serum Medium (Thermo Fisher Scientific, Waltham, MA), and incubated for 24 hours under standard culture conditions (37° C./5% CO₂/110 rpm). Following transfection, cells were seeded at 50,000 cells/well in conical-bottom 96-well plates and treated with a serial dilution of test antibody for 24 hours at 4° C. to prevent internalization. Cells were treated with final concentrations of all humanized antibodies of 200, 20, or 2 nM. Cells were treated with final concentrations of the reference anti-NaPi2a rabbit antibody of 50, 5, or 0.5 nM. Following incubation, cells were washed and stained with 2 μg/mL anti-Human IgG Fc AF647 conjugate (Jackson ImmunoResearch Labs, West Grove, PA; Cat. No. 109-605-098) or anti-Rabbit IgG F(ab′)2 AF647 conjugate (Jackson ImmunoResearch Labs, West Grove, PA; Cat. No. 111-605- 047). Following incubation and washing, fluorescence was detected by flow cytometry on a BD LSRFortessa™ Cell Analyzer (BD Biosciences, Franklin Lake, NJ) with 1,000 minimum events collected per well. The AF647/APC-A GeoMean (fluorescence signal geometric mean, proportional to anti-Human AF647 binding) was calculated for the single cell population using FlowJo™ Version 10.8.1 (BD Biosciences, Franklin Lake, NJ). Background-subtracted AF647 Mean Fluorescence Intensity (MFI) values were calculated by subtracting the average MFI of three technical replicates of unstained (no primary antibody) cells.

Results are tabulated in Table 52.2. The antibody variant v38591 demonstrated dose-dependent binding to Construct 1, Construct 2, Construct 3, and Construct 4, to similar levels compared to reference antibodies MX35 (v18992) and lifastuzumab (v18993), indicating that the amino acid regions 239-259 (Construct 1), 280-301 (Construct 2), 304-321 (Construct 3), and 341-358 (Construct 4) do not affect binding of these antibodies to NaPi2b. None of the anti-NaPi2b antibodies demonstrated binding to Construct 5 and Construct 6, indicating that amino acid regions 304-348 (Construct 5) and 317-333 (Construct 6) contain the binding region of v38591, MX35, and lifastuzumab on NaPi2b. The antibody variant v38591 demonstrated dose-dependent binding to Construct 7, while MX35 (v18992) demonstrated impaired binding to Construct 7 and lifastuzumab (v18993) demonstrated no binding to Construct 7. This indicates that the amino acid region 317-326 (Construct 7) is not necessary for binding of v38591 to NaPi2b but is involved in binding of reference antibodies MX35 (v18992) and lifastuzumab (v18993) to NaPi2b. Taken together, these results indicate that the epitope for v38591 is within amino acids 327-340 of the NaPi2b extracellular main loop.

The antibody v38591 and reference anti-NaPi2b antibodies MX35 (v18992) and lifastuzumab (v18993) exhibited high levels of binding to cells transfected with full length human NaPi2b, as expected. Reference anti-NaPi2a antibody demonstrated dose-dependent binding to cells transfected with Construct 1, Construct 2, Construct 3, Construct 4, and Construct 5, which all contain a portion of the NaPi2a or NaPi2c extracellular loop, but not Construct 6, Construct 7, or full length human NaPi2b. Negative control palivizumab (v21995) showed no binding to any transfected cell lines.

TABLE 52.1
NaPi2b plasmid chimeric and deletion constructs

| Plasmid | | |
|---|---|---|
| DNA | CL_# | Details |
| Construct 1 | CL_#29914 | Swap in NaPi2c in 239-259 |
| Construct 2 | CL_#29915 | Swap in NaPi2c in 280-301 |
| Construct 3 | CL_#29916 | Swap in NaPi2c in 304-321 |
| Construct 4 | CL_#13435 | Swap in NaPi2c in 341-358 |
| Construct 5 | CL_#13436 | Swap in NaPi2a in 304-348 |
| Construct 6 | CL_#29919 | NaPi2b del 317-333 |
| Construct 7 | CL_#29921 | NaPi2b del 317-326 |
| huNaPi2b | CL_#13432 | Full length human NaPi2b |

TABLE 52.2
Mean fluorescence intensity values demonstrating binding of anti-NaPi2b antibodies to
mutant NaPi2b constructs

| | | Background-Subtracted AF647 Mean Fluorescence Intensityª | | | | |
|---|---|---|---|---|---|---|
| | | | | | | Anti- |
| | | | | | | SLC34A1 |
| DNA | Concentration | | MX35 | lifastuzumab | palivizumab | Rb |
| Plasmid | (nM) | v38591 | (v18992) | (v18993) | (v21995) | antibody^{b} |
| Construct 1 | 200.0 | 2,158 | 1,718 | 1,508 | 12 | 616 |
| | 20.0 | 1,856 | 1,816 | 1,697 | -27 | 859 |
| | 2.0 | 1,554 | 1,893 | 1,232 | -30 | 99 |
| Construct 2 | 200.0 | 1,409 | 1,356 | 1,232 | -30 | 230 |
| | 20.0 | 1,337 | 1,246 | 1,198 | -64 | 222 |
| | 2.0 | 832 | 660 | 473 | -69 | 70 |
| Construct 3 | 200.0 | 810 | 802 | 943 | -5 | 375 |
| | 20.0 | 1,116 | 930 | 920 | 39 | 411 |
| | 2.0 | 743 | 594 | 609 | 19 | 40 |
| Construct 4 | 200.0 | 933 | 764 | 744 | 12 | 511 |
| | 20.0 | 993 | 979 | 1,049 | 22 | 250 |
| | 2.0 | 725 | 611 | 726 | 1 | 146 |
| Construct 5 | 200.0 | 204 | 219 | 302 | 40 | 339 |
| | 20.0 | 103 | 79 | 52 | 123 | 546 |
| | 2.0 | -12 | 16 | 25 | -2 | 119 |
| Construct 6 | 200.0 | 210 | -2 | 183 | 2 | 291 |
| | 20.0 | -7 | 67 | -3 | 1 | 283 |
| | 2.0 | -38 | -59 | -91 | 11 | 666 |
| Construct 7 | 200.0 | 626 | 298 | 21 | -97 | 123 |
| | 20.0 | 981 | 233 | 35 | -85 | 1 |
| | 2.0 | 677 | 76 | 56 | -34 | 369 |
| huNaPi2b | 200.0 | 989 | 979 | 893 | 19 | 107 |
| | 20.0 | 1,422 | 1,249 | 1,372 | 13 | 97 |
| | 2.0 | 948 | 870 | 1,044 | -22 | 596 |

^{a}Background-Subtracted AF647 Mean Fluorescence Intensity (MFI) was calculated by subtracting the average MFI of three technical replicates of unstained (no primary antibody) cells.
^{b}Anti-SLC34A1 rabbit antibody was treated at 50.0, 5.0, and 0.5 nM to cells.

### Example 53: Target-Independent Cellular Binding of v40554 (Lala) and v40502 (LALADS) Anti-NaPi2b ADCS

Target-independent binding of anti-NaPi2b antibody-drug conjugates v38591-MC-GGFG-AM-Compound 139 DAR 4 (Fc wild-type), v40554-MC-GGFG-AM-Compound 139 DAR 4 (LALA), and v40502-MC-GGFG-AM-Compound 139 DAR 4 (LALADS) was evaluated in vitro on normal cell types that do not express NaPi2b, but do express a range of Fc receptors for IgG (FcγRs). This experiment is meant to assess potential FcγR-mediated ADC binding in cells that do not express NaPi2b.

Cell lines studied included human Kupffer cells (liver macrophages), peripheral blood mononuclear cells (PBMC)-derived human macrophages, THP-1 cells (acute monocytic leukemia cell line), HMEC-1 cells (dermal microvascular endothelial cell line), and HCE-2 cells (human corneal epithelial cell line). Binding was assessed by flow cytometry following 4-hour treatment at 500 nM of test article at 4° C. The anti-RSV antibody palivizumab ADC (v21995-MC-GGFG-AM-Compound 139 DAR 4) was used as a negative control.

THP-1 (American Type Culture Collection; TIB-202), HMEC-1 (American Type Culture Collection; CRL-3243), and HCE-2 (American Type Culture Collection; CRL-3582) cells were maintained under supplier-recommended culture conditions until assay set-up; PBMC-derived macrophages were differentiated from human monocytes isolated from 1 healthy donor and cultured in RPMI+10% ultra-low IgG FBS+10 ng/mL recombinant human M-CSF for 7 days; culture medium was supplemented every 2-3 days. After 7 days of cultivation, macrophage cellular phenotype morphology and FcγR expression were confirmed by microscopy and flow cytometry, respectively. Human primary Kupffer cells (Lonza; Cat. No. HLKC-500K) were derived from human liver tissue and cryopreserved by supplier; Kupffer cells were thawed in supplier-provided media kit at time of assay set-up.

### Assessment of Surface Fc Receptor Levels and NaPi2b Expression Levels in Cells

Surface Fc receptors for IgG (FcγRs), CD64 (FcγRI) and CD32A (FcγRIIA), CD32B/C (FcγRIIB/C) and CD16a (FcγRIIIA), were measured on cell lines using quantitative flow cytometry. APC mouse anti-human FcγRI/CD64 antibody (BD Biosciences, Franklin Lake, NJ; Cat. No. 561189), PE mouse anti-human FcγRIIA/CD32A antibody (Biolegend, San Diego, CA; Cat. No. 365503), APC mouse anti-human FcγRIIB/C (Biolegend, San Diego, CA; Cat. No. 398406) and FITC mouse anti-human FcγRIIIA/CD16a antibody (Novus Biologics, Centennial, CO; Cat. No. NB10064346F) were used to fluorescently label all cell types and beads with five distinct levels of human antibody binding capacity (ABC), at a pre-determined excess concentration of conjugated antibody or left untreated for 30 minutes at 4° C. Cells and beads were washed in FACS buffer and analyzed on the BD LSRFortessa™ Cell Analyzer and processed using FlowJo™ Version 10.8.1 (BD Biosciences, Franklin Lake, NJ). The mean fluorescence intensity for all beads populations were plotted against relevant ABC values using Bangs Laboratories QuickCal v 2.3 calibration line template for QSC® anti-Mouse IgG Lot #15515. Surface receptor protein expression for cell lines was calculated based on a monovalent binding model and therefore equivalent to the background subtracted ABC (SABC); the mean fluorescence for the untreated cells from each respective cell lines was used as a background value for determination of SABC. Additionally, NaPi2b mRNA expression in similar cell line categories as those assessed was identified using normalized Transcripts Per Million (nTPM) values extracted from The Human Protein Atlas (Uhlen M et al. Science. 2015 Jan. 23; 347(6220):1260419; Karlsson M et al., Sci Adv. 2021 Jul. 28; 7(31):2169). This information is provided in Table 53.1.

### Assessment of NaPi2b-Independent Binding to Cells

For assessment of NaPi2b-independent binding, antibodies or ADCs were fluorescently labeled by coupling to a Fab fragment AF488 conjugate targeting anti-Human IgG Fc (Jackson Immuno Research Labs, West Grove, PA; Cat. No. 109-547-008) at a 1:1 molar ratio in PBS pH 7.4 (Thermo Fisher Scientific, Waltham, MA; Cat. No. 10010-023), for 24 hours at 4° C. Adherent cells were removed from culture vessels using Cell Dissociation Buffer (Invitrogen, Waltham, MA); cells were seeded 20,000-50,000 cells/well in conical-bottom 96-well plates, treated with coupled antibodies at 500 nM, and incubated at 4° C. for 4 hours to prevent internalization. Following incubation, cells were washed, and fluorescence was detected by flow cytometry on a BD LSRFortessa™ Cell Analyzer (BD Biosciences, Franklin Lake, NJ) with 1,000 minimum events collected per well. The 7-AAD (Thermo Fisher Scientific, Waltham; Cat. No. A1310)/PerCP-Cy5.5 channel was used to obtain the live cell population, and AF488/FITC-A GeoMean (fluorescence signal area geometric mean, proportional to anti-Human Fab AF488 labelling) was calculated for the live single cell population using FlowJo™ Version 10.8.1 (BD Biosciences, Franklin Lake, NJ).

Results are shown in Table 53.1. Assessment of surface Fc receptor levels indicated that human Kupffer cells (liver macrophages), PBMC-derived macrophages, and THP-1 monocytes exhibited high surface expression of FcγRI and FcγRIIA, in contrast with human corneal epithelial cell lines HMEC-1 and HCE-2, which expressed low FcγRI. Additionally, human Kupffer cells (liver macrophages) and PBMC-derived macrophages also expressed high surface FcγRIIB/C and FcγRIIIA.

Cellular binding of Fc wild type v38591-MC-GGFG-AM-Compound 139 was comparable to Fc wild type control ADC v21995-MC-GGFG-AM-Compound 139 (RSV-targeting) on all normal cell types tested. In cells expressing FcγRs (Kupffer liver macrophages, PBMC-derived macrophages, and THP-1 monocytes), v38591-MC-GGFG-AM-Compound 139 demonstrated higher cellular binding than v40554-MC-GGFG-AM-Compound 139 (LALA) and v40502-MC-GGFG-AM-Compound 139 (LALADS). In cells expressing low levels of FcγRs (HMEC-1 and HCE-2 corneal epithelial cells), ADCs showed comparable levels of binding.

TABLE 53.1
Single-point NaPi2b-independent binding of v38591, v40554, and v40502 ADCs,
and Control to Cells

| | Human | PBMC- | | | | |
|---|---|---|---|---|---|---|
| | Kupffer | derived | | | | |
| Cell Lines | Cells | macrophages | THP-1 | HMEC-1 | HCE-2 | |
| CD64 (FcγRI) Expression (CD64/cell) | 19,677 | 262,744 | 67,864 | 4 | 35 | |
| CD32A (FcγRIIA) Expression (CD32A/ | 29,806 | 375,164 | 137,530 | 32 | Not | |
| cell) | | | | | assayed | |
| CD32B/C (FcγRIIB/C) Expression | 105,367 | 47,584 | 1,816 | 34 | Not | |
| (CD32B/C/cell) | | | | | assayed | |
| CD16a (FcγRIIIA) Expression(CD16a/cell) | 71,792 | 1,290,534 | 3,967 | 4,046 | Not | |
| | | | | | assayed | |
| Sample (500 nM) | DAR | Surface Binding Fluorescence (MFI) | | | | |
| v38591-MC-GGFG-AM-Compound | 4.0 | 2,341 | 15,234 | 811 | 251 | 763 |
| 139 | | | | | | |
| Fold-difference from | | 1.0 | 1.0 | 1.0 | 1.0 | 1.0 |
| Fc wild-type v38591 ADC | | | | | | |
| v40554-MC-GGFG-AM-Compound | 4.2 | 1,279 | 5,839 | 229 | 215 | 752 |
| 139 | | | | | | |
| Fold-difference from | | 1.8 | 2.6 | 3.5 | 1.2 | 1.0 |
| Fc wild-type v38591 ADC | | | | | | |
| v40502-MC-GGFG-AM-Compound | 3.8 | 1,292 | 4,158 | 222 | 216 | 799 |
| 139 | | | | | | |
| Fold-difference from | | 1.8 | 3.7 | 3.7 | 1.2 | 1.0 |
| Fc wild-type v38591 ADC | | | | | | |
| v21995-MC-GGFG-AM-Compound | 4.0 | 1,914 | 11,731 | 768 | 233 | 718 |
| 139 | | | | | | |
| Fold-difference from | | 1.2 | 1.3 | 1.1 | 1.1 | 1.1 |
| Fc wild-type v38591 ADC | | | | | | |

### Example 54—Target-Independent Cytotoxicity of v40554 (Lala) and v40502 (LALADS) Anti-NaPi2b ADCs on Human Erythroid and Myeloid Lineages—Colony-Forming Unit Assay

The non-specific (target-independent) cytotoxicity of v38591-MC-GGFG-AM-Compound 139 DAR 4 (Fe wild-type), v40502-MC-GGFG-AM-Compound 139 DAR 4 (LALADS) and Compound 139 was assessed on human erythroid and myeloid progenitors differentiated from human bone marrow-isolated hematopoietic stem cells (HSC) CD34-positive (CD34+), using the colony-forming unit (CFU) assay, performed by Discovery Life Sciences (DLS). Reference ADC trastuzumab-MC-GGFG-AM-DXd1 DAR 8 (HER2 targeting ADC, Fc wild-type) was used in the assay as a positive control. The non-specific (target-independent) cytotoxicity of ADCs in human erythroid and myeloid progenitors is of interest as, in the context of off-target toxicities, uptake and trafficking of intact ADCs into normal cells can occur by different mechanisms following administration of ADC in vivo, including target-dependent, for example, NaPi2b-dependent internalization, or target-independent mechanisms, for example, FcγR-dependent internalization or non-specific endocytosis (Mahalingaiah et al., Pharmacol Ther. 2019 August; 200:110-125). Profiling the in vitro target-independent cytotoxicity of ADCs to erythroid and myeloid progenitors may further understanding of their interactions in vivo.

Briefly, 20,000 human bone marrow-isolated HSC CD34+ were seeded in a semi-solid methylcellulose-based media formulation containing recombinant human interleukin-3 (rhIL-3, 10 ng/mL), rh granulocyte-macrophage-colony-stimulating factor (rhGM-CSF, 10 ng/mL), rh stem cell factor (rhSCF, 50 ng/mL) and Erythropoietin (Epo, 3 U/mL), to induce differentiation into clonogenic progenitors of human erythroid (CFU-erythroid CFU-E, burst-forming unit-erythroid BFU-E), granulocyte-monocyte (CFU-granulocyte-macrophage CFU-GM) and multipotential (CFU-granulocyte-erythroid-monocyte-megakaryocyte CFU-GEMM). The test articles v38591-MC-GGFG-AM-Compound 139 DAR 4, v40502-MC-GGFG-AM-Compound 139 DAR 4, trastuzumab-MC-GGFG-AM-DXd1 DAR 8 and Compound 139 were prepared at different concentrations to obtain a 6-points curve and added to the assay media with cells. Test article solvents (phosphate buffer saline for v38591-MC-GGFG-AM-Compound 139 DAR 4, v40502-MC-GGFG-AM-Compound 139 DAR 4, trastuzumab-MC-GGFG-AM-DXd1 DAR 8 and dimethyl sulfoxide for Compound 139) were also used for control treatment. Cells were then incubated for 14 days at standard culture conditions. After treatment, erythroid and myeloid colonies were assessed microscopically and scored. The colonies were divided into the following categories based on size and morphology: CFU-E, BFU-E, CFU-GM and CFU-GEMM. Percent survivability values were calculated based on the number of colonies after treatment over the number of colonies after solvent controls treatment and plotted against test article concentration using GraphPad Prism 9 software (GraphPad Software, La Jolla, CA). IC50 values were calculated based on a non-linear regression log(inhibitor) versus response, variable slope (four parameters) by GraphPad Prism 9.

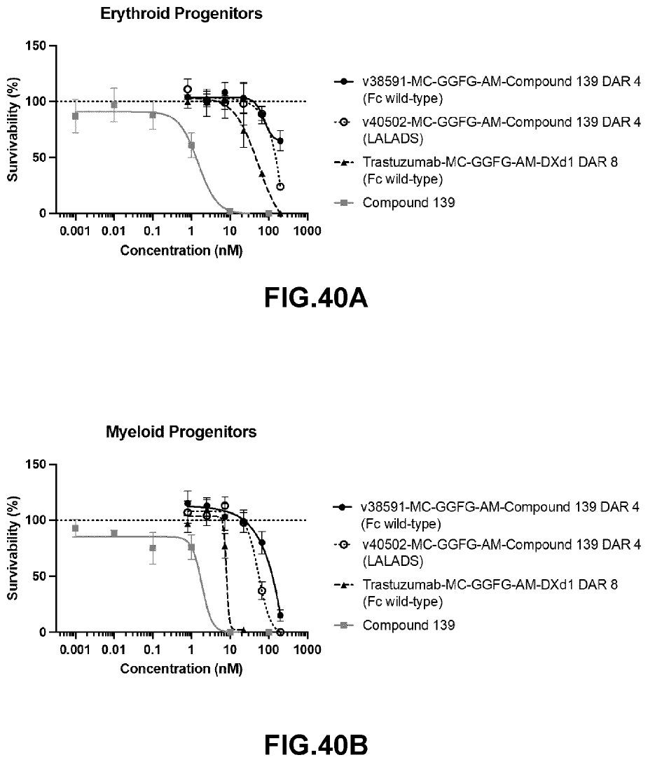

The results are shown in Table 54.1 and % survivability curves are plotted in FIG. 40A and FIG. 40B. In the erythroid progenitors, v38591-MC-GGFG-AM-Compound 139 DAR 4 (Fc wild-type) and v40502-MC-GGFG-AM-Compound 139 DAR 4 (LALADS) did not induce significant cytotoxicity at the concentrations tested. Both Fc wild-type and Fc-silenced ADCs exhibited moderate killing, mainly at the highest concentrations tested (200 nM), as shown in the % survivability curves, yielding IC50 values of >200 nM and 132 nM, respectively. Trastuzumab-MC-GGFG-AM-DXd 1 DAR 8 (Fc wild-type) induced higher cytotoxicity, with an IC50 of 43.2 nM.

In the myeloid progenitors, both Fc wild-type and Fc-silenced ADCs, v38591-MC-GGFG-AM-Compound 139 DAR 4 (Fc wild-type) and v40502-MC-GGFG-AM-Compound 139 DAR 4 (LALADS), exhibited moderate cytotoxicity, with IC50 values of 108 nM and 57.61 nM, respectively. Trastuzumab-MC-GGFG-AM-DXd1 DAR 8 (Fc wild-type) induced higher cytotoxicity, with an IC50 of 18.9 nM.

Compound 139 was more cytotoxic than v38591-MC-GGFG-AM-Compound 139 DAR 4, v40502-MC-GGFG-AM-Compound 139 DAR 4 and trastuzumab-MC-GGFG-AM-DXd1 DAR 8 both in erythroid and myeloid progenitors, with an IC50 of 1.32 and 1.75 nM, respectively.

TABLE 54.1
Erythroid and myeloid progenitors CFU assay results

| | Human IC50 (nM) | |
|---|---|---|
| | Erythroid | Myeloid |
| | progenitors | progenitors |
| v38591-MC-GGFG-AM-Compound 139 | >200 | 108 |
| DAR 4 (wild-type Fc) | | |
| v40502-MC-GGFG-AM-Compound 139 | 132 | 57.6 |
| DAR 4 (LALADS) | | |
| Trastuzumab-MC-GGFG-AM-DXd1 | 43.2 | 18.9 |
| DAR 8 (wild-type Fc) | | |
| Compound 139 | 1.32 | 1.75 |

### Example 55: Cellular Binding and Internalization of V40502 (LALADS) and v40554 (LALA) Anti-NaPi2B ADCs in Normal Lung Cells by Flow Cytometry

The NaPi2b-independent cellular binding and internalization of the anti-NaPi2b antibody-drug conjugates v38591-MC-GGFG-AM-Compound 139 DAR 4, v40554-MC-GGFG-AM-Compound 139 DAR 4 and v40502-MC-GGFG-AM-Compound 139 DAR 4 can be evaluated in normal or tumor lung cells not expressing NaPi2b target, including healthy human alveolar macrophages (Accegen, Cat. No. ABC-H0034X), non-small cell lung cancer (NSCLC) human alveolar macrophages (Accegen, Cat. No. ABC-H0034X) and healthy human interstitial macrophages (Accegen, Cat. No. ABC-H0034X). The cellular binding and internalization of these ADCs can also be evaluated in normal healthy human alveolar type II pneumocytes (Accegen, ABC-TC5515), which have been reported to express NaPi2b (Bergstrom et al. 2016. IASLC; Uhlen M et al. Science. 2015 Jan. 23; 347(6220):1260419; Karlsson M et al., Sci Adv. 2021 Jul. 28; 7(31):2169). The anti-RSV antibody palivizumab ADC (v21995-MC-GGFG-AM-Compound 139) can be used as a negative control.

The normal lung cells are cultured according to vendor instructions upon thawing, using specialized growth medium to maintain adequate cell viability until assay set-up.

Surface Fc receptors for IgG (FcγRs), including CD64 (FcγRI) and CD16a (FcγRIIIA), as well as surface NaPi2b, are measured on normal lung cells using quantitative flow cytometry. Fluorophore-conjugated anti-FcγRI/CD64 mouse antibody, anti-FcγRIIIA/CD16a mouse antibody, and anti-NaPi2b human antibody are used to fluorescently label cells and beads with five distinct levels of mouse or human antibody binding capacity (ABC), at pre-determined excess level of fluorophore-conjugated antibody or are left untreated for 30 minutes at 4° C. Cells and beads are washed in FACS buffer and are analyzed on the BD LSRFortessa™ Cell Analyzer, and are processed using FlowJo™ Version 10.8.1 (BD Biosciences, Franklin Lake, NJ). The mean fluorescence intensity for all beads populations is plotted against relevant ABC values, using Bangs Laboratories QuickCal v 2.3 calibration line template. Surface protein expression for all cells is calculated based on a monovalent binding model and therefore equivalent to the background subtracted ABC (SABC); the mean fluorescence for the untreated cells from each respective cell lines is used as a background value for determination of SABC.

Cellular binding and internalization of ADCs in normal lung cells is assessed by flow cytometry following 24-hour and 4-hour incubation with test article. Test articles can be fluorescently coupled to a Fab fragment AF488 conjugate targeting anti-Human IgG Fc (Jackson Immuno Research Labs, West Grove, PA; Cat. No. 109-547-008) at a 1:1 molar ratio in PBS, pH 7.4.

For cellular binding assessment in normal lung cells, including cells not expressing NaPi2b target, such as healthy and NSCLC alveolar macrophages, and normal lung cells expressing NaPi2b, such as type II pneumocytes, a titration of fluorescently-labeled ADCs is incubated with 10,000-40,000 cells/well in 96-well plates at 4° C., to prevent internalization, for 4 and 24 hours. Following incubation, cells are washed and are analyzed by flow cytometry on a BD LSRFortessa™ Cell Analyzer (BD Biosciences, Franklin Lake, NJ) with 1,000 minimum events collected per well. The 7-AAD (Thermo Fisher Scientific, Waltham; Cat. No. A1310)/PerCP-Cy5.5 channel is used to obtain the live cell population, and AF488/FITC-A GeoMean (fluorescence signal area geometric mean, proportional to anti-Human Fab AF488 labelling) is calculated for the live single cell population using FlowJo™ Version 10.8.1 (BD Biosciences, Franklin Lake, NJ).

For assessment of ADC internalization in normal lung cells not expressing NaPi2b target, adherent cells are seeded at 20,000-50,000 cells/well in complete growth medium in 96-well plates, treated with fluorescently-labeled ADCs and incubated under standard culturing conditions (37° C./5% CO₂) for 4 hours to allow for internalization. Following incubation, cells are washed, and surface AF488 fluorescence is quenched using an anti-AF488 antibody (Life Technologies, Carlsbad, CA; Cat. No. A-11094) at 200 nM for 40 minutes at 4° C. Quenched AF488 fluorescence (internalized fluorescence) is detected by flow cytometry on a BD LSRFortessa™ Cell Analyzer (BD Biosciences, Franklin Lake, NJ) with 1,000 minimum events collected per well. The 7-AAD (Thermo Fisher Scientific, Waltham; Cat. No. A1310)/PerCP-Cy5.5 channel is used to obtain the live cell population, the AF488/FITC-A GeoMean (fluorescence signal area geometric mean, proportional to anti-human Fab AF488 labelling) is calculated for the live single cell population using FlowJo™ Version 10.8.1 (BD Biosciences, Franklin Lake, NJ).

### Example 56: Cellular Binding and Internalization of V40502 (LALADS) and v40554 (LALA) Anti-NaPi2B ADCs in Normal and Cancer Cells by Flow Cytometry

The NaPi2b-independent cellular binding and internalization of the anti-NaPi2b antibody-drug conjugates v38591-MC-GGFG-AM-Compound 139 DAR 4 (Fc-wildtype), v40554-MC-GGFG-AM-Compound 139 DAR 4 (LALA) and v40502-MC-GGFG-AM-Compound 139 DAR 4 (LALADS) can be evaluated in a panel of normal and cancer cells corresponding to various different tissues not expressing NaPi2b target, including human dermal fibroblast HDFa cell line (ThermoFisher Scientific, C0135C), embryonic epithelial small intestine FHs 74 Int cell line (ATCC CCL-241), fibroblast large intestine CCD-33Co cell line (ATCC CRL-1539), embryonic epithelial large intestine FHC cell line (ATCC CRL-1831), human T lymphocyte Jurkat cell line (Riken BRC, RCB0806), acute myeloid leukemia MOLM-13 cell line (DSMZ, ACC 554) and NOMO-1 cell line (DSMZ, ACC 542), bone marrow-derived chronic myelogenous leukaemia MEG-01 cell line (Millipore Sigma, 94012401) and K-562 cell line (ATCC, CCL-243). The anti-RSV antibody palivizumab ADC (v21995-MC-GGFG-AM-Compound 139) can be used as a negative control.

The normal and cancer cells are cultured according to vendor instructions upon thawing, using specialized growth medium to maintain adequate cell viability until assay set-up.

Surface Fc receptors for IgG (FcγRs), including CD64 (FcγRI) and CD16a (FcγRIIIA), as well as surface NaPi2b, are measured on normal lung cells using quantitative flow cytometry. Fluorophore-conjugated anti-FcγRI/CD64 mouse antibody, anti-FcγRIIIA/CD16a mouse antibody, and anti-NaPi2b human antibody are used to fluorescently label cells and beads with five distinct levels of mouse or human antibody binding capacity (ABC), at pre-determined excess level of fluorophore-conjugated antibody or are left untreated for 30 minutes at 4° C. Cells and beads are washed in FACS buffer and are analyzed on the BD LSRFortessa™ Cell Analyzer, and are processed using FlowJo™ Version 10.8.1 (BD Biosciences, Franklin Lake, NJ). The mean fluorescence intensity for all beads populations is plotted against relevant ABC values, using Bangs Laboratories QuickCal v 2.3 calibration line template. Surface protein expression for all cells is calculated based on a monovalent binding model and therefore equivalent to the background subtracted ABC (SABC); the mean fluorescence for the untreated cells from each respective cell lines is used as a background value for determination of SABC.

Cellular binding and internalization of ADCs in normal lung cells is assessed by flow cytometry following 24-hour and 4-hour incubation with test article. Test articles can be fluorescently coupled to a Fab fragment AF488 conjugate targeting anti-Human IgG Fc (Jackson Immuno Research Labs, West Grove, PA; Cat. No. 109-547-008) at a 1:1 molar ratio in PBS, pH 7.4.

For cellular binding assessment in normal lung cells, including cells not expressing NaPi2b target, such as healthy and NSCLC alveolar macrophages, and normal lung cells expressing NaPi2b, such as type II pneumocytes, a titration of fluorescently-labeled ADCs is incubated with 10,000-40,000 cells/well in 96-well plates at 4° C., to prevent internalization, for 4 and 24 hours. Following incubation, cells are washed and are analyzed by flow cytometry on a BD LSRFortessa™ Cell Analyzer (BD Biosciences, Franklin Lake, NJ) with 1,000 minimum events collected per well. The 7-AAD (Thermo Fisher Scientific, Waltham; Cat. No. A1310)/PerCP-Cy5.5 channel is used to obtain the live cell population, and AF488/FITC-A GeoMean (fluorescence signal area geometric mean, proportional to anti-Human Fab AF488 labelling) is calculated for the live single cell population using FlowJo™ Version 10.8.1 (BD Biosciences, Franklin Lake, NJ).

For assessment of ADC internalization in normal lung cells not expressing NaPi2b target, adherent cells are seeded at 20,000-50,000 cells/well in complete growth medium in 96-well plates, treated with fluorescently-labeled ADCs and incubated under standard culturing conditions (37° C./5% CO₂) for 4 hours to allow for internalization. Following incubation, cells are washed, and surface AF488 fluorescence is quenched using an anti-AF488 antibody (Life Technologies, Carlsbad, CA; Cat. No. A-11094) at 200 nM for 40 minutes at 4° C. Quenched AF488 fluorescence (internalized fluorescence) is detected by flow cytometry on a BD LSRFortessa™ Cell Analyzer (BD Biosciences, Franklin Lake, NJ) with 1,000 minimum events collected per well. The 7-AAD (Thermo Fisher Scientific, Waltham; Cat. No. A1310)/PerCP-Cy5.5 channel is used to obtain the live cell population, the AF488/FITC-A GeoMean (fluorescence signal area geometric mean, proportional to anti-human Fab AF488 labelling) is calculated for the live single cell population using FlowJo™ Version 10.8.1 (BD Biosciences, Franklin Lake, NJ).

### Example 57: In Vivo Activity of Antibody Drug Conjugates with Wild-Type or Silenced Fc Region

In vivo anti-tumor activities of Fc WT humanized variant v38591 ADCs and Fc silenced humanized variant v40502 ADCs were assessed in the NaPi2b expressing UT5321 patient derived xenograft (PDX) model of endometrial cancer in NOD/SCID mice. Test articles included v38951 (wild-type Fc) and v40502 (LALADS), v38591-MC-GGFG-AM-Compound 139 DAR 4, v40502-MC-GGFG-AM-Compound 139 DAR 4, and non-targeting controls v21995 (palivizumab with wild-type Fc)-MC-GGFG-AM-Compound 139 DAR 4, and v40552 (palivizumab with LALADS mutations in Fc)-MC-GGFG-AM-Compound 139 DAR 4. The study was carried out as described below.

For the UT5321 moderate NaPi2b expressing model of endometrial cancer, tumor fragments (~2-3 mm diameter) were implanted subcutaneously into female NOD/SCID mice. When mean tumor volume reached ~156 mm³ the animals were assigned to groups, n=6 per group, and administered a single IV dose of test articles on study day 0. Tumor volume and body weight were measured twice weekly with a study duration of 28 days. Treatment groups are described in Table 57.1.

TABLE 57.1
Treatment groups for UT5321 Study

| | | | Dose |
|---|---|---|---|
| Group | Treatment | DAR | (mg/kg) |
| 1 | Vehicle | | 0 |
| 2 | v38591 (wild-type Fc)-MC-GGFG- | 4 | 3 |
| | AM-Compound 139 | | |
| 3 | v38591 (wild-type Fc)-MC-GGFG- | 4 | 6 |
| | AM-Compound 139 | | |
| 5 | v40502 (LALADS)-MC-GGFG-AM- | 4 | 3 |
| | Compound 139 | | |
| 6 | v40502 (LALADS)-MC-GGFG-AM- | 4 | 6 |
| | Compound 139 | | |
| 8 | v21995 (wild-type Fc)-MC-GGFG-AM- | 4 | 6 |
| | Compound 139 | | |
| 9 | v40552 (LALADS)-MC-GGFG-AM- | 4 | 6 |
| | Compound 139 | | |

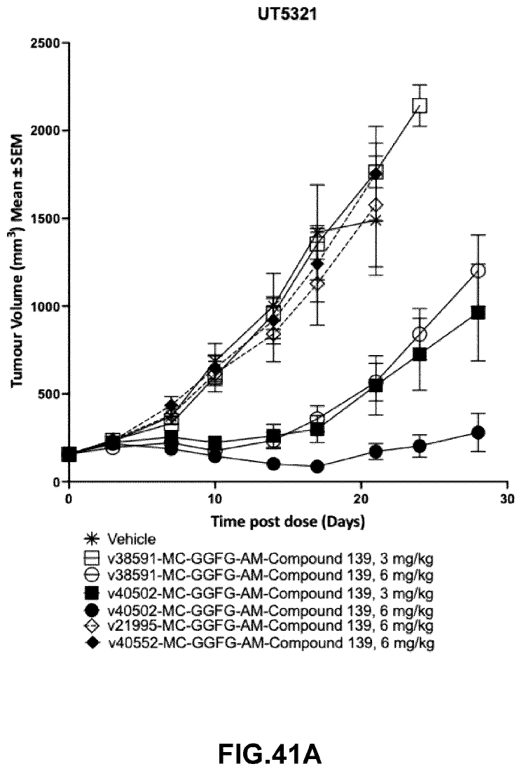

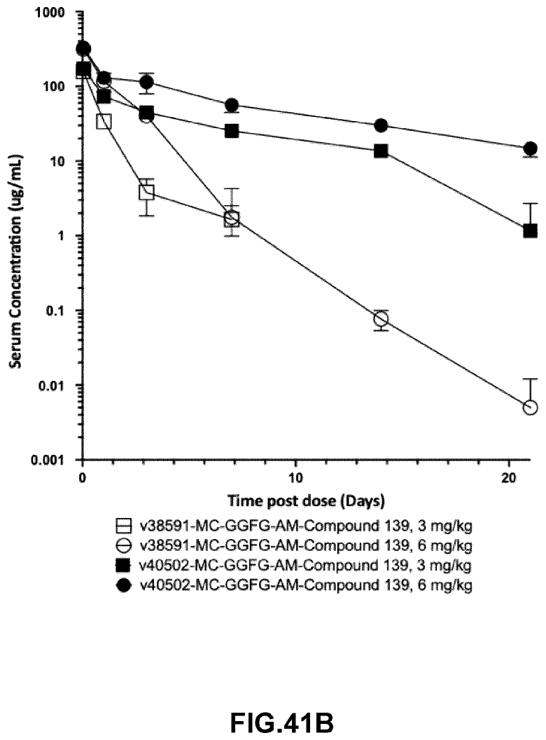

For the UT5321 model, the results are shown in FIG. 41A, demonstrating that when dosed at 3 mg/kg, the v38591-MC-GGFG-AM-Compound 139 ADC with a wild-type Fc was inactive, while the Fc silenced v40502 (LALADS)-MC-GGFG-AM-Compound 139 ADC inhibited tumor growth. When dosed at 6 mg/kg, the Fc silenced v40502 (LALADS)-MC-GGFG-AM-Compound 139 ADC inhibited tumor growth to a greater degree than v38591-MC-GGFG-AM-Compound 139 ADC with a wild-type Fc. When dosed at 6 mg/kg, the non-targeting v21995-MC-GGFG-AM-Compound 139 and Fc silenced v40552 (LALADS)-MC-GGFG-AM-Compound 139 ADCs were inactive.

The total antibody pharmacokinetics of the v38591-MC-GGFG-AM-Compound 139 ADC with a wild-type Fc and the Fc silenced v40502 (LALADS)-MC-GGFG-AM-Compound 139 ADC in the UT5321 endometrial cancer PDX model in NOD/SCID mice was assessed by an MSD assay. The results are shown in FIG. 41B, demonstrating that in the NOD/SCID UT5321 PDX model, v38591-MC-GGFG-AM-Compound 139 ADC with a wild-type Fc has a shorter half-life and greater clearance than the Fc silenced v40502 (LALADS)-MC-GGFG-AM-Compound 139 ADC.

For the endometrial cancer model UT5321, a research level immunohistochemistry (IHC) assessment of the study xenograft tumor tissue was performed according to the method described in Example 35 and demonstrated the model to express a moderate level of NaPi2b.

These data demonstrate that the v40502 (LALADS) ADC with a silenced Fc, inhibited tumor growth to a greater degree than the v38591-ADC with a wild-type Fc in the UT5321 PDX model of endometrial cancer in NOD/SCID mice, and that this was associated with an inferior PK profile of the v38591-ADC with a wild-type Fc compared to the v40502 (LALADS) ADC with a silenced Fc in this model.

### Example 58: Target-Independent Binding of v40554 (Lala) and v40502 (LALADS) Anti-NaPi2b ADCs to Cells Including Lung, Hepatic and Gastric Cells

The target-independent binding of anti-NaPi2b antibody-drug conjugates v38591-MC-GGFG-AM-Compound 139 DAR 4 (Fc wild-type), v40554-MC-GGFG-AM-Compound 139 DAR 4 (LALA), and v40502-MC-GGFG-AM-Compound 139 DAR 4 (LALADS) was evaluated in vitro on cell types that do not express NaPi2b, and do express a range of Fc gamma receptors for IgG (FcγRs), to assess potential FcγR-mediated (target-independent) ADC binding.

Cell lines used included human liver macrophage Kupffer cells (Lonza, Basel, Switzerland; Cat. No. HLKC-500K), peripheral blood mononuclear cells (PBMC)-derived macrophages (STEMCELL Technologies, Vancouver, Canada; Cat. No. 70025), acute monocytic leukemia cell line THP-1 (ATCC, Manassas, VA; Cat. No. TIB-202), liver endothelial cell line SK-HEP-1 (ATCC; Cat. No. HTB-52), healthy alveolar macrophages (Accegen, Fairfield, NJ; Cat. No. ABC-H0034X), large intestine fibroblast cell line CCD-33Co (ATCC; Cat. No. CRL-1539), embryonic epithelial large intestine cell line FHC (ATCC; Cat. No. CRL-1831), chronic myeloid leukemia cell line K562 (ATCC; Cat. No. CCL-243), T lymphocyte cell line Jurkat (Riken BRC, Kyoto, Japan; Cat. No. RCB0806), and acute myeloid leukemia cell line MOLM-13 (DSMZ, Braunschweig, Germany; Cat. No. ACC 554).

Binding of ADCs was assessed by flow cytometry following 4-hour treatment at 500 nM of test article at 4° C. Fe gamma receptor levels were assessed by flow cytometry in the cell lines evaluated to correlate their expression with binding of Fc-modified and Fc-wild type ADCs.

### Cell Culturing

THP-1, SK-HEP-1, CCD-33Co, K562, FHC, Jurkat, and MOLM-13 were maintained under supplier-recommended culture conditions until assay set-up. PBMC-derived macrophages were differentiated from monocytes isolated from 1 healthy human donor and cultured in RPMI 1640 (ATCC Modification) (Thermo Fisher Scientific, Waltham, MA; Cat. No. A1049101)+10% ultra-low IgG FBS (Thermo Fisher Scientific; Cat. No. 12483-020)+10 ng/mL recombinant human M-CSF (R&D Systems, Minneapolis, MN; Cat. No. 216-MC) for 7 days; culture medium was supplemented every 2-3 days. After 7 days of cultivation, macrophage phenotype was confirmed by cellular morphology and FcγR expression using microscopy and flow cytometry, respectively. Primary Kupffer cells were derived from human liver tissue and cryopreserved by the supplier; Kupffer cells were thawed in the supplier-provided media kit (Lonza; Cat. No. MKC-500BK) at time of assay set-up. Primary alveolar macrophages from two healthy human donors were acquired and cryopreserved by the supplier; alveolar macrophages were thawed in the supplier-provided media kit (Accegen; Cat. No. ABM-H0034X) at time of assay set-up. Assessment of surface Fc gamma receptor levels and NaPi2b expression levels in cells

Surface expression of Fc receptors for IgG (FcγRs), CD64 (FcγRI) and CD32A (FcγRIIA), CD32B/C (FcγRIIB/C) and CD16A (FcγRIIIA), was measured on cell lines using quantitative flow cytometry. Cells were first incubated with Human TruStain FcX™ (BioLegend, San Diego, CA; Cat No. 422302), diluted at 1:25 in FACS buffer (PBS, pH 7.4 (Thermo Fisher Scientific; Cat. No. 10010-023)+2% FBS (Thermo Fisher Scientific; Cat. No. 12483-020)), for 10 minutes at 4° C. to block FcγR binding. Cells were then washed in FACS buffer. APC mouse anti-human FcγRI/CD64 antibody (BD Biosciences, Franklin Lake, NJ; Cat. No. 561189), PE mouse anti-human FcγRIIA/CD32A antibody (Biolegend; Cat. No. 365503), APC mouse anti-human FcγRIIB/C (Biolegend; Cat. No. 398406) and FITC mouse anti-human FcγRIIIA/CD16A antibody (Novus Biologics, Centennial, CO; Cat. No. NB10064346F) were used to fluorescently label all cell types and Quantum™ Simply Cellular® (QSC) anti-mouse beads (Bangs Laboratories, Fishers, IN; Cat. No. 815B) containing five distinct levels of mouse antibody binding capacity (ABC), at a pre-determined excess concentration of conjugated antibody, or left untreated, for 30 minutes at 4° C. Cells and beads were washed in FACS buffer and analyzed on the BD LSRFortessa™ Cell Analyzer and processed using FlowJo™ Version 10.8.1 (BD Biosciences, Franklin Lake, NJ). The mean fluorescence intensity for all bead populations were plotted against relevant ABC values using Bangs Laboratories QuickCal v 2.3 calibration line template for QSC® anti-Mouse IgG Lot #15515. Surface receptor protein expression for cell lines was calculated based on a monovalent binding model and therefore equivalent to the background subtracted ABC (SABC); the mean fluorescence for the untreated cells from each respective cell lines was used as a background value for determination of SABC. Additionally, NaPi2b mRNA expression in similar cell line categories as those assessed was identified using normalized Transcripts Per Million (nTPM) values extracted from The Human Protein Atlas (Uhlen M et al. 2015. Science; Karlsson M et al., 2021. Sci Adv). This information is provided in Table 58.1.

TABLE 58.1
FcγR Quantification and NaPi2b Expression of Certain Cell Lines Tested

| | | NaPi2b | | | FcγRIIB/ | |
|---|---|---|---|---|---|---|
| | | Expression | FcγRI | FcγRIIA | C(CD32B/ | FcγRIIIA |
| Cell Line | Cell Type | (nTPM)¹ | (CD64/cell) | (CD32A/cell) | C/cell) | (CD16a/cell) |
| Kupffer cells | Liver | 0.0 | 19,677 | 29,806 | 105,367 | 71,792 |
| (human) | macrophage | | | | | |
| PBMC-derived | Blood | 2.0 | 262,744 | 375,164 | 47,584 | 1,290,534 |
| macrophages | macrophage | | | | | |
| (donor RG1565) | | | | | | |
| THP-1 | Blood monocyte | 0.1 | 67,864 | 137,530 | 1,816 | 3,967 |
| SK-HEP-1 | Liver | 0.0 | 1,156 | 1,188 | 1,194 | 36,762 |
| | endothelial cells | | | | | |
| Healthy alveolar | Alveolar | 3.1-11.8 | 326,830 | 215,273 | 3,365 | 32,963 |
| macrophages | macrophages | | | | | |
| (donor M34) | | | | | | |
| Healthy alveolar | Alveolar | 3.1-11.8 | 166,502 | 321,790 | 22,379 | 31,892 |
| macrophages | macrophages | | | | | |
| (donor M35) | | | | | | |
| CCD-33Co | Colon fibroblast | 0.0 | 1,530 | 17,356 | 713 | Not assayed |
| FHC | Colon epithelial | 0.0 | 720 | 1,828 | 860 | Not assayed |
| K562 | Chronic myeloid | 0.0 | -64 | 131,424 | -6 | Not assayed |
| | leukemia | | | | | |
| Jurkat | T lymphocyte | 0.2 | 34 | 272 | 79 | Not assayed |
| MOLM-13 | Acute myeloid | 0.2 | 2,342 | 53,109 | 843 | Not assayed |
| | leukemia | | | | | |

¹Extracted from similar cell line categories from Human Protein Atlas (Uhlén M et al. 2015. Science; Karlsson M et al., 2021. Sci Adv), in Transcripts Per Million (nTPM).

### Assessment of Target-Independent Binding of ADCs to Cells

For assessment of NaPi2b-independent binding, ADCs were fluorescently labeled by coupling to a Fab fragment AF488 conjugate targeting anti-Human IgG Fe (Jackson Immuno Research Labs, West Grove, PA; Cat. No. 109-547-008) at a 1:1 molar ratio in PBS pH 7.4 (Thermo Fisher Scientific; Cat. No. 10010-023), for 24 hours at 4° C. Adherent cells were removed from culture vessels using Cell Dissociation Buffer (Invitrogen, Waltham, MA; Cat. No. 13151-014); cells were seeded 10,000-50,000 cells/well in conical-bottom 96-well plates, treated with coupled antibodies at 500 nM, and incubated at 4° C. for 4 hours to prevent internalization. Following incubation, cells were washed, stained with dead cell stain 7-Aminoactinomycin D (7-AAD) (Thermo Fisher Scientific; Cat. No. A1310), and fluorescence was detected by flow cytometry on a BD LSRFortessa™ Cell Analyzer (BD Biosciences) with 1,000 minimum events collected per well. The live cell population was determined by dead cell exclusion on the 7-AAD/PerCP-Cy5.5 channel, and AF488/FITC-A GeoMean (fluorescence signal area geometric mean, proportional to anti-Human Fab AF488 labelling) was calculated for the live single cell population using FlowJo™ Version 10.8.1 (BD Biosciences).

Results are shown in Table 58.2. Assessment of surface Fc receptor levels indicated that PBMC-derived macrophages and healthy alveolar macrophages expressed the highest levels of FcγRI, Kupffer cells and THP-1 cells expressed moderate amounts of FcγRI, and SK-HEP-1, CCD-33Co, FHC, K562, Jurkat, and MOLM-13 cell lines expressed low to negligible amounts of FcγRI. PBMC-derived macrophages, healthy alveolar macrophages, THP-1, and K562 expressed the highest levels of FcγRIIA, followed by Kupffer cells, CCD-33Co, and MOLM-13 expressing moderate amounts of FcγRIIA, and SK-HEP-1, FHC, and Jurkat cells expressing low to negligible amounts of FcγRIIA. In contrast, Kupffer cells expressed the highest amounts of FcγRIIB/C, followed by PBMC-derived macrophages and healthy alveolar macrophages from donor M35 expressing moderate amounts of FcγRIIB/C, and THP-1, SK-HEP-1, healthy alveolar macrophage from donor M34, CCD-33Co, FHC, K562, Jurkat, and MOLM-13 cells expressing low to negligible amounts of FcγRIIB/C. Finally, PBMC-derived macrophages exhibited high surface expression of FcγRIIIA, while Kupffer cells, SK-HEP-1, and healthy alveolar macrophages from both donors expressed moderate amounts of FcγRIIIA, and THP-1 expressed a low amount of FcγRIIIA.

TABLE 58.2
Single-point target-independent binding of v38591, v40554, and v40502 ADCs

| | | v38591-MC-GGFG- | | v40554-MC-GGFG- | | | |
|---|---|---|---|---|---|---|---|
| | | AM-Compound 139 | | AM-Compound 139 | | v40502-MC-GGFG-AM- | |
| | | | Fold- | | Fold- | Compound 139 | |
| | | | difference | | difference | | Fold-difference |
| | | | from v38591 | | from v38591 | | from v38591 wt |
| Cell Line | Cell Type | MFI | wt ADC | MFI | wt ADC | MFI | ADC |
| Kupffer cells | Liver Macrophage | 2,274 | 1.0 | 1,279 | 1.78 | 1,239 | 1.84 |
| (human) | | | | | | | |
| PBMC-derived | Blood Macrophage | 15,234 | 1.0 | 5,839 | 2.61 | 4,158 | 3.66 |
| macrophages | | | | | | | |
| THP-1 | Blood Monocyte | 811 | 1.0 | 229 | 3.54 | 222 | 3.65 |
| SK-HEP-1 | Liver Endothelial | 532 | 1.0 | 521 | 1.02 | 525 | 1.01 |
| Healthy alveolar | Alveolar | 4,837 | 1.0 | 2,500 | 1.93 | 2,537 | 1.91 |
| macrophages | Macrophage | | | | | | |
| (donor M34) | | | | | | | |
| Healthy alveolar | Alveolar | 8,022 | 1.0 | 4,595 | 1.75 | 3,738 | 2.15 |
| macrophages | Macrophage | | | | | | |
| (donor M35) | | | | | | | |
| CCD-33Co | Colon Fibroblast | 508 | 1.0 | 553 | 0.92 | 576 | 0.88 |
| FHC | Colon Epithelial | 1,352 | 1.0 | 1,331 | 1.02 | 1,305 | 1.04 |
| K562 | Chronic Myeloid | 716 | 1.0 | 717 | 1.00 | 688 | 1.04 |
| | Leukemia | | | | | | |
| Jurkat | T Lymphocyte | 114 | 1.0 | 111 | 1.03 | 119 | 0.96 |
| MOLM-13 | Acute Myeloid | 267 | 1.0 | 247 | 1.08 | 262 | 1.02 |
| | Leukemia | | | | | | |

In cells expressing high overall levels of Fe gamma receptors (Kupffer cells, PBMC-derived macrophages, THP-1, and healthy alveolar macrophages from both donors M34 and M35), Fc-modified ADCs, v40554-MC-GGFG-AM-Compound 139 (LALA) and v40502-MC-GGFG-AM-Compound 139 (LALADS), demonstrated lesser cellular binding compared to v38591-MC-GGFG-AM-Compound 139 (Fc wild-type). In cells expressing low levels of Fc gamma receptors, particularly FcγRI (SK-HEP-1, CCD-33Co, FHC, K562, Jurkat, and MOLM-13), v40554-MC-GGFG-AM-Compound 139 (LALA) and v40502-MC-GGFG-AM-Compound 139 (LALADS) showed similar and minimal cellular binding comparable to v38591-MC-GGFG-AM-Compound 139 (Fc wild-type)

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

### Additional Sequence Tables (SEQ ID NOs:36-59, 61-63, 69)

TABLE A
Clone Numbers for Variants

| | Clone Numbers | | | |
|---|---|---|---|---|
| Variant | H1 | H2 | L1 | L2 |
| 18993 | CL_#11677 | CL_#11678 | CL_#11695 | CL_#11695 |
| 18992 | CL_#11698 | CL_#11699 | CL_#11700 | CL_#11700 |
| 22277 | CL_#11009 | CL_#11010 | CL_#16635 | CL_#16635 |
| 21995 | CL_#16603 | CL_#16603 | CL_#16635 | CL_#16635 |
| 29449 | CL_#21506 | CL_#21506 | CL_#21509 | CL_#21509 |
| 29450 | CL_#21505 | CL_#21505 | CL_#21509 | CL_#21509 |
| 29451 | CL_#21504 | CL_#21504 | CL_#21509 | CL_#21509 |
| 29452 | CL_#21503 | CL_#21503 | CL_#21509 | CL_#21509 |
| 29453 | CL_#21506 | CL_#21506 | CL_#21508 | CL_#21508 |
| 29454 | CL_#21505 | CL_#21505 | CL_#21508 | CL_#21508 |
| 29455 | CL_#21504 | CL_#21504 | CL_#21508 | CL_#21508 |
| 29456 | CL_#21503 | CL_#21503 | CL_#21508 | CL_#21508 |
| 29457 | CL_#21506 | CL_#21506 | CL_#21507 | CL_#21507 |
| 29458 | CL_#21505 | CL_#21505 | CL_#21507 | CL_#21507 |
| 29459 | CL_#21504 | CL_#21504 | CL_#21507 | CL_#21507 |
| 29460 | CL_#21503 | CL_#21503 | CL_#21507 | CL_#21507 |
| 38591 | CL_#29371 | CL_#29371 | CL_#21508 | CL_#21508 |

TABLE B
Clone amino acid Sequences (Full Chain)

| Clone | | | SEQ ID |
|---|---|---|---|
| Number | Region | Sequence | NO |
| CL_#11009 | Full heavy | GQVTLRESGPALVKPTQTLTLTCTFSGFSLSTSGMSVGWIRQPPGKAL | 36 |
| | chain | EWLADIWWDDKKDYNPSLKSRLTISKDTSKNQVVLKVTNMDPADTA | |
| | | TYYCARSMITNWYFDVWGAGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVYPPSRDELTKNQVSLTCLVKGF | |
| | | YPSDIAVEWESNGQPENNYKTTPPVLDSDGSFALVSKLTVDKSRWQQ | |
| | | GNVFSCSVMHEALHNHYTQKSLSLSPG | |
| CL_#11010 | Full heavy | GQVTLRESGPALVKPTQTLTLTCTFSGFSLSTSGMSVGWIRQPPGKAL | 37 |
| | chain | EWLADIWWDDKKDYNPSLKSRLTISKDTSKNQVVLKVTNMDPADTA | |
| | | TYYCARSMITNWYFDVWGAGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVLPPSRDELTKNQVSLLCLVKGF | |
| | | YPSDIAVEWESNGQPENNYLTWPPVLDSDGSFFLYSKLTVDKSRWQQ | |
| | | GNVFSCSVMHEALHNHYTQKSLSLSPG | |
| CL_#11677 | Full heavy | GEVQLVESGGGLVQPGGSLRLSCAASGFSFSDFAMSWVRQAPGKGLE | 38 |
| | chain | WVATIGRVAFHTYYPDSMKGRFTISRDNSKNTLYLQMNSLRAEDTA | |
| | | VYYCARHRGFDVGHFDFWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVYPPSRDELTKNQVSLTCLVKGF | |
| | | YPSDIAVEWESNGQPENNYKTTPPVLDSDGSFALVSKLTVDKSRWQQ | |
| | | GNVFSCSVMHEALHNHYTQKSLSLSPG | |
| CL_#11678 | Full heavy | GEVQLVESGGGLVQPGGSLRLSCAASGFSFSDFAMSWVRQAPGKGLE | 39 |
| | chain | WVATIGRVAFHTYYPDSMKGRFTISRDNSKNTLYLQMNSLRAEDTA | |
| | | VYYCARHRGFDVGHFDFWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVLPPSRDELTKNQVSLLCLVKGF | |
| | | YPSDIAVEWESNGQPENNYLTWPPVLDSDGSFFLYSKLTVDKSRWQQ | |
| | | GNVFSCSVMHEALHNHYTQKSLSLSPG | |
| CL_#11695 | Full light | GDIQMTQSPSSLSASVGDRVTITCRSSETLVHSSGNTYLEWYQQKPGK | 40 |
| | chain | APKLLIYRVSNRFSGVPSRFSGSGSGTDFTLTISSLQPEDFATYYCFQGS | |
| | | FNPLTFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| CL_#11698 | Full heavy | GQVQLVQSGAEVVKPGASVKMSCKASGYTFTGYNIHWVKQAPGQG | 41 |
| | chain | LEWIGAIYPGNGDTSYKQKFRGRATLTADTSTSTVYMELSSLRSEDSA | |
| | | VYYCARGETARATFAYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVYPPSRDELTKNQVSLTCLVKGF | |
| | | YPSDIAVEWESNGQPENNYKTTPPVLDSDGSFALVSKLTVDKSRWQQ | |
| | | GNVFSCSVMHEALHNHYTQKSLSLSPG | |
| CL_#11699 | Full heavy | GQVQLVQSGAEVVKPGASVKMSCKASGYTFTGYNIHWVKQAPGQG | 42 |
| | chain | LEWIGAIYPGNGDTSYKQKFRGRATLTADTSTSTVYMELSSLRSEDSA | |
| | | VYYCARGETARATFAYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYVLPPSRDELTKNQVSLLCLVKGF | |
| | | YPSDIAVEWESNGQPENNYLTWPPVLDSDGSFFLYSKLTVDKSRWQQ | |
| | | GNVFSCSVMHEALHNHYTQKSLSLSPG | |
| CL_#11700 | Full light | GDIQMTQSPSSLSASVGDRVTITCSASQDIGNFLNWYQQKPGKTVKV | 43 |
| | chain | LIYYTSSLYSGVPSRFSGSGSGTDYTLTISSLQPEDFATYYCQQYSKLP | |
| | | LTFGQGTKLELKRTVAAPSVFIFPPSDEQLKSGTASVVCLLNNFYPRE | |
| | | AKVQWKVDNALQSGNSQESVTEQDSKDSTYSLSSTLTLSKADYEKH | |
| | | KVYACEVTHQGLSSPVTKSFNRGEC | |
| CL_#16603 | Full heavy | GQVTLRESGPALVKPTQTLTLTCTFSGFSLSTSGMSVGWIRQPPGKAL | 44 |
| | chain | EWLADIWWDDKKDYNPSLKSRLTISKDTSKNQVVLKVTNMDPADTA | |
| | | TYYCARSMITNWYFDVWGAGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#16635 | Full light | GDIQMTQSPSTLSASVGDRVTITCKSQLSVGYMHWYQQKPGKAPKLL | 45 |
| | chain | IYDTSKLASGVPSRFSGSGSGTEFTLTISSLQPDDFATYYCFQGSGYPFT | |
| | | FGGGTKLEIKRTVAAPSVFIFPPSDEQLKSGTASVVCLLNNFYPREAK | |
| | | VQWKVDNALQSGNSQESVTEQDSKDSTYSLSSTLTLSKADYEKHKV | |
| | | YACEVTHQGLSSPVTKSFNRGEC | |
| CL_#21503 | Full heavy | QVQLVQSGAEVKKPGASVKVSCKASGFTFTSYNVHWVRQAPGQGLE | 46 |
| | chain | WMGAISPGNGDLSYAQKFQGRVTMTRDTSTSTVYMELSSLRSEDTA | |
| | | VYYCARGRTGMGAVDYWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#21504 | Full heavy | QVQLVQSGAEVKKPGASVKVSCKASGFTFTSYNVHWVRQAPGQGLE | 47 |
| | chain | WIAAISPGNGDLSYAQKFQGRVTMTVDTSTSTVYMELSSLRSEDTAV | |
| | | YYCARGRTGMGAVDYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#21505 | Full heavy | QVQLVQSGAEVKKPGASVKMSCKASGFTFTSYNVHWVRQAPGQGL | 48 |
| | chain | EWIAAISPGNGDLSYAQKFQGRATLTVDTSTSTAYMELSSLRSEDTAV | |
| | | YYCARGRTGMGAVDYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#21506 | Full heavy | QVQLVQSGAEVKKPGASVKMSCKASGFTFTSYNVHWVRQAPGQGL | 49 |
| | chain | EWIAAISPGNGDLSYAQKFQGRATLTVDRSTSTAYMELSSLRSEDTAV | |
| | | YYCARGRTGMGAVDYWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCPAP | |
| | | ELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |
| CL_#21507 | Full light | DIQMTQSPSSLSASVGDRVTITCRASQDVYSYLNWYQQKPGKAPKLLI | 50 |
| | chain | YYTSRLQSGVPSRFSGSGSGTDFTLTISSLQPEDFATYYCQQYNIFPWT | |
| | | FGQGTKLEIKRTVAAPSVFIFPPSDEQLKSGTASVVCLLNNFYPREAK | |
| | | VQWKVDNALQSGNSQESVTEQDSKDSTYSLSSTLTLSKADYEKHKV | |
| | | YACEVTHQGLSSPVTKSFNRGEC | |
| CL_#21508 | Full light | DVQMTQSPSSLSASVGDRVTITCRASQDVYSYLNWYQQKPGKAVKL | 51 |
| | chain | LIYYTSRLQSGVPSRFSGSGSGTDYTLTISSLQPEDFATYYCQQYNIFP | |
| | | WTFGQGTKLEIKRTVAAPSVFIFPPSDEQLKSGTASVVCLLNNFYPRE | |
| | | AKVQWKVDNALQSGNSQESVTEQDSKDSTYSLSSTLTLSKADYEKH | |
| | | KVYACEVTHQGLSSPVTKSFNRGEC | |
| CL_#21509 | Full light | DVQMTQSPSSLSASVGDRVTITCRASQDVYSYLNWYQQKPGKAVKL | 52 |
| | chain | LIYYTSRLQSGVPSRFSGSGSGTDYTLTISSLQPEDIATYYCQQYNIFP | |
| | | WTFGQGTKLEIKRTVAAPSVFIFPPSDEQLKSGTASVVCLLNNFYPRE | |
| | | AKVQWKVDNALQSGNSQESVTEQDSKDSTYSLSSTLTLSKADYEKH | |
| | | KVYACEVTHQGLSSPVTKSFNRGEC | |
| CL_#29371 | Full heavy | QVQLVQSGAEVKKPGASVKVSCKASGFTFTSYNVHWVRQAPGQGLE | 61 |
| | chain | WMGAISPGNGDLSYAQKFQGRVTMTRDTSTSTVYMELSSLRSEDTA | |
| | v38591 | VYYCARGRTGMGAVDYWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPGK | |
| CL_#21508 | Full light | DVQMTQSPSSLSASVGDRVTITCRASQDVYSYLNWYQQKPGKAVKL | 62 |
| | chain | LIYYTSRLQSGVPSRFSGSGSGTDYTLTISSLQPEDFATYYCQQYNIFP | |
| | v29456 | WTFGQGTKLEIKRTVAAPSVFIFPPSDEQLKSGTASVVCLLNNFYPRE | |
| | and | AKVQWKVDNALQSGNSQESVTEQDSKDSTYSLSSTLTLSKADYEKH | |
| | v38591 | KVYACEVTHQGLSSPVTKSFNRGEC | |
| CL_#21503 | Full heavy | QVQLVQSGAEVKKPGASVKVSCKASGFTFTSYNVHWVRQAPGQGLE | 63 |
| | chain | WMGAISPGNGDLSYAQKFQGRVTMTRDTSTSTVYMELSSLRSEDTA | |
| | v29456 | VYYCARGRTGMGAVDYWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYV | |
| | | DGVEVHNAKTKPREEQYNSTYRVVSVLTVLHQDWLNGKEYKCKVS | |
| | | NKALPAPIEKTISKAKGQPREPQVYTLPPSRDELTKNQVSLTCLVKGF | |
| | | YPSDIAVEWESNGQPENNYKTTPPVLDSDGSFFLYSKLTVDKSRWQQ | |
| | | GNVFSCSVMHEALHNHYTQKSLSLSPG | |

TABLE C
Selected Clone DNA Sequences

| Clone | | | SEQ |
|---|---|---|---|
| Number | Region | Sequence | ID NO |
| CL_#21503 | VH | CAGGTGCAGCTGGTGCAGTCTGGAGCCGAGGTGAAGAAGCCAGGGG | 53 |
| | | CCAGCGTGAAGGTGTCCTGCAAGGCCTCTGGCTTCACCTTTACAAGC | |
| | | TACAACGTGCACTGGGTGAGGCAGGCCCCCGGACAGGGACTGGAGT | |
| | | GGATGGGAGCCATCAGCCCTGGCAATGGCGACCTGTCCTACGCCCA | |
| | | GAAGTTTCAGGGCAGGGTGACCATGACACGCGATACCTCCACATCT | |
| | | ACCGTGTATATGGAGCTGAGCTCCCTGAGATCCGAGGACACAGCCG | |
| | | TGTACTATTGTGCCCGGGGCAGAACCGGAATGGGAGCCGTGGATTA | |
| | | TTGGGGCCAGGGCACACTGGTGACCGTGTCTAGC | |
| CL_#21504 | VH | CAGGTGCAGCTGGTGCAGTCTGGAGCCGAGGTGAAGAAGCCAGGGG | 54 |
| | | CCAGCGTGAAGGTGTCCTGCAAGGCCTCTGGCTTCACCTTTACAAGC | |
| | | TACAACGTGCACTGGGTGCGGCAGGCCCCCGGACAGGGACTGGAGT | |
| | | GGATCGCAGCAATCAGCCCTGGCAATGGCGACCTGTCCTACGCCCA | |
| | | GAAGTTTCAGGGCCGGGTGACCATGACAGTGGATACCTCCACATCTA | |
| | | CCGTGTATATGGAGCTGAGCTCCCTGAGGTCCGAGGACACAGCCGT | |
| | | GTACTATTGTGCCCGGGGCAGAACCGGAATGGGAGCCGTGGATTAT | |
| | | TGGGGCCAGGGCACACTGGTGACCGTGTCTAGC | |
| CL_#21505 | VH | CAGGTGCAGCTGGTGCAGTCTGGAGCCGAGGTGAAGAAGCCAGGGG | 55 |
| | | CCAGCGTGAAGATGTCCTGCAAGGCCTCTGGCTTCACCTTTACAAGC | |
| | | TACAACGTGCACTGGGTGCGGCAGGCCCCCGGACAGGGACTGGAGT | |
| | | GGATCGCAGCAATCAGCCCTGGCAATGGCGACCTGTCCTACGCCCA | |
| | | GAAGTTTCAGGGCCGGGCCACCCTGACAGTGGATACCTCCACATCTA | |
| | | CCGCCTATATGGAGCTGAGCTCCCTGAGGTCCGAGGACACAGCCGT | |
| | | GTACTATTGTGCCCGGGGCAGAACCGGAATGGGAGCCGTGGATTAT | |
| | | TGGGGCCAGGGCACACTGGTGACCGTGTCTAGC | |
| CL_#21506 | VH | CAGGTGCAGCTGGTGCAGTCTGGAGCCGAGGTGAAGAAGCCAGGGG | 56 |
| | | CCAGCGTGAAGATGTCCTGCAAGGCCTCTGGCTTCACCTTTACAAGC | |
| | | TACAACGTGCACTGGGTGAGGCAGGCCCCCGGACAGGGACTGGAGT | |
| | | GGATCGCAGCAATCAGCCCTGGCAATGGCGACCTGTCCTACGCCCA | |
| | | GAAGTTTCAGGGCAGGGCCACCCTGACAGTGGATCGCTCCACCTCTA | |
| | | CAGCCTATATGGAGCTGAGCTCCCTGAGATCCGAGGACACCGCCGT | |
| | | GTACTATTGTGCCCGGGGCAGAACAGGAATGGGAGCCGTGGATTAT | |
| | | TGGGGCCAGGGCACCCTGGTGACAGTGTCTAGC | |
| CL_#21507 | VL | GACATCCAGATGACACAGTCTCCAAGCTCCCTGTCCGCCTCTGTGGG | 57 |
| | | CGACAGGGTGACCATCACATGCAGAGCCAGCCAGGACGTGTACTCC | |
| | | TATCTGAACTGGTACCAGCAGAAGCCCGGCAAGGCCCCTAAGCTGC | |
| | | TGATCTACTATACCAGCAGGCTGCAGTCCGGAGTGCCTTCTCGCTTC | |
| | | AGCGGCTCCGGCTCTGGAACCGACTTTACCCTGACAATCTCTAGCCT | |
| | | GCAGCCTGAGGATTTTGCCACATACTATTGTCAGCAGTATAATATCT | |
| | | TCCCATGGACCTTTGGCCAGGGCACAAAGCTGGAGATCAAG | |
| CL_#21508 | VL | GACGTGCAGATGACACAGTCTCCTAGCTCCCTGTCCGCCTCTGTGGG | 58 |
| | | CGACAGGGTGACCATCACATGCAGAGCCAGCCAGGACGTGTACTCC | |
| | | TATCTGAACTGGTACCAGCAGAAGCCTGGCAAGGCCGTGAAGCTGC | |
| | | TGATCTACTATACCAGCAGGCTGCAGTCCGGAGTGCCATCTCGCTTC | |
| | | AGCGGCTCCGGCTCTGGAACCGACTACACCCTGACAATCTCTAGCCT | |
| | | GCAGCCAGAGGATTTTGCCACATACTATTGTCAGCAGTATAATATCT | |
| | | TCCCCTGGACCTTTGGCCAGGGCACAAAGCTGGAGATCAAG | |
| CL_#21509 | VL | GACGTGCAGATGACACAGTCTCCTAGCTCCCTGTCCGCCTCTGTGGG | 59 |
| | | CGACAGGGTGACCATCACATGCAGAGCCAGCCAGGACGTGTACTCC | |
| | | TATCTGAACTGGTACCAGCAGAAGCCTGGCAAGGCCGTGAAGCTGC | |
| | | TGATCTACTATACCAGCAGGCTGCAGTCCGGCGTGCCATCTCGCTTT | |
| | | AGCGGCTCCGGCTCTGGAACCGACTACACCCTGACAATCTCTAGCCT | |
| | | GCAGCCAGAGGATATCGCCACATACTATTGTCAGCAGTATAATATCT | |
| | | TCCCCTGGACCTTTGGCCAGGGCACAAAGCTGGAGATCAAG | |
| CL_#29371 | Full | CAGGTGCAGCTGGTGCAGTCTGGAGCCGAGGTGAAGAAGCCAGGGG | 69 |
| | length | CCAGCGTGAAGGTGTCCTGCAAGGCCTCTGGCTTCACCTTTACAAGC | |
| | heavy | TACAACGTGCACTGGGTGAGGCAGGCCCCCGGACAGGGACTGGAGT | |
| | chain | GGATGGGAGCCATCAGCCCTGGCAATGGCGACCTGTCCTACGCCCA | |
| | v38591 | GAAGTTTCAGGGCAGGGTGACCATGACACGCGATACCTCCACATCT | |
| | | ACCGTGTATATGGAGCTGAGCTCCCTGAGATCCGAGGACACAGCCG | |
| | | TGTACTATTGTGCCCGGGGCAGAACCGGAATGGGAGCCGTGGATTA | |
| | | TTGGGGCCAGGGCACACTGGTGACCGTGTCTAGCGCTAGCACTAAG | |
| | | GGGCCTTCCGTGTTTCCACTGGCTCCCTCTAGTAAATCCACCTCTGGA | |
| | | GGCACAGCTGCACTGGGATGTCTGGTGAAGGATTACTTCCCTGAACC | |
| | | AGTCACAGTGAGTTGGAACTCAGGGGCTCTGACAAGTGGAGTCCAT | |
| | | ACTTTTCCCGCAGTGCTGCAGTCAAGCGGACTGTACTCCCTGTCCTCT | |
| | | GTGGTCACCGTGCCTAGTTCAAGCCTGGGCACCCAGACATATATCTG | |
| | | CAACGTGAATCACAAGCCATCAAATACAAAAGTCGACAAGAAAGTG | |
| | | GAGCCCAAGAGCTGTGATAAAACTCATACCTGCCCACCTTGTCCGGC | |
| | | GCCAGAACTGCTGGGAGGACCAAGCGTGTTCCTGTTTCCACCCAAGC | |
| | | CTAAAGACACCCTGATGATTTCCCGGACTCCTGAGGTCACCTGCGTG | |
| | | GTCGTGGACGTGTCTCACGAGGACCCCGAAGTCAAGTTCAACTGGTA | |
| | | CGTGGATGGCGTCGAAGTGCATAATGCCAAGACCAAACCCCGGGAG | |
| | | GAACAGTACAACTCTACCTATAGAGTCGTGAGTGTCCTGACAGTGCT | |
| | | GCACCAGGACTGGCTGAATGGGAAGGAGTATAAGTGTAAAGTGAGC | |
| | | AACAAAGCCCTGCCCGCCCCAATCGAAAAAACAATCTCTAAAGCAA | |
| | | AAGGACAGCCTCGCGAACCACAGGTGTACACTCTGCCTCCATCTCGG | |
| | | GACGAGCTGACTAAGAACCAGGTCAGTCTGACCTGTCTGGTGAAAG | |
| | | GATTCTATCCCAGCGATATCGCTGTGGAGTGGGAATCCAATGGCCAG | |
| | | CCTGAGAACAATTACAAGACCACACCCCCTGTGCTGGACTCTGATGG | |
| | | CAGTTTCTTTCTGTATAGTAAGCTGACCGTCGATAAATCACGATGGC | |
| | | AGCAGGGGAACGTGTTCAGCTGTTCAGTGATGCACGAAGCCCTGCA | |
| | | CAACCATTACACCCAGAAGAGCCTGAGCCTGTCTCCCGGCAAA | |

What is claimed is:

1. An antibody-drug conjugate having the structure: 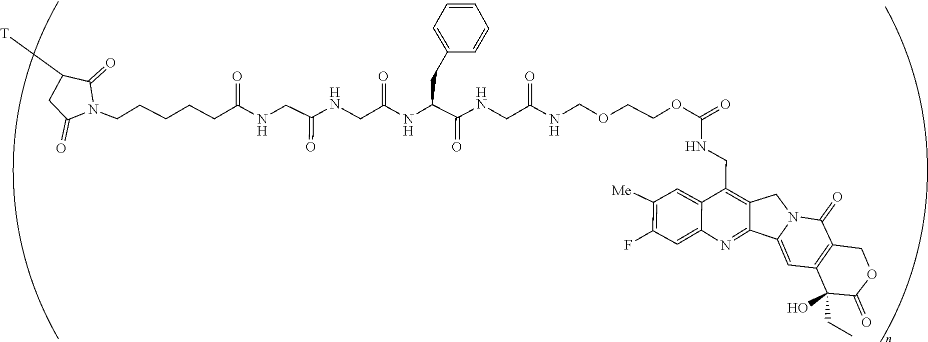
wherein n is between 2 and 6; and
T is an anti-NaPi2b (sodium-dependent phosphate transport protein 2B) antibody construct comprising an antigen-binding domain that binds to human NaPi2b, wherein the anti-NaPi2b antibody construct comprises two heavy chains comprising the sequence as set forth in SEQ ID NO:68, and two light chains comprising the sequence as set forth in SEQ ID NO:67, or wherein the anti-NaPi2b antibody construct comprises two heavy chains comprising the sequence as set forth in SEQ ID NO:66, and two light chains comprising the sequence as set forth in SEQ ID NO:67.

2. The antibody-drug conjugate according to claim 1, wherein n is between 3.5 and 4.5.

3. The antibody-drug conjugate according to claim 1, wherein n is about 4.

4. A pharmaceutical composition comprising an antibody-drug conjugate according to any one of claims 1 to 3, and a pharmaceutically acceptable carrier or diluent.

5. A method of inhibiting the proliferation of cancer cells comprising contacting the cells with an effective amount of the antibody-drug conjugate according to any one of claims 1 to 3.

6. A method of killing cancer cells comprising contacting the cells with an effective amount of the antibody-drug conjugate according to any one of claims 1 to 3.

7. A method of treating cancer in a subject in need thereof comprising administering to the subject an effective amount of the antibody-drug conjugate according to any one of claims 1 to 3.

8. Use of an effective amount of the antibody-drug conjugate according to any one of claims 1 to 3 for the treatment of cancer in a subject in need thereof.

9. An antibody-drug conjugate according to any one of claims 1 to 3 for use in therapy.

10. An antibody-drug conjugate according to any one of claims 1 to 3 for use in the treatment of cancer.

11. Use of an antibody-drug conjugate according to any one of claims 1 to 3 in the manufacture of a medicament for the treatment of cancer.

12. A kit comprising the antibody-drug conjugate according to any one of claims 1 to 3, and a label and/or package insert containing instructions for use.


SEQUENCE LISTING (71 sequences)

SEQ ID NO: 1 - protein, 690 aa, Homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL

SEQ ID NO: 2 - protein, 690 aa, Macaca fascicularis
MAPWPELGDAQPNPSKYLEGATSQQPITPDKSKETNKTDNTEVPVTKFELLPTYSTATLIEEPTEVDDPWNLPTLQDSGIKWSERDAKGKILCVFQGIGRLILLLGFLYFFVCSLDVLSSAFQLVGGKMAGQFFSNSSIMSNPLLGLVIGVLVTVFVQSSSTSTSIVVSMVASSLLTVRAAIPIIMGANIGTSITNTIVALMQVGDRSEFRRAFAGATVHDFFNWLSVLVLLPVEVATHYLEVVTQLIVESFHFKNGEDAPDLLKVITKPFTKLIVQLDKKVISQIAMNDETAKNKSLVKIWCKTFTNMTQMNVTVPSMANCTSHSLCWTDGIQIWTMKNVTYKENIAKCQHIFVNFHLPDLAVGIILLIISLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNTLRSSLQIALCHFFFNISGILLWYPIPFTRLPIRMAKGLGNISAKYRWFAVFYLIIFFFLIPLTVFGLSLAGWPVLVGVGVPVVFIIILVLCLRLLQSRCPRVLPKKLQNWNFLPLCMHSLKPWDAVISKFTGPFQMPCCCCCRVCCRVCCLLCGCPKCCRCSKCCQDLEEGQEAQGVPVKAPETFDNITISREAQGEVRAPDSKTECTAL

SEQ ID NO: 3 - protein, 697 aa, Mus musculus
MAPWPELENAQPNPGKFIEGASGPQSSIPAKDKEASKTNDNGTPVAKTELLPSYSALVLIEEHPEGTDPWDLPELQDTGIKWSERDTKGKTLCIFQGVGKFILLLGFLYLFVCSLDVLSSAFQLVGGKVAGQFFSNNSIMSNPVAGLVIGVLVTVMVQSSSTSSSIIVSMVASSLLTVRAAIPIIMGANIGTSITNTIVALMQAGDRNEFRRAFAGATVHDFFNWLSVFVLLPLEAATHYLEILTNLVLETFKFQNGEDAPDILKVITDPFTKLIIQLDKKVIQQIAMGDSAAQNKSLIKIWCKSITNVTEMNVTVPSTDNCTSPSYCWTDGIQTWTIQNVTQKENIAKCQHIFVNFSLPDLAVGIILLTVSLVVLCGCLIMIVKLLGSVLRGQVATVIKKTLNTDFPFPFAWLTGYLAILVGAGMTFIVQSSSVFTSAMTPLIGIGVISIERAYPLTLGSNIGTTTTAILAALASPGNTLRSSLQIALCHFFFNISGILLWYPIPFTRLPIRLAKGLGNISAKYRWFAVFYLIFFFFVTPLTVFGLSLAGWPVLVGVGVPIILLLLLVLCLRMLQFRCPRILPLKLRDWNFLPLWMHSLKPWDNVISLATTCFQRRCCCCCRVCCRVCCMVCGCKCCRCSKCCRDQGEEEEEKEQDIPVKASGAFDNAAMSKECQDEGKGQVEVLSMKALSNTTVF

SEQ ID NO: 4 - protein, 8 aa, synthetic construct
  source 1..8: note = IMGT VH CDR1
GFTFTSYN

SEQ ID NO: 5 - protein, 8 aa, synthetic construct
  source 1..8: note = IMGT VH CDR2
ISPGNGDL

SEQ ID NO: 6 - protein, 12 aa, synthetic construct
  source 1..12: note = IMGT VH CDR3
ARGRTGMGAVDY

SEQ ID NO: 7 - protein, 5 aa, synthetic construct
  source 1..5: note = Kabat VH CDR1
SYNVH

SEQ ID NO: 8 - protein, 17 aa, synthetic construct
  source 1..17: note = Kabat VH CDR2
AISPGNGDLSYNQRFKD

SEQ ID NO: 9 - protein, 10 aa, synthetic construct
  source 1..10: note = Kabat, Chothia, AbM VH CDR3
GRTGMGAVDY

SEQ ID NO: 10 - protein, 7 aa, synthetic construct
  source 1..7: note = Chothia VH CDR1
GFTFTSY

SEQ ID NO: 11 - protein, 6 aa, synthetic construct
  source 1..6: note = Chothia VH CDR2
SPGNGD

SEQ ID NO: 12 - protein, 10 aa, synthetic construct
  source 1..10: note = AbM VH CDR1
GFTFTSYNVH

SEQ ID NO: 13 - protein, 10 aa, synthetic construct
  source 1..10: note = AbM VH CDR2
AISPGNGDLS

SEQ ID NO: 14 - protein, 6 aa, synthetic construct
  source 1..6: note = Contact VH CDR1
TSYNVH

SEQ ID NO: 15 - protein, 13 aa, synthetic construct
  source 1..13: note = Contact VH CDR2
WIAAISPGNGDLS

SEQ ID NO: 16 - protein, 11 aa, synthetic construct
  source 1..11: note = Contact VH CDR 3
ARGRTGMGAVD

SEQ ID NO: 17 - protein, 6 aa, synthetic construct
  source 1..6: note = IMGT VL CDR1
QDVYSY

SEQ ID NO: 18 - protein, 9 aa, synthetic construct
  source 1..9: note = IMGT, Kabat, Chothia, AbM VL CDR3
QQYNIFPWT

SEQ ID NO: 19 - protein, 11 aa, synthetic construct
  source 1..11: note = Kabat, Chothia, AbM VL CDR1
RASQDVYSYLN

SEQ ID NO: 20 - protein, 7 aa, synthetic construct
  source 1..7: note = Kabat, Chothia, AbM VL CDR2
YTSRLQS

SEQ ID NO: 21 - protein, 7 aa, synthetic construct
  source 1..7: note = Contact VL CDR1
YSYLNWY

SEQ ID NO: 22 - protein, 10 aa, synthetic construct
  source 1..10: note = Contact VL CDR2
LLIYYTSRLQ

SEQ ID NO: 23 - protein, 8 aa, synthetic construct
  source 1..8: note = Contact VL CDR3
QQYNIFPW

SEQ ID NO: 24 - protein, 119 aa, synthetic construct
  source 1..119: note = v29452, v29460 H1
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSS

SEQ ID NO: 25 - protein, 119 aa, synthetic construct
  source 1..119: note = v29451, v29455, v29459 H2
QVQLVQSGAEVKKPGASVKVSCKASGFTFTSYNVHWVRQAPGQGLEWIAAISPGNGDLSYAQKFQGRVTMTVDTSTSTVYMELSSLRSEDTAVYYCARGRTGMGAVDYWGQGTLVTVSS

SEQ ID NO: 26 - protein, 119 aa, synthetic construct
  source 1..119: note = v29450, v29454, v29458 H3
QVQLVQSGAEVKKPGASVKMSCKASGFTFTSYNVHWVRQAPGQGLEWIAAISPGNGDLSYAQKFQGRATLTVDTSTSTAYMELSSLRSEDTAVYYCARGRTGMGAVDYWGQGTLVTVSS

SEQ ID NO: 27 - protein, 119 aa, synthetic construct
  source 1..119: note = v29449, v29453, v29457 H4
QVQLVQSGAEVKKPGASVKMSCKASGFTFTSYNVHWVRQAPGQGLEWIAAISPGNGDLSYAQKFQGRATLTVDRSTSTAYMELSSLRSEDTAVYYCARGRTGMGAVDYWGQGTLVTVSS

SEQ ID NO: 28 - protein, 107 aa, synthetic construct
  source 1..107: note = v29457, v29458, v29459, v29460 L1
DIQMTQSPSSLSASVGDRVTITCRASQDVYSYLNWYQQKPGKAPKLLIYYTSRLQSGVPSRFSGSGSGTDFTLTISSLQPEDFATYYCQQYNIFPWTFGQGTKLEIK

SEQ ID NO: 29 - protein, 107 aa, synthetic construct
  source 1..107: note = v29453, v29454, v29455, v29456 L2
DVQMTQSPSSLSASVGDRVTITCRASQDVYSYLNWYQQKPGKAVKLLIYYTSRLQSGVPSRFSGSGSGTDYTLTISSLQPEDFATYYCQQYNIFPWTFGQGTKLEIK

SEQ ID NO: 30 - protein, 107 aa, synthetic construct
  source 1..107: note = v29449 L3
DVQMTQSPSSLSASVGDRVTITCRASQDVYSYLNWYQQKPGKAVKLLIYYTSRLQSGVPSRFSGSGSGTDYTLTISSLQPEDIATYYCQQYNIFPWTFGQGTKLEIK

SEQ ID NO: 31 - protein, 119 aa, synthetic construct
  source 1..119: note = v23855 VH
QAYLQQSGAELVRPGASVKMSCKASGFTFTSYNVHWVKQTPRQGLEWIAAISPGNGDLSYNQRFKDKATLTVDRSSSTAYMQLSGLTSEDSAVYFCARGRTGMGAVDYWGQGTSVTVSS

SEQ ID NO: 32 - protein, 107 aa, synthetic construct
  source 1..107: note = v23855 VL
DVQMTQTASSLSASLGDRVTISCRASQDVYSYLNWYQQKPHGTVKLLIYYTSRLQSGVPSRFTGSGSGTDYSLTISNLEPEDIATYYCQQYNIFPWTFGGGTKLEIK

SEQ ID NO: 33 - protein, 329 aa, synthetic construct
  source 1..329: note = IGHG1_01
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 107 aa, synthetic construct [sequence omitted]

SEQ ID NO: 35 - protein, 23 aa, synthetic construct
  source 1..23: note = signal peptide
MRPTWAWWLFLVLLLALWAPARG

SEQ ID NO: 36 - protein, 450 aa, synthetic construct
  source 1..450: note = Clone ID 11009 Full H1
GQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 37 - protein, 450 aa, synthetic construct
  source 1..450: note = Clone ID 11010 Full H2
GQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 38 - protein, 450 aa, synthetic construct
  source 1..450: note = Clone ID 11677 Full H1
GEVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 39 - protein, 450 aa, synthetic construct
  source 1..450: note = Clone ID 11678 Full H2
GEVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 40 - protein, 220 aa, synthetic construct
  source 1..220: note = Clone ID 11695 Full L1, L2
GDIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 449 aa, synthetic construct
  source 1..449: note = Clone ID 11698 Full H1
GQVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 42 - protein, 449 aa, synthetic construct
  source 1..449: note = Clone ID 11699 Full H2
GQVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 43 - protein, 215 aa, synthetic construct
  source 1..215: note = Clone ID 11700 Full L1, L2
GDIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 450 aa, synthetic construct
  source 1..450: note = Clone ID 16603 Full H1, H2
GQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 214 aa, synthetic construct
  source 1..214: note = Clone ID 16635 Full L1, L2
GDIQMTQSPSTLSASVGDRVTITCKSQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 448 aa, synthetic construct
  source 1..448: note = Clone ID 21503 Full H1, H2
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 448 aa, synthetic construct
  source 1..448: note = Clone ID 21504 Full H1, H2
QVQLVQSGAEVKKPGASVKVSCKASGFTFTSYNVHWVRQAPGQGLEWIAAISPGNGDLSYAQKFQGRVTMTVDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 448 aa, synthetic construct
  source 1..448: note = QVQLVQSGAEVKKPGASVKMSCKASGFTFTSYNVHWVRQAPGQGLEWIAAI SPGNGDLSYAQKFQGRATLTVDTSTSTAYMELSSLRSEDTAVYYCARGRTGMGAVDYW GQGTLVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKKVEPKSCDKT HTCPPCPAPELLGGPSVFLFPPKPKDTLMISRTPEVTCVVVDVSHEDPEVKFNWYVDG VEVHNAKTKPREEQYNSTYRVVSVLTVLHQDWLNGKEYKCKVSNKALPAPIEKTISKA KGQPREPQVYTLPPSRDELTKNQVSLTCLVKGFYPSDIAVEWESNGQPENNYKTTPPV LDSDGSFFLYSKLTVDKSRWQQGNVFSCSVMHEALHNHYTQKSLSLSPG
QVQLVQSGAEVKKPGASVKMSCKASGFTFTSYNVHWVRQAPGQGLEWIAAISPGNGDLSYAQKFQGRATLTVDTSTSTAYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 448 aa, synthetic construct
  source 1..448: note = Clone ID 21506 Full H1, H2
QVQLVQSGAEVKKPGASVKMSCKASGFTFTSYNVHWVRQAPGQGLEWIAAISPGNGDLSYAQKFQGRATLTVDRSTSTAYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 214 aa, synthetic construct
  source 1..214: note = Clone ID 21507 Full L1, L2
DIQMTQSPSSLSASVGDRVTITCRASQDVYSYLNWYQQKPGKAPKLLIYYTSRLQSGVPSRFSGSGSGTDFTLTISSLQPEDFATYYCQQYNIFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, synthetic construct
  source 1..214: note = Clone ID 21508 Full L1, L2
DVQMTQSPSSLSASVGDRVTITCRASQDVYSYLNWYQQKPGKAVKLLIYYTSRLQSGVPSRFSGSGSGTDYTLTISSLQPEDFATYYCQQYNIFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, synthetic construct
  source 1..214: note = Clone ID 21509 Full L1, L2
DVQMTQSPSSLSASVGDRVTITCRASQDVYSYLNWYQQKPGKAVKLLIYYTSRLQSGVPSRFSGSGSGTDYTLTISSLQPEDIATYYCQQYNIFPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 357 bp, synthetic construct
  source 1..357: mol_type = other DNA; note = Clone ID 21503 VH
caggtgcagctggtgcagtctggagccgaggtgaagaagccaggggccagcgtgaaggtgtcctgcaaggcctctggcttcacctttacaagctacaacgtgcactgggtgaggcaggcccccggacagggactggagtggatgggagccatcagccctggcaatggcgacctgtcctacgcccagaagtttcagggcagggtgaccatgacacgcgatacctccacatctaccgtgtatatggagctgagctccctgagatccgaggacacagccgtgtactattgtgcccggggcagaaccggaatgggagccgtggattattggggccagggcacactggtgaccgtgtctagc

SEQ ID NO: 54 - DNA, 357 bp, synthetic construct
  source 1..357: mol_type = other DNA; note = Clone ID 21504 VH
caggtgcagctggtgcagtctggagccgaggtgaagaagccaggggccagcgtgaaggtgtcctgcaaggcctctggcttcacctttacaagctacaacgtgcactgggtgcggcaggcccccggacagggactggagtggatcgcagcaatcagccctggcaatggcgacctgtcctacgcccagaagtttcagggccgggtgaccatgacagtggatacctccacatctaccgtgtatatggagctgagctccctgaggtccgaggacacagccgtgtactattgtgcccggggcagaaccggaatgggagccgtggattattggggccagggcacactggtgaccgtgtctagc

SEQ ID NO: 55 - DNA, 357 bp, synthetic construct
  source 1..357: mol_type = other DNA; note = Clone ID 21505 VH
caggtgcagctggtgcagtctggagccgaggtgaagaagccaggggccagcgtgaagatgtcctgcaaggcctctggcttcacctttacaagctacaacgtgcactgggtgcggcaggcccccggacagggactggagtggatcgcagcaatcagccctggcaatggcgacctgtcctacgcccagaagtttcagggccgggccaccctgacagtggatacctccacatctaccgcctatatggagctgagctccctgaggtccgaggacacagccgtgtactattgtgcccggggcagaaccggaatgggagccgtggattattggggccagggcacactggtgaccgtgtctagc

SEQ ID NO: 56 - DNA, 357 bp, synthetic construct
  source 1..357: mol_type = other DNA; note = Clone ID 21506 VH
caggtgcagctggtgcagtctggagccgaggtgaagaagccaggggccagcgtgaagatgtcctgcaaggcctctggcttcacctttacaagctacaacgtgcactgggtgaggcaggcccccggacagggactggagtggatcgcagcaatcagccctggcaatggcgacctgtcctacgcccagaagtttcagggcagggccaccctgacagtggatcgctccacctctacagcctatatggagctgagctccctgagatccgaggacaccgccgtgtactattgtgcccggggcagaacaggaatgggagccgtggattattggggccagggcaccctggtgacagtgtctagc

SEQ ID NO: 57 - DNA, 321 bp, synthetic construct
  source 1..321: mol_type = other DNA; note = Clone ID 21507 VL
gacatccagatgacacagtctccaagctccctgtccgcctctgtgggcgacagggtgaccatcacatgcagagccagccaggacgtgtactcctatctgaactggtaccagcagaagcccggcaaggcccctaagctgctgatctactataccagcaggctgcagtccggagtgccttctcgcttcagcggctccggctctggaaccgactttaccctgacaatctctagcctgcagcctgaggattttgccacatactattgtcagcagtataatatcttcccatggacctttggccagggcacaaagctggagatcaag

SEQ ID NO: 58 - DNA, 321 bp, synthetic construct
  source 1..321: mol_type = other DNA; note = Clone ID 21508 VL
gacgtgcagatgacacagtctcctagctccctgtccgcctctgtgggcgacagggtgaccatcacatgcagagccagccaggacgtgtactcctatctgaactggtaccagcagaagcctggcaaggccgtgaagctgctgatctactataccagcaggctgcagtccggagtgccatctcgcttcagcggctccggctctggaaccgactacaccctgacaatctctagcctgcagccagaggattttgccacatactattgtcagcagtataatatcttcccctggacctttggccagggcacaaagctggagatcaag

SEQ ID NO: 59 - DNA, 321 bp, synthetic construct
  source 1..321: mol_type = other DNA; note = Clone ID 21509 VL
gacgtgcagatgacacagtctcctagctccctgtccgcctctgtgggcgacagggtgaccatcacatgcagagccagccaggacgtgtactcctatctgaactggtaccagcagaagcctggcaaggccgtgaagctgctgatctactataccagcaggctgcagtccggcgtgccatctcgctttagcggctccggctctggaaccgactacaccctgacaatctctagcctgcagccagaggatatcgccacatactattgtcagcagtataatatcttcccctggacctttggccagggcacaaagctggagatcaag

SEQ ID NO: 60 - protein, 217 aa, synthetic construct
  source 1..217: note = Human IgG1 Fc Sequence1 and CH3 Domain Amino Acid Modifications Promoting Heterodimer Formation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 449 aa, synthetic construct
  source 1..449: note = Clone ID 29371 Full H1, H2 v38591
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, synthetic construct; identical to SEQ ID NO: 51
  source 1..214: note = Clone ID 21503 Full H1, H2 v29456

SEQ ID NO: 63 - protein, 448 aa, synthetic construct; identical to SEQ ID NO: 46
  source 1..448: note = Clone ID 21503 Full H1, H2 v29456

SEQ ID NO: 64 - protein, 330 aa, synthetic construct
  source 1..330: note = IGHG1_01 with L234A L235A D265S
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 107 aa, synthetic construct [sequence omitted]

SEQ ID NO: 66 - protein, 449 aa, synthetic construct
  source 1..449: note = v40502 HC
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 214 aa, synthetic construct; identical to SEQ ID NO: 51
  source 1..214: note = v40502 LC

SEQ ID NO: 68 - protein, 448 aa, synthetic construct
  source 1..448: note = HC (without C-terminal lysine)
QVQLVQSGAEVKKPGASVKVSCKASGFTFTSYNVHWVRQAPGQGLEWMGAISPGNGDLSYAQKFQGRVTMTRDTSTSTVYMELSSLRSEDTAVYYCARGRTGMGAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - DNA, 1347 bp, synthetic construct
  source 1..1347: mol_type = other DNA; note = Clone ID 29371 Full H1, H2 v38591
caggtgcagctggtgcagtctggagccgaggtgaagaagccaggggccagcgtgaaggtgtcctgcaaggcctctggcttcacctttacaagctacaacgtgcactgggtgaggcaggcccccggacagggactggagtggatgggagccatcagccctggcaatggcgacctgtcctacgcccagaagtttcagggcagggtgaccatgacacgcgatacctccacatctaccgtgtatatggagctgagctccctgagatccgaggacacagccgtgtactattgtgcccggggcagaaccggaatgggagccgtggattattggggccagggcacactggtgaccgtgtctagcgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtgtacactctgcctccatctcgggacgagctgactaagaaccaggtcagtctgacctgtctggtgaaaggattctatcccagcgatatcgctgtggagtgggaatccaatggccagcctgagaacaattacaagaccacaccccctgtgctggactctgatggcagtttctttctgtatagtaagctgaccgtcgataaatcacgatggcagcaggggaacgtgttcagctgttcagtgatgcacgaagccctgcacaaccattacacccagaagagcctgagcctgtctcccggcaaa

SEQ ID NO: 70 - protein, 119 aa, synthetic construct; identical to SEQ ID NO: 24
  source 1..119: note = IGHV1-46_03

SEQ ID NO: 71 - protein, 107 aa, synthetic construct; identical to SEQ ID NO: 28
  source 1..107: note = IGKVID-39_01